(19) United States
(12) Patent Application Publication
(10) Pub. No.: US 2024/0409950 A1
(43) Pub. Date: Dec. 12, 2024

(54) POLYPLOID HYBRID BREEDING

(71) Applicant: Ohalo Genetics Inc., Aptos, CA (US)

(72) Inventors: Judson Arthur WARD, Watsonville, CA (US); Jason A. Peiffer, Hendersonville, NC (US); Lien Diana Bertier, Santa Cruz, CA (US); Morgan Edward MCCAW, Aptos, CA (US); Xingang Wang, Aromas, CA (US); Zachary Campbell Bower, Aptos, CA (US); Benjamin Thomas Berube, Scotts Valley, CA (US)

(73) Assignee: Ohalo Genetics Inc., Aptos, CA (US)

(21) Appl. No.: 18/595,300

(22) Filed: Mar. 4, 2024

Related U.S. Application Data
(63) Continuation of application No. PCT/US2023/079146, filed on Nov. 8, 2023.
(60) Provisional application No. 63/497,670, filed on Apr. 21, 2023.
(60) Provisional application No. 63/461,174, filed on Apr. 21, 2023.
(60) Provisional application No. 63/423,768, filed on Nov. 8, 2022.
(60) Provisional application No. 63/423,765, filed on Nov. 8, 2022.

(51) Int. Cl. C12N 15/82; C12N 9/22; C12N 15/11

(52) U.S. Cl. CPC C12N 15/8262; C12N 9/22; C12N 15/11; C12N 15/8213; C12N 2310/20

(57) ABSTRACT

The present inventions relate to a breeding system for the production of polyploid seeds, plants, or plant parts where cycles of meiosis, syngamy, and selection are used for interpopulation improvement of progenitor lines, and sexual polyploidization occurs during hybrid production by inducing clonal gamete formation in the parents that are to be crossed. Reciprocal recurrent selection can be used to inform selection of candidate lines that are either advanced to a gene editing or genetic modification system or crossed and selected to induce clonal gamete formation by arresting meiotic recombination and chromosome reduction. Crosses of parent plants bearing clonal gametes are planned and executed based upon predicted heterotic performance at the polyploid level. The final product is a homogeneous population of hybrid polyploid seed, or derivative thereof, bearing both parents' complete nuclear genomes. In some instances, the method is used to generate plants that produce seedless fruit.

### CROSS-REFERENCE TO RELATED APPLICATIONS

This application is a continuation of International Application Number PCT/US2023/079146, filed Nov. 8, 2023, which claims the benefit of priority to U.S. Provisional Application No. 63/423,765, filed on Nov. 8, 2022, to U.S. Provisional Application No. 63/423,768, filed on Nov. 8, 2022, to U.S. Provisional Application No. 63/497,670, filed Apr. 21, 2023, and to U.S. Provisional Application No. 63/461,174, filed Apr. 21, 2023, each of which is incorporated by reference herein in its entirety.

### REFERENCE TO AN ELECTRONIC SEQUENCE LISTING

The content of the electronic sequence listing (197072000101seglist.xml; Size: 523,525 bytes; and Date of Creation: Nov. 13, 2023) is herein incorporated by reference in its entirety.

### FIELD

The inventions relate generally to the field of agricultural science, and specifically to crop improvement and systems of breeding novel hybrid polyploid plant cultivars. The inventions also relate to population improvement methods to desirably alter the genetic composition of diploid breeding populations for accelerated production of uniform hybrid polyploid seeds, plants, and plant parts suitable for cultivation. The inventions further relate to plant materials obtained by this process.

### BACKGROUND

The growing human population, a desire to reduce the environmental impact of agriculture, and consumer food preferences ensure a constant need for improved varieties of crops. Crop breeding has been used as a way to improve crop characteristics, yields, and robustness to environmental pressure for millennia. Despite growth in crop yields in the 20^{th} century, crop yields have begun to plateau in recent decades, signaling a need for improved breeding methods (e.g., Rizzo (2021). Climate and agronomy, not genetics, underpin recent maize yield gains in favorable environments. Proceedings of the National Academy of Sciences 119(4): e2113629119). In some staple and specialty crops, the most widely-grown cultivars are polyploid. For example, the most preferred and widely-grown varieties of potato are autotetraploids, and the Cavendish banana, which accounts for about half of global banana production, is triploid. This creates immense complexity in breeding such varieties due to the increased inefficiency of artificial selection in polyploid plant species, and, in some cases, the requirement for vegetative propagation of the varieties, which is less desirable compared to seed-based propagation. For example, because the Cavendish banana is a seedless triploid, it must be reproduced through vegetative propagation of identical clones. The resulting lack of genetic diversity in banana production makes this crop highly vulnerable to disease outbreaks and other environmental stresses. For such polyploid crops, there is an even greater need for innovative breeding techniques for the development of improved varieties.

Hybrid crops are widely grown and preferred because they tend to exhibit more robust growth, higher yields, and resilience to environmental stressors compared to their inbred or open-pollinated counterparts. This phenomenon is known as heterosis, or “hybrid vigor”, and reflects the tendency of a cross-bred plant to show superior quality due to extensive heterozygosity in the plant's genome. Hybrid seed is typically produced by a single cross of fully inbred parent plants with different sets of alleles, resulting in a biallelic hybrid plant with two sets of alleles (also known as haplotypes) contributing to heterosis. In a further extension of the mechanism of heterosis, polyploid crops can exhibit progressive heterosis; for example, the additional hybrid vigor in a multiallelic double-cross tetraploid hybrid plant that is not found in its biallelic single-cross tetraploid parents or in its more inbred grandparents. Progressive heterosis has been documented in a number of tetraploid species including alfalfa, potato, and tetraploid maize (Gallais. (1984). An analysis of heterosis vs. inbreeding effects with an autotetraploid cross-fertilized plant: Medicago sativa L. Genetics 106, 123-137; Groose et al. 1989. Progressive heterosis in autotetraploid alfalfa: studies using two types of inbreds. Crop Sci. 29, 1173-1177; Mok and Peloquin. 1975. Breeding value of 2n pollen (diplandroids) in tetraploid x diploid crosses in potatoes. Theor. Appl. Genet. 46, 307-314; Washburn et al. 2013. Polyploids as a “model system” for the study of heterosis. Plant Reprod 27:1-5; Washburn et al. 2019. Progressive heterosis in genetically defined tetraploid maize (J Genet Genomics. 46(8):389-396) has resulted in increased above ground biomass and several other agronomically desirable traits. However, due to the need to cross heterozygous single-cross parents to generate double-cross polyploid hybrids, it is not feasible to generate a uniform population of true-breeding seed while taking advantage of progressive heterosis with current breeding techniques.

Fruit development in most crops relies on signals from developing seeds to stimulate growth, but certain crops may develop fruits in the absence of a viable seed. This phenomenon is known as parthenocarpy and may take various forms. While the mechanisms of parthenocarpy vary from crop to crop, one commonality in seedless cultivars is pairing of a parthenocarpic system with either self-incompatibility and the prohibition of cross-pollination or complete or near complete sterility of pollen and/or egg cells. In parthenocarpic crops, low gametic viability nearly guarantees seedless fruit production and is commonly achieved by generating triploid plants resulting from crosses between homozygous diploid pollinators and homozygous tetraploid maternal plants. A chief mechanism responsible for the low gametic viability of triploid plants is nondisjunction. Among other reasons, this can occur because the three homologs of each chromosome join and cross-over to produce trivalents during the first meiotic division. The resulting chromosome segregation of each trivalent into two daughter nuclei is random. This causes it to be highly unlikely that a sufficiently large number of viable genetically balanced gamete cells can be produced at the end of meiosis.

Although they bear the coveted seedless fruit, triploid systems have several weaknesses. Triploid seed often exhibits problems with viability resulting from atypical contributions of the maternal and paternal genomes to that of the endosperm. In a normal diploid by diploid cross, the embryo is diploid with a triploid endosperm due to the double fertilization process in which a sperm cell of the pollen fuses with the two polar nuclei of the female gametophyte to form the endosperm (2:1 maternal:paternal contribution of genomes). By contrast, a cross between a diploid pollinator and a tetraploid mother would yield the desired triploid embryo with a pentaploid endosperm (4:1 maternal:paternal contribution of genomes). This ratio of maternal to paternal contributions of genomes to the endosperm results in abnormal seed development and, often, reduced viability. Another major weakness of triploid systems is that, in order to produce a uniform population of triploid seed, both the tetraploid and diploid parents must be highly homozygous, which limits the number of haplotypes in these individuals to two.

Prior research has established methods that allow plant geneticists to arrest meiosis in plants and replace it with a mitosis-like division in germline cells, resulting in formation of clonal gametes that contain the complete nonrecombinant genome of the parent. One such method, known as MiMe (Mitosis instead of Meiosis; d'Erfurth et al. 2009. Turning meiosis into mitosis. PLoS Biol 7, no. 6: e1000124.) is achieved through a triple knockout of three genes encoding gene products involved in meiosis, specifically, (1) a component of sister chromatid cohesion during the first division of meiosis, (2) a component of DNA double strand breakage during meiotic recombination, and (3) a component of progression through the second division of meiosis. This technology for MiMe is well-developed, but commercial development of MiMe has only begun in a select few applications (e.g., see US20190098858A1, US20120042408A1, and U.S. Ser. No. 10/883,112B2). These current applications of MiMe that are in development have only focused on generation of apomictic seed from diploid plants. However, the application of MiMe to develop novel and enhanced breeding techniques for plants at the polyploid level, including polyploid parthenocarpic plants, has not yet been realized.

### BRIEF SUMMARY

Provided herein are novel methods of breeding polyploid plants and genetically uniform plant populations that apply MiMe in innovative ways to accelerate breeding and produce plants or plant populations that are either very difficult to produce by traditional breeding or simply cannot be produced by traditional breeding.

In particular, certain methods leverage the production of clonal gametes to unlock the potential of progressive heterosis in breeding of polyploid crops. First, a diverse set of plant lines of a given ploidy is obtained, improved using traditional breeding methods, and organized into heterotic groups based on the predicted heterotic performance of their haplotypes when combined in plants of higher ploidy. Then, candidate lines together comprising three or more haplotypes are selected from the set of plant lines, and one or more candidate lines are induced to form clonal gametes by a method such as MiMe. The clonal gametes are then crossed (for example, with other gametes such as clonal gametes, haploid gametes derived from a fully inbred individual, or other types of unreduced gametes that would result in three or more haplotypes) to produce a homogenous population of multiallelic polyploid seed comprising the three or more haplotypes of the candidate lines. The polyploid seed is then grown, the plants are evaluated for the characteristics desired in the breeding program, and the heterotic performance of the haplotypes comprised by the plants is used to guide the breeding and selection of lines for further rounds of breeding. Exemplary embodiments of these methods are depicted in FIGS. 16-24C. For many species this method allows, for the first time, the production of genetically uniform seed of a crop comprising three or more haplotypes, thus allowing for practical application of progressive heterosis in polyploids.

Certain related methods provided herein leverage the production of clonal gametes to unlock the potential of progressive heterosis in breeding of seedless polyploid crops, including parthenocarpic crops. The method may be applied to the breeding and production of genetically-uniform populations of polyploid seeds yielding seedless plants, which provides advantages including, but not limited to, a means of manipulating source-sink carbon flux in root vegetable and tuber crops, a means of leveraging progressive heterosis in breeding improved cultivars of parthenocarpic fruit crops, and a means of producing uniform populations of parthenocarpic fruit crop seed. In such methods, candidate lines, which may together comprise two, three, four, or more haplotypes, are selected, and two or more candidate lines are induced to form clonal gametes by a method such as MiMe, to produce two parent MiMe plants each having MiMe alleles at two or more MiMe loci on each set of chromosomes that confer clonal gamete formation. The parent MiMe plants are selected such that a) the wild-type (non-MiMe) alleles of the first parent MiMe plants partially complement the MiMe alleles conferring clonal gamete formation of the second parent MiMe plant, and vice versa; and b) the parent MiMe plants have at least one MiMe locus in common with MiMe alleles at that locus on all sets of chromosomes. The clonal gametes are then crossed to produce a homogenous population of multiallelic polyploid seed comprising the two, three, or more haplotypes candidate lines, which, due to the genotypes of the parent MiMe plants, have only MiMe alleles at one or more MiMe loci on all sets of chromosomes, and at least one non-MiMe allele at all other MiMe loci. Thus, the population of polyploid seed has neither a normal meiosis phenotype nor a clonal gamete formation phenotype, and therefore produces inviable gametes and seedless plants. The polyploid seed is then grown, the plants are evaluated for the characteristics desired in the breeding program, and the heterotic performance of the haplotypes comprised by the plants is used to guide the breeding and selection of lines for further rounds of breeding. Exemplary embodiments of these methods are depicted in FIGS. 16-24C. For many species this method allows, for the first time, the production of genetically uniform seed of a crop comprising three or more haplotypes which, upon germination, produce plants bearing inviable gametes, thus allowing for practical application of progressive heterosis in polyploids, including parthenocarpic crops. Additionally, this method provides an alternative to vegetative propagation of seedless parthenocarpic fruit crops by allowing for the production of true-seed populations of improved parthenocarpic fruit crop hybrids, addressing a long-felt need for increased genetic diversity in such crops.

In one aspect, the present disclosure provides a population of polyploid seed comprising three or more haplotypes of the same or related species of plant, wherein at least 50% of the population of polyploid seed are genetically uniform, and wherein the population was obtained from a single plant or a set of plants such as, for example, a set of F1 hybrids. In some embodiments, the present disclosure provides a population of polyploid seed comprising a subpopulation of genetically uniform polyploid seed in an amount of at least 50% of the total number of seeds, the genetically uniform polyploid seed comprising three or more haplotypes of the same or related species of plant, wherein the population was obtained from a single plant or a set of plants such as, for example, of F1 hybrids. The polyploid seed (e.g., the subpopulation of genetically uniform seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid seed are genetically uniform. In certain embodiments, the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid seed comprises the subpopulation of genetically uniform polyploid seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid seed has a pairwise identity of at least 95%, at least 96%, at least 97%, at least 98%, at least 99%, or 100% as measured by the Jaccard similarity coefficient. In certain embodiments, the polyploid seed (e.g., the subpopulation of genetically uniform seed) comprises four or more haplotypes of the same or related species of plant. In some embodiments, germination of a seed of the population of polyploid seed or the subpopulation of genetically uniform polyploid seed results in a sterile plant that produces inviable gametes, seedless fruit, or a combination thereof. The population of polyploid seed may be from any plant species including, but not limited to, potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, dates, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussels sprouts, cabbage, collards, kale, lettuce, chard, spinach, bok choy, okra, cashew nuts, pineapple, celery, oat, birch, rapeseed, mustard, tea, hemp, safflower seed, cedar, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycress, green bean, bean, ground cherry, pine, pistachio nut, pea, turf grass, poplar, apricot, plum and prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.

In some embodiments, the polyploid seed (e.g., the subpopulation of genetically uniform seed) comprises one or more genetic modifications resulting in decreased expression of one or more, two or more, or three or more MiMe loci. The seed may comprise one or more genetic modifications resulting in decreased expression of MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH/DYAD, PS1, JASON, PC1, PC2, and FC. In some embodiments, the polyploid seed (e.g., the subpopulation of genetically uniform seed) comprises one or more genetic modifications resulting in altered activity of one or more, two or more, or three or more MiMe components. In some embodiments, the altered activity includes, for example, a dominant negative, constitutively active or null mutant of the one or more MiMe components. In one embodiment, the polyploid seed (e.g., the subpopulation of genetically uniform seed) comprises one or more genetic modifications resulting in decreased expression of REC8, SWITCH1/DYAD, or a combination thereof. In another embodiment, the polyploid seed (e.g., the subpopulation of genetically uniform seed) comprises one or more genetic modifications resulting in decreased expression of OSD1, CYCA1, TDM1, PC1, PC2, FC, or any combination thereof. In another embodiment, the polyploid seed (e.g., the subpopulation of genetically uniform seed) comprises one or more genetic modifications resulting in decreased expression of PAIR1, SPO11-1, SPO11-2, PRD1, PRD2, DFO, MTOPVIB, DSY1, SY1, SY2, SY3, SY4, or any combination thereof. In yet another embodiment, the polyploid seed (e.g., the subpopulation of genetically uniform seed) comprises one or more genetic modifications resulting in decreased expression of PS1, JASON, or a combination thereof. The polyploid seed may comprise genetic modifications in any combination of MiMe loci described herein. The one or more genetic modifications may include, but are not limited to, modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci.

In certain embodiments, the population of polyploid seed or the subpopulation of genetically uniform polyploid seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the population of polyploid or the subpopulation of genetically uniform polyploid seed has a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid seed or the subpopulation of genetically uniform polyploid seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, the population of polyploid seed or the subpopulation of genetically uniform polyploid seed has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In certain embodiments, the population of polyploid seed or the subpopulation of genetically uniform polyploid seed has (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the MiMe locus of the component of progression through the first division of meiosis of the third haplotype is PS1 or JASON. In still additional variations, the one or more MiMe loci of the component of progression through the second division of meiosis of the first and second haplotype comprise OSD1, CYCA1, TDM1, PC1, PC2, FC, or any combination thereof. In yet additional variations, the locus of the component of progression through the second division of meiosis of the third haplotype is OSD1, CYCA1, TDM1, PC1, PC2, or FC.

In certain embodiments, the population of polyploid seed or the subpopulation of genetically uniform polyploid seed has (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis of the first and second haplotype comprise PS1, JASON, or a combination thereof. In still additional variations, the MiMe locus of the component of progression through the first division of meiosis of the third haplotype is PS1 or JASON. In yet additional variations, the MiMe locus of the component of progression through the second division of meiosis of the third haplotype is OSD1, CYCA1, TDM1, PC1, PC2 or FC.

In some embodiments, the population of polyploid seed or the subpopulation of genetically uniform polyploid seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid seed or the subpopulation of genetically uniform polyploid seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the population of polyploid seed or the subpopulation of genetically uniform polyploid seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid seed or the subpopulation of genetically uniform polyploid seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid seed or the subpopulation of genetically uniform polyploid seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid seed or the subpopulation of genetically uniform polyploid seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the population of polyploid seed or the subpopulation of genetically uniform polyploid seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid seed or the subpopulation of genetically uniform polyploid seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid seed or the subpopulation of genetically uniform polyploid seed has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the population of polyploid seed or the subpopulation of genetically uniform polyploid seed has a complete MiMe genotype comprising (i) an os allele, wherein the subpopulation of genetically uniform polyploid seed is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) an os allele, wherein the subpopulation of genetically uniform polyploid seed is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid seed or the subpopulation of genetically uniform polyploid seed has a complete MiMe genotype comprising (i) a ps allele, wherein the subpopulation of genetically uniform polyploid seed is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) a ps allele, wherein the subpopulation of genetically uniform polyploid seed is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In another aspect, the present disclosure provides a population of polyploid seed having a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component, wherein at least 50% of the population of polyploid seed are genetically uniform, and wherein the population was obtained from a single plant or a set of plants such as, for example, a set of F1 hybrids. In some embodiments, the present disclosure provides a population of polyploid seed comprising a subpopulation of genetically uniform polyploid seed in an amount of at least 50% of the total number of seeds, the genetically uniform polyploid seed comprising a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component, and wherein the population was obtained from a single plant or a set of plants such as, for example, a set of F1 hybrids. The polyploid seed (e.g., the subpopulation of genetically uniform seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid seed are genetically uniform. In certain embodiments, the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid seed comprises the subpopulation of genetically uniform polyploid seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid seed has a pairwise identity of at least 95%, at least 96%, at least 97%, at least 98%, at least 99%, or 100% as measured by the Jaccard similarity coefficient. In some embodiments, the partially-complemented genotype comprises only MiMe alleles at one or more MiMe loci of a third MiMe component. In other embodiments, the partially-complemented genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In certain embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In another aspect, the present disclosure provides a population of polyploid seed having a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein at least 50% of the population of polyploid seed are genetically uniform, and wherein the population was obtained from a single parent plant or a set of plants, such as, for example, a set of F1 hybrids. In some embodiments, the present disclosure provides a population of polyploid seed comprising a subpopulation of genetically uniform polyploid seed in an amount of at least 50% of the total number of seeds, the genetically uniform polyploid seed comprising a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, and wherein the population was obtained from a single parent plant or a set of plants, such as, for example, a set of F1 hybrids. The polyploid seed (e.g., the subpopulation of genetically uniform seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid seed are genetically uniform. In certain embodiments, the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid seed comprises the subpopulation of genetically uniform polyploid seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid seed has a pairwise identity of at least 95%, at least 96%, at least 97%, at least 98%, at least 99%, or 100% as measured by the Jaccard similarity coefficient. In some embodiments, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

In another aspect, the present disclosure provides a population of polyploid seed having a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein at least 50% of the population of polyploid seed are genetically uniform, and wherein the population was obtained from a single parent plant or a set of plants, such as, for example, a set of F1 hybrids. In some embodiments, the present disclosure provides a population of polyploid seed comprising a subpopulation of genetically uniform polyploid seed in an amount of at least 50% of the total number of seeds, the genetically uniform polyploid seed comprising a partially-complemented Mime genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more Mime loci of a third Mime component; and (d) one or more MiMe alleles and one or more non-MiMe alleles at one or more MiMe loci of a fourth MiMe component, and wherein the population was obtained from a single parent plant or a set of plants, such as, for example, a set of F1 hybrids. The polyploid seed (e.g., the subpopulation of genetically uniform seed) may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid seed are genetically uniform. In certain embodiments, the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient. In certain embodiments, the population of polyploid seed comprises the subpopulation of genetically uniform polyploid seed in an amount of at least 70%, at least 90%, at least 95%, at least 96%, at least 97%, at least 98%, or at least 99% of the total number of seeds. In certain embodiments, each pair of seeds in the subpopulation of genetically uniform polyploid seed has a pairwise identity of at least 95%, at least 96%, at least 97%, at least 98%, at least 99%, or 100% as measured by the Jaccard similarity coefficient. In some embodiments, the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

In some embodiments of the foregoing aspects, the first MiMe component is a component of sister chromatid cohesion during the first division of meiosis. In some variations, the one or more MiMe loci of the first MiMe component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1. In some embodiments, the polyploid seed (e.g., the subpopulation of genetically uniform polyploid seed) comprises one or more genetic modifications resulting in altered activity of one or more, two or more, or three or more MiMe components. In some embodiments, the altered activity includes, for example, a dominant negative, constitutively active or null mutant of the one or more MiMe components

In some embodiments of the foregoing aspects, the population of polyploid seed or the subpopulation of genetically uniform polyploid seed has a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1.

In some embodiments of the foregoing aspects, the population of polyploid seed or the subpopulation of genetically uniform polyploid seed has a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only Mime alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of Spoil-1.

In some embodiments of the foregoing aspects, the population of polyploid seed or the subpopulation of genetically uniform polyploid seed has a partially complemented MiMe genotype comprising (i) only MiMe alleles at one or more TDM1 loci, wherein each of the Mime alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the Mime alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments of the foregoing aspects, the population of polyploid seed or the subpopulation of genetically uniform polyploid seed has a partially complemented MiMe genotype comprising (i) a ps allele, wherein the subpopulation of genetically uniform polyploid seed is heterozygous for the ps allele, (ii) an os allele, wherein the subpopulation of genetically uniform polyploid seed is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1.

In some embodiments of the foregoing aspects, the population of polyploid seed or the subpopulation of genetically uniform polyploid seed has a partially complemented MiMe genotype comprising (i) an os allele, wherein the subpopulation of genetically uniform polyploid seed is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.

In another aspect, the present disclosure provides a method of producing a population of polyploid seed described herein, the method comprising: (a) providing clonal gametes from a pair of parent MiMe plants that together comprise three or more haplotypes; and (b) crossing the clonal gametes to produce the population of polyploid seed. In some embodiments, at least 50% of the population of polyploid seed are genetically uniform and comprise three or more haplotypes. In some embodiments, at least 70%, at least 90%, at least 95%, at least 96%, at least 97%, at least 98%, or at least 99% of the population of polyploid seed produced by said method are genetically uniform. In some embodiments, the population of polyploid seed comprises a subpopulation of genetically uniform seed in an amount of at least 50% of the total number of seeds, the subpopulation of genetically uniform seed comprising three or more haplotypes. In certain embodiments, the population of polyploid seed comprises a subpopulation of genetically uniform seed in an amount of at least 70%, at least 90%, at least 95%, at least 96%, at least 97%, at least 98%, or at least 99% of the total number of seeds. In certain embodiments, the polyploid seed (e.g., the subpopulation of genetically uniform polyploid seed) produced by said method comprises four or more haplotypes of the same or related species of plant. The population of polyploid seed or the subpopulation of genetically uniform polyploid seed may be, for example, triploid, tetraploid, pentaploid, hexaploid, heptaploid, or octoploid. In some embodiments, germination of a seed of the population of polyploid seed produced by said method (e.g., germination of a seed of the subpopulation of genetically uniform polyploid seed) results in a sterile plant that produces inviable gametes, seedless fruit, or a combination thereof. The method may be used to produce a population of polyploid seed that may be from any plant species including, but not limited to, potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, dates, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussels sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine and mandarin, clementine, coffee, cola, hazelnut, saffron, melon and cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycress, green bean, bean, ground cherry, pine, pistachio nut, pea, turf grass, poplar, apricot, plum and prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.

In some embodiments of the method of producing the population of polyploid seeds comprises producing a population of polyploid seed comprising one or more genetic modifications resulting in decreased expression of one or more, two or more, or three or more MiMe loci. The population of polyploid seed or the subpopulation of genetically uniform polyploid seed may comprise one or more genetic modifications resulting in decreased expression of MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the polyploid seed produced by said method, or the subpopulation of genetically uniform polyploid seed, comprises one or more genetic modifications resulting in decreased expression of REC8, SWITCH1/DYAD, or a combination thereof. In another embodiment, the polyploid seed produced by said method, or the subpopulation of genetically uniform polyploid seed, comprises one or more genetic modifications resulting in decreased expression of OSD1, CYCA1, TDM1, PC1, PC2, FC, or a combination thereof. In another embodiment, the polyploid seed produced by said method, or the subpopulation of genetically uniform polyploid seed, comprises one or more genetic modifications resulting in decreased expression of PAIR1, SPO11-1, SPO11-2, PRD1, PRD2, DFO, MTOPVIB, DSY1, SY1, SY2, SY3, SY4, or a combination thereof. In yet another embodiment, the polyploid seed produced by said method comprises one or more genetic modifications resulting in decreased expression of PS1, JASON, or a combination thereof. The polyploid seed produced by said method, or the subpopulation of genetically uniform polyploid seed, may comprise genetic modifications in any combination of MiMe loci described herein. The genetic modifications may include, but are not limited to, modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci.

In certain embodiments, the population of polyploid seed produced by said method, or the subpopulation of genetically uniform polyploid seed, comprises a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the population of polyploid seed produced by said method, or the subpopulation of genetically uniform polyploid seed, comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid seed produced by said method, or the subpopulation of genetically uniform polyploid seed, has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, the population of polyploid seed produced by said method, or the subpopulation of genetically uniform polyploid seed, has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In another aspect, the present disclosure provides a method of producing a population of polyploid seed described herein having a partially-complemented MiMe genotype, the method comprising (a) providing clonal gametes from a first parent MiMe plant, wherein the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (b) providing clonal gametes from a second parent MiMe plant, wherein the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; and (c) crossing the clonal gametes from the first and second parent MiMe plants to produce the population of polyploid seed having a partially-complemented MiMe genotype. In some embodiments, at least 50% of the population of polyploid seed are genetically uniform and comprise two, three, or more haplotypes. In some embodiments, the population of polyploid seed comprises a subpopulation of genetically uniform seed in an amount of at least 50% of the total number of seeds, the subpopulation of genetically uniform seed comprising the partially-complemented MiMe genotype. In certain embodiments, at least one of the MiMe loci having only MiMe alleles of the first MiMe component of the first parent MiMe plant is the same as at least one of the MiMe loci having only MiMe alleles of the first MiMe component of the second parent MiMe plant. In some embodiments, at least one of the MiMe loci having only MiMe alleles of the third MiMe component of the first parent MiMe plant is the same as at least one of the MiMe loci having only MiMe alleles of the third MiMe component of the second parent MiMe plant. In other embodiments, the one or more MiMe loci having only MiMe alleles of the third MiMe component of the first parent MiMe plant are distinct from the one or more MiMe loci having only MiMe alleles of the third MiMe component of the second parent MiMe plant. In some embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In another aspect, the present disclosure provides a method of producing a population of polyploid seed having a partially-complemented MiMe genotype described herein, the method comprising (a) providing clonal gametes from a first parent MiMe plant, wherein the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; (b) providing clonal gametes from a second parent MiMe plant, wherein the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component; and (c) crossing the clonal gametes from the first and second parent MiMe plants to produce the population of polyploid seed having a partially-complemented MiMe genotype. In some embodiments, at least 50% of the population of polyploid seed are genetically uniform and comprise two, three, or more haplotypes. In some embodiments, the population of polyploid seed comprises a subpopulation of genetically uniform seed in an amount of at least 50% of the total number of seeds, the subpopulation of genetically uniform seed comprising the partially-complemented MiMe genotype. In some embodiments at least one of the MiMe loci having only MiMe alleles of the first MiMe component of the first parent MiMe plant is the same as at least one of the MiMe loci having only MiMe alleles of the first MiMe component of the second parent MiMe plant. In certain embodiments, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

In yet another aspect, the present disclosure provides a method of producing a population of polyploid seed having a partially-complemented MiMe genotype described herein, the method comprising (a) providing clonal gametes from a first parent MiMe plant, wherein the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination; (b) providing clonal gametes from a second parent MiMe plant, wherein the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and (c) crossing the clonal gametes from the first and second parent MiMe plants to produce the population of polyploid seed having a partially-complemented MiMe genotype. In some embodiments, at least 50% of the population of polyploid seed are genetically uniform and comprise two, three, or more haplotypes. In some embodiments, the population of polyploid seed comprises a subpopulation of genetically uniform seed in an amount of at least 50% of the total number of seeds, the subpopulation of genetically uniform seed comprising the partially-complemented MiMe genotype. In some embodiments, at least one of the MiMe loci having only MiMe alleles of the first MiMe component of the first parent MiMe plant is the same as at least one of the MiMe loci having only MiMe alleles of the first MiMe component of the second parent MiMe plant. In certain embodiments, the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

In some embodiments of the foregoing methods, the first MiMe component is a component of sister chromatid cohesion during the first division of meiosis. In some variations of said method, the MiMe loci of the first MiMe component of both the first and second parent MiMe plants comprise REC8. In certain embodiments of said method, the second MiMe component is a component of DNA double strand breakage during meiotic recombination. In some variations of said method, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation of said method, the first MiMe locus of the second MiMe component is PAIR1 and the second MiMe locus of the second MiMe component is SPO11-1. In further embodiments of said method, the third MiMe component is a component of progression through the second division of meiosis. In some embodiments of said method, at least one of the MiMe loci having only MiMe alleles of the third MiMe component of the first parent MiMe plant is the same as at least one of the MiMe loci having only MiMe alleles of the third MiMe component of the second parent MiMe plant. In some variations, the MiMe loci having only MiMe alleles of the component of progression through the second division of meiosis comprise OSD1, CYCA1, TDM1, PC1, PC2, FC, or any combination thereof. In one variation, the MiMe locus having only MiMe alleles of the third MiMe component is OSD1. In other embodiments of said method, the one or more MiMe loci having only MiMe alleles of the third MiMe component of the first parent MiMe plant are distinct from the one or more MiMe loci having only MiMe alleles of the third MiMe component of the second parent MiMe plant. In some variations, the MiMe loci having only MiMe alleles of the third MiMe component comprise OSD1, CYCA1, TDM1, PC1, PC2, FC, or any combination thereof. In one variation of said method, the MiMe loci having only MiMe alleles of the first MiMe component comprise REC8, the first MiMe locus of the second MiMe component is PAIR1, the second MiMe locus of the second MiMe component is SPO11-1, and the MiMe loci having only MiMe alleles of the third MiMe component comprise OSD1.

In another aspect, the present disclosure provides a method of breeding a polyploid hybrid plant line, comprising: (a) obtaining a set of lines of a plant; (b) breeding the lines using traditional plant breeding methods to produce a set of candidate lines of the plant; (c) selecting two or more candidate lines together comprising three or more haplotypes; (d) generating two parent MiMe plants from the two or more candidate lines that together comprise the three or more haplotypes; (e) providing clonal gametes from each of the parent MiMe plants; (f) crossing the clonal gametes to produce a hybrid polyploid seed comprising the three or more haplotypes; (g) growing the hybrid polyploid seed to produce a hybrid polyploid plant comprising three or more haplotypes; and (h) evaluating one or more characteristics of the hybrid polyploid plant. The candidate lines and the parent MiMe plants may be any ploidy, including, but not limited to, haploid, monoploid, diploid, triploid, or tetraploid. In additional embodiments of said breeding method, the hybrid polyploid plant is tetraploid, pentaploid, hexaploid, heptaploid, or octoploid.

In some embodiments of said breeding method, step (d) comprises introducing a complete MiMe genotype directly into two candidate lines to produce the two parent MiMe plants. In other embodiments of said breeding method, step (d) comprises introducing a partial MiMe genotype into two candidate lines to produce two grandparent non-MiMe plants each having a partial MiMe genotype, crossing said grandparent non-MiMe plants each having a partial MiMe genotype to produce the first parent MiMe plant, and introducing a complete MiMe genotype directly into a third candidate line to produce the second parent MiMe plant. In yet other embodiments of said breeding method, step (d) comprises introducing a partial MiMe genotype into four candidate lines to produce four grandparent non-MiMe plants each having a partial MiMe genotype, and crossing pairs of said grandparent non-MiMe plants each having a partial MiMe genotype to produce the two parent MiMe plants. In certain embodiments, step (d) further comprises propagating parent MiMe plants to scale production of homogenous seed.

In some embodiments of said breeding method, the parent MiMe plants of step (d) each have a complete MiMe genotype comprising MiMe alleles that are naturally-occurring, introduced via genetic modification, or a combination thereof. In certain embodiments, the genetic modifications result in decreased expression of one or more, two or more, or three or more MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the genetic modifications result in decreased expression of REC8, SWITCH1/DYAD, or a combination thereof. In another embodiment, the genetic modifications result in decreased expression of OSD1, CYCA1, TDM1, PC1, PC2, FC, or any combination thereof. In another embodiment, the genetic modifications result in decreased expression of PAIR1, SPO11-1, SPO11-2, PRD1, PRD2, DFO, MTOPVIB, DSY1, SY1, SY2, SY3, SY4, or any combination thereof. In yet another embodiment, the genetic modifications result in decreased expression of PS1, JASON, or a combination thereof. In some embodiments, the one or more genetic modifications are introduced by gene editing, transgenesis, or a combination thereof. The genetic modifications may be achieved by any methods described herein, including, but not limited to, gene disruption, gene knockout, gene knockdown, gene silencing, RNA interference, induction of methylation, or any combination thereof.

In certain embodiments, the population of polyploid seed produced by said breeding method comprises a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the population of polyploid seed produced by said breeding method comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid seed produced by said breeding method has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, the population of polyploid seed produced by said breeding method has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In certain embodiments of said breeding method, the method of breeding a population of polyploid seed further comprises (i) repeating steps (b)-(h) or steps (c)-(h) using the one or more characteristics of the hybrid polyploid plant evaluated in step (h) to guide the breeding of lines of step (b), the selecting of candidate lines of step (c), or both. In some variations, the one or more characteristics includes the heterotic performance of the three or more haplotypes of the polyploid hybrid plant evaluated in step (h).

In some embodiments of said breeding method, the set of lines in step (a) are obtained from one or more of natural diversity, existing breeding programs, or dihaploid induction of polyploid lines. In certain embodiments of said breeding method, step (a) further comprises organizing the set of lines into three or more heterotic groups, wherein each heterotic group comprises a haplotype, and wherein the haplotypes are grouped based on observed or predicted heterotic performance when combined in the hybrid polyploid plant of step (g). In one variation, step (a) comprises organizing the set of lines into four or more heterotic groups. In certain embodiments, heterotic performance is predicted via genome prediction modeling. In some embodiments of said breeding method, step (b) comprises reciprocal recurrent selection, inbreeding one or more of the plant lines to homozygosity, production of a doubled haploid line (e.g., a doubled monoploid line), backcrossing, or any other method known in the art for creating plant lines with high degrees of homozygosity, or a combination thereof. The candidate lines of step (c) may be inbred lines, hybrid lines, or a combination thereof.

In another aspect, the present disclosure provides a method of producing a population of polyploid seed comprising: (a) providing clonal gametes from a pair of parent MiMe plants that together comprise three or more haplotypes that were selected using the methods of breeding described herein based upon the polyploid plant comprising said three or more haplotypes having one or more desired characteristics; and (b) crossing the clonal gametes to produce the population of polyploid seed, wherein at least 50% of the population of polyploid seed are genetically uniform and comprise three or more haplotypes. The polyploid seed produced by said method may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid seed produced by said method are genetically uniform. In certain embodiments, the polyploid seed produced by said method comprises four or more haplotypes of the same or related species of plant. In some variations of said method, lines of the plant are maintained via vegetative propagation, selfing, apomixis, cell culture, or any combination thereof. In some embodiments, said method further comprises maintaining an inventory of lines of the plant from which haplotypes may be selected for rapid deterministic stacking of the haplotypes. In some variations, the inventory of lines comprises one or more plant lines having a complete MiMe genotype that is maintained through vegetative propagation, hybridization with a haploid inducer, or a combination thereof. In additional variations, the inventory of lines comprises one or more plant lines having a partial MiMe genotype.

In another aspect, the present disclosure provides a method of producing a population of polyploid seed comprising: (a) providing clonal gametes from a parent MiMe plant; (b) providing haploid (e.g., monoploid) gametes from a homozygous parent non-MiMe plant; and (c) crossing the clonal gametes with the haploid (e.g., monoploid) gametes to produce the population of polyploid seed, wherein the clonal gametes and the haploid (e.g., monoploid) gametes together comprise three or more haplotypes, and wherein at least 50% of the population of polyploid seed are genetically uniform and comprise three or more haplotypes. The parent MiMe plant may be, for example, diploid, triploid, or tetraploid. The homozygous parent non-MiMe plant may be, for example, diploid or tetraploid. The polyploid seed produced by said method may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid seed produced by said method are genetically uniform. In certain embodiments, the polyploid seed produced by said method comprises four or more haplotypes of the same or related species of plant. In some embodiments, germination of a seed of the population of polyploid seed produced by said method results in a sterile plant that produces inviable gametes, seedless fruit, or a combination thereof. The method may be used to produce a population of polyploid seed that may be from any plant species including, but not limited to, potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, dates, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussels sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine and mandarin, clementine, coffee, cola, hazelnut, saffron, melon and cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycress, green bean, bean, ground cherry, pine, pistachio nut, pea, turf grass, poplar, apricot, plum and prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.

In some embodiments, the method of producing a population of polyploid seed comprises producing a population of polyploid seed comprising one or more genetic modifications resulting in decreased expression of one or more, two or more, or three or more MiMe loci. The seed may comprise one or more genetic modifications resulting in decreased expression of MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the polyploid seed produced by said method comprises one or more genetic modifications resulting in decreased expression of REC8, SWITCH1/DYAD, or a combination thereof. In another embodiment, the polyploid seed produced by said method comprises one or more genetic modifications resulting in decreased expression of OSD1, CYCA1, TDM1, PC1, PC2, FC, or a combination thereof. In another embodiment, the polyploid seed produced by said method comprises one or more genetic modifications resulting in decreased expression of PAIR1, SPO11-1, SPO11-2, PRD1, PRD2, DFO, MTOPVIB, DSY1, SY1, SY2, SY3, SY4, or a combination thereof. In yet another embodiment, the polyploid seed produced by said method comprises one or more genetic modifications resulting in decreased expression of PS1, JASON, or a combination thereof. The polyploid seed produced by said method may comprise genetic modifications in any combination of MiMe loci described herein. The genetic modifications may include, but are not limited to, modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci.

In certain embodiments, the population of polyploid seed produced by said method comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid seed produced by said method has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In another aspect, the present disclosure provides a method of breeding a polyploid hybrid plant line, comprising: (a) obtaining a set of lines of a plant; (b) breeding the lines using traditional plant breeding methods to produce a set of candidate lines of the plant; (c) selecting two or more candidate lines together comprising three or more haplotypes; (d) generating a parent MiMe plant from one of the two or more candidate lines; (e) providing clonal gametes from the parent MiMe plant; (f) providing haploid (e.g., monoploid) gametes from a homozygous parent non-MiMe plant of one of the two or more candidate lines; (g) crossing the clonal gametes with the haploid (e.g., monoploid) gametes to produce a hybrid polyploid seed; (h) growing the hybrid polyploid seed to produce a hybrid polyploid plant; and (i) evaluating one or more characteristics of the hybrid polyploid plant, wherein the parent MiMe plant and the homozygous parent non-MiMe plant together comprise three or more haplotypes, wherein the crossing of step (g) results in the hybrid polyploid seed comprising three or more haplotypes, and wherein the growing of step (h) results in the hybrid polyploid plant comprising three or more haplotypes. The candidate lines, parent MiMe plant, and the homozygous parent non-MiMe plant may be any ploidy, including, but not limited to, haploid, monoploid, diploid, triploid, or tetraploid. In some embodiments of said breeding method, the hybrid polyploid plant is tetraploid, pentaploid, hexaploid, heptaploid, or octoploid.

In some embodiments of said breeding method, step (d) comprises introducing a complete MiMe genotype directly into a candidate line to produce the parent MiMe plant. In further embodiments of said breeding method, step (d) comprises introducing a partial MiMe genotype into two candidate lines to produce two grandparent non-MiMe plants each having a partial MiMe genotype, crossing said grandparent non-MiMe plants each having a partial MiMe genotype to produce the parent MiMe plant. In certain embodiments, step (d) further comprises propagating the parent MiMe plant to scale production of homogenous seed.

In some embodiments of said breeding method, the parent MiMe plant of step (d) has a complete MiMe genotype comprising MiMe alleles that are naturally-occurring, introduced via genetic modification, or a combination thereof. In certain embodiments, the genetic modifications result in decreased expression of one or more, two or more, or three or more MiMe loci including, but not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one embodiment, the genetic modifications result in decreased expression of REC8, SWITCH1/DYAD, or a combination thereof. In another embodiment, the genetic modifications result in decreased expression of OSD1, CYCA1, TDM1, PC1, PC2, FC, or any combination thereof. In another embodiment, the genetic modifications result in decreased expression of PAIR1, SPO11-1, SPO11-2, PRD1, PRD2, DFO, MTOPVIB, DSY1, SY1, SY2, SY3, SY4, or any combination thereof. In yet another embodiment, the genetic modifications result in decreased expression of PS1, JASON, or a combination thereof. In some embodiments, the one or more genetic modifications are introduced by gene editing, transgenesis, or a combination thereof. The genetic modifications may be achieved by any methods described herein, including, but not limited to, gene disruption, gene knockout, gene knockdown, gene silencing, RNA interference, induction of methylation, or any combination thereof.

In certain embodiments, the population of polyploid seed produced by said breeding method comprises a partial MiMe genotype comprising (a) one or more MiMe alleles conferring decreased expression of one or more MiMe loci of each of the first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof.

In certain embodiments, the population of polyploid seed produced by said breeding method has a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis comprise PS1, JASON, or a combination thereof.

In certain embodiments of said breeding method, the method of breeding a population of polyploid seed further comprises (j) repeating steps (b)-(i) or steps (c)-(i) using the one or more characteristics of the hybrid polyploid plant evaluated in step (i) to guide the breeding of lines of step (b), the selecting of candidate lines of step (c), or both. In some variations, the one or more characteristics includes the heterotic performance of the three or more haplotypes of the polyploid hybrid plant evaluated in step (i).

In some embodiments of said breeding method, the set of lines in step (a) are obtained from one or more of natural diversity, existing breeding programs, or dihaploid induction of polyploid lines. In certain embodiments of said breeding method, step (a) further comprises organizing the set of lines into three or more heterotic groups, wherein each heterotic group comprises a haplotype, and wherein the haplotypes are grouped based on observed or predicted heterotic performance when combined in the hybrid polyploid plant of step (h). In one variation, step (a) comprises organizing the set of lines into four or more heterotic groups. In certain embodiments, heterotic performance is predicted via genome prediction modeling. In some embodiments of said breeding method, step (b) comprises reciprocal recurrent selection, inbreeding one or more of the plant lines to homozygosity, production of a doubled haploid line (e.g., a doubled monoploid line), backcrossing, or any other method known in the art for creating plant lines with high degrees of homozygosity, or a combination thereof. The candidate lines of step (c) may be inbred lines, hybrid lines, or a combination thereof.

In another aspect, the present disclosure provides a method of producing a population of polyploid seed comprising: (a) selecting three or more haplotypes using the method of breeding described herein based upon the polyploid plant comprising said three or more haplotypes having one or more desired characteristics; (b) providing clonal gametes from a parent MiMe plant; (c) providing haploid (e.g., monoploid) gametes from a homozygous parent non-MiMe plant; (d) crossing the clonal gametes with the haploid (e.g., monoploid) gametes to produce the population of polyploid seed; wherein the parent MiMe plant and the homozygous parent non-MiMe plant together comprise the three or more haplotypes selected in step (a), wherein the crossing of step (d) results in a population of polyploid seed comprising the three or more haplotypes selected in step (a), and wherein at least 50% of the population of polyploid seed are genetically uniform and comprise three or more haplotypes. The polyploid seed produced by said method may be, for example, triploid, tetraploid, pentaploid, hexaploid, heptaploid, or octoploid. In some embodiments, at least 70%, at least 90%, at least 95%, at least 96%, at least 97%, at least 98%, or at least 99% of the population of polyploid seed produced by said method are genetically uniform. In certain embodiments, the polyploid seed produced by said method comprises four or more haplotypes of the same or related species of plant. In some variations of said method, lines of the plant are maintained via vegetative propagation, selfing, apomixis, cell culture, or any combination thereof. In some embodiments, said method further comprises maintaining an inventory of lines of the plant from which haplotypes may be selected for rapid deterministic stacking of the haplotypes. In some variations, the inventory of lines comprises one or more plant lines having a complete MiMe genotype that is maintained through vegetative propagation, hybridization with a haploid inducer, or a combination thereof. In additional variations, the inventory of lines comprises one or more plant lines having a partial MiMe genotype.

In another aspect, the present disclosure provides a method of breeding a polyploid plant, comprising (a) obtaining a set of lines of a plant; (b) breeding the lines using traditional plant breeding methods to produce a set of candidate lines of the plant; (c) selecting one or more candidate lines; (d) generating a first parent MiMe plant from one of the candidate lines, wherein the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (e) generating a second parent MiMe plant from one of the candidate lines, wherein the second parent MiMe plant has only MiMe alleles at one or more MiMe loci of the first MiMe component, only non-MiMe alleles at the first Mime locus of the second MiMe component, only MiMe alleles at the second MiMe locus of the second MiMe component, and only MiMe alleles at one or more MiMe loci of the third MiMe component, wherein at least one of the MiMe loci having only MiMe alleles of the first MiMe component of the first parent MiMe plant is the same as at least one of the MiMe loci having only MiMe alleles of the first MiMe component of the second parent MiMe plant; (f) providing clonal gametes from each of the parent MiMe plants; (g) crossing the clonal gametes to produce a polyploid seed; (h) growing the polyploid seed to produce a polyploid plant; and (i) evaluating one or more characteristics of the polyploid plant. In some embodiments, at least one of the MiMe loci having only MiMe alleles of the third MiMe component of the first parent MiMe plant is the same as at least one of the MiMe loci having only MiMe alleles of the third MiMe component of the second parent MiMe plant. In other embodiments, the one or more MiMe loci having only MiMe alleles of the third MiMe component of the first parent MiMe plant are distinct from the one or more MiMe loci having only MiMe alleles of the third MiMe component of the second parent MiMe plant. In certain embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In some embodiments of said breeding method, the first MiMe component is a component of sister chromatid cohesion during the first division of meiosis. In some variations of said breeding method, the MiMe loci of the first MiMe component of both the first and second parent MiMe plants comprise REC8. In certain embodiments of said breeding method, the second MiMe component is a component of DNA double strand breakage during meiotic recombination. In some variations of said breeding method, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation of said breeding method, the first MiMe locus of the second MiMe component is PAIR1 and the second MiMe locus of the second MiMe component is SPO11-1. In further embodiments of said breeding method, the third MiMe component is a component of progression through the second division of meiosis. In some embodiments of said breeding method, at least one of the MiMe loci having only MiMe alleles of the third MiMe component of the first parent MiMe plant is the same as at least one of the MiMe loci having only MiMe alleles of the third MiMe component of the second parent MiMe plant. In some variations, the MiMe loci having only MiMe alleles of the component of progression through the second division of meiosis comprise OSD1, CYCA1, TDM1, PC1, PC2, FC, or any combination thereof. In one variation, the MiMe locus having only MiMe alleles of the third MiMe component is OSD1. In other embodiments of said breeding method, the one or more MiMe loci having only MiMe alleles of the third MiMe component of the first parent MiMe plant are distinct from the one or more MiMe loci having only MiMe alleles of the third MiMe component of the second parent MiMe plant. In some variations, the MiMe loci having only MiMe alleles of the third MiMe component comprise OSD1, CYCA1, TDM1, PC1, PC2, FC, or any combination thereof. In one variation of said breeding method, the MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1.

In another aspect, the present disclosure provides a method of producing a population of polyploid seed comprising (a) providing clonal gametes from a pair of parent MiMe plants together comprising two or more haplotypes that were selected using the foregoing method of breeding based upon the polyploid plant comprising said two or more haplotypes having one or more desired characteristics, wherein: (i) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (ii) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; and (iii) at least one of the MiMe loci having only MiMe alleles of the first MiMe component of the first parent MiMe plant is the same as at least one of the MiMe loci having only MiMe alleles of the first MiMe component of the second parent MiMe plant; and (b) crossing the clonal gametes to produce the population of polyploid seed, wherein at least 50% of the population of polyploid seed are genetically uniform and comprise two or more haplotypes. In some embodiments, at least one of the MiMe loci having only MiMe alleles of the third MiMe component of the first parent MiMe plant is the same as at least one of the MiMe loci having only MiMe alleles of the third MiMe component of the second parent MiMe plant. In other embodiments, the one or more MiMe loci having only MiMe alleles of the third MiMe component of the first parent MiMe plant are distinct from the one or more MiMe loci having only MiMe alleles of the third MiMe component of the second parent MiMe plant.

In another aspect, the present disclosure provides a method of breeding a polyploid plant, comprising (a) obtaining a set of lines of a plant; (b) breeding the lines using traditional plant breeding methods to produce a set of candidate lines of the plant; (c) selecting one or more candidate lines; (d) generating a first parent MiMe plant from one of the candidate lines, wherein the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; (e) generating a second parent MiMe plant from one of the candidate lines, wherein the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component, and at least one of the MiMe loci having only MiMe alleles of the first MiMe component of the first parent MiMe plant is the same as at least one of the MiMe loci having only MiMe alleles of the first MiMe component of the second parent MiMe plant; (f) providing clonal gametes from each of the parent MiMe plants; (g) crossing the clonal gametes to produce a polyploid seed; (h) growing the polyploid seed to produce a polyploid plant; and (i) evaluating one or more characteristics of the polyploid plant. In some embodiments of said breeding method, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.

In another aspect, the present disclosure provides a method of producing a population of polyploid seed comprising (a) providing clonal gametes from a pair of parent MiMe plants together comprising two or more haplotypes that were selected using the foregoing method of breeding based upon the polyploid plant comprising said two or more haplotypes having one or more desired characteristics, wherein: (i) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; (ii) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component; and (iii) at least one of the MiMe loci having only MiMe alleles of the first MiMe component of the first parent MiMe plant is the same as at least one of the MiMe loci having only MiMe alleles of the first MiMe component of the second parent MiMe plant; and (b) crossing the clonal gametes to produce the population of polyploid seed wherein at least 50% of the population of polyploid seed are genetically uniform and comprise two or more haplotypes.

In another aspect, the present disclosure provides a method of breeding a polyploid plant, comprising (a) obtaining a set of lines of a plant; (b) breeding the lines using traditional plant breeding methods to produce a set of candidate lines of the plant; (c) selecting one or more candidate lines; (d) generating a first parent MiMe plant from one of the candidate lines, wherein the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first Mime component is a component of DNA double strand breakage during meiotic recombination; (e) generating a second parent MiMe plant from one of the candidate lines, wherein the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component, and at least one of the MiMe loci having only MiMe alleles of the first MiMe component of the first parent MiMe plant is the same as at least one of the MiMe loci having only MiMe alleles of the first MiMe component of the second parent MiMe plant; (f) providing clonal gametes from each of the parent MiMe plants; (g) crossing the clonal gametes to produce a polyploid seed; (h) growing the polyploid seed to produce a polyploid plant; and (i) evaluating one or more characteristics of the polyploid plant. In some embodiments of said breeding method, the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.

In another aspect, the present disclosure provides a method of producing a population of polyploid seed comprising (a) providing clonal gametes from a pair of parent MiMe plants together comprising two or more haplotypes that were selected using the foregoing method of breeding based upon the polyploid plant comprising said two or more haplotypes having one or more desired characteristics, wherein: (i) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; (ii) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component; and (iii) at least one of the MiMe loci having only MiMe alleles of the first MiMe component of the first parent MiMe plant is the same as at least one of the MiMe loci having only MiMe alleles of the first MiMe component of the second parent MiMe plant; and (b) crossing the clonal gametes to produce the population of polyploid seed, wherein at least 50% of the population of polyploid seed are genetically uniform and comprise two or more haplotypes.

In some embodiments of the foregoing breeding methods, steps (d) and (e) comprise introducing a complete MiMe genotype directly into two candidate lines to produce the two parent MiMe plants. In further embodiments of the foregoing breeding methods, steps (d) and (e) comprise introducing a partial MiMe genotype into two candidate lines to produce two grandparent non-MiMe plants each having a partial MiMe genotype, crossing said grandparent non-MiMe plants each having a partial MiMe genotype to produce the first parent MiMe plant, and introducing a complete MiMe genotype directly into a third candidate line to produce the second parent MiMe plant. In yet further embodiments of the foregoing breeding methods, steps (d) and (e) comprise introducing a partial MiMe genotype into four candidate lines to produce four grandparent non-MiMe plants each having a partial MiMe genotype, and crossing pairs of said grandparent non-MiMe plants each having a partial MiMe genotype to produce the two parent MiMe plants. In certain embodiments of the foregoing breeding methods, steps (d) and (e) further comprise propagating parent MiMe plants to scale production of homogenous seed.

In some embodiments of the foregoing breeding methods, the method further comprises ( ) repeating steps (b)-(i) or steps (c)-(i) using the one or more characteristics of the polyploid plant evaluated in step (i) to guide the breeding of the lines of step (b), the selecting of candidate lines of step (c), or both. In certain embodiments of the foregoing breeding methods, the one or more characteristics includes the heterotic performance of the two, three, four, or more haplotypes of the polyploid plant evaluated in step (i). In some embodiments of the foregoing breeding methods, the set of lines in step (a) are obtained from one or more of: natural diversity, existing breeding programs, or dihaploid induction of polyploid lines. In certain embodiments of the foregoing breeding methods, step (a) further comprises organizing the set of lines into two, three, four, or more heterotic groups, wherein each heterotic group comprises a haplotype, and wherein the haplotypes are grouped based on observed or predicted heterotic performance when combined in the polyploid plant of step (i). In some variations, heterotic performance is predicted via genome prediction modeling. In some embodiments of the foregoing breeding methods, step (b) comprises reciprocal recurrent selection, inbreeding one or more of the plant lines to homozygosity, production of a doubled haploid line (e.g., a doubled monoploid line), backcrossing, or any other method known in the art for creating plant lines with high degrees of homozygosity, or a combination thereof. In some embodiments of the foregoing breeding methods, one or more of the candidate lines of step (c) are inbred lines. In further embodiments of the foregoing breeding methods, one or more of the candidate lines of step (c) are hybrid lines.

In some embodiments of any of the forgoing breeding methods, the first and second parent MiMe plants together comprise two, three, four, or more haplotypes, resulting in a polyploid plant comprising two, three, four, or more haplotypes. In some embodiments of the foregoing breeding methods, the lines of the plant are maintained via vegetative propagation, selfing, apomixis, cell culture, or any combination thereof. In further embodiments of the forgoing methods, the method further comprises maintaining an inventory of lines of the plant from which haplotypes may be selected for rapid deterministic stacking of the haplotypes. In some variations, the inventory of lines comprises one or more plant lines having a complete MiMe genotype that is maintained through vegetative propagation, hybridization with a haploid inducer, or a combination thereof. In additional variations, the inventory of lines comprises one or more plant lines having a partial MiMe genotype.

In some embodiments of the foregoing aspects and embodiments, the one or more MiMe loci of the component of sister chromatid cohesion during the first division of meiosis comprise REC8, SWITCH1/DYAD, or a combination thereof. In some embodiments of the foregoing aspects and embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In some embodiments of the foregoing aspects and embodiments, the one or more MiMe loci of the component of progression through the second division of meiosis comprise OSD1, CYCA1, TDM1, PC1, PC2, FC, or any combination thereof. In some embodiments of the foregoing aspects and embodiments, the one or more MiMe loci of the component of progression through the first division of meiosis comprise PS1, JASON, or a combination thereof.

In some embodiments of any of the foregoing aspects and embodiments, the MiMe alleles comprise alleles that are naturally-occurring, introduced via genetic modification, or a combination thereof. In certain embodiments of the foregoing aspects and embodiments, the MiMe alleles comprise one or more genetic modifications. In further embodiments of the foregoing aspects and embodiments, one or more of the genetic modifications are at MiMe loci encoding gene products of the MiMe components. In some variations, the genetic modifications comprise modification of an enhancer in the MiMe loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci, or any combination thereof. In some embodiments of the foregoing aspects and embodiments, one or more genetic modifications are introduced by gene editing, transgenesis, or a combination thereof. In further embodiments of the foregoing aspects and embodiments, the decreased expression of the one or more MiMe loci is achieved by gene disruption, gene knockout, gene knockdown, gene silencing, RNA interference, induction of methylation, or any combination thereof.

In any of the foregoing aspects and embodiments, the population of polyploid seed may be, for example, triploid, tetraploid, pentaploid, hexaploid, heptaploid, or octoploid. In some embodiments of the foregoing aspects and embodiments, at least 50%, at least 70%, at least 80%, at least 90%, at least 95%, at least 96%, at least 97%, at least 98%, or at least 99% of the population of polyploid seed are genetically uniform. In some embodiments of the foregoing aspects and embodiments, the population of polyploid seed comprises a subpopulation of genetically uniform polyploid seed in an amount of at least 50%, at least at least 70%, at least 75%, at least 80%, at least 90%, at least 95%, at least 96%, at least 97%, at least 98%, or at least 99% of the total number of seeds. In certain embodiments of the foregoing aspects and embodiments, the polyploid seed (e.g., the subpopulation of genetically uniform polyploid seed) comprises two, three, four, or more haplotypes of the same or related species of plant. In some embodiments of the foregoing aspects and embodiments, germination of a seed of the population of polyploid seed, or a seed of the subpopulation of genetically uniform polyploid seed, results in a plant that produces inviable gametes, seedless fruit, or a combination thereof. In some embodiments, germination of a seed of the population of polyploid seed, or a seed of the subpopulation of genetically uniform polyploid seed, results in a seedless plant. In certain embodiments of the foregoing aspects and embodiments, the polyploid seed is from a parthenocarpic plant. In any of the foregoing aspects and embodiments, the population of polyploid seed may be from any plant species including, but not limited to, potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussels sprouts, cabbage, collards, kale, lettuce, chard, spinach, bok choy, basil, okra, fir, maple, apricot, plantain, birch, cedar, cherry, citron, clementine, Eucalyptus, ground cherry, hop, kenaf, larch, melon and cantaloupe, Miscanthus, nectarine, olive, switchgrass, peach, spruce, pine, pineapple, plum and prune, poplar, rubber rabbit brush, rubber tree, Russian dandelion, satsuma, sorghum, tangerine and mandarin, teff, hemlock tree, turf grass, kohlrabi, broccoli and cauliflower.

In another aspect, the present disclosure provides a genetically modified plant, plant part, or plant cell. In some embodiments, the genetically modified plant, plant part, or plant cell comprises: i) three or more haplotypes; and ii) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the genetically modified plant, plant part, or plant cell comprises: i) three or more haplotypes; and ii) a partial MiMe genotype comprising: (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations of the foregoing embodiments, the one or more MiMe loci of the component of sister chromatid cohesion during the first division of meiosis comprise REC8, SWITCH1/DYAD, or a combination thereof. In additional variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations of the foregoing embodiments, the one or more MiMe loci of the component of progression through the second division of meiosis comprise OSD1, CYCA1, TDM1, PC1, PC2, FC, or any combination thereof.

In some embodiments of said genetically modified plant, plant part, or plant cell, the present disclosure provides a genetically modified plant, plant part, or plant cell comprising: i) three or more haplotypes; and ii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments of this aspect, the genetically modified plant, plant part, or plant cell comprising: i) three or more haplotypes; and ii) a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of progression through the first division of meiosis comprise PS1, JASON, or a combination thereof.

In some embodiments of said genetically modified plant, plant part, or plant cell, the genetically modified plant, plant part, or plant cell comprises: (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations of the foregoing embodiments, the one or more MiMe loci of the component of sister chromatid cohesion during the first division of meiosis comprise REC8, SWITCH1/DYAD, or a combination thereof. In additional variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations of the foregoing embodiments, the MiMe locus of the component of progression through the first division of meiosis of the third haplotype is PS1 or JASON. In still additional variations of the foregoing embodiments, the one or more MiMe loci of the component of progression through the second division of meiosis of the first and second haplotype comprise OSD1, CYCA1, TDM1, PC1, PC2, FC, or any combination thereof. In yet additional variations of the foregoing embodiments, the locus of the component of progression through the second division of meiosis of the third haplotype is OSD1, CYCA1, TDM1, PC1, PC2, or FC.

In some embodiments of said genetically modified plant, plant part, or plant cell, the genetically modified plant, plant part, or plant cell comprises: (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of progression through the first division of meiosis of the first and second haplotype comprise PS1, JASON, or a combination thereof. In yet additional variations of the foregoing embodiments, the MiMe locus of the component of progression through the first division of meiosis of the third haplotype is PS1 or JASON. In still additional variations of the foregoing embodiments, the MiMe locus of the component of progression through the second division of meiosis of the third haplotype is OSD1, CYCA1, TDM1, PC1, PC2 or FC.

In some embodiments, the genetically modified plant, plant part, or plant cell has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the genetically modified plant, plant part, or plant cell has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the genetically modified plant, plant part, or plant cell has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the genetically modified plant, plant part, or plant cell has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the genetically modified plant, plant part, or plant cell has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1 In some embodiments, the genetically modified plant, plant part, or plant cell has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the genetically modified plant, plant part, or plant cell has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the genetically modified plant, plant part, or plant cell has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the genetically modified plant, plant part, or plant cell has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the genetically modified plant, plant part, or plant cell has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the genetically modified plant, plant part, or plant cell has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the genetically modified plant, plant part, or plant cell has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1.

In some embodiments, the genetically modified plant, plant part, or plant cell has a complete MiMe genotype comprising (i) an os allele, wherein the genetically modified plant, plant part, or plant cell is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the genetically modified plant, plant part, or plant cell has a partial MiMe genotype comprising (i) an os allele, wherein the genetically modified plant, plant part, or plant cell is heterozygous for the os allele, (ii) at least one Mime allele and at least one non-MiMe allele at one or more REC8 loci, wherein each of the Mime alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the genetically modified plant, plant part, or plant cell has a complete MiMe genotype comprising (i) a ps allele, wherein the genetically modified plant, plant part, or plant cell is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1. In some embodiments, the genetically modified plant, plant part, or plant cell has a partial Mime genotype comprising (i) a ps allele, wherein the genetically modified plant, plant part, or plant cell is heterozygous for the ps allele, and (ii) at least one Mime allele and at least one non-MiMe allele at one or more SPO11-1 loci, wherein each of the Mime alleles at the one or more SPO11-1 loci comprise one or more genetic modifications resulting in decreased expression of SPO11-1.

In some aspects, provided herein is a genetically modified plant, plant part, or plant cell having a partially-complemented MiMe genotype. In some embodiments, the genetically modified plant, plant part, or plant cell has a partially-complemented Mime genotype comprising: (a) only Mime alleles at one or more MiMe loci of a first Mime component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, the first MiMe component is a component of sister chromatid cohesion during the first division of meiosis. In some variations, the one or more Mime loci of the first MiMe component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1.

In some embodiments, the present disclosure provides a genetically modified plant, plant part, or plant cell having a partially-complemented MiMe genotype comprising: (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiment, the one or more MiMe loci of the component of progression through the first division of meiosis comprise PS1, JASON, or a combination thereof.

In some embodiments, the present disclosure provides a genetically modified plant, plant part, or plant cell having a partially-complemented MiMe genotype comprising: (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of sister chromatid cohesion during the first division of meiosis comprise REC8, SWITCH1/DYAD, or a combination thereof. In yet additional variations of the foregoing embodiments, the one or more MiMe loci of the component of progression through the second division of meiosis comprise OSD1, CYCA1, TDM1, PC1, PC2, FC, or any combination thereof. In still additional variations of the foregoing embodiments, the one or more MiMe loci of the component of progression through the first division of meiosis comprise PS1, JASON, or a combination thereof.

In some embodiments, the genetically modified plant, plant part, or plant cell has a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1.

In some embodiments, the genetically modified plant, plant part, or plant cell has a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments, the genetically modified plant, plant part, or plant cell has a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments, the genetically modified plant, plant part, or plant cell has a partially complemented MiMe genotype comprising (i) a ps allele, wherein the genetically modified plant, plant part, or plant cell is heterozygous for the ps allele, (ii) an os allele, wherein the genetically modified plant, plant part, or plant cell is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1.

In some embodiments, the genetically modified plant, plant part, or plant cell has a partially complemented MiMe genotype comprising (i) an os allele, wherein the genetically modified plant, plant part, or plant cell is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more comprise one or more genetic modifications resulting in decreased expression of SPO11-1 loci.

In some embodiments of said genetically modified plant, plant part, or plant cell, which may be combined with any of the preceding embodiments, the genetically modified plant, plant part, or plant cell is diploid, triploid, tetraploid, pentaploid, hexaploid, heptaploid, or octoploid. In additional embodiments which may be combined with any of the preceding embodiments, the genetically modified plant, plant part, or plant cell comprises two, three, four, or more haplotypes of the same or related species of plant. In yet additional embodiments which may be combined with any of the preceding embodiments, the genetically modified plant, plant part, or plant cell is from a plant selected from the group consisting of potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, dates, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussels sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine and mandarin, clementine, coffee, cola, hazelnut, saffron, melon and cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycress, green bean, bean, ground cherry, pine, pistachio nut, pea, turf grass, poplar, apricot, plum and prune, almond, nectarine, peach, cherry, rose, Rubus, rye, sesame, sorghum, spruce, switchgrass, Russian dandelion, cacao, durum wheat, spelt, wheat, broad bean, cowpea, ginger, plantain, kohlrabi, broccoli and cauliflower. In some embodiments which may be combined with the foregoing embodiments, the genetically modified plant, plant part, or plant cell is from a parthenocarpic plant. In certain embodiments, which may be combined with any of the preceding embodiments, the genetically modified plant part is a non-regenerable plant part. In certain embodiments, which may be combined with any of the preceding embodiments, the genetically modified plant cell is a non-regenerable plant cell. In certain embodiments, which may be combined with any of the preceding embodiments, the plant part is a flower, a pistil, a leaf, a stem, a petiole, a cutting, a tissue, a seed coat, an ovule, pollen, a tuber, a root, a rootstock, a scion, a fruit, a cotyledon, a hypocotyl, a protoplast, an embryo, an anther, or a portion thereof. In one variation of the foregoing embodiments, the genetically modified plant part is a seedless fruit.

In another aspect, provided herein is a processed plant product derived from any of the foregoing embodiments of genetically modified plants, plant parts, or plant cells, wherein the processed plant product comprises a detectable amount of the one or more MiMe alleles of the genetically modified plant, plant part, or plant cell. In some embodiments, the product is selected from the group consisting of plant biomass, oil, meal, food starch, syrup, animal feed, flour, flakes, bran, lint, hulls, processed seed, puree, juice, juice concentrate, pulp, pomace, preserve, and sauce. In certain embodiments, the processed plant product is non-regenerable.

### DESCRIPTION OF THE FIGURES

The present application can be understood by reference to the following description taken in conjunction with the accompanying figures.

FIG. 1 depicts a phylogenetic tree of REC8 protein sequences from dicotyledonous plants.

FIG. 2 depicts a phylogenetic tree of REC8 protein sequences from monocotyledonous plants.

FIG. 3 depicts a phylogenetic tree of SPO11-1 protein sequences from dicotyledonous plants.

FIG. 4 depicts a phylogenetic tree of SPO11-1 protein sequences from monocotyledonous plants.

FIG. 5 depicts a phylogenetic tree of PAIR1 protein sequences from dicotyledonous plants.

FIG. 6 depicts a phylogenetic tree of PAIR1 protein sequences from monocotyledonous plants.

FIG. 7 depicts a phylogenetic tree of OSD1 protein sequences from dicotyledonous plants.

FIG. 8 depicts a phylogenetic tree of OSD1 protein sequences from monocotyledonous plants.

FIG. 9 depicts a plasmid map of pLDB3, used to introduce MiMe gene edits in Zea mays.

FIG. 10 depicts a plasmid map of pLDB11B, used to introduce MiMe gene edits in Zea mays.

FIG. 11 depicts a plasmid map of pLDB12B, used to introduce MiMe gene edits in Zea mays.

FIG. 12 depicts a plasmid map of pMEM4, used to introduce MiMe gene edits in Zea mays.

FIG. 13 depicts a plasmid map of pMEM6b, used to introduce MiMe gene edits in Zea mays.

FIG. 14 depicts a plasmid map of pOGZ2, used to introduce MiMe gene edits in Zea mays.

FIG. 15 depicts a plasmid map of pOGZ4, used to introduce MiMe gene edits in Zea mays.

FIG. 16 illustrates an embodiment where homozygous gene editing occurs at the parent stage.

FIG. 17 illustrates an embodiment where grandparents are inbred and homozygous gene editing occurs at the parent stage.

FIG. 18 illustrates an embodiment where heterozygous gene editing occurs at the grandparent stage.

FIG. 19 illustrates an embodiment where grandparents are inbred and heterozygous gene editing at the grandparent stage.

FIG. 20 illustrates an embodiment where MiMe loci are propagated at the grandparent stage.

FIG. 21 illustrates an embodiment where grandparents are inbred and MiMe loci are propagated at the grandparent stage.

FIG. 22 illustrates an embodiment where MiMe loci are propagated at the great-grandparent stage.

FIG. 23 illustrates an embodiment where unilateral editing results in sexual tetraploidization.

FIGS. 24A-24C illustrate embodiments where sterility of the polyploid hybrid seed is induced via complementation of one of the MiMe loci. FIG. 24A illustrates generation of a plant having a first exemplary partially-complemented MiMe genotype where MiMe loci are propagated at the parent stage and each parent has a different set of edited MiMe loci. FIG. 24B illustrates generation of a plant having a second exemplary partially-complemented MiMe genotype where MiMe loci are propagated at the parent stage and each parent has a different set of edited MiMe loci. FIG. 24C illustrates generation of a plant having a third exemplary partially-complemented MiMe genotype where MiMe loci are propagated at the parent stage and each parent has a different set of edited MiMe loci.

FIGS. 25A-25B show the screening results for guide RNA screening for edits in potato. FIG. 25A shows the screening results where editing efficiency is displayed as the proportion edited (vertical axis, in percentage) across a range of selected protospacers (horizontal axis) across four different genes (horizontal axis, grey boxes). From left to right, the potato orthologs are listed for OSD1 (“StOsd1”), PAIR1 (“StPair1”), REC8 (“StRec8”), and SPO11-1 (“StSpo11-1”). FIG. 25B shows further results of screening, as in FIG. 25A, with the exception that the exhibited gene is the potato ortholog of CYCA1 (“StCyca1”).

FIGS. 26A-26F show the resultant editing of MiMe alleles and disruption of MiMe components in edited potato lines. FIG. 26A shows the truncated, inactivated proteins resulting from insertion of a premature stop codon in edited rec8 alleles. FIG. 26B shows the truncated, inactivated proteins resulting from insertion of a premature stop codon in edited cyca1 alleles. FIG. 26C shows the truncated, inactivated proteins resulting from insertion of a premature stop codon in edited spo11-1 alleles. FIG. 26D shows an alignment of edited “scar” nucleotide sequences of edited rec8 alleles (bottom portion continues from the top portion). FIG. 26E shows an alignment of edited “scar” nucleotide sequences of edited cyca1 alleles (bottom portion continues from the top portion). FIG. 26F shows an alignment of edited “scar” nucleotide sequences of edited spo11-1 alleles (bottom portion continues from the top portion).

FIG. 27A shows a summary of the genotyping results at 52 triallelic markers across 19 progeny comprising the Boosted Potato Population 1 (BPP1). The vertical-axis corresponds to individual tetraploid progeny as well as the two diploid parent plants for reference. The horizontal-axis corresponds to individual triallelic markers distributed across 12 separate chromosomes. When referring to the axis, vertical and horizontal are in respect to the legend text for their respective figure. The coloration of each cell denotes the specific configuration of haplotypes observed at that marker (where A=M18, B=DM, and C=M6). Samples with markers that are “AABC” have three haplotypes. The parent genotypes are displayed along the top rows.

FIG. 27B shows a summary of the genotyping results at 60 tetra-allelic markers across 19 progeny comprising the Boosted Potato Population 2 (BPP2). The vertical-axis corresponds to individual tetraploid progeny as well as the two diploid parent plants for reference. The horizontal-axis corresponds to individual tetra-allelic markers distributed across 12 separate chromosomes. When referring to the axis, vertical and horizontal are in respect to the legend text for their respective figure. The coloration of each cell denotes the specific configuration of haplotypes observed at that marker (where A=DM, B=M6, and C=Atlantic Haplotype 1, and D=Atlantic Haplotype 2). Samples with markers that are “ABCD” have four haplotypes. The parent genotypes are displayed along the top rows.

FIG. 27C shows a summary of the genotyping results at 50 triallelic markers across 10 progeny comprising the Boosted Potato Population 3 (BPP3). The vertical-axis corresponds to individual tetraploid progeny as well as the two diploid parent plants for reference. The horizontal-axis corresponds to individual triallelic markers distributed across 12 separate chromosomes. When referring to the axis, vertical and horizontal are in respect to the legend text for their respective figure. The coloration of each cell denotes the specific configuration of haplotypes observed at that marker (where A=M18, B=DM, and C=M6). Samples with markers that are “ABBC” have three haplotypes. The parent genotypes are displayed along the top rows.

FIG. 27D summary of the genotyping results at 60 tetra-allelic markers across 12 progeny comprising the Boosted Potato Population 4 (BPP4). The vertical-axis corresponds to individual tetraploid progeny as well as the two diploid parent plants for reference. The horizontal-axis corresponds to individual tetra-allelic markers distributed across 12 separate chromosomes. When referring to the axis, vertical and horizontal are in respect to the legend text for their respective figure. The coloration of each cell denotes the specific configuration of haplotypes observed at that marker (where A=DM, B=M6, and C=Atlantic Haplotype 1, and D=Atlantic Haplotype 2). Samples with markers that are “ABCD” have four haplotypes. The parent genotypes are displayed along the top rows.

FIG. 27E shows a summary of the genotyping results at 60 tetra-allelic markers across 8 progeny comprising the Boosted Potato Population 5 (BPP5). The vertical-axis corresponds to individual tetraploid progeny as well as the two diploid parent plants for reference. The horizontal-axis corresponds to individual tetra-allelic markers distributed across 12 separate chromosomes. When referring to the axis, vertical and horizontal are in respect to the legend text for their respective figure. The coloration of each cell denotes the specific configuration of haplotypes observed at that marker (where A=DM, B=M6, and C=Atlantic Haplotype 1, and D=Atlantic Haplotype 2). Samples with markers that are “ABCD” have four haplotypes. The parent genotypes are displayed along the top rows.

FIG. 28A shows a photograph of one BPP1 potato plant (“BPP1 (Boosted)”), its grandparent plants (“Grandparent non-MiMe (PED-PR-AA)” and “Grandparent non-MiMe (PED-PR-BB)”), its parent plants (“Parent non-MiMe (PED-PR-CC-o)” and “Parent MiMe (PED-PR-AB-sp)”), and an elite commercial tetraploid at 12 days post-planting. FIG. 28B shows measurements of leaf surface area visible in FIG. 28A, comparing the aerial surface area (cm²) for each individual plant from the photograph in FIG. 28A using ImageJ software.

FIG. 28C shows photographs of three BPP1 potato plants (“BPP1 (Boosted)”), its grandparent plants (“Grandparent non-MiMe (PED-PR-AA)” and “Grandparent non-MiMe (PED-PR-BB)”), its parent plants (“Parent non-MiMe (PED-PR-CC-o)” and “Parent MiMe (PED-PR-AB-sp)”, and an elite commercial tetraploid at 12 days post-planting. FIG. 28D shows measurements of leaf surface area visible in FIG. 28C, comparing the aerial surface area (cm²) for each individual plant from the photograph in FIG. 28C using ImageJ software.

FIG. 28E shows photographs of one BPP1 potato plant (“BPP1 (Boosted)”), its grandparent plants (“Grandparent non-MiMe (PED-PR-AA)” and “Grandparent non-MiMe (PED-PR-BB)”), its parent plants (“Parent non-MiMe (PED-PR-CC-o)” and “Parent MiMe (PED-PR-AB-sp)”), and an elite commercial tetraploid at 19 days post-planting. FIG. 28F shows measurements of leaf surface area visible in FIG. 28E, comparing the aerial surface area (cm²) for each individual plant from the photograph in FIG. 28E using ImageJ software.

FIG. 28G shows photographs of one BPP1 potato plant (“BPP1 (Boosted)”), its grandparent plants (“Grandparent non-MiMe (PED-PR-AA)” and “Grandparent non-MiMe (PED-PR-BB))”), its parent plants (“Parent non-MiMe (PED-PR-CC-o)” and “Parent MiMe (PED-PR-AB-sp)”), and an elite commercial tetraploid at 26 days post-planting. FIG. 28H shows measurements of leaf surface area visible in FIG. 28G, comparing the aerial surface area (cm²) for each individual plant from the photograph in FIG. 28G using ImageJ software.

FIG. 28I shows photographs of six BPP1 potato plants (right, “BPP1”) resulting from the cross between a parent non-MiMe plant (PED-PR-CC-o) and a parent MiMe plant (PED-PR-AB-sp), compared to six SPP2 potato plants (left, “SPP2”) resulting from the cross between two parent non-MiMe plants (PED-PR-CC-o and PED-PR-AB). All the plants are 16 days post-planting. FIG. 28J shows measurements of leaf surface area visible in FIG. 28I, comparing the aerial surface area (cm²) for each individual plant from the photograph in FIG. 28I using ImageJ software. The aerial surface area mean and standard deviation for each plant population are represented in the graph by a cross and error bars, respectively.

FIG. 28K shows photographs of six BPP1 potato plants (right, “BPP1”) resulting from the cross between a parent non-MiMe plant (PED-PR-CC-o) and a parent MiMe plant (PED-PR-AB-sp), compared to six SPP2 potato plants (left, “SPP2”) resulting from the cross between two parent non-MiMe plants (PED-PR-CC-o and PED-PR-AB). All the plants are 23 days post-planting. FIG. 28L shows measurements of leaf surface area visible in FIG. 28K, comparing the aerial surface area (cm²) for each individual plant from the photograph in FIG. 28K using ImageJ software. The aerial surface area mean and standard deviation for each plant population are represented in the graph by a cross and error bars, respectively.

FIG. 29A shows photographs of a BPP2 potato plant (“BPP2 (Boosted)”) adjacent to its grandparent plants (“Grandparent non-MiMe (PED-PR-AA)” and “Grandparent non-MiMe (PED-PR-BB)”), parent plants (“Parent MiMe (PED-PR-AB-sp)” and “Parent MiMe (PED-PR-EF-rso-1)”), and an elite commercial tetraploid. All the plants are 23 days post-planting.

FIG. 29B shows photographs of a BPP2 potato plant (“BPP2 (Boosted)”) adjacent to its grandparent plants (“Grandparent non-MiMe (PED-PR-AA)” and “Grandparent non-MiMe (PED-PR-BB)”, parent plants (“Parent MiMe (PED-PR-AB-sp)” and “Parent MiMe (PED-PR-EF-rso-1”), and an elite commercial tetraploid. All the plants are 23 days post-planting.

FIG. 29C shows photographs of a BPP2 potato plant (“BPP2 (Boosted)”, 4 haplotypes) adjacent to tetraploid (4n) versions of its respective grandparent plants (“4n version of grandparent non-MiMe (PED-PR-AA), 1 haplotype” and “4n version of grandparent non-MiMe (PED-PR-BB), 1 haplotype”) and parent plants (“4n version of PED-PR-AA x PED-PR-BB, 2 haplotypes” and “4n version of Atlantic dihaploid, 2 haplotypes”). All the plants are 23 days post-planting.

FIG. 29D shows photographs of a BPP2 potato plant (“BPP2 (Boosted), 4 haplotypes”) adjacent to tetraploid (4n) versions of its respective grandparent plants (“4n version of grandparent non-MiMe (PED-PR-AA), 1 haplotype” and “4n version of grandparent non-MiMe (PED-PR-BB), 1 haplotype”) and parent plants (“4n version of PED-PR-AA x PED-PR-BB, 2 haplotypes” and “4n version of Atlantic dihaploid, 2 haplotypes”). All the plants are 29 days post-planting.

FIG. 29E shows photographs of nine BPP2 potato plants (right, “BPP2 (PED-PR-EF-rso-1 x PED-PR-AB-sp”) resulting from the cross between a parent MiMe plant (PED-PR-AB-sp) and a parent MiMe plant (PED-PR-EF-rso-1), compared to eight potato plants (left, “SPP1 (PED-PR-EF-o x PED-PR-AABB”) resulting from the cross between two parent non-MiMe plants (PED-PR-EF-o and PED-PR-AABB). One of the progeny plants from the non-MiMe cross failed to develop, which is why there are only eight instead of nine. All the plants are 16 days post-planting. FIG. 29F shows measurements of leaf surface area visible in FIG. 29E, comparing the aerial surface area (cm²) for each individual plant from the photograph in FIG. 29E using ImageJ software. The aerial surface area mean and standard deviation for each plant population are represented in the graph by a cross and error bars, respectively.

FIG. 29G shows photographs of nine BPP2 potato plants (right, “BPP2 (PED-PR-EF-rso-1 x PED-PR-AB-sp”) resulting from the cross between a parent MiMe plant (PED-PR-AB-sp) and a parent MiMe plant (PED-PR-EF-rso-1), compared to eight potato plants (left, “SPP1 (PED-PR-EF-o x PED-PR-AABB”) resulting from the cross between two parent non-MiMe plants (PED-PR-EF-o and PED-PR-AABB). One of the progeny plants from the non-MiMe cross failed to develop, which is why there are only eight instead of nine. All the plants are 23 days post-planting. FIG. 29H shows measurements of leaf surface area visible in FIG. 29G, comparing the aerial surface area (cm²) for each individual plant from the photograph in FIG. 28G using ImageJ software. The aerial surface area mean and standard deviation for each plant population are represented in the graph by a cross and error bars, respectively.

FIG. 29I shows photographs of eight BPP2 potato plants (right, “BPP2 (PED-PR-EF-rso-1 x PED-PR-AB-sp”) resulting from the cross between a parent MiMe plant (PED-PR-AB-sp) and a parent MiMe plant (PED-PR-EF-rso-1), compared to eight potato plants (left, “SPP1 (PED-PR-EF-o x PED-PR-AABB”) resulting from the cross between two parent non-MiMe plants (PED-PR-EF-o and PED-PR-AABB). One of the progeny from the non-MiMe cross failed to develop, which is why there are only eight instead of nine and, because of this, one of the BPP2 was removed so that both populations had the same number of individuals. All the plants are 29 days post-planting.

FIGS. 30A-30G show the superior uniformity of plants grown from populations of polyploid seed produced by the methods described herein as compared to control populations. FIG. 30A shows a matrix for pairwise identity as estimated by the Jaccard similarity coefficient of 64 molecular markers genotyped between 19 tetraploid potato plants in Boosted Potato Population 1 (BPP1). FIG. 30B shows a matrix for pairwise identity as estimated by the Jaccard similarity coefficient of 52 molecular markers genotyped between 19 tetraploid potato plants in Boosted Potato Population 2 (BPP2). FIG. 30C shows a matrix for pairwise identity as estimated by the Jaccard similarity coefficient of 47 molecular markers genotyped between 10 tetraploid potato plants in Boosted Potato Population 3 (BPP3). FIG. 30D shows a matrix illustrating pairwise identity as estimated by the Jaccard similarity coefficient of 60 molecular markers genotyped between 12 tetraploid potato plants in Boosted Potato Population 4 (BPP4). FIG. 30E shows a matrix illustrating pairwise identity as estimated by the Jaccard similarity coefficient of 60 molecular markers genotyped between 8 tetraploid potato plants in Boosted Potato Population 5 (BPP5). FIG. 30F shows a matrix for pairwise identity as estimated by the Jaccard similarity coefficient of 56 molecular markers genotyped between 7 tetraploid potato plants in Standard Potato Population 1 (SPP1). FIG. 30G shows a matrix for pairwise identity as estimated by the Jaccard similarity coefficient of 52 molecular markers genotyped between 4 tetraploid potato plants in Standard Potato Population 2 (SPP2).

FIG. 31A-31B show and measure the comparative tuber sizes of BPP1 and the crosses that generate BPP1. FIG. 31A shows images of the tubers. From left to right, displayed are the tubers of the first grandparent plant (“Grandparent non-MiMe PED-PR-AA (doubled monoploid DM1-3)”) with average tuber weight [35.5 g]; the tubers of the second grandparent plant (“Grandparent non-MiMe PED-PR-BB (selfed M6)”) with average tuber weight [5.2 g]; the tubers of the first parent plant (“Parent MiMe (spo11-1/ps) PED-PR-AB-sp”) with average tuber weight [78.1 g]; the tubers of the second parent plant (“Parent non-MiMe PED-PR-CC-o”) with average tuber weight [35.3 g]; and the tubers of the BPP1 plant (“BOOSTED progeny BPP1”) with average tuber weight [161.2 g]. FIG. 31B shows plotted weights of the tubers displayed in FIG. 31A.

FIG. 32 depicts a plasmid map of pOGZ1, used to introduce MiMe gene edits in Zea mays.

FIGS. 33A-33B show the screening results for guide RNA screening for edits in maize. FIG. 33A shows the screening results where editing efficiency is displayed as the proportion of sequence reads edited compared to wild-type (vertical axis, in percentage) across a range of selected protospacers (horizontal axis) across four different genes (horizontal axis, grey boxes). From left to right, the maize orthologs are listed for OSD1 on chromosome 2 (“ZmOsd1-chr2”), OSD1 on chromosome 5 (“ZmOsd1-chr5-1”) and OSD1 duplication on chromosome 5 (“ZmOsd1-chr5-2”), CYCA1 on chromosome 3 (“ZmTAM-chr3”), and CYCA1 on chromosome 8 (“ZmTAM-chr8”). FIG. 33B shows further results of screening, as in FIG. 33A, with the exception that the exhibited genes are the maize orthologs of PAIR1 (“ZmPair1-1” and “ZmPair1-9”), REC8 (“ZmRec8”), and SPO11-1 (“ZmSpo11-1”).

FIGS. 34A-34F show the resultant editing of MiMe alleles and disruption of MiMe components in edited maize lines. FIG. 34A shows that four conserved amino acids in the REC8 protein are deleted as a result of the edited rec8 allele. FIG. 34B shows the truncated, inactivated OSD1 proteins resulting from frameshift edits that result in premature stop codons in the edited osd1 alleles. FIG. 34C shows the truncated, inactivated SPO11-1 protein resulting from a frameshift edit that results in a premature stop codon in the edited spo11-1 allele. FIG. 34D shows an alignment of edited “scar” nucleotide sequence of the edited rec8 allele (bottom portion continues from the top portion). FIG. 34E shows an alignment of edited “scar” nucleotide sequences of edited osd1 alleles (bottom portion continues from the top portion). FIG. 34F shows an alignment of edited “scar” nucleotide sequences of edited spo11-1 alleles (bottom portion continues from the top portion).

FIG. 35 illustrates the expected pairwise identity (or percentage of shared alleles as estimated by the Jaccard similarity coefficient among genotypes) of randomly selected individuals within each population (SAP—Standard Arabidopsis Population, SPP1—Standard Potato Population 1, SPP2—Standard Potato Population 2, BAP1—Boosted Arabidopsis Population 1, BMP—Boosted Maize Population, BPP1—Boosted Potaro Population 1, BPP2—Boosted Potato Population 2, BPP3—Boosted Potato Population 3, BPP4—Boosted Potaro Population 4, BPP5—Boosted Potato Population 5). The diamonds denote the expected or mean pairwise identity between genotyped plants in each population. The black bars are the median or second quartile. The boxes each represent the interquartile range from the first to the third quartile, and the dashed whiskers display the minimum and maximum pairwise identity between genotyped individuals in each population.

FIG. 36A shows a matrix for pairwise identity as estimated by the Jaccard similarity coefficient of 44 molecular markers genotyped between 48 tetraploid maize plants in Boosted Maize Population (BMP).

FIG. 36B shows a summary of the genotyping results at 44 triallelic markers across 48 progeny comprising the Boosted Maize Population (BMP). The horizontal axis corresponds to individual tetraploid progeny as well as the two diploid parent plants for reference. The vertical axis corresponds to individual triallelic markers distributed across 8 separate chromosomes. The coloration of each cell denotes the specific configuration of haplotypes observed at that marker (where A=LH244, B=A188, and C=FFMM-AT6). Samples with markers that are “AABC” have three haplotypes. Marker design was limited to regions of the genome where heterozygous parental SNPs were present. No usable markers were found on chromosome 2 or chromosome 10. The parent genotypes are displayed along the left-most rows.

FIGS. 37A-37D show examples of BMP individuals. FIG. 37A shows multiple rows of BMP plants. FIG. 37B shows a close-up of the right-hand side of FIG. 37A, displaying a close-up view of three BMP plants. FIG. 37C shows healthy BMP plants readily producing ears. FIG. 37D shows a close-up view of the ears of BMP plants as shown in FIG. 37C.

FIG. 38 shows the results for guide RNA screening for edits in A. thaliana. Editing efficiency is displayed as the proportion of sequence reads edited compared to wild-type (vertical axis, in percentage) across a range of selected protospacers (horizontal axis) across four different genes (horizontal axis, grey boxes). From left to right, the A. thaliana orthologs are listed for OSD1 (“AtOsd1 (Col-0)”), PAIR1 (“AtPair1 (Col-0)”), REC8 (“AtRec8 (Col-0)”), and SPO11-1 (“AtSpo11-1 (Col-0)”). The black arrows indicate which sites were explored further, based on the guide RNA screening results.

FIGS. 39A-39H show the resultant editing of MiMe alleles and disruption of MiMe components in edited Arabidopsis lines. FIG. 39A shows the truncated, inactivated proteins resulting from premature stop codons that result from the edited osd1 alleles. FIG. 39B shows the truncated, inactivated proteins resulting from insertion of a premature stop codon in edited spo11-1 alleles. FIG. 39C shows a truncated, inactivated protein resulting from a premature stop codon in one of the edited rec8 alleles, and the deletion of three conserved amino acids resulting from the other edited rec8 allele. FIG. 39D shows an alignment of edited “scar” nucleotide sequences of edited osd1 alleles targeted at a first target site (bottom portion continues from the top portion). FIG. 39E shows an alignment of edited “scar” nucleotide sequences of edited osd1 alleles targeted at a second target site (bottom portion continues from the top portion). FIG. 39F shows an alignment of edited “scar” nucleotide sequences of edited spo11-1 alleles (bottom portion continues from the top portion).

FIG. 39G shows an alignment of edited “scar” nucleotide sequences of edited rec8 alleles. FIG. 39H shows an alignment of edited “scar” nucleotide sequences of edited pair1 alleles (bottom portion continues from the top portion).

FIGS. 40A-40D show heatmaps comparing genotype distributions and measures of uniformity in MiMe and control populations of A. thaliana. FIG. 40A illustrates a summary of the genotyping results at 58 triallelic markers across 99 progeny comprising the Boosted Arabidopsis Population 1 (BAPI). The horizontal-axis corresponds to individual tetraploid progeny as well as the two diploid parent plants for reference. The vertical-axis corresponds to individual triallelic markers distributed across 4 separate chromosomes. When referring to the axis, vertical and horizontal are in respect to the legend text for their respective figure. The coloration of each cell denotes the specific configuration of haplotypes observed at that marker (where A=Shahdara, B=Col-0, and C=HR-10). Samples with markers that are “AABC” have three haplotypes. Marker design was limited to regions of the genome where heterozygous parental SNPs were present. No usable markers were found on chromosome 3. The parent genotypes are displayed in the two left-most columns. FIG. 40B illustrates a summary of the genotyping results at 57 triallelic markers across 9 progeny of the Standard Arabidopsis Population (SAP, top). The vertical-axis corresponds to individual tetraploid progeny as well as the two tetraploid parent plants for reference. The horizontal-axis corresponds to individual triallelic markers distributed across 5 separate chromosomes. The coloration of each cell denotes the specific configuration of haplotypes observed at that marker (where A=Shahdara, B=Col-0, and C=HR-10). 9 representative individuals from BAPI (FIG. 40A) are shown for comparison (bottom). FIG. 40C shows a matrix illustrating pairwise identity as estimated by the Jaccard similarity coefficient of 57 molecular markers genotyped between 9 tetraploid Arabidopsis plants in the Standard Arabidopsis Population (SAP). FIG. 40D shows a matrix for pairwise identity as estimated by the Jaccard similarity coefficient of 58 molecular markers genotyped between 99 tetraploid Arabidopsis plants in Boosted Arabidopsis Population 1 (BAPI).

FIGS. 41A-41B show BAPI plants. FIG. 41A shows a top-down view of BAPI plants at 48 days after planting. FIG. 41B shows a subset of FIG. 41A's BAPI plants, at a closer proximity and an oblique view, also at 48 days after planting.

FIGS. 42A-42D show formation of parthenocarpic (seedless) fruits across MiMe Arabidopsis plants and controls. FIG. 42A shows seedless fruit from BAPI plants whose pistils were treated, untreated, or mock-treated with gibberellic acid (“GA3”) to induce fruit development, compared to fruit resulting from pistils treated or mock-treated from fertile Arabidopsis control plants. FIG. 42B shows comparison of the average number of seeds per silique (vertical axis) between BAPI plants, the doubled version of the parent MiMe plant PED-AR-BC (doubled as “PED-AR-BCBC”) plants, and the doubled version of the parent MiMe plant PED-AR-AA (doubled as “PED-AR-AAAA”) plants, all along the horizontal axis. The population means and standard deviations are represented in the graph by crosses and error bars, respectively FIG. 42C shows comparison of the silique lengths (vertical axis) between the same plant groups in FIG. 42B (horizontal axis). The length of fruit (siliques) resulting from GA3-treated pistils, mock-treated pistils (“mock”), and untreated pistils was measured across the respective plant groups at 8 days after GA3 application. The population means and standard deviations are represented in the graph by crosses and error bars, respectively. FIG. 42D illustrates a comparison of the average number of seeds per silique (vertical axis) between BAP2 plants, the doubled version of the parent MiMe plant PED-AR-BC (doubled as “PED-AR-BCBC”), and the doubled version of the parent MiMe plant PED-AR-DE (doubled as “PED-AR-DEDE”), all along the horizontal axis. The population means and standard deviations are represented in the graph by crosses and error bars, respectively.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Overview

In one aspect, described herein is a population of polyploid seed comprising three or more haplotypes of the same or related species of plant, wherein at least 50% of the population of polyploid seed is genetically uniform, and wherein the population was obtained from a single plant or a set of plants such as, for example, a set of genetically uniform F1 hybrids. In some embodiments, the population of polyploid seed comprises a subpopulation of genetically uniform polyploid seed in an amount of at least 50% of the total number of seed, the genetically uniform polyploid seed comprising three or more haplotypes of the same or related species of plant. The genetic uniformity of the seeds of the population addresses a long-felt need for genetically uniform populations of polyploid seed comprising three or more haplotypes with improved heterotic performance over the pairs of haplotypes present in existing hybrid plants. In some embodiments, the population of polyploid seed and/or the subpopulation of genetically uniform polyploid seed comprises one or more genetic modifications resulting in decreased expression of one or more MiMe loci. The population of polyploid seed and/or the subpopulation of genetically uniform polyploid seed may have a complete or partial MiMe genotype comprising MiMe alleles conferring decreased expression of MiMe loci of one or more MiMe components. In certain embodiments, germination of a seed of the population of polyploid seed and/or the subpopulation of genetically uniform polyploid seed results in a sterile plant that produces inviable gametes, seedless fruit, or a combination thereof.

In another aspect, provided herein are methods of producing a population of polyploid seed comprising three or more haplotypes wherein at least 50% of the population of polyploid seed are genetically uniform. In some embodiments, the population of polyploid seed comprises a subpopulation of genetically uniform polyploid seed in an amount of at least 50% of the total number of seed, the genetically uniform polyploid seed comprising three or more haplotypes of the same or related species of plant. In some embodiments, the method comprises (a) providing clonal gametes from a pair of parent MiMe plants that together comprise three or more haplotypes; and (b) crossing the clonal gametes to produce the population of polyploid seed. In other embodiments, the method comprises (a) providing clonal gametes from a parent MiMe plant; (b) providing haploid (e.g., monoploid, dihaploid or higher ploidy) gametes from a homozygous parent non-MiMe plant; and (c) crossing the clonal gametes with the haploid (e.g., monoploid) gametes to produce the population of polyploid seed.

In yet another aspect, provided herein are methods of breeding a polyploid hybrid plant line comprising three or more haplotypes, the methods comprising: obtaining a set of lines of a plant; breeding the lines using traditional plant breeding methods to produce a set of candidate lines of the plant; and selecting two or more candidate lines, together comprising three or more haplotypes, for crossing. In some embodiments, after the selection of candidate lines, the methods further comprise generating two parent MiMe plants from the two or more candidate lines; providing clonal gametes from each of the parent MiMe plants; and crossing the clonal gametes to produce a hybrid polyploid seed comprising the three or more haplotypes. In alternative embodiments, after the selection of candidate lines, the methods further comprise generating a single parent MiMe plant from one of the two or more candidate lines; providing clonal gametes from the parent MiMe plant; providing haploid (e.g., monoploid) gametes from a homozygous parent non-MiMe plant of one of the two or more candidate lines; and crossing the clonal gametes with the haploid (e.g., monoploid) gametes to produce a hybrid polyploid seed. In some embodiments, after the crossing of the clonal gametes or the crossing of the clonal gametes with the haploid (e.g., monoploid) gametes, the methods further comprise growing the hybrid polyploid seed to produce a hybrid polyploid plant and evaluating one or more characteristics of the hybrid polyploid plant.

In another aspect, described herein is a population of polyploid seed comprising a partially-complemented MiMe genotype, wherein at least 50% of the population of polyploid seed is genetically uniform, and wherein the population was obtained from a single plant or a set of plants such as, for example, a set of genetically uniform F1 hybrids. In some embodiments, the population of polyploid seed comprises a subpopulation of genetically uniform polyploid seed in an amount of at least 50% of the total number of seed, the genetically uniform seed comprising the partially-complemented MiMe genotype. The polyploid seed comprising the partially-complemented MiMe genotype may comprise one, two, three, or more haplotypes. The partially-complemented MiMe genotype of the population of polyploid seed results in a plant having neither a wild-type meiosis phenotype nor a MiMe phenotype. Thus, in some embodiments, germination of a seed of the population of polyploid seed or the subpopulation of genetically uniform polyploid seed results in a plant that produces inviable gametes. In certain embodiments, the polyploid seed is from a parthenocarpic plant species and results in a plant that produces seedless fruit. The genetic uniformity of the seeds of the population addresses a long-felt need for genetically uniform populations of polyploid seed of plants, including parthenocarpic crops that produce seedless fruit. In some embodiments, the partially-complemented Mime genotype comprises (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second Mime locus of the second MiMe component. In certain embodiments, the partially-complemented MiMe genotype further comprises (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component.

In another aspect, provided herein are methods of producing a population of polyploid seed comprising a partially-complemented MiMe genotype wherein at least 50% of the population of polyploid seed are genetically uniform. In some embodiments, the population of polyploid seed comprises a subpopulation of genetically uniform polyploid seed in an amount of at least 50% of the total number of seed, the genetically uniform seed comprising the partially-complemented MiMe genotype. The polyploid seed comprising the partially-complemented MiMe genotype may comprise one, two, three, or more haplotypes. In some embodiments, the method comprises: (a) providing clonal gametes from a first parent MiMe plant; (b) providing clonal gametes from a second parent MiMe plant; and (c) crossing the clonal gametes to produce the population of polyploid seed comprising a partially-complemented MiMe genotype. In some embodiments, the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component. In certain embodiments, at least one of the MiMe loci having only MiMe alleles of the first MiMe component of the first parent MiMe plant is the same as at least one of the MiMe loci having only MiMe alleles of the first MiMe component of the second parent MiMe plant. In some embodiments, the first and second parent MiMe plants further have only MiMe alleles at one or more MiMe loci of a third MiMe component, wherein the one or more MiMe loci having only MiMe alleles of the third MiMe component of the first and second parent MiMe plants are the same or different.

In yet another aspect, provided herein are methods of breeding a polyploid plant, the methods comprising: obtaining a set of lines of a plant; breeding the lines using traditional plant breeding methods to produce a set of candidate lines of the plant; and selecting two or more candidate lines for crossing. In some embodiments, after the selection of candidate lines, the methods further comprise generating two parent MiMe plants from the two or more candidate lines. In some embodiments, the first parent MiMe plant has only MiMe alleles at one or more MiMe loci of a first MiMe component, only MiMe alleles at a first Mime locus of a second MiMe component, and only non-MiMe alleles at a second MiMe locus of the second MiMe component; and the second parent MiMe plant has only MiMe alleles at one or more Mime loci of the first MiMe component, only non-MiMe alleles at the first MiMe locus of the second MiMe component, and only MiMe alleles at the second MiMe locus of the second MiMe component. In certain embodiments, at least one of the MiMe loci having only MiMe alleles of the first MiMe component of the first parent MiMe plant is the same as at least one of the MiMe loci having only MiMe alleles of the first MiMe component of the second parent MiMe plant. In some embodiments, the first and second parent MiMe plants further have only MiMe alleles at one or more MiMe loci of a third MiMe component, wherein the one or more MiMe loci having only MiMe alleles of the third MiMe component of the first and second parent MiMe plants are the same or different. In some embodiments, the method further comprises providing clonal gametes from each of the parent MiMe plants, crossing the clonal gametes to produce a polyploid seed, growing the polyploid seed to produce a polyploid plant, and evaluating one or more characteristics of the polyploid plant.

In some variations of the methods of breeding, the methods may further comprise repeating the steps of the method, using the one or more characteristics of the hybrid polyploid plant evaluated to guide the breeding of lines, the selecting of candidate lines, or both. In additional variations, the methods may further comprise organizing the set of lines into three or more heterotic groups, wherein each heterotic group comprises a haplotype, and wherein the haplotypes are grouped based on observed or predicted heterotic performance when combined in the hybrid polyploid plant. This method allows for deterministic combination of three or more haplotypes in a polyploid plant, addressing the need for plant breeding methods that yield predictable results on time scales shorter than those required for traditional breeding methods.

### Definitions

As used herein, the term “plant” includes the whole plant or any parts or derivatives thereof, such as plant organs (e.g., harvested or non-harvested flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, tubers, clonally propagated plants, roots, stems, cotyledons, hypocotyls, root tips and the like. The plant parts or derivatives thereof can also include any of the aforementioned plant parts in an encapsulated form such as, for example, shoot meristems, nodes, stolon tips, and the like, encapsulated in alginate, e.g., in a synthetic seed. Any developmental stage is also included, such as seedlings, immature and mature, etc.

As used herein, “potato” typically refers to the species Solanum tuberosum. Moreover, it will be readily apparent to those of ordinary skill in the art that some varieties of Solanum tuberosum include genetic introgressions from related Solanum species, but that such varieties are still considered Solanum tuberosum unless otherwise noted. The terms “potato” and “potato plant” include the whole potato plant or any parts or derivatives thereof, such as plant organs (e.g., harvested or non-harvested flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, tubers, clonally propagated plants, roots, stems, cotyledons, hypocotyls, root tips, meristems, nodes, stolon tips and the like. The potato plant parts or derivatives thereof can also include any of the aforementioned plant parts in an encapsulated form such as, for example, shoot meristems, nodes, stolen tips, and the like, encapsulated in alginate, e.g. in a synthetic seed. Any developmental stage is also included, such as seedlings, immature and mature, etc.

As used herein, the terms “maize” and “maize plant” include the whole maize plant or any parts or derivatives thereof, such as plant organs (e.g., harvested or non-harvested flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, clonally propagated plants, roots, stems, cotyledons, hypocotyls, root tips and the like. The plant parts or derivatives thereof can also include any of the aforementioned plant parts in an encapsulated form such as, for example, shoot meristems, nodes, stolon tips, and the like, encapsulated in alginate, e.g., in a synthetic seed. Any developmental stage is also included, such as seeds, seedlings, immature and mature, etc. As used herein, the term “non-regenerable” generally refers to a maize plant part, a plant cell, a processed maize product, or a portion of any of the foregoing, that cannot be induced to form a whole maize plant or that cannot be induced to form a whole maize plant that is capable of sexual and/or asexual reproduction.

As used herein, the term “seed” typically refers to a true seed rather than another plant part used for propagation. For example, the term “potato seed” as used herein refers to true potato seed rather than a potato tuber.

As used herein, “parthenocarpic” typically describes a plant or crop in which fruit development may proceed independently of pollination, fertilization, and/or seed development. Parthenocarpic plants may produce seedless fruit in a process commonly known as parthenocarpy, which is known in the art and described herein.

As used herein, “seedless plant” typically describes a plant or crop in which fruit produced contain inviable and/or poorly-developed seed. As used herein, seedless plants may or may not require pollen or fertilization to stimulate the development of the fruit. Fruit development may or may not involve initialization and abortion of a seed, leaving an incompletely developed seed. This process is commonly known to occur in plants such as, by way of example only, seedless banana, seedless table grapes and seedless watermelon. As used herein, “seedless” may refer to incomplete development of seed, lack of seed production, or otherwise inviable seed. A seedless plant may also refer to a plant that fails to produce fruits or seeds at all. For example, in a crop where seed development is required for fruit development failed formation of viable eggs, sperm or failed fertilization may result in no seed and no fruit of any kind.

As used herein, the term “non-regenerable” generally refers to a plant part, a plant cell, a processed plant product, or a portion of any of the foregoing, that cannot be induced to form a whole plant or that cannot be induced to form a whole plant that is capable of sexual and/or asexual reproduction.

As used herein, “ploidy” refers to the number of complete sets of chromosomes in a cell or organism. Ploidy may be annotated using “n” as the unit of complete sets of chromosomes. For example, a cell or organism with a single set of chromosomes may be referred to as “in”, or the single set of chromosomes itself may be referred to as “In”. A diploid cell or organism with two sets of chromosomes may be referred to as “2n”; a triploid cell or organism with three sets of chromosomes may be referred to as “3n”; and so on.

As used herein, “monoploid” refers to a cell or organism with a ploidy of 1n.

As used herein, “diploid” refers to a cell or organism with a ploidy of 2n.

As used herein, “polyploid” refers to a cell or organism with a ploidy of greater than 2n. “Polyploid” may refer to organisms which are triploid (3n), tetraploid (4n), pentaploid (5n), hexaploid (6n), heptaploid (7n), octoploid (8n), or higher ploidies (greater than 8n).

As used herein, “allele” refers to one of two or more alternative forms of a single gene or locus within the genome. As used herein, “monoallelic” typically describes the presence of a single allele at a given locus or set of loci within a cell or organism. As used herein, “biallelic” typically describes the presence of two different alleles at a given locus or set of loci within a cell or organism. As used herein, “multiallelic” typically describes the presence of three or more alleles at a given locus or set of loci within a cell or organism.

As used herein, “haplotype” refers to a distinct 1n set of chromosomes with a unique set of alleles. As used herein, each haplotype is distinct from other haplotypes in that it contains a set of alleles that confers a unique set of characteristics not conferred by other haplotypes. As used herein, as a feature of the present disclosure, each distinct haplotype need not be inherited from a different parent—a polyploid organism of the present disclosure may comprise three or more haplotypes inherited from two parents. As used herein, “monoallelic plant” typically refers to a plant line containing a single haplotype, “biallelic plant” typically refers to a plant line containing two haplotypes, and “multiallelic plant” typically refers to a plant line containing three or more haplotypes. In the case of allopolyploid plants that contain multiple subgenomes between which there is little to no recombination, as used herein, the term “three or more haplotypes” typically refers to three or more haplotypes of the same subgenome.

As used herein, “clonal” describes a body of DNA that is substantially identical to another body of DNA; or a set of cells or organisms that comprise such DNA. For example, mitosis results in two clonal genomes comprised by two clonal cells. Due to random errors in natural DNA replication, clonal bodies of DNA, clonal cells, or clonal organisms may not be completely identical. “Clonal” may describe two genomes that are not completely identical in sequence but that contain the same set of alleles.

As used herein, “genetically uniform” typically describes a set of individual plants, plant parts (e.g., seeds), or plant cells whose genomes are identical across at least 80% of loci, or are clonal. Genetic uniformity of a set of individual plants, plant parts (e.g., seeds), or plant cells may be measured using methods known in the art and described herein. For example, a set of genetic markers may be identified and used to determine the estimated pairwise identity of a pair of individuals, or to determine the average pairwise genetic uniformity of a population of individuals, using the Jaccard similarity coefficient. For example, a population of genetically uniform plants or seeds may consist of plants or seeds having genomes that are identical to one another across at least 80%, at least 85%, at least 90%, at least 95%, at least 96%, at least 97%, at least 98%, at least 99%, or 100% of genetic markers analyzed, or may consist of seeds having an average pairwise genetic uniformity of at least 80%, at least 85%, at least 90%, at least 95%, at least 96%, at least 97%, at least 98%, at least 99%, or 100% as measured by the Jaccard similarity coefficient. Additionally, each pair of seeds in a population of genetically uniform plants or seed may have genomes that have a pairwise identity of at least 95%, at least 96%, at least 97%, at least 98%, at least 99%, or 100% as measured by the Jaccard similarity coefficient.

As used herein, “expression” and “expression level” refer to the relative or absolute amount of a functional gene product present in a cell. As used herein, “gene products” include, but are not limited to, nucleic acids (e.g., RNA), post-transcriptionally modified nucleic acids (e.g., spliced RNA, poly-adenylated mRNA), proteins (e.g., enzymes, structural proteins, etc.), and post-translationally modified proteins (e.g., glycoproteins, lipoproteins, etc.). The function of the gene product refers to the wild-type, unmodified, uninhibited function of the gene product. As used herein, “decreased expression” refers to a relative decrease in the amount of a functional gene product of a gene or genetic locus, such as a MiMe locus, present in a cell. The decreased expression may refer to a decrease in the total amount of a gene product present in a cell (e.g., a decrease in the amount of a protein) or to a decrease in the amount of functional gene products present in a cell (e.g., a decrease in the percentage of proteins with wild-type function, e.g., an altered activity of the protein) or to a decrease in the function of gene products present in a cell (e.g., a decrease in the activity of proteins as compared to proteins with wild-type function, e.g., elimination of activity). The decreased expression may be of a gene product encoded at a certain genomic locus. Decreased expression also includes “non-expression” and “eliminated expression.” As used herein, “non-expression” or “eliminated expression” refers to the absence of a functional gene product present in a cell, or to an expression level insufficient for detection of the gene product in the cell, or to an expression level insufficient to result in the function of the gene product within the cell, or to an activity level insufficient to result in the detectable activity of the gene product within the cell.

As used herein, “homozygous” describes a cell or organism in which all sets of chromosomes encode the same allele or set of alleles at a certain chromosomal locus, a set of chromosomal loci, or at all chromosomal loci. For example, a triploid cell or organism with the same allele at a specific locus in all three sets of chromosomes is homozygous for that allele. An organism may be homozygous for a specific allele or set of alleles at a certain chromosomal locus or set of chromosomal loci, or an organism may be homozygous for a haplotype. For example, a triploid cell or organism containing three copies of the same haplotype is homozygous for that haplotype. As used herein, “homozygous plant” typically refers to an inbred plant or plant line, a monoallelic plant or plant line, or a plant or plant line which is homozygous at all loci within its genome.

As used herein, “heterozygous” describes a cell or organism in which at least one set of chromosomes encodes an allele or set of alleles at a certain chromosomal locus or set of chromosomal loci that is distinct from those of the other sets of chromosomes within the cell or organism. For example, a triploid cell or organism having allele a₁ at locus A in two sets of chromosomes and having allele a₂ at locus A in the third set of chromosomes is heterozygous for alleles a₁ and a₂. An organism may be heterozygous for a specific set of alleles at a certain chromosomal locus or set of chromosomal loci, or an organism may be heterozygous for a haplotype. For example, a triploid cell or organism containing two copies of one first haplotype and one copy of a second haplotype is heterozygous for the first and second haplotype. As used herein, “heterozygous plant” typically refers to a biallelic or multiallelic plant or plant line containing two or more haplotypes.

As used herein, “crossing” refers to the act of forming a zygote from gametes of two distinct plants or plant lines. Crossing may refer to pollinating a plant or plant line using the pollen of a different plant or plant line.

As used herein, “related species of plant” refers to two or more species that, when crossed, result in viable seed.

As used herein, “hybrid” describes a plant comprising two or more haplotypes from the same or related species of plant.

As used herein, “F1 hybrid” refers to the first filial generation of hybrid seeds or plants resulting from the cross of parents comprising two or more haplotypes. For clarity, this refers to the first filial generation of the cross and not the first filial generation of the hybrids of a cross.

As used herein, “heterotic performance” refers to the performance of a set of two or more haplotypes in conferring certain desirable characteristics when combined in a hybrid plant. The desired characteristics of heterotic performance may include characteristics of plant vigor including, but not limited to, plant size, hardiness, fruit or grain yield, and the like.

As used herein, “MiMe” typically refers to a phenotype of a plant wherein the wild-type meiosis phenotype of the plant is disrupted in such a way that results in the formation of clonal female gametes and/or clonal male gametes. “MiMe” may refer to any one of several known methods to promote the formation of clonal female gametes and/or clonal male gametes in plants including, but not limited to, Mitosis instead of Meiosis as disclosed in d'Erfurth et al. (2009. Turning meiosis into mitosis. PLoS Biol 7, e1000124) and first division restitution without crossing over (FDR-NCO) as disclosed in Peloquin et al. (1999. Meiotic mutants in potato: valuable variants. Genetics 153: 1493-1499), a prime example of FDR-NCO being spo11-1, psi mutants as disclosed in Brownfield and Kohler (2010. Unreduced gamete formation in plants: mechanisms and prospects. J Exp Bot 62:5, 1659-1668).

In organisms with a wild-type meiosis phenotype, meiosis in germline cells results in haploid gametes. As used herein, “haploid” typically refers to a cell or organism with a ploidy half that of the parent organism. As used herein, “haploid gametes” typically refers to gamete cells with a ploidy half that of the parent organism. For example, in a diploid (2n) organism with a wild-type meiosis phenotype, meiosis in germline cells results in 1n haploid gametes. In another example, in a tetraploid (4n) organism with a wild-type meiosis phenotype, meiosis in germline cells results in 2n haploid gametes. As used herein, “parent non-MiMe plant” typically refers to a plant with the wild-type meiosis phenotype wherein meiosis in germline cells results in haploid gametes (pollen and egg cells). As used herein, “homozygous parent non-MiMe plant” typically refers to an inbred parent non-MiMe parent, a monoallelic parent non-MiMe plant, or a parent non-MiMe plant which is homozygous at all loci within its genome. A homozygous parent non-MiMe plant may be produced through inbreeding, production of a doubled haploid line (e.g., a doubled monoploid line), or any other method known in the art for creating plant lines with high degrees of homozygosity.

In plants with the MiMe phenotype, meiosis is replaced by a mitosis-like process in male and/or female germline cells, resulting in clonal gametes. As used herein, “clonal gametes” typically refers to gametes which comprise unreduced, unrecombined copies of the parent plant's genome and, therefore, have the same ploidy as, and are typically genetically identical to, the parent plant. Clonal gametes are produced when germline cells in the parent plant do not undergo recombination as they would in a normal meiotic process, and also undergo a first division restitution or a second division restitution, resulting in unreduced gametes. As a result, clonal gametes are typically both unreduced and unrecombined and therefore typically genetically identical to the parent plant. For example, in a diploid (2n) plant with a MiMe phenotype, germline cells undergo mitosis instead of meiosis, typically resulting in 2n unrecombined gametes, i.e., clonal gametes. In another example, in a tetraploid (4n) plant with a MiMe phenotype, germline cells undergo mitosis instead of meiosis, typically resulting in 4n unrecombined gametes, i.e., clonal gametes. Clonal gametes may refer to female clonal gametes, male clonal gametes, or a combination thereof.

As used herein, “unreduced, non-clonal gametes” typically refers to gametes which comprise unreduced, yet recombined, copies of the parent plant's genome and, therefore, have the same ploidy as the parent plant, but are not genetically identical to the parent plant. Unreduced, non-clonal gametes are produced when germline cells in the parent plant undergo recombination as they would in a normal meiotic process, but undergo a first division restitution or a second division restitution, resulting in unreduced gametes. Therefore, even though unreduced, non-clonal gametes are unreduced, they are the result of a normal recombination process, and are therefore not genetically identical to the parent plant. For example, germline cells in a diploid (2n) plant that undergo a normal recombination process but undergo a first division restitution or a second division restitution result in 2n, recombined gametes, i.e., unreduced, non-clonal gametes. In another example, germline cells in a tetraploid (4n) plant that undergo a normal recombination process but undergo a first division restitution or a second division restitution result in 4n, recombined gametes, i.e., unreduced, non-clonal gametes. Unreduced, non-clonal gametes may refer to female clonal gametes, male clonal gametes, or a combination thereof.

As used herein, “MiMe component” typically refers to a gene function that contributes to a MiMe phenotype, including, but not limited to, genes and gene products involved in meiosis that may be modified or altered to disrupt a wild-type meiotic phenotype in a manner relevant to the formation of clonal female gametes and/or clonal male gametes via MiMe. MiMe components include (1) a component of sister chromatid cohesion during the first division of meiosis, (2) a component of DNA double strand breakage during meiotic recombination, (3) a component of progression through the second division of meiosis, and (4) a component of progression through the first division of meiosis. In general, each MiMe component includes one or more MiMe loci discussed further below. Further, generally, each MiMe locus may have MiMe alleles and non-MiMe alleles.

As used herein, “MiMe allele” typically refers to an allele at a MiMe locus that disrupts the normal meiotic function of a MiMe component (e.g., an allele that disrupts sister chromatid cohesion during the first division of meiosis). MiMe alleles may be naturally occurring MiMe alleles, or may be introduced into a plant line via genetic modification using the methods described herein.

As used herein, “non-MiMe allele” typically refers to any allele that contributes to wild-type function of a MiMe component that therefore does not contribute to conferring a MiMe phenotype in a plant. A non-MiMe allele typically refers to an allele at a MiMe locus that contributes to the wild-type meiotic function of a MiMe component (e.g., an allele that provides the wild-type function that contributes to sister chromatid cohesion during the first division of meiosis).

As used herein, “complete MiMe genotype” typically refers to any set of alleles that confers the MiMe phenotype in a plant. The individual alleles that make up the complete MiMe genotypes are referred to as MiMe alleles. The complete MiMe genotype may be naturally present in a plant or may be introduced via, for example, plant breeding, transgenic techniques, gene-editing techniques, or any combination thereof to introduce one or more naturally-occurring alleles, non-naturally-occurring alleles, or a combination thereof. A complete MiMe genotype may comprise any number of MiMe alleles that results in a MiMe phenotype, such as one, two, three, or more MiMe alleles. As used herein, “MiMe locus” and “MiMe loci” typically refers to any chromosomal locus or loci which may be the site of MiMe alleles, including genes and intergenic loci. A MiMe locus or MiMe loci may correspond to a specific MiMe component, for example, if the MiMe locus encodes a MiMe component gene product. A complete MiMe genotype may comprise MiMe alleles at any number of MiMe loci, such as one, two, three, or more MiMe loci. A complete MiMe genotype may comprise different alleles at the same MiMe locus on different sets of chromosomes and need not be homozygous to confer a MiMe phenotype. For example, a diploid plant with a complete MiMe genotype may have two different REC8 alleles each of which reduces or eliminates REC8 expression or activity such that the plant has two MiMe alleles for REC8 and thus exhibits disruption of sister chromatid cohesion during the first division of meiosis. Specific examples of complete MiMe genotypes are described in detail herein.

As used herein, “partial MiMe genotype” typically refers to a set of alleles that comprises both MiMe and non-MiMe alleles at one or more MiMe loci such that a plant with a partial MiMe genotype exhibits a wild-type meiosis phenotype. Two plants having compatible partial MiMe genotypes each exhibit a wild-type meiosis phenotype and may be crossed to produce F1 offspring having a complete MiMe genotype and, thus, a MiMe phenotype. As used herein, “compatible partial MiMe genotypes” typically refers to two or more partial MiMe genotypes that comprise sets of MiMe alleles at the same MiMe loci. For example, a partial MiMe genotype comprising MiMe alleles of REC8, SPO11-1, and OSD1 is compatible with another partial MiMe genotype that comprises the same or different MiMe alleles of REC8, SPO11-1, and OSD1. The MiMe alleles of a partial MiMe genotype may be combined with the MiMe alleles of the same or different partial MiMe genotype in a single cross to create a complete MiMe genotype and confer a MiMe phenotype in the F1 offspring. In general, where MiMe alleles and non-MiMe alleles are referred to together they are alleles of the same MiMe loci. The partial MiMe genotype may be naturally present in a plant or may be introduced via, for example, plant breeding, transgenic techniques, gene-editing techniques, or any combination thereof to introduce one or more naturally-occurring alleles, non-naturally-occurring alleles, or a combination thereof. A partial MiMe genotype may comprise any number of alleles, such as one, two, three, or more alleles. Further, a partial MiMe genotype may comprise MiMe alleles at any number of MiMe loci, such as one, two, three, or more MiMe loci. Specific examples of partial MiMe genotypes are described in detail herein.

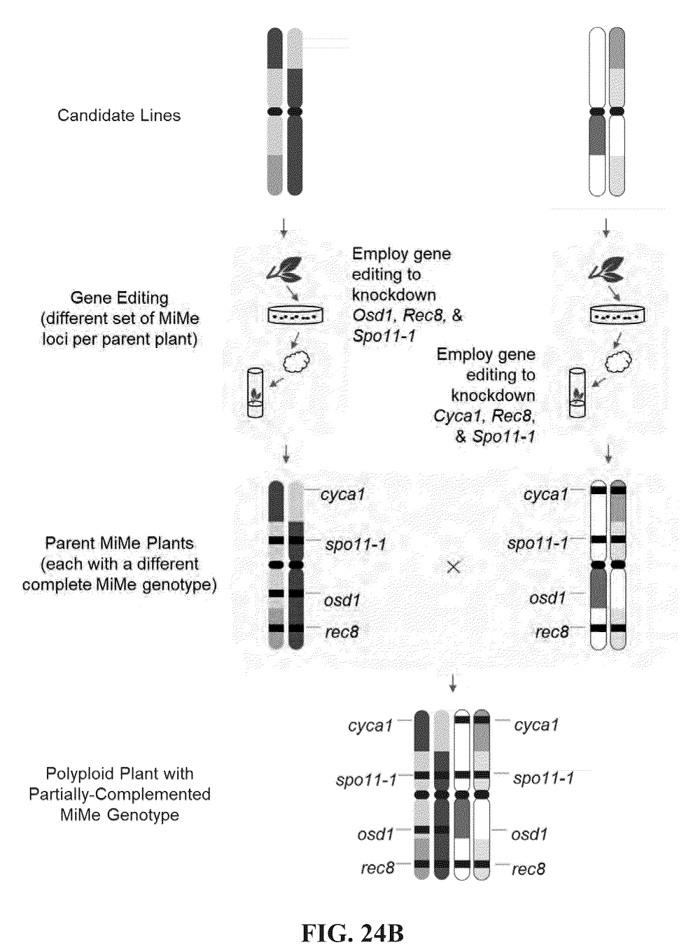

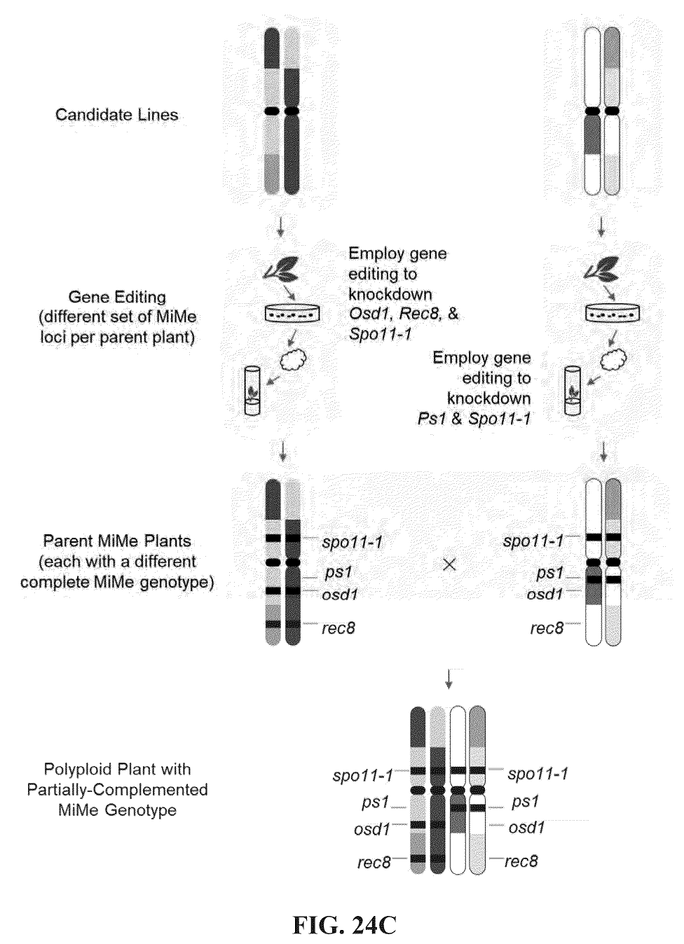

As used herein, “partially-complemented MiMe genotype” typically refers to a set of alleles that comprises only MiMe alleles at each of one or more MiMe loci of a first MiMe component, both MiMe and non-MiMe alleles at a first MiMe locus of a second MiMe component, and both MiMe and non-MiMe alleles at a second MiMe locus of the second MiMe component. A plant with a partially-complemented MiMe genotype typically does not exhibit a wild-type meiosis phenotype because the first MiMe component has only MiMe alleles at each of one or more MiMe loci, disrupting wild-type meiosis. The plant will also not exhibit a MiMe phenotype due to the complementation of the MiMe alleles by the non-MiMe alleles of each of the first and second MiMe loci of the second MiMe component. Therefore, a plant with a partially-complemented MiMe genotype exhibits neither a wild-type meiosis phenotype nor a MiMe phenotype. A partially-complemented MiMe genotype may also include MiMe alleles for a third MiMe component, e.g., only MiMe alleles at each of one or more MiMe loci of a third MiMe component, or both MiMe and non-MiMe alleles at a first MiMe locus of a third MiMe component and both MiMe and non-MiMe alleles at a second MiMe locus of the third MiMe component. As used herein, a plant or genotype “comprising only MiMe alleles at a MiMe locus” is a plant or genotype wherein each set of chromosomes has a MiMe allele at said locus, thus conferring decreased expression (including non-expression or altered activity) of the functional gene product of said locus. Exemplary partially-complemented MiMe genotypes are shown in FIGS. 24A-24C and described in detail herein. The MiMe alleles of each MiMe locus having only MiMe alleles of the first MiMe component may comprise any number of distinct MiMe alleles on different sets of chromosomes and need not be homozygous, as long as there are no non-MiMe alleles at that locus on any set of chromosomes. The partially-complemented MiMe genotype may comprise, for example, one, two, three, or more MiMe alleles and one, two, three, or more non-MiMe alleles at each of the first and second MiMe loci of the second MiMe component, as long as at least one MiMe allele and at least one non-MiMe allele are present at each of the first and second MiMe loci of the second MiMe component. Further, a partially-complemented MiMe genotype may comprise MiMe alleles at more than three MiMe loci, such as four, five, or more MiMe loci. Specific examples of partially-complemented MiMe genotypes are described in detail herein.

As used herein, “parent MiMe plant” typically refers to a plant which has a complete MiMe genotype and exhibits a MiMe phenotype, and which may be a source of clonal gametes (pollen and/or egg cells).

As used herein, “introducing a complete MiMe genotype directly” typically refers to introducing genetic modifications resulting in a complete MiMe genotype into a plant or plant cell including using the methods described herein, selecting a plant or plant cell that has a complete MiMe genotype, if needed, and regenerating the cell that has the complete MiMe genotype into a plant that exhibits a MiMe phenotype, if needed.

As used herein, “grandparent non-MiMe plant having a partial MiMe genotype” typically refers to a plant which has a partial MiMe genotype and exhibits a wild-type meiosis phenotype. A grandparent non-MiMe plant having a partial MiMe genotype produces haploid gametes that may be crossed with haploid gametes from the same or another grandparent non-MiMe plant having a partial MiMe genotype to produce one or more seeds that have a complete MiMe genotype and can be grown to produce one or more parent MiMe plants.

As used herein, “introducing a partial MiMe genotype” refers to introducing genetic modifications resulting in a partial MiMe genotype into a plant or plant cell including using the methods described herein, selecting a plant or plant cell that has a partial MiMe genotype, if needed, and regenerating the cell that has a partial MiMe genotype into a plant that exhibits a wild-type meiosis phenotype. For example, introducing a partial MiMe genotype could include crossing a plant with a MiMe and a non-MiMe allele for a component of sister chromatid cohesion during the first division of meiosis and a MiMe and a non-MiMe allele for a component of DNA double strand breakage during meiotic recombination with a plant that has a MiMe and a non-MiMe allele for a component of progression through the second division of meiosis, and then selecting for offspring that are heterozygous for all three of the parental MiMe alleles and therefore have a partial MiMe genotype.

As used herein, “genetic modification” typically refers to any sequence or portion thereof within a nucleic acid molecule that differs from the sequence of an ancestral nucleic acid molecule. For example, a seed that contains an inserted or deleted genomic sequence that is not present in one of its parent plants comprises a genetic modification. A genetic modification may be naturally occurring or introduced. A genetic modification may be introduced via, for example: plant breeding to introduce a naturally-occurring genetic modification of one plant line into another plant line; transgenic methods; gene editing; chemical mutagenesis; and the like.

As used herein, “transgenesis” refers to the insertion of an exogenous genetic element into the genome of an organism. Any exogenous genetic element may be inserted via transgenesis, including, but not limited to, genes, protein coding sequences, non-protein coding sequences, regulatory sequences, spacer DNA, and the like.

As used herein, “gene editing” refers to a type of genetic modification in which DNA is inserted, deleted or substituted in the genome of an organism using one or more natural or engineered nucleases. Gene editing may be carried out using site-specific nucleases, guided nucleases, or a combination thereof. The nuclease creates one or more site-specific breaks, such as double-strand breaks (DSBs) at target loci in the genome. Each site-specific break may be repaired, for example via non-homologous end joining (NHEJ), resulting in a genetic modification in the genome at the target locus; or via homologous recombination of the target locus with a provided repair nucleic acid molecule comprising homology to the target genomic sequence and the desired genetic modification.

### Populations of Polyploid Seed

In one aspect, described herein is a population of polyploid seed comprising three or more haplotypes of the same or related species of plant, wherein at least 50% of the population of polyploid seed are genetically uniform, and wherein the population was obtained from a single plant or a set of plants (e.g., a set of genetically uniform plants, e.g., a set of genetically uniform F1 hybrids). In some embodiments, the population of polyploid seed comprises a subpopulation of genetically uniform polyploid seed in an amount of at least 50% of the total number of seed, the genetically uniform seed comprising three or more haplotypes of the same or related species of plant. In some embodiments, the population of polyploid seed may have a complete MiMe genotype or a partial MiMe genotype.

In another aspect, described herein is a population of polyploid seed comprising a partially-complemented MiMe genotype, wherein at least 50% of the population of polyploid seed are genetically uniform, and wherein the population was obtained from a single plant or a set of plants (e.g., a set of genetically uniform plants, e.g., a set of genetically uniform F1 hybrids). In some embodiments, the population of polyploid seed comprises a subpopulation of genetically uniform polyploid seed in an amount of at least 50% of the total number of seed, the genetically uniform seed comprising the partially-complemented MiMe genotype. In some embodiments, the population of polyploid seed comprising the partially-complemented MiMe genotype may comprise one, two, three, or more haplotypes.

### Haplotypes

In some embodiments, the population of polyploid seed or the subpopulation of genetically uniform polyploid seed comprises one, two, three, or more haplotypes. In some variations, the population of polyploid seed or the subpopulation of genetically uniform polyploid seed comprises two or more haplotypes, three or more haplotypes, four or more haplotypes, five or more haplotypes, six or more haplotypes, seven or more haplotypes, or eight or more haplotypes. In additional variations, the population of polyploid seed or the subpopulation of genetically uniform polyploid seed comprises two, three, four, five, six, seven, or eight haplotypes. In some embodiments wherein the polyploid seed is from an allopolyploid plant having multiple subgenomes, the two, three, or more haplotypes are two, three, or more haplotypes of the same subgenome.

### Species of Plant

The population of polyploid seed can be of any kind of plant. In some embodiments, the population of polyploid seed is seed of a monocot plant. In other embodiments, the population of polyploid seed is seed of a dicot plant. In some embodiments, the population of polyploid seed is seed of a crop plant. In some variations, the population of polyploid seed can be of any crop plant including, but not limited to, potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, bilberry, lingonberry, cowberry, huckleberry, dewberry, caneberry, loganberry, marionberry, tayberry, plantain, red banana, latundan banana, Cavendish banana, artichoke, beets, potato, sweet potato, achira, ahipa, arracacha, maca, nashua, mauka, oca, ulluco, yacon, yams, radish, horseradish, turnip, parsnip, rutabaga, Yucca, onion, shallot, leek, scallion, garlic, chives, peanut, asparagus, sugarcane, cassava, Brussels sprouts, cabbage, collards, kale, lettuce, chard, spinach, bok choy, okra, cashew nuts, pineapple, celery, birch, rapeseed, mustard, tea, hemp, safflower seed, cedar, Quinoa, chickpea, citron, satsuma, tangerine and mandarin, clementine, coffee, cola, hazelnut, saffron, melon and cantaloupe, carrot, oil palms, teff, rubber rabbit brush, Eucalyptus, fir, soybean, sunflower, hemlock tree, rubber tree, kenaf, hop, walnut, larch, lentil, flax, maple, Miscanthus, basil, olive, millet, pennycress, avocado, green bean, bean, ground cherry, pine, pistachio nut, pea, turf grass, poplar, apricot, plum and prune, almond, nectarine, peach, cherry, rose, Rubus, sesame, sorghum, spruce, switchgrass, Russian dandelion, cacao, durum wheat, spelt, broad bean, cowpea, ginger, kohlrabi, broccoli, cauliflower, wheat, rice, barley, oat, rye, bamboo, ryegrass, lawn grass, or ornamental grass. In additional variations, the population of polyploid seed can be any species of crop plant including, but not limited to, Solanum spp., Solanum chacoense, Solanum tuberosum, Solanum lycopersicum, Solanum melongena, Zea spp. including Z. diploperennis, Z. luxurians, Z. nicaraguensis, and Z. perennis, Zea mays including Z. mays spp. mays (modern maize), Z. mays spp. Parviglumis, and Z. mays spp. mexicana, Musa spp., Musa acuminata, Musa balbisiana, Vaccinium spp., Vaccinium darrowii, Vaccinium corymbosum, Vaccinium erythrocarpum, Vaccinium macrocarpon, Vaccinium microcarpum, Vaccinium oxycoccos Rubus spp., Rubus fruticosus, Rubus idaeus, Rubus fruticosus, Rubus aboriginum, Rubus x loganobaccus, Rubus idaeus and Rubus strigosus Rubus occidentalis, Citrullus spp., Citrullus lanatus, Cucumis spp., Cucumis melo, Physalis spp., Physalis philadelphica, Physalis ixocarpa, Capsicum spp., Capsicum annuum, Capsicum baccatum, Capsicum chinense, Capsicum frutescens, Capsicum pubescens, Vitis spp., Vitis vinifera, Vitis amurensis, Vitis labrusca, Vitis riparia, Vitis rotundifolia, Citrus spp., Citrus sinensis, Citrus reticulata, Citrus aurantium, Citrus bergamia, Citrus limon, Citrus medica, Citrus maxima, Citrus hystrix, Citrus aurantiifolia, Citrus x sinensis, Citrus x paradisi, Cucumis spp., Cucumis sativus, Cucurbita spp., Cucurbita argyrosperma, Cucurbita ficifolia, Cucurbita maxima, Cucurbita moschata, C. pepo, Lagenaria spp., Malus spp., Malus domestica, Pyrus spp., Pyrus communis, Pyrus pyrifolia, Pyrus sinkiangensis, Pyrus pashia, Actinidia spp., Actinidia deliciosa, Punica spp., Punica granatum, Mangifera spp., Mangifera indica, Mangifera foetida, Psidium spp., Psidium guajava, Carica spp., Carica papaya, Persea spp., Persea americana, Prunus spp., Phoenix spp., Phoenix dactylifera, Ficus spp., Ficus carica, Medicago spp., Medicago sativa, Nicotiana spp., Nicotiana tabacum, Nicotiana rustica, Gossypium spp., Gossypium hirsutum, Gossypium barbadense, Gossypium arboretum, Gossypium herbaceum, Trifolium spp., Trifolium repens, Trifolium pretense, Fragaria spp., Fragaria virginiana Fragaria chiloensis, Fragaria vesca, Fragaria x ananassa, Ribes spp., Cynara spp., Cynara cardunculus, Beta spp., Beta vulgaris, Beta vulgaris subsp. vulgaris, Ipomoea spp., Ipomoea batatas, Canna spp., Canna indica, Pachyrhizus spp., Pachyrhizus ahipa, Arracacia spp., Arracacia xanthorrhiza, Lepidium spp., Lepidium meyenii, Mirabilis spp., Mirabilis expansa, Oxalis spp., Oxalis tuberosa, Ullucus spp., Ullucus tuberosus, Smallanthus spp., Smallanthus sonchifolius, Dioscorea spp., Dioscorea rotundata, Dioscorea cayennensis, Dioscorea alata, Dioscorea polystachya, Dioscorea bulbifera, Dioscorea esculenta, Dioscorea dumetorum, Dioscorea trifida, Raphanus spp. Raphanus raphanistrum, Armoracia spp., Armoracia rusticana, Brassica spp., Brassica rapa, Brassica rapa subsp. chinensis, Brassica napus, Brassica oleracea, Brassica oleracea, Brassica oleracea var. oleracea, Brassica oleracea var. italica, Pastinaca spp., Pastinaca sativa, Yucca spp., Allium spp., Allium cepa, Allium ampeloprasum, Allium chinense, Allium fistulosum, Allium x proliferum, Allium sativum, Allium schoenoprasum, Arachis spp., Arachis hypogaea, Asparagus spp., Asparagus officinalis, Saccharum spp., Saccharum officinarum, Saccharum barberi, Saccharum robustum, Saccharum spontaneum, Manihot spp., Manihot esculenta, Lactuca spp., Lactuca sativa, Spinacia spp., and Spinacia oleracea.

In some embodiments, the population of polyploid seed is of an Arabidopsis plant. In certain embodiments, the population of polyploid seed is of an Arabidopsis thaliana plant. In some embodiments, the population of polyploid seed is of a cross of two Arabidopsis thaliana plants derived from two or more lines of Arabidopsis thaliana. In certain embodiments, the two or more lines of Arabidopsis thaliana comprise Col-0, Landsberg, Shahdara, HR-10, Ws-2, Col-0 (CS851557-S1), CS851294, Col X Shahdara F1, Landsberg x Shahdara F1, Col x Shahdara F2, Landsberg x Shahdara F2, Col-0 (CS851557-S1) x HR-10 F2, Col-0 x HR-10 F2, Shahdara x Ws-2 F2, or a combination thereof.

In some embodiments, the population of polyploid seed comprises two, three, or more haplotypes of the same or related species of plant. In another embodiment, the population of polyploid seed comprises two, three, or more haplotypes of related species of plant. In some embodiments, related species of plant are species of plant within the same family. In other embodiments, related species of plant are species of plant within the same genus.

In some embodiments, the population of polyploid seed is a population of seed of the family Solanaceae comprising two, three, or more haplotypes from one or more species within the family Solanaceae. In some embodiments, the population of polyploid seed is a population of seed of the genus Solanum comprising two, three, or more haplotypes from one or more species within the genus Solanum. The population of polyploid seed of the genus Solanum may be a population of seed of any plant within the genus Solanum including, but not limited to, potato, tomato, and eggplant. In some variations, the two, three, or more haplotypes may be from any species or subspecies in the genus Solanum, including, but not limited to, Solanum chacoense, Solanum tuberosum, Solanum tuberosum ssp. andigena, Solanum tuberosum ssp. tuberosum, Solanum lycopersicum, Solanum pimpinellifolium, Solanum peruvianum, Solanum cheesmanii, Solanum galapagense, Solanum chilense, Solanum aethiopicum, Solanum quitoense, Solanum torvum, Solanum muricatum, Solanum betaceum, Solanum lycocarpum, Solanum scabrum, and Solanum spp. In certain embodiments, the population of polyploid seed is a population of potato seed comprising two, three, or more haplotypes from the same or related species of potato. In some variations, the population of potato seed comprises two, three, or more haplotypes of potato species or subspecies including, but not limited to, Solanum chacoense, Solanum tuberosum, Solanum tuberosum ssp. andigena, Solanum tuberosum ssp. tuberosum, Solanum stenotomum, Solanum phureja, Solanum goniocalyx, Solanum ajanhuiri, Solanum chaucha, Solanum juzepczukii, Solanum curtilobum, Solanum brevicaule, Solanum fendleri, Solanum demissum, and Solanum bulbocastanum.

In some embodiments, the population of polyploid seed comprises two, three, or more haplotypes of potato. In some variations, the two, three, or more haplotypes may be from different subspecies of Solanum tuberosum, including without limitation Solanum tuberosum ssp. andigena, Solanum tuberosum ssp. Tuberosum, related Solanum species, including without limitation Solanum chacoense, or Solanum tuberosum having introgressions from related Solanum species, including without limitation Solanum microdontum, Solanum berthaultii, Solanum tarijense, Solanum raphanifolium, Solanum verrucosum, and the like.

In some embodiments, the population of polyploid seed is a population of seed of the family Ericaceae comprising two, three, or more haplotypes from one or more species within the family Ericaceae. In some embodiments, the population of polyploid seed is a population of seed of the genus Vaccinium comprising two, three, or more haplotypes from one or more species within the genus Vaccinium. The population of polyploid seed of the genus Vaccinium may be a population of seed of any plant within the genus Vaccinium including, but not limited to, blueberry, cranberry, bilberry, lingonberry, cowberry, and huckleberry. In some variations, the two, three, or more haplotypes may be from any species or subspecies in the genus Vaccinium, including, but not limited to, Vaccinium darrowii, Vaccinium corymbosum, Vaccinium boreale, Vaccinium caesariense, Vaccinium caespitosum, Vaccinium elliottii, Vaccinium cereum, Vaccinium myrsinites, Vaccinium myrtilloides, Vaccinium macrocarpon, Vaccinium oxycoccos, Vaccinium microcarpum, Vaccinium erythrocarpum, Vaccinium ovatum, Vaccinium uliginosum, Vaccinium vitis-idaea, Vaccinium stamineum, Vaccinium caesium, Vaccinium calycinum, Vaccinium cespitosum, Vaccinium deliciosum, Vaccinium dentatum, Vaccinium membranaceum, Vaccinium myrtillus, Vaccinium ovalifolium, Vaccinium parvifolium, Vaccinium praestans, Vaccinium reticulatum, Vaccinium scoparium, Vaccinium koreanum, Vaccinium angustifolium, Vaccinium stenophyllum, Vaccinium fuscatum, Vaccinium atrococcum, Vaccinium pallidum, Vaccinium vacillans, Vaccinium tenellum, Vaccinium virgatum, Vaccinium ashei, Vaccinium moupinense, Vaccinium arboretum, Vaccinium crassifolium, and Vaccinium spp. In certain embodiments, the population of polyploid seed is a population of blueberry seed comprising two, three, or more haplotypes from the same or related species of blueberry. In some variations, the population of blueberry seed comprises two, three, or more haplotypes of blueberry species or subspecies including, but not limited to, Vaccinium darrowii, Vaccinium corymbosum, Vaccinium angustifolium, Vaccinium boreale, Vaccinium caesariense, Vaccinium corymbosum, Vaccinium darrowii, Vaccinium elliottii, Vaccinium formosum, Vaccinium fuscatum, Vaccinium atrococcum, Vaccinium hirsutum, Vaccinium myrsinites, Vaccinium myrtilloides, Vaccinium pallidum, Vaccinium simulatum, Vaccinium tenellum, Vaccinium virgatum, and Vaccinium ashei.

In some embodiments, the population of polyploid seed is a population of seed of the family Rosaceae comprising two, three, or more haplotypes from one or more species within the family Rosaceae. In some embodiments, the population of polyploid seed is a population of seed of the genus Rubus comprising two, three, or more haplotypes from one or more species within the genus Rubus. The population of polyploid seed of the genus Rubus may be a population of seed of any plant within the genus Rubus including, but not limited to, blackberry, raspberry, dewberry, caneberry, loganberry, boysenberry, marionberry, and tayberry. In some variations, the two, three, or more haplotypes may be from any species or subspecies in the genus Rubus, including, but not limited to, Rubus fruticosus, Rubus aboriginum, Rubus allegheniensis, Rubus arcticus, Rubus arizonensis, Rubus armeniacus, Rubus caesius, Rubus canadensis, Rubus chamaemorus, Rubus coreanus, Rubus cuneifolius, Rubus depavitus, Rubus flagellaris, Rubus geoides, Rubus glaucifolius, Rubus hispidus, Rubus hochstetterorum, Rubus idaeus, Rubus invisus, Rubus laciniatus, Rubus lasiococcus, Rubus leucodermis, Rubus occidentalis, Rubus odoratus, Rubus parviflorus, Rubus parvifolius, Rubus pectinellus, Rubus pensilvanicus, Rubus phoenicolasius, Rubus pubescens, Rubus reflexus, Rubus saxatilis, Rubus spectabilis, Rubus strigosus, Rubus trifidus, Rubus ursinus, and Rubus spp. In certain embodiments, the population of polyploid seed is a population of blackberry seed comprising two, three, or more haplotypes from the same or related species of blackberry. In some variations, the population of blackberry seed comprises two, three, or more haplotypes of blackberry species or subspecies including, but not limited to, Rubus fruticosus, Rubus plicatus, Rubus armeniacus, Rubus laciniatus, Rubus occidentalis, Rubus allegheniensis, Rubus canadensis, Rubus cuneifolius, Rubus hochstetterorum, Rubus leucodermis, Rubus odoratus, Rubus pensilvanicus, Rubus pubescens, Rubus trifidus, and Rubus ursinus.

In some embodiments, the population of polyploid seed is a population of seed of the family Poaceae comprising two, three, or more haplotypes from one or more species within the family Poaceae. The population of polyploid seed of the family Poaceae may be a population of seed of any plant within the family Poaceae including, but not limited to, maize, wheat, rice, barley, millet, sugar cane, oats, rye, bamboo, ryegrass, lawn grasses, ornamental grasses, and the like. In some embodiments, the population of polyploid seed comprises two, three, or more haplotypes of maize. In some variations, the two, three, or more haplotypes may be from different subspecies of maize. In some embodiments, the population of polyploid seed is a population of seed of the genus Zea comprising two, three, or more haplotypes from one or more species within the genus Zea. The population of polyploid seed of the genus Zea may be a population of seed of any plant within the genus Zea including, but not limited to, maize and teosinte. In some variations, the two, three, or more haplotypes may be from any species or subspecies in the genus Zea including, but not limited to, Zea mays, Zea diploperennis, Zea nicaraguensis, Zea perennis, and Zea spp. In certain embodiments, the population of polyploid seed is a population of maize seed comprising two, three, or more haplotypes from the same or related species of maize or teosinte including, but not limited to, the species of Zea described herein.

In some embodiments, the population of polyploid seed is a population of seed of the family Musaceae comprising two, three, or more haplotypes from one or more species within the family Musaceae. In some embodiments, the population of polyploid seed is a population of seed of the genus Musa comprising two, three, or more haplotypes from one or more species within the genus Musa. The population of polyploid seed of the genus Musa may be a population of seed of any plant within the genus Musa including, but not limited to, bananas, plantains, red bananas, latundan bananas, and Cavendish bananas. In some variations, the two, three, or more haplotypes may be from any species or subspecies in the genus Musa, including, but not limited to, Musa acuminata, Musa acuminata subsp. zebrine, Musa balbisiana, Musa basjoo, Musa velutina, Musa yunnanensis, Musa coccinea, Musa schizocarpa, Musa x troglodytarum, Musa x paradisiaca, and Musa spp. In certain embodiments, the population of polyploid seed is a population of banana or plantain seed comprising two, three, or more haplotypes from the same or related species of banana or plantain including, but not limited to, the species of Musa described herein.

### Genetic Uniformity, Ploidy, and Origin

In some embodiments, at least 50% of the population of polyploid seed comprising two, three, or more haplotypes are genetically uniform. In some variations, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid seed comprising two, three, or more haplotypes are genetically uniform. In some embodiments, at least 50% of the population of polyploid seed produced are genetically uniform, wherein the polyploid seed comprises three or more haplotypes. In some variations, at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid seed produced are genetically uniform, wherein the polyploid seed comprises three or more haplotypes. In some embodiments, the population of polyploid seed has an average pairwise genetic uniformity of at least 80% as measured by the Jaccard similarity coefficient. In some variations, the population of polyploid seed has an average pairwise genetic uniformity of at least 80%, at least 85%, at least 90%, at least 91%, at least 92%, at least 93%, at least 94%, at least 95%, at least 96%, at least 97%, at least 98%, at least 99%, or 100% as measured by the Jaccard similarity coefficient. In one variation, the population of polyploid seed has an average pairwise genetic uniformity of at least 85% as measured by the Jaccard similarity coefficient. In another variation, the population of polyploid seed has an average pairwise identity of at least 90% as measured by the Jaccard similarity coefficient.

In some embodiments, the population of polyploid seed comprises a subpopulation of genetically uniform polyploid seed in an amount of at least 50% of the total number of seeds. In some variations, the population of polyploid seed comprises a subpopulation of genetically uniform polyploid seed in an amount of at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the total number of seeds. In some embodiments, each pair of the subpopulation of genetically uniform seed has a pairwise identity of at least about 90% as measured by the Jaccard similarity coefficient. In some embodiments, each pair of the subpopulation of genetically uniform seed has a pairwise identity of at least about 90%, at least about 95%, at least about 96%, at least about 97%, at least about 98%, at least about 99%, or about 100% as measured by the Jaccard similarity coefficient.

In some embodiments, the population of polyploid seed or the subpopulation of genetically uniform polyploid seed comprising two, three, or more haplotypes is triploid, tetraploid, pentaploid, hexaploid, heptaploid, or octoploid. In other embodiments, the population of polyploid seed or the subpopulation of genetically uniform polyploid seed comprising two, three, or more haplotypes has a ploidy of 9n, 10n, 11n, 12n, 13n, 14n, 15n, 16n, or higher.

In some embodiments, the population of polyploid seed was obtained from a single plant or a set of plants such as, for example, a set of F1 hybrids. In some embodiments, the population of polyploid seed was obtained from a single plant. In other embodiments, the population of polyploid seed was obtained from a set of F1 hybrids. In some variations, the population of polyploid seed was obtained from a set of two, three, four, five, 10, 20, 50, 100, or more F1 hybrids. In some additional variations, the population of polyploid seed was obtained from a set of genetically uniform plants, e.g., a set of F1 hybrids derived from the same inbred parents. In yet additional variations, the population of polyploid seed was obtained from a set of two, three, four, five, 10, 20, 50, 100, or more genetically uniform plants, e.g., genetically uniform F1 hybrids. In certain embodiments, the genetically uniform set of plants (e.g., the genetically uniform set of F1 hybrids) has an average pairwise genetic uniformity of at least 80%, at least 85%, at least 90%, at least 91%, at least 92%, at least 93%, at least 94%, at least 95%, at least 96%, at least 97%, at least 98%, at least 99%, or 100% as measured by the Jaccard similarity coefficient. In certain embodiments, each pair of the genetically uniform set of plants (e.g., the genetically uniform set of F1 hybrids) has a pairwise identity of at least about 90%, at least about 95%, at least about 96%, at least about 97%, at least about 98%, at least about 99%, or about 100% as measured by the Jaccard similarity coefficient.

Methods of measuring genetic uniformity are known in the art. One exemplary method of measuring genetic uniformity is by using the Jaccard similarity coefficient, also known as the Jaccard index or Jaccard similarity index. In the context of molecular plant genetics, the Jaccard index, Jaccard similarity index, or Jaccard similarity coefficient (Jaccard, P. (1908) Nouvelles Recherches sur la Distribution Florale. Bulletin de la Société Vaudoise des Sciences Naturelles. Vol. 44), is commonly applied to quantify the pairwise genetic similarity or uniformity of plants based on the presence or absence of shared alleles at loci spread throughout the genome. Exemplary methods of using the Jaccard similarity coefficient to measure genetic uniformity between two plants are described in Example 1 below and, for example, in Paz and SVeilleux (1997. Genetic diversity based on randomly amplified polymorphic DNA (RAPD) and its relationship with the performance of diploid potato hybrids. Journal of the American Society for Horticultural Sci. 122(6): 740-747), Vosman et al. (2004. The establishment of ‘essential derivation’ among rose varieties, using AFLP. Theoretical and Applied Genetics. 109: 1718-1725), Noli et al. (2013. Criteria for the definition of similarity thresholds for identifying essentially derived varieties. Plant Breeding. 132(6): 525-531), Vijayakumar et al. (2021. High temperature induced changes in quality and yield parameters of tomato (Solanum lycopersicum L.) and similarity coefficients among genotypes using SSR markers. Heliyon. 7(2)), and Dalamu et al. (2023. Genetic Diversity and Population Structure Analyses Using Simple Sequence Repeat Markers and Phenotypic Traits in Native Potato Collection in India. Potato Research: 1-25). The Jaccard similarity coefficient is defined as the ratio of the number of shared items to the total number of distinct items in the two sets. In the context of molecular plant genetics, it quantifies the proportion of shared alleles between two plants. The formula for calculating the Jaccard similarity coefficient is:

$$J ( A , B ) = \vert A ⋂ B \vert / \vert A ⋃ B \vert$$

Where A represents the set of unique alleles without duplication in one plant, B represents the set of unique alleles without duplication in the other plant, |A∩B| represents the number of shared alleles (the cardinality of the intersection) between the plants, and |A∪B| represents the number of distinct alleles (the cardinality of the union) between the plants. This formula computes the cardinality of the intersection (common elements) of two sets (the shared alleles) divided by the cardinality of the union (all alleles) of the two sets (all distinct alleles present). The resulting value of the Jaccard similarity coefficient ranges from 0 to 1, where 0 indicates no shared alleles, and 1 indicates complete uniformity. The average pairwise genetic uniformity of the populations was calculated as the average Jaccard similarity of all possible pairs of plants within the population. In the context of genetic pairwise similarity estimations, the size of A should be the same as, or very close to the size of B to avoid misinterpretation.

### Genetic Modifications

In some embodiments, the polyploid seed (e.g., the subpopulation of genetically uniform polyploid seed) comprises one or more genetic modifications. Genetic modifications may be generated by modification of any nucleic acid sequence or genetic element by insertion, deletion, or substitution of one or more nucleotides in a nucleic acid molecule. This can occur by a replacement of at least one nucleotide, a deletion of at least one nucleotide, an insertion of at least one nucleotide, a chemical alteration of at least one nucleotide, or a combination thereof as long as the result is a detectable (e.g., by PCR, DNA sequencing, chromatography, etc.) change of nucleotide sequence compared to the sequence of the nucleic acid molecule prior to modification. Such modifications can be achieved by any of several well-known methods known in the art including, but not limited to, random mutagenesis, genome editing, insertion of a recombinant nucleic acid, crossing of an unmodified plant with a modified plant to introduce the modification of the modified plant into the unmodified plant, and the like. A genetic modification may be naturally occurring or non-naturally occurring.

The genetic modifications described herein may be present in any known genetic element including, but not limited to, protein-coding sequences, non-protein-coding sequences, promoter regions, 5′ untranslated leaders, genes, exons, introns, poly-A signal sequences, 3′ untranslated regions, regions encoding small RNAs (such as microRNAs and small-interfering RNAs), and any other sequences that affect transcription or translation of one or more nucleic acid sequences. In some embodiments, genetic modifications may include, but are not limited to, modifying or replacing nucleotide sequences of interest (such as a regulatory elements), gene disruption, gene knockout, gene knockdown, gene knock-in, gene silencing (including, e.g., by expressing an inverted repeat into a gene of interest), RNA interference (including, e.g., by insertion and/or expression of an RNA interference construct), modification of methylation status, modification of splicing sites, introducing alternate splicing sites, or any combination thereof. As used herein, gene disruption refers to the alteration or insertion of a sequence into a gene or locus that results in decreased expression (including non-expression or altered activity) of a functional protein gene product. A gene disruption may be achieved by introduction of a genetic modification in a protein-coding sequence, including, but not limited to, as a missense or nonsense mutation, or an insertion, deletion, or substitution. As used herein, a knockout is a genetic modification wherein a gene or gene product has been rendered completely inoperative. A knockout of a gene product may be achieved by introduction of a genetic modification in a protein-coding sequence of a gene or any non-protein-coding or regulatory sequence described herein. As used herein, a knockdown is a genetic modification wherein a gene or gene product has been rendered partially inoperative. A knockdown of a gene product may be achieved by introduction of a genetic modification in a protein-coding sequence of a gene or in a non-protein-coding or regulatory sequence, or insertion of a trans-acting element, such as a construct that expresses an inverted repeat of the gene product or a construct that expresses a DNA- or RNA-binding protein such as a transcriptional repressor which may include, for example, a deactivated targeted nuclease such as deactivated Cas9 (dCas9). As used herein, knock-in represents the replacement or insertion of a DNA sequence at a specific DNA locus in a cell. Knock-ins may include, but are not limited to, specific insertion of a heterologous amino acid coding sequence in a coding region of a gene, an insertion of a transcriptional regulatory element in a genetic locus, or any of several methods of inserting a DNA sequence into a cell that are known to one of ordinary skill in the art.

In certain embodiments, the polyploid seed (e.g., the subpopulation of genetically uniform polyploid seed) comprises one or more genetic modifications resulting in decreased expression (including non-expression or altered activity) of a gene product of a genomic locus. In some embodiments, genetic modifications resulting in decreased expression (including non-expression or altered activity) of a gene product or locus may include, but are not limited to, modification of an enhancer, modification of a promoter, modification of a 5′ untranslated leader, modification of a coding region, modification of a non-coding region, insertion and/or expression of an RNA interference construct that targets an mRNA, modification of a region encoding a small RNA, modification of methylation status of a genomic locus, expression of a repressor protein that targets a DNA or mRNA sequence, and any other sequences that affect transcription or translation of one or more nucleic acid sequences. In some embodiments, genetic modifications resulting in decreased expression (including non-expression or altered activity) of a gene product or locus may include, but are not limited to, modifying or replacing nucleotide sequences of interest (such as a regulatory elements), gene disruption, gene knockout, gene knockdown, gene knock-in, gene silencing (including, e.g., by inserting and/or expressing an inverted repeat into a gene of interest), RNA interference (including, e.g., by insertion and/or expression of an RNA interference construct), expression of a repressor protein (e.g. dCas9), modification of methylation status of gene loci, modification of splicing sites, introducing alternate splicing sites, or any combination thereof. In some variations, the genetic modification is positioned in the first 70%, the first 60%, the first 50%, the first 40%, the first 30%, the first 20%, or the first 10% of the nucleotides of the coding sequence of the genomic locus following the start codon in the 3′ direction. In certain variations, the genetic modification is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction.

In some embodiments, one or more genetic modifications each independently comprise an insertion, a deletion, one or more nucleotide changes, or an inversion that results in decreased expression of the one or more genomic loci (e.g., MiMe loci). In some variations, the insertion, the deletion, the one or more nucleotide changes, or the inversion eliminates expression (e.g., eliminates activity) of the genomic locus. In some variations, the insertion, the deletion, the one or more nucleotide changes, or the inversion is positioned in the first 70%, the first 60%, the first 50%, the first 40%, the first 30%, the first 20%, or the first 10% of the nucleotides of the coding sequence of the genomic locus following the start codon in the 3′ direction. In certain variations, the insertion, the deletion, the one or more nucleotide changes, or the inversion is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction. In some embodiments, the insertion, the deletion, the one or more nucleotide changes, or the inversion eliminates expression (e.g., eliminates activity) of the genomic locus. In some variations, the insertion, the deletion, the one or more nucleotide changes, or the inversion results in a premature stop codon present in the first 70%, the first 60%, the first 50%, the first 40%, the first 30%, the first 20%, or the first 10% of the nucleotides of the coding sequence of the MiMe locus following the start codon in the 3′ direction, thereby eliminating expression (e.g., activity) of the genomic locus. In some variations, the insertion, the deletion, the one or more nucleotide changes, or the inversion results in a premature stop codon present in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction, thereby eliminating expression (e.g., activity) of the genomic locus.

In some embodiments, the one or more genetic modifications comprise one or more polynucleotide sequences selected from the group consisting of SEQ ID NOs: 448-466, 468-471, 474-491, and 493-496. In certain embodiments, the one or more genetic modifications comprise one or more polynucleotide sequences each having at least 80%, at least 85%, at least 90%, at least 95%, at least 96%, at least 97%, at least 98%, or at least 99% identity or complementarity to a nucleotide sequence selected from the group consisting of SEQ ID NOs: 448-466, 468-471, 474-491, and 493-496.

### MiMe Loci, MiMe Genotypes, and MiMe Components

In some embodiments, the polyploid seed (e.g., the subpopulation of genetically uniform polyploid seed) comprises one or more genetic modifications resulting in decreased expression (including non-expression or altered activity) of one or more MiMe loci. In some variations, the polyploid seed (e.g., the subpopulation of genetically uniform polyploid seed) comprises one or more genetic modifications resulting in a decreased amount of a functional gene product encoded at one or more MiMe loci. The gene products at the MiMe loci may include, but are not limited to, nucleic acids (e.g. RNA), post-transcriptionally modified nucleic acids (e.g. spliced RNA, poly-adenylated mRNA), proteins (e.g. enzymes, structural proteins, etc.), and post-translationally modified proteins (e.g. glycoproteins, lipoproteins, etc.). The function of the gene product at the MiMe locus refers to the wild-type, unmodified function of the gene product. The decreased expression of a MiMe locus may refer to a decrease in the total amount of a gene product encoded at a MiMe locus present in a cell (e.g. a decrease in the amount of a protein, including up to no detectable expression) or to a decrease in the amount of a functional gene product encoded at a MiMe locus present in a cell (e.g. a decrease in the percentage of proteins with wild-type function, or an increase in the percentage of proteins with altered activity). In some embodiments, the one or more genetic modifications resulting in decreased expression of one or more MiMe loci may include, but are not limited to, modification of an enhancer in the Mime loci, modification of a promoter of the MiMe loci, modification of a coding region in the MiMe loci, modification of methylation status of the MiMe loci, expression of a repressor protein that targets the DNA or an mRNA of the MiMe loci, and expression of an RNA interference construct that targets an mRNA from the MiMe loci. In some embodiments, the polyploid seed (e.g., the subpopulation of genetically uniform polyploid seed) may comprise one or more genetic modifications resulting in non-expression of one or more MiMe loci. In some embodiments, the polyploid seed (e.g., the subpopulation of genetically uniform polyploid seed) may comprise one or more genetic modifications resulting in decreased expression (including non-expression or altered activity) of a combination of two or more MiMe loci.

In some embodiments, the polyploid seed comprises one or more genetic modifications resulting in decreased expression of one or more MiMe loci. In other embodiments, the polyploid seed (e.g., the subpopulation of genetically uniform polyploid seed) comprises one or more genetic modifications resulting in decreased expression of two or more MiMe loci. In yet another embodiment, the polyploid seed (e.g., the subpopulation of genetically uniform polyploid seed) comprises one or more genetic modifications resulting in decreased expression of three or more MiMe loci. In some variations, the MiMe loci may include, but are not limited to, REC8, OSD1 (e.g., OSD1-1, OSD1-2, and/or OSD1-3 in maize), CYCA1, TDM1, PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, SWITCH1/DYAD, PS1, PS1-LIKE PROTEIN, JASON (e.g., JASON-1 and/or JASON-2 in maize), PC1, PC2, and FC. In one variation, the polyploid seed (e.g., the subpopulation of genetically uniform polyploid seed) comprises one or more genetic modifications resulting in decreased expression of REC8. In a second variation, the polyploid seed (e.g., the subpopulation of genetically uniform polyploid seed) comprises one or more genetic modifications resulting in decreased expression of OSD1 (e.g., OSD1-1, OSD1-2, and/or OSD1-3 in maize). In a third variation, the polyploid seed (e.g., the subpopulation of genetically uniform polyploid seed) comprises one or more genetic modifications resulting in decreased expression of PAIR1 (e.g., PAIR1-1 and/or PAIR1-2 in maize). In a fourth variation, the polyploid seed (e.g., the subpopulation of genetically uniform polyploid seed) comprises one or more genetic modifications resulting in decreased expression of SPO11-1, SPO11-2, or a combination thereof. In a fifth variation, the polyploid seed (e.g., the subpopulation of genetically uniform polyploid seed) comprises one or more genetic modifications resulting in decreased expression of REC8 and SPO11-1. In a sixth variation, the polyploid seed (e.g., the subpopulation of genetically uniform polyploid seed) comprises one or more genetic modifications resulting in decreased expression of REC8 and OSD1 (e.g., OSD1-1, OSD1-2, and/or OSD1-3 in maize). In a seventh variation, the polyploid seed (e.g., the subpopulation of genetically uniform polyploid seed) comprises one or more genetic modifications resulting in decreased expression of REC8 and PAIR1 (e.g., PAIR1-1 and/or PAIR1-2 in maize). In an eighth variation, the polyploid seed (e.g., the subpopulation of genetically uniform polyploid seed) comprises one or more genetic modifications resulting in decreased expression of OSD1 (e.g., OSD1-1, OSD1-2, and/or OSD1-3 in maize) and SPO11-1. In a ninth variation, the polyploid seed (e.g., the subpopulation of genetically uniform polyploid seed) comprises one or more genetic modifications resulting in decreased expression of OSD1 (e.g., OSD1-1, OSD1-2, and/or OSD1-3 in maize) and PAIR1 (e.g., PAIR1-1 and/or PAIR1-2 in maize). In a tenth variation, the polyploid seed (e.g., the subpopulation of genetically uniform polyploid seed) comprises one or more genetic modifications resulting in decreased expression of REC8, OSD1 (e.g., OSD1-1, OSD1-2, and/or OSD1-3 in maize), and PAIR1 (e.g., PAIR1-1 and/or PAIR1-2 in maize). In an eleventh variation, the polyploid seed (e.g., the subpopulation of genetically uniform polyploid seed) comprises one or more genetic modifications resulting in decreased expression of REC8, OSD1 (e.g., OSD1-1, OSD1-2, and/or OSD1-3 in maize), and SPO11-1. In a twelfth variation, the polyploid seed (e.g., the subpopulation of genetically uniform polyploid seed) comprises one or more genetic modifications resulting in decreased expression of PS1 and SPO11-1. In a thirteenth variation, the polyploid seed (e.g., the subpopulation of genetically uniform polyploid seed) comprises one or more genetic modifications resulting in decreased expression of PS1 and SY3. In a fourteenth variation, the polyploid seed (e.g., the subpopulation of genetically uniform polyploid seed) is maize seed and comprises one or more genetic modifications resulting in decreased expression of OSD1 (e.g., OSD1-1, OSD1-2, and/or OSD1-3 in maize), SPO11-1, and REC8. In a fifteenth variation, the polyploid seed (e.g., the subpopulation of genetically uniform polyploid seed) is potato seed and comprises one or more genetic modifications resulting in decreased expression of PS1-LIKE PROTEIN and SPO11-1. In a sixteenth variation, the polyploid seed (e.g., the subpopulation of genetically uniform polyploid seed) is potato seed and comprises one or more genetic modifications resulting in decreased expression of PS1-LIKE PROTEIN and SY3. In a seventeenth variation, the polyploid seed (e.g., the subpopulation of genetically uniform polyploid seed) is potato seed and comprises one or more genetic modifications resulting in altered activity of TDM1 (e.g., a dominant negative, constitutively active or null mutant of TDM1). In an eighteenth variation, the polyploid seed (e.g., the subpopulation of genetically uniform polyploid seed) is potato seed and comprises one or more genetic modifications resulting in decreased expression of PS1-LIKE PROTEIN and SPO11-1. In a nineteenth variation, the polyploid seed (e.g., the subpopulation of genetically uniform polyploid seed) is potato and comprises one or more genetic modifications resulting in decreased expression of PS1-LIKE PROTEIN and SY3. In a twentieth variation, the polyploid seed (e.g., the subpopulation of genetically uniform polyploid seed) comprises one or more genetic modifications resulting in decreased expression of REC8, OSD1 (e.g., OSD1-1, OSD1-2, and/or OSD1-3 in maize), SPO11-1, and PAIR1 (e.g., PAIR1-1 and/or PAIR1-2 in maize). In a twenty-first variation, the polyploid seed (e.g., the subpopulation of genetically uniform polyploid seed) comprises one or more genetic modifications resulting in decreased expression of PS1 or PS1-LIKE PROTEIN, SY3, and SPO11-1. The polyploid seed (e.g., the subpopulation of genetically uniform polyploid seed) may comprise one or more genetic modifications resulting in decreased expression of any combination of MiMe loci described herein or known in the art. In some embodiments, the polyploid seed (e.g., the subpopulation of genetically uniform polyploid seed) may comprise one or more genetic modifications resulting in non-expression of any combination of MiMe loci described here or known in the art. In further embodiments, the polyploid seed (e.g., the subpopulation of genetically uniform polyploid seed) may comprise one or more genetic modifications resulting in decreased expression (including non-expression or altered activity) of a combination of two or more MiMe loci described here or known in the art.

In some embodiments, the polyploid seed (e.g., the subpopulation of genetically uniform polyploid seed) comprises one or more genetic modifications resulting in decreased expression of REC8, SWITCH1/DYAD, or a combination thereof. In some variations, the polyploid seed (e.g., the subpopulation of genetically uniform polyploid seed) comprises one or more genetic modifications resulting in decreased expression of REC8, SWITCH1/DYAD, or a combination thereof and one or more genetic modifications resulting in decreased expression of one or more additional MiMe loci. In additional variations, the polyploid seed (e.g., the subpopulation of genetically uniform polyploid seed) comprises one or more genetic modifications resulting in decreased expression of REC8, SWITCH1/DYAD, or a combination thereof and one or more genetic modifications resulting in decreased expression of one or more additional MiMe loci which may include, but are not limited to, OSD1 (e.g., OSD1-1, OSD1-2, and/or OSD1-3 in maize), CYCA1, TDM1, PC1, PC2, and FC. In yet additional variations, the polyploid seed (e.g., the subpopulation of genetically uniform polyploid seed) comprises one or more genetic modifications resulting in decreased expression of REC8, SWITCH1/DYAD, or a combination thereof; one or more genetic modifications resulting in decreased expression of OSD1 (e.g., OSD1-1, OSD1-2, and/or OSD1-3 in maize), CYCA1, TDM1, PC1, PC2, FC, or any combination thereof; and one or more genetic modifications resulting in decreased expression of one or more additional MiMe loci, which may include, but are not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the polyploid seed (e.g., the subpopulation of genetically uniform polyploid seed) comprises one or more genetic modifications resulting in decreased expression of one or more MiMe loci which may include, but are not limited to, OSD1 (e.g., OSD1-1, OSD1-2, and/or OSD1-3 in maize), CYCA1, TDM1, PC1, PC2, and FC. In some variations, the polyploid seed (e.g., the subpopulation of genetically uniform polyploid seed) comprises one or more genetic modifications resulting in decreased expression of OSD1 (e.g., OSD1-1, OSD1-2, and/or OSD1-3 in maize), CYCA1, TDM1, PC1, PC2, FC, or any combination thereof and further comprises one or more genetic modifications resulting in decreased expression of one or more additional MiMe loci, which may include, but are not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the polyploid seed (e.g., the subpopulation of genetically uniform polyploid seed) comprises one or more genetic modifications resulting in decreased expression of one or more MiMe loci which may include, but are not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4. In some variations, the polyploid seed (e.g., the subpopulation of genetically uniform polyploid seed) comprises one or more genetic modifications resulting in decreased expression of PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, or any combination thereof, and further comprises one or more genetic modifications resulting in decreased expression of one or more additional MiMe loci, which may include, but are not limited to PS1, PS1-LIKE PROTEIN, and JASON (e.g., JASON-1 and/or JASON-2 in maize).

In some embodiments, each of the one or more MiMe loci encodes a protein of a MiMe component as described herein. In certain embodiments, each of the one or more MiMe loci encodes a protein having an amino acid sequence selected from the group consisting of SEQ ID NOs: 1-119. In some variations, each of the one or more MiMe loci encodes a protein having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to an amino acid sequence selected from the group consisting of SEQ ID NOs: 1-119.

In some embodiments, the population of polyploid seed or the subpopulation of genetically uniform polyploid seed has a complete MiMe genotype. In alternative embodiments, the population of polyploid seed or the subpopulation of genetically uniform polyploid seed has a partial MiMe genotype. In still other embodiments, the population of polyploid seed or the subpopulation of genetically uniform polyploid seed has a partially-complemented MiMe genotype. In certain embodiments, the complete, partial, or partially-complemented MiMe genotype comprises one or more genetic modifications resulting in decreased expression of one or more MiMe loci. In other embodiments, the complete, partial, or partially-complemented MiMe genotype comprises one or more genetic modifications resulting in decreased expression of two or more MiMe loci. In yet another embodiment, the complete, partial, or partially-complemented MiMe genotype comprises one or more genetic modifications resulting in decreased expression of three or more MiMe loci. In some variations, the MiMe loci may include, but are not limited to, REC8, OSD1 (e.g., OSD1-1, OSD1-2, and/or OSD1-3 in maize), CYCA1, TDM1, PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, SWITCH1/DYAD, PS1, PS1-LIKE PROTEIN, JASON (e.g., JASON-1 and/or JASON-2 in maize), PC1, PC2, and FC. In one variation, the complete, partial, or partially-complemented MiMe genotype comprises one or more genetic modifications resulting in decreased expression of REC8. In a second variation, the complete, partial, or partially-complemented MiMe genotype comprises one or more genetic modifications resulting in decreased expression of OSD1 (e.g., OSD1-1, OSD1-2, and/or OSD1-3 in maize). In a third variation, the complete, partial, or partially-complemented MiMe genotype comprises one or more genetic modifications resulting in decreased expression of PAIR1 (e.g., PAIR1-1 and/or PAIR1-2 in maize). In a fourth variation, the complete, partial, or partially-complemented MiMe genotype comprises one or more genetic modifications resulting in decreased expression of SPO11-1, SPO11-2, or a combination thereof. In a fifth variation, the complete, partial, or partially-complemented MiMe genotype comprises one or more genetic modifications resulting in decreased expression of REC8 and SPO11-1. In a sixth variation, the complete, partial, or partially-complemented MiMe genotype comprises one or more genetic modifications resulting in decreased expression of REC8 and OSD1 (e.g., OSD1-1, OSD1-2, and/or OSD1-3 in maize). In a seventh variation, the complete, partial, or partially-complemented MiMe genotype comprises one or more genetic modifications resulting in decreased expression of REC8 and PAIR1 (e.g., PAIR1-1 and/or PAIR1-2 in maize). In an eighth variation, the complete, partial, or partially-complemented MiMe genotype comprises one or more genetic modifications resulting in decreased expression of OSD1 (e.g., OSD1-1, OSD1-2, and/or OSD1-3 in maize) and SPO11-1. In a ninth variation, the complete, partial, or partially-complemented MiMe genotype comprises one or more genetic modifications resulting in decreased expression of OSD1 (e.g., OSD1-1, OSD1-2, and/or OSD1-3 in maize) and PAIR1 (e.g., PAIR1-1 and/or PAIR1-2 in maize). In a tenth variation, the complete, partial, or partially-complemented MiMe genotype comprises one or more genetic modifications resulting in decreased expression of REC8, OSD1 (e.g., OSD1-1, OSD1-2, and/or OSD1-3 in maize), and PAIR1 (e.g., PAIR1-1 and/or PAIR1-2 in maize). In an eleventh variation, the complete, partial, or partially-complemented MiMe genotype comprises one or more genetic modifications resulting in decreased expression of REC8, OSD1 (e.g., OSD1-1, OSD1-2, and/or OSD1-3 in maize), and SPO11-1. In a twelfth variation, the complete, partial, or partially-complemented MiMe genotype comprises one or more genetic modifications resulting in decreased expression of PS1 and SPO11-1. In a thirteenth variation, the complete, partial, or partially-complemented MiMe genotype comprises one or more genetic modifications resulting in decreased expression of PS1 and SY3. In a fourteenth variation, the population of polyploid seed is maize seed, and the complete, partial, or partially-complemented MiMe genotype comprises one or more genetic modifications resulting in decreased expression of OSD1 (e.g., OSD1-1, OSD1-2, and/or OSD1-3), SPO11-1, and REC8. In a fifteenth variation, the population of polyploid seed is potato seed, and the complete, partial, or partially-complemented MiMe genotype comprises one or more genetic modifications resulting in decreased expression of PS1-LIKE PROTEIN and SPO11-1. In a sixteenth variation, the population of polyploid seed is potato seed, and the complete, partial, or partially-complemented MiMe genotype comprises one or more genetic modifications resulting in decreased expression of PS1-LIKE PROTEIN and SY3. In a seventeenth variation, the population of polyploid seed is potato seed, and the complete, partial, or partially-complemented MiMe genotype comprises one or more genetic modifications resulting in altered activity of TDM1 (e.g., a dominant negative, constitutively active or null mutant of TDM1). In an eighteenth variation, the polyploid seed is potato seed and the complete, partial, or partially-complemented MiMe genotype comprises one or more genetic modifications resulting in decreased expression of PS1-LIKE PROTEIN and SPO11-1. In a nineteenth variation, the polyploid seed is potato seed and the complete, partial, or partially-complemented MiMe genotype comprises one or more genetic modifications resulting in decreased expression of PS1-LIKE PROTEIN and SY3. In a twentieth variation, the complete, partial, or partially-complemented MiMe genotype comprises one or more genetic modifications resulting in decreased expression of REC8, OSD1 (e.g., OSD1-1, OSD1-2, and/or OSD1-3 in maize), SPO11-1, and PAIR1 (e.g., PAIR1-1 and/or PAIR1-2 in maize). In a twenty-first variation, the complete, partial, or partially-complemented MiMe genotype comprises one or more genetic modifications resulting in decreased expression of PS1 or PS1-LIKE PROTEIN, SY3, and SPO11-1. The complete, partial, or partially-complemented MiMe genotype may comprise one or more genetic modifications resulting in decreased expression of any combination of MiMe loci described herein or known in the art, wherein a plant that has the complete MiMe genotype exhibits a MiMe phenotype. In some embodiments, the complete, partial, or partially-complemented MiMe genotype may comprise one or more genetic modifications resulting in non-expression of any combination of MiMe loci described here or known in the art wherein a plant that has the complete MiMe genotype exhibits a MiMe phenotype. In further embodiments, the complete, partial, or partially-complemented MiMe genotype may comprise one or more genetic modifications resulting in decreased expression (including non-expression or altered activity) of a combination of two or more MiMe loci described here or known in the art, wherein a plant that has the complete MiMe genotype exhibits a MiMe phenotype. Specific examples of complete MiMe genotypes are shown in Table 10.

In some embodiments, the complete, partial, or partially-complemented MiMe genotype comprises one or more genetic modifications resulting in decreased expression of REC8, SWITCH1/DYAD, or a combination thereof. In some variations, the complete, partial, or partially-complemented MiMe genotype comprises one or more genetic modifications resulting in decreased expression of REC8, SWITCH1/DYAD, or a combination thereof and one or more genetic modifications resulting in decreased expression of one or more additional MiMe loci. In additional variations, the complete, partial, or partially-complemented MiMe genotype comprises one or more genetic modifications resulting in decreased expression of REC8, SWITCH1/DYAD, or a combination thereof and one or more genetic modifications resulting in decreased expression of one or more additional MiMe loci, including, but not limited to, OSD1 (e.g., OSD1-1, OSD1-2, and/or OSD1-3 in maize), CYCA1, TDM1, PC1, PC2, and FC. In yet additional variations, the complete, partial, or partially-complemented MiMe genotype comprises one or more genetic modifications resulting in decreased expression of REC8, SWITCH1/DYAD, or a combination thereof; one or more genetic modifications resulting in decreased expression of OSD1 (e.g., OSD1-1, OSD1-2, and/or OSD1-3 in maize), CYCA1, TDM1, PC1, PC2, FC, or any combination thereof; and one or more genetic modifications resulting in decreased expression of one or more additional MiMe loci, including, but not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the complete, partial, or partially-complemented MiMe genotype comprises one or more genetic modifications resulting in decreased expression of one or more MiMe loci including, but not limited to, OSD1 (e.g., OSD1-1, OSD1-2, and/or OSD1-3 in maize), CYCA1, TDM1, PC1, PC2, and FC. In some variations, the complete, partial, or partially-complemented MiMe genotype comprises one or more genetic modifications resulting in decreased expression of OSD1 (e.g., OSD1-1, OSD1-2, and/or OSD1-3 in maize), CYCA1, TDM1, PC1, PC2, FC, or any combination thereof and further comprises one or more genetic modifications resulting in decreased expression of one or more additional MiMe loci, including, but not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4.

In some embodiments, the complete, partial, or partially-complemented MiMe genotype comprises one or more genetic modifications resulting in decreased expression of one or more MiMe loci including, but not limited to, PAIR1 (e.g., PAIR1-1 and/or PAIR1-2 in maize), SPO11-1, SPO11-2, PRD1, PRD2, DFO, MTOPVIB, DSY1, SY1, SY2, SY3, and SY4. In some variations, the complete, partial, or partially-complemented MiMe genotype comprises one or more genetic modifications resulting in decreased expression of PAIR1 (e.g., PAIR1-1 and/or PAIR1-2 in maize), SPO11-1, SPO11-2, PRD1, PRD2, DFO, MTOPVIB, DSY1, SY1, SY2, SY3, SY4, or any combination thereof, and further comprises one or more genetic modifications resulting in decreased expression of one or more additional MiMe loci, which may include, but are not limited to PS1, PS1-LIKE PROTEIN, and JASON (e.g., JASON-1 and/or JASON-2 in maize).

In some embodiments, the population of polyploid seed or the subpopulation of genetically uniform polyploid seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.

In other embodiments, the population of polyploid seed or the subpopulation of genetically uniform polyploid seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe components wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the partial MiMe genotype comprises one or more non-MiMe alleles at the one or more MiMe loci of each of the first, second, and third MiMe components. In other variations, the partial MiMe genotype comprises two or more non-MiMe alleles at the one or more MiMe loci of each of the first, second, and third MiMe components.

In some embodiments, the population of polyploid seed or the subpopulation of genetically uniform polyploid seed has a complete MiMe genotype comprising MiMe alleles conferring decreased expression of one or more MiMe loci each of a first and second MiMe component, wherein the first Mime component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the population of polyploid seed or the subpopulation of genetically uniform polyploid seed having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes.

In other embodiments, the population of polyploid seed or the subpopulation of genetically uniform polyploid seed has a partial MiMe genotype comprising (a) one or more MiMe alleles conferring decreased expression of one or more MiMe loci of each of a first and second MiMe component; and (b) one or more non-MiMe alleles at the one or more MiMe loci of each of the first and second MiMe components wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the partial MiMe genotype comprises one or more non-MiMe alleles at the one or more MiMe loci of each of the first and second MiMe components. In other variations, the partial MiMe genotype comprises two or more non-MiMe alleles at the one or more MiMe loci of each of the first and second MiMe components.

In yet other embodiments, the population of polyploid seed or the subpopulation of genetically uniform polyploid seed has (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the third haplotype comprises a non-MiMe allele at the one or more MiMe loci of one or more of the first, second, and third MiMe components.

In still other embodiments, the population of polyploid seed or the subpopulation of genetically uniform polyploid seed has (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the third haplotype comprises a non-MiMe allele at the one or more MiMe loci of one or more of the first and second MiMe components.

In some embodiments, the population of polyploid seed or the subpopulation of genetically uniform polyploid seed has a partially-complemented MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the partially-complemented MiMe genotype comprises (a) only MiMe alleles at one or more MiMe loci of the first Mime component; (b) one or more MiMe alleles and one or more non-MiMe alleles at a first Mime locus of the second MiMe component, and one or more MiMe alleles and one or more non-MiMe alleles at a second MiMe locus of the second MiMe component; and (c) either (i) only MiMe alleles at one or more MiMe loci of the third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component. In some embodiments, the partially-complemented MiMe genotype comprises (a) only MiMe alleles at one or more MiMe loci of a component of sister chromatid cohesion during the first division of meiosis; (b) one or more MiMe alleles and one or more non-MiMe alleles at a first MiMe locus of a component of DNA double strand breakage during meiotic recombination, and one or more MiMe alleles and one or more non-MiMe alleles at a second MiMe locus of the component of DNA double strand breakage during meiotic recombination; and (c) either (i) only MiMe alleles at one or more MiMe loci of a component of progression through the second division of meiosis, or (ii) one or more MiMe alleles and one or more non-MiMe alleles at a first MiMe locus of a component of progression through the second division of meiosis, and one or more MiMe alleles and one or more non-MiMe alleles at a second Mime locus of the component of progression through the second division of meiosis. Exemplary MiMe loci of each of said MiMe components are extensively described herein below. In one embodiment, the partially-complemented MiMe genotype comprises (a) only MiMe alleles of REC8; (b) one or more MiMe alleles and one or more non-MiMe alleles of SPO11-1, and one or more MiMe alleles and one or more non-MiMe alleles of PAIR1; and (c) only MiMe alleles of OSD1.

In other embodiments, the population of polyploid seed or the subpopulation of genetically uniform polyploid seed has a partially-complemented MiMe genotype comprising MiMe alleles conferring decreased expression of one or more MiMe loci each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the population of polyploid seed or the subpopulation of genetically uniform polyploid seed having a partially-complemented MiMe genotype comprising MiMe alleles conferring decreased expression of MiMe loci of (2) a component of DNA double strand breakage during meiotic recombination and (4) a component of progression through the first division of meiosis exhibits neither a MiMe phenotype nor a wild-type meiosis phenotype in male germline cells and does not produce viable male gametes, and exhibits a wild-type meiosis phenotype in female germline cells and/or produces viable haploid female gametes. In some embodiments, the partially-complemented MiMe genotype comprises (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component. In certain embodiments, the partially-complemented MiMe genotype comprises (a) only MiMe alleles at one or more MiMe loci of a component of DNA double strand breakage during meiotic recombination; and (b) one or more MiMe alleles and one or more non-MiMe alleles at a first MiMe locus of a component of progression through the first division of meiosis, and one or more MiMe alleles and one or more non-MiMe alleles at a second MiMe locus of the component of progression through the first division of meiosis. In other embodiments, the partially-complemented MiMe genotype comprises (a) only MiMe alleles at one or more MiMe loci of a component of progression through the first division of meiosis; and (b) one or more MiMe alleles and one or more non-MiMe alleles at a first MiMe locus of a component of DNA double strand breakage during meiotic recombination and one or more MiMe alleles and one or more non-MiMe alleles at a second MiMe locus of the component of DNA double strand breakage during meiotic recombination. Exemplary MiMe loci of each of said MiMe components are extensively described below.

In some embodiments, the population of polyploid seed or the subpopulation of genetically uniform polyploid seed has a partially-complemented MiMe genotype comprising MiMe alleles conferring decreased expression of one or more MiMe loci of a first, second, third, and fourth MiMe component are selected from the group consisting of (1) a component of sister chromatid cohesion during the first division of meiosis, (2) a component of DNA double strand breakage during meiotic recombination, (3) a component of progression through the second division of meiosis, and (4) a component of progression through the first division of meiosis. In certain embodiments, the partially-complemented genotype comprises (a) only MiMe alleles at one or more MiMe loci of the first MiMe component, wherein the first Mime component is a component of DNA double strand breakage during meiotic recombination; (b) one or more MiMe alleles and one or more non-MiMe alleles at one or more MiMe loci of the second MiMe component; (c) one or more MiMe alleles and one or more non-MiMe alleles at one or more MiMe loci of the third MiMe component; (d) one or more MiMe alleles and one or more non-MiMe alleles at one or more MiMe loci of the fourth MiMe component. In some embodiments, the partially-complemented genotype comprises (a) only MiMe alleles at one or more MiMe loci of a component of DNA double strand breakage during meiotic recombination; (b) one or more MiMe alleles and one or more non-MiMe alleles at one or more MiMe loci of a component of sister chromatid cohesion during the first division of meiosis; (c) one or more MiMe alleles and one or more non-MiMe alleles at one or more MiMe loci of a component of progression through the second division of meiosis; (d) one or more MiMe alleles and one or more non-MiMe alleles at one or more MiMe loci of a component of progression through the first division of meiosis. Exemplary MiMe loci of each of said MiMe components are extensively described below. In one variation, the partially-complemented genotype comprises (a) only MiMe alleles at SPO11-1; (b) one or more MiMe alleles and one or more non-MiMe alleles at REC8; (c) one or more MiMe alleles and one or more non-MiMe alleles at OSD1; (d) one or more MiMe alleles and one or more non-MiMe alleles at PS1 or JASON.

In certain embodiments, the complete MiMe genotype, the partial MiMe genotype, or the partially-complemented MiMe genotype comprises one or more alleles that result in the production of unreduced (2n) gametes. The one or more alleles that result in the production of unreduced (2n) gametes can be a naturally occurring allele such as, for example, an os allele or a ps allele. os alleles are known in the art and described in, for example, J. E. Werner & S. J. Peloquin (Inheritance and Two Mechanisms of 2n Egg Fromation in 2× Potatoes, Journal of Heredity, Volume 81, Issue 5, September 1990, Pages 371-374) and J. E. Werner & S. J. Peloquin (Occurrence and mechanisms of 2n egg formation in 2× potato. Genome. 34(6): 975-982). ps alleles are known in the art and described in, for example, Mok, D. W. S. & Peloquin, S. J. (1975. Breeding value of 2n pollen (diplandroids) in tetraploid x diploid crosses in potatoes. Theor. Appl. Genet. 46: 307-314) and Watanabe K. (2015. Potato genetics, genomics, and applications. Breed Sci. 65(1):53-68).

In some embodiments, the population of polyploid seed or the subpopulation of genetically uniform polyploid seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid seed or the subpopulation of genetically uniform polyploid seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the population of polyploid seed is maize seed and the one or more OSD1 loci comprise OSD1-1, OSD1-2, and/or OSD1-3, and/or the one or more PAIR1 loci comprise PAIR1-1 and/or PAIR1-2. In other variations, the population of polyploid seed is soybean seed and the one or more OSD1 loci comprise OSD1-1 and/or OSD1-2.

In some embodiments, the population of polyploid seed or the subpopulation of genetically uniform polyploid seed has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In some variations, the population of polyploid seed is maize seed and the one or more OSD1 loci comprise OSD1-1, OSD1-2, and/or OSD1-3. In other variations, the population of polyploid seed is soybean seed and the one or more OSD1 loci comprise OSD1-1 and/or OSD1-2.

In some embodiments, the population of polyploid seed or the subpopulation of genetically uniform polyploid seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid seed or the subpopulation of genetically uniform polyploid seed has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid seed or the subpopulation of genetically uniform polyploid seed has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the population of polyploid seed is maize seed and the one or more PAIR1 loci comprise PAIR1-1 and/or PAIR1-2.

In some embodiments, the population of polyploid seed or the subpopulation of genetically uniform polyploid seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In some variations, the population of polyploid seed is soybean seed and the one or more TDM1 loci comprise TDM1-1 and/or TDM1-2.

In some embodiments, the population of polyploid seed or the subpopulation of genetically uniform polyploid seed has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some embodiments, the population of polyploid seed or the subpopulation of genetically uniform polyploid seed has a partial MiMe genotype comprising (i) at least one MiMe allele and at least one non-MiMe allele at one or more TDM1 loci, wherein each of the Mime alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the population of polyploid seed is soybean seed and the one or more TDM1 loci comprise TDM1-1 and/or TDM1-2. In some variations, the population of polyploid seed is maize seed and the one or more PAIR1 loci comprise PAIR1-1 and/or PAIR1-2.

In some embodiments, the population of polyploid seed or the subpopulation of genetically uniform polyploid seed has a complete MiMe genotype comprising (i) an os allele, wherein the subpopulation of genetically uniform polyploid seed is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) an os allele, wherein the subpopulation of genetically uniform polyploid seed is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid seed or the subpopulation of genetically uniform polyploid seed has a complete MiMe genotype comprising (i) a ps allele, wherein the subpopulation of genetically uniform polyploid seed is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1. In some embodiments, the population of polyploid seed or the subpopulation of genetically uniform polyploid seed has a partial MiMe genotype comprising (i) a ps allele, wherein the subpopulation of genetically uniform polyploid seed is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the population of polyploid seed or the subpopulation of genetically uniform polyploid seed has a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1. In some variations, the population of polyploid seed is maize seed and the one or more OSD1 loci comprise OSD1-1, OSD1-2, and/or OSD1-3, and/or the one or more PAIR1 loci comprise PAIR1-1 and/or PAIR1-2. In other variations, the population of polyploid seed is soybean seed and the one or more OSD1 loci comprise OSD1-1 and/or OSD1-2.

In some embodiments, the population of polyploid seed or the subpopulation of genetically uniform polyploid seed has a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the Mime alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1.

In some embodiments, the population of polyploid seed or the subpopulation of genetically uniform polyploid seed has a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In some variations, the population of polyploid seed is soybean seed and the one or more TDM1 loci comprise TDM1-1 and/or TDM1-2. In some variations, the population of polyploid seed is maize seed and the one or more PAIR1 loci comprise PAIR1-1 and/or PAIR1-2.

In some embodiments, the population of polyploid seed or the subpopulation of genetically uniform polyploid seed has a partially complemented MiMe genotype comprising (i) a ps allele, wherein the subpopulation of genetically uniform polyploid seed is heterozygous for the ps allele, (ii) an os allele, wherein the subpopulation of genetically uniform polyploid seed is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1.

In some embodiments, the population of polyploid seed or the subpopulation of genetically uniform polyploid seed has a partially complemented MiMe genotype comprising (i) an os allele, wherein the subpopulation of genetically uniform polyploid seed is heterozygous for the os allele, (ii) at least one Mime allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.

In some embodiments, the subpopulation of genetically uniform polyploid seed comprises one or more polynucleotide sequences selected from the group consisting of SEQ ID NOs: 448-466, 468-471, 474-491, and 493-496.

In some embodiments, the population of polyploid seed is from a maize plant, and the subpopulation of genetically uniform polyploid maize seed comprises one or more polynucleotide sequences selected from the group consisting of SEQ ID NOs: 448-451. In certain embodiments, the subpopulation of genetically uniform polyploid maize seed comprises ay a MiMe allele at one or more OSD1-2 loci, each independently comprising a polynucleotide sequence selected from the group consisting of SEQ ID NOs: 448 and 449; b) a MiMe allele at a REC8 locus comprising the polynucleotide sequence of SEQ ID NO: 450; and/or c) a MiMe allele at a SPO11-1 locus comprising the polynucleotide sequence of SEQ ID NO: 451. In some variations, each the one or more OSD1-2 loci, each of the one or more REC8 loci, and/or each of the one or more SPO11-1 loci are present on a different homologous chromosome.

In some embodiments, the population of polyploid seed is from a potato plant, and the subpopulation of genetically uniform polyploid potato seed comprises one or more polynucleotide sequences selected from the group consisting of SEQ ID NOs: 464-496. In certain embodiments, the subpopulation of genetically uniform polyploid potato seed comprises a MiMe allele at one or more SPO11-1 loci, each independently comprising a polynucleotide sequence selected from the group consisting of SEQ ID NOs: 464-466. In certain embodiments, the subpopulation of genetically uniform polyploid potato seed comprises a) a MiMe allele at one or more REC8l loci, each independently comprising a polynucleotide sequence selected from the group consisting of SEQ ID NOs: 468 and 469; and/or b) a MiMe allele at one or more SPO11-1 loci, each independently comprising a polynucleotide sequence selected from the group consisting of SEQ ID NOs: 470 and 471. In certain embodiments, the subpopulation of genetically uniform polyploid potato seed comprises a) a MiMe allele at one or more REC8 loci, each independently comprising a polynucleotide sequence selected from the group consisting of SEQ ID NOs: 474 and 475; and/or b) a MiMe allele at one or more SPO11-1 loci, each independently comprising a polynucleotide sequence selected from the group consisting of SEQ ID NOs: 476 and 477. In certain embodiments, the subpopulation of genetically uniform polyploid potato seed comprises a MiMe allele at one or more SPO11-1 loci, each independently comprising a polynucleotide sequence selected from the group consisting of SEQ ID NOs: 478 and 479. In certain embodiments, the subpopulation of genetically uniform polyploid potato seed comprises a) a MiMe allele at one or more CYCA1 loci, each independently comprising a polynucleotide sequence selected from the group consisting of SEQ ID NOs: 480 and 481; b) a MiMe allele at one or more REC8 loci, each independently comprising a polynucleotide sequence selected from the group consisting of SEQ ID NOs: 482 and 483; and/or c) a MiMe allele at one or more SPO11-1 loci, each independently comprising a polynucleotide sequence selected from the group consisting of SEQ ID NOs: 484 and 485. In certain embodiments, the subpopulation of genetically uniform polyploid potato seed comprises a) a MiMe allele at one or more CYCA1 loci, each independently comprising a polynucleotide sequence selected from the group consisting of SEQ ID NOs: 486 and 487; b) a MiMe allele at one or more REC8 loci, each independently comprising a polynucleotide sequence selected from the group consisting of SEQ ID NOs: 488 and 489; and/or c) a MiMe allele at one or more SPO11-1 loci, each independently comprising a polynucleotide sequence selected from the group consisting of SEQ ID NOs: 490 and 491. In certain embodiments, the subpopulation of genetically uniform polyploid potato seed comprises a) a MiMe allele at one or morn REC8 Loci, each independently comprising a polynucleotide sequence selected from the group consisting of SEQ ID NOs: 493 and 494; and/or c) a MiMe allele at one or more SPO11-1 loci, each independently comprising a polynucleotide sequence selected from the group consisting of SEQ ID NOs: 495 and 496. In some variations, each the one or more CYCA loci, the one or more REC8 loci, and/or the one or more SPO11-1 loci are present on a different homologous chromosome.

In some embodiments, the population of polyploid seed is from an Arabidopsis plant, and the subpopulation of genetically uniform polyploid Arabidopsis seed comprises one or more polynucleotide sequences selected from the group consisting of SEQ ID NOs: 452-463. In certain embodiments, the subpopulation of genetically uniform polyploid Arabidopsis seed comprises a) a MiMe allele at one or more OSD1 loci, each independently comprising a polynucleotide sequence selected from the group consisting of SEQ ID NOs: 452 and 453; b) a MiMe allele at one or more REC8 loci, each independently comprising a polynucleotide sequence selected from the group consisting of SEQ ID NOs: 454 and 455; and/or c) a MiMe allele at one or more SPO11-1 loci, each independently comprising a polynucleotide sequence selected from the group consisting of SEQ ID NOs: 456 and 457. In certain embodiments, the subpopulation of genetically uniform polyploid Arabidopsis seed comprises a) a MiMe allele at one or more OSD1 loci, each independently comprising a polynucleotide sequence selected from the group consisting of SEQ ID NOs: 458 and 459; b) a MiMe allele at one or more REC8 loci, each independently comprising a polynucleotide sequence selected from the group consisting of SEQ ID NOs: 460 and 461; and/or c) a Mime allele at one or more PAIR1 loci, each independently comprising a polynucleotide sequence selected from the group consisting of SEQ ID NOs: 462 and 463. In some variations, each the one or more OSD1 loci, the one or more REC8 loci, the one or more PAIR1 loci, and/or the one or more SPO11-1 loci are present on a different homologous chromosome.

In some embodiments, the population of polyploid seed comprises one or more genetic modifications resulting in decreased expression of one or more MiMe loci. In certain embodiments, the genetic modification resulting in decreased expression of a MiMe locus is positioned in the first 70%, the first 60%, the first 50%, the first 40%, the first 30%, the first 20%, or the first 10% of the nucleotides of the coding sequence of the MiMe locus following the start codon in the 3′ direction. In some embodiments, the genetic modification resulting in decreased expression of a MiMe locus is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction. In some embodiments, the genetic modification resulting in decreased expression of a MiMe locus is an insertion, a deletion, one or more nucleotide changes, or an inversion. In some variations, the insertion, the deletion, the one or more nucleotide changes, or the inversion eliminates expression (e.g., eliminates activity) of the MiMe locus. In certain embodiments, the insertion, the deletion, the one or more nucleotide changes, or the inversion is positioned in the first 70%, the first 60%, the first 50%, the first 40%, the first 30%, the first 20%, or the first 10% of the nucleotides of the coding sequence of the MiMe locus following the start codon in the 3′ direction. In certain embodiments, the insertion, the deletion, the one or more nucleotide changes, or the inversion is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction. In certain embodiments, the insertion, the deletion, the one or more nucleotide changes, or the inversion results in a premature stop codon present in the first 70%, the first 60%, the first 50%, the first 40%, the first 30%, the first 20%, or the first 10% of the nucleotides of the coding sequence of the MiMe locus following the start codon in the 3′ direction, thereby eliminating expression (e.g., activity) of the MiMe locus. In certain embodiments, the insertion, the deletion, the one or more nucleotide changes, or the inversion results in a premature stop codon present in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction, thereby eliminating expression (e.g., activity) of the MiMe locus.

In some embodiments, the one or more genetic modifications resulting in decreased expression of one or more MiMe loci comprise one or more sequences selected from the group consisting of SEQ ID NOs: 448-466, 468-471, 474-491, and 493-496.

### Components of Sister Chromatid Cohesion During the First Division of Meiosis

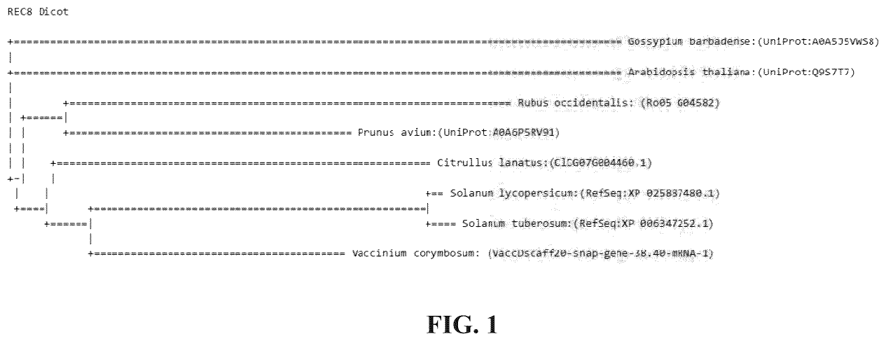

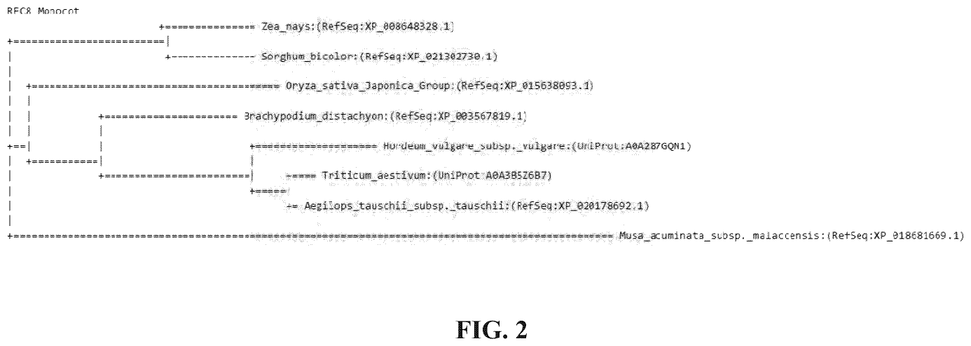

In some embodiments, the complete, partial, or partially-complemented MiMe genotype comprises one or more MiMe alleles conferring decreased expression of one or more MiMe loci of a component of sister chromatid cohesion during the first division of meiosis. In certain embodiments, the one or more MiMe loci of the component of sister chromatid cohesion during the first division of meiosis comprise REC8, SWITCH1/DYAD, or a combination thereof. One of skill in the art will understand that MiMe loci that are components of sister chromatid cohesion during the first division of meiosis are not limited to REC8 and SWITCH1/DYAD, and may include any loci encoding gene products required for sister chromatid cohesion during the first division of meiosis. By way of example only, a gene product of the component of sister chromatid cohesion during the first division of meiosis is exemplified by a REC8 protein and specifically by the REC8 protein sequences, sequence alignments, and percent identities described in “MiMe Gene Product Sequences” below. Representative REC8 protein sequences from dicotyledonous plants (SEQ ID NOs: 1-9 and 88) are provided in the sequence listing as outlined in Table 9, including eight native sequences and a consensus sequence identified by multiple sequence alignment of the eight native sequences (Sequence Alignment 1). Table 1 shows a matrix of percent identities of the REC8 protein sequences from dicotyledonous plants, and a phylogenetic tree showing the relationship between the sequences is shown in FIG. 1. Representative REC8 protein sequences from monocotyledonous plants (SEQ ID NOs: 10-18) are provided in the sequence listing as outlined in Table 9, including eight native sequences and a consensus sequence identified by multiple sequence alignment of the eight native sequences (Sequence Alignment 2). Table 2 shows a matrix of percent identities of the REC8 protein sequences from monocotyledonous plants, and a phylogenetic tree showing the relationship between the sequences is shown in FIG. 2. The gene products of MiMe loci of the component of sister chromatid cohesion during the first division of meiosis include REC8 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the REC8 proteins of SEQ ID NOs: 1-18 and 88.

There is an abundance of known REC8 gene products from both dicotyledonous plants (dicots) and monocotyledonous plants (monocots) including all of the following, which sequences are hereby incorporated by reference in their form as of the effective filing date. Exemplary REC8 gene products from dicots include those of, by way of example, Abrus precatorius (XP 027368514.1), Acer yangbiense (A0A5C7GZ90), Arabidopsis lyrata subsp. lyrata (XP 002871163.1 and XP 020875944.1), Arabidopsis thaliana (A0A178UHG0, A0A654FZ23, and Q9S7T7), Arabis nemorensis (A0A565CI70), Arachis duranensis (XP 015973751.1, XP 015973752.1, XP 020983026.1, and XP 020983028.1), Arachis hypogaea (A0A444YDK7 and A0A445C522), Beta vulgaris subsp. vulgaris (XP 010687480.1), Brassica campestris (A0A397XP65), Brassica napus (XP 013741480.1, and XP 022566969.1), Brassica oleracea var. oleracea (XP 013610020.1, XP 013610021.1, XP 013610022.1, and XP 013610024.1), Brassica rapa (XP 033137771.1), Brassica rapa subsp. pekinensis (M4CY18), Cajanus cajan (XP 020204411.1 and A0A151U182), Camelina sativa (XP 010423466.1 and XP 010452452.1), Camellia sinensis (XP 028104672.1), Camellia sinensis var. sinensis (A0A4S4ESL6), Cannabis sativa (XP 030485337.1 and XP 030490395.1), Capsicum annuum (A0A2G2ZDH7), Capsicum baccatum (A0A2G2WNK5), Carica papaya (XP 021904051.1), Carpinus fangiana (A0A5N6QBL7), Cephalotus follicularis (A0A1Q3D6L7), Chenopodium quinoa (XP 021769482.1), Cicer arietinum (A0A3Q7Y8G8), Citrullus lanatus (MAKER: Cla97C04G068650.1, Cla97C07G132920.1, Cla97C11G213870.1, C1CG07G004460.1, and C1CG07G004460.1), Citrus clementina (XP_024041762.1 and V4V9Y1), Citrus sinensis (A0A067FZJ6), Citrus unshiu (A0A2H5NJX2 and A0A2H5NK81), Coffea arabica (XP 027104216.1, XP 027104218.1, A0A6P6VMF6, A0A6P6VMG1, A0A6P6VNE3, and A0A6P6W6N8), Coffea canephora (A0A068V055), Coffea eugenioides (XP 027161382.1), Corchorus capsularis (A0A1R3FW92), Corchorus olitorius (A0A1R3I015), Cucumis melo (A0A1S3BZ41), Cucumis melo var. makuwa (A0A5A7SX09 and A0A5D3E364), Cucumis sativus (XP 031742082.1), Cucurbita maxima (XP 022996758.1), Cucurbita moschata (XP 022941876.1), Cucurbita pepo subsp. pepo (XP 023543038.1 and XP 023552287.1), Cuscuta campestris (A0A484LMF6), Cynara cardunculus var. scolymus (XP 024992312.1), Daucus carota subsp. sativus (XP 017239103.1), Dorcoceras hygrometricum (A0A2Z7BXH5), Durio zibethinus (XP_022743895.1), Erythranthe guttata (XP_012858265.1 and A0A022PW80), Eucalyptus grandis (XP 010068917.1 and A0A059B1I30), Eutrema salsugineum (XP 006400164.1, XP 024011536.1, and V4LKV8), Fragaria vesca subsp. vesca (XP 011464664.1), Glycine max (K7M545), Glycine soja (A0A445LUE6), Gossypium arboreum (XP_052882704.1), Gossypium barbadense (A0A5J5RSH8 and A0A5J5VWS8), Gossypium darwinii (A0A5D2CX24), Gossypium mustelinum (A0A5D2VBC9), Gossypium raimondii (XP 012466880.1 and A0A0D2TKD5), Gossypium tomentosum (A0A5D2QPB5), Helianthus annuus (XP 021977217.1 and XP 021977218.1), Hevea brasiliensis (XP 021640351.1), Hibiscus syriacus (A0A6A3BZC0 and A0A6A3CV01), Ipomoea triloba (XP 031095284.1), Jatropha curcas (XP 020540958.1 and A0A067LKA2), Juglans regia (XP 018821798.1), Lactuca saligna (A0A6S7NZN4), Lactuca sativa (XP 023771132.1), Lupinus albus (A0A6A5NW87), Lupinus angustifolius (XP 019461100.1), Manihot esculenta (XP 021626581.1), Medicago truncatula (XP 003616515.2 and XP 024638994.1), Microthlaspi erraticum (A0A6D2ITW2), Mikania micrantha (A0A5N6LUS6 and A0A5N6LWZ3), Momordica charantia (XP 022146768.1), Morus notabilis (XP 024021421.1), Nicotiana attenuata (XP 019262849.1), Nicotiana sylvestris (A0A1U7WT01), Nicotiana tabacum (XP 016494893.1), Nicotiana tomentosiformis (XP 009617668.1), Parasponia andersonii (A0A2P5BRF9), Phaseolus vulgaris (XP 007141742.1, XP 007145188.1, XP 007145189.1, and XP 007145191.1), Populus alba (XP 034923854.1 and A0A4U5QBL2), Populus trichocarpa (XP 002312177.2 and A0A2K1ZDB6), Prunus armeniaca (A0A6J5U4U0 and A0A6J5WI99), Prunus avium (A0A6P5RL70, A0A6P5RV91, and A0A6P5RWY0), Prunus dulcis (XP 034197561.1, A0A5E4ESH1, A0A5E4EV88, and A0A5E4GL51), Prunus mume (XP 008233800.1), Prunus persica (XP 020413029.1, A0A251QLJ9, and M5X441), Prunus yedoensis var. nudiflora (A0A314XY06), Punica granatum (XP 031379523.1), Pyrus ussuriensis x Pyrus communis (A0A5N5HRH5), Quercus lobata (XP 030932817.1), Quercus suber (XP 023928884.1), Raphanus sativus (XP_018444222.1 and A0A6J0K9W8), Rhodamnia argentea (XP 030534655.1), Ricinus communis (XP 015575710.2 and B9S452), Rosa chinensis (XP 024172133.1, XP 024174158.1, and XP 024175438.1), Rubus occidentalis (MAKER: Ro05_G04582 and Ro05_G04582), Salix viminalis (A0A6N2M747), Salvia splendens (A0A4D8YMH3 and A0A4D8YVU8), Solanum chilense (A0A6N2AMP7), Solanum lycopersicum (XP 025887480.1 and XP 025887481.1), Solanum pennellii (XP 027773809.1), Solanum tuberosum (XP 006347252.1), Spinacia oleracea (XP 021841553.1 and A0A0K9QU15), Striga asiatica (A0A5A7R0R4), Syzygium oleosum (XP 030451237.1), Tarenaya hassleriana (XP 010543708.1, and XP 010543709.1), Theobroma cacao (XP 007008892.2), Trema orientale (A0A2P5BAB6), Trifolium subterraneum (A0A2Z6LP94 and A0A2Z6NAY5), Vaccinium corymbosum (MAKER: VaccDscaff20-snap-gene-38.40-mRNA-1, VaccDscaff20-snap-gene-38.40-mRNA-1, and VaccDscaff28-processed-gene-318.6-mRNA-1), Vigna angularis (XP 017428323.1), Vigna radiata var. radiata (XP 022637684.1 and A0A1S3UAC8), Vigna unguiculata (XP 027939010.1 and A0A4D6MIK7), Vitis riparia (XP 034703755.1 and XP 034703756.1), Vitis vinifera (XP 002273379.3, XP 010658235.1, XP 010658236.1, and XP 010658238.1), and Ziziphus jujuba (XP 024927899.1 and A0A6P3ZPX3). Exemplary REC8 gene products from monocots include those of, by way of example, Aegilops tauschii subsp. strangulata (A0A453A0K3), Aegilops tauschii subsp. tauschii (XP 020178692.1), Ananas comosus (XP 020086789.1), Asparagus officinalis (XP 020274504.1 and A0A5P1ED15), Brachypodium distachyon (XP 003567819.1, A0A0Q3JE03, and A0A2K2D8P1), Dendrobium catenatum (A0A2I0VMA3), Dichanthelium oligosanthes (A0A1E5UXI5), Elaeis guineensis (XP 010921544.1), Hordeum vulgare subsp. vulgare (A0A287FWE3, A0A287GQN1, and F2DF29), Leersia perrieri (A0A0D9WK98, A0A0D9WK99, A0A0D9WKA0, A0A0D9WKA1, and A0A0D9WKA2), Musa acuminata subsp. malaccensis (XP 018681669.1 and M0T2D3), Musa balbisiana (A0A4S8JZT1), Oryza barthii (A0A0D3GB92), Oryza brachyantha (XP 015692957.1), Oryza glaberrima (I1PYA2), Oryza glumipatula (A0A0E0A392), Oryza meridionalis (A0A0E0DUV5, A0A0E0DUV6, A0A0E0DUV7, and A0A0E0DUV9), Oryza meyeriana var. granulata (A0A6G1EFH8, A0A6G1EFK1, A0A6G1EFN6, A0A6G1EFP2, and A0A6G1EG47), Oryza nivara (A0A0E0HIZ6 and A0A0E0HIZ7), Oryza punctata (A0A0E0L649, A0A0E0L650, and A0A0E0L651), Oryza rufipogon (A0A0E0PRQ3, A0A0E0PRQ4, and A0A0E0PRQ5), Oryza sativa Japonica Group (XP 025881258.1, XP 025881259.1, and XP 015638093.1), Oryza sativa subsp. indica (B8AX57), Oryza sativa subsp. japonica (B9FLS4), Panicum hallii (XP 025808802.1 and A0A2T8KIF0), Panicum hallii var. hallii (A0A2T7E9Y6), Panicum miliaceum (A0A3L6RE55, A0A3L6SVY0, and A0A3L6T3B8), Phalaenopsis equestris (XP 020572128.1), Phoenix dactylifera (XP 008794300.1), Setaria italica (XP 022680769.1, A0A368QEG1, A0A368QF75, and K3Z532), Setaria viridis (XP 034584869.1, XP 034584871.1, XP 034584872.1, XP 034584873.1, and XP 034584874.1), Sorghum bicolor (XP 021302726.1, XP 021302727.1, XP 021302728.1, and XP 021302730.1), Triticum aestivum (A0A385JG01, A0A3B5Z4Y3, and A0A3B5Z6B7), Triticum turgidum (A0A385JG02 and A0A385JG05), Triticum turgidum subsp. durum (A0A446JDS8 and A0A446KDI4), Triticum urartu (M8APB3), Zea mays (NP 001105829.1, XP 008648327.1, XP 008648329.1, and XP 008648328.1), and Zostera marina (A0A0K9PVD4).

A gene product of the component of sister chromatid cohesion during the first division of meiosis is also exemplified by a SWITCH1 protein. There is an abundance of known SWITCH1 gene products from both dicotyledonous plants (dicots) and monocotyledonous plants (monocots) including all of the following, which sequences are hereby incorporated by reference in their form as of the effective filing date. Exemplary SWITCH1 gene products from dicots include those of, by way of example, Rubus occidentalis (MAKER: Ro01_G30226 and Ro07_G17281), Vaccinium corymbosum (MAKER: VaccDscaff29-snap-gene-89.34-mRNA-1, VaccDscaff33-processed-gene-249.1-mRNA-1 and VaccDscaff26-snap-gene-88.35-mRNA-1), Arabidopsis thaliana (NP_568757.2, NP_001332209.1 and NP_001031931.2), Arabidopsis lyrata (XP 020871971.1, XP 020876253.1 and D7MQZ4), Camelina sativa (XP 010482465.2, XP 019087921.1 and XP 010443032.1), Brassica oleracea (XP 013618744.1, XP 013618745.1 and XP 013606353.1), Raphanus sativus (XP 018454604.1, XP 018454606.1 and XP 018454605.1), Brassica napus (XP 013708231.1, XP 022575752.1 and XP 013661048.1), Brassica rapa (XP 033140888.1, XP 033140887.1 and XP 033140886.1), Eutrema salsugineum (XP 006401988.2, XP 024007513.1 and V4LLU7), Tarenaya hassleriana (XP 010536546.1 and XP 010521524.1), Theobroma cacao (XP 007038325.2, A0A061FZY7 and A0A061DS77), Manihot esculenta (XP 021592051.1, XP 021631569.1 and XP 021631143.1), Gossypium hirsutum (XP 016694472.1, XP 016747222.1 and A0A1U8JW21), Gossypium arboreum (XP 017633866.1, A0A6P4NXR3 and A0A0B0PTF4), Gossypium raimondii (XP 012490990.1, A0A0D2QKU7 and A0A0D2P486), Hevea brasiliensis (XP 021677124.1, XP 021686979.1 and XP 021685142.1), Carica papaya (XP 021905530.1), Juglans regia (XP 018815830.2, XP 018833959.1 and XP 018809099.1), Ziziphus jujuba (XP 024927739.1 and A0A6P6G0U9), Cucurbita maxima (XP 022969779.1 and A0A6J1I3M6), Quercus lobata (XP 030936886.1), Ricinus communis (XP 015578072.1, B9R9W1 and B9RW86), Abrus precatorius (XP 027356801.1, XP 027367066.1 and XP 027342753.1), Cucurbita moschata (XP 022929009.1 and A0A6J1EMH8), Cucumis sativus (XP 031741226.1, XP 011653872.1 and A0A0A0KM54), Momordica charantia (XP 022157545.1, A0A6J1DTE0 and A0A6J1DYK5), Populus trichocarpa (XP 002303485.3, XP 006368241.2 and XP 024455546.1), Cucurbita pepo (XP 023520530.1), Cucumis melo (XP 008437502.2, XP 008449870.1 and A0A1S3AU69), Quercus suber (XP 023877008.1 and XP 023901279.1), Cicer arietinum (XP 027192472.1, XP 004498031.1 and XP 004503018.1), Populus euphratica (XP 011027968.1, XP 011027967.1 and XP 011018358.1), Populus alba (XP 034888212.1, XP 034900951.1 and XP 034933293.1), Jatropha curcas (XP 020540831.1, A0A067JI11 and A0A067JN58), Glycine soja (XP 028201918.1, XP 028247254.1 and A0A445GWL2), Glycine max (XP 014623227.1, XP 006586432.1 and A0A0R0GBS0), Rhodamnia argentea (XP 030533147.1, XP 030551396.1 and XP 030551395.1), Eucalyptus grandis (XP 018723647.1, XP 010027481.1 and A0A059DKD1), Syzygium oleosum (XP 030469030.1 and XP 030462765.1), Vigna unguiculata (XP 027910422.1, XP 027922193.1 and XP 027913349.1), Cajanus cajan (XP 029128893.1, XP 020233511.1 and XP 020233515.1), Prunus persica (XP 020413041.1, XP 007212097.1 and A0A251PSK8), Durio zibethinus (XP 022756155.1, XP 022774802.1 and XP 022774804.1), Vigna angularis (XP 017431855.1, XP 017422412.1 and XP 017413329.1), Punica granatum (XP 031394957.1, XP 031400758.1 and XP 031400760.1), Phaseolus vulgaris (XP 007133471.1, XP 007137842.1 and V7AJM3), Prunus mume (XP 008234499.1, XP 008234124.1 and XP 008227717.1), Arachis hypogaea (XP 025694505.1, XP 029150597.1 and A0A445EV05), Medicago truncatula (XP 024627402.1, XP 024626030.1 and XP 024633446.1), Citrus sinensis (XP 024948131.1, XP 024948129.1 and XP 006466206.1), Arachis duranensis (XP 015953885.1, A0A6P4CM13 and A0A6P4BH29), Vitis vinifera (XP 019073646.1, XP 019074196.1 and A5B3X1), Malus domestica (XP 008391805.2, XP 008376397.2 and XP 017178673.2), Pyrus x (XP 018506973.1, XP 018499351.1 and XP 009372920.2), Citrus clementina (XP 024045098.1, XP 024044142.1 and XP 006426415.1), Rosa chinensis (XP 024182740.1, XP 024182732.1 and XP 024182747.1), Prunus dulcis (XP 034203721.1, A0A5E4FL50 and A0A4Y1QZP3), Fragaria vesca (XP 011466774.1, XP 004307793.1 and XP 004288640.1), Daucus carota (XP 017239184.1, XP 017239183.1 and XP 017239182.1), Vitis riparia (XP 034689547.1 and XP 034689546.1), Solanum lycopersicum (XP 019068267.2, XP 025885737.1 and A0A3Q7FLA0), Pistacia vera (XP 031266939.1, XP 031273233.1 and XP 031280233.1), Solanum pennellii (XP 015068878.2), Prunus avium (XP 021823384.1, XP 021806123.1 and XP 021806128.1), Nicotiana sylvestris (XP 009768556.1, XP 009768557.1 and A0A1U7VUD6), Nicotiana tomentosiformis (XP 033513896.1), Nicotiana tabacum (XP 016465105.1, XP 016502171.1 and A0A1S3ZKP1), Ipomoea triloba (XP 031094295.1), Olea europaea (XP 022862850.1), Capsicum annuum (XP 016567025.1, A0A2G2ZYE1 and A0A1U8G7A5), Nicotiana attenuata (XP 019226689.1 and A0A314KRA7), Chenopodium quinoa (XP 021743505.1, XP 021759608.1 and XP 021743506.1), Lactuca sativa (XP 023755621.1, XP 023760692.1 and A0A2J6LWN4), Cynara cardunculus (XP 024990591.1 and A0A103Y8N7), Helianthus annuus (XP 022037915.1, XP 022037914.1 and XP 022012237.1), Morus notabilis (XP 024027224.1, W9RZ46 and W9RCB6), Spinacia oleracea (XP 021843368.1, XP 021854785.1 and A0A0K9R736), Beta vulgaris (XP 010674297.1, XP 010671506.1 and XP 010671497.1), Camellia sinensis (XP 028061496.1), Erythranthe guttata (XP 012858696.1 and A0A022Q107), Coffea eugenioides (XP 027154930.1 and XP 027160597.1), Coffea arabica (XP 027101112.1, XP 027110800.1 and A0A6P6VGQ6), Sesamum indicum (XP 020549158.1 and XP 020549178.1), Ipomoea nil (XP 019182952.1), Herrania umbratica (XP 021301021.1 and A0A6J1BNI3), Lupinus angustifolius (XP 019429765.1, XP 019415494.1 and XP 019414588.1), Vigna radiata (XP 014490808.1, XP 022633259.1 and XP 022640106.1), Microthlaspi erraticum (A0A6D2L0P7, A0A6D2JSG4 and A0A6D2KBK2), Arabis nemorensis (A0A565CPI1 and A0A565CV38), Brassica oleracea (A0A3P6DF14, A0A3P6F4R8 and A0A3P6E718), Brassica campestris (A0A3P6AIY7, A0A398ACV6 and A0A3P6DDU6), Brassica rapa (M4E197, M4DFC3 and M4CZQ1), Gossypium darwinii (A0A5D2FSA3, A0A5D2BXJ6 and A0A5D2AGW1), Gossypium barbadense (A0A5J5UZJ1, A0A5J5QMK9 and A0A2P5WB96), Gossypium tomentosum (A0A5D2PNI7, A0A5D2K2W4 and A0A5D2N6G2), Cephalotus follicularis (A0A1Q3C6L4), Hibiscus syriacus (A0A6A3B5U1, A0A6A2WQU3 and A0A6A2ZQT7), Morella rubra (A0A6A1VDS9 and A0A6A1UM94), Fagus sylvatica (A0A2N9HYS6 and A0A2N9IBN5), Corchorus capsularis (A0A1R3JJA7 and A0A1R3J6I7), Salix viminalis (A0A6N2JYJ1, A0A6N2KQX9 and A0A6N2NKC1), Populus tomentosa (A0A172CPQ9 and A0A172CCI6), Cucumis melo (A0A5A7TGY9, A0A5A7T1T6 and A0A5D3DVN3), Acer yangbiense (A0A5C7IN15 and A0A5C7HU16), Carpinus fangiana (A0A5N6R193 and A0A5N6RX15), Gossypium mustelinum (A0A5D2YGB2, A0A5D2U453 and A0A5D2X2K2), Salix brachista (A0A5N5N9V5, A0A5N5P3K8 and A0A5N5IXR5), Phaseolus angularis (A0A0L9V480, A0A0L9UDD9 and A0A0L9TJ70), Vigna angularis (A0A0S3SA92, A0A0S3REI6 and A0A0S3QXZ6), Lupinus albus (A0A6A4P1H2, A0A6A5NJG1 and A0A6A4NL28), Trifolium subterraneum (A0A2Z6P723, A0A2Z6NHN4 and A0A2Z6P3M8), Gossypium australe (A0A5B6UK08 and A0A5B6W373), Camellia sinensis (A0A4S4EMR0), Malus baccata (A0A540MJP9, A0A540MK37 and A0A540KDD3), Citrus unshiu (A0A2H5QD63, A0A2H5QDA2 and A0A2H5QD51), Pyrus ussuriensis (A0A5N5F8Z7 and A0A5N5GTK0), Actinidia chinensis (A0A2R6PLV5 and A0A2R6RT36), Solanum tuberosum (M1BMI9), Brassica cretica (A0A3N6TEZ4 and A0A3N6RFR2), Solanum chilense (A0A6N2BEC5), Cuscuta campestris (A0A484LML2), Corchorus olitorius (A0A1R3JTT6 and A0A1R3GSW6), Nyssa sinensis (A0A5J5BAZ9), Trifolium pratense (A0A2K3PDT7, A0A2K3L8X7 and A0A2K3MMJ0), Capsicum chinense (A0A2G3CVL6), Cuscuta australis (A0A328E088), Dorcoceras hygrometricum (A0A2Z7BCL3), Lactuca saligna (A0A6S7P0V1), Salvia splendens (A0A4D9C1P9, A0A4D9AGX1 and A0A4D9AK68), Mikania micrantha (A0A5N6LLV9), Prunus armeniaca (A0A6J5U7Z8, A0A6J5X5F7 and A0A6J5US78), Capsicum baccatum (A0A2G2X4T2), Trema orientale (A0A2P5F3Q1, A0A2P5EPH7 and A0A2P5EEN0), Parasponia andersonii (A0A2P5BQL6, A0A2P5BHP5 and A0A2P5AGC5), Striga asiatica (A0A5A7PQN9), Coffea canephora (A0A068U9Y0 and A0A068V0I1), Prunus yedoensis (A0A314Y7T9, A0A314YIJ8 and A0A314YG31), Trifolium medium (A0A392ME05), Populus davidiana (A0A6M2ETK8), Citrullus lanatus (MAKER: Cla97C01G001140.1, Cla97C04G070110.1 and Cla97C04G070180.1). Exemplary SWITCH1 gene products from monocots include those of, by way of example, Oryza sativa (XP 015631380.1, XP 025877854.1 and XP 025877853.1), Brachypodium distachyon (XP 003561753.1, XP 024314238.1 and XP 014753959.1), Zea mays (NP_001139538.1, XP 008662288.1 and C0RWW9), Aegilops tauschi (XP 020181895.1), Sorghum bicolor (XP 021320130.1, XP 021301584.1 and A0A1B6QJ31), Panicum hallii (XP 025793705.1, XP 025793703.1 and XP 025793702.1), Setaria viridis (XP 034592833.1, XP 034586810.1 and A0A4U6T5S8), Setaria italica (XP 004967854.1, XP 022685391.1 and XP 012700195.2), Oryza brachyantha (XP 015689996.1, J3LR73 and J3NF51), Musa acuminata (XP 018682921.1, M0TD15 and M0S3R3), Elaeis guineensis (XP 029117261.1), Asparagus officinalis (XP 020266624.1, XP 020269116.1 and A0A5P1EU37), Dendrobium catenatum (XP 020691672.1, XP 020691673.1 and A0A2I0X2G5), Phalaenopsis equestris (XP 020583684.1), Phoenix dactylifera (XP 026656658.1 and A0A3Q0HPM4), Ananas comosus (XP 020105611.1 and A0A6P5GAF8), Oryza sativa (Q53KW9, B9FA25 and A0A0P0W0U5), Oryza rufipogon (A0A0E0NYX7, A0A0E0NYX8 and A0A0E0NYX9), Oryza sativa (B8ANI7, A2ZMW8 and B8BN37), Oryza glumipatula (A0A0D9ZB48, A0A0D9ZB47 and A0A0D9ZB49), Oryza glaberrima (I1PI20), Oryza nivara (A0A0E0GR36 and A0A0E0JCM0), Oryza barthi (A0A0D3FLV6 and A0A0D3HWX4), Oryza punctata (A0A0E0KGW8 and A0A0E0MPR3), Oryza meyeriana (A0A6G1EG02 and A0A6G1DFI5), Leersia perrieri (A0A0D9VWY8 and A0A0D9Y1K9), Eragrostis curvula (A0A5J9WCH8 and A0A5J9WNU9), Dichanthelium oligosanthes (A0A1E5WBQ9), Triticum aestivum (A0A3B6LSJ8, A0A3B6MYN0 and A0A3B6LSL7), Triticum turgidum (A0A446UKP0, A0A446UKL8 and A0A446TDN8), Panicum miliaceum (A0A3L6S8L6, A0A3L6TLV5 and A0A3L6QS82), Panicum hallii (A0A2T7C335, A0A2T7C3D6 and A0A2T7EJF0), Hordeum vulgare (A0A287S8Y7, A0A287S8R7 and A0A287S8R0), Aegilops tauschi (M8BFT2), Aegilops tauschii (A0A453LPB1, A0A453LPY2 and A0A453LQ13), Oryza meridionalis (A0A0E0FE50), Ananas comosus (A0A6V7P0R7), Musa balbisiana (A0A4S8IUI5 and A0A4S8JSG3), Zostera marina (A0A0K9PA69), Triticum urartu (M8A7F4).

### Components of DNA Double Strand Breakage During Meiotic Recombination

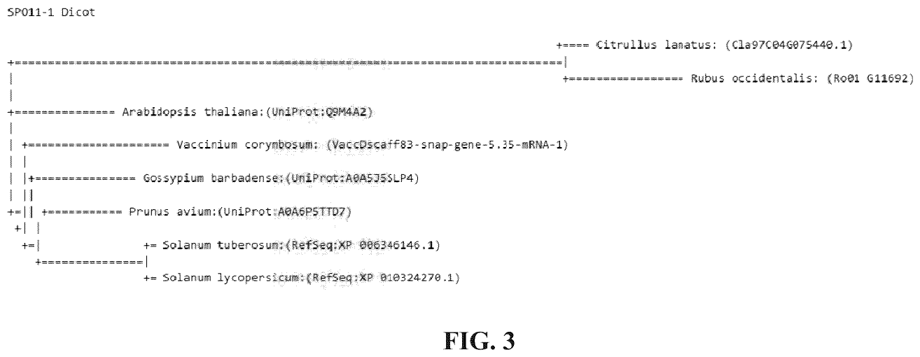

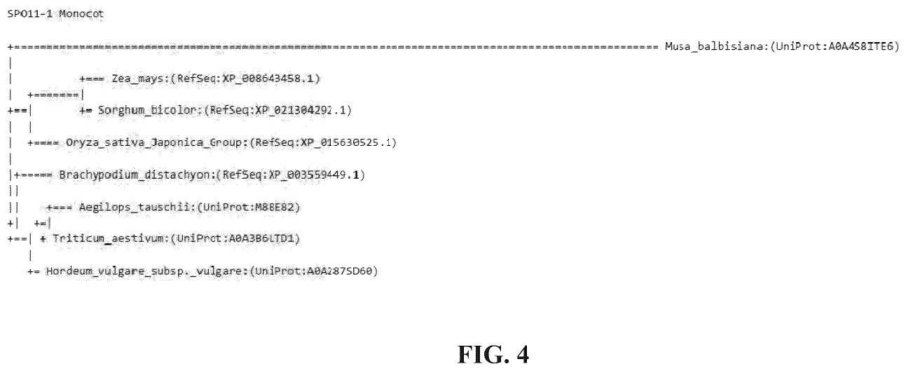

In some embodiments, the complete, partial, or partially-complemented MiMe genotype comprises one or more MiMe alleles conferring decreased expression of one or more MiMe loci of a component of DNA double strand breakage during meiotic recombination. In certain embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. One of skill in the art will understand that MiMe loci of the component of DNA double strand breakage during meiotic recombination are not limited to PAIR1, SPO11-1, SPO11-2, PRD1, PRD2, DFO, MTOPVIB, DSY1, SY1, SY2, SY3, and SY4 and may include any loci encoding gene products required for DNA double strand breakage during meiotic recombination. By way of example only, a gene product of the component of DNA double strand breakage during meiotic recombination is exemplified by a SPO11-1 protein and specifically by the SPO11-1 protein sequences, sequence alignments, and percent identities described in “MiMe Gene Product Sequences” below. Representative SPO11-1 protein sequences from dicotyledonous plants (SEQ ID NOs: 19-27 and 89) are provided in the sequence listing as outlined in Table 9, including eight native sequences and a consensus sequence identified by multiple sequence alignment of the eight native sequences (Sequence Alignment 3). Table 3 shows a matrix of percent identities of the SPO11-1 protein sequences from dicotyledonous plants, and a phylogenetic tree showing the relationship between the sequences is shown in FIG. 3. Representative SPO11-1 protein sequences from monocotyledonous plants (SEQ ID NOs: 28-36) are provided in the sequence listing as outlined in Table 9, including eight native sequences and a consensus sequence identified by multiple sequence alignment of the eight native sequences (Sequence Alignment 4). Table 4 shows a matrix of percent identities of the SPO11-1 protein sequences from monocotyledonous plants, and a phylogenetic tree showing the relationship between the sequences is shown in FIG. 4. The gene products of MiMe loci of the component of DNA double strand breakage during meiotic recombination include SPO11-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the SPO11-1 proteins of SEQ ID NOs: 19-36 and 89.

There is an abundance of known SPO11-1 gene products from both dicotyledonous plants (dicots) and monocotyledonous plants (monocots) including all of the following, which sequences are hereby incorporated by reference in their form as of the effective filing date. Exemplary SPO11-1 gene products from dicots include those of, by way of example, Abrus precatorius (XP 027329814.1), Acer yangbiense (A0A5C7HSF7 and A0A5C7IFY0), Actinidia chinensis var. chinensis (A0A2R6PR69), Arabidopsis lyrata subsp. lyrata (XP 002884949.1 and XP 020887397.1), Arabidopsis thaliana (NP 187923.1, A0A654FGW4, and Q9M4A2), Arabis alpina (A0A087G4D1), Arabis nemorensis (A0A565B723 and A0A565CHC5), Arachis duranensis (XP 015972943.1 and XP 020982698.1), Arachis hypogaea (XP 025608386.1, XP 025608387.1, and A0A444YFJ8), Beta vulgaris subsp. vulgaris (XP 010675908.1, XP 019104718.1, and A0A0J8B7P6), Brassica campestris (A0A024AGF2 and A0A398ASD7), Brassica cretica (A0A3N6Q3Z7 and A0A3N6QES9), Brassica napus (XP 013673221.1 and A0A078GGC9), Brassica oleracea var. oleracea (A0A0D3AE20), Brassica rapa subsp. pekinensis (M4CZB7 and M4FE53), Cajanus cajan (XP 020225124.1, XP 020225137.1, XP 020225143.1, and XP 029124728.1), Camelina sativa (XP 010465121.1), Camellia sinensis (XP 028114497.1, XP 028114498.1, XP 028114499.1, and XP 028114501.1), Camellia sinensis var. sinensis (A0A4S4DAX7), Cannabis sativa (XP 030509960.1), Capsicum annuum (XP 016542465.1, XP 016545530.1, A0A1U8ENS1, A0A1U8H6X1, A0A2G2XTT2, and A0A2G2Z5U1), Capsicum baccatum (A0A2G2VZF9), Capsicum chinense (A0A2G3BJL6), Carica papaya (XP 021905935.1 and XP 021905936.1), Carpinus fangiana (A0A5N6RMK4 and A0A5N6RSR0), Cephalotus follicularis (A0A1Q3CVM9), Chenopodium quinoa (XP 021740678.1 and XP 021771341.1), Cicer arietinum (XP 004491659.1 and A0A1S2Y0N1), Citrullus lanatus (Cla97C04G075440.1, Cla97C01G002360.1, Cla97C04G075440.1, C1CG01G002330.1, and ClCG09G021210.1), Citrus clementina (XP 024033994.1 and XP 024033995.1), Citrus sinensis (XP 006474908.2 and XP 015384601.2), Citrus unshiu (A0A2H5P5P7), Coffea arabica (A0A6P6SBT4, A0A6P6SCZ1, and A0A6P6ST96), Coffea canephora (A0A068UEN2 and A0A068UZB0), Coffea eugenioides (XP 027172596.1), Corchorus capsularis (A0A1R3FW98, A0A1R3HQV9, and A0A1R3IMB6), Corchorus olitorius (A0A1R3HTH1 and A0A1R3KL47), Cucumis melo (XP 008462990.1 and XP 016902962.1), Cucumis melo var. makuwa (A0A5A7TWM5), Cucumis sativus (XP 031744433.1, XP 031744436.1, XP 031744437.1, and XP 031745991.1), Cucurbita maxima (XP 022976331.1 and A0A6J1IM23), Cucurbita moschata (XP 022937001.1 and A0A6J1GGP2), Cucurbita pepo subsp. pepo (XP 023536168.1), Cuscuta australis (A0A328D1R5), Cuscuta campestris (A0A484N6Q1), Cynara cardunculus var. scolymus (XP 024971001.1 and A0A103XGW7), Daucus carota subsp. sativus (XP 017251399.1, XP 017251400.1, and XP 017251401.1), Dorcoceras hygrometricum (A0A2Z7CYH2), Durio zibethinus (XP 022753907.1 and A0A6P5YR36), Erythranthe guttata (XP 012848364.1 and A0A022RE93), Eucalyptus grandis (XP 010063282.1, A0A059A781, and A0A059BWM0), Eutrema salsugineum (XP 006407255.1 and V4N4A5), Fagus sylvatica (A0A2N9IKY4 and A0A2N9IRE2), Fragaria vesca subsp. vesca (XP 011459285.1 and XP 011459286.1), Glycine max (XP 014626277.1, XP 014626279.1, A0A0R0L966, and K7KBM1), Glycine soja (XP 028221673.1, XP 028221681.1, A0A445LW28, and A0A445LW67), Gossypium arboreum (XP 017641115.1 and A0A6P4QA03), Gossypium australe (A0A5B6VE01, A0A5B6VEA5, and A0A5B6VEE8), Gossypium barbadense (A0A2P5X3D7, A0A5J5SLP4, A0A5J5SLP4, and A0A5J5WVA6), Gossypium darwinii (A0A5D2DNF7, A0A5D2GWH7, and A0A5D2HJU0), Gossypium hirsutum (XP 016706619.1 and A0A1U8IMW3), Gossypium mustelinum (A0A5D2VDK6, A0A5D2W572, A0A5D2ZQN4, and A0A5D3AFK6), Gossypium raimondii (XP 012461648.1, XP 012461655.1, XP 012461661.1, and XP 012461665.1), Gossypium tomentosum (A0A5D2QXD9), Helianthus annuus (XP 022039706.1 and A0A251UV31), Herrania umbratica (XP 021288280.1), Hevea brasiliensis (XP 021679187.1, XP 021679188.1, and XP 021679189.1), Hibiscus syriacus (A0A6A2WLV6), Ipomoea nil (XP 019196551.1), Ipomoea triloba (XP 031120100.1), Jatropha curcas (XP 012071023.1, XP 020534596.1, and XP 020534597.1), Juglans regia (XP 035543140.1 and XP 035543141.1), Lactuca saligna (A0A6S7MUU9 and A0A6S7NTJ2), Lactuca sativa (XP 023738234.1), Lupinus albus (A0A6A5LKY5), Lupinus angustifolius (XP 019435683.1, XP 019435684.1, and A0A1J7H569), Malus baccata (A0A540NQ94), Malus domestica (XP 008385780.2 and XP 028944671.1), Manihot esculenta (XP 021593564.1, XP 021593565.1, XP 021593566.1, XP 021593567.1, and XP 021593570.1), Medicago truncatula (XP 003617751.3 and XP 024640818.1), Microthlaspi erraticum (A0A6D2HZI3), Mikania micrantha (A0A5N6P041), Momordica charantia (XP 022139661.1 and A0A6J1CR74), Morus notabilis (XP 024027460.1, W9QQJ3, and W9S2T3), Nicotiana attenuata (XP 019249871.1, A0A1J6I692, and A0A1J6K801), Nicotiana tabacum (XP 016452169.1, XP 016452170.1, and A0A1S4DIR2), Nicotiana tabacum (XP 016452169.1, XP 016452170.1, and A0A1S4DIR2), Nicotiana tomentosiformis (XP 009602983.1), Olea europaea var. sylvestris (XP 022852588.1, XP 022852589.1, and XP 022852590.1), Parasponia andersonii (A0A2P5A7F0 and A0A2P5AA11), Phaseolus vulgaris (XP 007142412.1 and V7BI00), Populus alba (XP 034925199.1), Populus davidiana (A0A6M2ETT2), Populus euphratica (XP 011038778.1), Populus trichocarpa (XP 002300376.1 and XP 024457141.1), Prunus armeniaca (A0A6J5UNU4 and A0A6J5XBW8), Prunus avium (XP 021831855.1, A0A6P5TTD7, and A0A6P5TXT2), Prunus dulcis (XP 034211798.1), Prunus mume (XP 008244585.1), Prunus persica (XP 020417013.1 and A0A251PQD9), Prunus yedoensis var. nudiflora (A0A314UNP6, A0A314UXC3, and A0A314ZBK5), Punica granatum (XP 031404294.1, A0A218X8B6, and A0A2I0IQA4), Pyrus ussuriensis x Pyrus communis (A0A5N5GPD3), Quercus lobata (XP 030932844.1 and XP 030932845.1), Quercus suber (XP 023895019.1, XP 023913243.1, and XP 023925890.1), Raphanus sativus (XP 018441839.1 and A0A6J0KD93), Rhodamnia argentea (XP 030525495.1, XP 030525505.1, XP 030525514.1, XP 030525521.1, and XP 030525528.1), Ricinus communis (XP 025013782.1 and B9SGS8), Rosa chinensis (XP 024198927.1, XP 024198929.1, XP 024198930.1, and XP 024198931.1), Rubus occidentalis (MAKER: Ro01 G11692 and Ro01 G11692), Salix viminalis (A0A6N2NHD3 and A0A6N2NKI2), Salvia splendens (A0A4D8YC65, A0A4D8ZZR8, and A0A4D9AXY9), Sesamum indicum (XP 020553941.1, XP 020553942.1, and A0A619T371), Solanum chilense (A0A6N2BFW0, A0A6N2BLM4, and A0A6N2BMN4), Solanum lycopersicum (XP 010324270.1, XP 010324271.1, XP 010324270.1, and A0A3Q7HFV0), Solanum pennellii (XP 015081115.2 and XP 027774042.1), Solanum tuberosum (XP 006346146.1, M1C0B8, and M1CP72), Spinacia oleracea (XP 021842648.1, XP 021852265.1, and A0A0K9RYE3), Striga asiatica (A0A5A7RJ94), Syzygium oleosum (XP 030445495.1), Tarenaya hassleriana (XP 010534802.1 and XP 010534803.1), Theobroma cacao (XP 017980095.1, A0A061ERS9, and A0A061F4U0), Trema orientale (A0A2P5FXF3), Trifolium pratense (A0A2K3NBZ7 and A0A2K3P5K7), Vaccinium corymbosum (MAKER: VaccDscaff83-snap-gene-5.35-mRNA-1, VaccDscaff1-snap-gene-449.39-mRNA-1, and VaccDscaff83-snap-gene-5.35-mRNA-1), Vigna angularis (XP 017428460.1), Vigna angularis (XP 017428460.1), Vigna angularis var. angularis (A0A0S3SSP5), Vigna radiata var. radiata (XP 014504358.1, XP 014504359.2, XP 014504360.1, XP 022637507.1, XP 022637508.1, and XP 022637510.1), Vigna unguiculata (XP 027939534.1, A0A4D6MM30, and A0A4D6MVK0), Vitis vinifera (XP 002264649.1, XP 010644556.1, XP 010644557.1, XP 010644559.1, XP 010644561.1, and XP 019072493.1), and Ziziphus jujuba (XP 015867176.2, XP 024924549.1, and A0A6P6FSM0). Exemplary SPO11-1 gene products from monocots include those of, by way of example, Aegilops tauschii (M8BE82), Ananas comosus (XP 020105653.1, XP 020105654.1, and A0A6P5GK48), Ananas comosus var. bracteatus (A0A6V7NXT1), Asparagus officinalis (XP 020269854.1, XP 020269855.1, and XP 020269856.1), Brachypodium distachyon (XP 003559449.1, XP 014754736.1, XP 003559449.1, and A0A2K2DIL6), Dendrobium catenatum (XP 028555371.1 and XP 028555373.1), Dichanthelium oligosanthes (A0A1E5VA93), Elaeis guineensis var. tenera (A0A6I9S512), Eragrostis curvula (A0A5J9WHW8), Hordeum vulgare subsp. vulgare (A0A287SD60), Leersia perrieri (A0A0D9VZ36, A0A0D9VZ37, and A0A0D9VZ38), Musa acuminata subsp. malaccensis (XP 009380133.2 and M0RJD1), Musa balbisiana (A0A4S8ITE6 and A0A4S8IXT9), Oryza barthii (A0A0D3FH28), Oryza brachyantha (XP 006650598.1 and J3LMN4), Oryza glaberrima (I1PFJ8), Oryza glumipatula (A0A0D9Z5M7), Oryza meridionalis (A0A0E0D6X0, A0A0E0D6X1, and A0A0E0D6X2), Oryza meyeriana var. granulata (A0A6G1CUX7), Oryza nivara (A0A0E0GKX2), Oryza punctata (A0A0E0KJ43, A0A0E0KJ44, and A0A0E0KJ45), Oryza rufipogon (A0A0E0P1F0), Oryza sativa Japonica Group (XP 015630525.1 and XP 015630525.1), Oryza sativa subsp. japonica (Q7Y021), Panicum hallii (XP 025796981.1), Panicum hallii var. hallii (A0A2T7C180), Panicum miliaceum (A0A3L6SHI6, A0A3L6TKY2, and A0A3L6TVT7), Phalaenopsis equestris (XP 020583822.1), Phoenix dactylifera (A0A2H3XUN8), Setaria italica (XP 004981745.1 and K4AJ98), Setaria viridis (XP 034574051.1), Sorghum bicolor (XP 002466440.1 and XP 021304292.1), Triticum aestivum (A0A3B6KM14, A0A3B6KPH2, A0A3B6LTD1, A0A3B6LTD1, and A0A3B6MY04), Triticum urartu (M8B0H0), Zea luxurians (A0A1P8W173), Zea mays (NP 001347894.1, XP 008643457.1, XP 008643458.1, XP 008643459.1, XP 020408860.1, and XP 008643458.1), Zea mays subsp. huehuetenangensis (A0A1P8W126), Zea mays subsp. mays (A0A1P8W137 and A0A1P8W169), Zea mays subsp. mexicana (A0A1P8W110), Zea mays subsp. parviglumis (A0A1P8W0Z9, A0A1P8W103, A0A1P8W125, and A0A1P8W189), and Zostera marina (A0A0K9NY77).

A gene product of the component of DNA double strand breakage during meiotic recombination is also exemplified by a SPO11-2 protein. There is an abundance of known SPO11-2 gene products from both dicotyledonous plants (dicots) and monocotyledonous plants (monocots) including all of the following, which sequences are hereby incorporated by reference in their form as of the effective filing date. Exemplary SPO11-2 gene products from dicots include those of, by way of example, Rubus occidentalis (MAKER: Ro01_G11692, Ro05_G11480 and Ro03_G32130), Vaccinium corymbosum (MAKER: VaccDscaff28-augustus-gene-36.35-mRNA-1, VaccDscaff20-snap-gene-337.43-mRNA-1 and VaccDscaff44-snap-gene-28.39-mRNA-1), Arabidopsis lyrata (XP 020891603.1, D7KSX2 and D7M7W5), Raphanus sativus (XP 018454277.1, XP 018445957.1 and A0A6J0L2A1), Camelina sativa (XP 010473643.1, XP 010430511.1 and XP 010418437.1), Eutrema salsugineum (XP 006391688.1, XP 006398738.1 and V4KCX5), Arabidopsis thaliana (NP_176582.2, NP_001320320.1 and A0A5S9WPD6), Brassica rapa (XP 033134086.1 and NP_001288841.1), Brassica napus (XP 013747638.1, XP 013686111.1 and A0A078H9Q2), Brassica oleracea (XP 013606274.1 and A0A0D3EI93), Pistacia vera (XP 031249563.1, XP 031249562.1 and XP 031256919.1), Tarenaya hassleriana (XP 010553537.1), Quercus lobata (XP 030947178.1, XP 030947179.1 and XP 030947180.1), Theobroma cacao (XP 017973945.1, XP 017973946.1 and XP 017976926.1), Gossypium raimondii (XP 012477947.1, XP 012477948.1 and XP 012443372.1), Herrania umbratica (XP 021285184.1 and A0A6J1AEV8), Camellia sinensis (XP 028077335.1, XP 028077336.1 and XP 028091866.1), Citrus clementina (XP 024045052.1, V4TS49 and V4S460), Juglans regia (XP 018833720.1, XP 018835389.1 and A0A2I4FPY8), Manihot esculenta (XP 021625602.1, XP 021621829.1 and A0A2C9V2Z0), Citrus sinensis (XP 006476493.1, XP 006476492.1 and A0A067GLX9), Durio zibethinus (XP 022738353.1, XP 022754105.1 and XP 022742978.1), Punica granatum (XP 031390820.1, XP 031396408.1 and A0A6P8D340), Gossypium arboreum (XP 017625750.1, XP 017648982.1 and A0A6P4P284), Populus trichocarpa (XP 024449776.1, XP 024449777.1 and XP 024449778.1), Populus alba (XP 034894736.1, XP 034914178.1 and A0A4U5PMB5), Jatropha curcas (XP 012075842.1, XP 020536122.1 and XP 012082258.1), Carica papaya (XP 021887358.1, XP 021899420.1 and A0A024AG98), Vitis vinifera (XP 010658382.2, XP 002274790.1 and D7TV67), Populus euphratica (XP 011029359.1, XP 011029357.1 and XP 011029358.1), Vitis riparia (XP 034672744.1 and XP 034693410.1), Abrus precatorius (XP 027362147.1, XP 027362146.1 and XP 027366719.1), Prunus dulcis (XP 034217725.1, A0A5E4FVA8 and A0A4Y1RHN5), Prunus persica (XP 007208791.1, M5WT88 and M5WPC1), Coffea eugenioides (XP 027183324.1), Daucus carota (XP 017232138.1, A0A166F9S8 and A0A165ZWW5), Coffea arabica (XP 027080748.1, XP 027080749.1 and A0A6P6TQJ0), Prunus avium (XP 021820855.1, XP 021800639.1 and A0A6P5SVH9), Prunus mume (XP 008238957.1 and XP 008242907.2), Momordica charantia (XP 022157158.1, XP 022157159.1 and XP 022157160.1), Cucurbita maxima (XP 022995805.1, XP 022995806.1 and XP 022995807.1), Morus notabilis (XP 024031979.1, W9SZE8 and W9S2T3), Cicer arietinum (XP 004511556.1, XP 012574473.1 and XP 027193401.1), Cynara cardunculus (XP 024984774.1, XP 024986591.1 and A0A103XGB3), Medicago truncatula (XP 003611060.2, XP 024634514.1 and G7KC02), Cucurbita pepo (XP 023533741.1, XP 023533744.1 and XP 023533742.1), Fragaria vesca (XP 011463923.1), Cucurbita moschata (XP 022958585.1, XP 022958586.1 and XP 022958589.1), Helianthus annuus (XP 022022860.1, XP 022022867.1 and XP 022022872.1), Lactuca sativa (XP 023752623.1, XP 023744933.1 and A0A2J6M323), Solanum tuberosum (XP 006344018.1, XP 006367265.1 and M1CP72), Cannabis sativa (XP 030501012.1, XP 030501013.1 and XP 030501014.1), Syzygium oleosum (XP 030474966.1 and XP 030451851.1), Eucalyptus grandis (XP 010055791.1, XP 010042606.1 and XP 010037922.1), Nicotiana tomentosiformis (XP 009590057.1 and XP 009590058.1), Quercus suber (XP 023870392.1 and XP 023905057.1), Nicotiana tabacum (XP 016503496.1, XP 016503497.1 and XP 016449870.1), Vigna radiata (XP 022631755.1, XP 014520567.1 and XP 014502545.1), Malus domestica (XP 008348568.2, XP 017181650.2 and XP 017183319.1), Cajanus cajan (XP 020216133.1, XP 029127759.1 and XP 029127760.1), Solanum lycopersicum (XP 019070857.1, A0A3Q7HUG7 and A0A3Q7HXY6), Vigna unguiculata (XP 027914852.1, A0A4D6LT01 and A0A4D6MVK0), Rosa chinensis (XP 024170231.1, A0A2P6P6M3 and A0A2P6RRG4), Capsicum annuum (XP 016552705.1, XP 016542465.1 and A0A1U8FAI0), Ipomoea nil (XP 019160406.1 and XP 019151879.1), Lupinus angustifolius (XP 019422922.1, XP 019462170.1 and A0A1J7GRD7), Phaseolus vulgaris (XP 007158532.1, XP 007144519.1 and V7CNM9), Sesamum indicum (XP 011080904.1, XP 011077390.1 and A0A6I9TBC3), Nicotiana sylvestris (XP 009768829.1, XP 009768828.1 and XP 009768827.1), Solanum pennellii (XP 015084761.1), Chenopodium quinoa (XP 021740829.1, XP 021724298.1 and XP 021730665.1), Vigna angularis (XP 017407238.1 and XP 017407075.1), Ipomoea triloba (XP 031123763.1 and XP 031128351.1), Arachis duranensis (XP 015949945.1, A0A6P4CE65 and A0A6P4CEH6), Spinacia oleracea (XP 021839480.1, XP 021852265.1 and A0A0K9R4R5), Beta vulgaris (XP 010672864.1, XP 019104225.1 and XP 010696355.2), Hevea brasiliensis (XP 021672045.1, XP 021666867.1 and A0A6A6MZ35), Erythranthe guttata (XP 012854289.1, XP 012835983.1 and A0A022Q8C3), Nicotiana attenuata (XP 019224636.1, XP 019239263.1 and A0A314KV39), Gossypium hirsutum (XP 016744024.1, XP 016679456.1 and A0A1U8P1D3), Olea europaea (XP 022849710.1 and XP 022887304.1), Ziziphus jujuba (XP 024935237.1, XP 024935238.1 and XP 015899612.2), Ricinus communis (XP 002537001.2, XP 002525197.1 and B9TFI2), Glycine soja (XP 028195535.1, XP 028185830.1 and A0A445HVU6), Glycine max (XP 003542493.1, XP 003537243.1 and K7LMM4), Rhodamnia argentea (XP 030542504.1), Cucumis melo (XP 008447225.1, A0A1S3AUC6 and A0A1S3BGY2), Cucumis sativus (XP 004142105.1, A0A0A0KNU2 and A0A0A0L216), Pyrus x (XP 009333608.1), Arabis alpina (A0A087HEC6 and A0A087G4D1), Microthlaspi erraticum (A0A6D2L2S7 and A0A6D2HZI3), Arabis nemorensis (A0A565AXE8 and A0A565CHC5), Brassica campestris (A0A397XTA5, A0A3P5YDV1 and A0A024AHI2), Brassica rapa (M4EFW1 and M4CZB7), Brassica oleracea (A0A3P6DPC1 and A0A3P6EHF6), Brassica cretica (A0A3N6TIJ7 and A0A3N6Q3Z7), Gossypium barbadense (A0A5J5QDP8, A0A5J5UR31 and A0A2P5Y808), Gossypium mustelinum (A0A5D2TVK8, A0A5D2TY42 and A0A5D2Y8T5), Acer yangbiense (A0A5C7H249 and A0A5C7HSF7), Gossypium tomentosum (A0A5D2JU46, A0A5D2PFE8 and A0A5D2JV52), Trema orientale (A0A2P5FJ06 and A0A2P5FXF3), Gossypium darwinii (A0A5D2BK52, A0A5D2FJS3 and A0A5D2GWH7), Cephalotus follicularis (A0A1Q3C7R3 and A0A1Q3CVM9), Prunus yedoensis (A0A314XPY9 and A0A314ZHF4), Gossypium australe (A0A5B6VMT7), Fagus sylvatica (A0A2N9IQ11 and A0A2N9IKY4), Parasponia andersonii (A0A2P5ATM1 and A0A2P5A7F0), Prunus armeniaca (A0A6J5UXW0, A0A6J5XCS1 and A0A6J5XNT4), Nyssa sinensis (A0A5J4ZPQ9), Carpinus fangiana (A0A5N6R5K6 and A0A5N6RMK4), Citrus unshiu (A0A2H5NG77 and A0A2H5PDB2), Actinidia chinensis (A0A2R6QN91 and A0A2R6RYB5), Lactuca saligna (A0A6S7NN92 and A0A6S7NTJ2), Vigna angularis (A0A0S3T5Y2 and A0A0S3SSP5), Arachis hypogaea (A0A445ECG1, A0A445ECK6 and A0A445ECW7), Coffea canephora (A0A068UUH6 and A0A068UEN2), Cucumis melo (A0A5A7TMD5 and A0A5A7TWM5), Salix viminalis (A0A6N2KSL2, A0A6N2M3W0 and A0A6N2L9L8), Solanum chilense (A0A6N2BBQ9 and A0A6N2BFW0), Pyrus ussuriensis (A0A5N5G4N7), Cuscuta australis (A0A328E693 and A0A328DU25), Corchorus capsularis (A0A1R3IAK7 and A0A1R3HQV9), Lupinus albus (A0A6A5MWJ8, A0A6A4P4P2 and A0A6A5LKY5), Malus baccata (A0A540KB06), Salvia splendens (A0A4D8Y8A5, A0A4D8ZJA7 and A0A4D8YC65), Capsicum chinense (A0A2G3DAR1 and A0A2G3BJL6), Dorcoceras hygrometricum (A0A2Z7BHS8 and A0A2Z7CYH2), Capsicum baccatum (A0A2G2XJJ8 and A0A2G2VZF9), Hibiscus syriacus (A0A6A3B558, A0A6A3BUY1 and A0A6A2WLV6), Striga asiatica (A0A5A7QB91, A0A5A7PCB6 and A0A5A7RJ94), Corchorus olitorius (A0A1R3KG55 and A0A1R3HTH1), Cuscuta campestris (A0A484MFP1, A0A484M2M7 and A0A484N6Q1), Camellia sinensis (A0A4S4E4W4 and A0A4S4DMG8), Salix brachista (A0A5N5P2V8 and A0A5N5MDT4), Trifolium pratense (A0A2K3PS02 and A0A2K3NBZ7), Mikania micrantha (A0A5N6NXS8 and A0A5N6P041), Solanum chacoense (A0A0V0I1Z4), Populus davidiana (A0A6M2EKW0), Phaseolus angularis (A0A0L9T9E8), Citrullus lanatus (MAKER: Cla97C04G075440.1, Cla97C01G002360.1 and ClCG09G021210.1). Exemplary SPO11-2 gene products from monocots include those of, by way of example, Oryza sativa (XP 015650817.1 and XP 015628111.2), Oryza brachyantha (XP 006659133.1, XP 015695820.1 and J3MQI6), Aegilops tauschii (XP 020170313.1), Brachypodium distachyon (XP 003571458.1, XP 014756293.1 and XP 010228760.1), Setaria italica (XP 004972804.1, K3YI39 and K4AJ98), Setaria viridis (XP 034598803.1, A0A4U6U247 and A0A4U6T7D2), Panicum hallii (XP 025820114.1, XP 025796251.1 and A0A2S3I0G6), Sorghum bicolor (XP 021320245.1, XP 002468055.1 and A0A1B6PFK1), Ananas comosus (XP 020114835.1, XP 020114836.1 and XP 020108351.1), Zea mays (XP 020406911.1, NP_001298099.1 and NP_001141583.1), Phoenix dactylifera (XP 008794782.1, A0A2H3Y7W5 and A0A2H3XUN8), Asparagus officinalis (XP 020245397.1, XP 020245400.1 and A0A5P1FPI7), Musa acuminata (XP 009385844.2, XP 018676843.1 and XP 009385843.2), Dendrobium catenatum (XP 020685355.1, XP 020688215.1 and A0A2I0WIL8), Elaeis guineensis (XP 010941153.1 and XP 010941154.1), Phalaenopsis equestris (XP 020579272.1, XP 020579267.1 and XP 020591227.1), Oryza sativa (Q6ZD95, Q6ZD94 and A0A0N7KPA8), Oryza glaberrima (I1QFT0 and I1PA68), Oryza sativa (Q5ZPI9, B8BAW2 and A2XFC1), Oryza meyeriana (A0A6G1CKL2 and A0A6G1CUX7), Oryza rufipogon (A0A0E0QEJ2 and A0A0E0NTI7), Oryza nivara (A0A0E0I7D8 and A0A0E0GKX2), Oryza glumipatula (A0A0E0AR05 and A0A0D9Z5M7), Oryza barthii (A0A0D3GWH9 and A0A0D3FH28), Oryza meridionalis (A0A0E0EI30), Panicum miliaceum (A0A3L6RJM3, A0A3L6PPK4 and A0A3L6TVT7), Triticum aestivum (A0A3B6TSG3, A0A3B6SFG9 and A0A3B6TK94), Aegilops tauschii (A0A453RXC3, A0A453RXB9 and A0A453RX92), Triticum turgidum (A0A446YHI1, A0A446YHA9 and A0A446XHQ6), Leersia perrieri (A0A0D9X4K8, A0A0D9X4K9 and A0A0D9VSQ5), Eragrostis curvula (A0A5J9W4I4, A0A5J9W627 and A0A5J9WHW8), Panicum hallii (A0A2T7D3N0 and A0A2T7CE30), Hordeum vulgare (M0UTZ1, A0A287WTK0 and A0A287WTJ5), Zea mays (A0A1P8W150, A0A1P8W149 and A0A1P8W163), Zea mays (A0A1P8W133, A0A1P8W171 and A0A1P8W158), Zea diploperennis (A0A1P8W179), Zea mays (A0A1P8W114), Zea luxurians (A0A1P8W147), Zea mays (A0A1P8W1E8), Aegilops tauschii (M8BEM5 and M8B3H9), Dichanthelium oligosanthes (A0A1E5V388 and A0A1E5VA93), Musa balbisiana (A0A4S8J9M4 and A0A4S8ITE6), Oryza punctata (A0A0E0LRE6 and A0A0E0KC84), Elaeis guineensis (A0A619SD72, A0A6I9SCT7 and A0A6I9S512), Zostera marina (A0A0K9NNR2 and A0A0K9NY77), Ananas comosus (A0A6V7NQI2), Triticum urartu (M8B0H0).

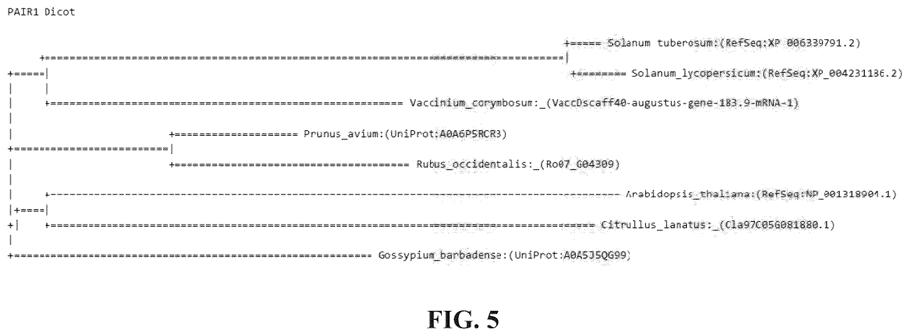

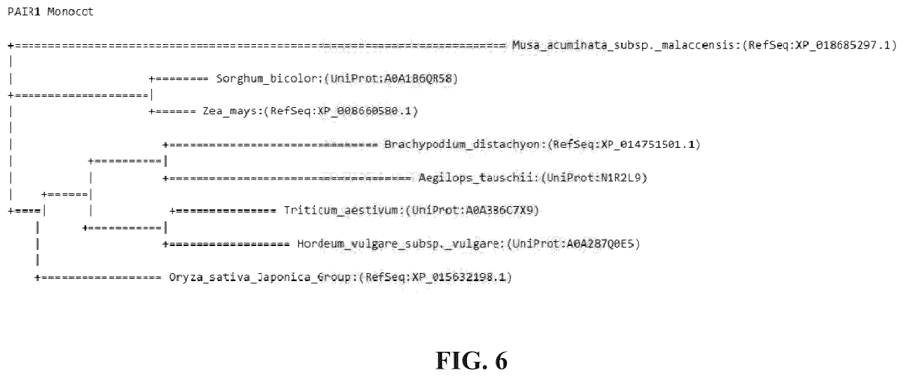

A gene product of the component of DNA double strand breakage during meiotic recombination is also exemplified by a PAIR1 protein (e.g., a PAIR1-1 protein or a PAIR1-2 protein) and specifically by the PAIR1 protein sequences, sequence alignments, and percent identities described in “MiMe Gene Product Sequences” below. Representative PAIR1 protein sequences from dicotyledonous plants (SEQ ID NOs: 37-45 and 90) are provided in the sequence listing as outlined in Table 9, including eight native sequences and a consensus sequence identified by multiple sequence alignment of the eight native sequences (Sequence Alignment 5). Table 5 shows a matrix of percent identities of the PAIR1 protein sequences from dicotyledonous plants, and a phylogenetic tree showing the relationship between the sequences is shown in FIG. 5. Representative PAIR1 protein sequences from monocotyledonous plants (SEQ ID NOs: 46-55), including a representative PAIR1-1 protein sequence from maize (SEQ ID NO: 52) and a representative PAIR1-2 protein sequence from maize (SEQ ID NO: 53), are provided in the sequence listing as outlined in Table 9, including eight native sequences and a consensus sequence identified by multiple sequence alignment of the eight native sequences (Sequence Alignment 6). Table 6 shows a matrix of percent identities of the PAIR1 protein sequences from monocotyledonous plants, and a phylogenetic tree showing the relationship between the sequences is shown in FIG. 6. The gene products of MiMe loci of the component of DNA double strand breakage during meiotic recombination includes PAIR1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the PAIR1 proteins of SEQ ID NOs: 37-55 and 90. The gene products of MiMe loci of the component of DNA double strand breakage during meiotic recombination also include: a) PAIR1-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the PAIR1-1 protein of SEQ ID NO: 52; and b) PAIR1-2 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the PAIR1-2 protein of SEQ ID NO: 53.

There is an abundance of known PAIR1 gene products from both dicotyledonous plants (dicots) and monocotyledonous plants (monocots) including all of the following, which sequences are hereby incorporated by reference in their form as of the effective filing date. Exemplary PAIR1 gene products from dicots include those of, by way of example, Abrus precatorius (XP 027337787.1), Acer yangbiense (A0A5C7I1C8), Actinidia chinensis var. chinensis (A0A2R6QP70), Arabidopsis lyrata subsp. lyrata (XP 020869978.1, XP 020869979.1, and XP 020869980.1), Arabidopsis thaliana (NP 001322648.1, NP 001322649.1, NP 001318904.1, A0A178WB09, and A0A5S9S4M2), Arabis nemorensis (A0A565ALB5), Beta vulgaris subsp. vulgaris (XP 010683598.1), Brassica campestris (A0A397XHA4, A0A3P5YM39, and A0A3P6CSD4), Brassica cretica (A0A3N6RJY2), Brassica napus (XP 013695590.1 and XP 022544029.1), Brassica oleracea (A0A3P6E280 and A0A3P6FDV6), Brassica oleracea var. oleracea (XP 013584355.1, XP 013595872.1, and XP 013595873.1), Brassica rapa (XP 009101392.1, XP 009101393.1, and XP 009101394.1), Brassica rapa subsp. pekinensis (M4E815 and M4EWQ6), Cajanus cajan (XP 029127971.1), Camelina sativa (XP 010457092.1, XP 010474782.1, XP 010474785.1, and XP 010479952.1), Camellia sinensis (XP 028094907.1), Cannabis sativa (XP 030486742.1), Capsicum annuum (XP 016582174.1, A0A1U8HFM3, A0A1U8HLP3, and A0A2G2YXN9), Capsicum baccatum (A0A2G2W6F6), Capsicum chinense (A0A2G3BSY9), Carica papaya (XP 021906204.1, XP 021906205.1, XP 021906207.1, XP 021906208.1, XP 021906209.1, and XP 021906210.1), Carpinus fangiana (A0A5N6RQI5), Cephalotus follicularis (A0A1Q3CNB3), Chenopodium quinoa (XP 021767086.1), Citrullus lanatus (MAKER: Cla97C05G081880.1, Cla97C05G081880.1, and CICG05G001510.1), Coffea arabica (A0A6P6W1B4 and A0A6P6WJ16), Coffea canephora (A0A068UKQ8), Coffea eugenioides (XP 027158697.1), Corchorus capsularis (A0A1R3I852), Corchorus olitorius (A0A1R3JZX6), Cucumis melo (A0A1S3AV35, A0A1S3AVJ5, and A0A1S4DSK1), Cucumis melo var. makuwa (A0A5D3D1V5), Cucumis sativus (XP 004134325.2, A0A0A0L487), Cucurbita maxima (XP 022974598.1, XP 022974599.1, and A0A6J1IBT5), Cucurbita moschata (A0A6J1FTV4 and A0A6J1FY69), Cucurbita pepo subsp. pepo (XP 023526419.1, XP 023526420.1, and XP 023539314.1), Cuscuta australis (A0A328DKP6), Cuscuta campestris (A0A484N2L1), Cynara cardunculus var. scolymus (XP 024988920.1, XP 024988921.1, and A0A103XUN9), Daucus carota subsp. sativus (A0A166E4T0), Dorcoceras hygrometricum (A0A2Z7BYT8), Durio zibethinus (XP 022757352.1, XP 022757354.1, XP 022757355.1, XP 022757356.1, and XP 022770442.1), Erythranthe guttata (XP 012843740.1), Eucalyptus grandis (XP 018728256.1), Eutrema salsugineum (XP 024009089.1, XP 024009090.1, and XP 024009091.1), Fagus sylvatica (A0A2N9FIT6), Fragaria vesca subsp. vesca (XP 011469013.1), Glycine max (XP 006583295.1 and XP 014633302.1), Glycine soja (XP 028223863.1, XP 028240806.1, XP 028240807.1, XP 028240808.1, and A0A0B2R526), Gossypium arboreum (XP 017633300.1), Gossypium australe (A0A5B6VK20), Gossypium barbadense (A0A5J5QG99 and A0A5J5TRX7), Gossypium darwinii (A0A5D2BMR6 and A0A5D2FL16), Gossypium hirsutum (XP 016681134.1 and XP 016722173.1), Gossypium mustelinum (A0A5D2TY94 and A0A5D2Y9I1), Gossypium raimondii (XP 012475211.1), Gossypium tomentosum (A0A5D2JY81 and A0A5D2PI96), Helianthus annuus (XP 022017690.1, XP 022017691.1, and A0A251S1G8), Herrania umbratica (XP 021280879.1 and A0A6J1A1N3), Hevea brasiliensis (XP 021673915.1 and XP 021674689.1), Hibiscus syriacus (A0A6A2Z0P2 and A0A6A3AYH3), Ipomoea nil (XP 019176166.1), Ipomoea triloba (XP 031127609.1), Jatropha curcas (XP 012083384.1), Juglans regia (XP 018831521.1, XP 035543693.1, and A0A6P9EM20), Lupinus albus (A0A6A5LPF6), Malus baccata (A0A540MP23 and A0A540N6V0), Malus domestica (XP 028951453.1, A0A498JEY0, and A0A498KIH3), Manihot esculenta (XP 021612486.1), Microthlaspi erraticum (A0A6D2IHH2), Mikania micrantha (A0A5N6NPY5), Momordica charantia (XP 022147454.1 and A0A6J1D1C7), Morella rubra (A0A6A1UPY8), Morus notabilis (XP 024027690.1 and W9S0R0), Nicotiana attenuata (XP 019264361.1, XP 019264412.1, and XP 019264465.1), Nicotiana sylvestris (A0A1U7Y6E4), Nicotiana tabacum (XP 016510267.1 and A0A1S4BT33), Nicotiana tomentosiformis (XP 009629849.1), Nyssa sinensis (A0A5J4ZSD1), Olea europaea var. sylvestris (XP 022865661.1), Parasponia andersonii (A0A2P5B2P4), Phaseolus vulgaris (V7AP39 and V7AR22), Pistacia vera (XP 031275855.1), Populus alba (XP 034904988.1 and XP 034926349.1), Populus euphratica (XP 011023330.1 and XP 011036377.1), Populus trichocarpa (A0A2K1XSF4 and A0A3N7EJ38), Prunus armeniaca (A0A6J5WM88), Prunus avium (A0A6P5R749 and A0A6P5RCR3), Prunus dulcis (XP 034205862.1 and XP 034205863.1), Prunus mume (XP 016650015.1), Prunus persica (XP 020413624.1), Prunus yedoensis var. nudiflora (A0A314ZIE6), Punica granatum (A0A218VV23), Pyrus ussuriensis x Pyrus communis (A0A5N5F3A6 and A0A5N5F3P9), Pyrus x bretschneideri (XP 009367878.1 and XP 018499250.1), Quercus lobata (XP 030932640.1), Quercus suber (XP 023873997.1), Raphanus sativus (XP 018463919.1, XP 018486658.1, and A0A6J0LZD1), Rhodamnia argentea (XP 030540885.1), Ricinus communis (XP 025015843.1), Rosa chinensis (XP 024175876.1, XP 024175877.1, and XP 024175878.1), Rubus occidentalis (MAKER: Ro07 G04309 and Ro07 G04309), Salix brachista (A0A5N5KB20), Salix viminalis (A0A6N2LNW2), Salvia splendens (A0A4D9BAV4), Sesamum indicum (XP 020552535.1 and A0A6I9U5Z0), Solanum chilense (A0A6N2BRD5), Solanum lycopersicum (XP 004231186.2 and A0A3Q7F5D7), Solanum pennellii (XP 027768349.1, XP 027768353.1, XP 027768355.1, and XP 027768356.1), Solanum tuberosum (XP 006339791.2 and M0ZGUS), Spinacia oleracea (A0A0K9R9Y9), Striga asiatica (A0A5A7QK29), Syzygium oleosum (XP 030457174.1 and XP 030457175.1), Tarenaya hassleriana (XP 019059666.1 and XP 019059667.1), Theobroma cacao (XP 007051794.2, A0A061DU76, and A0A061DV47), Trema orientale (A0A2P5F2R8), Vaccinium corymbosum (MAKER: VaccDscaff40-augustus-gene-183.9-mRNA-1), Vigna radiata var. radiata (XP 022641935.1), Vigna unguiculata (XP 027905967.1 and A0A4D6MQ75), Vitis riparia (XP 034709611.1), Vitis vinifera (XP 034709611.1 and A0A438EH38), and Ziziphus jujuba (A0A6P4AVF2 and A0A6P6FJE2). Exemplary PAIR1 gene products from monocots include those of, by way of example, Aegilops tauschii (N1R2L9), Ananas comosus (XP 020093858.1), Asparagus officinalis (XP 020249829.1 and A0A1R3L7C7), Brachypodium distachyon (XP 014751501.1), Dendrobium catenatum (XP 020700825.2 and XP 028548904.1), Dichanthelium oligosanthes (A0A1E5UL59), Elaeis guineensis (XP 010904555.1, XP 019701684.1, and XP 029116692.1), Eragrostis curvula (A0A5J9WN41), Hordeum vulgare subsp. vulgare (A0A287Q0E5), Leersia perrieri (A0A0D9VNC8), Musa acuminata subsp. malaccensis (XP 018685297.1 and M0TPK3), Musa balbisiana (A0A4S8J1F7 and A0A4S8K2U2), Oryza barthii (A0A0D3FCP7), Oryza brachyantha (XP 015690592.1, XP 015690593.1, and J3LJ21), Oryza glaberrima (I1P6N1), Oryza glumipatula (A0A0D9Z0V5), Oryza meridionalis (A0A0E0CTQ0 and A0A0E0CTQ1), Oryza meyeriana var. granulata (A0A6G1F7H0), Oryza nivara (A0A0E0GFW4), Oryza punctata (A0A0E0K7K5 and A0A0E0K7K6), Oryza sativa Japonica Group (XP 015632198.1 and XP 015632198.1), Oryza sativa subsp. indica (B8ALD4), Oryza sativa subsp. japonica (Q10T00), Panicum hallii (XP 025793246.1 and A0A2T8I6T8), Panicum hallii var. hallii (A0A2T7CIW1), Phalaenopsis equestris (XP 020588351.1 and XP 020588352.1), Phoenix dactylifera (XP 026656534.1), Setaria italica (A0A368SX39 and A0A368SX81), Sorghum bicolor (A0A1B6QR58), Triticum aestivum (A0A3B6C7X9), Zea mays (XP 008660580.1, A0A1D6JK92, A0A1D6JK93, A0A1D6JK94, and A0A1D6PM18), and Zostera marina (A0A1D6PM18).

A gene product of the component of DNA double strand breakage during meiotic recombination is also exemplified by a PRD1 protein. There is an abundance of known PRD1 gene products from both dicotyledonous plants (dicots) and monocotyledonous plants (monocots) including all of the following, which sequences are hereby incorporated by reference in their form as of the effective filing date. Exemplary PRD1 gene products from dicots include those of, by way of example, Rubus occidentalis (MAKER: Ro04_G15366, Ro04_G15356 and Ro04_G27585), Vaccinium corymbosum (MAKER: VaccDscaff36-snap-gene-224.38-mRNA-1, VaccDscaff4-augustus-gene-125.37-mRNA-1 and VaccDscaff9-processed-gene-82.27-mRNA-1), Arabidopsis thaliana (NP_001319933.1, NP_001329589.1 and 023277), Arabidopsis lyrata (XP 002870332.2 and D7MH60), Eutrema salsugineum (XP 024005243.1, XP 024005242.1 and V4MI03), Camelina sativa (XP 010450091.1, XP 010445456.2 and XP 010440518.1), Brassica oleracea (XP 013622071.1 and A0A0D3B840), Brassica napus (XP 013684107.1, XP 013735551.1 and XP 013680942.1), Raphanus sativus (XP 018472803.1 and A0A6J0MLL2), Brassica rapa (XP 009134221.3, XP 033142017.1 and XP 009134222.2), Tarenaya hassleriana (XP 010532711.1), Jatropha curcas (XP 012078523.1 and A0A067K8X0), Hevea brasiliensis (XP 021655379.1 and A0A6A6NL01), Carica papaya (XP 021888920.1), Prunus mume (XP 008222797.1), Juglans regia (XP 018828210.2 and A0A2I4F995), Rosa chinensis (XP 024192211.1, XP 024192720.1 and XP 024192213.1), Prunus persica (XP 020409962.1, A0A251QZ00 and M5XWU2), Malus domestica (XP 008337577.1 and A0A498JZS4), Pyrus x (XP 009373627.1), Citrus sinensis (XP 006492990.1, XP 024949085.1 and A0A067ECB0), Fragaria vesca (XP 011462840.1), Vitis vinifera (XP 002268156.2, F6I4C3 and A0A438FW22), Ricinus communis (XP 002516401.1, XP 025012650.1 and B9RRN2), Prunus dulcis (XP 034200900.1, A0A4Y1QNM3 and A0A5E4G9F1), Ziziphus jujuba (XP 015886784.1 and A0A6P4A0Q3), Citrus clementina (XP 024047144.1 and V4S1Y3), Theobroma cacao (XP 007034167.2, XP 017975720.1 and A0A061EKA8), Manihot esculenta (XP 021613523.1 and A0A2C9VY30), Vitis riparia (XP 034686680.1), Durio zibethinus (XP 022722511.1, XP 022722512.1 and XP 022722513.1), Quercus lobata (XP 030973650.1, XP 030973655.1 and XP 030973651.1), Camellia sinensis (XP 028092095.1), Gossypium arboreum (XP 017617983.1, A0A6P4MU81 and A0A6P4NU70), Gossypium hirsutum (XP 016722836.1, XP 016713923.1 and A0A1U8M7P9), Quercus suber (XP 023927695.1), Cicer arietinum (XP 012571814.1 and A0A1S3E8W6), Gossypium raimondii (XP 012481613.1 and A0A0D2R856), Cannabis sativa (XP 030508213.1 and XP 030508214.1), Morus notabilis (XP 024025980.1, XP 024025981.1 and XP 024025982.1), Medicago truncatula (XP 024634236.1, A0A396IWZ2 and G7JAU9), Abrus precatorius (XP 027337587.1), Momordica charantia (XP 022150395.1, XP 022150396.1 and XP 022150397.1), Populus alba (XP 034908089.1 and A0A4U5NLT1), Cucurbita moschata (XP 022929798.1 and A0A6J1ET85), Cucurbita pepo (XP 023547735.1), Arachis duranensis (XP 020983858.1, XP 020983859.1 and XP 020983862.1), Arachis hypogaea (XP 025693494.1, XP 025608197.1 and XP 029153530.1), Cajanus cajan (XP 020214485.1, XP 029129412.1 and A0A151U8M0), Populus trichocarpa (XP 024450891.1, A0A2K2BN87 and A0A3N7FKF3), Cucurbita maxima (XP 022992066.1 and A0A6J1JSI6), Lupinus angustifolius (XP 019447895.1, XP 019447896.1 and XP 019447897.1), Vigna radiata (XP 022631863.1 and A0A3Q0EIN4), Glycine soja (XP 028196886.1, A0A445HIG7 and A0A0B2QW57), Daucus carota (XP 017220890.1, XP 017220891.1 and A0A175YRN7), Cucumis sativus (XP 011656475.1, XP 011656476.1 and XP 031743763.1), Glycine max (XP 003537526.1 and I1LN40), Vigna angularis (XP 017405442.1), Cucumis melo (XP 008464335.1, A0A1S3CLC8 and A0A1S4E4N2), Olea europaea (XP 022869975.1, XP 022869976.1 and XP 022869977.1), Ipomoea nil (XP 019154232.1), Punica granatum (XP 031403087.1, XP 031403088.1 and A0A6P8DZS5), Herrania umbratica (XP 021291514.1, XP 021291515.1 and A0A6J1AYN3), Ipomoea triloba (XP 031091154.1), Helianthus annuus (XP 022009242.1 and A0A251SG36), Coffea eugenioides (XP 027167099.1), Eucalyptus grandis (XP 018715902.1 and A0A059B2M5), Coffea arabica (XP 027119178.1, XP 027082251.1 and A0A6P6WUP9), Syzygium oleosum (XP 030467401.1), Cynara cardunculus (XP 024990600.1 and A0A103Y8L6), Rhodamnia argentea (XP 030515926.1, XP 030518471.1 and XP 030518472.1), Spinacia oleracea (XP 021838255.1 and A0A0K9RXD9), Sesamum indicum (XP 011084192.2 and A0A6I9TLV0), Lactuca sativa (XP 023739621.1, A0A2J6MB63 and A0A2J6JWJ6), Solanum tuberosum (XP 015163123.1 and M1CA99), Beta vulgaris (XP 019103942.1, A0A0J8CRY5 and A0A0J8CM09), Nicotiana tomentosiformis (XP 009610577.1), Nicotiana tabacum (XP 016445067.1, XP 016504520.1 and A0A1S3XZD7), Solanum lycopersicum (XP 010318924.1 and A0A3Q7GFT7), Solanum pennellii (XP 015068318.1), Chenopodium quinoa (XP 021745688.1, XP 021749055.1 and XP 021749228.1), Nicotiana sylvestris (XP 009782988.1 and A0A1U7WWL4), Nicotiana attenuata (XP 019244488.1 and A0A1J6IIK2), Erythranthe guttata (XP 012835535.1 and A0A022RG51), Phaseolus vulgaris (XP 007163797.1, XP 007151616.1 and XP 007146568.1), Arachis ipaensis (XP 020964840.1 and XP 020976073.1), Vigna unguiculata (XP 027936266.1 and A0A4D6NHB4), Prunus avium (XP 021831282.1, XP 021831286.1 and XP 021804487.1), Pistacia vera (XP 031249582.1 and XP 031259955.1), Arabis nemorensis (A0A565BJY3), Microthlaspi erraticum (A0A6D2IN51), Arabis alpina (A0A087G6G6 and A0A087G6G7), Brassica oleracea (A0A3P6AIR2), Brassica campestris (A0A398A667, A0A397ZZT9 and A0A3P6A172), Brassica cretica (A0A3N6QDQ0), Brassica rapa (M4C955), Cephalotus follicularis (A0A1Q3ATU1), Prunus armeniaca (A0A6J5VXB6, A0A6J5TGW3 and A0A6J5W1G3), Parasponia andersonii (A0A2P5C3Y6), Trema orientale (A0A2P5FCM1), Prunus yedoensis (A0A314UCJ6, A0A314YIW2 and A0A314ZWU4), Acer yangbiense (A0A5C7IV22), Pyrus ussuriensis (A0A5N5HAT2 and A0A5N5H9J1), Malus baccata (A0A540MGX5), Corchorus capsularis (A0A1R3KXZ6), Citrus unshiu (A0A2H5PN82 and A0A2H5PMH4), Gossypium tomentosum (A0A5D2RDX9, A0A5D2LS03 and A0A5D2LRI7), Corchorus olitorius (A0A1R3GBQ8), Gossypium mustelinum (A0A5D3A4W2, A0A5D2VQU1 and A0A5D2YVK1), Gossypium darwinii (A0A5D2H9L4, A0A5D2DCG8 and A0A5D2CAI7), Gossypium barbadense (A0A5J5S7N9, A0A5J5WM21 and A0A2P5XLA0), Gossypium australe (A0A5B6WY20), Trifolium subterraneum (A0A2Z6M6J8), Fagus sylvatica (A0A2N9GRH1 and A0A2N9IZ46), Morella rubra (A0A6A1VLL7), Actinidia chinensis (A0A2R6RX33), Camellia sinensis (A0A4S4DL83), Lupinus albus (A0A6A5LAK1, A0A6A4NE66 and A0A6A4N293), Salix brachista (A0A5N5NSL3), Salix viminalis (A0A6N2N4A1), Phaseolus angularis (A0A0L9T4B4), Cucumis melo (A0A5D3BTV9), Hibiscus syriacus (A0A6A2XMI5, A0A6A3CUB7 and A0A6A3BET5), Coffea canephora (A0A068V5H5), Mikania micrantha (A0A5N6Q0E9), Lactuca saligna (A0A6S7M6Z7), Capsicum annuum (A0A1U8G465 and A0A2G3A1T1), Salvia splendens (A0A4D8ZHD2), Striga asiatica (A0A5A7P5V9), Solanum chilense (A0A6N2BF47), Carpinus fangiana (A0A5N6R2U1), Dorcoceras hygrometricum (A0A2Z7BMS1), Capsicum baccatum (A0A2G2VA17), Cuscuta australis (A0A328DPS4), Capsicum chinense (A0A2G3CYP2 and A0A2G3CYR0), Vigna angularis (A0A0S3S1F7 and A0A0S3S1C2), Arabidopsis lyrata (C7FD62, C7FD64 and C7FD67), Cuscuta campestris (A0A484N5Z4), Trifolium pratense (A0A2K3NTK8), Nyssa sinensis (A0A5J5BZD2, A0A5J5BZH2 and A0A5J5C2G0), Citrullus lanatus (MAKER: Cla97C08G147110.1, Cla97C10G195770.1 and C1CG10G012900.1). Exemplary PRD1 gene products from monocots include those of, by way of example, Oryza sativa (XP 015633378.1, XP 015633382.1 and XP 015633381.1), Aegilops tauschii (XP 020160356.1), Oryza brachyantha (XP 006652185.2 and J3LWV9), Setaria italica (XP 022684409.1, XP 012702864.1 and K3Y4Q5), Setaria viridis (XP 034604079.1 and A0A4U6U128), Sorghum bicolor (XP 021304407.1 and A0A1W0VU89), Panicum hallii (XP 025823973.1 and A0A2S3I5M6), Brachypodium distachyon (XP 014757920.1, A0A0Q3F1T2 and A0A2K2CU24), Elaeis guineensis (XP 010929840.1), Zea mays (XP 020399554.1, XP 020399553.1 and A0A1D6P5Q4), Phoenix dactylifera (XP 026656423.1 and A0A3Q0HNX0), Ananas comosus (XP 020107338.1, A0A199UEB9 and A0A6P5GMT0), Dendrobium catenatum (XP 028551946.1, XP 020672357.1 and XP 028551944.1), Musa acuminata (XP 018681600.1 and M0SUK1), Asparagus officinalis (XP 020247039.1, XP 020247040.1 and A0A5P1E870), Phalaenopsis equestris (XP 020571379.1 and XP 020584363.1), Oryza sativa (B9FEJ7, Q7XS73 and A0A0P0W8Z8), Oryza sativa (B8ASH2), Oryza nivara (A0A0E0GZC5), Oryza rufipogon (A0A0E0P7M8 and A0A0E0P7M7), Oryza sativa (Q01LK3), Oryza meridionalis (A0A0E0DCQ6, A0A0E0DCQ5 and A0A0E0DCQ7), Oryza glumipatula (A0A0D9ZJ90), Oryza barthii (A0A0D3FUF5 and A0A0D3FUF4), Oryza punctata (A0A0E0KP90 and A0A0E0KP91), Oryza meyeriana (A0A6G1C0B0, A0A6G1C0T6 and A0A6G1BZZ1), Leersia perrieri (A0A0D9W3K9, A0A0D9W3L1 and A0A0D9W3L0), Triticum aestivum (A0A2X0S6B3, A0A3B6AZ53 and A0A3B6C6C0), Triticum turgidum (A0A446L1U8, A0A446L1T3 and A0A446L1L6), Aegilops tauschii (A0A453BSK0, A0A453BSW4 and A0A453BSF5), Hordeum vulgare (A0A287IA04, A0A28719W2 and A0A28719X7), Dichanthelium oligosanthes (A0A1E5UVC9), Panicum hallii (A0A2T7CTK8, A0A2T7CTK0 and A0A2T7CTN0), Triticum urartu (M7YSL0), Eragrostis curvula (A0A5J9TQZ9 and A0A5J9SEX9), Aegilops tauschii (N1QTA7), Panicum miliaceum (A0A3L6Q0I6 and A0A3L6QAD5), Elaeis guineensis (A0A6I9RXD7), Ananas comosus (A0A6V7NNY5 and A0A6V7NNZ5), Zostera marina (A0A0K9PEW9).

A gene product of the component of DNA double strand breakage during meiotic recombination is also exemplified by a PRD2 protein. There is an abundance of known PRD2 gene products from both dicotyledonous plants (dicots) and monocotyledonous plants (monocots) including all of the following, which sequences are hereby incorporated by reference in their form as of the effective filing date. Exemplary PRD2 gene products from dicots include those of, by way of example, Rubus occidentalis (MAKER: Ro04_G21724, Ro01_G05038 and Ro02_G25952), Vaccinium corymbosum (MAKER: VaccDscaff61-augustus-gene-1.38-mRNA-1, VaccDscaff136-augustus-gene-1.29-mRNA-1 and VaccDscaff56-snap-gene-2.61-mRNA-1), Camelina sativa (XP 010483346.1, XP 010443500.1 and XP 010452894.1), Arabidopsis lyrata (XP 020870801.1 and D7MNW9), Arabidopsis thaliana (NP_001318822.1, F4KDF5 and A0A5S9YF85), Eutrema salsugineum (XP 006401157.1, XP 024012447.1 and V4KX90), Brassica rapa (XP 009120308.1, XP 009126806.1 and XP 033137704.1), Brassica napus (XP 022549025.1, XP 022551654.1 and XP 013722252.1), Brassica oleracea (XP 013611787.1, XP 013611786.1 and XP 013611785.1), Raphanus sativus (XP 018456353.1, XP 018456352.1 and XP 018469194.1), Tarenaya hassleriana (XP 010536971.1), Herrania umbratica (XP 021277481.1, XP 021277482.1 and A0A6J0ZSA5), Durio zibethinus (XP 022755334.1, XP 022755335.1 and XP 022755336.1), Populus trichocarpa (XP 002309357.1, XP 024459362.1 and B9HB40), Fragaria vesca (XP 004288723.1 and XP 011463338.1), Theobroma cacao (XP 007013849.2, XP 017983316.1 and XP 007013850.2), Populus euphratica (XP 011033509.1 and XP 011018054.1), Hevea brasiliensis (XP 021687654.1, XP 021639228.1 and A0A6A6MYU6), Populus alba (XP 034907855.1, XP 034907854.1 and A0A4U5MQL4), Manihot esculenta (XP 021616367.1, XP 021616366.1 and A0A2C9VPW9), Quercus lobata (XP 030932325.1), Vitis riparia (XP 034699133.1, XP 034699135.1 and XP 034699134.1), Vitis vinifera (XP 010656619.1, XP 010656620.1 and D7U1D1), Quercus suber (XP 023913538.1), Pistacia vera (XP 031247627.1 and XP 031250356.1), Jatropha curcas (XP 012071631.1 and A0A067L3R5), Prunus dulcis (XP 034222976.1, XP 034222977.1 and A0A5E4F924), Cajanus cajan (XP 020231276.1 and A0A151S9I6), Pyrus x (XP 009358423.1 and XP 009358422.1), Prunus persica (XP 007202206.1 and M5W9D0), Citrus clementina (XP 024034382.1, XP 024034381.1 and XP 024034380.1), Prunus mume (XP 008243016.1), Phaseolus vulgaris (XP 007154872.1 and V7CD94), Vigna angularis (XP 017411702.1), Malus domestica (XP 017177993.2, XP 008384229.2 and A0A498HS17), Morus notabilis (XP 010091171.1 and W9QXN7), Vigna radiata (XP 022639565.1, XP 022639566.1 and A0A3Q0F6B7), Prunus avium (XP 021814009.1, XP 021814010.1 and A0A6P5SDL4), Vigna unguiculata (XP 027919041.1 and A0A4D6KV33), Gossypium hirsutum (XP 016725521.1, A0A1U8MKI1 and A0A1U8MF99), Glycine max (XP 003550683.1, XP 003518223.3 and I1MT12), Citrus sinensis (XP 024952992.1, XP 006474022.1 and A0A067FGF8), Cicer arietinum (XP 027192370.1, XP 004508234.1 and A0A1S2YQ98), Medicago truncatula (XP 003610291.1 and G7JFK5), Glycine soja (XP 028210285.1, XP 028184860.1 and A0A445LRI7), Eucalyptus grandis (XP 010051044.1 and A0A059CSG8), Rhodamnia argentea (XP 030523639.1 and XP 030523641.1), Cucurbita maxima (XP 022969582.1 and A0A6J1HWP8), Momordica charantia (XP 022158177.1 and A0A6J1DWJ3), Cannabis sativa (XP 030509912.1 and XP 030509913.1), Solanum pennellii (XP 015080882.1, XP 027773946.1 and XP 015080883.1), Nicotiana tabacum (XP 016446597.1, XP 016441054.1 and A0A1S3Y3Z0), Arachis ipaensis (XP 016200340.1 and XP 020962020.1), Arachis hypogaea (XP 025630617.1, XP 025607528.1 and XP 025607530.1), Juglans regia (XP 035541553.1, XP 035541554.1 and XP 035541552.1), Punica granatum (XP 031387850.1, A0A6P8CV76 and A0A218VZ09), Nicotiana tomentosiformis (XP 009597540.1, XP 033511321.1 and XP 009597542.1), Nicotiana attenuata (XP 019227343.1, XP 019227344.1 and A0A314KQJ6), Abrus precatorius (XP 027368776.1), Lupinus angustifolius (XP 019441734.1 and A0A4P1RWQ4), Capsicum annuum (XP 016555696.1, XP 016555697.1 and XP 016555699.1), Lactuca sativa (XP 023730446.1, XP 023730445.1 and A0A2J6KN24), Cucumis melo (XP 008452717.1, A0A1S3BUG1 and A0A1S3BUI0), Cynara cardunculus (XP 024980680.1, XP 024980681.1 and A0A103XN14), Cucumis sativus (XP 011648897.1 and A0A0A0LK37), Helianthus annuus (XP 022016224.1, XP 022016222.1 and XP 022016220.1), Solanum tuberosum (XP 015162530.1 and M1AS84), Syzygium oleosum (XP 030458919.1), Erythranthe guttata (XP 012839854.1 and A0A022R6P0), Ricinus communis (XP 025015701.1 and B9T743), Sesamum indicum (XP 020550918.1), Coffea eugenioides (XP 027176524.1, XP 027176525.1 and XP 027176527.1), Ipomoea triloba (XP 031108698.1), Ipomoea nil (XP 019177904.1), Gossypium raimondii (XP 012464544.1, XP 012464543.1 and XP 012464545.1), Daucus carota (XP 017250480.1, XP 017250481.1 and A0A164ZNC0), Coffea arabica (XP 027066531.1, XP 027069978.1 and XP 027069981.1), Chenopodium quinoa (XP 021750265.1 and XP 021756774.1), Camellia sinensis (XP 028074469.1), Beta vulgaris (XP 010687618.1 and XP 019106900.1), Spinacia oleracea (XP 021835338.1 and A0A0K9R6R4), Nicotiana sylvestris (XP 009757955.1 and A0A1U7V845), Brassica campestris (A0A397XJY5, A0A3P6AR21 and A0A398AAW1), Microthlaspi erraticum (A0A6D2K197), Brassica rapa (M4DV63 and M4CEQ6), Brassica oleracea (A0A3P6CZT3 and A0A3P6EB31), Arabis nemorensis (A0A565CS61), Rosa chinensis (A0A2P6RI39), Fagus sylvatica (A0A2N9FXA4), Carpinus fangiana (A0A5N6RUM1 and A0ASN6L300), Gossypium tomentosum (A0A5D2MPC8, A0A5D2I1S1 and A0A5D2MP80), Gossypium arboreum (A0A6P4NB99), Cephalotus follicularis (A0A1Q3CSU0), Gossypium mustelinum (A0A5D2WLG2 and A0A5D2S6E5), Gossypium barbadense (A0A2P5VQE1 and A0A5J5NV37), Gossypium darwinii (A0A5D2E3F3 and A0A5D2A2P5), Ziziphus jujuba (A0A6P4ACY8 and A0A6P6FQ23), Morella rubra (A0A6A1VLR5 and A0A6A1X0J5), Pyrus ussuriensis (A0A5N5FNM8 and A0A5N5GYB0), Prunus yedoensis (A0A314XVL5), Actinidia chinensis (A0A2R6R4V9), Gossypium australe (A0A5B6WVQ0), Citrus unshiu (A0A2H5P867), Cucurbita moschata (A0A6J1FBV6), Solanum chilense (A0A6N2B8C6), Prunus armeniaca (A0A6J5VIX1), Arachis duranensis (A0A6P4BXY2, A0A6P4BNE6 and A0A6P4BXY8), Acer yangbiense (A0A5C7HW14), Trema orientale (A0A2P5FNN7), Parasponia andersonii (A0A2P5A5E3), Lupinus albus (A0A6A4PJK3), Cucumis melo (A0A5A7ULP1 and A0A5D3BBS1), Mikania micrantha (A0A5N6NP51), Cuscuta australis (A0A328DZA0), Hibiscus syriacus (A0A6A2Y4B9), Salvia splendens (A0A4D8ZK22), Coffea canephora (A0A068U5D3), Trifolium pratense (A0A2K3PNZ0 and A0A2K3LE33), Striga asiatica (A0A5A7Q315), Lactuca saligna (A0A6S7NBE7 and A0A6S7N8G0), Vigna angularis (A0A0S3R6E1), Solanum lycopersicum (A0A3Q7H6H1), Camellia sinensis (A0A4S4EA41), Trifolium subterraneum (A0A2Z6LPS6), Salix brachista (A0A5N5MC64), Nyssa sinensis (A0A5J5B2L5), Corchorus capsularis (A0A1R3JUF4), Brassica cretica (A0A3N6RIB4), Salix viminalis (A0A6N2N463 and A0A6N2MQY1), Cuscuta campestris (A0A484KZ46), Citrullus lanatus (MAKER: Cla97C02G040150.1, C1CG02G014390.1 and ClCG08G002210.1). Exemplary PRD2 gene products from monocots include those of, by way of example, Oryza sativa (XP 015648527.1), Zea mays (NP_001130070.1, XP 035822500.1 and A0A3L6F902), Setaria viridis (XP 034583479.1, XP 034583480.1 and XP 034600292.1), Brachypodium distachyon (XP 014755693.1, XP 014755694.1 and A0A2K2D2R0), Sorghum bicolor (XP 021320263.1 and A0A1B6PIJ6), Setaria italica (XP 022679807.1, XP 004958403.1 and A0A368Q7W7), Panicum hallii (XP 025822137.1, A0A2S3I5D3 and A0A2S3I4K3), Phoenix dactylifera (XP 026666384.1, XP 026666394.1 and A0A3Q0IHH9), Elaeis guineensis (XP 029116703.1, XP 010904625.1 and XP 029116701.1), Musa acuminata (XP 018680557.1 and M0SNE0), Asparagus officinalis (XP 020265780.1, XP 020265781.1 and A0A5P1EWQ7), Ananas comosus (XP 020100855.1, XP 020100858.1 and A0A199V516), Dendrobium catenatum (XP 028556125.1, XP 028556123.1 and XP 028556122.1), Phalaenopsis equestris (XP 020572619.1, XP 020572620.1 and XP 020572621.1), Oryza sativa (Q0J3T0, B9FYH9 and A0A0P0XI84), Oryza glaberrima (I1QKB4), Oryza sativa (B8B9F2), Oryza glumipatula (A0A0E0AYT7), Oryza meyeriana (A0A6G1CDX2 and A0A6G1CFI5), Oryza rufipogon (A0A0E0QM92), Oryza punctata (A0A0E0LXV3), Oryza barthii (A0A0D3H336), Oryza meridionalis (A0A0E0ENX5), Oryza nivara (A0A0E0IFP4), Leersia perrieri (A0A0D9XAR5), Aegilops tauschii (A0A453RRS6, A0A453RRT0 and A0A453RR12), Oryza brachyantha (J3MV74), Triticum aestivum (A0A3B6TQZ6, A0A3B6SK66 and A0A3B6RFI0), Triticum turgidum (A0A446XF03, A0A446XEY5 and A0A446YEH6), Panicum miliaceum (A0A3L6PPS2, A0A3L6RHG4 and A0A3L6SDJ5), Hordeum vulgare (F2DSH1, A0A287WMA5 and A0A287WMA8), Panicum hallii (A0A2T7DAN7, A0A2T7DAP1 and A0A2T7DAM6), Eragrostis curvula (A0A5J9SZX7), Elaeis guineensis (A0A6I9Q8D6), Aegilops tauschii (M8BZL5), Zostera marina (A0A0K9P0H1), Musa balbisiana (A0A4S8KBI9), Triticum urartu (M8A8S1), Ananas comosus (A0A6V7NWZ9).

A gene product of the component of DNA double strand breakage during meiotic recombination is also exemplified by a DFO protein. There is an abundance of known DFO gene products from both dicotyledonous plants (dicots) and monocotyledonous plants (monocots) including all of the following, which sequences are hereby incorporated by reference in their form as of the effective filing date. Exemplary DFO gene products from dicots include those of, by way of example, Rubus occidentalis (MAKER: Ro03_G26509, Ro07_G33794 and Ro07_G18082), Vaccinium corymbosum (MAKER: VaccDscaff11-snap-gene-319.30-mRNA-1, VaccDscaff24-snap-gene-66.56-mRNA-1 and VaccDscaff15-snap-gene-307.28-mRNA-1), Eutrema salsugineum (XP 006417849.2, XP 024008614.1 and V4L1Q4), Arabidopsis thaliana (NP_172187.1, NP_001322523.1 and NP_001322522.1), Arabidopsis lyrata (XP 002892378.1, XP 020868783.1 and D7KGY5), Camelina sativa (XP 010475458.1, XP 010487080.1 and XP 010487088.1), Brassica oleracea (XP 013638275.1, XP 013638274.1 and A0A0D3C874), Brassica napus (XP 022558491.1, XP 013697356.1 and A0A078F6V6), Tarenaya hassleriana (XP 010521977.1), Brassica rapa (XP 009118999.1), Raphanus sativus (XP 018437182.1 and A0A6J0JND9), Citrus sinensis (XP 006488695.1, XP 015388827.1 and A0A067FLJ6), Citrus clementina (XP 006425145.2 and V4SHS2), Ziziphus jujuba (XP 024926405.1, A0A6P6FY95 and A0A6P6FX69), Carica papaya (XP 021909747.1, XP 021909746.1 and XP 021909745.1), Durio zibethinus (XP 022729520.1, XP 022729519.1 and XP 022729518.1), Camellia sinensis (XP 028112159.1), Prunus persica (XP 020422658.1, XP 020422657.1 and XP 020422655.1), Herrania umbratica (XP 021278392.1 and A0A6J0ZUD4), Prunus avium (XP 021810585.1, XP 021810584.1 and XP 021810586.1), Daucus carota (XP 017241150.1 and A0A166CAL0), Punica granatum (XP 031385178.1, A0A218W1E8 and A0A6P8D0V1), Prunus mume (XP 016649629.1), Juglans regia (XP 018819210.1 and A0A2I4EII5), Vitis vinifera (XP 019075937.1 and F6GUL6), Theobroma cacao (XP 017983068.1 and A0A061GYP1), Malus domestica (XP 008384741.3 and XP 017191366.2), Prunus dulcis (XP 034221553.1, XP 034221552.1 and XP 034221554.1), Rosa chinensis (XP 024198844.1 and A0A2P6QKD3), Morus notabilis (XP 024023352.1, XP 024023350.1 and XP 024023351.1), Fragaria vesca (XP 011461453.1), Lactuca sativa (XP 023728783.1 and A0A2J6KRN5), Gossypium arboreum (XP 017640176.1 and A0A6P4PAT7), Populus alba (XP 034923432.1 and A0A4U5QFY2), Cynara cardunculus (XP 024981014.1 and A0A103XNW9), Populus trichocarpa (XP 024449612.1, XP 024449611.1 and A0A3N7ECL3), Cucurbita maxima (XP 022970619.1, XP 022970621.1 and A0A6J1I645), Cucurbita pepo (XP 023520164.1), Helianthus annuus (XP 021977889.1, XP 021977888.1 and A0A251U794), Sesamum indicum (XP 020550599.1), Rhodamnia argentea (XP 030537522.1, XP 030537520.1 and XP 030537519.1), Vigna unguiculata (XP 027931800.1 and A0A4D6KIV2), Populus euphratica (XP 011024898.1), Cucumis sativus (XP 004139510.2, XP 011654706.1 and A0A0A0LSV1), Jatropha curcas (XP 012080432.1 and A0A067LMV9), Cannabis sativa (XP 030505537.1), Hevea brasiliensis (XP 021637881.1 and A0A6A6M4U2), Nicotiana tabacum (XP 016495491.1, XP 016495490.1 and A0A1S4C348), Cucurbita moschata (XP 022964954.1, XP 022964955.1 and A0A6J1HPP0), Vigna angularis (XP 017411777.1), Vigna radiata (XP 014489652.1, XP 014489651.1 and XP 022632864.1), Nicotiana tomentosiformis (XP 033508346.1), Lupinus angustifolius (XP 019439257.1 and XP 019439256.1), Nicotiana attenuata (XP 019225529.1, XP 019225528.1 and A0A314KV22), Phaseolus vulgaris (XP 007146825.1 and V7BLG2), Beta vulgaris (XP 019106582.1, XP 010685902.1 and XP 010685901.1), Erythranthe guttata (XP 012839248.1 and A0A022RYV4), Eucalyptus grandis (XP 010045369.1 and A0A059D9Z2), Arachis hypogaea (XP 025626484.1, XP 025626483.1 and XP 029144829.1), Coffea eugenioides (XP 027174701.1), Pyrus x (XP 018505828.1 and XP 018505829.1), Momordica charantia (XP 022154860.1, XP 022154861.1 and A0A6J1DKU7), Cajanus cajan (XP 020224724.1, XP 029128827.1 and A0A151SY65), Pistacia vera (XP 031271877.1), Syzygium oleosum (XP 030464387.1), Abrus precatorius (XP 027336920.1), Ipomoea triloba (XP 031113591.1), Spinacia oleracea (XP 021835754.1), Solanum tuberosum (XP 006350870.1 and M1CZR2), Ipomoea nil (XP 019163004.1), Glycine soja (XP 028213990.1 and A0A445FPP7), Chenopodium quinoa (XP 021716273.1), Glycine max (XP 014625937.1 and A0A0R0F783), Arachis duranensis (XP 020985247.1 and A0A6P5MJ57), Medicago truncatula (XP 024634952.1, XP 024636021.1 and XP 024636022.1), Arachis ipaensis (XP 020962923.1), Cicer arietinum (XP 004500310.1 and A0A1S2Y8F7), Nicotiana sylvestris (XP 009760472.1, XP 009760474.1 and A0A1U7V260), Solanum lycopersicum (XP 004242497.1, XP 010323120.1 and A0A3Q7H1Y6), Capsicum annuum (XP 016576851.1 and A0A1U8H038), Solanum pennellii (XP 015078873.1 and XP 015078872.1), Olea europaea (XP 022864216.1), Cucumis melo (XP 008464293.1, A0A1S3CL48 and A0A1S3CL50), Arabis nemorensis (A0A565AML0), Arabis alpina (A0A087HLK5), Microthlaspi erraticum (A0A6D2IKS7), Brassica campestris (A0A397XPS2 and A0A3P6CU35), Brassica rapa (M4DGC6), Brassica cretica (A0A3N6S2L8), Brassica oleracea (A0A3P6FFD0), Acer yangbiense (A0A5C7IZ18), Carpinus fangiana (A0A660KVC0), Cephalotus follicularis (A0A1Q3C2Q4), Corchorus olitorius (A0A1R3K5L7), Nyssa sinensis (A0A5J5C673), Gossypium barbadense (A0A5J5S660, A0A2P5WHE3 and A0A5J5W8F4), Prunus yedoensis (A0A314UUL2 and A0A314YJK8), Actinidia chinensis (A0A2R6P6E0), Gossypium darwinii (A0A5D2H0J1 and A0A5D2D5R1), Gossypium tomentosum (A0A5D2R5S0 and A0A5D2LPC1), Corchorus capsularis (A0A1R3IJY9), Gossypium mustelinum (A0A5D2ZUG2 and A0A5D2VQ08), Pyrus ussuriensis (A0A5N5G950), Ricinus communis (B9S276), Gossypium hirsutum (A0A1U8LFZ5), Gossypium australe (A0A5B6USI9), Lactuca saligna (A0A6S7PE17), Parasponia andersonii (A0A2P5AU00), Malus baccata (A0A540KQN2), Gossypium raimondii (A0A0D2RMW7), Trema orientale (A0A2P5EJY8), Prunus armeniaca (A0A6J5XIX9 and A0A6J5V8B8), Coffea arabica (A0A6P6SD11), Vigna angularis (A0A0S3S661), Coffea canephora (A0A068ULD3), Hibiscus syriacus (A0A6A3BAL3 and A0A6A2ZCX6), Trifolium medium (A0A392MQG1), Camellia sinensis (A0A4S4DHS1 and A0A4S4ECA0), Lupinus albus (A0A6A5PBK7, A0A6A4PBZ3 and A0A6A5NCC3), Trifolium pratense (A0A2K3LL18), Striga asiatica (A0A5A7PCQ0 and A0A5A7P6J2), Cuscuta campestris (A0A484NGK7), Cuscuta australis (A0A328DDR1), Trifolium subterraneum (A0A2Z6M6H1), Salvia splendens (A0A4D8YCE7 and A0A4D8ZV37), Dorcoceras hygrometricum (A0A2Z7B2K8), Cucumis melo (A0A5A7TKX6), Salix viminalis (A0A6N2LVK2), Manihot esculenta (A0A2C9U4G3), Solanum chilense (A0A6N2BYG3), Salix brachista (A0A5N5JPH8), Citrullus lanatus (MAKER: Cla97C03G051080.1, C1CG03G000270.1 and ClCG07G011220.1). Exemplary DFO gene products from monocots include those of, by way of example, Oryza sativa (XP 015617626.1), Sorghum bicolor (XP 021301316.1, XP 002448863.2 and A0A1B6PAW4), Oryza brachyantha (XP 015697537.1, XP 015698189.1 and J3N5D3), Brachypodium distachyon (XP 010238821.1 and I1IV64), Aegilops tauschii (XP 020183945.1), Panicum hallii (XP 025805823.1, A0A2S3H5I4 and A0A2T8I6Y6), Setaria italica (XP 004978584.2, XP 022684772.1 and K3ZJY2), Zea mays (XP 020397706.1, A0A1D6QG87 and A0A3L6EXP8), Phoenix dactylifera (XP 008792172.1 and A0A2H3Y1W3), Elaeis guineensis (XP 029117217.1, XP 029117218.1 and XP 029117219.1), Ananas comosus (XP 020100636.1, XP 020100637.1 and A0A6P5G5V3), Asparagus officinalis (XP 020244612.1 and XP 020244611.1), Musa acuminata (XP 018676730.1), Dendrobium catenatum (XP 020684766.1, XP 028556367.1 and XP 028556366.1), Phalaenopsis equestris (XP 020581864.1 and XP 020581863.1), Oryza sativa (Q0IV79, B9GBF5 and B9GBE9), Oryza sativa (B8BIN1 and B8BNT9), Oryza rufipogon (A0A0E0R350, A0A0E0RCN4 and A0A0E0R351), Oryza barthii (A0A0D3HH83, A0A0D3HH82 and A0A0D3HQF7), Oryza nivara (A0A0E0J5U8), Oryza glaberrima (I1R3A8), Oryza meridionalis (A0A0E0F1K8), Oryza punctata (A0A0E0MIP5, A0A0E0MBJ2 and A0A0E0MBJ3), Oryza meyeriana (A0A6G1BPC9, A0A6G1BP32 and A0A6G1BPE6), Eragrostis curvula (A0A5J9UWH0, A0A5J9UUS8 and A0A5J9UU88), Triticum aestivum (A0A3B6KH19, A0A3B6LJZ3 and A0A3B6MPM1), Triticum turgidum (A0A446SYF6 and A0A446U3Z9), Aegilops tauschii (A0A453KE96, A0A453KEA3 and A0A453KE93), Setaria viridis (A0A4U6TAU4), Hordeum vulgare (A0A287R398, A0A287R393 and A0A287R3J9), Panicum hallii (A0A2T7E4B4 and A0A2T7CJ61), Aegilops tauschii (M8B9G0), Panicum miliaceum (A0A3L6PK61), Triticum urartu (T1M0Z3), Elaeis guineensis (A0A6I9QCM6), Ananas comosus (A0A6V7NTU1), Musa balbisiana (A0A4S8JS48), Zostera marina (A0A0K9PED1), Leersia perrieri (A0A0D9XN82 and A0A0D9XN83).

A gene product of the component of DNA double strand breakage during meiotic recombination is also exemplified by a MTOPVIB protein. There is an abundance of known MTOPVIB gene products from both dicotyledonous plants (dicots) and monocotyledonous plants (monocots) including all of the following, which sequences are hereby incorporated by reference in their form as of the effective filing date. Exemplary MTOPVIB gene products from dicots include those of, by way of example, Rubus occidentalis (MAKER: Ro04_G00308), Vaccinium corymbosum (MAKER: VaccDscaff14-snap-gene-223.38-mRNA-1, VaccDscaff2-snap-gene-259.39-mRNA-1 and VaccDscaff25-snap-gene-270.35-mRNA-1), Arabidopsis thaliana (NP_001031211.1, NP_176249.2 and NP_001031210.1), Camelina sativa (XP 010510946.1, XP 019083617.1 and XP 019101447.1), Arabidopsis lyrata (XP 020891213.1 and D7KWU3), Eutrema salsugineum (XP 024004274.1 and V4KPY5), Brassica oleracea (XP 013618081.1 and A0A0D3AAB8), Raphanus sativus (XP 018439918.1, XP 018439921.1 and XP 018439920.1), Brassica napus (XP 013672333.1, A0A078HBC9 and A0A078GHA8), Brassica rapa (XP 009103783.1, XP 009103780.1 and XP 033133728.1), Tarenaya hassleriana (XP 010559040.1, XP 010559041.1 and XP 010559042.1), Citrus sinensis (XP 006484319.1, A0A067H4A8 and A0A067GUM7), Citrus clementina (XP 024042200.1 and V4SUQ7), Theobroma cacao (XP 007044672.2 and A0A061EDY0), Herrania umbratica (XP 021293001.1 and A0A6J1B1E1), Durio zibethinus (XP 022775585.1 and A0A6P6BEV6), Malus domestica (XP 028952520.1 and A0A498HLD2), Populus euphratica (XP 011011125.1 and XP 011011126.1), Morus notabilis (XP 024023902.1 and W9RN95), Prunus avium (XP 021809717.1, XP 021809716.1 and XP 021809718.1), Rosa chinensis (XP 024194641.1, XP 024194640.1 and XP 024194642.1), Populus trichocarpa (XP 024466039.1, XP 024466040.1 and A0A3N7FKS5), Pistacia vera (XP 031277040.1, XP 031277042.1 and XP 031277041.1), Prunus persica (XP 020411080.1, XP 020411079.1 and A0A251R352), Vitis vinifera (XP 010651436.1, XP 010651449.1 and XP 010651443.1), Gossypium raimondii (XP 012450623.1, XP 012450620.1 and XP 012450621.1), Prunus dulcis (XP 034200044.1, XP 034200043.1 and XP 034200045.1), Populus alba (XP 034909967.1, XP 034909968.1 and A0A4U5MAX7), Gossypium arboreum (XP 017637986.1, XP 017637985.1 and XP 017637987.1), Hevea brasiliensis (XP 021635214.1 and A0A6A6KVU6), Ziziphus jujuba (XP 024934411.1 and A0A6P6GJY9), Prunus mume (XP 016647038.1), Cannabis sativa (XP 030496715.1), Quercus suber (XP 023884227.1, XP 023884228.1 and XP 023884229.1), Quercus lobata (XP 030960182.1, XP 030960184.1 and XP 030960185.1), Gossypium hirsutum (XP 016754896.1, XP 016754894.1 and XP 016754897.1), Ricinus communis (XP 015578627.1 and B9SH96), Vitis riparia (XP 034690650.1 and XP 034690657.1), Fragaria vesca (XP 011463199.1, XP 011463197.1 and XP 011463198.1), Juglans regia (XP 018851384.1, XP 018851385.1 and A0A2I4H5F6), Lupinus angustifolius (XP 019445722.1, XP 019445449.1 and A0A4P1RGF2), Jatropha curcas (XP 020539237.1 and A0A067K478), Daucus carota (XP 017235110.1 and XP 017235109.1), Glycine max (XP 014626005.1, XP 014626021.1 and A0A0R0L5T4), Vigna radiata (XP 022633734.1 and A0A3Q0ETG4), Momordica charantia (XP 022131879.1, XP 022131880.1 and XP 022131881.1), Vigna angularis (XP 017426321.1), Vigna unguiculata (XP 027915211.1 and A0A4D6LP09), Cicer arietinum (XP 012568210.1 and A0A1S3DXM3), Cynara cardunculus (XP 024987354.1 and A0A118JYB2), Ipomoea triloba (XP 031125077.1), Ipomoea nil (XP 019171247.1), Olea europaea (XP 022891502.1), Coffea eugenioides (XP 027152580.1 and XP 027182129.1), Punica granatum (XP 031401562.1, A0A6P8DVQ9 and A0A2I0K3Q4), Lactuca sativa (XP 023762248.1 and A0A2J6LHT0), Medicago truncatula (XP 024639573.1, G7K1C8 and A0A396HR42), Syzygium oleosum (XP 030467358.1, XP 030467359.1 and XP 030467360.1), Eucalyptus grandis (XP 010023546.1 and A0A059B2G0), Cucurbita pepo (XP 023545219.1), Arachis hypogaea (XP 025654283.1 and XP 025654285.1), Coffea arabica (XP 027098502.1, XP 027095817.1 and A0A6P6V957), Arachis duranensis (XP 015965033.1, A0A6P4DI78 and A0A6P5NQI9), Cucumis melo (XP 016900028.1, XP 016900027.1 and A0A1S4DVN5), Carica papaya (XP 021895828.1, XP 021895827.1 and XP 021895823.1), Spinacia oleracea (XP 021842936.1, XP 021842937.1 and A0A0K9RXF9), Cucurbita maxima (XP 022996892.1, XP 022996887.1 and XP 022996889.1), Rhodamnia argentea (XP 030545064.1, XP 030545061.1 and XP 030545062.1), Cucurbita moschata (XP 022962326.1, XP 022962323.1 and XP 022962325.1), Cucumis sativus (XP 011649478.1, XP 004142982.2 and XP 011649475.1), Beta vulgaris (XP 010682537.1, XP 010682538.1 and XP 010682539.1), Chenopodium quinoa (XP 021727466.1 and XP 021741594.1), Nicotiana attenuata (XP 019261468.1 and A0A314LA60), Nicotiana tomentosiformis (XP 009631009.1, XP 033508619.1 and XP 009631010.1), Solanum tuberosum (XP 015166906.1, XP 015166907.1 and M1CGP5), Nicotiana tabacum (XP 016464688.1, XP 016464690.1 and XP 016464689.1), Solanum lycopersicum (XP 010322745.1, XP 010322746.1 and XP 019069719.1), Solanum pennellii (XP 015077943.1, XP 015077944.1 and XP 027773340.1), Helianthus annuus (XP 021999156.1 and A0A251T728), Capsicum annuum (XP 016574902.1, XP 016574901.1 and XP 016574906.1), Sesamum indicum (XP 020554798.1), Nicotiana sylvestris (XP 009804019.1, A0A1U7YZ82 and A0A1U7YLN0), Camellia sinensis (XP 028100583.1 and XP 028100584.1), Erythranthe guttata (XP 012852574.1, XP 012846004.1 and A0A022QSF8), Abrus precatorius (XP 027362820.1), Manihot esculenta (XP 021633223.1, XP 021633225.1 and A0A2C9UNK2), Phaseolus vulgaris (XP 007157444.1 and V7CJ97), Glycine soja (XP 028195157.1, A0A0B2PRT8 and A0A445LY47), Arabidopsis halleri (I0J3F4), Arabis alpina (A0A087HF84), Arabis nemorensis (A0A565AX97), Microthlaspi erraticum (A0A6D2HQI2 and A0A6D2JLJ0), Brassica oleracea (A0A3P6F1G5), Brassica campestris (A0A398AWN6 and A0A3P5ZXB4), Brassica rapa (M4ERT0), Cephalotus follicularis (A0A1Q3AZR5), Malus baccata (A0A540KVD0), Citrus unshiu (A0A2H5N7Z1, A0A2H5N800 and A0A2H5N805), Gossypium darwinii (A0A5D2C8Y8, A0A5D2G567 and A0A5D2C4A2), Gossypium tomentosum (A0A5D2KH53, A0A5D2Q2M6 and A0A5D2KJL9), Gossypium barbadense (A0A5J5R1L1, A0A2P5XV59 and A0A5J5N6Z9), Gossypium mustelinum (A0A5D2UJQ4, A0A5D2YTV1 and A0A5D2UH01), Prunus yedoensis (A0A314V0L2), Nyssa sinensis (A0A5J5AJD0), Pyrus ussuriensis (A0A5N5HGZ2), Acer yangbiense (A0A5C7HGU7), Parasponia andersonii (A0A2P5BBG1), Corchorus capsularis (A0A1R3I6R3), Trema orientale (A0A2P5FME2), Gossypium australe (A0A5B6W739), Trifolium pratense (A0A2K3NRL5), Prunus armeniaca (A0A6J5TK50), Hibiscus syriacus (A0A6A2WVL9), Corchorus olitorius (A0A1R3IC84), Trifolium subterraneum (A0A2Z6NBY5), Salix viminalis (A0A6N2N392), Lupinus albus (A0A6A5MLQ6, A0A6A4QGW4 and A0A6A4QIZ2), Carpinus fangiana (A0A5N6R4E8), Striga asiatica (A0A5A7Q6F2), Salix brachista (A0A5N5L480), Brassica cretica (A0A3N6S767), Actinidia chinensis (A0A2R6P416), Solanum chilense (A0A6N2CIU3), Coffea canephora (A0A068UZZ9), Cuscuta campestris (A0A484MG17 and A0A484MXA6), Salvia splendens (A0A4D9A7J6 and A0A4D8YFN9), Cuscuta australis (A0A328E9C7), Cajanus cajan (A0A151TGJ7), Dorcoceras hygrometricum (A0A2Z7A9G4), Cucumis melo (A0A5A7V3J3), Mikania micrantha (A0A5N6NRU9), Fagus sylvatica (A0A2N9IPT0), Vigna angularis (A0A0S3T358 and A0A0S3T2Q6), Morella rubra (A0A6A1V6U3), Camellia sinensis (A0A4S4D3Y1), Lactuca saligna (A0A6S7MYJ1), Capsicum chinense (A0A2G3C830), Citrullus lanatus (MAKER: Cla97C08G161720.1, ClCG08G018020.1 and C1CG05G022460.1). Exemplary MTOPVIB gene products from monocots include those of, by way of example, Asparagus officinalis (XP 020251573.1, XP 020251574.1 and A0A5P1FSJ9), Elaeis guineensis (XP 029123736.1), Ananas comosus (XP 020087950.1, XP 020087949.1 and A0A6P5F3Q6), Brachypodium distachyon (XP 010227454.1, XP 024313653.1 and A0A2K2DMQ9), Zea mays (XP 008645058.1, A0A3L6EMU7 and A0A1D6GVU9), Phoenix dactylifera (XP 026658358.1 and A0A3Q0HUJ8), Musa acuminata (XP 009385274.2 and M0RV08), Sorghum bicolor (XP 021304331.1, XP 021304332.1 and A0A194YLK0), Phalaenopsis equestris (XP 020572586.1), Panicum hallii (XP 025810579.1, XP 025810581.1 and A0A2S3HGH7), Setaria viridis (XP 034591320.1, A0A4U6V2N9 and A0A4U6V741), Setaria italica (XP 004967281.3, A0A368QZ11 and K3Y2U2), Oryza brachyantha (XP 015694443.1, XP 015694442.1 and J3MHH7), Oryza sativa (XP 015643072.1), Dendrobium catenatum (XP 028550772.1, A0A2I0WVS0 and A0A2I0W4A5), Triticum aestivum (A0A3B6RJU9, A0A3B6SJY5 and A0A3B6TVE9), Triticum turgidum (A0A446XR78, A0A446XRA6 and A0A446YQT1), Oryza meyeriana (A0A6G1CTE3, A0A6G1CUI2 and A0A6G1CUH8), Hordeum vulgare (F2EDE7, A0A287XFD4 and A0A287XFH2), Panicum hallii (A0A2T7DUD0 and A0A2T7DUC8), Aegilops tauschii (A0A453SMH6, A0A453SLK8 and A0A453SMK7), Oryza barthii (A0A0D3GKN6, A0A0D3GKN7 and A0A0D3GKN9), Oryza nivara (A0A0E0HV65, A0A0E0HV66 and A0A0E0HV67), Oryza sativa (B9FQU2, Q0D9M7 and A0A0P0X0S8), Panicum miliaceum (A0A3L6PDC8 and A0A3L6RZ25), Oryza sativa (B8B2K7), Triticum urartu (M7YHD9), Leersia perrieri (A0A0D9WTZ7, A0A0D9WTZ8 and A0A0D9WTZ9), Oryza rufipogon (A0A0E0Q2G4, A0A0E0Q2G1 and A0A0E0Q2G3), Eragrostis curvula (A0A5J9T8A2), Oryza glumipatula (A0A0E0AE66, A0A0E0AE67 and A0A0E0AE68), Oryza meridionalis (A0A0E0E667, A0A0E0E668 and A0A0E0E665), Oryza punctata (A0A0E0LFH0, A0A0E0LFH1 and A0A0E0LFH2), Oryza glaberrima (I1Q548), Ananas comosus (A0A6V7Q5B3), Aegilops tauschi (R7WBC9), Zostera marina (A0A0K9PK51), Musa balbisiana (A0A4V4H5P4), Dichanthelium oligosanthes (A0A1E5UYF4).

### Components of Progression Through the Second Division of Meiosis

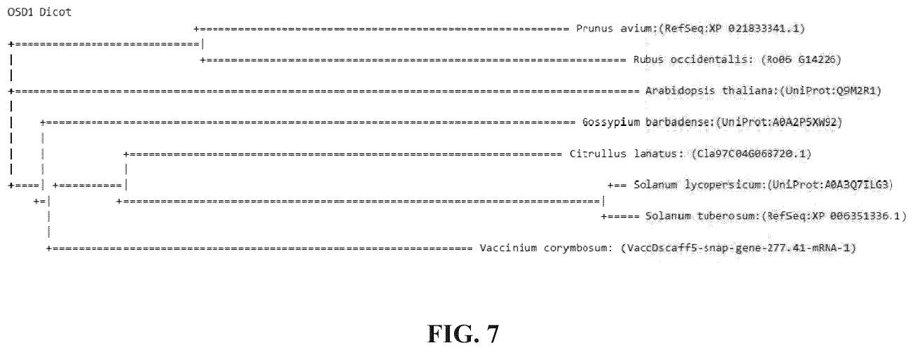

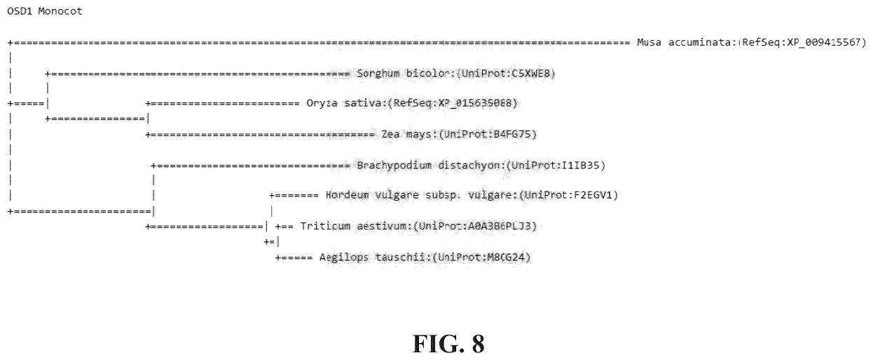

In some embodiments, the complete, partial, or partially-complemented MiMe genotype comprises one or more MiMe alleles conferring decreased expression of one or more MiMe loci of a component of progression through the second division of meiosis. Components of progression through the second division of meiosis include, for example, both (a) MiMe loci that encode gene products that are required for progression through the second division of meiosis and (b) MiMe loci that that are associated with second division restitution mechanisms, since the effective end result of both is as if the second division of meiosis did not occur, with the resulting gametes of each containing sister chromatids. Second division restitution (also known as nuclear restitution) mechanisms are known in the art and described in Brownfield and Kohler (2010. Unreduced gamete formation in plants: mechanisms and prospects. J Exp Bot 62:5, 1659-1668). In certain embodiments, the one or more MiMe loci of the component of progression through the second division of meiosis comprise OSD1, CYCA1, TDM1, PC1, PC2, FC, or any combination thereof. One of skill in the art will understand that MiMe loci of the component of progression through the second division of meiosis are not limited to OSD1, CYCA1, TDM1, PC1, PC2, and FC, and may include any loci encoding gene products required for progression through the second division of meiosis or associated with second division restitution mechanisms. By way of example only, a gene product of the component of progression through the second division of meiosis of the first division sister chromatid segregation is exemplified by an OSD1 protein (e.g., an OSD1-1 protein, an OSD1-2 protein, or an OSD1-3 protein) and specifically by the OSD1 protein sequences, sequence alignments, and percent identities described in “MiMe Gene Product Sequences” below. Representative OSD1 protein sequences from dicotyledonous plants (SEQ ID NOs: 56-64, 91, and 92) are provided in the sequence listing as outlined in Table 9, including eight native sequences and a consensus sequence identified by multiple sequence alignment of the eight native sequences (Sequence Alignment 7). Table 7A shows a matrix of percent identities of the OSD1 protein sequences from dicotyledonous plants, and a phylogenetic tree showing the relationship between the sequences is shown in FIG. 7. Representative OSD1 protein sequences from monocotyledonous plants (SEQ ID NOs: 65-75) are provided in the sequence listing as outlined in Table 9, including eight native sequences and a consensus sequence identified by multiple sequence alignment of the eight native sequences (Sequence Alignment 8), as well as OSD1-1, OSD1-2, and OSD1-3 protein sequences from maize. OSD1 homologues present in the maize genome include, for example, OSD1-1 on chromosome 2 (SEQ ID NO: 72), OSD1-2 on chromosome 5 (SEQ ID NO: 73), and OSD1-3 on chromosome 5 (SEQ ID NO: 74). Table 7B shows a matrix of percent identities of the OSD1 protein sequences from monocotyledonous plants, and a phylogenetic tree showing the relationship between the sequences is shown in FIG. 8. The gene products of MiMe loci of the component of progression through the second division of meiosis include OSD1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the OSD1 proteins of SEQ ID NOs: 56-75, 91, and 92. The gene products of MiMe loci of the component of progression through the second division of meiosis also include: a) OSD1-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the OSD1-1 protein of SEQ ID NO: 72 or SEQ ID NO: 91; b) OSD1-2 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the OSD1-2 protein of SEQ ID NO: 73 or SEQ ID NO: 92; a) OSD1-3 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the OSD1-3 protein of SEQ ID NO: 74.

There is an abundance of known OSD1 gene products from both dicotyledonous plants (dicots) and monocotyledonous plants (monocots) including all of the following, which sequences are hereby incorporated by reference in their form as of the effective filing date. Exemplary OSD1 gene products from dicots include those of, by way of example, Abrus precatorius (XP 027351083.1), Actinidia chinensis var. chinensis (A0A2R6Q4N7, A0A2R6R558, and A0A2R6S207), Arabidopsis lyrata subsp. lyrata (XP 002876442.1 and D71I35), Arabidopsis thaliana (NP 191345.1, A0A178VY10, A0A654FIR7, and Q9M2R1), Arabis nemorensis (A0A565BQ01 and A0A565BXM3), Arachis duranensis (XP 015969469.1), Arachis hypogaea (XP 025662573.1 and A0A445CLA0), Arachis ipaensis (XP 016204949.1), Beta vulgaris subsp. vulgaris (XP 010672481.1), Brassica campestris (A0A397Z630), Brassica cretica (A0A3N6RN22), Brassica napus (XP 013663545.1, XP 013739052.1, XP 013748890.1, and A0A078IUJ6), Brassica oleracea (A0A3P6C949), Brassica oleracea var. oleracea (A0A0D3D5M8 and A0A0D3DU24), Brassica rapa (XP 009116484.1 and XP 009142815.1), Cajanus cajan (XP 020213587.1), Camelina sativa (XP 010427676.1, XP 010504725.1, and XP 010516422.1), Camellia sinensis (XP 028108898.1), Camellia sinensis var. sinensis (A0A4S4EJH4), Cannabis sativa (XP 030492553.1 and XP 030492655.1), Capsicum annuum (XP 016544891.1), Capsicum baccatum (A0A2G2VVT8), Capsicum chinense (A0A2G3BH89), Carica papaya (XP 021893906.1 and XP 021893909.1), Carpinus fangiana (A0A5N6RHI3 and A0A5N6RSE0), Chenopodium quinoa (XP 021774778.1), Cicer arietinum (XP 004493551.1), Citrullus lanatus (Cla97C04G068720.1 and Cla97C04G068720.1), Citrus clementina (XP 006421493.1), Citrus sinensis (XP 015389410.1 and A0A067EQA5), Citrus unshiu (A0A2H5PTI9), Coffea arabica (XP 027066367.1), Coffea canephora (A0A068UNI9), Coffea eugenioides (XP 027176811.1), Corchorus capsularis (A0A1R3GH13), Corchorus olitorius (A0A1R3HII0), Cucumis melo (XP 008460799.1), Cucumis sativus (XP 004150162.1), Cucurbita maxima (XP 022966440.1 and XP 022967896.1), Cucurbita moschata (XP 022925136.1 and XP 022928675.1), Cucurbita pepo subsp. pepo (XP 023518760.1 and XP 023543597.1), Cuscuta australis (A0A328DWI1), Cuscuta campestris (A0A484L766), Cynara cardunculus var. scolymus (XP 024968696.1), Daucus carota subsp. sativus (XP 017216722.1, A0A161WUA0, A0A161Y439, and A0A161ZLJ1), Dorcoceras hygrometricum (A0A2Z7BPU7), Durio zibethinus (XP 022766754.1, A0A6P5YQK3, and A0A6P5Z4K9), Erythranthe guttata (XP 012857469.1), Eutrema salsugineum (XP 006402834.1 and XP 006411487.1), Fagus sylvatica (A0A2N9HHY2 and A0A2N9ISW9), Glycine soja (A0A445FGG0 and A0A445LBS7), Gossypium arboreum (XP 017628288.1), Gossypium barbadense (A0A2P5XW92 and A0A5J5PJ00), Gossypium hirsutum (XP 016708697.1 and XP 016711932.1), Gossypium mustelinum (A0A5D2XEQ5), Gossypium raimondii (XP 012434993.1), Gossypium tomentosum (A0A5D2IYP0), Helianthus annuus (XP 021970234.1, XP 022012290.1, A0A251SAA2, and A0A251UML0), Herrania umbratica (XP 021282785.1), Hibiscus syriacus (A0A6A2ZEH6, A0A6A3C7R2, and A0A6A3CDL4), Ipomoea nil (XP 019166212.1), Jatropha curcas (XP 012073041.1), Juglans regia (XP 018808537.2 and A0A2I4HQ21), Lactuca saligna (A0A6S7P8L2), Lactuca sativa (XP 023736131.1), Lupinus albus (A0A6A4P5G2 and A0A6A5P9L1), Lupinus angustifolius (XP 019418161.1 and XP 019440837.1), Malus baccata (A0A540LQN8 and A0A540NIL1), Malus domestica (A0A498I3I7 and A0A498IDC0), Manihot esculenta (XP 021597466.1 and A0A251L5J7), Medicago truncatula (XP 003625202.1), Microthlaspi erraticum (A0A6D2I7Z3 and A0A6D2KUY8), Mikania micrantha (A0A5N6NU86), Morella rubra (A0A6A1UZ72), Morus notabilis (XP 024031210.1 and W9S7X1), Nicotiana attenuata (XP 019231579.1 and XP 019252487.1), Nicotiana sylvestris (A0A1U7XAB9), Nicotiana tabacum (XP 016443699.1, XP 016486328.1, and A0A1S3ZG69), Nicotiana tomentosiformis (XP 009602908.1), Nyssa sinensis (A0A5J4ZEU2 and A0A5J4ZL33), Olea europaea var. sylvestris (XP 022842253.1), Parasponia andersonii (A0A2P5CAS1), Phaseolus vulgaris (XP 007162280.1), Pistacia vera (XP 031280295.1, XP 031282986.1, and XP 031287164.1), Populus alba (XP 034893325.1 and XP 034918809.1), Populus euphratica (XP 011020285.1 and XP 011033266.1), Populus trichocarpa (XP 002323297.1 and XP 006381043.1), Prunus avium (XP 021833341.1), Prunus dulcis (XP 034226293.1), Prunus mume (XP 008240815.1), Prunus persica (XP 020424060.1 and M5WA56), Prunus yedoensis var. nudiflora (A0A314UC93 and A0A314YZQ1), Punica granatum (A0A218W3I8), Pyrus ussuriensis x Pyrus communis (A0A5N5G3Z9 and A0A5N5HYZ1), Pyrus x bretschneideri (XP 009345103.1 and XP 009347481.1), Quercus lobata (XP 030925248.1), Quercus suber (XP 023892570.1, XP 023903653.1, and XP 023903669.1), Raphanus sativus (XP 018436735.1, XP 018462483.1, and A0A6J0NRE3), Ricinus communis (B9T2D4), Rubus occidentalis (Ro06_G14226), Salix brachista (A0A5N5JJP5 and A0A5N5MC65), Salix viminalis (A0A6N2K1T3 and A0A6N2NJF8), Salvia splendens (A0A4D8XYD7, A0A4D9ACL9, and A0A4D9B0E0), Sesamum indicum (XP 011078856.1), Solanum chacoense (A0A0V0HQ03), Solanum chilense (A0A6N2B6M6), Solanum lycopersicum (A0A3Q7ILG3), Solanum pennellii (XP 015089255.1), Solanum tuberosum (XP 006351336.1), Spinacia oleracea (A0A0K9R7P1), Striga asiatica (A0A5A7R3X1), Tarenaya hassleriana (XP 010524304.1 and XP 010554851.1), Theobroma cacao (XP 007023264.2, XP 007028924.1, and A0A061G8H9), Trema orientale (A0A2P5F1H9), Trifolium pratense (A0A2K3N2Q1), Vaccinium corymbosum (MAKER: VaccDscaff5-snap-gene-277.41-mRNA-1, VaccDscaff10-augustus-gene-132.35-mRNA-1, and VaccDscaff8-augustus-gene-151.22-mRNA-1), Vigna angularis (XP 017417753.1), Vigna radiata var. radiata (XP 014495445.1), Vigna unguiculata (XP 027940039.1), Vitis riparia (XP 034693215.1), Vitis vinifera (XP 002277253.1 and A0A438EGU9), and Ziziphus jujuba (A0A6P6G4E6). Exemplary OSD1 gene products from monocots include those of, by way of example, Aegilops tauschii (M8CG24), Aegilops tauschii subsp. strangulata (A0A453NZH5), Aegilops tauschii subsp. tauschii (XP 020185497.1), Brachypodium distachyon (A0A0Q3IAH2 and I1IB35), Dichanthelium oligosanthes (A0A1E5WI68), Eragrostis curvula (A0A5J9TWQ6, A0A5J9V0F4, A0A5J9V0Q5, A0A5J9V1T7, and A0A5J9V3P7), Hordeum vulgare subsp. vulgare (A0A287U6J0, F2EGV1, and M0USV7), Leersia perrieri (A0A0D9VHQ1 and A0A0D9W656), Musa acuminata subsp. malaccensis (XP 009401510.1), Musa balbisiana (A0A4S8JYK8 and A0A4S8K685), Oryza barthii (A0A0D3FWY7), Oryza brachyantha (J3LEB7, J3LYU0, and I1PM63), Oryza glaberrima (I1PM63), Oryza glumipatula (A0A0D9ZM14), Oryza meridionalis (A0A0E0CMP6), Oryza meyeriana var. granulata (A0A6G1BR28), Oryza nivara (A0A0E0G954), Oryza punctata (A0A0E0K1S9 and A0A0E0KRS9), Oryza rufipogon (A0A0E0NH48 and A0A0E0PAE9), Oryza sativa subsp. indica (A2XUJ4), Oryza sativa subsp. japonica (A0A0P0WBP1, B7F906, and Q0JCG0), Panicum hallii (XP 025825740.1, A0A2S3GQS8, and A0A2T8ID88), Panicum hallii var. hallii (A0A2T7CWA3, A0A2T7F8B5, and A0A2T7F8D4), Panicum miliaceum (A0A3L6PTY0, A0A3L6Q0C4, A0A3L6Q7A4, A0A3L6QKY9, and A0A3L6RS82), Setaria italica (A0A368PPY6, K3Y9S9, K3YVP1, and K3YVU1), Setaria viridis (A0A4U6WFM9), Sorghum bicolor (A0A1B6PLK1, C5XWE8, and C5XWF1), Triticum aestivum (A0A3B6C8J9 and A0A3B6PLJ3), Triticum turgidum subsp. durum (A0A446L6V2, A0A446MFQ1, and A0A446WCJ4), Triticum urartu (M7YNE4), and Zea mays (A0A1D6HBV9, A0A1D6HBW0, B4FCP3, B4FG75, and B6T7U1).

By way of example only, a gene product of the component of progression through the second division of meiosis is also exemplified by a CYCA1 protein, also known as CYCLIN-A1 or TARDY ASYNCHRONOUS MEIOSIS (TAM), and specifically by the CYCA1 protein sequences, sequence alignments, and percent identities described in “MiMe Gene Product Sequences” below. Representative CYCA1 protein sequences from dicotyledonous plants (SEQ ID NOs: 81-83, 96-104, and 93) are provided in the sequence listing as outlined in Table 9, including native sequences and a consensus sequence identified by multiple sequence alignment of the native sequences (Sequence Alignment 10). Table 8B shows a matrix of percent identities of the CYCA1 protein sequences from dicotyledonous plants. Representative CYCA1 protein sequences from monocotyledonous plants (SEQ ID NOs: 78 and 105-119) are provided in the sequence listing as outlined in Table 9, including native sequences and a consensus sequence identified by multiple sequence alignment of the native sequences (Sequence Alignment 9). Table 8A shows a matrix of percent identities of the CYCA1 protein sequences from monocotyledonous plants. The gene products of MiMe loci of the component of progression through the second division of meiosis include CYCA1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the CYCA1 proteins of SEQ ID NOs: 78, 81-83, 96-119, and 93.

There is an abundance of known CYCA1 gene products from both dicotyledonous plants (dicots) and monocotyledonous plants (monocots) including all of the following, which sequences are hereby incorporated by reference in their form as of the effective filing date. Exemplary CYCA1 gene products from dicots include those of, by way of example, Rubus occidentalis (MAKER: Ro02_G01483, Ro02_G14787 and Ro02_G00540), Vaccinium corymbosum (MAKER: VaccDscaff3-augustus-gene-243.30-mRNA-1, VaccDscaff25-augustus-gene-211.25-mRNA-1 and VaccDscaff2-augustus-gene-199.26-mRNA-1), Eutrema salsugineum (XP 006390095.1, XP 024006188.1 and XP 006393746.1), Camelina sativa (XP 010471932.1, XP 010416680.1 and XP 010429845.1), Arabidopsis lyrata (XP 002889141.1, XP 002893979.1 and XP 002890110.1), Arabidopsis thaliana (NP 177863.2, NP_175077.1 and NP_173010.1), Brassica oleracea (XP 013591664.1, XP 013591665.1 and XP 013616674.1), Raphanus sativus (XP 018455173.1, XP 018447783.1 and XP 018487235.1), Brassica rapa (XP 009106401.1, XP 009145051.1 and XP 009126172.1), Hevea brasiliensis (XP 021658850.1, XP 021667309.1 and A0A6A6L6S7), Brassica napus (XP 013720220.1, XP 013652488.1 and XP 013703985.1), Tarenaya hassleriana (XP 010521286.1, XP 010521285.1 and XP 010532201.1), Carica papaya (XP 021896341.1 and XP 021893528.1), Populus alba (XP 034927970.1, XP 034889058.1 and A0A4U5R6W9), Manihot esculenta (XP 021603578.1, XP 021600594.1 and A0A2C9WH73), Populus trichocarpa (XP 006386358.1, XP 002306649.1 and XP 006371726.2), Citrus sinensis (XP 006472826.1, XP 006469769.1 and XP 024950590.1), Ricinus communis (XP 015575360.1 and B9S2W0), Populus euphratica (XP 011003420.1, XP 011026520.1 and XP 011026521.1), Vitis vinifera (XP 002284567.1, XP 010648552.1 and XP 019074759.1), Juglans regia (XP 018855785.2, XP 035541889.1 and XP 018844710.1), Vitis riparia (XP 034675471.1, XP 034676795.1 and XP 034676794.1), Durio zibethinus (XP 022766336.1, XP 022753455.1 and XP 022772335.1), Ziziphus jujuba (XP 015885638.1, XP 015885640.1 and XP 024930295.1), Herrania umbratica (XP 021284517.1, XP 021284735.1 and XP 021284369.1), Sesamum indicum (XP 011102309.1, XP 011087041.1 and XP 011081889.1), Morus notabilis (XP 010095430.1, XP 010095429.1 and XP 010110307.1), Prunus avium (XP 021806552.1, XP 021825278.1 and A0A6P5RVD6), Camellia sinensis (XP 028058625.1, XP 028087752.1 and XP 028079326.1), Theobroma cacao (XP 007019157.2, XP 007019153.2 and XP 007013748.2), Lupinus angustifolius (XP 019454427.1, XP 019417838.1 and XP 019414882.1), Prunus dulcis (XP 034229204.1, XP 034197022.1 and A0A5E4ECX5), Citrus clementina (XP 006434258.1, XP 006434257.2 and V4SIF7), Quercus lobata (XP 030933779.1, XP 030933780.1 and XP 030933781.1), Prunus persica (XP 007225619.1, XP 020415692.1 and M5XYD4), Pistacia vera (XP 031273143.1, XP 031273144.1 and XP 031254933.1), Punica granatum (XP 031389984.1, XP 031389873.1 and A0A218XKA7), Rosa chinensis (XP 024166503.1, XP 024165607.1 and XP 024166732.1), Prunus mume (XP 008219598.1, XP 008219599.1 and XP 008219856.1), Fragaria vesca (XP 004290902.1, XP 004292715.1 and XP 004289008.1), Malus domestica (XP 028950632.1, XP 028950835.1 and XP 028954464.1), Cucurbita moschata (XP 022938814.1, XP 022938813.1 and XP 022953995.1), Gossypium arboreum (XP 017620373.1, XP 017639312.1 and XP 017645154.1), Cucurbita maxima (XP 022993869.1, XP 022991147.1 and A0A6J1K1C6), Eucalyptus grandis (XP 010063417.2, XP 010061967.1 and XP 010061968.1), Quercus suber (XP 023916522.1), Cucurbita pepo (XP 023550099.1, XP 023550098.1 and XP 023542826.1), Rhodamnia argentea (XP 030527169.1, XP 030527160.1 and XP 030543155.1), Gossypium raimondii (XP 012465382.1, XP 012446221.1 and XP 012446220.1), Pyrus x (XP 009343979.1, XP 018507607.1 and XP 018506600.1), Coffea arabica (XP 027091321.1, XP 027094124.1 and XP 027091322.1), Helianthus annuus (XP 022038561.1 and A0A251UQE6), Gossypium hirsutum (XP 016742640.1, XP 016729480.1 and A0A1U8NUW1), Glycine soja (XP 028235914.1, XP 028196463.1 and XP 028199264.1), Momordica charantia (XP 022141694.1, XP 022154953.1 and A0A6J1CJH3), Phaseolus vulgaris (XP 007137455.1, XP 007161469.1 and V7AV33), Cucumis sativus (XP 004149427.1 and A0A0A0KDS0), Abrus precatorius (XP 027339521.1, XP 027368331.1 and XP 027339131.1), Spinacia oleracea (XP 021839596.1 and A0A0K9QD06), Cucumis melo (XP 008466750.1 and A0A1S3CS10), Cajanus cajan (XP 020206943.1, XP 020212690.1 and XP 029127066.1), Vigna unguiculata (XP 027939695.1, XP 027902093.1 and XP 027902094.1), Erythranthe guttata (XP 012843290.1, XP 012855865.1 and A0A022QXA3), Cynara cardunculus (XP 024976951.1 and A0A103XZ63), Vigna angularis (XP 017420796.1 and XP 017429427.1), Ipomoea nil (XP 019196844.1, XP 019196846.1 and XP 019173581.1), Cicer arietinum (XP 012571947.1, XP 027190936.1 and A0A1S3E7Z2), Nicotiana tabacum (XP 016472550.1, NP_001312515.1 and XP 016435631.1), Lactuca sativa (XP 023747498.1 and A0A2J6M9K9), Ipomoea triloba (XP 031119192.1), Nicotiana tomentosiformis (XP 009593177.1, XP 009593178.1 and XP 018624925.1), Arachis hypogaea (XP 025612901.1, XP 025665575.1 and XP 025665574.1), Arachis duranensis (XP 020984390.1, XP 020984389.1 and XP 020984391.1), Vigna radiata (XP 014498669.1, XP 014503828.1 and XP 014503829.1), Arachis ipaensis (XP 016163479.1, XP 016163477.1 and XP 016163478.1), Medicago truncatula (XP 013461288.1, XP 013461287.1 and A0A072V1U7), Syzygium oleosum (XP 030444327.1, XP 030444328.1 and XP 030452807.1), Beta vulgaris (XP 010684698.1), Daucus carota (XP 017252769.1, XP 017253032.1 and XP 017243271.1), Nicotiana attenuata (XP 019245556.1, XP 019235783.1 and A0A1J6J033), Solanum tuberosum (XP 006351137.1, XP 006351136.1 and XP 006351138.1), Capsicum annuum (XP 016569717.1, XP 016569718.1 and XP 016569719.1), Olea europaea (XP 022875899.1, XP 022867393.1 and XP 022879684.1), Glycine max (NP_001241939.1, K7KUJ6 and Q39879), Cannabis sativa (XP 030509576.1, XP 030488433.1 and XP 030488434.1), Chenopodium quinoa (XP 021717819.1), Solanum pennellii (XP 015058346.1 and XP 015058345.1), Solanum lycopersicum (NP_001303847.1, NP_001233762.1 and Q9XGI5), Coffea eugenioides (XP 027181508.1), Nicotiana sylvestris (XP 009758618.1 and A0A1U7Y359), Arabis nemorensis (A0A565BIS1 and A0A565CDD7), Microthlaspi erraticum (A0A6D2HEW0 and A0A6D2I8E8), Brassica oleracea (A0A3P6GGQ8, A0A3P6CST8 and A0A3P6CYA6), Brassica campestris (A0A3P6BUE1, A0A397YUI1 and A0A397ZET1), Arabis alpina (A0A087HJ31, A0A087G2V9 and A0A087H7V5), Brassica rapa (M4DGP6, M4ETS0 and M4E4A3), Jatropha curcas (A0A067KCU7 and A0A067KKF4), Trema orientale (A0A2P5BNJ4, A0A2P5BNI8 and A0A2P5G255), Citrus unshiu (A0A2H5PIC6, A0A2H5PID6 and A0A2H5Q6J9), Parasponia andersonii (A0A2P5BF06, A0A2P5BF17 and A0A2P5CAE5), Salix brachista (A0A5N5MQK8 and A0A5N5NN32), Salix viminalis (A0A6N2MHL6 and A0A6N2M526), Actinidia chinensis (A0A2R6R3V3, A0A2R6QYR8 and A0A2R6Q1J6), Corchorus capsularis (A0A1R3FX40, A0A1R3FX22 and A0A1R3G2T5), Acer yangbiense (A0A5C7H7L6 and A0A5C7HYW9), Prunus armeniaca (A0A6J5WGG6, A0A6J5TUK7 and A0A6J5TWS8), Nyssa sinensis (A0A5J5BTV0 and A0A5J5BUP5), Pyrus ussuriensis (A0A5N5GA12, A0A5N5G9M5 and A0A5N5HSG7), Carpinus fangiana (A0A5N6RKY6 and A0A5N6RME8), Malus baccata (A0A540MN67, A0A540LX60 and A0A540MLJ9), Lupinus albus (A0A6A4PVU1, A0A6A5NWS9 and A0A6A4PQN4), Gossypium barbadense (A0A5J5WMJ8, A0A2P5VPW8 and A0A5J5VU41), Gossypium darwinii (A0A5D2HD77, A0A5D2GJW0 and A0A5D2CM85), Gossypium mustelinum (A0A5D3A4H4, A0A5D2ZCG0 and A0A5D2V0R5), Gossypium tomentosum (A0A5D2RGV8, A0A5D2L0B6 and A0A5D2QJE2), Gossypium australe (A0A5B6UHA4, A0A5B6UJ82 and A0A5B6W535), Coffea canephora (A0A068URS1 and A0A068VFE6), Morella rubra (A0A6A1WAG0 and A0A6A1W9N1), Corchorus olitorius (A0A1R3GAC4 and A0A1R3ID39), Cephalotus follicularis (A0A1Q3BVV0 and A0A1Q3BVK1), Cucumis melo (A0A5D3CF82 and A0A5A7UPU9), Striga asiatica (A0A5A7QVQ6), Prunus yedoensis (A0A314YEC6), Dorcoceras hygrometricum (A0A2Z7CXN3), Vigna angularis (A0A0S3RDJ0 and A0A0S3RNB9), Lactuca saligna (A0A6S7P686), Trifolium pratense (A0A2K3P0W8), Salvia splendens (A0A4D8XZ56, A0A4D9BYQ7 and A0A4D8YRC7), Fagus sylvatica (A0A2N9FDV3 and A0A2N9FCX3), Capsicum chinense (A0A2G3AYR4), Phaseolus angularis (A0A0L9V0A2 and A0A0L9UG75), Capsicum baccatum (A0A2G2WYF3), Cuscuta campestris (A0A484NDE4 and A0A484M2X3), Solanum chilense (A0A6N2BZS6), Mikania micrantha (A0A5N6N619), Cuscuta australis (A0A328CZK8), Hibiscus syriacus (A0A6A3BH35, A0A6A2XWY9 and A0A6A2XR37), Camellia sinensis (A0A4S4EQV7, A0A4S4DSV8 and A0A4S4E291), Citrullus lanatus (MAKER: Cla97C09G164540.1, Cla97C08G148690.1 and Cla97C08G156180.1). Exemplary CYCA1 gene products from monocots include those of, by way of example, Oryza sativa (XP 015630726.1, XP 025879970.1 and XP 025878069.1), Oryza brachyantha (XP 015699234.1, XP 015688228.1 and XP 006654172.1), Brachypodium distachyon (XP 003566077.1, XP 003566081.1 and XP 003566404.1), Aegilops tauschi (XP 020187359.1), Sorghum bicolor (XP 002455447.1, XP 002454880.1 and XP 021311446.1), Panicum hallii (XP 025814057.1, XP 025808556.1 and XP 025808555.1), Zea mays (NP_001105387.2, NP_001288521.1 and XP 008656316.2), Setaria viridis (XP 034584257.1, XP 034584256.1 and XP 034584258.1), Ananas comosus (XP 020097777.1, A0A6P5FQ19 and A0A199VUU2), Setaria italica (XP 004962645.1, XP 022680676.1 and XP 022680677.1), Phoenix dactylifera (XP 008783212.1, XP 008806186.1 and XP 017701045.1), Elaeis guineensis (XP 010915931.1, XP 010925928.1 and XP 019707175.1), Musa acuminata (XP 009387253.1, XP 018678008.1 and XP 018681842.1), Phalaenopsis equestris (XP 020589936.1), Dendrobium catenatum (XP 020688416.1 and A0A2I0XHK3), Asparagus officinalis (XP 020270124.1, XP 020246640.1 and XP 020246630.1), Oryza sativa (Q7F830, A0A0P0V089 and Q0INT0), Oryza sativa (B8AB18, B8AB17 and B8AZU7), Oryza glumipatula (A0A0D9Y5P8, A0A0D9Y5P6 and A0A0D9ZW29), Oryza meridionalis (A0A0E0BZK6, A0A0E0BZK3 and A0A0E0BZK4), Oryza barthi (A0A0D3ELF6 and A0A0D3G4V0), Oryza meyeriana (A0A6G1DY77), Oryza punctata (A0A0E0JG18 and A0A0E0L12), Leersia perrieri (A0A0D9UYN6, A0A0D9UYN4 and A0A0D9UYN5), Oryza rufipogon (A0A0E0MTK1, A0A0E0PJC5 and A0A0E0PJC4), Oryza nivara (A0A0E0FJ42 and A0A0E0HBD1), Hordeum vulgare (F2DI11), Triticum turgidum (A0A446NBG5, A0A446PKW3 and A0A446ISF2), Triticum turgidum (A0A096XWV9 and A0A096XWV8), Triticum aestivum (A0A3B6EEN6, A0A096XWW5 and A0A077RZA4), Eragrostis curvula (A0A5J9UII2, A0A5J9UGY6 and A0A5J9VDW9), Aegilops tauschi (A0A453EIL2, A0A453EIL3 and A0A453EIL1), Panicum hallii (A0A2T7DTF4, A0A2T7EGY6 and A0A2T7EGY2), Panicum miliaceum (A0A3L6SV79, A0A3L6RBV3 and A0A3L6T2B6), Dichanthelium oligosanthes (A0A1E5US47, A0A1E5VMF4 and A0A1E5WHF7), Triticum urartu (M7YGF0), Elaeis guineensis (A0A6I9QX63, A0A6I9RNP0 and A0A6J0PKS8), Musa balbisiana (A0A4V4H2T6 and A0A4S8JV86), Ananas comosus (A0A6V7QK40), Oryza glaberrima (I1PTL4), Zostera marina (A0A0K9NY27 and A0A0K9P0G3).

By way of example only, a gene product of the component of progression through the second division of meiosis is also exemplified by a TDM1 protein, and specifically by the TDM1 protein sequences and percent identities described herein. Representative TDM1 protein sequences from dicotyledonous plants (SEQ ID NOs: 84, 94, and 95) are provided in the sequence listing as outlined in Table 9, including TDM1-1 (SEQ ID NO: 94) and TDM1-2 (SEQ ID NO: 95) protein sequences from soybean. A representative TDM1 protein sequence from a monocotyledonous plant (SEQ ID NO: 79) is provided in Table 9. The gene products of MiMe loci of the component of progression through the second division of meiosis include TDM1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to any one of the TDM1 proteins of SEQ ID NOs: 79, 84, 94, and 95. The gene products of MiMe loci of the component of progression through the second division of meiosis also include: a) TDM1-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the protein of SEQ ID NO: 94; and b) TDM1-2 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the protein of SEQ ID NO: 95.

There is an abundance of known TDM1 gene products from both dicotyledonous plants (dicots) and monocotyledonous plants (monocots) including all of the following, which sequences are hereby incorporated by reference in their form as of the effective filing date. Exemplary TDM1 gene products from dicots include those of, by way of example, Rubus occidentalis (MAKER: Ro03_G16141, Ro03_G15791 and Ro01_G29637), Vaccinium corymbosum (MAKER: VaccDscaff44-augustus-gene-134.17-mRNA-1, VaccDscaff28-augustus-gene-132.24-mRNA-1 and VaccDscaff20-augustus-gene-221.16-mRNA-1), Camelina sativa (XP 010439553.1, XP 010449143.1 and XP 010434260.1), Eutrema salsugineum (XP 024004526.1, XP 006403107.1 and XP 006403934.1), Arabidopsis thaliana (NP_001328331.1, NP_193822.1 and NP_199246.1), Arabidopsis lyrata (XP 002867870.1, XP 002865370.1 and XP 020879933.1), Tarenaya hassleriana (XP 010537572.1, XP 010550475.1 and XP 010558830.1), Brassica oleracea (XP 013598365.1, XP 013586912.1 and XP 013586904.1), Brassica rapa (XP 018512518.2, XP 009101720.1 and XP 009136610.1), Raphanus sativus (XP 018479192.1, XP 018434569.1 and XP 018455102.1), Brassica napus (XP 013737533.1, XP 013717465.1 and XP 022563007.1), Manihot esculenta (XP 021634691.1, XP 021604050.1 and XP 021617885.1), Jatropha curcas (XP 012075600.1, XP 012088514.1 and XP 012092647.1), Quercus lobata (XP 030967932.1, XP 030974498.1 and XP 030967394.1), Hevea brasiliensis (XP 021661292.1, XP 021679065.1 and XP 021655186.1), Carica papaya (XP 021888497.1, XP 021908124.1 and XP 021907366.1), Quercus suber (XP 023891553.1, XP 023924030.1 and XP 023915038.1), Populus trichocarpa (XP 002300564.2, XP 024436721.1 and XP 006377952.1), Theobroma cacao (XP 007022916.2, XP 007046648.2 and XP 007034430.2), Gossypium hirsutum (XP 016688775.1, XP 016730194.1 and XP 016696659.1), Herrania umbratica (XP 021298259.1, XP 021300673.1 and XP 021290143.1), Gossypium arboreum (XP 017606464.1, XP 017627676.1 and A0A6P4NIJ0), Populus euphratica (XP 011037742.1, XP 011037741.1 and XP 011037740.1), Gossypium raimondii (XP 012444753.1, XP 012491503.1 and XP 012490805.1), Juglans regia (XP 018844091.2, XP 018809026.1 and XP 018829559.1), Populus alba (XP 034889284.1, XP 034890557.1 and XP 034889701.1), Nicotiana tabacum (XP 016501712.1, XP 016448629.1 and XP 016503322.1), Erythranthe guttata (XP 012827439.1, XP 012845130.1 and XP 012828928.1), Eucalyptus grandis (XP 018721991.1, XP 010036611.1 and XP 010036609.1), Nicotiana tomentosiformis (XP 009624596.1, XP 009621343.1 and XP 009617544.1), Cicer arietinum (XP 004486472.1, XP 004509379.1 and XP 004492325.1), Camellia sinensis (XP 028052208.1, XP 028084107.1 and XP 028125201.1), Phaseolus vulgaris (XP 007147465.1, XP 007156178.1 and XP 007140568.1), Abrus precatorius (XP 027338252.1, XP 027347849.1 and XP 027353149.1), Nicotiana sylvestris (XP 009780563.1, XP 009796805.1 and XP 009793488.1), Citrus sinensis (XP 006491596.1, XP 024950529.1 and XP 006466984.1), Cannabis sativa (XP 030479622.1, XP 030479621.1 and XP 030479623.1), Prunus avium (XP 021816234.1, XP 021816320.1 and XP 021807023.1), Spinacia oleracea (XP 021849395.1, XP 021851108.1 and XP 021849117.1), Morus notabilis (XP 024022818.1, XP 010113327.1 and XP 010109708.1), Pyrus x (XP 009362298.1, XP 009379370.1 and XP 009363476.1), Vigna angularis (XP 017435386.1, XP 017425185.1 and XP 017417852.1), Helianthus annuus (XP 022023242.1, XP 022015884.1 and XP 022012324.1), Cucurbita maxima (XP 023001024.1, XP 022994750.1 and XP 022977428.1), Citrus clementina (XP 006421192.1, XP 006448878.1 and XP 006425415.1), Cajanus cajan (XP 020230798.1, XP 020230797.1 and XP 020203814.1), Vigna unguiculata (XP 027931542.1, XP 027917075.1 and XP 027926501.1), Prunus dulcis (XP 034212275.1, XP 034226353.1 and XP 034228273.1), Fragaria vesca (XP 004295599.1, XP 004289081.1 and XP 011458200.1), Prunus persica (XP 007214224.1, XP 007201977.1 and XP 007201841.1), Cucurbita moschata (XP 022927078.1, XP 022954417.1 and XP 022949703.1), Rosa chinensis (XP 024159267.1, XP 024177572.1 and XP 024161932.1), Vigna radiata (XP 014517768.1, XP 014507152.1 and XP 014489588.1), Pistacia vera (XP 031249250.1, XP 031263948.1 and XP 031278270.1), Sesamum indicum (XP 011096694.1, XP 011099872.1 and XP 011080175.1), Coffea eugenioides (XP 027185715.1, XP 027179178.1 and XP 027178156.1), Prunus mume (XP 016648173.1, XP 008242054.1 and XP 008235504.1), Cucumis melo (XP 008445439.1, XP 008445490.1 and XP 008440837.1), Coffea arabica (XP 027086295.1, XP 027088290.1 and A0A6P6U6G3), Lactuca sativa (XP 023734516.1, XP 023749412.1 and A0A2J6KDU3), Momordica charantia (XP 022139858.1, XP 022132810.1 and XP 022146883.1), Syzygium oleosum (XP 030464735.1, XP 030464733.1 and XP 030464101.1), Cucumis sativus (XP 011659062.1, XP 011659066.1 and XP 004134853.1), Medicago truncatula (XP 003594552.1, XP 003629273.1 and XP 013448793.2), Nicotiana attenuata (XP 019253455.1, XP 019227194.1 and XP 019239871.1), Ipomoea nil (XP 019173382.1, XP 019182709.1 and XP 019172059.1), Cynara cardunculus (XP 024990731.1, XP 024994843.1 and XP 024973630.1), Chenopodium quinoa (XP 021763774.1, XP 021727595.1 and XP 021759660.1), Malus domestica (XP 008383466.2, XP 008372250.1 and XP 008337995.2), Cucurbita pepo (XP 023520226.1, XP 023542327.1 and XP 023545018.1), Arachis ipaensis (XP 016197744.2, XP 016197745.2 and XP 016196602.1), Beta vulgaris (XP 010687076.1, XP 010679906.1 and XP 010684938.1), Arachis hypogaea (XP 025646278.1, XP 025694320.1 and XP 025647132.1), Ipomoea triloba (XP 031117368.1, XP 031096079.1 and XP 031108805.1), Solanum pennellii (XP 015077927.1, XP 015063555.1 and XP 015088743.1), Capsicum annuum (XP 016577799.1, XP 016560484.1 and XP 016573624.1), Solanum lycopersicum (XP 004242080.1, XP 004233743.1 and XP 004247838.1), Vitis vinifera (XP 002266141.2, XP 002265165.1 and XP 010648997.1), Arachis duranensis (XP 015959295.2, A0A6P4D0M7 and A0A6P4CHQ1), Solanum tuberosum (XP 006350746.1, XP 006340757.1 and XP 006360343.1), Vitis riparia (XP 034697547.1, XP 034682678.1 and XP 034691907.1), Ziziphus jujuba (XP 015889748.1, XP 015892312.1 and A0A6P4A618), Punica granatum (XP 031389198.1, XP 031373547.1 and A0A218W135), Ricinus communis (XP 002529643.1, XP 015577246.1 and XP 025013464.1), Durio zibethinus (XP 022775435.1, XP 022739728.1 and XP 022758626.1), Daucus carota (XP 017238813.1, XP 017239165.1 and XP 017217353.1), Olea europaea (XP 022855716.1, XP 022860597.1 and XP 022865011.1), Glycine soja (XP 028205688.1, XP 028212169.1 and A0A0B2QNJ3), Lupinus angustifolius (XP 019448705.1, XP 019434492.1 and XP 019450500.1), Glycine max (XP 003547919.1, XP 003552145.1 and K7LBG7), Rhodamnia argentea (XP 030532808.1), Arabis nemorensis (A0A565CFG4 and A0A565C2L1), Brassica oleracea (A0A3P6EVE4, A0A3P6EW76 and A0A3P6FDS6), Brassica campestris (A0A397KV52, A0A3P5ZSC6 and A0A3P5Z7J3), Arabis alpina (A0A087GFN3, A0A087GP43 and A0A087GWW7), Brassica rapa (M4FCH5, M4EY45 and M4D8P9), Microthlaspi erraticum (A0A6D2KQE3, A0A6D2HN33 and A0A6D2HXK3), Hibiscus syriacus (A0A6A2XFQ3, A0A6A2WHY5 and A0A6A2X4H2), Salix viminalis (A0A6N2LIQ4, A0A6N2LRY7 and A0A6N2LFQ2), Gossypium barbadense (A0A5J5RCD8, A0A5J5RGF9 and A0A2P5XWC8), Gossypium darwinii (A0A5D2CIM4, A0A5D2GE99 and A0A5D2EMV1), Gossypium mustelinum (A0A5D2UVV0, A0A5D2Z843 and A0A5D2X7R3), Gossypium australe (A0A5B6X905, A0A5B6VZH9 and A0A5B6VZX1), Gossypium tomentosum (A0A5D2QDW8, A0A5D2KU73 and A0A5D2NDG2), Acer yangbiense (A0A5C7ILX7, A0A5C7HU42 and A0A5C7I416), Actinidia chinensis (A0A2R6PYE1, A0A2R6Q238 and A0A2R6RIY4), Nyssa sinensis (A0A5J4ZKL3 and A0A5J5ADZ7), Trema orientale (A0A2P5FT98, A0A2P5EUY9 and A0A2P5CWB3), Striga asiatica (A0A5A7NWH6 and A0A5A7QT27), Parasponia andersonii (A0A2P5AEY1, A0A2P5BXE6 and A0A2P5BT51), Fagus sylvatica (A0A2N9JAW3 and A0A2N9J3Q2), Cephalotus follicularis (A0A1Q3BCU0, A0A1Q3BA19 and A0A1Q3BRN8), Salix brachista (A0A5N5JRN6, A0A5N5KXB2 and A0A5N5MRG1), Phaseolus angularis (A0A0L9VDL5, A0A0L9TP14 and A0A0L9U5B0), Vigna angularis (A0A0S3S4H7, A0A0S3R4C3 and A0A0S3RUV6), Prunus yedoensis (A0A314ZWB6, A0A314Y0D8 and A0A314U6H2), Camellia sinensis (A0A4S4F0L4 and A0A4S4DX22), Mikania micrantha (A0A5N6NND9), Cuscuta campestris (A0A484LZ14, A0A484LDL3 and A0A484MGT3), Dorcoceras hygrometricum (A0A2Z7C4S6), Prunus armeniaca (A0A6J5X778, A0A6J5UKE9 and A0A6J5Y1A5), Salvia splendens (A0A4D8Y364, A0A4D8YXA1 and A0A4D9AMI9), Trifolium pratense (A0A2K3PQS3, A0A2K3NSC1 and A0A2K3N5H0), Coffea canephora (A0A068UVJ7 and A0A068U2N6), Cucumis melo (A0A5A7V6P1, A0A5A7VD19 and A0A5D3C2B5), Citrus unshiu (A0A2H5Q868 and A0A2H5N1W7), Carpinus fangiana (A0A5N6RGZ3, A0A5N6QTZ1 and A0A5N6QHL1), Capsicum baccatum (A0A2G2WNH6 and A0A2G2XG42), Malus baccata (A0A540KMB4, A0A540NLZ8 and A0A540LGX5), Lupinus albus (A0A6A5NGZ9, A0A6A4QT97 and A0A6A5PG46), Capsicum chinense (A0A2G3CB18 and A0A2G3D7L3), Pyrus ussuriensis (A0A5N5IQJ5, A0A5N5GSB3 and A0A5N5FLA7), Lactuca saligna (A0A6S7MKU2 and A0A6S7MQA7), Solanum chilense (A0A6N2B1L5 and A0A6N2BWF0), Corchorus olitorius (A0A1R3JV69 and A0A1R3ILS8), Cuscuta australis (A0A328DN36 and A0A328DLZ1), Morella rubra (A0A6A1VL61, A0A6A1WKY4 and A0A6A1UNE9), Trifolium subterraneum (A0A2Z6NRI5), Solanum chacoense (A0A0V0I6U1), Brassica cretica (A0A3N6PUR2 and A0A3N6SCF4), Populus trichocarpa (A9PJG6), Citrullus lanatus (Cla97C05G084150.1, Cla97C03G059510.1 and Cla97C07G130130.1). Exemplary TDM1 gene products from monocots include those of, by way of example, Oryza sativa (XP 015612021.2 and XP 015637899.1), Panicum hallii (XP 025803175.1, XP 025807892.1 and A0A2S3H249), Oryza brachyantha (XP 006660901.1, XP 006654613.2 and J3MZL3), Brachypodium distachyon (XP 003578528.1, XP 003568155.1 and I1ISA1), Sorghum bicolor (XP 002460580.1, XP 021308446.1 and XP 002440034.1), Zea mays (NP_001170740.1, NP_001141607.1 and XP 008651103.1), Aegilops tauschii (XP 020194824.1, XP 020194823.1 and XP 020182700.1), Elaeis guineensis (XP 010935102.1, XP 029124435.1 and XP 010941312.1), Ananas comosus (XP 020079818.1, XP 020091464.1 and A0A199V3E5), Musa acuminata (XP 018678844.1, XP 009388456.1 and XP 009409232.1), Asparagus officinalis (XP 020243978.1 and A0A5P1FNX2), Phoenix dactylifera (XP 017701542.1, XP 017701500.1 and XP 008808718.1), Phalaenopsis equestris (XP 020575044.1, XP 020573980.1 and XP 020586466.1), Dendrobium catenatum (XP 020694238.1, XP 020700034.1 and XP 020673255.1), Setaria italica (XP 004961585.1 and K3ZSM2), Setaria viridis (XP 034584403.1 and A0A4U6W9K9), Oryza rufipogon (A0A0E0QUF7), Oryza sativa (Q0J004 and Q69JE7), Oryza nivara (A0A0E0IN01 and A0A0E0HGJ7), Oryza glaberrima (I1QQT5 and I1PX27), Oryza sativa (A2Z3J2), Oryza meridionalis (A0A0E0EUDS and A0A0E0DTM1), Oryza glumipatula (A0A0E0B5V1), Oryza punctata (A0A0E0M3X5 and A0A0E0L4F1), Oryza barthii (A0A0D3H9G2 and A0A0D3FB13), Oryza meyeriana (A0A6G1EIM6 and A0A6G1EKK2), Panicum miliaceum (A0A3L6T9R3 and A0A3L6QP81), Panicum hallii (A0A2T7EV82), Dichanthelium oligosanthes (A0A1E5VCB3), Leersia perrieri (A0A0D9XG37 and A0A0D9WID1), Hordeum vulgare (F2ECT4, A0A287S286 and F2EIG8), Triticum aestivum (A0A3B6MUI3, A0A3B6LR90 and A0A3B6KK13), Triticum turgidum (A0A446TAY7, A0A446UHW9 and A0A446TAU4), Aegilops tauschii (A0A453LGW1, A0A453LGT1 and A0A453LH18), Elaeis guineensis (A0A6I9S0N7, A0A6J9SDQ6 and A0A6J9RAZ1), Aegilops tauschi (R7WAV9), Ananas comosus (A0A6V7NRT6, A0A6V7QAW2 and A0A6V7NKL2), Eragrostis curvula (A0A5J9TG58), Musa balbisiana (A0A4V4H9E0 and A0A4S8JRH4), Triticum urartu (M8AAQ1), Zostera marina (A0A0K9PM16).

### Components of Progression Through the First Division of Meiosis

In some embodiments, the complete, partial, or partially-complemented MiMe genotype comprises one or more MiMe alleles conferring decreased expression of one or more MiMe loci of a component of progression through the first division of meiosis. Components of progression through the first division of meiosis include, for example, both (a) MiMe loci that encode gene products that are required for progression through the first division of meiosis and (b) MiMe loci that that are associated with first division restitution mechanisms, since the effective end result of both is as if the first division of meiosis did not occur, with the resulting gametes of each containing non-sister chromatids. First division restitution (also known as nuclear restitution) mechanisms are known in the art and described in Peloquin et al. (1999. Meiotic mutants in potato: valuable variants. Genetics 153: 1493-1499) and Brownfield and Kohler (2010. Unreduced gamete formation in plants: mechanisms and prospects. J Exp Bot 62:5, 1659-1668). In certain embodiments, the one or more MiMe loci of the component of progression through the first division of meiosis comprise PS1, JASON, or a combination thereof. One of skill in the art will understand that MiMe loci of the component of progression through the first division of meiosis are not limited to PS1 and JASON, and may include any loci encoding gene products required for progression through the first division of meiosis or associated with first division restitution mechanisms. By way of example only, a gene product of the component of progression through the first division of meiosis is exemplified by PS1 protein or a PS1-like protein, and specifically by the PS1 and PS1-like protein sequences and percent identities described herein. Representative PS1 protein sequences (SEQ ID NOs: 80 and 85) and a representative PS1-like protein sequence (SEQ ID NO: 86) are provided in the sequence listing as outlined in Table 9. The gene products of MiMe loci of the component of progression through the first division of meiosis include: a) PS1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the PS1 protein of SEQ ID NO: 80 or 85; and b) PS1-like proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the PS1-like protein of SEQ ID NO: 86.

There is an abundance of known PS1 gene products from both dicotyledonous plants (dicots) and monocotyledonous plants (monocots) including all of the following, which sequences are hereby incorporated by reference in their form as of the effective filing date. Exemplary PS1 gene products from dicots include those of, by way of example, Rubus occidentalis (MAKER: Ro02_G24292, Ro03_G10148 and Ro06_G22205), Vaccinium corymbosum (MAKER: VaccDscaff6-augustus-gene-193.35-mRNA-1, VaccDscaff38-augustus-gene-176.29-mRNA-1 and VaccDscaff37-augustus-gene-104.17-mRNA-1), Arabidopsis thaliana (NP_001320841.1, NP_564445.1 and NP_001320842.1), Arabidopsis lyrata (XP 020868640.1, D7KK60 and D7MJU2), Camelina sativa (XP 019099370.1, XP 010478868.1 and XP 010461265.1), Eutrema salsugineum (XP 024009120.1 and V4KTJ3), Brassica oleracea (XP 013606236.1, XP 013588483.1 and XP 013588484.1), Brassica napus (XP 022566719.1, XP 013666939.2 and XP 013715052.1), Raphanus sativus (XP 018457892.1, XP 018449411.1 and XP 018491194.1), Brassica rapa (XP 009121278.3, XP 009124409.3 and XP 009107929.1), Tarenaya hassleriana (XP 010538917.1), Citrus sinensis (XP 015384257.1, XP 006473209.1 and A0A067H883), Quercus lobata (XP 030952045.1, XP 030952047.1 and XP 030952046.1), Quercus suber (XP 023905595.1, XP 023905593.1 and XP 023905594.1), Camellia sinensis (XP 028095050.1), Manihot esculenta (XP 021600768.1, XP 021600769.1 and A0A2C9U300), Vitis riparia (XP 034676287.1 and XP 034675847.1), Vitis vinifera (XP 002267136.3, A0A438EMM8 and D7SRA9), Carica papaya (XP 021895984.1), Prunus avium (XP 021831870.1 and A0A6P5TXV0), Herrania umbratica (XP 021300133.1 and A0A6J1BM09), Populus alba (XP 034896994.1 and A0A4U5QYS1), Populus trichocarpa (XP 006376313.2 and A0A2K1Y4D3), Citrus clementina (XP 024040904.1 and V4SJG2), Syzygium oleosum (XP 030468012.1), Fragaria vesca (XP 011458433.1), Cannabis sativa (XP 030509036.1, XP 030479265.1 and XP 030510860.1), Jatropha curcas (XP 012081093.1, XP 012081094.1 and XP 012081092.1), Solanum tuberosum (XP 006351164.1, XP 006353632.1 and M1AJ10), Theobroma cacao (XP 007020277.2 and A0A061FLD5), Hevea brasiliensis (XP 021647538.1, XP 021647539.1 and A0A6A6L7M1), Prunus persica (XP 020425140.1, A0A251MTL2 and M5VNZ4), Eucalyptus grandis (XP 018731880.1, XP 018731879.1 and XP 010064797.1), Rhodamnia argentea (XP 030525935.1, XP 030525929.1 and XP 030525921.1), Solanum pennellii (XP 015057240.1 and XP 015080025.1), Populus euphratica (XP 011038157.1), Solanum lycopersicum (XP 010312823.1, XP 004241820.1 and A0A3Q7IT06), Prunus mume (XP 008237297.1), Lupinus angustifolius (XP 019449797.1, XP 019413475.1 and XP 019413474.1), Rosa chinensis (XP 024163279.1, XP 024163280.1 and XP 024163278.1), Prunus dulcis (XP 034196761.1, A0A5E4GBW0 and A0A5E4GC86), Nicotiana attenuata (XP 019224985.1, XP 019239966.1 and A0A314KUF9), Nicotiana tabacum (XP 016457121.1, XP 016487625.1 and A0A1S3YXQ4), Nicotiana tomentosiformis (XP 033516218.1 and XP 018631965.1), Juglans regia (XP 018814993.1 and A0A2I4E6H5), Nicotiana sylvestris (XP 009801048.1, A0A1U7YD44 and A0A1U7VW00), Ricinus communis (XP 002526845.2 and B9SLH6), Durio zibethinus (XP 022772396.1 and A0A6P6B672), Coffea arabica (XP 027093289.1, XP 027090854.1 and A0A6P6URV7), Coffea eugenioides (XP 027168560.1), Sesamum indicum (XP 011097120.1 and A0A6I9UFJ1), Cucumis sativus (XP 004139758.3 and A0A0A0K684), Pyrus x (XP 009375297.1), Ziziphus jujuba (XP 015886336.1 and A0A6P4A0P1), Phaseolus vulgaris (XP 007151890.1, XP 007143660.1 and V7C174), Spinacia oleracea (XP 021849410.1 and A0A0K9R6X0), Chenopodium quinoa (XP 021760453.1, XP 021748664.1 and XP 021719428.1), Abrus precatorius (XP 027348090.1, XP 027367743.1 and XP 027367744.1), Punica granatum (XP 031393134.1, XP 031393133.1 and A0A218W5H8), Vigna unguiculata (XP 027923336.1, XP 027935177.1 and A0A4D6M0N7), Arachis duranensis (XP 020988894.1, A0A6P5MVW9 and A0A6P4BSX4), Arachis hypogaea (XP 025623533.1, A0A445B534 and A0A444X5H5), Malus domestica (XP 008382009.2, XP 008381002.1 and A0A498JY65), Momordica charantia (XP 022135216.1, XP 022135215.1 and A0A6J1C011), Gossypium arboreum (XP 017611000.1, XP 017610999.1 and A0A6P4MGP0), Gossypium raimondii (XP 012485444.1, XP 012485443.1 and A0A0D2RVJ0), Capsicum annuum (XP 016569746.1, XP 016574880.1 and A0A2G2ZP37), Vigna radiata (XP 014497684.1, XP 014497769.1 and XP 014512549.1), Cucurbita moschata (XP 022932036.1, XP 022932037.1 and A0A6J1EVI9), Morus notabilis (XP 024020360.1 and W9QXH1), Cucurbita pepo (XP 023523475.1 and XP 023523476.1), Gossypium hirsutum (XP 016671642.1, XP 016669250.1 and A0A1U8HT95), Glycine max (XP 003519963.1, XP 003548950.1 and XP 006589449.1), Cucurbita maxima (XP 022972928.1, XP 022972927.1 and A0A6J1I652), Cucumis melo (XP 008461472.1 and A0A1S3CF74), Olea europaea (XP 022858067.1 and XP 022866668.1), Glycine soja (XP 028199311.1, XP 028182421.1 and XP 028182419.1), Erythranthe guttata (XP 012854718.1 and A0A022RWW3), Beta vulgaris (XP 010675736.1 and XP 010675813.1), Vigna angularis (XP 017438284.1, XP 017412791.1 and XP 017412790.1), Ipomoea triloba (XP 031097565.1), Ipomoea nil (XP 019180660.1, XP 019180661.1 and XP 019180662.1), Daucus carota (XP 017244092.1 and A0A162A937), Cajanus cajan (XP 020225466.1, XP 020225463.1 and A0A151QT36), Pistacia vera (XP 031287694.1, XP 031287697.1 and XP 031287696.1), Medicago truncatula (XP 003591991.2, G7I9P5 and A0A396JVH4), Cicer arietinum (XP 004496355.1 and A0A1S2XY53), Cynara cardunculus (XP 024969758.1 and A0A124SGJ2), Lactuca sativa (XP 023742219.1 and A0A2J6JX58), Helianthus annuus (XP 035833749.1, XP 021983704.1 and XP 021983705.1), Arabis alpina (A0A087HSN6, A0A087GTM0 and A0A087GTM1), Arabis nemorensis (A0A565CDI7), Brassica oleracea (A0A3P6DL93, A0A3P6GM62 and A0A3P6G4S4), Brassica campestris (A0A397XN88, A0A3P6DAQ0 and A0A397XWI5), Microthlaspi erraticum (A0A6D2HZV2 and A0A6D2KQX4), Brassica rapa (M4CFC6, M4FIP0 and M4F6N9), Citrus unshiu (A0A2H5NIA3), Camellia sinensis (A0A4S4DFU4), Acer yangbiense (A0A5C7H7X4), Actinidia chinensis (A0A2R6RP98), Fagus sylvatica (A0A2N9I7E3), Nyssa sinensis (A0A5J5BYJ0), Brassica cretica (A0A3N6SHY0), Hibiscus syriacus (A0A6A2YGJ6 and A0A6A3CHR5), Cephalotus follicularis (A0A1Q3CI52), Carpinus fangiana (A0A5N6RLK7, A0A5N6RL88 and A0A5N6RMS0), Solanum chilense (A0A6N2ANG5 and A0A6N2CGN4), Lupinus albus (A0A6A5PGY6, A0A6A5PQA7 and A0A6A4R5W1), Prunus armeniaca (A0A6J5Y150 and A0A6J5VN91), Corchorus capsularis (A0A1R3JV35), Trema orientale (A0A2P5FQZ3), Coffea canephora (A0A068VFF4), Salix brachista (A0A5N5KI08 and A0A5N5LYQ1), Parasponia andersonii (A0A2P5E5J9), Morella rubra (A0A6A1W5X9 and A0A6A1V0J5), Corchorus olitorius (A0A1R3HC73), Gossypium tomentosum (A0A5D2P2V6, A0A5D2P2A9 and A0A5D2JHN0), Capsicum baccatum (A0A2G2WY45 and A0A2G2WKG1), Capsicum chinense (A0A2G3CMM6 and A0A2G3C841), Gossypium mustelinum (A0A5D2XWS3, A0A5D2XXA5 and A0A5D2TIU4), Gossypium darwinii (A0A5D2B938, A0A5D2BBF4 and A0A5D2F8W6), Malus baccata (A0A540M8A9 and A0A540NG96), Solanum chacoense (F5A7N5, F5A7N4 and F5A7P1), Gossypium barbadense (A0A5J5Q7Q2, A0A5J5Q2B7 and A0A5J5UER6), Pyrus ussuriensis (A0A5N5I8H1 and A0A5N5FQ55), Vigna angularis (A0A0S3T9K7 and A0A0S3SNM3), Cucumis melo (A0A5A7V6C9), Salvia splendens (A0A4D8ZRL6, A0A4D8XWB1 and A0A4D9BXU1), Phaseolus angularis (A0A0L9VLY7, A0A0L9U029 and A0A0L9VL32), Gossypium australe (A0A5B6UEZ4), Dorcoceras hygrometricum (A0A2Z7BG28), Salix viminalis (A0A6N2MEG9, A0A6N2LZF9 and A0A6N2MA94), Striga asiatica (A0A5A7P3Z7 and A0A5A7PYQ6), Trifolium pratense (A0A2K3P168), Cuscuta campestris (A0A484MEH0 and A0A484KSB0), Prunus yedoensis (A0A314UU47), Cuscuta australis (A0A328D2Y5), Mikania micrantha (A0A5N6NI92), Solanum chacoense (A0A0V0H9T7 and A0A0V0GWE7), Lactuca saligna (A0A6S7MZ77), Trifolium medium (A0A392PDS8), Citrullus lanatus (MAKER: Cla97C09G173120.1 and ClCG09G010400.1). Exemplary PS1 gene products from monocots include those of, by way of example, Brachypodium distachyon (XP 010239277.1 and I1IN51), Oryza sativa (XP 015615537.1), Oryza brachyantha (XP 015697772.1, XP 015694339.1 and J3N6D2), Zea mays (NP_001348386.1, XP 035820777.1 and XP 008668271.1), Aegilops tauschii (XP 020158719.1 and XP 020158718.1), Setaria viridis (XP 034606561.1, XP 034606560.1 and A0A4U6TBY4), Setaria italica (XP 012703818.1, XP 004978825.2 and K3ZH00), Panicum hallii (XP 025827253.1, XP 025827254.1 and A0A2T8I7Q6), Sorghum bicolor (XP 021316557.1 and A0A1Z5RHG0), Phoenix dactylifera (XP 017697310.1, XP 017697192.1 and A0A2H3ZLM8), Elaeis guineensis (XP 010912566.2 and XP 010911914.2), Ananas comosus (XP 020106558.1, A0A6P5GKG8 and A0A199VSP2), Musa acuminata (XP 018675137.1, XP 018675135.1 and XP 018683627.1), Asparagus officinalis (XP 020253166.1 and A0A5P1FKL5), Phalaenopsis equestris (XP 020583806.1), Dendrobium catenatum (XP 020693391.1 and A0A2I0W4F5), Oryza sativa (Q0IUA5, Q53P84 and B9G9M0), Oryza nivara (A0A0E0IYS9), Oryza sativa (B8BJB8), Oryza glumipatula (A0A0E0BFV0), Oryza rufipogon (A0A0E0R4S4), Oryza glaberrima (I1QY34), Oryza barthi (A0A0D3HIR4), Oryza meridionalis (A0A0E0F2Z2), Oryza punctata (A0A0E0MCY0), Oryza meyeriana (A0A6G1C756), Hordeum vulgare (A0A287NG49, A0A287NGL6 and A0A287NGM1), Triticum turgidum (A0A446R3M3, A0A446R396 and A0A446R3B2), Triticum aestivum (A0A3B6HW94, A0A3B6HUN9 and A0A3B6HYM4), Leersia perrieri (A0A0D9XPJ1, A0A0D9XPJ0 and A0A0D9XPI8), Panicum miliaceum (A0A3L6PMA2 and A0A3L6Q1S3), Panicum hallii (A0A2T7CKM4), Aegilops tauschi (A0A453HLP9, A0A453HLP6 and A0A453HLP3), Triticum urartu (M7YX52), Eragrostis curvula (A0A5J9US36 and A0A5J9SPK5), Aegilops tauschi (M8AS18), Elaeis guineensis (A0A6I9QSD8 and A0A6I9QMW2), Ananas comosus (A0A6V7QXN0, A0A6V7PBP8 and A0A6V7PYK9), Zostera marina (A0A0K9P3Q9), Musa balbisiana (A0A4S8IY84 and A0A4S8IP24).

By way of example only, a gene product of the component of progression through the first division of meiosis is also exemplified by a JASON protein (e.g., a JASON-1 or a JASON-2 protein), and specifically by the JASON protein sequences and percent identities described herein. Representative JASON protein sequences (SEQ ID NOs: 76, 77, and 87), including a representative JASON-1 protein sequence (SEQ ID NO: 76) and a representative JASON-2 protein sequence (SEQ ID NO: 77), are provided in the sequence listing as outlined in Table 9. The gene products of MiMe loci of the component of progression through the first division of meiosis include: a) JASON-1 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the JASON-1 protein of SEQ ID NO: 76; b) JASON-2 proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the JASON-2 protein of SEQ ID NO: 77; and b) JASON proteins having at least 40%, 45%, 50%, 55%, 60%, 65%, 70%, 75%, 80%, 85%, 90%, 95% or 98% sequence identity, or at least 60%, 65%, 70%, 75%, 80%, 85%, 90%, 95% or 98% sequence similarity to the JASON protein of SEQ ID NO: 87.

There is an abundance of known JASON gene products from both dicotyledonous plants (dicots) and monocotyledonous plants (monocots) including all of the following, which sequences are hereby incorporated by reference in their form as of the effective filing date. Exemplary JASON gene products from dicots include those of, by way of example, Rubus occidentalis (MAKER: Ro06_G13343), Vaccinium corymbosum (MAKER: VaccDscaff27-augustus-gene-180.23-mRNA-1, VaccDscaff1402-augustus-gene-0.6-mRNA-1 and VaccDscaff45-snap-gene-175.22-mRNA-1), Arabidopsis lyrata (XP 002892352.1, XP 020884374.1 and D7KG54), Arabidopsis thaliana (NP_172151.2, F4IDQ5 and A0A5S9SYD9), Camelina sativa (XP 010475403.1, XP 010457790.1 and XP 010462500.1), Raphanus sativus (XP 018451141.1, XP 018472588.1 and XP 018474146.1), Eutrema salsugineum (XP 006417897.1, V4KES0 and V4M5V9), Brassica oleracea (XP 013586399.1, XP 013602509.1 and XP 013634220.1), Brassica napus (XP 013694276.2, XP 022546198.1 and XP 013683238.1), Brassica rapa (XP 009110978.1, XP 018511221.1 and XP 033144940.1), Tarenaya hassleriana (XP 010557153.1, XP 010522056.1 and XP 010522057.1), Carica papaya (XP 021898299.1, XP 021898300.1 and XP 021888540.1), Populus euphratica (XP 011026219.1, XP 011026221.1 and XP 011032779.1), Populus alba (XP 034915075.1, XP 034931169.1 and XP 034931170.1), Populus trichocarpa (XP 024451939.1, XP 024456185.1 and A0A3N7EP17), Herrania umbratica (XP 021273988.1 and A0A6J0ZGB7), Theobroma cacao (XP 007048945.2, XP 007048946.2 and A0A061DKZ1), Jatropha curcas (XP 012073983.1 and A0A067KXD5), Durio zibethinus (XP 022759898.1, XP 022759900.1 and A0A6P6A4H0), Hevea brasiliensis (XP 021678466.1, XP 021687582.1 and XP 021659874.1), Prunus avium (XP 021824844.1, XP 021824845.1 and XP 021831981.1), Quercus lobata (XP 030931979.1, XP 030931980.1 and XP 030931981.1), Ricinus communis (XP 015581488.1, XP 002530206.2 and B9SW37), Prunus mume (XP 008229580.1 and XP 008235614.1), Quercus suber (XP 023894468.1, XP 023894469.1 and XP 023894470.1), Pistacia vera (XP 031257377.1 and XP 031257382.1), Prunus dulcis (XP 034210497.1, XP 034210498.1 and XP 034196964.1), Prunus persica (XP 020414309.1, XP 020414310.1 and XP 007199872.1), Citrus clementina (XP 024042487.1, XP 006437114.1 and V4VD94), Citrus sinensis (XP 024956253.1, XP 006484983.1 and A0A067DH64), Cannabis sativa (XP 030484860.1, XP 030484861.1 and XP 030484862.1), Gossypium hirsutum (XP 016746989.1, XP 016746990.1 and XP 016746024.1), Gossypium arboreum (XP 017625891.1, A0A0B0NZI4 and A0A6P4ND02), Gossypium raimondii (XP 012488768.1, XP 012488776.1 and A0A0D2Q289), Pyrus x (XP 009377072.1, XP 009356595.1 and XP 009356597.1), Momordica charantia (XP 022136916.1 and A0A6J1C4V0), Morus notabilis (XP 010093175.1 and W9R0Q8), Manihot esculenta (XP 021621959.1, XP 021603466.1 and XP 021603465.1), Juglans regia (XP 018835614.1, XP 035540709.1 and A0A2I4FVD9), Malus domestica (XP 008380178.1, XP 008342335.2 and XP 008380179.1), Fragaria vesca (XP 011467606.1), Cucurbita maxima (XP 022980689.1, XP 022985306.1 and XP 022985307.1), Rosa chinensis (XP 024180318.1, XP 024180316.1 and XP 024180314.1), Cucurbita moschata (XP 022942512.1, XP 022931533.1 and XP 022931534.1), Cucurbita pepo (XP 023539166.1, XP 023539175.1 and XP 023553554.1), Ziziphus jujuba (XP 024928697.1 and A0A6P6G3G4), Vitis riparia (XP 034701376.1 and XP 034701367.1), Vitis vinifera (XP 002283205.1, A0A438HCW7 and F6H581), Camellia sinensis (XP 028105866.1, XP 028105867.1 and XP 028105868.1), Syzygium oleosum (XP 030466722.1 and XP 030462822.1), Rhodamnia argentea (XP 030538612.1, XP 030519372.1 and XP 030519377.1), Cucumis melo (XP 008451829.1 and A0A1S3BRU1), Eucalyptus grandis (XP 010053987.1, XP 010035945.1 and XP 010035944.1), Cucumis sativus (XP 004147749.1, XP 031739890.1 and A0A0A0L089), Coffea arabica (XP 027063407.1, XP 027063408.1 and XP 027101796.1), Coffea eugenioides (XP 027171250.1 and XP 027171251.1), Lupinus angustifolius (XP 019435062.1, XP 019435056.1 and XP 019435069.1), Punica granatum (XP 031390018.1, A0A6P8DFD4 and A0A2I0L9C7), Arachis hypogaea (XP 025668210.1, XP 025668211.1 and XP 025608725.1), Daucus carota (XP 017244605.1, XP 017257130.1 and XP 017238463.1), Medicago truncatula (XP 024633597.1, XP 013459649.2 and XP 024639377.1), Cicer arietinum (XP 027190958.1, XP 004499969.1 and XP 004495071.1), Ipomoea triloba (XP 031128547.1, XP 031099008.1 and XP 031099009.1), Sesamum indicum (XP 020551402.1, XP 011100505.1 and XP 011100506.1), Ipomoea nil (XP 019162337.1 and XP 019182282.1), Arachis ipaensis (XP 020962713.1, XP 016167422.1 and XP 020962712.1), Olea europaea (XP 022871861.1 and XP 022865334.1), Vigna unguiculata (XP 027931047.1, XP 027939700.1 and A0A4D6KSD9), Vigna angularis (XP 017435235.1, XP 017429588.1 and XP 017415446.1), Cynara cardunculus (XP 024995337.1, XP 024989919.1 and XP 024981813.1), Glycine soja (XP 028212613.1, XP 028212612.1 and XP 028220024.1), Cajanus cajan (XP 020204322.1, XP 020206653.1 and A0A151R663), Nicotiana tabacum (XP 016457396.1, XP 016455145.1 and XP 016477562.1), Solanum lycopersicum (XP 004243611.1, XP 004251552.1 and A0A3Q7HD41), Nicotiana sylvestris (XP 009771902.1, XP 009795643.1 and XP 009785070.1), Nicotiana tomentosiformis (XP 009603107.1, XP 009628893.1 and XP 009589704.1), Nicotiana attenuata (XP 019264475.1, XP 019257514.1 and XP 019232794.1), Solanum pennellii (XP 015080467.1 and XP 015059725.1), Vigna radiata (XP 014503964.1, XP 014513791.1 and XP 014513790.1), Glycine max (XP 006575641.1, NP_001239769.1 and I1N107), Erythranthe guttata (XP 012830139.1, XP 012830140.1 and XP 012830769.1), Abrus precatorius (XP 027368570.1), Lactuca sativa (XP 023736559.1, XP 023736895.1 and A0A2J6K9L2), Chenopodium quinoa (XP 021735274.1, XP 021724579.1 and XP 021763290.1), Solanum tuberosum (XP 006352001.1, XP 015160469.1 and M1CT79), Capsicum annuum (XP 016541235.1, XP 016580363.1 and A0A1U8HGR3), Arachis duranensis (XP 015943693.1, XP 020983750.1 and XP 015943694.1), Beta vulgaris (XP 010688901.1), Phaseolus vulgaris (XP 007146236.1, XP 007144862.1 and V7BNS5), Spinacia oleracea (XP 021865022.1, XP 021847325.1 and A0A0K9QQ90), Helianthus annuus (XP 021997562.1, XP 021997561.1 and XP 021997560.1), Arabis nemorensis (A0A565ALB2), Microthlaspi erraticum (A0A6D2IDS1, A0A6D2IZL6 and A0A6D2J1R7), Brassica oleracea (A0A3P6EP37, A0A3P6F7W8 and A0A3P6AML3), Brassica campestris (A0A3P6CU05, A0A397YGD0 and A0A3P6CEU9), Arabis alpina (A0A087HLI1), Brassica rapa (M4EPD9, M4DG93 and M4DYS1), Brassica cretica (A0A3N6PX65 and A0A3N6QRA2), Corchorus olitorius (A0A1R3KSQ8), Corchorus capsularis (A0A1R3K916), Salix brachista (A0A5N5NMJ7 and A0A5N5MQM8), Trema orientale (A0A2P5EVX6), Gossypium mustelinum (A0A5D2YNY1, A0A5D2UC18 and A0A5D2UAM7), Gossypium barbadense (A0A5J5V5D0, A0A5J5R0D7 and A0A5J5V5M6), Morella rubra (A0A6A1VBU9), Gossypium darwinii (A0A5D2FY15, A0A5D2C242 and A0A5D2G033), Acer yangbiense (A0A5C7HCF6), Parasponia andersonii (A0A2P5BTL5), Gossypium tomentosum (A0A5D2KA41, A0A5D2PV54 and A0A5D2KCL5), Populus davidiana (A0A6M2EUI2), Citrus unshiu (A0A2H5QGX7), Malus baccata (A0A540L9D3 and A0A540L2H0), Salix viminalis (A0A6N2NM07), Carpinus fangiana (A0A5N6KZB0), Prunus armeniaca (A0A6J5WXM4 and A0A6J5UDK3), Hibiscus syriacus (A0A6A2ZF14, A0A6A3CM00 and A0A6A2Z0N9), Gossypium australe (A0A5B6UP38), Nyssa sinensis (A0A5J5A2F4 and A0A5J5A6W5), Prunus yedoensis (A0A314YZE6), Pyrus ussuriensis (A0A5N5GJ38 and A0A5N5G628), Fagus sylvatica (A0A2N9F153, A0A2N9FU50 and A0A2N9F163), Cephalotus follicularis (A0A1Q3DDT2), Lupinus albus (A0A6A5M6K2, A0A6A4QMZ9 and A0A6A4P8Y9), Coffea canephora (A0A068UIS3 and A0A068UJH1), Cucumis melo (A0A5D3D147 and A0A5A7V7I7), Actinidia chinensis (A0A2R6P308 and A0A2R6PTT6), Camellia sinensis (A0A4S4E772), Trifolium pratense (A0A2K3PB82 and A0A2K3MY71), Trifolium subterraneum (A0A2Z6NAB1), Phaseolus angularis (A0A0L9VAA7 and A0A0L9UUP3), Vigna angularis (A0A0S3S7Q1 and A0A0S3RJV6), Solanum chacoense (A0A0V0IDT0), Solanum chilense (A0A6N2AU42), Cuscuta australis (A0A328D5Z5 and A0A328DTP9), Cuscuta campestris (A0A484MQX0, A0A484L2D1 and A0A484L104), Striga asiatica (A0A5A7QTF4, A0A5A7R9A4 and A0A5A7PLH1), Dorcoceras hygrometricum (A0A2Z7BGP7), Lactuca saligna (A0A6S7MHR2 and A0A6S7P2U5), Mikania micrantha (A0A5N6MPK9 and A0A5N6LEZ0), Salvia splendens (A0A4D9AUH9, A0A4D8ZDY9 and A0A4D9AR03), Capsicum baccatum (A0A2G2X2X4 and A0A2G2WC48), Capsicum chinense (A0A2G3BN47 and A0A2G3BZS1), Trifolium medium (A0A392Q4Q9), Citrullus lanatus (MAKER: Cla97C06G110790.1, Cla97C07G137490.1 and Cla97C02G026610.1). Exemplary JASON gene products from monocots include those of, by way of example, Oryza sativa (XP 015628653.1, XP 015628655.1 and XP 015628654.1), Brachypodium distachyon (XP 003559372.1, XP 010240601.1 and XP 010240600.1), Oryza brachyantha (XP 006650673.1, XP 015689050.1 and XP 015693677.1), Aegilops tauschii (XP 020195562.1, XP 020178895.1 and XP 020161298.1), Sorghum bicolor (XP 021313820.1, XP 021314324.1 and XP 021306240.1), Setaria viridis (XP 034574917.1, XP 034590082.1 and XP 034590084.1), Panicum hallii (XP 025795719.1, XP 025811230.1 and XP 025811231.1), Elaeis guineensis (XP 019705593.1, XP 010941823.1 and XP 029121598.1), Phoenix dactylifera (XP 008806685.1, XP 008777000.1 and XP 017701979.1), Musa acuminata (XP 018683059.1, XP 009406745.1 and XP 009406746.1), Ananas comosus (XP 020095644.1, XP 020095643.1 and XP 020095642.1), Asparagus officinalis (XP 020269189.1, XP 020256241.1 and XP 020247430.1), Dendrobium catenatum (XP 028550873.1, XP 020699530.1 and A0A2I0WSQ7), Phalaenopsis equestris (XP 020587721.1 and XP 020587720.1), Setaria italica (XP 004951783.1, XP 004967263.1 and XP 022681714.1), Zea mays (NP_001132267.1, XP 008647301.1 and NP_001130670.1), Oryza sativa (Q8S716, Q7G7G7 and B7FA65), Oryza nivara (A0A0E0IT87, A0A0E0IT86 and A0A0E0G1W4), Oryza barthii (A0A0D3FPQ5, A0A0D3FPQ4 and A0A0D3F173), Oryza rufipogon (A0A0E0P204, A0A0E0P203 and A0A0E0NAD9), Oryza glumipatula (A0A0D9ZDV7 and A0A0D9YMU6), Oryza sativa (B8AKL3 and B8AIH7), Oryza meridionalis (A0A0E0D7L9, A0A0E0CG88 and A0A0E0CG89), Oryza punctata (A0A0E0KJN4 and A0A0E0JW17), Oryza meyeriana (A0A6G1DS95), Leersia perrieri (A0A0D9VZP3, A0A0D9VZP4 and A0A0D9VCM6), Oryza glaberrima (I1PG10 and I1NXM2), Aegilops tauschii (M8BJQ1, M8C383 and M8BJE8), Triticum aestivum (A0A3B6MXC7, A0A3B6LU96 and A0A3B6KQ60), Aegilops tauschii (A0A453LWP9, A0A453LXT6 and A0A453LXW5), Triticum turgidum (A0A446UNZ7, A0A446UP20 and A0A446TGN6), Hordeum vulgare (A0A287SHA1, A0A287SHA9 and A0A287SH58), Triticum urartu (M7ZDK2), Panicum hallii (A0A2T7C0C4 and A0A2T7F2G1), Eragrostis curvula (A0A5J9W7Q9), Dichanthelium oligosanthes (A0A1E5ULQ9), Panicum miliaceum (A0A3L6S5L1, A0A3L6QGD2 and A0A3L6PMH6), Elaeis guineensis (A0A6J0PGY5, A0A6I9SG61 and A0A6I9SER3), Ananas comosus (A0A6V7NMQ6 and A0A6V7PUP7), Musa balbisiana (A0A4S8ISD9 and A0A4S8JJT6), Zostera marina (A0A0K9PZP8).

The sequences of the gene products listed above may be accessed using the given accession numbers in the RefGen, UniProt, and RefSeq databases, except for those marked with “MAKER”. The identifiers of the sequences of the gene products marked with “MAKER” are MAKER-derived annotations of genome sequences of Citrullus lanatus cv 97103 v2.0 (Guo S et al. 2013. The draft genome of watermelon (Citrullus lanatus) and resequencing of 20 diverse accessions. Nature Genetics 45:51-58); Citrullus lanatus cv Charleston Gray v1.0 (Wu S. et al. 2019. Genome of ‘Charleston Gray’, the principal American watermelon cultivar, and genetic characterization of 1,365 accessions in the U.S. National Plant Germplasm System watermelon collection. Plant Biotechnol J), Rubus occidentalis v3.0 (VanBuren et al. 2016. The genome of black raspberry (Rubus occidentalis). The Plant Journal. 87(6):535-547.), and Vaccinium corymbosum cv Draper v1.0 (Marivi Colle et al. 2019. Haplotype-phased genome and evolution of phytonutrient pathways of tetraploid blueberry, GigaScience, Volume 8, Issue 3).

Plants that Produce Inviable Gametes and/or Seedless Fruit

In some embodiments, germination of a seed of the population of polyploid seed or of the subpopulation of genetically uniform polyploid seed results in a sterile plant that produces inviable gametes. For example, in some embodiments, the population of polyploid seed or the subpopulation of genetically uniform polyploid seed that has a partial MiMe genotype has a wild-type meiosis phenotype. In some variations, the population of polyploid seed or the subpopulation of genetically uniform polyploid seed that has a partial MiMe genotype and has a wild-type meiosis phenotype and is triploid, pentaploid, or heptaploid, such that germination of the seed results in a sterile plant that produces inviable gametes due to the non-even ploidy of the plant. In some embodiments, germination of a seed of the population of polyploid seed or the subpopulation of genetically uniform polyploid seed results in a plant that has a partially-complemented MiMe genotype and has neither a wild-type meiosis phenotype nor a MiMe phenotype. In some embodiments, germination of a seed of the population of polyploid seed or the subpopulation of genetically uniform polyploid seed results in a plant that produces inviable gametes due to the partially complemented MiMe genotype resulting in neither wild-type meiosis or MiMe. Accordingly, in some embodiments, germination of a seed of the population of polyploid seed or the subpopulation of genetically uniform polyploid seed results in a sterile plant that produces inviable gametes. In some embodiments, germination of a seed of the population of polyploid seed or the subpopulation of genetically uniform polyploid seed results in a plant that produces seedless fruit. In some embodiments, germination of a seed of the population of polyploid seed or the subpopulation of genetically uniform polyploid seed results in a seedless plant.

### Parthenocarpy

In some embodiments, the population of polyploid seed is of a parthenocarpic plant species. In some flowering plants, particularly those with mutations affecting the synthesis, regulation, or perception of growth hormones and/or transcriptional regulatory machinery, fruit development may proceed independently of pollination, fertilization, and/or seed development in a process commonly known as parthenocarpy. This process may be further classified into three exemplary classes: stenospermocarpy, stimulative parthenocarpy, and vegetative parthenocarpy. Each of these classes differs in the stages required for fruit development. Stenospermocarpic fruits may require pollination, fertilization, and initialization of seed development for fruit formation. However, soon after initialization the embryo may be aborted and an incompletely developed seed may remain. This is the class of parthenocarpy common to, by way of example only, many seedless table grapes and watermelon varieties. Stimulative parthenocarpic fruits do not require fertilization, or seed development, but they may require pollination or at minimum an exogenous influence to initiate fruit development. One exemplary form of stimulative parthenocarpy is also known as induced or artificial parthenocarpy. In this form, the induction of fruit development without pollination, fertilization, or seed development results from exogenous application of a plant hormone such as, by way of example only, auxin, gibberellins, or cytokinin on flowers at the correct developmental stage. Another class of parthenocarpic fruits are those capable of vegetative parthenocarpy. These plants do not require pollination, fertilization, seed development, or exogenous stimulus for fruit development. Many seedless crops such as, by way of example only, bananas, cucumbers, pears, and figs commonly result from this form of parthenocarpy. Genes are known in the art which, when rendered non-functional, confer vegetative parthenocarpy, for example, in watermelon (U.S. Pat. App. Pub. No. 2019/0300900A1).

The aforementioned exemplary classes of parthenocarpy may be further defined by their ability to generate viable seeds when pollination does occur. Those plants capable of producing viable seed upon pollination and fertilization are commonly referred to as facultative parthenocarps, whereas those that never produce viable seed but still produce fruit are commonly referred to as obligate parthenocarps. In these instances of facultative parthenocarpy, seedlessness may be achieved by multiple means. For example, in the case of facultative vegetative parthenocarps, simply ensuring that no viable pollination occurs is sufficient to attain seedless fruits. This may be achieved by entirely removing viable pollen sources, such as only planting a single self-incompatible citrus variety in isolation, ensuring cytoplasmic male sterility, or, in such cases as squash and eggplant production, growing plants in cool environments or winter production greenhouses not conducive to effective pollination. In the case of facultative stenospermocarps or stimulative parthenocarps, pollination occurs but fertilization or seed development is aborted prior to completion of embryogenesis to ensure production of seedless fruit. One common commercial means to ensure the abortion of seed development and production of seedless fruit is the production of plants with odd ploidy, such as triploid bananas and watermelon. These triploids may be conceived by crossing a diploid mother with a tetraploid father plant and collecting the viable triploid seed they progenate or, in instances of exceedingly low or no viability, undergoing embryo rescue to recover aborted seeds.

### Exemplary Populations of Polyploid Seed

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, or at least about 95%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. The subpopulation of genetically uniform polyploid seed may have any complete MiMe genotype, partial MiMe genotype, or partially-complemented MiMe genotype described herein. In certain embodiments, the subpopulation of genetically uniform polyploid seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In certain embodiments, the subpopulation of genetically uniform polyploid seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid seed has a partial MiMe genotype comprising (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid seed has a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid seed has a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid seed has a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations, the one or more MiMe loci of the component of sister chromatid cohesion during the first division of meiosis comprise REC8, SWITCH1/DYAD, or a combination thereof; the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof; the one or more MiMe loci of the component of progression through the second division of meiosis comprise OSD1, CYCA1, TDM1, PC1, PC2, FC, or any combination thereof; and/or the one or more MiMe loci of the component of progression through the first division of meiosis comprise PS1, JASON, or a combination thereof. In other variations, the one or more MiMe loci of the component of sister chromatid cohesion during the first division of meiosis comprise REC8; the one or more MiMe loci of the component of DNA double strand breakage during meiotic recombination comprise PAIR1, SPO11-1, SPO11-2, or any combination thereof; the one or more MiMe loci of the component of progression through the second division of meiosis comprise OSD1, CYCA1, TDM1, or any combination thereof; and/or the one or more MiMe loci of the component of progression through the first division of meiosis comprise PS1. In certain embodiments, the population of polyploid seed is from a potato plant, a maize plant, an Arabidopsis plant, a tomato plant, a banana plant, a soybean plant, a watermelon plant, a blueberry plant, or a blackberry plant. In certain embodiments, the population of polyploid seed is from a dicotyledonous plant. In some variations, the population of polyploid seed is from a potato plant, an Arabidopsis plant, a tomato plant, a soybean plant, a watermelon plant, a blueberry plant, or a blackberry plant. In other embodiments, the population of polyploid seed is from a monocotyledonous plant. In some variations, the population of polyploid seed is from a maize plant or a banana plant. In certain embodiments, germination of a seed of the subpopulation of genetically uniform polyploid seed results in a sterile plant that produces inviable gametes, seedless fruit, or a combination thereof. In some embodiments, the subpopulation of genetically uniform polyploid seed comprises one or more polynucleotide sequences selected from the group consisting of SEQ ID NOs: 448-466, 468-471, 474-491, and 493-496. In some embodiments, the population of polyploid seed is from a maize plant, and the subpopulation of genetically uniform polyploid maize seed comprises one or more polynucleotide sequences selected from the group consisting of SEQ ID NOs: 448-451. In certain embodiments, the subpopulation of genetically uniform polyploid maize seed comprises a) a MiMe allele at one or more OSD1-2 loci, each independently comprising a polynucleotide sequence selected from the group consisting of SEQ ID NOs: 448 and 449; b) a MiMe allele at a REC8 locus comprising the polynucleotide sequence of SEQ ID NO: 450; and/or c) a MiMe allele at a SPO11-1 locus comprising the polynucleotide sequence of SEQ ID NO: 451. In some embodiments, the population of polyploid seed is from a potato plant, and the subpopulation of genetically uniform polyploid potato seed comprises one or more polynucleotide sequences selected from the group consisting of SEQ ID NOs: 464-496. In certain embodiments, the subpopulation of genetically uniform polyploid potato seed comprises a MiMe allele at one or more SPO11-1 loci, each independently comprising a polynucleotide sequence selected from the group consisting of SEQ ID NOs: 464-466. In certain embodiments, the subpopulation of genetically uniform polyploid potato seed comprises a) a MiMe allele at one or more REC8 loci, each independently comprising a polynucleotide sequence selected from the group consisting of SEQ ID NOs: 468 and 469; and/or b) a MiMe allele at one or more SPO11-1 loci, each independently comprising a polynucleotide sequence selected from the group consisting of SEQ ID NOs: 470 and 471. In certain embodiments, the subpopulation of genetically uniform polyploid potato seed comprises a) a MiMe allele at one or more REC8 loci, each independently comprising a polynucleotide sequence selected from the group consisting of SEQ ID NOs: 474 and 475; and/or b) a MiMe allele at one or more SPO11-1 loci, each independently comprising a polynucleotide sequence selected from the group consisting of SEQ ID NOs: 476 and 477. In certain embodiments, the subpopulation of genetically uniform polyploid potato seed comprises a MiMe allele at one or more SPO11-1 loci, each independently comprising a polynucleotide sequence selected from the group consisting of SEQ ID NOs: 478 and 479. In certain embodiments, the subpopulation of genetically uniform polyploid potato seed comprises a) a MiMe allele at one or more C1CAI loci, each independently comprising a polynucleotide sequence selected from the group consisting of SEQ ID NOs: 480 and 481; b) a MiMe allele at one or more REC8 loci, each independently comprising a polynucleotide sequence selected from the group consisting of SEQ ID NOs: 482 and 483; and/or c) a MiMe allele at one or more SPO11-1 loci, each independently comprising a polynucleotide sequence selected from the group consisting of SEQ 11 NOs: 484 and 485 In certain embodiments, the subpopulation of genetically uniform polyploid potato seed comprises a) a MiMe allele at one or more CCA1 loci, each independently comprising a polynucleotide sequence selected from the group consisting of SEQ ID NOs: 486 and 487; b) a MiMe allele at one or more REC8 loci, each independently comprising a polynucleotide sequence selected from the group consisting of SEQ ID NOs: 488 and 489; and/or c) a MiMe allele at one or more SPO11-1 loci, each independently comprising polynucleotide sequence selected from the group consisting of SEQ ID NOs: 490 and 491. In certain embodiments, the subpopulation of genetically uniform polyploid potato seed comprises a) a MiMe allele at one or more REC8 loci, each independently comprising a polynucleotide sequence selected from the group consisting of SEQ ID NOs: 493 and 494: and/or b) a MiMe allele at one or more SPO11-1 loci, each independently comprising a polynucleotide sequence selected from the group consisting of SEQ ID NOs: 495 and 496. In some embodiments, the population of polyploid seed is from an Arabidopsis plant, and the subpopulation of genetically uniform polyploid Arabidopsis seed comprises one or more polynucleotide sequences selected from the group consisting of SEQ ID NOs: 452-463. In certain embodiments, the subpopulation of genetically uniform polyploid Arabidopsis seed comprises a) a MiMe allele at one or more OSD1 loci, each independently comprising a polynucleotide sequence selected from the group consisting of SEQ ID NOs: 452 and 453; b) a MiMe allele at one or more REC8 loci, each independently comprising a polynucleotide sequence selected from the group consisting of SEQ ID NOs: 454 and 455; and/or c) a MiMe allele at one or more SPO11-1 loci, each independently comprising a polynucleotide sequence selected from the group consisting of SEQ ID NOs: 456 and 457. In certain embodiments, the subpopulation of genetically uniform polyploid Arabidopsis seed comprises a) a MiMe allele at one or more OSD1 loci, each independently comprising a polynucleotide sequence selected from the group consisting of SEQ ID NOs: 458 and 459; b) a MiMe allele at one or more REC8loci, each independently comprising a polynucleotide sequence selected from the group consisting of SEQ ID NOs: 460 and 461; and/or c) a MiMe allele at one or more PAIR1 loci, each independently comprising a polynucleotide sequence selected from the group consisting of SEQ ID NOs: 462 and 463. In some variations, each the one or more OSD1 loci, each of the one or more REC8 loci, each of the one or more PAIR1 loci, and/or each of the one or more SPO11-1 loci are present on a different homologous chromosome.

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, or at least about 95%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. The subpopulation of genetically uniform polyploid seed may have any complete MiMe genotype, partial MiMe genotype, or partially-complemented MiMe genotype described herein. In certain embodiments, the subpopulation of genetically uniform polyploid seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In certain embodiments, the subpopulation of genetically uniform polyploid seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid seed has a partial MiMe genotype comprising (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid seed has a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid seed has a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid seed has a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations, the one or more MiMe loci of the component of sister chromatid cohesion during the first division of meiosis comprise REC8; the one or more MiMe loci of the component of DNA double strand breakage during meiotic recombination comprise PAIR1, SPO11-1, SPO11-2, or any combination thereof; the one or more MiMe loci of the component of progression through the second division of meiosis comprise OSD1, CYCA1, or a combination thereof; and/or the one or more MiMe loci of the component of progression through the first division of meiosis comprise PS1. In certain embodiments, the population of polyploid seed is from a potato plant, a maize plant, or an Arabidopsis plant. In certain embodiments, germination of a seed of the subpopulation of genetically uniform polyploid seed results in a sterile plant that produces inviable gametes, seedless fruit, or a combination thereof. In some embodiments, the subpopulation of genetically uniform polyploid seed comprises one or more polynucleotide sequences selected from the group consisting of SEQ ID NOs: 448-466, 468-471, 474-491, and 493-496. In some embodiments, the population of polyploid seed is from a maize plant, and the subpopulation of genetically uniform polyploid maize seed comprises one or more polynucleotide sequences selected from the group consisting of SEQ ID NOs: 448-451. In certain embodiments, the subpopulation of genetically uniform polyploid maize seed comprises a) a MiMe allele at one or more OSD1-2 loci, each independently comprising a polynucleotide sequence selected from the group consisting of SEQ ID NOs: 448 and 449; b) a MiMe allele at a REC8 locus comprising the polynucleotide sequence of SEQ ID NO: 450; and/or c) a MiMe allele at a SPO11-1 locus comprising the polynucleotide sequence of SEQ ID NO: 451 In some embodiments, the population of polyploid seed is from a potato plant, and the subpopulation of genetically uniform polyploid potato seed comprises one or more polynucleotide sequences selected from the group consisting of SEQ ID NOs: 464-496. In certain embodiments, the subpopulation of genetically uniform polyploid potato seed comprises a MiMe allele at one or more SPO11-1 loci, each independently comprising a polynucleotide sequence selected from the group consisting of SEQ 11 NOs: 464-466. In certain embodiments, the subpopulation of genetically uniform polyploid potato seed comprises a) a MiMe allele at one or more REC8 loci, each independently comprising a polynucleotide sequence selected from the group consisting of SEQ ID NOs: 468 and 469; and/or c) a MiMe allele at one or more SPO11-1 loci, each independently comprising a polynucleotide sequence selected from the group consisting of SEQ ID NOs: 470 and 471. In certain embodiments, the subpopulation of genetically uniform polyploid potato seed comprises a) a MiMe allele at one or more REC8 loci, each independently comprising a polynucleotide sequence selected from the group consisting of SEQ ID NOs: 474 and 475; and/or b) a MiMe allele at one or more SPO11-1 loci, each independently comprising a polynucleotide sequence selected from the group consisting of SEQ ID NOs: 476 and 477. In certain embodiments, the subpopulation of genetically uniform polyploid potato seed comprises a MiMe allele at one or more SPO11-1 loci, each independently comprising a polynucleoside sequence selected from the group consisting of SEQ ID NOs: 478 and 479. In certain embodiments, the subpopulation of genetically uniform polyploid potato seed comprises a) a MiMe allele at one or more CYCA1 loci, each independently comprising a polynucleotide sequence selected from the group consisting of SEQ ID NOs: 480 and 481; b) a MiMe allele at one or more REC8 loci, each independently comprising a polynucleotide sequence selected from the group consisting of SEQ ID NOs: 482 and 483; and/or c) a MiMe allele at one or more SPO11-1 loci, each independently comprising a polynucleotide sequence selected from the group consisting of SEQ ID NOs: 484 and 485. In certain embodiments, the subpopulation of genetically uniform polyploid potato seed comprises a) a MiMe allele at one or more CYCA1 loci, each independently comprising a polynucleotide sequence selected from the group consisting of SEQ ID NOs: 486 and 487; b) a MiMe allele at one or more REC8 loci, each independently comprising a polynucleotide sequence selected from the group consisting of SEQ ID NOs: 488 and 489; and/or c) a MiMe allele at one or more SPO11-1 loci, each independently comprising a polynucleotide sequence selected from the group consisting of SEQ ID NOs: 490 and 491. In certain embodiments, the subpopulation of genetically uniform polyploid potato seed comprises a) a MiMe allele at one or more REC8 loci, each independently comprising a polynucleotide sequence selected from the group consisting of SEQ ID NOs: 493 and 494; and/or b) a MiMe allele at one or more SPO11-1 loci, each independently comprising a polynucleotide sequence selected from the group consisting of SEQ ID NOs: 495 and 496. In some embodiments, the population of polyploid seed is from an Arabidopsis plant, and the subpopulation of genetically uniform polyploid Arabidopsis seed comprises one or more polynucleotide sequences selected from the group consisting of SEQ ID NOs: 452-463. In certain embodiments, the subpopulation of genetically uniform polyploid Arabidopsis seed comprises a) a MiMe allele at one or more OSD1 loci, each independently comprising a polynucleotide sequence selected from the group consisting of SEQ ID NOs: 452 and 453; b) a MiMe allele at one or more REC8 loci, each independently comprising a polynucleotide sequence selected from the group consisting of SEQ ID NOs: 454 and 455; and/or c) a MiMe allele at one or more SPO11-1 loci, each independently comprising a polynucleotide sequence selected from the group consisting of SEQ ID NOs: 456 and 457. In certain embodiments, the subpopulation of genetically uniform polyploid Arabidopsis seed comprises a) a MiMe allele at one or more OSD1 loci, each independently comprising a polynucleotide sequence selected from the group consisting of SEQ ID NOs: 458 and 459; b) a MiMe allele at one or more REC8 loci, each independently comprising a polynucleotide sequence selected from the group consisting of SEQ ID NOs: 460 and 461; and/or ci a MiMe allele at one or more PAIR1 loci, each independently comprising a polynucleotide sequence selected from the group consisting of SEQ ID NOs: 462 and 463. In some variations, each the one or more OSD1 loci, each of the one or more REC8 loci each of the one or more PAIR1 loci, and/or each of the one or more SP10-14 loci are present on a different homologous chromosome.

In some embodiments, provided herein is a population of polyploid potato seed comprising a subpopulation of genetically uniform polyploid potato seed in an amount of at least 50% of the total number of seeds, the genetically uniform polyploid potato seed comprising three or more haplotypes of the same or related species of plant. In certain embodiments, the population of potato seed was obtained from a single plant or a set of plants (e.g., a set of genetically uniform plants, e.g., a set of genetically uniform F1 hybrids). In certain embodiments, the subpopulation of genetically uniform polyploid potato seed is tetraploid. In certain embodiments, the population of polyploid potato seed has an average pairwise genetic uniformity of at least 90% (e.g., at least about 90%, at least about 91%, at least about 92%, at least about 94%, or at least about 95%) as measured by the Jaccard similarity coefficient. In certain embodiments, the population of polyploid potato seed comprises the subpopulation of genetically uniform polyploid potato seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid potato seed has a pairwise identity of at least about 95% (e.g., at least about 95%, at least about 96%, at least about 97%, at least about 98%, at least about 99%, or about 100%) as measured by the Jaccard similarity coefficient. The subpopulation of genetically uniform polyploid potato seed may have any complete MiMe genotype, partial MiMe genotype, or partially-complemented MiMe genotype described herein. In certain embodiments, the subpopulation of genetically uniform polyploid potato seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid potato seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In certain embodiments, the subpopulation of genetically uniform polyploid potato seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid potato seed has a partial MiMe genotype comprising (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid potato seed has a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid potato seed has a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid potato seed has a partially-complemented MiMe genotype comprising (a) only MiMe alleles at one or more MiMe loci of a first Mime component, wherein the first Mime component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations, the one or more MiMe loci of the component of sister chromatid cohesion during the first division of meiosis comprise REC8; the one or more MiMe loci of the component of DNA double strand breakage during meiotic recombination comprise PAIR1, SPO11-1, SPO11-2, or any combination thereof; the one or more MiMe loci of the component of progression through the second division of meiosis comprise OSD1, CYCA1, or a combination thereof; and/or the one or more MiMe loci of the component of progression through the first division of meiosis comprise PS1. In certain embodiments, germination of a seed of the subpopulation of genetically uniform polyploid potato seed results in a sterile plant that produces inviable gametes, seedless fruit, or a combination thereof. In some embodiments, the subpopulation of genetically uniform polyploid seed comprises one or more polynucleotide sequences selected from the group consisting of SEQ ID NOs: 464-496. In some embodiments, the population of polyploid seed is from a maize plant, and the subpopulation of genetically uniform polyploid maize seed comprises one or more polynucleotide sequences selected from the group consisting of SEQ ID NOs: 448-451. In certain embodiments, the subpopulation of genetically uniform polyploid maize seed comprises a) a MiMe allele at one or more OSD2 loci, each independently comprising a polynucleotide sequence selected from the group consisting or SEQ TD NOs: 448 and 449; b) a MiMe allele at 3 REC8 locus comprising the polynucleotide sequence of SEQ ID NO: 450; and/or c) a MiMe allele at a SPO11-1 locus comprising the polynucleotide sequence of SEQ ID NO: 451. In some embodiments, the population of polyploid seed is from a potato plant, and the subpopulation of genetically uniform polyploid potato seed comprises one or more polynucleotide sequences selected from the group consisting of SEQ ID NOs: 464-496. In certain embodiments, the subpopulation of genetically uniform polyploid potato seed comprises a MiMe allele at one or more SPO11-1 loci, each independently comprising a polynucleotide sequence selected from the group consisting of SEQ ID NOs: 464-466. In certain embodiments, the subpopulation of genetically uniform polyploid potato seed comprises a) a MiMe allele at one or more REC8 loci, each independently comprising a polynucleotide sequence selected from the group consisting of SEQ ID NOs: 468 and 469; and/or b) a MiMe allele at one or more SPO11-1 loci, each independently comprising a polynucleotide sequence selected from the group consisting of SEQ ID NOs: 470 and 471. In certain embodiments, the subpopulation of genetically uniform polyploid potato seed comprises a) a MiMe allele at one or more REC8 loci, each independently comprising: a polynucleotide sequence selected from the group consisting of SEQ ID NOs: 474 and 475; and/or c) a MiMe allele at one or more SPO11-1 loci, each independently comprising a polynucleotide sequence selected from the group consisting of SEQ ID NOs: 476 and 477. In certain embodiments, the subpopulation of genetically uniform polyploid potato seed comprises a MiMe allele at one or more SPO11-1 loci, each independently comprising a polynucleotide sequence selected from the group consisting of SEQ ID NOs: 478 and 479. In certain embodiments, the subpopulation of genetically uniform polyploid potato seed comprises a) a MiMe allele at one or more CYCA1 loci, each independently comprising a polynucleotide sequence selected from the group consisting of SEQ ID NOs: 480 and 481; b) a MiMe allele at one or more REC8 loci, each independently comprising a polynucleotide sequence selected from the group consisting of SEQ ID NOs: 482 and 433; and/or c) a MiMe allele at one or more SPO11-1 loci, each independently comprising a polynucleotide sequence selected from the group consisting of SEQ ID NOs: 484 and 485 In certain embodiments, the subpopulation of genetically uniform polyploid potato seed comprises a) a MiMe allele at one or more CYCA1 loci, each independently comprising a polynucleotide sequence selected from the group consisting of SEQ ID NOs: 486 and 487; b 1a MiMe allele at one or more REC8 loci, each independently comprising a polynucleotide sequence selected from the group consisting of SEQ 11 NOs: 488 and 489; and/or c) a MiMe allele at one or more SPO11-1 loci, each independently comprising a polynucleotide sequence selected from the group consisting of SEQ 11 NOs: 490 and 491 In certain embodiments, the subpopulation of genetically uniform polyploid potato seed comprises a) a MiMe allele at one or more REC8 loci, each independently comprising a polynucleotide sequence selected from the group consisting of SEQ ID NOs: 493 and 494; and/or b) a MiMe allele at one or more SPO11-1 loci, each independently comprising a polynucleotide sequence selected from the group consisting of SEQ ID NOs: 495 and 496. In some embodiments, the population of polyploid seed is from an Arabidopsis plant, and the subpopulation of genetically uniform polyploid Arabidopsis seed comprises one or more polynucleotide sequences selected from the group consisting of SEQ ID NOs: 452-463. In certain embodiments, the subpopulation of genetically uniform polyploid Arabidopsis seed comprises a) a MiMe allele at one or more OSD loci, each independently comprising a polynucleotide sequence selected from the group consisting of SEQ ID NOs: 452 and 453; b)) a MiMe allele at one or more REC8 loci, each independently comprising a polynucleotide sequence selected from the group consisting of SEQ ID NOs: 454 and 455; and/or c) 3 MiMe allele at one or more SPO11-1 loci, each independently comprising a polynucleotide sequence selected from the group consisting of SEQ ID NOs: 456 and 457. In certain embodiments, the subpopulation of genetically uniform polyploid Arabidopsis seed comprises a) a MiMe allele at one or more OSD1 loci, each independently comprising a polynucleotide sequence selected fro-m the group consisting of SEQ ID NOs: 458 and 459; b) a MiMe allele at one or more REC8 loci, each independently comprising a polynucleotide sequence selected from the group consisting of SEQ ID NOs: 460 and 461; and/or c) a MiMe allele at one or more PAIR1 loci, each independently comprising a polynucleotide sequence selected from the group consisting of SEQ ID NOs: 462 and 463. In some variations, each the one or more OSD1 loci, each of the one or more REC8 loci, the one or more PAR1 loci, and/or each of the one or more SPO1-1 loci are present on a different homologous chromosome.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes of the same or related species of plant. In certain embodiments, the population of maize seed was obtained from a single plant or a set of plants (e.g., a set of genetically uniform plants, e.g., a set of genetically uniform F1 hybrids). In certain embodiments, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, or at least about 95%) as measured by the Jaccard similarity coefficient. In one embodiment, the population of polyploid maize seed has an average pairwise genetic uniformity of at least 99% or about 100% as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient. In one embodiment, the population of polyploid maize seed comprises the subpopulation of genetically uniform polyploid maize seed in an amount of at least about 98% of the total number of seeds, wherein each pair of seeds in the subpopulation of genetically uniform polyploid maize seed has a pairwise identity of 100%. The subpopulation of genetically uniform polyploid maize seed may have any complete MiMe genotype, partial MiMe genotype, or partially-complemented MiMe genotype described herein. In certain embodiments, the subpopulation of genetically uniform polyploid maize seed has a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In certain embodiments, the subpopulation of genetically uniform polyploid maize seed has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a partial MiMe genotype comprising (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid maize seed has a partially-complemented MiMe genotype comprising (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In certain embodiments, the subpopulation of genetically uniform polyploid maize seed has a partially-complemented MiMe genotype comprising (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In certain embodiments, the subpopulation of genetically uniform polyploid maize seed has a partially-complemented MiMe genotype comprising (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations, the one or more MiMe loci of the component of sister chromatid cohesion during the first division of meiosis comprise REC8; the one or more MiMe loci of the component of DNA double strand breakage during meiotic recombination comprise PAIR1, SPO11-1, SPO11-2, or any combination thereof; the one or more MiMe loci of the component of progression through the second division of meiosis comprise OSD1, CYCA1, or a combination thereof; and/or the one or more MiMe loci of the component of progression through the first division of meiosis comprise PS1. In certain embodiments, germination of a seed of the subpopulation of genetically uniform polyploid maize seed results in a sterile plant that produces inviable gametes, seedless fruit, or a combination thereof. In some embodiments, the subpopulation of genetically uniform polyploid maize seed comprises one or more polynucleotide sequences selected from the group consisting of SEQ ID NOs: 448-451. In one variation, the subpopulation of genetically uniform polyploid maize seed comprises each of the polynucleotide sequences of SEQ ID NOs: 448-451.

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494. In some variations, the population of polyploid seed is from a maize plant or a set of maize plants (e.g., a set of genetically uniform maize plants, e.g. a set of genetically uniform F1 hybrid maize plants), optionally wherein the one or more OSD1 loci comprise OSD1-1, OSD1-2, and/or OSD1-3, and/or the one or more PAIR1 loci comprise PAIR1-1 and/or PAIR1-2. In certain variations, the population of polyploid seed is from a maize plant or a set of maize plants (e.g., a set of genetically uniform maize plants, e.g. a set of genetically uniform F1 hybrid maize plants) and the one or more OSD1 loci comprise OSD1-1 and OSD1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) one or more osd1 alleles, each comprising a polynucleotide sequence selected from the group consisting of SEQ ID NOS: 448 and 449. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more pair1 alleles, each comprising a polynucleotide sequence selected from the group consisting of SEQ ID NOs: 461, 462, and 463. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more OSD1 loci comprise OSD1-1 and/or OSD1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises one or more rec8 alleles, each comprising a polynucleotide sequence selected from the group consisting of SEQ ID NOs: 465, 466, 468, 469, 474, 475, 482, 483, 488, 489, 493, and 494. In some variations, the population of polyploid seed is from a maize plant or a set of maize plants (e.g., a set of genetically uniform maize plants, e.g. a set of genetically uniform F1 hybrid maize plants), optionally wherein the one or more OSD1 loci comprise OSD1-1, OSD1-2, and/or OSD1-3, and/or the one or more PAIR1 loci comprise PAIR1-1 and/or PAIR1-2. In certain variations, the population of polyploid seed is from a maize plant or a set of maize plants (e.g., a set of genetically uniform maize plants, e.g. a set of genetically uniform F1 hybrid maize plants) and the one or more OSD1 loci comprise OSD1-1 and OSD1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) one or more osd1 alleles, each comprising a polynucleotide sequence selected from the group consisting of SEQ ID NOS: 448 and 449. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more pair1 alleles, each comprising a polynucleotide sequence selected from the group consisting of SEQ ID NOs: 461, 462, and 463. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more OSD1 loci comprise OSD1-1 and/or OSD1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or a set of maize plants (e.g., a set of genetically uniform maize plants, e.g. a set of genetically uniform F1 hybrid maize plants), optionally wherein the one or more OSD1 loci comprise OSD1-1, OSD1-2, and/or OSD1-3. In certain variations, the population of polyploid seed is from a maize plant or a set of maize plants (e.g., a set of genetically uniform maize plants, e.g. a set of genetically uniform F1 hybrid maize plants) and the one or more OSD1 loci comprise OSD1-1 and OSD1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; b) one or more osd1 alleles, each comprising a polynucleotide sequence selected from the group consisting of SEQ ID NOS: 448 and 449; and/or c) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more OSD1 loci comprise OSD1-1 and/or OSD1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or a set of maize plants (e.g., a set of genetically uniform maize plants, e.g. a set of genetically uniform F1 hybrid maize plants), optionally wherein the one or more OSD1 loci comprise OSD1-1, OSD1-2, and/or OSD1-3. In certain variations, the population of polyploid seed is from a maize plant or a set of maize plants (e.g., a set of genetically uniform maize plants, e.g. a set of genetically uniform F1 hybrid maize plants) and the one or more OSD1 loci comprise OSD1-1 and OSD1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; b) one or more osd1 alleles, each comprising a polynucleotide sequence selected from the group consisting of SEQ ID NOS: 448 and 449; and/or c) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more OSD1 loci comprise OSD1-1 and/or OSD1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the one or more CYCA1 loci comprise CYCA1-1, CYCA1-2, and/or CYCA1-3. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; b) one or more cyca1 alleles, each comprising a polynucleotide sequence selected from the group consisting of SEQ ID NOs: 480, 481, 486, and 487; and c) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the one or more CYCA1 loci comprise CYCA1-1, CYCA1-2, and/or CYCA1-3. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; b) one or more cyca1 alleles, each comprising a polynucleotide sequence selected from the group consisting of SEQ ID NOs: 480, 481, 486, and 487; and c) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the one or more CYCA1 loci comprise CYCA1-1, CYCA1-2, and/or CYCA1-3. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more cyca1 alleles, each comprising a polynucleotide sequence selected from the group consisting of SEQ ID NOs: 480, 481, 486, and 487. In some variations, the population of polyploid seed is from a maize plant or a set of maize plants (e.g., a set of genetically uniform maize plants, e.g. a set of genetically uniform F1 hybrid maize plants), optionally wherein the one or more PAIR1 loci comprise PAIR1-1 and/or PAIR1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a rec8 allele comprising the polynucleotide sequence of SEQ ID NO: 450. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the one or more CYCA1 loci comprise CYCA1-1, CYCA1-2, and/or CYCA1-3. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more cyca1 alleles, each comprising a polynucleotide sequence selected from the group consisting of SEQ ID NOs: 480, 481, 486, and 487. In some variations, the population of polyploid seed is from a maize plant or a set of maize plants (e.g., a set of genetically uniform maize plants, e.g. a set of genetically uniform F1 hybrid maize plants), optionally wherein the one or more PAIR1 loci comprise PAIR1-1 and/or PAIR1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a rec8 allele comprising the polynucleotide sequence of SEQ ID NO: 450. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more TDM1 loci comprise TDM1-1 and/or TDM1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more TDM1 loci comprise TDM1-1 and/or TDM1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises one or more rec8 alleles, each comprising a polynucleotide sequence selected from the group consisting of SEQ ID NOs: 465, 466, 468, 469, 474, 475, 482, 483, 488, 489, 493, and 494. In some variations, the population of polyploid seed is from a maize plant or a set of maize plants (e.g., a set of genetically uniform maize plants, e.g. a set of genetically uniform F1 hybrid maize plants), optionally wherein the one or more PAIR1 loci comprise PAIR1-1 and/or PAIR1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a rec8 allele comprising the polynucleotide sequence of SEQ ID NO: 450. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more TDM1 loci comprise TDM1-1 and/or TDM1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises one or more rec8 alleles, each comprising a polynucleotide sequence selected from the group consisting of SEQ ID NOs: 465, 466, 468, 469, 474, 475, 482, 483, 488, 489, 493, and 494. In some variations, the population of polyploid seed is from a maize plant or a set of maize plants (e.g., a set of genetically uniform maize plants, e.g. a set of genetically uniform F1 hybrid maize plants), optionally wherein the one or more PAIR1 loci comprise PAIR1-1 and/or PAIR1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a rec8 allele comprising the polynucleotide sequence of SEQ ID NO: 450. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more TDM1 loci comprise TDM1-1 and/or TDM1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) an os allele, wherein the subpopulation of genetically uniform polyploid seed is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) an os allele, wherein the subpopulation of genetically uniform polyploid seed is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a complete MiMe genotype comprising (i) a ps allele, wherein the subpopulation of genetically uniform polyploid seed is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partial MiMe genotype comprising (i) a ps allele, wherein the subpopulation of genetically uniform polyploid seed is heterozygous for the ps allele, and (ii) at least one Mime allele and at least one non-Mime allele at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the Mime alleles at the SPO11-1 locus comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or a set of maize plants (e.g., a set of genetically uniform maize plants, e.g. a set of genetically uniform F1 hybrid maize plants), optionally wherein the one or more OSD1 loci comprise OSD1-1, OSD1-2, and/or OSD1-3, and/or the one or more PAIR1 loci comprise PAIR1-1 and/or PAIR1-2. In certain variations, the population of polyploid seed is from a maize plant or a set of maize plants (e.g., a set of genetically uniform maize plants, e.g. a set of genetically uniform F1 hybrid maize plants) and the one or more OSD1 loci comprise OSD1-1 and OSD1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; b) one or more osd1 alleles, each comprising a polynucleotide sequence selected from the group consisting of SEQ ID NOS: 448 and 449; and/or c) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; c) one or more pair1 alleles, each comprising a polynucleotide sequence selected from the group consisting of SEQ ID NOs: 461, 462, and 463; and/or d) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more OSD1 loci comprise OSD1-1 and/or OSD1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the one or more CYCA1 loci comprise CYCA1-1, CYCA1-2, and/or CYCA1-3. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; b) one or more cyca1 alleles, each comprising a polynucleotide sequence selected from the group consisting of SEQ ID NOs: 480, 481, 486, and 487; and/or c) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or a set of maize plants (e.g., a set of genetically uniform maize plants, e.g. a set of genetically uniform F1 hybrid maize plants), optionally wherein the one or more PAIR1 loci comprise PAIR1-1 and/or PAIR1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; b) one or more pair1 alleles, each comprising a polynucleotide sequence selected from the group consisting of SEQ ID NOs: 461, 462, and 463; and/or c) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or a set of maize plants (e.g., a set of genetically uniform maize plants, e.g. a set of genetically uniform F1 hybrid maize plants), optionally wherein the one or more PAIR1 loci comprise PAIR1-1 and/or PAIR1-2. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; b) one or more pair1 alleles, each comprising a polynucleotide sequence selected from the group consisting of SEQ ID NOs: 461, 462, and 463; and/or c) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants), optionally wherein the one or more TDM1 loci comprise TDM1-1 and/or TDM1-2. In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partially complemented MiMe genotype comprising (i) a ps allele, wherein the subpopulation of genetically uniform polyploid seed is heterozygous for the ps allele, (ii) an os allele, wherein the subpopulation of genetically uniform polyploid seed is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or a set of maize plants (e.g., a set of genetically uniform maize plants, e.g. a set of genetically uniform F1 hybrid maize plants). In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a soybean plant or a set of soybean plants (e.g., a set of genetically uniform soybean plants, e.g., a set of genetically uniform F1 hybrid soybean plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and a partially complemented MiMe genotype comprising (i) an os allele, wherein the subpopulation of genetically uniform polyploid seed is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), optionally wherein the one or more CYCA1 loci comprise CYCA1-1, CYCA1-2, and/or CYCA1-3. In some variations, the population of polyploid seed is from a potato plant or a set of potato plants (e.g., a set of genetically uniform potato plants, e.g. a set of genetically uniform F1 hybrid potato plants), optionally wherein the subpopulation of genetically uniform polyploid seed comprises a) one or more rec8 alleles, each comprising a polynucleotide sequence selected from the group consisting of SEQ ID NOs: 465, 466, 468, 469, 474, 475, 482, 483, 488, 489, 493, and 494; b) one or more cyca1 alleles, each comprising a polynucleotide sequence selected from the group consisting of SEQ ID NOs: 480, 481, 486, and 487; and/or c) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the population of polyploid seed is from a maize plant or set of maize plants (e.g., a set of genetically uniform maize plants, e.g. a set of genetically uniform F1 hybrid maize plants), optionally wherein the subpopulation of genetically uniform polyploid seed comprises a) a rec8 allele comprising the polynucleotide sequence of SEQ ID NO: 450; and/or b) a spo11-1 allele comprising the polynucleotide sequence of SEQ ID NO: 451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), optionally wherein the subpopulation of genetically uniform polyploid seed comprises a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the population of polyploid seed is from a tomato plant or a set of tomato plants (e.g., a set of genetically uniform tomato plants, e.g., a set of genetically uniform F1 hybrid tomato plants). In some variations, the population of polyploid seed is from a banana plant or a set of banana plants (e.g., a set of genetically uniform banana plants, e.g., a set of genetically uniform F1 hybrid banana plants). In some variations, the population of polyploid seed is from a blueberry plant or a set of blueberry plants (e.g., a set of genetically uniform blueberry plants, e.g., a set of genetically uniform F1 hybrid blueberry plants). In some variations, the population of polyploid seed is from a blackberry plant or a set of blackberry plants (e.g., a set of genetically uniform blackberry plants, e.g., a set of genetically uniform F1 hybrid blackberry plants).

In some embodiments, provided herein is a population of polyploid seed comprising a subpopulation of genetically uniform polyploid seed in an amount of at least 50% of the total number of seeds, the genetically uniform polyploid seed comprising three or more haplotypes of the same or related species of plant and one or more polynucleotide sequences selected from the group consisting of SEQ ID NOs: 448-466, 468-471, 474-491, and 493-496. In certain embodiments, the population was obtained from a single plant or a set of plants (e.g., a set of genetically uniform plants, e.g., a set of genetically uniform F1 hybrids). In certain embodiments, the subpopulation of genetically uniform polyploid seed is triploid, tetraploid, or pentaploid. In one embodiment, the subpopulation of genetically uniform polyploid seed is tetraploid. In certain embodiments, the population of polyploid seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid seed comprises the subpopulation of genetically uniform polyploid seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of seeds, wherein each pair of seeds in the subpopulation of genetically uniform polyploid seed has a pairwise identity of at least about 95% (e.g., at least about 95%, at least about 96%, at least about 97%, at least about 98%, at least about 99%, or about 100%) as measured by the Jaccard similarity coefficient. In some variations, the population of polyploid seed is from a potato plant or a set of potato plants (e.g., a set of genetically uniform potato plants, e.g. a set of genetically uniform F1 hybrid potato plants), wherein the subpopulation of genetically uniform polyploid seed comprises one or more polynucleotide sequences selected from the group consisting of SEQ ID NOs: 464-496. In some variations, the population of polyploid seed is from a maize plant or set of maize plants (e.g., a set of genetically uniform maize plants, e.g. a set of genetically uniform F1 hybrid maize plants), wherein the subpopulation of genetically uniform polyploid seed comprises one or more polynucleotide sequences selected from the group consisting of SEQ ID NOs: 448-451. In some variations, the population of polyploid seed is from an Arabidopsis plant or a set of Arabidopsis plants (e.g., a set of genetically uniform Arabidopsis plants, e.g., a set of genetically uniform F1 hybrid Arabidopsis plants), wherein the subpopulation of genetically uniform polyploid seed comprises one or more polynucleotide sequences selected from the group consisting of SEQ ID NOs: 452-463.

In some embodiments, provided herein is a population of polyploid potato seed comprising a subpopulation of genetically uniform polyploid potato seed in an amount of at least 50% of the total number of seeds, the genetically uniform polyploid potato seed comprising three or more haplotypes and one or more polynucleotide sequences selected from the group consisting of SEQ ID NOs: 464-496. In certain embodiments, the population was obtained from a single potato plant or a set of potato plants (e.g., a set of genetically uniform potato plants, e.g., a set of genetically uniform potato F1 hybrids). In certain embodiments, the subpopulation of genetically uniform polyploid potato seed is tetraploid. In certain embodiments, the population of polyploid potato seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid potato seed comprises the subpopulation of genetically uniform polyploid potato seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of potato seeds, wherein each pair of potato seeds in the subpopulation of genetically uniform polyploid potato seed has a pairwise identity of at least about 95% (e.g., at least about 95%, at least about 96%, at least about 97%, at least about 98%, at least about 99%, or about 100%) as measured by the Jaccard similarity coefficient.

In some embodiments, provided herein is a population of polyploid maize seed comprising a subpopulation of genetically uniform polyploid maize seed in an amount of at least 50% of the total number of seeds, the genetically uniform polyploid maize seed comprising three or more haplotypes and one or more polynucleotide sequences selected from the group consisting of SEQ ID NOs: 448-451. In certain embodiments, the population was obtained from a single maize plant or a set of maize plants (e.g., a set of genetically uniform maize plants, e.g., a set of genetically uniform F1 maize hybrids). In certain embodiments, the subpopulation of genetically uniform polyploid maize seed is tetraploid. In certain embodiments, the population of polyploid maize seed has an average pairwise genetic uniformity of at least 90% (e.g., at least about 90%, at least about 91%, at least about 92%, at least about 94%, at least about 95%, at least about 96%, at least about 97%, at least about 98%, at least about 99%, or about 100%) as measured by the Jaccard similarity coefficient. In certain embodiments, the population of polyploid maize seed comprises the subpopulation of genetically uniform polyploid maize seed in an amount of at least 50% (e.g., at least 50%, at least 60%, at least 70%, at least 75%, at least 80%, at least 85%, at least 90%, at least 95%, at least 96%, at least 97%, at least 98%, or at least 99%) of the total number of maize seeds, wherein each pair of maize seeds in the subpopulation of genetically uniform polyploid maize seed has a pairwise identity of at least about 95% (e.g., at least about 95%, at least about 96%, at least about 97%, at least about 98%, at least about 99%, or about 100%) as measured by the Jaccard similarity coefficient.

### Methods of Producing Populations of Polyploid Seed

In another aspect, provided herein are methods of producing a population of polyploid seed comprising three or more haplotypes wherein at least 50% of the population of polyploid seed are genetically uniform. In some embodiments, the population of polyploid seed comprises a subpopulation of genetically uniform polyploid seed in an amount of at least 50%, the genetically uniform polyploid seed comprising the three or more haplotypes. In some embodiments, the method comprises (a) providing clonal gametes from a pair of parent MiMe plants that together comprise three or more haplotypes; and (b) crossing the clonal gametes to produce the population of polyploid seed. In other embodiments, the method comprises (a) providing clonal gametes from a parent MiMe plant; (b) providing haploid gametes from a homozygous parent non-MiMe plant; and (c) crossing the clonal gametes with the haploid gametes to produce the population of polyploid seed. In other embodiments, the method comprises (a) providing clonal gametes from a parent MiMe plant; (b) providing haploid gametes from a homozygous parent non-MiMe plant; and (c) crossing the clonal gametes with the haploid gametes to produce the population of polyploid seed; wherein the clonal gametes and the haploid gametes together comprise three or more haplotypes. In still other embodiments, the method comprises (a) providing clonal gametes from a parent MiMe plant; (b) providing unreduced, non-clonal gametes from a homozygous parent plant; and (c) crossing the clonal gametes with the unreduced, non-clonal gametes to produce the population of polyploid seed; wherein the clonal gametes and the unreduced, non-clonal gametes together comprise three or more haplotypes.

In another aspect, provided herein are methods of producing a population of polyploid seed comprising a partially-complemented MiMe genotype wherein at least 50% of the population of polyploid seed are genetically uniform. In some embodiments, provided herein are methods of producing a population of polyploid seed comprising a subpopulation of genetically uniform polyploid seed in an amount of at least 50% of the total number of seeds, the subpopulation of genetically uniform seed comprising a partially-complemented MiMe genotype wherein at least 50% of the population of polyploid seed are genetically uniform. In some embodiments, the method comprises (a) providing clonal gametes from a first parent MiMe plant; (b) providing clonal gametes from a second parent MiMe plant; and (c) crossing the clonal gametes to produce the population of polyploid seed comprising a partially-complemented MiMe genotype. In certain embodiments, the polyploid seed (e.g., the subpopulation of genetically uniform seed) comprises one, two, three, or more haplotypes.

In some embodiments, at least 50% of the population of polyploid seed produced are genetically uniform. In some variations, at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid seed produced are genetically uniform. In some embodiments, at least 50% of the population of polyploid seed produced are genetically uniform, wherein the polyploid seed comprises two, three, or more haplotypes. In some variations, at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the population of polyploid seed produced are genetically uniform, wherein the polyploid seed comprises two, three, or more haplotypes.

In some embodiments, the population of polyploid seed comprises a subpopulation of genetically uniform polyploid seed in an amount of at least 50% of the total number of seeds. In some variations, the population of polyploid seed comprises a subpopulation of genetically uniform polyploid seed in an amount of at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the total number of seeds. In some embodiments, the subpopulation of genetically uniform polyploid seed comprises two, three, or more haplotypes. In some variations, the population of polyploid seed comprises a subpopulation of genetically uniform polyploid seed in an amount of at least 60%, least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.99% of the total number of seeds, wherein the genetically uniform polyploid seed comprises two, three, or more haplotypes. In certain variations, the subpopulation of genetically uniform polyploid seed has a complete MiMe genotype, a partial MiMe genotype, or a partially-complemented MiMe genotype.

### Providing Clonal Gametes

In some embodiments, the method of producing a population of polyploid seed comprises providing clonal gametes. In some embodiments, providing clonal gametes comprises growing a plant with a complete MiMe genotype that exhibits a MiMe phenotype and allowing the plant to grow to a reproductive stage until clonal gametes form. In other embodiments, providing clonal gametes comprises generating a plant with a complete MiMe genotype that exhibits a MiMe phenotype and growing said plant to a reproductive stage until clonal gametes form. In some variations, providing clonal gametes further comprises collecting pollen comprising clonal gametes from a plant with a complete MiMe genotype that exhibits a MiMe phenotype.

In some embodiments, the method of producing a population of polyploid seed comprises providing clonal gametes from one or more parent MiMe plants. In some variations, the method of producing a population of polyploid seed comprises providing clonal gametes from a pair of parent MiMe plants. In some embodiments, providing clonal gametes comprises growing one or more parent MiMe plants and allowing them to grow to a reproductive stage until clonal gametes form. In other embodiments, providing clonal gametes comprises generating one or more parent MiMe plants and growing them to a reproductive stage until clonal gametes form. In some variations, providing clonal gametes further comprises collecting pollen comprising clonal gametes from one or more parent MiMe plants. In some additional variations, the pair of parent MiMe plants are diploid, triploid, tetraploid, pentaploid, hexaploid, heptaploid, octoploid, or any combination thereof.

In some embodiments, the method of producing a population of polyploid seed comprises providing clonal gametes from a pair of parent MiMe plants that together comprise three or more haplotypes. In some variations, the pair of parent MiMe plants together comprise three or more haplotypes, four or more haplotypes, five or more haplotypes, six or more haplotypes, seven or more haplotypes, or eight or more haplotypes. In additional variations, the pair of parent MiMe plants together comprise three, four, five, six, seven, or eight haplotypes. In some embodiments, the first parent MiMe plant comprises one or more haplotypes and the second parent MiMe plant comprises one or more haplotypes, wherein the parent MiMe plants together comprise two or more haplotypes. In some embodiments, the first parent MiMe plant comprises one haplotype and the second parent MiMe plant comprises two or more haplotypes, wherein the parent MiMe plants together comprise three or more haplotypes. In other embodiments, each of the parent MiMe plants comprises two or more haplotypes, wherein the parent MiMe plants together comprise four or more haplotypes.

### Parent MiMe Plant Genotypes

In some embodiments, each of the parent MiMe plants has a complete MiMe genotype. In certain embodiments, the complete MiMe genotype comprises one or more genetic modifications resulting in decreased expression of one or more MiMe loci. In other embodiments, the complete MiMe genotype comprises one or more genetic modifications resulting in decreased expression of two or more MiMe loci. In yet another embodiment, the complete MiMe genotype comprises one or more genetic modifications resulting in decreased expression of three or more MiMe loci. In some variations, the MiMe loci may include, but are not limited to, REC8, OSD1, CYCA1, TDM1, PAIR1, SPO11-1, SPO11-2, PRD1, PRD2, DFO, MTOPVIB, DSY1, SY1, SY2, SY3, SY4, SWITCH1/DYAD, PS1, JASON, PC1, PC2, and FC. In one variation, the complete MiMe genotype comprises one or more genetic modifications resulting in decreased expression of REC8. In a second variation, the complete MiMe genotype comprises one or more genetic modifications resulting in decreased expression of OSD1. In a third variation, the complete MiMe genotype comprises one or more genetic modifications resulting in decreased expression of PAIR1. In a fourth variation, the complete MiMe genotype comprises one or more genetic modifications resulting in decreased expression of SPO11-1, SPO11-2, or a combination thereof. In a fifth variation, the complete MiMe genotype comprises one or more genetic modifications resulting in decreased expression of REC8 and SPO11-1. In a sixth variation, the complete MiMe genotype comprises one or more genetic modifications resulting in decreased expression of REC8 and OSD1. In a seventh variation, the complete MiMe genotype comprises one or more genetic modifications resulting in decreased expression of REC8 and PAIR1. In an eighth variation, the complete MiMe genotype comprises one or more genetic modifications resulting in decreased expression of OSD1 and SPO11-1. In a ninth variation, the complete MiMe genotype comprises one or more genetic modifications resulting in decreased expression of OSD1 and PAIR1. In a tenth variation, the complete MiMe genotype comprises one or more genetic modifications resulting in decreased expression of REC8, OSD1, and PAIR1. In an eleventh variation, the complete MiMe genotype comprises one or more genetic modifications resulting in decreased expression of REC8, OSD1, and SPO11-1. In a twelfth variation, the complete MiMe genotype comprises one or more genetic modifications resulting in decreased expression of PS1 and SPO11-1. In a thirteenth variation, the complete MiMe genotype comprises one or more genetic modifications resulting in decreased expression of PS1 and SY3. The complete MiMe genotype may comprise one or more genetic modifications resulting in decreased expression of any combination of MiMe loci described herein or known in the art, wherein a plant that has the complete MiMe genotype exhibits a MiMe phenotype. In some embodiments, the complete MiMe genotype may comprise one or more genetic modifications resulting in non-expression of any combination of MiMe loci described here or known in the art wherein a plant that has the complete MiMe genotype exhibits a MiMe phenotype. In further embodiments, the complete MiMe genotype may comprise one or more genetic modifications resulting in decreased expression, non-expression, or a combination thereof of any combination of MiMe loci described here or known in the art, wherein a plant that has the complete MiMe genotype exhibits a MiMe phenotype. The parent MiMe plant may have any complete MiMe genotype known in the art or described herein, including, but not limited to, complete MiMe genotypes comprising MiMe alleles resulting in decreased expression of any of the MiMe loci described herein. Specific examples of complete MiMe genotypes are shown in Table 10.

In some embodiments, the complete MiMe genotype comprises one or more genetic modifications resulting in decreased expression of REC8, SWITCH1/DYAD, or a combination thereof. In some variations, the complete MiMe genotype comprises one or more genetic modifications resulting in decreased expression of REC8, SWITCH1/DYAD, or a combination thereof and one or more genetic modifications resulting in decreased expression of one or more additional MiMe loci. In additional variations, the complete MiMe genotype comprises one or more genetic modifications resulting in decreased expression of REC8, SWITCH1/DYAD, or a combination thereof and one or more genetic modifications resulting in decreased expression of one or more additional MiMe loci, including, but not limited to, OSD1, CYCA1, TDM1, PC1, PC2, and FC. In yet additional variations, the complete MiMe genotype comprises one or more genetic modifications resulting in decreased expression of REC8, SWITCH1/DYAD, or a combination thereof; one or more genetic modifications resulting in decreased expression of OSD1, CYCA1, TDM1, PC1, PC2, FC, or any combination thereof; and one or more genetic modifications resulting in decreased expression of one or more additional MiMe loci, including, but not limited to, PAIR1, SPO11-1, SPO11-2, PRD1, PRD2, DFO, MTOPVIB, DSY1, SY1, SY2, SY3, and SY4.

In some embodiments, the complete MiMe genotype comprises one or more genetic modifications resulting in decreased expression of one or more MiMe loci including, but not limited to, OSD1, CYCA1, TDM1, PC1, PC2, and FC. In some variations, the complete MiMe genotype comprises one or more genetic modifications resulting in decreased expression of OSD1, CYCA1, TDM1, PC1, PC2, FC, or any combination thereof and further comprises one or more genetic modifications resulting in decreased expression of one or more additional MiMe loci, including, but not limited to, PAIR1, SPO11-1, SPO11-2, PRD1, PRD2, DFO, MTOPVIB, DSY1, SY1, SY2, SY3, and SY4.

In some embodiments, the complete MiMe genotype comprises one or more genetic modifications resulting in decreased expression of one or more MiMe loci including, but not limited to, PAIR1, SPO11-1, SPO11-2, PRD1, PRD2, DFO, MTOPVIB, DSY1, SY1, SY2, SY3, and SY4. In some variations, the complete MiMe genotype comprises one or more genetic modifications resulting in decreased expression of PAIR1, SPO11-1, SPO11-2, PRD1, PRD2, DFO, MTOPVIB, DSY1, SY1, SY2, SY3, SY4, or any combination thereof, and further comprises one or more genetic modifications resulting in decreased expression of one or more additional MiMe loci, which may include, but are not limited to PS1 and JASON.

In some embodiments, the parent MiMe plant has a complete MiMe genotype comprising MiMe alleles conferring decreased expression of MiMe loci of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the parent MiMe plant has a complete MiMe genotype comprising MiMe alleles conferring decreased expression of MiMe loci of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the parent MiMe plant having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes. The parent MiMe plant may have any complete MiMe genotype known in the art or described herein, including, but not limited to, complete MiMe genotypes comprising MiMe alleles resulting in decreased expression of any of the MiMe loci described herein.

### Parent MiMe Plant Complementary Genotypes

In some embodiments, each of the parent MiMe plants has a complete MiMe genotype comprising MiMe alleles conferring decreased expression of MiMe loci of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (b) the second parent MiMe plant has only MiMe alleles at one or more Mime loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; and (c) at least one of the MiMe loci having only MiMe alleles of the first MiMe component of the first parent MiMe plant is the same as at least one of the MiMe loci having only MiMe alleles of the first MiMe component of the second parent MiMe plant. In some variations, at least one of the MiMe loci having only MiMe alleles of the third MiMe component of the first parent MiMe plant is the same as at least one of the MiMe loci having only MiMe alleles of the third MiMe component of the second parent MiMe plant. In other variations, the one or more MiMe loci having only MiMe alleles of the third MiMe component of the first parent MiMe plant are distinct from the one or more MiMe loci having only MiMe alleles of the third MiMe component of the second parent MiMe plant. In some embodiments, (a) the first parent MiMe plant has only MiMe alleles at one or more MiMe loci of a component of sister chromatid cohesion during the first division of meiosis, only MiMe alleles at a first MiMe locus of a component of DNA double strand breakage during meiotic recombination, only non-MiMe alleles at a second MiMe locus of the component of DNA double strand breakage during meiotic recombination, and only MiMe alleles at one or more MiMe loci of a component of progression through the second division of meiosis; (b) the second parent MiMe plant has only MiMe alleles at one or more MiMe loci of the component of sister chromatid cohesion during the first division of meiosis, only non-MiMe alleles at the first MiMe locus of the component of DNA double strand breakage during meiotic recombination, only MiMe alleles at the second MiMe locus of component of DNA double strand breakage during meiotic recombination, and only MiMe alleles at one or more MiMe loci of the of a component of progression through the second division of meiosis; and (c) at least one of the MiMe loci having only MiMe alleles of the component of sister chromatid cohesion during the first division of meiosis of the first parent MiMe plant is the same as at least one of the MiMe loci having only MiMe alleles of the component of sister chromatid cohesion during the first division of meiosis of the second parent MiMe plant. In some variations, at least one of the MiMe loci having only MiMe alleles of the component of progression through the second division of meiosis of the first parent MiMe plant is the same as at least one of the MiMe loci having only MiMe alleles of the component of progression through the second division of meiosis of the second parent MiMe plant. In other variations, the one or more MiMe loci having only MiMe alleles of the component of progression through the second division of meiosis of the first parent MiMe plant are distinct from the one or more MiMe loci having only MiMe alleles of the component of progression through the second division of meiosis of the second parent MiMe plant. Exemplary MiMe loci of each of said MiMe components are extensively described below. In certain embodiments, (a) the first parent MiMe plant has only MiMe alleles at REC8, only MiMe alleles at SPO11-1, only non-MiMe alleles at PAIR1, and only MiMe alleles at OSD1; and (b) the second parent MiMe plant has only MiMe alleles at REC8, only non-MiMe alleles at SPO11-1, only MiMe alleles at PAIR1, and only MiMe alleles at OSD1.

In other embodiments, each of the parent MiMe plants has a complete MiMe genotype comprising MiMe alleles conferring decreased expression of MiMe loci of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In some variations, the parent MiMe plant having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes. In some embodiments, (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component; and (c) at least one of the MiMe loci having only MiMe alleles of the first MiMe component of the first parent MiMe plant is the same as at least one of the MiMe loci having only MiMe alleles of the first MiMe component of the second parent MiMe plant. In certain embodiments, (a) the first parent MiMe plant has only MiMe alleles at one or more MiMe loci of a component of DNA double strand breakage during meiotic recombination, only MiMe alleles at a first MiMe locus of a component of progression through the first division of meiosis, and only non-MiMe alleles at a second MiMe locus of the component of progression through the first division of meiosis; (b) the second parent MiMe plant has only MiMe alleles at one or more MiMe loci of the component of DNA double strand breakage during meiotic recombination, only non-MiMe alleles at the first MiMe locus of the component of progression through the first division of meiosis, and only MiMe alleles at the second MiMe locus of the component of progression through the first division of meiosis; and (c) at least one of the MiMe loci having only MiMe alleles of the component of DNA double strand breakage during meiotic recombination of the first parent MiMe plant is the same as at least one of the MiMe loci having only MiMe alleles of the component of DNA double strand breakage during meiotic recombination of the second parent MiMe plant. In certain embodiments, (a) the first parent MiMe plant has only MiMe alleles at one or more MiMe loci of a component of progression through the first division of meiosis, only MiMe alleles at a first MiMe locus of a the component of DNA double strand breakage during meiotic recombination, and only non-MiMe alleles at a second MiMe locus of the component of DNA double strand breakage during meiotic recombination; (b) the second parent MiMe plant has only MiMe alleles at one or more MiMe loci of the component of progression through the first division of meiosis, only non-MiMe alleles at the first MiMe locus of the component of DNA double strand breakage during meiotic recombination, and only MiMe alleles at the second MiMe locus of the component of DNA double strand breakage during meiotic recombination; and (c) at least one of the MiMe loci having only MiMe alleles of the component of progression through the first division of meiosis of the first parent MiMe plant is the same as at least one of the MiMe loci having only MiMe alleles of the component of progression through the first division of meiosis of the second parent MiMe plant. Exemplary MiMe loci of each of said MiMe components are extensively described below. The parent MiMe plant may have any complete MiMe genotype known in the art or described herein, including, but not limited to, complete MiMe genotypes comprising MiMe alleles resulting in decreased expression of any of the MiMe loci described herein.

In some embodiments, the first parent MiMe plant has a complete MiMe genotype comprising MiMe alleles conferring decreased expression of one or more MiMe loci of a first, second, and third MiMe component, wherein the first MiMe component is (2) a component of DNA double strand breakage during meiotic recombination, the second MiMe component is (1) a component of sister chromatid cohesion during the first division of meiosis and the third MiMe component is (3) a component of progression through the second division of meiosis, and the second parent MiMe plant has a complete MiMe genotype comprising MiMe alleles conferring decreased expression of MiMe loci of the first MiMe component and a fourth MiMe component, wherein the fourth MiMe component is (4) a component of progression through the first division of meiosis. In some variations, the second parent MiMe plant having a complete MiMe genotype comprising MiMe alleles conferring decreased expression of MiMe loci of (2) a component of DNA double strand breakage during meiotic recombination and (4) a component of progression through the first division of meiosis exhibits a MiMe phenotype in male germline cells and/or produces clonal male gametes, and exhibits a wild-type meiosis phenotype in female germline cells and/or produces haploid female gametes. In certain embodiments, (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination; (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of a fourth MiMe component; and (c) at least one of the MiMe loci having only MiMe alleles of the first MiMe component of the first parent MiMe plant is the same as at least one of the MiMe loci having only MiMe alleles of the first MiMe component of the second parent MiMe plant. In some variations, (a) the first parent MiMe plant has only MiMe alleles at one or more MiMe loci of a component is a component of DNA double strand breakage during meiotic recombination, only MiMe alleles one or more MiMe loci of a component of sister chromatid cohesion during the first division of meiosis, only MiMe alleles at one or more MiMe loci of a component of progression through the second division of meiosis, and only non-MiMe alleles at one or more MiMe loci of a component of progression through the first division of meiosis; and (b) the second parent MiMe plant has only MiMe alleles at the one or more MiMe loci of the component of DNA double strand breakage during meiotic recombination, only non-MiMe alleles at the one or more MiMe loci of the component of sister chromatid cohesion during the first division of meiosis, only non-MiMe alleles at the one or more MiMe loci of the component of progression through the second division of meiosis, and only MiMe alleles at the one or more MiMe loci of the component of progression through the first division of meiosis. Exemplary MiMe loci of each of said MiMe components are extensively described below. In one variation, (a) the first parent MiMe plant has only MiMe alleles at SPO11-1, only MiMe alleles at REC8, only MiMe alleles at OSD1, and only non-MiMe alleles at PS1 and JASON; (b) the second parent MiMe plant has only MiMe alleles at SPO11-1, only non-MiMe alleles at REC8, only non-MiMe alleles at OSD1, and only MiMe alleles at PS1 or JASON.

In some embodiments, the first parent MiMe plant, the second parent MiMe plant, or both has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some variations, the parent MiMe plant is a maize plant and the one or more OSD1 loci comprise OSD1-1, OSD1-2, and/or OSD1-3, and/or the one or more PAIR1 loci comprise PAIR1-1 and/or PAIR1-2. In other variations, the parent MiMe plant is a soybean plant and the one or more OSD1 loci comprise OSD1-1 and/or OSD1-2.

In some embodiments, the first parent MiMe plant, the second parent MiMe plant, or both has a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some variations, the parent MiMe plant is a maize plant and the one or more OSD1 loci comprise OSD1-1, OSD1-2, and/or OSD1-3. In other variations, the parent MiMe plant is a soybean plant and the one or more OSD1 loci comprise OSD1-1 and/or OSD1-2.

In some embodiments, the first parent MiMe plant, the second parent MiMe plant, or both has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1.

In some embodiments, the first parent MiMe plant, the second parent MiMe plant, or both has a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some variations, the parent MiMe plant is a maize plant and the one or more PAIR1 loci comprise PAIR1-1 and/or PAIR1-2.

In some embodiments, the first parent MiMe plant, the second parent MiMe plant, or both has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In some variations, the parent MiMe plant is a soybean plant and the one or more TDM1 loci comprise TDM1-1 and/or TDM1-2.

In some embodiments, the first parent MiMe plant, the second parent MiMe plant, or both has a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In some variations, the parent MiMe plant is a soybean plant and the one or more TDM1 loci comprise TDM1-1 and/or TDM1-2. In some variations, the parent MiMe plant is a maize plant and the one or more PAIR1 loci comprise PAIR1-1 and/or PAIR1-2.

In some embodiments, the first parent MiMe plant, the second parent MiMe plant, or both has a complete MiMe genotype comprising (i) an os allele, wherein the first parent MiMe plant, the second parent MiMe plant, or both is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1.

In some embodiments, the first parent MiMe plant, the second parent MiMe plant, or both has a complete MiMe genotype comprising (i) a ps allele, wherein the first parent MiMe plant, the second parent MiMe plant, or both is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1.

In some embodiments, the first parent MiMe plant, the second parent MiMe plant, or both comprise one or more polynucleotide sequences selected from the group consisting of SEQ ID NOs: 448-466, 468-471, 474-491, and 493-496.

### Providing Haploid Gametes

In some embodiments, the method of producing a population of polyploid seed comprises providing haploid gametes from a homozygous parent non-MiMe plant. In some embodiments, providing haploid gametes comprises growing one or more homozygous parent non-MiMe plants and allowing them to grow to a reproductive stage until haploid gametes form. In some variations, providing haploid gametes further comprises collecting haploid pollen from one or more homozygous parent non-MiMe plants. In some additional variations, the one or more homozygous parent non-MiMe plants may be haploid, monoploid, diploid, triploid, tetraploid, pentaploid, hexaploid, heptaploid, octoploid, or any combination thereof. In some embodiments, the homozygous parent non-MiMe plant is diploid and the haploid gametes are monoploid gametes.

In some embodiments, the method of producing a population of polyploid seed comprises (a) providing clonal gametes from a parent MiMe plant; and (b) providing haploid (e.g., monoploid) gametes from a homozygous parent non-MiMe plant, wherein the clonal gametes and the haploid (e.g., monoploid) gametes together comprise three or more haplotypes. In some variations, the clonal gametes and the haploid gametes together comprise three or more haplotypes, four or more haplotypes, five or more haplotypes, six or more haplotypes, seven or more haplotypes, or eight or more haplotypes. In additional variations the clonal gametes and the haploid gametes together comprise three, four, five, six, seven, or eight haplotypes. In some embodiments, the haploid gametes comprise one haplotype and the clonal gametes comprise two or more haplotypes, wherein the clonal gametes and the haploid gametes together comprise three or more haplotypes.

### Providing Unreduced, Non-Clonal Gametes

In some embodiments, the method of producing a population of polyploid seed comprises providing unreduced, non-clonal gametes. In some embodiments, the method of producing a population of polyploid seed comprises providing unreduced, non-clonal gametes from one or more parent plants. In certain embodiments, providing unreduced, non-clonal gametes comprises: growing a parent plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis; and allowing the parent plant to grow to a reproductive stage until unreduced, non-clonal gametes form. In other embodiments, providing unreduced, non-clonal gametes comprises: generating a parent plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis; and growing said parent plant to a reproductive stage until unreduced, non-clonal gametes form. In some variations, providing unreduced, non-clonal gametes further comprises collecting pollen comprising unreduced, non-clonal gametes from a parent plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. The MiMe loci of a of a component of progression through the first division of meiosis may be any loci of a component of progression through the first division of meiosis known in the art or described herein, including, but not limited to, PS1 and JASON. The MiMe loci of a component of a component of progression through the second division of meiosis may be any loci of a component of a component of progression through the second division of meiosis known in the art or described herein, including, but not limited to, OSD1, CYCA1, TDM1, PC1, PC2, and FC. In certain embodiments, the parent plant is a plant of an inbred plant line. In some variations, the parent plant is diploid, triploid, tetraploid, pentaploid, hexaploid, heptaploid, octoploid, or any combination thereof.

### Crossing Clonal Gametes

In some embodiments, the method of producing a population of polyploid seed comprises crossing the clonal gametes to produce the population of polyploid seed. In some variations, crossing the clonal gametes to produce the population of polyploid seed comprises contacting pollen comprising clonal gametes with the stigma of a pistil comprising clonal gametes. In some variations, crossing the clonal gametes to produce the population of polyploid seed comprises contacting pollen comprising clonal gametes of a first plant with the stigma of a pistil comprising clonal gametes of a second plant. In some embodiments, crossing the clonal gametes to produce the population of polyploid seed comprises contacting pollen comprising clonal gametes from a first parent MiMe plant with the stigma of a pistil comprising clonal gametes of a second parent MiMe plant. In some embodiments, the population of polyploid seed is produced by crossing the clonal gametes and allowing seeds to form.

In other embodiments, the method of producing a population of polyploid seed comprises crossing clonal gametes with haploid gametes to produce the population of polyploid seed. In one variation, crossing the clonal gametes with the haploid gametes to produce the population of polyploid seed comprises contacting pollen comprising clonal gametes with the stigma of a pistil comprising haploid gametes. In another variation, crossing the clonal gametes with the haploid gametes to produce the population of polyploid seed comprises contacting pollen comprising haploid gametes with the stigma of a pistil comprising clonal gametes. In some embodiments, crossing the clonal gametes with the haploid gametes to produce the population of polyploid seed comprises contacting pollen comprising clonal gametes from a parent MiMe plant with the stigma of a pistil comprising haploid gametes of a homozygous parent non-MiMe plant. In other embodiments, crossing the clonal gametes with the haploid gametes to produce the population of polyploid seed comprises contacting pollen comprising haploid gametes from a homozygous parent non-MiMe plant with the stigma of a pistil comprising clonal gametes of a parent MiMe plant. In some variations, the homozygous parent non-MiMe plant and the haploid gametes are monoploid gametes. In some embodiments, the population of polyploid seed is produced by crossing the clonal gametes with the haploid gametes and allowing seeds to form.

In yet other embodiments, the method of producing a population of polyploid seed comprises crossing clonal gametes with unreduced, non-clonal gametes to produce the population of polyploid seed. In one variation, crossing the clonal gametes with the unreduced, non-clonal gametes to produce the population of polyploid seed comprises contacting pollen comprising clonal gametes with the stigma of a pistil comprising unreduced, non-clonal gametes. In another variation, crossing the clonal gametes with the unreduced, non-clonal gametes to produce the population of polyploid seed comprises contacting pollen comprising unreduced, non-clonal gametes with the stigma of a pistil comprising clonal gametes. In some embodiments, crossing the clonal gametes with the unreduced, non-clonal gametes to produce the population of polyploid seed comprises contacting pollen comprising clonal gametes from a parent MiMe plant with the stigma of a pistil comprising unreduced, non-clonal gametes of a homozygous parent plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, crossing the clonal gametes with the unreduced, non-clonal gametes to produce the population of polyploid seed comprises contacting the stigma of a pistil comprising clonal gametes from a parent MiMe plant with pollen comprising unreduced, non-clonal gametes of a homozygous parent plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, the population of polyploid seed is produced by crossing the clonal gametes with the unreduced, non-clonal gametes and allowing seeds to form.

In some embodiments, crossing clonal gametes to produce the population of polyploid seed comprises (a) collecting pollen from a first parent MiMe plant having only MiMe alleles at the one or more MiMe loci of the component of DNA double strand breakage during meiotic recombination, only non-MiMe alleles at the one or more MiMe loci of the component of sister chromatid cohesion during the first division of meiosis, only non-MiMe alleles at the one or more MiMe loci of the component of progression through the second division of meiosis, and only MiMe alleles at the one or more MiMe loci of the component of progression through the first division of meiosis; and (b) contacting the pollen from the first parent MiMe plant with a stigma of a second parent MiMe plant having only MiMe alleles at one or more MiMe loci of a component is a component of DNA double strand breakage during meiotic recombination, only MiMe alleles one or more MiMe loci of a component of sister chromatid cohesion during the first division of meiosis, only MiMe alleles at one or more MiMe loci of a component of progression through the second division of meiosis, and only non-MiMe alleles at one or more MiMe loci of a component of progression through the first division of meiosis.

### Methods of Breeding Polyploid Hybrid Plant Lines Comprising Three or More Haplotypes

In yet another aspect, provided herein are methods of breeding a polyploid hybrid plant line comprising three or more haplotypes, comprising obtaining a set of lines of a plant; breeding the lines using traditional plant breeding methods to produce a set of candidate lines of the plant; and selecting two or more candidate lines together comprising three or more haplotypes for crossing. In some embodiments, after the selection of candidate lines, the methods further comprise generating two parent MiMe plants from the two or more candidate lines; providing clonal gametes from each of the parent MiMe plants; and crossing the clonal gametes to produce a hybrid polyploid seed comprising the three or more haplotypes. In alternative embodiments, after the selection of candidate lines, the methods further comprise generating a single parent MiMe plant from one of the two or more candidate lines; providing clonal gametes from the parent MiMe plant; providing haploid (e.g., monoploid) gametes from a homozygous parent non-MiMe plant of one of the two or more candidate lines; and crossing the clonal gametes with the haploid (e.g., monoploid) gametes to produce a hybrid polyploid seed. In yet additional embodiments, after the selection of candidate lines, the methods further comprise generating a single parent MiMe plant from one of the two or more candidate lines; providing clonal gametes from the parent MiMe plant; providing unreduced, non-clonal gametes from homozygous parent plant of one of the two or more candidate lines; and crossing the clonal gametes with the unreduced, non-clonal gametes to produce a hybrid polyploid seed. In some embodiments, after the crossing of the clonal gametes or the crossing of the clonal gametes with the haploid (e.g., monoploid) gametes, or crossing of the clonal gametes with unreduced, non-clonal gametes, the methods further comprise growing the hybrid polyploid seed to produce a hybrid polyploid plant and evaluating one or more characteristics of the hybrid polyploid plant.

In still another aspect, provided herein are methods of breeding hybrid polyploid plants, comprising obtaining a set of lines of a plant; breeding the lines using traditional plant breeding methods to produce a set of candidate lines of the plant; selecting two or more candidate lines for crossing; and generating a first parent MiMe plant and a second parent MiMe plants from the two or more candidate lines. In some embodiments, the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component. In certain embodiments, at least one of the MiMe loci having only MiMe alleles of the first MiMe component of the first parent MiMe plant is the same as at least one of the MiMe loci having only MiMe alleles of the first MiMe component of the second parent MiMe plant. In some embodiments, the first and second parent MiMe plants further have only MiMe alleles at one or more MiMe loci of a third MiMe component, wherein the one or more MiMe loci having only MiMe alleles of the third MiMe component of the first and second parent MiMe plants are the same or different. In certain embodiments, the parent MiMe plants together comprise two, three, or more haplotypes. In some embodiments, the methods further comprise providing clonal gametes from each of the parent MiMe plants; crossing the clonal gametes to produce a polyploid seed; growing the polyploid seed to produce a hybrid polyploid plant; and evaluating one or more characteristics of the hybrid polyploid plant.

### Obtaining Plant Lines

In some embodiments, the method of breeding a polyploid hybrid plant line comprising two, three, or more haplotypes comprises obtaining a set of lines of a plant. In preferred embodiments, the set of lines of a plant is genetically diverse and comprises a large and diverse pool of haplotypes. In some embodiments, the method of breeding a polyploid hybrid plant line comprising two, three, or more haplotypes comprises obtaining a set of lines of the same or related species of plant. The set of lines of the plant may be obtained from any source and by any methods known in the art. In some embodiments, the set of lines is obtained from sources including, but not limited to, natural diversity, existing breeding programs, dihaploid induction of polyploid lines, or any combination thereof.

In some embodiments, the set of plant lines is a genetically diverse founder population of plants. These plants may be collected from existent diploid germplasm sources such as wild species, progenitor species and landraces or from recent diploid inbred line-based breeding efforts (Jansky et al. 2016. Reinventing potato as a diploid inbred line-based crop. Crop Science 56, no. 4: 1412-1422.). In some instances, these plants may have high genetic load and may not have undergone the narrowing of genetic variability attributable to the elite selection practices imposed on modern cultivated materials. As such, many possess traits promoting their fitness within non-agrarian environments in the case of wild progenitors, or have been adapted to vastly differing cultivation practices and agricultural environments in the case of landraces. In either instance, these founding plant materials may contain suites of both desirable and undesirable agronomic characteristics that may be recombined, selected, and complemented to develop a commercially viable product.

In some embodiments, the method of breeding a polyploid hybrid plant line comprising two, three, or more haplotypes comprises obtaining a set of diploid lines of a plant. The diploid lines of the plant may be obtained by any methods known in the art or described herein. In some variations, obtaining a set of diploid lines of a plant comprises dihaploid induction of polyploid lines. New diploid plant germplasm may be created from polyploid sources by one of several ploidy manipulation approaches to reduce the ploidy of a plant's progeny to the diploid level. One of the most frequent and practiced scenarios is the reduction of elite cultivated tetraploid source materials (2n=4x=48) to diploids (2n=2x=24) via ploidy reduction methods such as pseudogamy (a.k.a. gynogenesis or sperm-dependent parthenogenesis). This approach may provide an efficient means to create new diploid plants that are each a sampling of half the alleles of a tetraploid line. As such, these dihaploids are less likely to bear the genetic load of diploid wild progenitor species or landraces and are more apt to complement the germplasm sources attained by other methods described herein. Moreover, undesirable recessive alleles masked at the cultivated tetraploid level may be revealed phenotypically and selected against at the diploid level.

In certain embodiments, the obtaining a set of lines of a plant comprises creating reduced-ploidy plant lines by haploid induction of polyploid plant lines. Haploid induction may be achieved in a variety of plant species by, for example, interspecific hybridization with a haploid inducer, intraspecific hybridization with a haploid inducer, or modification of CENH3 activity or expression (e.g. by RNA interference and/or expression of a modified CENH3 protein). Methods of haploid induction are well known in the literature, and examples are described Wan, et al. (1989. Efficient production of doubled haploid plants through colchicine treatment of anther-derived maize callus. Theor. Appl. Genet., 77:889-892.) and Ren et al. (2017. Novel technologies in doubled haploid line development. Plant Biotechnol J 15, 1361-1370.) and references cited therein. In some variations, the obtaining a set of lines of a plant comprises haploid induction of one or more tetraploid lines to create one or more diploid lines. In other variations, the obtaining a set of lines of a plant comprises haploid induction of hexaploid, octoploid, or higher even-ploidy lines to create one or more lines of half the original ploidy.

### Heterotic Groups

In some embodiments, the method of breeding a polyploid hybrid plant line comprises organizing the set of lines into two, three, or more heterotic groups, wherein each heterotic group comprises a haplotype, and wherein the haplotypes are grouped based on observed or predicted heterotic performance when combined in the hybrid polyploid plant. In some embodiments, the method of breeding a polyploid hybrid plant line comprises organizing the set of lines into four or more heterotic groups. In some variations, the method of breeding a polyploid hybrid plant line comprises organizing the set of lines into five or more heterotic groups, six or more heterotic groups, seven or more heterotic groups, or eight or more heterotic groups.

The obtained set of lines of the plant may be organized by assigning their membership into complementary heterotic groups based upon both their empirically-defined Endosperm Balance Number for the genus in question (Ortiz and Ehlenfeldt. 1992. The importance of Endosperm Balance Number in potato breeding and the evolution of tuber-bearing Solanum species. Euphytica 60, 105-113; Arisumi. 1982. Endosperm balance numbers among New Guinea-Indonesian Impatiens species, Journal of Heredity, 73:3, 240-242; Birchler. 1993. Dosage analysis of maize endosperm development. Annu Rev Genet. 27:181-204; Nishiyama et al. 2007. Embryological studies on cross-incompatibility between 2× and 4× in Brassica, Japanese Journal of Genetics, 41:1, 27-42) and heterotic patterns observed from preliminary estimation or prediction of their combining ability for traits and environments of interest at the diploid and polyploid levels. In this context, the combining ability is an estimation of the value of a plant as a parent as inferred by progeny testing in an established factorial or hierarchical mating design. Combining ability at the polyploid hybrid level is of primary importance; however, sufficient diploid hybrid performance is necessary to permit the diploid hybrid to serve as a parent for seed production. Furthermore, assessment of combining abilities at the simplified diploid level will better inform predictive models of genetic architecture.

In defining heterotic groups, the main goal is identifying subpopulations of the set of lines of the plant based on employment of a clustering procedure that maximizes some measure of interpopulation combining ability. Exhaustive evaluation of all possible parental combinations for the final polyploid hybrid, as in the case of a factorial mating scheme, is infeasible for all but a trivial number of potential parents (i.e. even a partial diallel ignoring reciprocal crosses scales at (n+3)!/(4!(n−1)!) crosses per n parents). Yet, hierarchical mating schemes necessitate an understanding and judgment of which set of “testers” or analogous constructs should serve as a relevant and efficient basis or frame of reference for inferring combining ability. The sample of testers selected invariably biases perceptions of existing heterotic patterns. Furthermore, the relative importance of traits and environments of interest used to infer these combining abilities are dynamic and depend upon market trends. As such, predictive modeling is essential and the process of assigning and refining heterotic groups and testers to represent them is one of iterative improvement and refinement throughout repeated cycles of the breeding process. Nonetheless, once preliminary heterotic group membership is assigned, interpopulation improvement of the diploid germplasm and development of polyploid hybrid plants may proceed.

### Breeding Plant Lines

In some embodiments, the method of breeding a polyploid hybrid plant line comprising three or more haplotypes comprises breeding the lines using traditional plant breeding methods to produce a set of candidate lines of the plant. The method may comprise breeding the lines using any traditional plant breeding method known in the art or described herein. In some embodiments, the breeding of the lines comprises reciprocal recurrent selection. In additional embodiments, the breeding of the lines comprises inbreeding one or more of the lines to homozygosity. In some variations, the breeding of the lines comprises crossing, selfing (self-pollinating), and backcrossing the lines to produce candidate lines. In additional variations, the breeding of the lines comprises crossing pairs of the lines to generate an F1 (first filial) generation, followed by several generations of selfing (generating F2, F3, etc.). In yet additional variations, the breeding of the lines comprises backcrossing (BC) steps, whereby the offspring is backcrossed to one of the parental lines, termed the recurrent parent.

There are numerous steps that may be taken in breeding the lines of the plant using traditional plant breeding methods to produce a set of candidate lines of the plant. The choice of breeding method depends on the mode of plant reproduction and the heritability of the trait(s) being improved. Backcross breeding may be used to transfer one or a few favorable genes for a highly heritable trait into a desirable line. This approach has been used extensively for breeding disease-resistant lines. Various recurrent selection techniques may be used to improve quantitatively inherited traits controlled by numerous genes. The use of recurrent selection in self-pollinating crops depends on the ease of pollination, the frequency of successful hybrids from each pollination, and the number of hybrid offspring from each successful cross. A breeder can initially select and cross two or more parental lines, followed by repeated selfing and selection, producing many new genetic combinations. Moreover, a breeder can generate multiple different genetic combinations by crossing, selfing, generating mutations, or any combination thereof. A plant breeder can then select which lines to select as candidate lines. Recurrent selection techniques are reviewed in Vasal et al. (2004. Population Improvement Strategies for Crop Improvement. In: Plant Breeding. Springer, p 391-406).

The development of candidate lines for the methods described herein may include obtaining parental lines, crossing of these lines, and evaluating the crosses. Pedigree breeding and recurrent selection breeding methods may be used to develop candidate lines from breeding populations. Breeding programs may combine desirable traits from two or more varieties or various broad-based sources into breeding pools from which lines are developed by selfing and selection of desired phenotypes. The new lines may be further crossed with other lines and the hybrids from these crosses may be evaluated for potential selection as candidate lines.

Choice of breeding or selection methods depends on the mode of plant reproduction and the heritability of the trait(s) being improved. For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

In some embodiments, the breeding of the lines comprises inbreeding one or more of the lines to homozygosity. In some variations, inbreeding a plant line to homozygosity may comprise selfing plants of the line for two or more generations, such as for five to seven generations, to produce an inbred or homozygous plant line. Homozygous plant lines may also be developed by the production of double haploids. Double haploids are produced by generating a haploid plant from a heterozygous plant and the doubling of the genome of the haploid plant to produce a completely homozygous individual. The process of generating a haploid plant is also known as haploid induction. Haploid induction can be achieved in a variety of plants using methods well-known in the art and described herein. After a haploid plant is generated, genome doubling may occur spontaneously or may be achieved artificially using, for example, colchicine, amiprophos-mehtyl (APM), oryzalin, pronamide, trifluralin, or nitrous oxide. Methods of producing double haploids are well known in the literature, and examples are described Wan, et al. (1989. Efficient production of doubled haploid plants through colchicine treatment of anther-derived maize callus. Theor. Appl. Genet., 77:889-892.) and Ren et al. (2017. Novel technologies in doubled haploid line development. Plant Biotechnol J 15, 1361-1370.) and references cited therein.

Pedigree breeding is used commonly for the improvement of self-pollinating crops or inbred lines of cross-pollinating crops. Two parents which possess favorable, complementary traits are crossed to produce an F1 population. An F2 population is produced by selfing one or several F1s or by intercrossing two F1s (sib mating). Selection of the best individuals may begin in the F2 population; then, beginning in the F3, the best individuals in the best families may be selected. Replicated testing of families, or hybrid combinations involving individuals of these families, may follow in the F4 generation to improve the effectiveness of selection for traits with low heritability. At an advanced stage of inbreeding (i.e., F6 and F7), the best lines or mixtures of phenotypically similar lines may be tested for potential selection as candidate lines.

Mass and recurrent selections can be used to improve lines of either self- or cross-pollinating crops. A genetically variable population of heterozygous individuals may be either identified or created by intercrossing several different parents. The best plants are selected based on individual superiority, outstanding progeny, or heterotic performance. The selected plants are intercrossed to produce a new population in which further cycles of selection are continued.

Backcross breeding may be used to transfer genes for a simply inherited, highly heritable trait into a desirable homozygous line that is the recurrent parent. The source of the trait to be transferred is called the donor parent. The resulting plant is expected to have the attributes of the recurrent parent and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generational advancement is completed.

In addition to phenotypic observations, the genotype of a plant can also be examined during breeding to produce candidate lines. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs).

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of lines as parental varieties or ancestors of a plant by providing a means of tracking genetic profiles through crosses.

Mutation breeding may also be used in the breeding of plant lines to produce candidate lines. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into an existing line by traditional breeding techniques. Details of mutation breeding can be found in Principles of Cultivar Development by Fehr, Macmillan Publishing Company, 1993.

Additional non-limiting examples of breeding methods that may be used include, without limitation, those found in Allard (1960. Principles of Plant Breeding, John Wiley and Son, pp. 115-161). Simmonds (1979. Principles of Crop Improvement, Longman Group Limited), Sneep (1979. Plant Breeding Perspectives, Unipub), and Fehr and Walt (1987. Principles of Cultivar Development, pp. 261-286).

In certain embodiments, breeding the lines of the plant comprises generating and maintaining one or more plant lines having complete or partial MiMe genotypes. The one or more plant lines having complete or partial MiMe genotypes may be maintained via vegetative propagation, selfing, apomixis, cell culture, or any combination thereof. The MiMe alleles of the one or more plant lines having partial MiMe genotypes may be propagated across breeding cycles to reduce the number of required editing or transgenesis events to introduce partial or complete MiMe genotypes into candidate lines of the plant. In some embodiments, each of the heterotic groups may be further divided into two subgroups, resulting in each heterotic group containing a small subgroup of plant lines having a partial MiMe genotype, maintained through outbreeding and stabilizing selection for the partial MiMe genotype and a wild-type meiosis phenotype, and another larger subgroup of plant lines that do not comprise MiMe alleles. The subgroup containing the plant lines having a partial MiMe genotype may be slower-cycling during breeding, as genotyping and stabilizing selection for the partial MiMe genotype and wild-type meiosis phenotype may be undertaken in some or all progeny. Within this subgroup, a subset of all possible intragroup crosses, which are expected to bear some offspring possessing the partial MiMe genotype and a wild-type meiosis phenotype, is selected and realized by considering the following criteria: 1) per se performance of the plant line, 2) genotype or pedigree-based estimation or prediction of breeding value and combining ability with regard to complementing the alternate subgroup within the heterotic group as well as the other heterotic groups at the polyploid hybrid level, and 3) management of inbreeding with respect to both subgroups of the heterotic group. After genotyping and selecting the subset of realized recombinant diploid progeny having a partial MiMe genotype and a wild-type meiosis phenotype, these plants may serve in the following breeding roles: 1) field evaluations to improve the estimation or prediction of breeding values and combining abilities, 2) testers to aid heterotic group (re)assignment, 3) progenitors in recurrent cycles of diploid intergroup improvement to increase desirable allele frequencies and linkage disequilibria, and/or 4) great-grandparents in tetraploid hybrid plant development.

In some embodiments, breeding the lines of the plant comprises (i) introducing a partial MiMe genotype into one or more lines of the plant within a heterotic group to produce a plant line having a partial MiMe genotype, wherein the plant line having a partial MiMe genotype comprises one or more haplotypes, (ii) crossing the plant line having the partial MiMe genotype with itself or another plant line, and (iii) propagating the plant line having the partial MiMe genotype by selecting for the partial MiMe genotype in the offspring of the cross of step (ii). In some variations, step (ii) comprises crossing the plant line having the partial MiMe genotype with itself. In other variations, step (ii) comprises crossing the plant line having the partial MiMe genotype with another plant within the same heterotic group or a different heterotic group.

In some embodiments, haploid induction may be used to maintain polyploid plant lines having a complete MiMe genotype. In certain embodiments, maintaining a polyploid plant line having a complete MiMe genotype comprises crossing a polyploid plant having a complete MiMe genotype with a haploid inducer to produce progeny of the same ploidy as the plant having the complete MiMe genotype. In one variation, maintaining a polyploid plant line having a complete MiMe genotype comprises crossing a tetraploid plant having a complete MiMe genotype with a haploid inducer to produce tetraploid progeny.

### Selecting Candidate Lines

In some embodiments, the method of breeding a polyploid hybrid plant line comprising two, three, or more haplotypes comprises selecting two or more candidate lines together comprising two, three, or more haplotypes. In some variations, the method of breeding a hybrid polyploid plant comprises selecting two or more candidate lines together comprising three or more haplotypes or four or more haplotypes. In some embodiments, one or more of the candidate lines are inbred lines. In additional embodiments, one or more of the candidate lines are hybrid lines. In certain embodiments, the selecting of candidate lines is guided by the one or more characteristics of a hybrid polyploid plant comprising the two, three, or more haplotypes comprised by the two or more candidate lines. In certain embodiments, the selecting of candidate lines is guided by the one or more characteristics of a hybrid polyploid plant comprising the two or more haplotypes, three or more haplotypes, or four or more haplotypes comprised by the two or more candidate lines. In some variations, the selecting of candidate lines is guided by the observed or predicted heterotic performance of the two or more haplotypes, three or more haplotypes, or four or more haplotypes comprised by the two or more candidate lines.

### Generating Parent MiMe Plants

In some embodiments, the method of breeding a polyploid hybrid plant line comprising two, three, or more haplotypes comprises generating one or more parent MiMe plants from one or more of the two or more candidate lines, wherein the parent MiMe plant has a complete MiMe genotype. In some variations, the method of breeding a polyploid hybrid plant line comprising two, three, or more haplotypes comprises generating two parent MiMe plants from two of the two or more candidate lines, wherein each of the parent MiMe plants has a complete MiMe genotype. In some embodiments, the method of breeding a hybrid polyploid plant comprises generating a first parent MiMe plant from one of the candidate lines and generating a second parent MiMe plant from one of the candidate lines, wherein the parent MiMe plant has a complete MiMe genotype. In some variations, the method of breeding a hybrid polyploid plant comprises generating two parent MiMe plants from two of the two or more candidate lines, wherein the parent MiMe plant has a complete MiMe genotype. In some embodiments, the complete MiMe genotypes of the one or more parent MiMe plants comprise alleles that are naturally-occurring, introduced via genetic modification, or a combination thereof. The one or more parent MiMe plants may have any complete MiMe genotype known in the art or described herein. The generating of the parent MiMe plants may comprise introducing any of the parent MiMe plant genotypes described herein (see “Parent MiMe Plant Genotypes” and “Parent MiMe Plant Complementary Genotypes”).

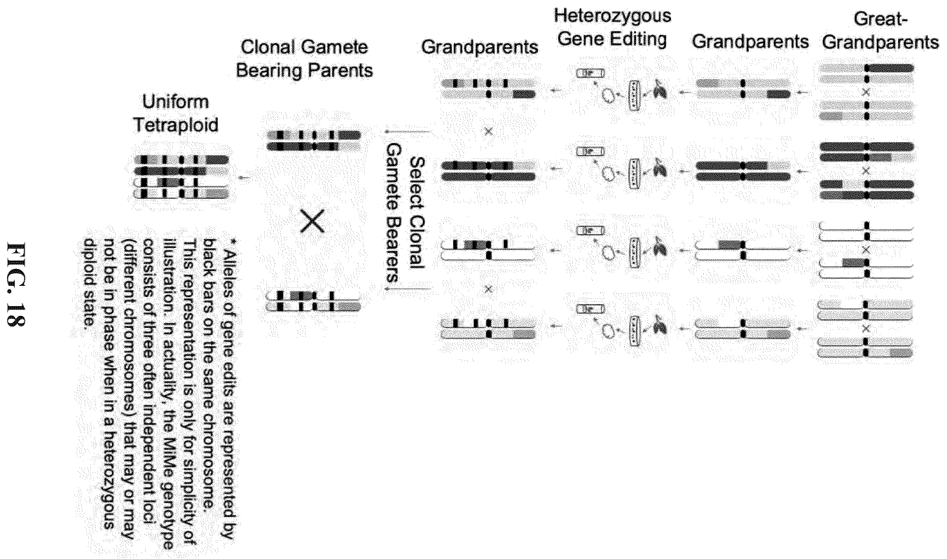

In certain embodiments, the generating of the one or more parent MiMe plants comprises introducing a complete MiMe genotype directly into one or more candidate lines to produce the one or more parent MiMe plants. In some variations, the generating of the two parent MiMe plants comprises introducing a complete MiMe genotype directly into two or more candidate lines to produce the two parent MiMe plants, for example, as shown in FIG. 16, 17, or 23.

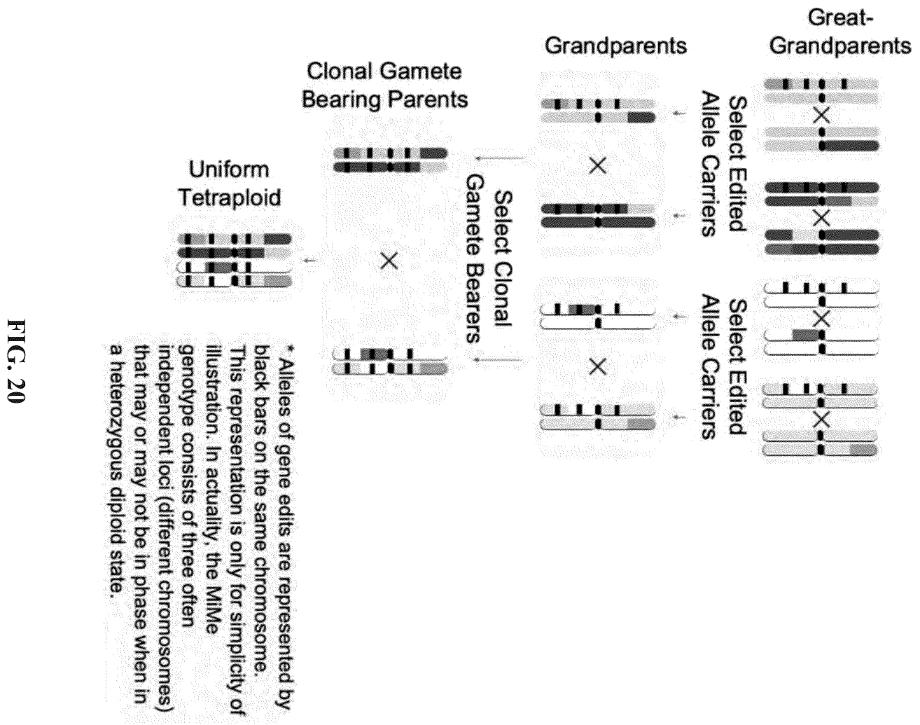

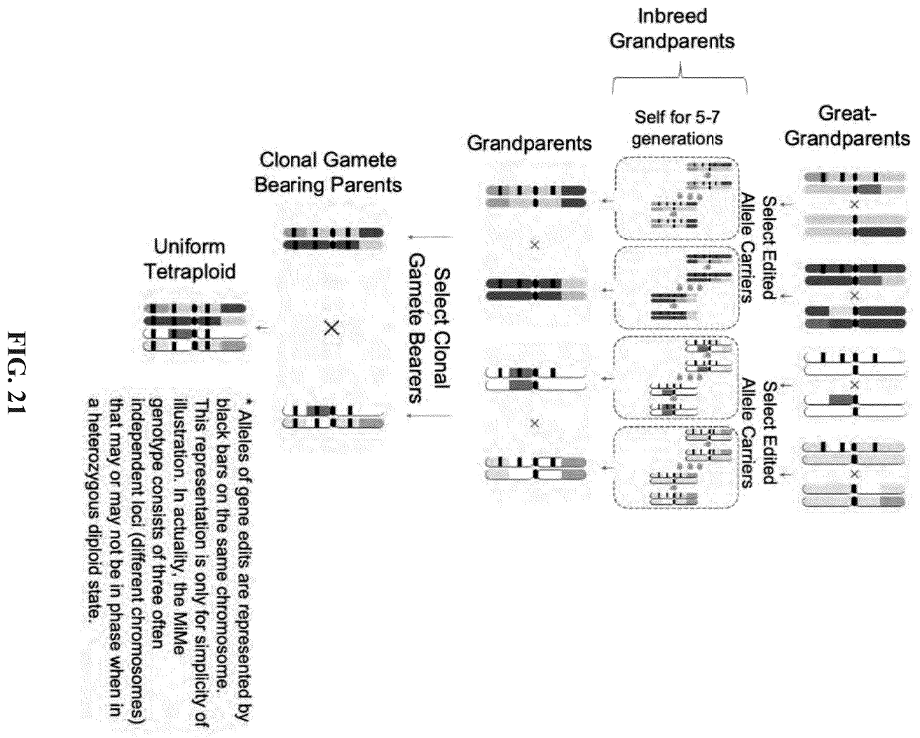

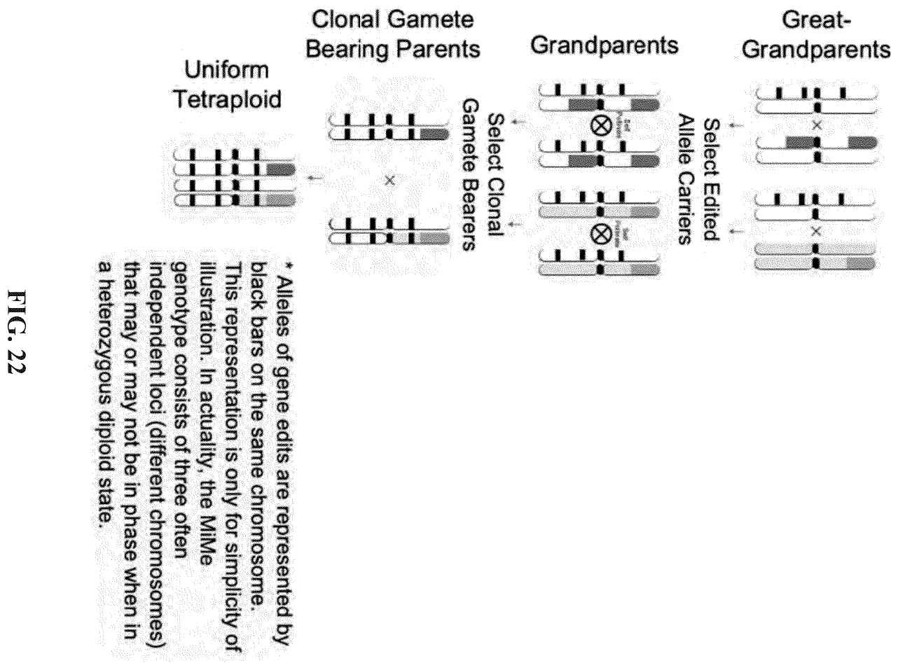

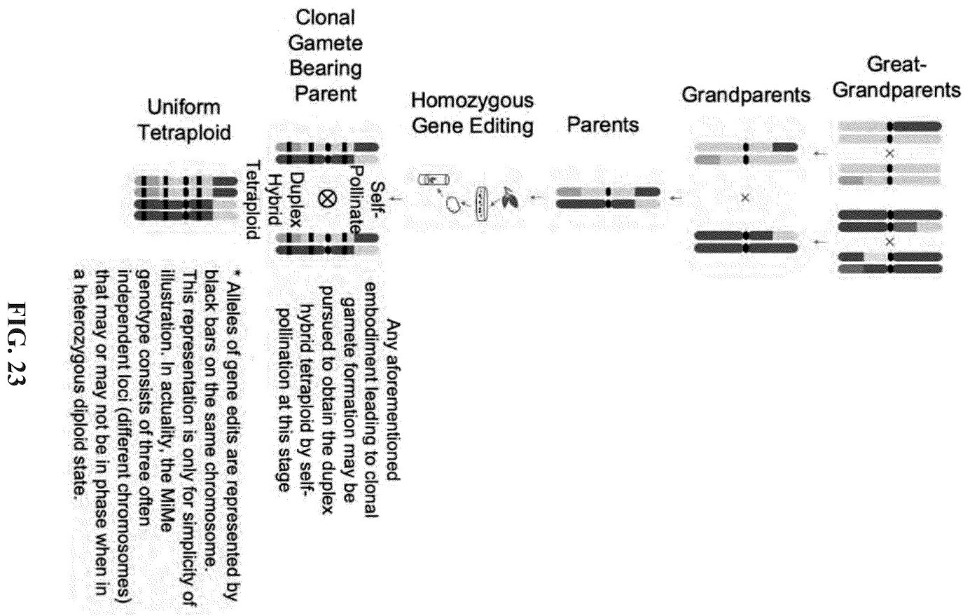

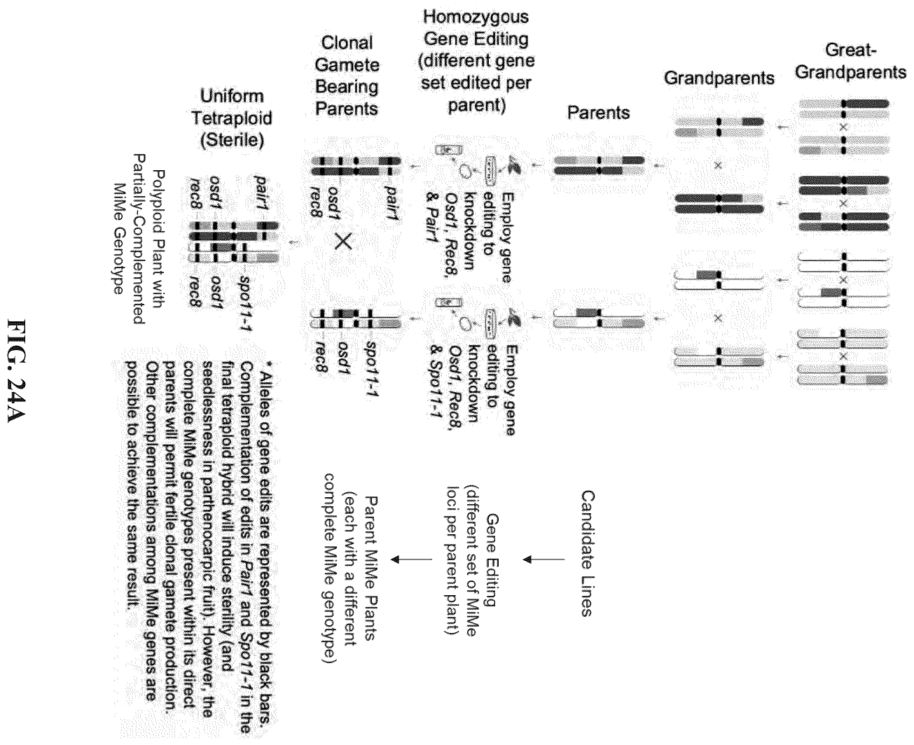

In certain embodiments, the generating of the one or more parent MiMe plants comprises introducing a partial MiMe genotype into two or more candidate lines to produce two or more grandparent non-MiMe plants each having a partial MiMe genotype and crossing said grandparent non-MiMe plants each having a partial MiMe genotype to produce the one or more parent MiMe plants, for example, as shown in FIG. 18 or 19. In some embodiments, the generating of the two parent MiMe plants comprises introducing a partial MiMe genotype into four candidate lines to produce four grandparent non-MiMe plants each having a partial MiMe genotype, and crossing pairs of said grandparent non-MiMe plants each having a partial MiMe genotype to produce the two parent MiMe plants, for example, as shown in FIG. 18 or 19. In other embodiments, the generating of the two parent MiMe plants comprises introducing a partial MiMe genotype into two candidate lines to produce two grandparent non-MiMe plants each having a partial MiMe genotype, crossing said grandparent non-MiMe plants each having a partial MiMe genotype to produce the first parent MiMe plant, and introducing a complete MiMe genotype directly into a third candidate line to produce the second parent MiMe plant. In additional embodiments, the generating of the one or more parent MiMe plants comprises introducing a partial MiMe genotype into a candidate line to produce a great-grandparent non-MiMe plant having a partial MiMe genotype, crossing the great-grandparent non-MiMe plant having a partial MiMe genotype with one or more candidate lines together comprising one or more haplotypes to produce a grandparent non-MiMe plant having a partial MiMe genotype and comprising two or more haplotypes, and crossing or selfing the grandparent non-MiMe plant having the partial MiMe genotype and comprising two or more haplotypes to produce a parent MiMe plant comprising two or more haplotypes, for example, as shown in any one of FIGS. 20-22. In one variation, the generating of the two parent MiMe plants comprises introducing a partial MiMe genotype into a candidate line to produce a great-grandparent non-MiMe plant having a partial MiMe genotype, crossing the great-grandparent non-MiMe plant having a partial MiMe genotype with two candidate lines together comprising two or more haplotypes to produce two grandparent non-MiMe plants together comprising three or more haplotypes, and selfing each of the grandparent non-MiMe plants together comprising three or more haplotypes to produce two parent MiMe plants together comprising three or more haplotypes, for example, as shown in FIG. 21 or 22. In one variation, the generating of the two parent MiMe plants comprises introducing a partial MiMe genotype into a candidate line to produce a great-grandparent non-MiMe plant having a partial MiMe genotype, crossing the great-grandparent non-MiMe plant having a partial MiMe genotype with two candidate lines together comprising two or more haplotypes to produce two grandparent non-MiMe plants together comprising three or more haplotypes, and crossing pairs of the grandparent non-MiMe plants together comprising three or more haplotypes to produce two parent MiMe plants together comprising three or more haplotypes, for example, as shown in FIG. 20. Any combination of methods for generating one or more parent MiMe plants described herein can be used in any combination to generate parent MiMe plants for use in the methods described herein. The grandparent non-MiMe plant or great-grandparent non-MiMe plant may have any partial MiMe genotype known in the art or described herein, including, but not limited to, partial MiMe genotypes comprising MiMe alleles resulting in decreased expression of any of the MiMe loci described herein.

In some embodiments, the grandparent non-MiMe plant has a partial MiMe genotype comprising (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, the grandparent non-MiMe plant has a partial MiMe genotype comprising (a) one or more MiMe alleles conferring decreased expression of one or more MiMe loci of each of a first and second MiMe component; and (a) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. The grandparent non-MiMe plant or great-grandparent non-MiMe plant may have any partial MiMe genotype known in the art or described herein, including, but not limited to, partial MiMe genotypes comprising MiMe alleles resulting in decreased expression of any of the MiMe loci described herein.

In some embodiments, the generating of the parent MiMe plants comprises introducing a complete MiMe genotype into two candidate lines wherein (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; (b) generating a second parent MiMe plant from one of the candidate lines, wherein the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component; (c) at least one of the MiMe loci having only MiMe alleles of the first MiMe component of the first parent MiMe plant is the same as at least one of the MiMe loci having only MiMe alleles of the first MiMe component of the second parent MiMe plant; and (d) either (1) at least one of the MiMe loci having only MiMe alleles of the third MiMe component of the first parent MiMe plant is the same as at least one of the MiMe loci having only MiMe alleles of the third MiMe component of the second parent MiMe plant, or (2) the one or more MiMe loci having only MiMe alleles of the third MiMe component of the first parent MiMe plant are distinct from the one or more MiMe loci having only MiMe alleles of the third MiMe component of the second parent MiMe plant. In some embodiments, the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. Exemplary MiMe loci of each of said MiMe components are extensively described herein.

In other embodiments, the generating of the parent MiMe plants comprises introducing a complete MiMe genotype into two candidate lines wherein (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component; and (c) at least one of the MiMe loci having only MiMe alleles of the first MiMe component of the first parent MiMe plant is the same as at least one of the MiMe loci having only MiMe alleles of the first MiMe component of the second parent MiMe plant. In some embodiments, the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. Exemplary MiMe loci of each of said MiMe components are extensively described herein.

In some embodiments, the grandparent non-MiMe plant or the great-grandparent non-MiMe plant has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the grandparent non-MiMe plant or the great-grandparent non-MiMe plant is a maize plant and the one or more OSD1 loci comprise OSD1-1, OSD1-2, and/or OSD1-3, and/or the one or more PAIR1 loci comprise PAIR1-1 and/or PAIR1-2. In other variations, the grandparent non-MiMe plant or the great-grandparent non-MiMe plant is a soybean plant and the one or more OSD1 loci comprise OSD1-1 and/or OSD1-2.

In some embodiments, the grandparent non-MiMe plant or the great-grandparent non-MiMe plant has a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In some variations, the grandparent non-MiMe plant or the great-grandparent non-MiMe plant is a maize plant and the one or more OSD1 loci comprise OSD1-1, OSD1-2, and/or OSD1-3. In other variations, the grandparent non-MiMe plant or the great-grandparent non-MiMe plant is a soybean plant and the one or more OSD1 loci comprise OSD1-1 and/or OSD1-2.

In some embodiments, the grandparent non-MiMe plant or the great-grandparent non-MiMe plant has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the grandparent non-MiMe plant or the great-grandparent non-MiMe plant has a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the grandparent non-MiMe plant or the great-grandparent non-MiMe plant is a maize plant and the one or more PAIR1 loci comprise PAIR1-1 and/or PAIR1-2.

In some embodiments, the grandparent non-MiMe plant or the great-grandparent non-MiMe plant has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In some variations, the grandparent non-MiMe plant or the great-grandparent non-MiMe plant is a soybean plant and the one or more TDM1 loci comprise TDM1-1 and/or TDM1-2.

In some embodiments, the grandparent non-MiMe plant or the great-grandparent non-MiMe plant has a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In some variations, the grandparent non-MiMe plant or the great-grandparent non-MiMe plant is a soybean plant and the one or more TDM1 loci comprise TDM1-1 and/or TDM1-2. In some variations, the grandparent non-MiMe plant or the great-grandparent non-MiMe plant is a maize plant and the one or more PAIR1 loci comprise PAIR1-1 and/or PAIR1-2.

In some embodiments, the grandparent non-MiMe plant or the great-grandparent non-MiMe plant has a partial MiMe genotype comprising (i) an os allele, wherein the grandparent non-MiMe plant or the great-grandparent non-MiMe plant is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the grandparent non-MiMe plant or the great-grandparent non-MiMe plant has a partial MiMe genotype comprising (i) a ps allele, wherein the grandparent non-MiMe plant or the great-grandparent non-MiMe plant is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1.

In some embodiments, the parent MiMe plant, the grandparent non-MiMe plant, the great-grandparent non-MiMe plant, or any combination thereof comprises one or more polynucleotide sequences selected from the group consisting of SEQ ID NOs: 464-496.

In some embodiments, the parent MiMe plant, the grandparent non-MiMe plant, the great-grandparent non-MiMe plant, or any combination thereof is a maize plant and comprises one or more polynucleotide sequences selected from the group consisting of SEQ ID NOs: 448-451. In certain embodiments, the parent MiMe plant, the grandparent non-MiMe plant, the great-grandparent non-MiMe plant, or any combination thereof comprises a) a MiMe allele at one or more OSD1-2 loci, each independently comprising a polynucleotide sequence selected from the group consisting of SEQ ID NOs: 448 and 449; b) a MiMe allele at a REC8 locus comprising the polynucleotide sequence of SEQ ID NO: 450; and/or c) a MiMe allele at a SPO11-1 locus comprising the polynucleotide sequence of SEQ ID NO: 451. In some variations, each of the one or more OSD1-2 loci, each of the one or more REC8 loci, each of the one or more PAM loci, and/or each of the one or more SPO11-1 loci are present on a different homologous chromosome.

In some embodiments, the parent MiMe plant, the grandparent non-MiMe plant, the great-grandparent non-MiMe plant, or any combination thereof is a potato plant and comprises one or more polynucleotide sequences selected from the group consisting of SEQ ID NOs: 464-496. In certain embodiments, parent MiMe plant, the grandparent non-MiMe plant, the great-grandparent non-MiMe plant, or any combination thereof comprises a MiMe allele at one or more SPO11-1 loci, each independently comprising a polynucleotide sequence selected from the group consisting of SEQ ID NOs: 464-466. In certain embodiments, parent MiMe plant, the grandparent non-MiMe plant, the great-grandparent non-MiMe plant, or any combination thereof comprises a) a MiMe allele at one or tore REC8 loci, each independently comprising a polynucleotide sequence selected from the group consisting of SEQ ID NOs: 468 and 469; and/or b) a MiMe allele; at one or more SPO11-1 loci, each independently comprising-K a polynucleotide sequence selected from the group consisting of SEQ ID NOs: 470 and 471. In certain embodiments, the parent MiMe plant, the grandparent non-MiMe plant, the great-grandparent non-MiMe plant, or any combination thereof comprises a) a MiMe allele are one or more REC8 loci, each independently comprising a polynucleotide sequence selected from the group consisting of SEQ ID NOs: 474 and 475; and/or b) a MiMe allele at one or more SPO11-1 loci, each independently comprising a polynucleotide sequence selected from the group consisting of SEQ ID NOs: 476 and 477. In certain embodiments, the parent MiMe plant, the grandparent non-MiMe plant, the great-grandparent non-MiMe plant, or any combination thereof comprises a MiMe allele are one or more SPO11-1 loci, each independently comprising a polynucleotide sequence selected from the group consisting of SEQ ID NOs: 478 and 479. In certain embodiments, the parent MiMe plant, the grandparent non-MiMe plant, the great-grandparent non-MiMe plant, or any combination thereof comprises a) a MiMe allele at one or more CYCA1 loci, each independently comprising a polynucleotide sequence selected from the group consisting of SEQ ID NOs: 480 and 481; b) a MiMe allele at one or more REC8 loci, each independently comprising a polynucleotide sequence selected from the group consisting of SEQ ID NOs: 482 and 483; and/or c) a MiMe allele at one or more SPO11-1 loci, each independently comprising a polynucleotide sequence selected from the group consisting of SEQ ID NOs: 484 and 485. In certain embodiments, the parent MiMe plant, the grandparent non-MiMe plant, the great-grandparent non-MiMe plant, or any combination thereof comprises a) a MiMe allele at one or more CYCA1 loci, each independently comprising a polynucleotide sequence selected from the group consisting of SEQ ID NOs: 486 and 487; b) a MiMe allele at one or more REC8 loci, each independently comprising a polynucleotide sequence selected from the group consisting of SEQ ID NOs: 488 and 489; and/or c) a MiMe allele at one or more SPO11-1 loci, each independently comprising a polynucleotide sequence selected from the group consisting of SEQ ID NOs: 490 and 491, In certain embodiments, the parent MiMe plant, the grandparent non-MiMe plant, the great-grandparent non-MiMe plant, or any combination thereof comprises a) a MiMe allele at one or mote REC8 loci, each independently comprising a polynucleotide sequence selected from the group consisting of SEQ ID NOs: 493 and 494; and/or b) a MiMe allele at one or more SPO11-1 loci, each independently comprising a polynucleotide sequence selected from the group consisting of SEQ ID NOs: 495 and 496. In some variations, each of the one or more OSD1 loci, each of the one or more REC8 loci, and/or each of the one or more SPO11-1 loci are present on a different homologous chromosome.

In some embodiments, the parent MiMe plant, the grandparent non-MiMe plant, the great-grandparent non-MiMe plant, or any combination thereof is an Arabidopsis plant and comprises one or more polynucleotide sequences selected from the group consisting of SEQ ID NOs: 452-463. In certain embodiments, the parent MiMe plant, the grandparent non-MiMe plant, the great-grandparent non-MiMe plant, or any combination thereof comprises a) a MiMe allele at one or more OSD1 loci, each independently comprising a polynucleotide sequence selected from the group consisting of SEQ ID NOs: 452 and 453; b) a MiMe allele at one or more REC8 loci, each independently comprising a polynucleotide sequence selected from the group consisting of SEQ ID NOs: 454 and 455; and/or c) a MiMe allele at one or more SPO11-1 loci, each independently comprising a polynucleotide sequence selected from the group consisting of SEQ ID NOs: 456 and 457 In certain embodiments, the parent MiMe plant, the grandparent non-MiMe plant, the great-grandparent non-MiMe plant, or any combination thereof comprises a) a MiMe allele at one or more OSD1 loci, each independently comprising a polynucleotide sequence selected from the group consisting of SEQ ID NOs: 458 and 459; b) MiMe allele at one or more REC loci, each independently comprising a polynucleotide sequence selected from the group consisting of SEQ ID NOs: 460 and 461; and/or c) 3 MiMe allele at one or more PAIR1 loci, each independently comprising a polynucleotide sequence selected from the group consisting of SEQ ID NOs: 462 and 463. In somite variations, each the one or more OSD1 loci, each of the one or more REC8 loci, each of the one or more PAIR1 loci, and/or each of the one or more SPO11-1 loci are present on differed homologous chromosome.

### Methods of Introducing Genetic Modifications

In some embodiments, the method of breeding a polyploid hybrid plant line comprising two, three, or more haplotypes comprises generating one or more parent MiMe plants from one or more of the two or more candidate lines, wherein the parent MiMe plant has a complete MiMe genotype, and wherein the complete MiMe genotype comprises alleles that are introduced via genetic modification. Generating a parent MiMe plant having a complete MiMe genotype may comprise introducing a complete MiMe genotype via genetic modification directly into a candidate line to produce the parent MiMe plant; introducing a partial MiMe genotype via genetic modification into two candidate lines to produce two grandparent non-MiMe plants each having a partial MiMe genotype, and crossing the grandparent non-MiMe plants each having a partial MiMe genotype to produce the parent MiMe plant; or a combination thereof. The MiMe alleles of a complete or partial MiMe genotype may be introduced via genetic modification by any means known in the art. In some embodiments, the genetic modifications are introduced by gene editing, transgenesis, or a combination thereof.

In some embodiments, the methods described herein comprise introducing one or more genetic modifications into one or more MiMe loci that result in the decreased expression of the one or more MiMe loci. In some embodiments, decreased expression a MiMe locus may be achieved by modifying or replacing nucleotide sequences of interest (such as a regulatory elements), gene disruption, gene knockout, gene knockdown, gene knock-in, gene silencing (including, e.g., by inserting and/or expressing an inverted repeat into a gene of interest), RNA interference (including, e.g., by insertion and/or expression of an RNA interference construct), expression of a repressor protein (e.g. dCas9), modification of methylation status of gene loci, modification of splicing sites, introducing alternate splicing sites, or any combination thereof. In some variations, the genetic modification is introduced into the first 70%, the first 60%, the first 50%, the first 40%, the first 30%, the first 20%, or the first 10% of the nucleotides of the coding sequence of the MiMe locus following the start codon in the 3′ direction. In certain variations, the genetic modification is introduced into the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction. In certain variations, the genetic modification is introduced into a non-coding element of the MiMe locus (e.g., a promoter, an enhancer, a terminator, an intron, or the like).

In some embodiments, the decreased expression of each of the one or more of the MiMe loci is independently achieved by introducing an insertion, a deletion, one or more nucleotide changes, or an inversion into the MiMe locus that that results in decreased expression of the MiMe locus. In some variations, the insertion, the deletion, the one or more nucleotide changes, or the inversion is introduced into the first 70%, the first 60%, the first 50%, the first 40%, the first 30%, the first 20%, or the first 10% of the nucleotides of the coding sequence of the MiMe locus following the start codon in the 3′ direction. In certain variations, the insertion, the deletion, the one or more nucleotide changes, or the inversion is introduced into the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction. In some variations, the insertion, the deletion, the one or more nucleotide changes, or the inversion eliminates expression (e.g., eliminates activity) of the MiMe locus. In some variations, the activity of the MiMe locus is eliminated by a premature stop codon introduced into the first 70%, the first 60%, the first 50%, the first 40%, the first 30%, the first 20%, or the first 10% of the nucleotides of the coding sequence of the MiMe locus following the start codon in the 3′ direction. In certain variations, the activity of the MiMe locus is eliminated by a premature stop codon introduced into the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the MiMe locus following the start codon in the 3′ direction.

In certain embodiments, the genetic modifications are introduced by gene editing. Any of several gene editing methods known in the art may be used to introduce the genetic modifications of the complete or partial MiMe genotype. In some variations, gene editing is performed with one or more natural or engineered nucleases including, but not limited to, RNA-guided nucleases, meganucleases, zinc finger nucleases (ZFNs), and transcription activator-like effector-based nucleases (TALENs). In further variations, gene editing is performed with RNA-guided nucleases including, but not limited to, Clustered Regularly Interspaced Short Palindromic Repeats (CRISPR) associated nucleases. Methods of gene editing are numerous, well-known and routine in the art, and are described in U.S. Ser. No. 17/045,747, U.S. Ser. No. 16/977,020, and U.S. Ser. No. 16/961,396, which are herein incorporated in their entirety.

An engineered nuclease may be a guided nuclease, which may function as a ribonucleoprotein (RNP) complex with a guide RNA. According to some embodiments, a guided nuclease may be selected from the group consisting of Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, CasZ, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions thereof). According to some embodiments, a guided nuclease is a Cas9 or Cpf1 enzyme. The DNA construct or molecule encoding a guided nuclease, or the guided nuclease itself, may be delivered with or without a guide nucleic acid.

For guided nucleases, a guide nucleic acid molecule may be further provided to direct the guided nuclease to a target site in the genome of the plant via base-pairing or hybridization to cause a DSB or nick at or near the target site. The guide nucleic acid may be transformed or introduced into a plant cell or tissue as a guide nucleic acid molecule, or as a recombinant DNA molecule, construct or vector comprising a transcribable DNA sequence encoding the guide nucleic acid operably linked to a promoter or plant-expressible promoter. The promoter may be a constitutive promoter, a tissue-specific or tissue-preferred promoter, a developmental stage promoter, or an inducible promoter.

In some embodiments, the guide nucleic acid comprises a first segment comprising a nucleotide sequence that is complementary to a sequence in a target nucleic acid and a second segment that interacts with a guided nuclease protein. In some embodiments, the first segment of a guide comprising a nucleotide sequence that is complementary to a sequence in a target nucleic acid corresponds to a CRISPR RNA (crRNA or crRNA repeat). In some embodiments, the second segment of a guide comprising a nucleic acid sequence that interacts with a guided nuclease protein corresponds to a trans-acting CRISPR RNA (tracrRNA). In some embodiments, the guide nucleic acid comprises two separate nucleic acid molecules (a polynucleotide that is complementary to a sequence in a target nucleic acid and a polynucleotide that interacts with a guided nuclease protein) that hybridize with one another. In other embodiments, the guide nucleic acid is a single polynucleotide. In some embodiments, the guide nucleic acid may comprise DNA, RNA or a combination of DNA and RNA.

A protospacer-adjacent motif (PAM) may be present in the genome immediately adjacent and upstream to the 5′ end of the genomic target site sequence complementary to the targeting sequence of the guide RNA, immediately downstream (3′) to the sense (+) strand of the genomic target site (relative to the targeting sequence of the guide RNA) as known in the art. See, e.g., Wu, X. et al. 2014. Target specificity of the CRISPR-Cas9 system, Quant Biol. 2(2): 59-70. The genomic PAM sequence on the sense (+) strand adjacent to the target site (relative to the targeting sequence of the guide RNA) may comprise any known PAM sequence, including, for example, 5′-NGG-3′. However, the corresponding sequence of the guide nucleic acid (immediately downstream (3′) to the targeting sequence of the guide RNA) may generally not be complementary to the genomic PAM sequence.

The guide nucleic acid may typically be a non-coding RNA molecule that does not encode a protein. The targeting sequence of the guide nucleic acid may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The targeting sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

In addition to the targeting sequence, a guide nucleic acid may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide nucleic acids for genome editing and site-directed integration at a target site within the genome of a plant using a guided nuclease are known in the art.

An engineered nuclease may be a site-specific nuclease. Several site-specific nucleases, such as recombinases, zinc finger nucleases (ZFNs), meganucleases, and TALENs, are not nucleic acid-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-nucleic acid-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such genomic locus to knockout or knockdown expression of the gene via repair of the DSB or nick, which may lead to the creation of a mutation or insertion of a sequence at the site of the DSB or nick, through cellular repair mechanisms, which may be guided by a donor template molecule.

In some embodiments, a site-specific nuclease is a recombinase. A recombinase may be a serine recombinase attached to a DNA recognition motif, a tyrosine recombinase attached to a DNA recognition motif, or other recombinase enzyme known in the art. A recombinase or transposase may be a DNA transposase or recombinase attached or fused to a DNA binding domain. Non-limiting examples of recombinases include a tyrosine recombinase attached, etc., to a DNA recognition motif provided herein is selected from the group consisting of a Cre recombinase, a gin recombinase, a Flp recombinase, and a Tnp1 recombinase. In an aspect, a Cre recombinase or a Gin recombinase provided herein is tethered to a zinc-finger DNA-binding domain, or a transcription activator-like effector (TALE) DNA-binding domain, or a Cas9 nuclease. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

A site-specific nuclease may be a zinc finger nuclease (ZFN). ZFNs are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger alpha-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., US Patent App. Nos. 2005/0064474, 2009/0117617, and 2012/0142062. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Without being limited by any theory, because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick.

A site-specific nuclease may be a TALEN enzyme. TALENs are artificial restriction enzymes generated by fusing the TALE DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, Sbfl, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Transcription activator-like effectors (TALEs) can be engineered to bind practically any DNA sequence, such as at or near the genomic locus of a gene in a plant. TALEs have a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al., Nucleic Acids Research (2012) 40: W117-122.; Cermak et al., Nucleic Acids Research (2011) 39:e82; and tale-nt.cac.cornelledu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SceI, I-Anil, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a gene. In another aspect, a meganuclease provided herein is capable of generating a targeted DSB.

In some embodiments, gene editing comprises (a) inducing a DSB in the genome of a cell at a cleavage site at or near a recognition site for a natural or engineered nuclease by expressing in the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site and/or a downstream flanking DNA region having homology to the DNA region downstream of the preselected site for allowing homologous recombination between said flanking region or regions and said DNA region or regions flanking said preselected site; and (c) selecting a cell wherein said repair nucleic acid molecule has been used as a template for making a modification of said genome at said preselected site. In other embodiments, gene editing comprises (a) inducing a DSB in the genome of a cell at a cleavage site at or near a recognition site for a natural or engineered nuclease by introducing into the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site and/or a downstream flanking DNA region having homology to the DNA region downstream of the preselected site for allowing homologous recombination between said flanking region or regions and said DNA region or regions flanking said preselected site; and (c) selecting a cell wherein said repair nucleic acid molecule has been used as a template for making a modification of said genome at said preselected site.

As used herein, a repair nucleic acid molecule is a single-stranded or double-stranded DNA molecule or RNA molecule that is used as a template for modification of the genomic DNA at the preselected site in the vicinity of or at the cleavage site. As used herein, use as a template for modification of the genomic DNA, means that the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two ends of the repair nucleic acid molecule (e.g. in case there is only one flanking region). Integration by homologous recombination will allow precise joining of the repair nucleic acid molecule to the target genome up to the nucleotide level, while NHEJ may result in small insertions/deletions at the junction between the repair nucleic acid molecule and genomic DNA.

In some embodiments, the genetic modifications introduced by gene editing result in the decreased expression (including non-expression or altered activity) of one or more MiMe loci. In gene editing, the introduction of a DSB or nick may be used to introduce targeted genetic modifications in the genome of a plant. According to this approach, genetic modifications, such as deletions, insertions, inversions and/or substitutions may be introduced at a target site via imperfect repair of the DSB or nick to produce a knock-out or knock-down of a gene, or to produce a MiMe component with altered activity. Such genetic modifications may be generated by imperfect repair of the targeted locus even without the use of a donor template molecule, and can result in decreased expression (including non-expression or altered activity) of an endogenous gene product. For example, genetic modifications may be produced by an indel (insertion or deletion of nucleotide bases in a target DNA sequence through NHEJ), or by specific removal of sequence that reduces or completely destroys the function of a sequence or motif at or near the targeting site, or which results in an altered activity of a MiMe component such as the production of a dominant-negative MiMe component, a constitutively active MiMe component, a null mutant, or the like. Such embodiments may comprise a deletion or insertion which alters one or more post-translational modifications on the one or more MiMe components. The post-translational modifications can include phosphorylation, glycosylation, ubiquitination, nitrosylation, methylation, acetylation, lipidation and the like. Altered activity in a MiMe component can be achieved, for example, by deleting or otherwise disrupting one or more phosphorylation sites (e.g., Tyrosine phosphorylation site or Serine/Threonine phosphorylation site). In further embodiments, the motif which is disrupted is a proteolytic cleavage site. A knockout of a gene may be achieved by inducing a DSB or nick at or near the endogenous locus of the gene that results in non-expression of the gene product, whereas a knockdown of a gene may be achieved in a similar manner by inducing a DSB or nick at or near the endogenous locus of the gene that is repaired imperfectly at a site that does not affect the coding sequence of the gene in a manner that would eliminate the function of the gene product. For example, the site of the DSB or nick within the endogenous locus may be in the upstream or 5′ region of the gene (e.g., a promoter and/or enhancer sequence) to affect or reduce its level of expression. Similarly, such targeted knockout or knockdown mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knockout mutations of a gene may be achieved by substituting, inserting, deleting or inverting at least a portion of the gene, including, but not limited to, by introducing a frame shift or premature stop codon into a protein coding sequence of the gene. A deletion of a portion of a gene may also be introduced by generating DSBs or nicks at two target sites and causing a deletion of the intervening target region flanked by the target sites.

In some embodiments, the genetic modifications are introduced by transgenesis. Transgenes may include, but are not limited to, one or more protein-coding sequences operably linked to a plant-expressible promoter, one or more transcribable DNA sequences encoding an RNA molecule operably linked to a plant-expressible promoter, a gene of interest, a marker gene, or any combination thereof. Methods for the introduction of transgenes in plants are well-known and routine in the art. In some embodiments, transgenesis comprises (a) inducing a DSB in the genome of a cell at a cleavage site at or near a recognition site for a natural or engineered nuclease by expressing in the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site, a downstream flanking DNA region having homology to the DNA region downstream of the preselected site, and a transgene region flanked by the upstream and downstream flanking DNA regions and comprising the transgene to be inserted at the preselected site; and (c) selecting a cell wherein said repair nucleic acid molecule has been used as a template for making a modification of said genome at said preselected site. In other embodiments, transgenesis comprises (a) inducing a DSB in the genome of a cell at a cleavage site at or near a recognition site for a natural or engineered nuclease by introducing into the cell the natural or engineered nuclease recognizing said recognition site and inducing said DSB at the cleavage site; (b) introducing into the cell a repair nucleic acid molecule comprising an upstream flanking region having homology to the DNA region upstream of the preselected site, a downstream flanking DNA region having homology to the DNA region downstream of the preselected site, and a transgene region flanked by the upstream and downstream flanking DNA regions and comprising the transgene to be inserted at the preselected site; and (c) selecting a cell wherein said repair nucleic acid molecule has been used as a template for making a modification of said genome at said preselected site.

In some embodiments, the genetic modification comprises introducing proteins, nucleic acids, or a combination thereof into a plant cell. The introduction of the proteins, nucleic acids, or combination thereof into the plant cell may be achieved by any of several means known and routinely-used in the art. In some embodiments, the introduction of the proteins, nucleic acids, or combination thereof into the plant cell comprises isolating protoplasts, transfecting the protoplasts, encapsulating the protoplasts, and regenerating plants from the protoplasts. In other embodiments, the introduction of the proteins, nucleic acids, or combination thereof into the plant cell comprises biolistic transformation. In certain embodiments, the introduction of the proteins, nucleic acids, or combination thereof into the plant cell comprises isolating immature plant embryos, bombarding the embryos with particles comprising nucleic acids, and regenerating plants from the immature embryos. Numerous additional transformation methods may be used to introduce the proteins, nucleic acids, or combination thereof into a suitable plant or plant cell. Transformation methods include the use of liposomes, electroporation, chemicals that increase free DNA uptake, injection of the DNA directly into the plant (cell) such as microinjection, particle gun bombardment, transformation using viruses or pollen and microprojection. Methods may be selected from the calcium/polyethylene glycol method for protoplasts (Krens et al. (1982) Nature 296: 72-74; Negrutiu et al. (1987) Plant. Mol. Biol. 8: 363-373); electroporation of protoplasts (Shillito et al. (1985) Bio/Technol. 3: 1099-1102); microinjection into plant material (Crossway et al. (1986) Mol. Gen. Genet. 202: 179-185); DNA or RNA-coated particle bombardment (Klein et al. (1987) Nature 327: 70) infection with (non-integrative) viruses and the like.

In some embodiments, generating the parent MiMe plant, the grandparent non-MiMe plant, the great-grandparent non-MiMe plant, or any combination thereof comprises introducing an RNA-guided nuclease system (e.g., a CRISPR system) into a cell of a candidate plant line, wherein the RNA-guided nuclease system (e.g., a CRISPR system) is configured to bind to one or more target sequences of each of one or more MiMe loci. Exemplary target sequences for specific MiMe loci are provided in the sequence listing as outlined in Table 9, and also in Examples 2, 5, and 7. In certain embodiments, the one or more candidate lines are potato plant lines, and the one or more target sequences comprise a polynucleotide sequence selected from the group consisting of SEQ ID NOs: 175-229, or a variant thereof comprising 1, 2, 3, 4, or 5 nucleotide substitutions. In certain embodiments, the one or more candidate lines are maize plant lines, and the one or more target sequences comprise a polynucleotide sequence selected from the group consisting of SEQ ID NOs: 230-276, or a variant thereof comprising 1, 2, 3, 4, or 5 nucleotide substitutions. In certain embodiments, the one or more candidate lines are Arabidopsis plant lines, and the one or more target sequences comprise a polynucleotide sequence selected from the group consisting of SEQ ID NOs: 386-447, or a variant thereof comprising 1, 2, 3, 4, or 5 nucleotide substitutions.

In certain embodiments, the CRISPR system comprises one or more nucleic acid molecules (e.g., one or more plasmids) encoding an expression cassette for expressing a Cas enzyme, and expression cassette for expressing a targeting RNA (e.g., a crRNA or gRNA molecule), or a combination thereof. In some variations, the targeting RNA and the Cas enzyme are encoded on separate nucleic acid molecules (e.g., separate plasmids). In other variations, the targeting RNA and the Cas enzyme are encoded on the same nucleic acid molecule (e.g., the same plasmid). In certain embodiments, the CRISPR system comprises ribonucleoproteins (RNPs) comprising a Cas enzyme complexed with a targeting RNA (e.g., a crRNA or gRNA molecule). Exemplary gRNA protospacer sequences for specific MiMe loci are provided in the sequence listing as outlined in Table 9, and also in Examples 2, 5, and 7. In certain embodiments, the one or more candidate lines are potato plant lines, and the targeting RNA (e.g., crRNA or gRNA) sequences comprises a polynucleotide sequence selected from the group consisting of SEQ ID NOs: 120-174, or a variant thereof comprising 1, 2, 3, 4, or 5 nucleotide substitutions. In certain embodiments, the one or more candidate lines are maize plant lines, and the targeting RNA (e.g., crRNA or gRNA) comprises a polynucleotide sequence selected from the group consisting of SEQ ID NOs: 277-323, or a variant thereof comprising 1, 2, 3, 4, or 5 nucleotide substitutions. In certain embodiments, the one or more candidate lines are Arabidopsis plant lines, and the targeting RNA (e.g., crRNA or gRNA) comprises a polynucleotide sequence selected from the group consisting of SEQ ID NOs: 234-385, or a variant thereof comprising 1, 2, 3, 4, or 5 nucleotide substitutions.

In some embodiments, introducing the CRISPR system into the cell of the candidate plant line results in an insertion, a deletion, one or more nucleotide changes, or an inversion that results in decreased expression of the targeted MiMe locus. The one or more MiMe loci may comprise any MiMe loci known in the art or described herein. In some variations, the insertion, the deletion, the one or more nucleotide changes, or the inversion is positioned in the first 70%, the first 60%, the first 50%, the first 40%, the first 30%, the first 20%, or the first 10% of the nucleotides of the coding sequence of the genomic locus following the start codon in the 3′ direction. In certain variations, the insertion, the deletion, the one or more nucleotide changes, or the inversion is positioned in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction. In some variations, the insertion, the deletion, the one or more nucleotide changes, or the inversion results in a premature stop codon present in the first 70%, the first 60%, the first 50%, the first 40%, the first 30%, the first 20%, or the first 10% of the nucleotides of the coding sequence of the MiMe locus following the start codon in the 3′ direction, thereby eliminating expression (e.g., activity) of the genomic locus. In some variations, the insertion, the deletion, the one or more nucleotide changes, or the inversion results in a premature stop codon present in the first 100, the first 200, the first 300, the first 400, the first 500, the first 600, the first 700, the first 800, the first 900, the first 1000, the first 1250, the first 1500, the first 1750, the first 2000, the first 2500, or the first 3000 nucleotides of the coding sequence of the genomic locus following the start codon in the 3′ direction, thereby eliminating expression (e.g., activity) of the genomic locus.

### Crossing Clonal Gametes to Produce Hybrid Polyploid Seed

In some embodiments, the method of breeding a polyploid hybrid plant line comprises providing clonal gametes from two parent MiMe plants and crossing the clonal gametes to produce a hybrid polyploid seed comprising two, three, or more haplotypes. In other embodiments, the method of breeding a polyploid hybrid plant line comprises providing clonal gametes from a parent MiMe plant, providing haploid gametes from a homozygous parent non-MiMe plant, and crossing the clonal gametes with the haploid gametes to produce a hybrid polyploid seed. In some variations, the homozygous parent non-MiMe plant is diploid and the haploid gametes are monoploid gametes. In yet other embodiments, the method of breeding a polyploid hybrid plant line comprises providing clonal gametes from a parent MiMe plant, providing unreduced, non-clonal gametes from a homozygous parent plant, and crossing the clonal gametes with the unreduced, non-clonal gametes to produce a hybrid polyploid seed. Methods for providing clonal gametes, providing haploid gametes, providing unreduced, non-clonal gametes, crossing clonal gametes, crossing clonal gametes with haploid gametes, and crossing clonal gametes with unreduced, non-clonal gametes are described herein. In some embodiments, the method of breeding a polyploid hybrid plant line comprises growing the hybrid polyploid seed to produce a hybrid polyploid plant.

In some embodiments, the method of breeding a polyploid hybrid plant line comprises crossing the clonal gametes to produce the hybrid polyploid seed. In some variations, crossing the clonal gametes to produce the hybrid polyploid seed comprises contacting pollen comprising clonal gametes with the stigma of a pistil comprising clonal gametes. In some variations, crossing the clonal gametes to produce the hybrid polyploid seed comprises contacting pollen comprising clonal gametes of a first plant with the stigma of a pistil comprising clonal gametes of a second plant. In some embodiments, crossing the clonal gametes to produce the hybrid polyploid seed comprises contacting pollen comprising clonal gametes from a first parent MiMe plant with the stigma of a pistil comprising clonal gametes of a second parent MiMe plant. In some variations, the homozygous parent non-MiMe plant is diploid and the haploid gametes are monoploid gametes. In some embodiments, the hybrid polyploid seed is produced by crossing the clonal gametes and allowing seeds to form.

In other embodiments, the method of breeding a polyploid hybrid plant line comprises crossing the clonal gametes with the haploid gametes to produce the hybrid polyploid seed. In one variation, crossing the clonal gametes with the haploid gametes to produce the hybrid polyploid seed comprises contacting pollen comprising clonal gametes with the stigma of a pistil comprising haploid gametes. In another variation, crossing the clonal gametes with the haploid gametes to produce the hybrid polyploid seed comprises contacting pollen comprising haploid gametes with the stigma of a pistil comprising clonal gametes. In some embodiments, crossing the clonal gametes with the haploid gametes to produce the hybrid polyploid seed comprises contacting pollen comprising clonal gametes from a parent MiMe plant with the stigma of a pistil comprising haploid gametes of a homozygous parent non-MiMe plant. In other embodiments, crossing the clonal gametes with the haploid gametes to produce the hybrid polyploid seed comprises contacting pollen comprising haploid gametes from a homozygous parent non-MiMe plant with the stigma of a pistil comprising clonal gametes of a parent MiMe plant. In some embodiments, the population of polyploid seed is produced by crossing the clonal gametes with the haploid gametes and allowing seeds to form.

In yet other embodiments, the method of breeding a polyploid hybrid plant line comprises crossing clonal gametes with unreduced, non-clonal gametes to produce the hybrid polyploid seed. In one variation, crossing the clonal gametes with the unreduced, non-clonal gametes to produce the hybrid polyploid seed comprises contacting pollen comprising clonal gametes with the stigma of a pistil comprising unreduced, non-clonal gametes. In another variation, crossing the clonal gametes with the unreduced, non-clonal gametes to produce the hybrid polyploid seed comprises contacting pollen comprising unreduced, non-clonal gametes with the stigma of a pistil comprising clonal gametes. In some embodiments, crossing the clonal gametes with the unreduced, non-clonal gametes to produce the hybrid polyploid seed comprises contacting pollen comprising clonal gametes from a parent MiMe plant with the stigma of a pistil comprising unreduced, non-clonal gametes of a homozygous parent plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, crossing the clonal gametes with the unreduced, non-clonal gametes to produce the hybrid polyploid seed comprises contacting the stigma of a pistil comprising clonal gametes from a parent MiMe plant with pollen comprising unreduced, non-clonal gametes of a homozygous parent plant that is homozygous for (a) a MiMe allele conferring decreased expression of one or more MiMe loci of a component of progression through the first division of meiosis, or (b) a MiMe allele conferring decreased expression of one or more MiMe loci of a component of progression through the second division of meiosis. In some embodiments, the hybrid polyploid seed is produced by crossing the clonal gametes with the unreduced, non-clonal gametes and allowing seeds to form.

### Evaluating Characteristics and Heterotic Performance

In some embodiments, the method of breeding a polyploid hybrid plant line comprises evaluating one or more characteristics of the hybrid polyploid plant. Methods for evaluating plant characteristics are numerous and well-known in the art. The one or more characteristics evaluated may include, but are not limited to, plant height, plant size, plant vigor, fruit yield, crop yield, disease resistance, pest resistance, and the like. The plants to be evaluated may then be grown under different geographical, climatic, and soil conditions, and further selections can be made during, and at the end of, the growing season. Promising advanced breeding lines are thoroughly tested and compared to appropriate standards in environments representative of the commercial target area(s) for three years at least. The best lines are candidates for new commercial cultivars. These processes, which lead to the final step of marketing and distribution, usually take from five to ten years from the time the first cross or selection is made.

In some embodiments, the one or more characteristics includes the heterotic performance of the two, three, or more haplotypes of the polyploid hybrid plant. In some embodiments, the heterotic performance of the two, three, or more haplotypes of the polyploid hybrid plant is used to guide the breeding of lines, the selecting of candidate lines, or both. In some embodiments, the heterotic performance is predicted via genome prediction modeling.

In some embodiments, the method of breeding a polyploid hybrid plant line comprises repeating the steps of the method using the one or more characteristics of the hybrid polyploid plant evaluated to guide the breeding of, the selecting of candidate lines, or both. In some variations, the method comprises repeating the steps of the method two, three, four, five, six, seven, eight, nine, or ten times or more. In additional variations, the repeating of the steps of the method iteratively informs a genome prediction model for improved prediction of heterotic performance. In certain variations, the improved prediction of heterotic performance allows for rapid combination of haplotypes with strong heterotic performance and acceleration of breeding programs.

### Inventory and Maintenance of Lines

In some embodiments, the method of breeding a hybrid polyploid plant line comprises maintaining the lines of the plant. In some variations, the lines of the plant are maintained via vegetative propagation, selfing, apomixis, cell culture, or any combination thereof. Additional methods of maintaining lines of plants are well-known in the art. In some embodiments, the method of breeding a hybrid polyploid plant line comprises maintaining an inventory of lines of the plant from which haplotypes may be selected for rapid deterministic stacking of the haplotypes. In some variations, the inventory of lines comprises one or more plant lines having complete or partial MiMe genotypes that are maintained through vegetative propagation, hybridization with a haploid inducer, or a combination thereof. In additional variations, the inventory of lines may include, but is not limited to, the set of lines, candidate lines, lines comprising one or more MiMe alleles, lines having a partial MiMe genotype, lines having a complete MiMe genotype, grandparent non-MiMe plants having a partial MiMe genotype, parent MiMe plants, homozygous parent non-MiMe plants, or any combination thereof maintained by vegetative propagation, seed propagation, tissue culture, hybridization with a haploid inducer or any combination thereof. In some embodiments, maintaining the inventory of lines of the plant comprises maintaining pairs of parent MiMe plants having any of the complete MiMe genotypes or complementary genotypes described herein.

In some embodiments, haploid induction may be used to maintain polyploid plant lines having a complete MiMe genotype. In certain embodiments, maintaining the inventory of lines of the plant comprises crossing a polyploid plant having a complete MiMe genotype with a haploid inducer to produce progeny of the same ploidy as the plant having the complete MiMe genotype. In one variation, maintaining the inventory of lines of the plant comprises crossing a tetraploid plant having a complete MiMe genotype with a haploid inducer to produce tetraploid progeny.

### Genetically Modified Plants, Plant Parts, Plant Cells, and Processed Plant Products

In yet another aspect, provided herein are genetically modified plants, plant parts, and plant cells grown from a population of polyploid seed or a subpopulation of genetically uniform polyploid seed described herein. Also provided herein are processed plant products derived from the genetically modified plants, plant parts, or plant cells provided herein. In some embodiments, the genetically modified plant parts, genetically modified plant cells, and processed plant products provided herein are non-regenerable.

In some embodiments, genetically modified plants and genetically modified plant parts are provided herein. In certain embodiments, the genetically modified plants and plant parts may be grown from a population of polyploid seed or a subpopulation of genetically uniform polyploid seed comprising three or more haplotypes and one or more genetic modifications resulting in decreased expression of one or more MiMe loci described herein. In other embodiments, the genetically modified plants and plant parts may be regenerated from a genetically modified plant cell wherein the genetically modified plant cell comprises three or more haplotypes and one or more genetic modifications resulting in decreased expression of one or more MiMe loci. In yet other embodiments, the genetically modified plants and plant parts may be grown from a population of polyploid seed or a subpopulation of genetically uniform polyploid seed having a partially-complemented MiMe genotype described herein. In still other embodiments, the genetically modified plants and plant parts may be regenerated from a genetically modified plant cell wherein the genetically modified plant cell has a partially-complemented MiMe genotype described herein. Genetically modified plants can be obtained from a genetically modified seed. Genetically modified plant parts can be obtained by cutting, snapping, grinding or otherwise disassociating the part from the plant. The plant part may be any plant part known in the art, including, but not limited to, a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, a seed, a boll, a stamen, a pericarp, an endosperm, a cob, a glume, a husk, a leaf sheath, a ligule, a trichome, or any portion thereof. In some embodiments, the plant part is a seedless fruit. In certain embodiments, a genetically modified plant part provided herein is a non-regenerable portion of a genetically modified plant part. As used in this context, a “non-regenerable” portion of a genetically modified plant part refers to a portion that cannot be induced to form a whole plant or that cannot be induced to form a whole plant (e.g., through in vitro culture) that is capable of sexual and/or asexual reproduction. A non-regenerable portion of a genetically modified plant part may be a portion of any plant part known in the art, including, but not limited to, a flower, a pistil, a leaf, a stem, a petiole, a cutting, a tissue, a seed coat, an ovule, pollen, a root, a rootstock, a scion, a fruit (e.g., a seedless fruit), a cotyledon, a hypocotyl, a protoplast, an embryo, an anther, a boll, or any portion thereof.

In some embodiments, a non-regenerable or non-propagating plant cell is provided herein. As used in this context, a “non-regenerable plant cell” is a cell which cannot be regenerated into a whole plant that is capable of sexual and/or asexual reproduction through in vitro culture. The non-regenerable cell may be in a plant or plant part described herein. The non-regenerable cell may be a cell in a seed, or in the seedcoat of said seed. Alternatively, the non-regenerable cell may be a cell in a seedless fruit. Mature plant organs, including a mature leaf, a mature stem, a mature root, or a mature fruit contain at least one non-regenerable cell. In certain embodiments, the non-regenerable plant cell is a somatic cell.

Also provided herein is a cell culture or tissue culture of non-regenerable or regenerable cells or tissue of a genetically modified plant or genetically modified plant part described herein, wherein the non-regenerable or regenerable cells comprise three or more haplotypes and one or more genetic modifications resulting in decreased expression of one or more MiMe loci described herein. Also provided herein is a cell culture or tissue culture of non-regenerable or regenerable cells or tissue of a genetically modified plant or genetically modified plant part described herein, wherein the non-regenerable or regenerable cells have a partially-complemented MiMe genotype described herein. Preferably, the regenerable cells are derived from embryos, protoplasts, meristematic cells, callus, pollen, leaves, anthers, stems, petioles, roots, root tips, fruits, seeds, flowers, cotyledons, and/or hypocotyls of a genetically modified plant or a genetically modified plant part grown from a population of polyploid seed or a subpopulation of genetically uniform polyploid seed described herein.

In some embodiments, provided herein is a processed plant product derived from a genetically modified plant, plant part, or plant cell described herein comprising three or more haplotypes and one or more genetic modifications resulting in decreased expression of one or more MiMe loci. In certain embodiments, the processed plant product contains sufficient nucleic acid (e.g., DNA or RNA) and/or protein material from the genetically modified plant, plant part, or plant cell to detect nucleic acid and/or protein sequences corresponding to the three haplotypes, the one or more genetic modifications resulting in decreased expression of one or more MiMe loci, or both. In some embodiments, provided herein is a processed plant product derived from a genetically modified plant, plant part, or plant cell described herein having a partially-complemented MiMe genotype described herein. In certain embodiments, the processed plant product contains sufficient nucleic acid (e.g., DNA or RNA) and/or protein material from the genetically modified plant, plant part, or plant cell to detect nucleic acid and/or protein sequences corresponding to the partially-complemented MiMe genotype. In some embodiments, the processed plant product is non-regenerable, i.e., cannot be induced to form a whole plant or that cannot be induced to form a whole plant that is capable of sexual and/or asexual reproduction.

A processed plant product may be a seed, a fruit (e.g., a seedless fruit), a grain, a root, a vegetable, or any plant part described herein, and may be blended as a commodity or other product which moves through commerce and is derived from a genetically modified plant or a genetically modified plant part. In some embodiments, the commodity or other product can be tracked through commerce by detecting nucleic acid and/or protein sequences of the genetically modified plant or plant part from which they were obtained. In certain embodiments, the processed plant product comprises a detectable amount of nucleotide and/or protein sequences corresponding to the three or more haplotypes and/or to the one or more genetic modifications resulting in decreased expression of one or more MiMe loci. In certain embodiments, the processed plant product comprises a detectable amount of nucleotide and/or protein sequences corresponding to the partially-complemented MiMe genotype and, optionally, to the two, three, or more haplotypes. In certain embodiments, the commodity or other product is produced in or maintained in the genetically modified plant or plant part from which the commodity or other product has been obtained. Such commodities or other products of commerce include, but are not limited to, plant parts, biomass, oil, meal, food starch, syrup, sugar, animal feed, flour, flakes, bran, lint, hull, processed seed, seed, seedless fruit, puree, juice, juice concentrate, pulp, pomace, preserve, or sauce. The processed plant product may be a food product that is processed by any means known in the art, e.g., canned, steamed, boiled, fried, blanched, juiced, pureed, and/or frozen etc.

In yet another aspect, provided herein are genetically modified maize plants, plant parts, and plant cells grown from a population of polyploid maize seed or a subpopulation of genetically uniform polyploid maize seed described herein. Also provided herein are processed maize products derived from the genetically modified maize plants, plant parts, or plant cells provided herein. In some embodiments, the genetically modified maize plant parts, genetically modified plant cells, and processed maize products provided herein are non-regenerable.

In some embodiments, genetically modified maize plants and genetically modified plant parts are provided herein. The genetically modified maize plants and plant parts may be grown from a population of polyploid maize seed or a subpopulation of genetically uniform polyploid maize seed comprising three or more haplotypes and one or more genetic modifications resulting in decreased expression of one or more MiMe loci described herein. Alternatively, the genetically modified maize plants and plant parts may be regenerated from a genetically modified maize plant cell wherein the genetically modified maize plant cell comprises three or more haplotypes and one or more genetic modifications resulting in decreased expression of one or more MiMe loci. Genetically modified maize plants can be obtained from a genetically modified maize seed. Genetically modified maize plant parts can be obtained by cutting, snapping, grinding or otherwise disassociating the part from the maize plant. The maize plant part may be any plant part known in the art, including, but not limited to, a flower, a pistil, a stamen, a leaf, a stem, a cutting, a tissue, a seed coat, an ovule, pollen, a root, a rootstock, a scion, a pericarp, a cotyledon, a hypocotyl, a protoplast, an embryo, an endosperm, an anther, a seed, a cob, a glume, a husk, a leaf sheath, a ligule, a trichome, or a portion thereof. In certain embodiments, a genetically modified maize plant part provided herein is a non-regenerable portion of a genetically modified maize plant part. As used in this context, a “non-regenerable” portion of a genetically modified maize plant part refers to a portion that cannot be induced to form a whole maize plant (e.g., through in vitro culture) or that cannot be induced to form a whole maize plant that is capable of sexual and/or asexual reproduction. A non-regenerable portion of a genetically modified maize plant part may be a portion of a flower, a pistil, a stamen, a leaf, a stem, a cutting, a tissue, a seed coat, an ovule, pollen, a root, a rootstock, a scion, a pericarp, a cotyledon, a hypocotyl, a protoplast, an embryo, an endosperm, an anther, a cob, a glume, a husk, a leaf sheath, a ligule, a trichome, or a portion thereof.

In some embodiments, a non-regenerable or non-propagating maize plant cell is provided herein. As used in this context, a “non-regenerable plant cell” is a cell which cannot be regenerated into a whole maize plant that is capable of sexual and/or asexual reproduction through in vitro culture. The non-regenerable maize cell may be in a maize plant or plant part described herein. The non-regenerable maize cell may be a cell in a seed, or in the seedcoat of said seed. Mature maize plant organs, including a mature leaf, a mature stem or a mature root, contain at least one non-regenerable cell. In certain embodiments, the non-regenerable maize plant cell is a somatic cell.

Also provided herein is a maize cell culture or tissue culture of non-regenerable or regenerable maize cells or tissue of a genetically modified maize plant or genetically modified plant part described herein, wherein the non-regenerable or regenerable maize cells comprise three or more haplotypes and one or more genetic modifications resulting in decreased expression of one or more MiMe loci described herein. Preferably, the regenerable maize cells are derived from embryos, protoplasts, meristematic cells, callus, pollen, leaves, anthers, stems, pericarps, roots, root tips, seeds, flowers, cotyledons, and/or hypocotyls of a genetically modified maize plant or a genetically modified plant part grown from a population of polyploid maize seed or a subpopulation of genetically uniform polyploid maize seed described herein.

In some embodiments, provided herein is a processed maize product derived from a genetically modified maize plant, plant part, or plant cell described herein comprising three or more haplotypes and one or more genetic modifications resulting in decreased expression of one or more MiMe loci. In certain embodiments, the processed maize product contains sufficient nucleic acid (e.g., DNA or RNA) and/or protein material from the genetically modified maize plant, plant part, or plant cell to detect nucleic acid and/or protein sequences corresponding to the three haplotypes, the one or more genetic modifications resulting in decreased expression of one or more MiMe loci, or both. In some embodiments, the processed maize product is non-regenerable, i.e., cannot be induced to form a whole maize plant or that cannot be induced to form a whole maize plant that is capable of sexual and/or asexual reproduction.

A processed maize product may be a seed, a grain, a root, a vegetable, or any maize plant part described herein, and may be blended as a commodity or other product which moves through commerce and is derived from a genetically modified maize plant or a genetically modified plant part. In some embodiments, the commodity or other product can be tracked through commerce by detecting nucleic acid and/or protein sequences of the genetically modified maize plant or plant part from which they were obtained. In certain embodiments, the processed maize product comprises a detectable amount of nucleotide and/or protein sequences corresponding to the three or more haplotypes and/or to the one or more genetic modifications resulting in decreased expression of one or more MiMe loci. In certain embodiments, the commodity or other maize product is produced in or maintained in the genetically modified maize plant or plant part from which the commodity or other product has been obtained. Such commodities or other products of commerce include, but are not limited to, maize plant parts, fresh corn, canned corn, dehydrated corn, starch, hulls, hominy, popcorn, cereal, grain, margarine, fermented alcoholic beverage, biofuel, gluten, corn syrup, table syrup, candy, confections, soft drinks, ice cream, shoe polish, corn sugar, infant formulas, dietetic foods, caramel coloring, vinegar, lactic acid, tanning mixtures, brewing additive, artificial silk, edible starch, dextrin, mucilage, glue, textile sizing, food sauces, fireworks, industrial starch, laundry starch, filler in paper, cosmetics, explosives, germ, oil cake or meal, cattle feed, plastic resin, rubber substitutes, erasers, elastic heels, soap, glycerin, soluble corn oil, cloth coloring, salad oils, cooking oils, medicinal oils, animal feed, paper, wallboard, filling material, fuel, charcoal, industrial solvent, biomass, oil, meal, food starch, syrup, sugar, animal feed, flour, flakes, bran, processed seed, and seed. The processed maize product may be a food product that is processed by any means known in the art, e.g., canned, steamed, boiled, fried, blanched and/or frozen etc. The maize product may be produced for any purpose(s) or industry, including but not limited to human consumption, agriculture, animal consumption, dietary supplement, food product ingredient, pharmaceutical, textile, wood, paper, adhesive, binder, texture agent, filler, biofuel production.

In yet another aspect, provided herein are genetically modified potato plants, plant parts, and plant cells grown from a population of polyploid potato seed or a subpopulation of genetically uniform polyploid potato seed described herein. Also provided herein are processed potato products derived from the genetically modified potato plants, plant parts, or plant cells provided herein. In some embodiments, the genetically modified potato plant parts, genetically modified plant cells, and processed potato products provided herein are non-regenerable.

In some embodiments, genetically modified potato plants and genetically modified plant parts are provided herein. The genetically modified potato plants and plant parts may be grown from a population of polyploid potato seed or a subpopulation of genetically uniform polyploid potato seed comprising three or more haplotypes and one or more genetic modifications resulting in decreased expression of one or more MiMe loci described herein. Alternatively, the genetically modified potato plants and plant parts may be regenerated from a genetically modified potato plant cell wherein the genetically modified potato plant cell comprises three or more haplotypes and one or more genetic modifications resulting in decreased expression of one or more MiMe loci. Genetically modified potato plants can be obtained from a genetically modified potato seed. Genetically modified potato plant parts can be obtained by cutting, snapping, grinding or otherwise disassociating the part from the potato plant. The potato plant part may be any plant part known in the art, including, but not limited to, a flower, a pistil, a leaf, a stem, a petiole, a cutting, a tissue, a seed coat, an ovule, a microspore, pollen, a tuber, a stolon, a meristem, a root, a rootstock, a scion, a fruit, a cotyledon, a hypocotyl, a protoplast, an embryo, an anther, a seed, or any portion thereof. In certain embodiments, a genetically modified potato plant part provided herein is a non-regenerable portion of a genetically modified potato plant part. As used in this context, a “non-regenerable” portion of a genetically modified potato plant part refers to a portion that cannot be induced to form a whole potato plant or that cannot be induced to form a whole potato plant (e.g., through in vitro culture) that is capable of sexual and/or asexual reproduction. A non-regenerable portion of a genetically modified potato plant part may be a portion of a flower, a pistil, a leaf, a stem, a petiole, a cutting, a tissue, a seed coat, an ovule, a microspore, pollen, a tuber, a stolon, a root, a rootstock, a scion, a fruit, a cotyledon, a hypocotyl, a protoplast, an embryo, an anther, or any portion thereof.

In some embodiments, a non-regenerable or non-propagating potato plant cell is provided herein. As used in this context, a “non-regenerable plant cell” is a cell which cannot be regenerated into a whole potato plant that is capable of sexual and/or asexual reproduction through in vitro culture. The non-regenerable potato cell may be in a potato plant or plant part described herein. The non-regenerable potato cell may be a cell in a seed, or in the seedcoat of said seed. Mature potato plant organs, including a mature leaf, a mature stem or a mature root, contain at least one non-regenerable cell. In certain embodiments, the non-regenerable potato plant cell is a somatic cell.

Also provided herein is a potato cell culture or tissue culture of non-regenerable or regenerable potato cells or tissue of a genetically modified potato plant or genetically modified plant part described herein, wherein the non-regenerable or regenerable potato cells comprise three or more haplotypes and one or more genetic modifications resulting in decreased expression of one or more MiMe loci described herein. Preferably, the regenerable potato cells are derived from embryos, protoplasts, meristematic cells, callus, microspores, pollen, leaves, tubers, stolons, anthers, stems, petioles, roots, root tips, fruits, seeds, flowers, cotyledons, and/or hypocotyls of a genetically modified potato plant or a genetically modified plant part grown from a population of polyploid potato seed or a subpopulation of genetically uniform polyploid potato seed described herein.

In some embodiments, provided herein is a processed potato product derived from a genetically modified potato plant, plant part, or plant cell described herein comprising three or more haplotypes and one or more genetic modifications resulting in decreased expression of one or more MiMe loci. In certain embodiments, the processed potato product contains sufficient nucleic acid (e.g., DNA or RNA) and/or protein material from the genetically modified potato plant, plant part, or plant cell to detect nucleic acid and/or protein sequences corresponding to the three haplotypes, the one or more genetic modifications resulting in decreased expression of one or more MiMe loci, or both. In some embodiments, the processed potato product is non-regenerable, i.e., cannot be induced to form a whole potato plant or that cannot be induced to form a whole potato plant that is capable of sexual and/or asexual reproduction.

A processed potato product may be a seed, a tuber, a fruit, a grain, a root, a stolon, a vegetable, or any potato plant part described herein, and may be blended as a commodity or other product which moves through commerce and is derived from a genetically modified potato plant or a genetically modified plant part. In some embodiments, the commodity or other product can be tracked through commerce by detecting nucleic acid and/or protein sequences of the genetically modified potato plant or plant part from which they were obtained. In certain embodiments, the processed potato product comprises a detectable amount of nucleotide and/or protein sequences corresponding to the three or more haplotypes and/or to the one or more genetic modifications resulting in decreased expression of one or more MiMe loci. In certain embodiments, the commodity or other potato product is produced in or maintained in the genetically modified potato plant or plant part from which the commodity or other product has been obtained. Such commodities or other products of commerce include, but are not limited to, potato plant parts, biomass, oil, meal, food starch, syrup, sugar, animal feed, flour, flakes, processed seed, seed, potato fries (French fries), wedges, shredded potato products (e.g., hash browns, tater tots), baked potatoes, fresh potatoes, mashed potatoes, dehydrated potatoes, pellets, abraded peels, steamed peels, potato slurry, puree, filter cake, gray starch, screen solids, pulp, potato protein concentrate, culled fries, culled crisps, crowns, batter, crumbles, nubbins, or material fermented for alcoholic beverage production. The processed potato product may be a food product that is processed by any means known in the art, e.g., canned, steamed, boiled, fried, blanched and/or frozen etc. The potato product may be produced for any purpose or industry, including but not limited to human consumption, animal consumption, dietary supplement, food product ingredient, pharmaceutical, textile, wood, paper, adhesive, binder, texture agent, filler, washing of boreholes or biofuel production.

In some embodiments, provided herein is a genetically modified plant, plant part, or plant cell comprising: i) three or more haplotypes; and ii) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, provided herein is a genetically modified plant, plant part, or plant cell comprising: i) three or more haplotypes; and ii) a partial MiMe genotype comprising: (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof. In certain embodiments, the genetically modified plant part is a non-regenerable plant part. In certain embodiments, the genetically modified plant cell is a non-regenerable plant cell. In some embodiments, the genetically modified plant, plant part, or plant cell is a maize plant, a maize plant part, or a maize plant cell. In some embodiments, the genetically modified plant, plant part, or plant cell is a potato plant, a potato plant part, or a potato plant cell. Also provided herein is germplasm of the genetically modified plant, the germplasm comprising the complete or partial MiMe genotype. Further provided herein is a genetically altered plant genome derived from the genetically modified plant, the genetically altered plant genome comprising the complete or partial MiMe genotype.

In some embodiments, provided herein is a genetically modified plant, plant part, or plant cell comprising: i) three or more haplotypes; and ii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, provided herein is a genetically modified plant, plant part, or plant cell comprising: i) three or more haplotypes; and ii) a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In other variations, the one or more MiMe loci of the component of progression through the first division of meiosis comprise PS1, JASON, or a combination thereof. In certain embodiments, the genetically modified plant part is a non-regenerable plant part. In certain embodiments, the genetically modified plant cell is a non-regenerable plant cell. In some embodiments, the genetically modified plant, plant part, or plant cell is a maize plant, a maize plant part, or a maize plant cell. In some embodiments, the genetically modified plant, plant part, or plant cell is a potato plant, a potato plant part, or a potato plant cell. Also provided herein is germplasm of the genetically modified plant, the germplasm comprising the complete or partial MiMe genotype. Further provided herein is a genetically altered plant genome derived from the genetically modified plant, the genetically altered plant genome comprising the complete or partial MiMe genotype.

In some embodiments, provided herein is a genetically modified plant, plant part, or plant cell comprising: (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the MiMe locus of the component of progression through the first division of meiosis of the third haplotype is PS1 or JASON. In still additional variations, the one or more MiMe loci of the component of progression through the second division of meiosis of the first and second haplotype comprise OSD1, CYCA1, TDM1, PC1, PC2, FC, or any combination thereof. In yet additional variations, the locus of the component of progression through the second division of meiosis of the third haplotype is OSD1, CYCA1, TDM1, PC1, PC2, or FC. In certain embodiments, the genetically modified plant part is a non-regenerable plant part. In certain embodiments, the genetically modified plant cell is a non-regenerable plant cell. In some embodiments, the genetically modified plant, plant part, or plant cell is a maize plant, a maize plant part, or a maize plant cell. In some embodiments, the genetically modified plant, plant part, or plant cell is a potato plant, a potato plant part, or a potato plant cell. Also provided herein is germplasm of the genetically modified plant, the germplasm comprising the MiMe alleles of the genetically modified plant. Further provided herein is a genetically altered plant genome derived from the genetically modified plant, the genetically altered plant genome comprising the germplasm comprising the MiMe alleles of the genetically modified plant.

In some embodiments, provided herein is a genetically modified plant, plant part, or plant cell comprising: (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis of the first and second haplotype comprise PS1, JASON, or a combination thereof. In yet additional variations, the MiMe locus of the component of progression through the first division of meiosis of the third haplotype is PS1 or JASON. In still additional variations, the MiMe locus of the component of progression through the second division of meiosis of the third haplotype is OSD1, CYCA1, TDM1, PC1, PC2 or FC. In certain embodiments, the genetically modified plant part is a non-regenerable plant part. In certain embodiments, the genetically modified plant cell is a non-regenerable plant cell. In some embodiments, the genetically modified plant, plant part, or plant cell is a maize plant, a maize plant part, or a maize plant cell. In some embodiments, the genetically modified plant, plant part, or plant cell is a potato plant, a potato plant part, or a potato plant cell. Also provided herein is germplasm of the genetically modified plant, the germplasm comprising the MiMe alleles of the genetically modified plant. Further provided herein is a genetically altered plant genome derived from the genetically modified plant, the genetically altered plant genome comprising the germplasm comprising the MiMe alleles of the genetically modified plant.

In some embodiments, provided herein is a genetically modified plant, plant part, or plant cell having a partially-complemented MiMe genotype. In some embodiments, the genetically modified plant, plant part, or plant cell has a partially-complemented MiMe genotype comprising: (a) only MiMe alleles at one or more MiMe loci of a first MiMe component; (b) one or more MiMe alleles and one or more non-MiMe alleles at a first MiMe locus of a second MiMe component, and one or more MiMe alleles and one or more non-MiMe alleles at a second MiMe locus of the second MiMe component; and (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, the first MiMe component is a component of sister chromatid cohesion during the first division of meiosis. In some variations, the one or more MiMe loci of the first MiMe component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first MiMe locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1. In some embodiments, the genetically modified plant, plant part, or plant cell is a maize plant, a maize plant part, or a maize plant cell. In some embodiments, the genetically modified plant, plant part, or plant cell is a potato plant, a potato plant part, or a potato plant cell. Also provided herein is germplasm of the genetically modified plant, the germplasm comprising the partially-complemented MiMe genotype. Further provided herein is a genetically altered plant genome derived from the genetically modified plant, the genetically altered plant genome comprising the germplasm comprising the partially-complemented MiMe genotype.

In some embodiments, provided herein is a genetically modified plant, plant part, or plant cell having a partially-complemented MiMe genotype comprising: (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiment, the one or more MiMe loci of the component of progression through the first division of meiosis comprise PS1, JASON, or a combination thereof. In some embodiments, the genetically modified plant, plant part, or plant cell is a maize plant, a maize plant part, or a maize plant cell. In some embodiments, the genetically modified plant, plant part, or plant cell is a potato plant, a potato plant part, or a potato plant cell. Also provided herein is germplasm of the genetically modified plant, the germplasm comprising the partially-complemented MiMe genotype. Further provided herein is a genetically altered plant genome derived from the genetically modified plant, the genetically altered plant genome comprising the germplasm comprising the partially-complemented MiMe genotype.

In some embodiments, provided herein is a genetically modified plant, plant part, or plant cell having a partially-complemented MiMe genotype comprising: (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of sister chromatid cohesion during the first division of meiosis comprise REC8, SWITCH1/DYAD, or a combination thereof. In yet additional variations of the foregoing embodiments, the one or more MiMe loci of the component of progression through the second division of meiosis comprise OSD1, CYCA1, TDM1, PC1, PC2, FC, or any combination thereof. In still additional variations of the foregoing embodiments, the one or more MiMe loci of the component of progression through the first division of meiosis comprise PS1, JASON, or a combination thereof. In some embodiments, the genetically modified plant, plant part, or plant cell is a maize plant, a maize plant part, or a maize plant cell. In some embodiments, the genetically modified plant, plant part, or plant cell is a potato plant, a potato plant part, or a potato plant cell. Also provided herein is germplasm of the genetically modified plant, the germplasm comprising the partially-complemented MiMe genotype. Further provided herein is a genetically altered plant genome derived from the genetically modified plant, the genetically altered plant genome comprising the germplasm comprising the partially-complemented MiMe genotype.

In some embodiments which may be combined with any of the preceding embodiments, the genetically modified plant, plant part, or plant cell is diploid, triploid, tetraploid, pentaploid, hexaploid, heptaploid, or octoploid. In additional embodiments which may be combined with any of the preceding embodiments, the genetically modified plant, plant part, or plant cell comprises two, three, four, or more haplotypes of the same or related species of plant. In yet additional embodiments, which may be combined with any of the previous embodiments, the genetically modified plant, plant part, or plant cell is from potato, maize, banana, plantain, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussels sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine and mandarin, clementine, coffee, cola, hazelnut, saffron, melon and cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycress, green bean, bean, ground cherry, pine, pistachio nut, pea, turf grass, poplar, apricot, plum and prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli or cauliflower. In certain embodiments, which may be combined with the foregoing embodiments, the genetically modified plant, plant part, or plant cell is from a parthenocarpic plant. In some embodiments, which may be combined with the foregoing embodiments, the genetically modified plant part is a non-regenerable plant part. In some embodiments which may be combined with the foregoing embodiments, the genetically modified plant cell is a non-regenerable plant cell. In certain embodiments, which may be combined with any of the previous embodiments, the plant part is a flower, a pistil, a leaf, a stem, a petiole, a cutting, a tissue, a seed coat, an ovule, pollen, a tuber, a root, a rootstock, a scion, a fruit, a cotyledon, a hypocotyl, a protoplast, an embryo, an anther, or a portion thereof. In one variation of the foregoing embodiments, the genetically modified plant part is a seedless fruit.

In certain embodiments, which may be combined with any of the previous embodiments, the maize plant part is a flower, a pistil, a stamen, a leaf, a stem, a cutting, a tissue, a seed coat, an ovule, pollen, a root, a rootstock, a scion, a pericarp, a cotyledon, a hypocotyl, a protoplast, an embryo, an endosperm, an anther, a seed, a cob, a glume, a husk, a leaf sheath, a ligule, a trichome, or a portion thereof.

In certain embodiments, which may be combined with any of the previous embodiments, the potato plant part is a flower, a pistil, a leaf, a stem, a petiole, a cutting, a tissue, a seed coat, an ovule, a microspore, pollen, a tuber, a stolon, a meristem, a root, a rootstock, a scion, a fruit, a cotyledon, a hypocotyl, a protoplast, an embryo, an anther, or a portion thereof.

In some embodiments, provided herein is a processed plant product derived from the genetically modified plant, plant part, or plant cell of any one of the preceding embodiments. In certain embodiments, the processed plant product comprises a detectable amount of the one or more MiMe alleles of the genetically modified plant, plant part, or plant cell. In certain embodiments, the product is selected from the group consisting of plant biomass, oil, meal, food starch, syrup, animal feed, flour, flakes, bran, lint, hulls, processed seed, puree, juice, juice concentrate, pulp, pomace, preserve, or sauce. In some embodiments, the processed plant product is non-regenerable. In certain embodiments, the processed plant product contains sufficient nucleic acid (e.g., DNA or RNA) and/or protein material from the genetically modified plant, plant part, or plant cell to detect nucleic acid and/or protein sequences corresponding to the three haplotypes, the one or more genetic modifications resulting in decreased expression of one or more Mime loci, or both.

In some embodiments, provided herein is a processed maize product derived from the genetically modified maize plant, plant part, or plant cell of any one of the preceding embodiments. In certain embodiments, the processed maize product comprises a detectable amount of the one or more MiMe alleles of the genetically modified maize plant, plant part, or plant cell. In certain embodiments, the maize product is selected from the group consisting of plant biomass, oil, meal, food starch, syrup, animal feed, flour, flakes, bran, lint, hulls, processed seed, puree, juice, juice concentrate, pulp, pomace, preserve, or sauce. In some embodiments, the processed maize product is non-regenerable. In certain embodiments, the processed maize product contains sufficient nucleic acid (e.g., DNA or RNA) and/or protein material from the genetically modified maize plant, plant part, or plant cell to detect nucleic acid and/or protein sequences corresponding to the three haplotypes, the one or more genetic modifications resulting in decreased expression of one or more MiMe loci, or both.

In some embodiments, provided herein is a processed potato product derived from the genetically modified potato plant, plant part, or plant cell of any one of the preceding embodiments. In certain embodiments, the processed potato product comprises a detectable amount of the one or more MiMe alleles of the genetically modified potato plant, plant part, or plant cell. In certain embodiments, the potato product is selected from the group consisting of plant biomass, oil, meal, food starch, syrup, animal feed, flour, flakes, bran, lint, hulls, processed seed, puree, juice, juice concentrate, pulp, pomace, preserve, sauce, chips, crisps, or French fries. In some embodiments, the processed potato product is non-regenerable. In certain embodiments, the processed potato product contains sufficient nucleic acid (e.g., DNA or RNA) and/or protein material from the genetically modified potato plant, plant part, or plant cell to detect nucleic acid and/or protein sequences corresponding to the three haplotypes, the one or more genetic modifications resulting in decreased expression of one or more MiMe loci, or both.

In some embodiments, provided herein is a processed plant product derived from a genetically modified plant, plant part, or plant cell comprising: i) three or more haplotypes; and ii) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In other embodiments, provided herein is a processed plant product derived from a genetically modified plant, plant part, or plant cell comprising: i) three or more haplotypes; and ii) a partial MiMe genotype comprising: (a) one or more MiMe alleles conferring decreased expression of one or more MiMe loci of each of a first, second, and third MiMe component; and (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the one or more MiMe loci of the component of progression through the second division of meiosis comprise OSD1, CYCA1, TDM1, PC1, PC2, FC, or any combination thereof. In certain embodiments, the processed plant product comprises sufficient nucleic acid (e.g., DNA or RNA) and/or protein material from the genetically modified plant, plant part, or plant cell to detect nucleic acid and/or protein sequences corresponding to the three haplotypes, the MiMe alleles, or both in the processed plant product. In certain embodiments, the processed plant product is non-regenerable. In some embodiments, the processed plant product is a processed maize product derived from a genetically modified maize plant, maize plant part, or maize plant cell. In some embodiments, the processed plant product is a processed potato product derived from a genetically modified potato plant, potato plant part, or potato plant cell.

In some embodiments, provided herein is a processed plant product derived from a genetically modified plant, plant part, or plant cell comprising: i) three or more haplotypes; and ii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components. In other embodiments, provided herein is a processed plant product derived from a genetically modified plant, plant part, or plant cell comprising: i) three or more haplotypes; and ii) a partial MiMe genotype comprising: (a) one or more MiMe alleles conferring decreased expression of one or more MiMe loci of each of the first and second MiMe component; and (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In other variations, the one or more MiMe loci of the component of progression through the first division of meiosis comprise PS1, JASON, or a combination thereof. In certain embodiments, the processed plant product comprises sufficient nucleic acid (e.g., DNA or RNA) and/or protein material from the genetically modified plant, plant part, or plant cell to detect nucleic acid and/or protein sequences corresponding to the three haplotypes, the MiMe alleles, or both in the processed plant product. In certain embodiments, the processed plant product is non-regenerable. In some embodiments, the processed plant product is a processed maize product derived from a genetically modified maize plant, maize plant part, or maize plant cell. In some embodiments, the processed plant product is a processed potato product derived from a genetically modified potato plant, potato plant part, or potato plant cell.

In some embodiments, provided herein is a processed plant product derived from a genetically modified plant, plant part, or plant cell comprising: (i) at least a first and second haplotype, each comprising one or more MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of sister chromatid cohesion during the first division of meiosis comprise REC8, SWITCH1/DYAD, or a combination thereof. In additional variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In yet additional variations, the MiMe locus of the component of progression through the first division of meiosis of the third haplotype is PS1 or JASON. In still additional variations, the one or more MiMe loci of the component of progression through the second division of meiosis of the first and second haplotype comprise OSD1, CYCA1, TDM1, PC1, PC2, FC, or any combination thereof. In yet additional variations, the locus of the component of progression through the second division of meiosis of the third haplotype is OSD1, CYCA1, TDM1, PC1, PC2, or FC. In certain embodiments, the processed plant product comprises sufficient nucleic acid (e.g., DNA or RNA) and/or protein material from the genetically modified plant, plant part, or plant cell to detect nucleic acid and/or protein sequences corresponding to the three haplotypes, the MiMe alleles, or both in the processed plant product. In certain embodiments, the processed plant product is non-regenerable. In some embodiments, the processed plant product is a processed maize product derived from a genetically modified maize plant, maize plant part, or maize plant cell. In some embodiments, the processed plant product is a processed potato product derived from a genetically modified potato plant, potato plant part, or potato plant cell.

In some embodiments, provided herein is a processed plant product derived from a genetically modified plant, plant part, or plant cell comprising: (i) at least a first and second haplotype, each comprising one or more MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components, and (ii) at least a third haplotype comprising (a) a MiMe allele conferring decreased expression of a MiMe locus of a component of progression through the first division of meiosis, or (b) a MiMe allele conferring decreased expression of a MiMe locus of a component of progression through the second division of meiosis. In some variations, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations, the one or more MiMe loci of the component of progression through the first division of meiosis of the first and second haplotype comprise PS1, JASON, or a combination thereof. In yet additional variations, the MiMe locus of the component of progression through the first division of meiosis of the third haplotype is PS1 or JASON. In still additional variations, the MiMe locus of the component of progression through the second division of meiosis of the third haplotype is OSD1, CYCA1, TDM1, PC1, PC2 or FC. In certain embodiments, the processed plant product comprises sufficient nucleic acid (e.g., DNA or RNA) and/or protein material from the genetically modified plant, plant part, or plant cell to detect nucleic acid and/or protein sequences corresponding to the three haplotypes, the MiMe alleles, or both in the processed plant product. In certain embodiments, the processed plant product is non-regenerable. In some embodiments, the processed plant product is a processed maize product derived from a genetically modified maize plant, maize plant part, or maize plant cell. In some embodiments, the processed plant product is a processed potato product derived from a genetically modified potato plant, potato plant part, or potato plant cell.

In some embodiments, provided herein is a processed plant product derived from a genetically modified plant, plant part, or plant cell having a partially-complemented Mime genotype. In some embodiments, a processed plant product derived from a genetically modified plant, plant part, or plant cell having a partially-complemented Mime genotype comprising: (a) only MiMe alleles at one or more Mime loci of a first Mime component; (b) one or more MiMe alleles and one or more non-MiMe alleles at a first Mime locus of a second MiMe component, and one or more MiMe alleles and one or more non-MiMe alleles at a second MiMe locus of the second MiMe component; and (c) either (i) only MiMe alleles at one or more MiMe loci of a third Mime component, or (ii) one or more Mime alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components. In some embodiments, the first MiMe component is a component of sister chromatid cohesion during the first division of meiosis. In some variations, the one or more Mime loci of the first Mime component comprise REC8, SWITCH1/DYAD, or a combination thereof. In one variation, the MiMe locus of the first MiMe component is REC8. In certain embodiments, the second MiMe component is a component of DNA double strand breakage during meiotic recombination. In some variations, the first MiMe locus and the second MiMe locus of the second MiMe component comprise PAIR1, SPO11-1, SPO11-2, PRD1, PRD2, DFO, MTOPVIB, DSY1, SY1, SY2, SY3, SY4, or any combination thereof. In one variation, the first MiMe locus of the second MiMe component is PAIR1 and the second MiMe locus of the second MiMe component is SPO11-1. In further embodiments, the third MiMe component is a component of progression through the second division of meiosis. In some embodiments, the partially-complemented MiMe genotype comprises only MiMe alleles at one or more MiMe loci of the third MiMe component. In some variations, the one or more MiMe loci of the third MiMe component comprise OSD1, CYCA1, TDM1, PC1, PC2, FC, or any combination thereof. In one variation, the MiMe locus of the third MiMe component is OSD1. In other embodiments, the partially-complemented MiMe genotype comprises one or more MiMe alleles and one or more non-MiMe alleles at a first Mime locus of the third MiMe component, and one or more MiMe alleles and one or more non-MiMe alleles at a second MiMe locus of the third MiMe component. In some variations, the first MiMe locus and the second MiMe locus of the third MiMe component comprise OSD1, CYCA1, TDM1, PC1, PC2, FC, or any combination thereof. In one embodiment, the partially-complemented MiMe genotype comprises only MiMe alleles at one or more MiMe loci of the third MiMe component, wherein the one or more MiMe loci having only MiMe alleles of the first MiMe component comprise REC8, the first MiMe locus of the second MiMe component is PAIR1, the second MiMe locus of the second MiMe component is SPO11-1, and the one or more MiMe loci having only MiMe alleles of the third MiMe component comprise OSD1. In certain embodiments which may be combined with the foregoing embodiments, the processed plant product is derived from a seedless fruit. In some variations of the foregoing embodiments, the processed plant product is non-regenerable. In some embodiments, the processed plant product is a processed maize product derived from a genetically modified maize plant, maize plant part, or maize plant cell. In some embodiments, the processed plant product is a processed potato product derived from a genetically modified potato plant, potato plant part, or potato plant cell.

In some embodiments, provided herein is a processed plant product derived from a genetically modified plant, plant part, or plant cell having a partially-complemented MiMe genotype comprising: (a) only MiMe alleles at one or more MiMe loci of a first MiMe component; and (b) one or more MiMe alleles and one or more non-MiMe alleles at a first MiMe locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiment, the one or more MiMe loci of the component of progression through the first division of meiosis comprise PS1, JASON, or a combination thereof. In certain embodiments which may be combined with the foregoing embodiments, the processed plant product is derived from a seedless fruit. In some variations of the foregoing embodiments, the processed plant product is non-regenerable. In some embodiments, the processed plant product is a processed maize product derived from a genetically modified maize plant, maize plant part, or maize plant cell. In some embodiments, the processed plant product is a processed potato product derived from a genetically modified potato plant, potato plant part, or potato plant cell.

In some embodiments, provided herein is a processed plant product derived from a genetically modified plant, plant part, or plant cell having a partially-complemented MiMe genotype comprising: (a) only MiMe alleles at one or more MiMe loci of a first MiMe component, wherein the first MiMe component is a component of DNA double strand breakage during meiotic recombination; (b) one or more MiMe alleles and one or more non-MiMe alleles at one or more MiMe loci of a second MiMe component; (c) one or more MiMe alleles and one or more non-MiMe alleles at one or more MiMe loci of a third MiMe component; and (d) one or more MiMe alleles and one or more non-MiMe alleles at one or more MiMe loci of a fourth MiMe component, wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components. In some variations of the foregoing embodiments, the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof. In additional variations of the foregoing embodiments, the one or more MiMe loci of the component of sister chromatid cohesion during the first division of meiosis comprise REC8, SWITCH1/DYAD, or a combination thereof. In yet additional variations of the foregoing embodiments, the one or more MiMe loci of the component of progression through the second division of meiosis comprise OSD1, CYCA1, TDM1, PC1, PC2, FC, or any combination thereof. In still additional variations of the foregoing embodiments, the one or more MiMe loci of the component of progression through the first division of meiosis comprise PS1, JASON, or a combination thereof. In certain embodiments which may be combined with the foregoing embodiments, the processed plant product is derived from a seedless fruit. In some variations of the foregoing embodiments, the processed plant product is non-regenerable. In some embodiments, the processed plant product is a processed maize product derived from a genetically modified maize plant, maize plant part, or maize plant cell. In some embodiments, the processed plant product is a processed potato product derived from a genetically modified potato plant, potato plant part, or potato plant cell.

In some embodiments, provided herein is a genetically modified plant, plant part, or plant cell, or a processed plant product derived therefrom, having a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494. In some variations, the genetically modified plant, plant part, or plant cell is a genetically modified maize plant, plant part, or plant cell, optionally wherein the one or more OSD1 loci comprise OSD1-1, OSD1-2, and/or OSD1-3, and/or the one or more PAIR1 loci comprise PAIR1-1 and/or PAIR1-2. In certain variations, the genetically modified plant, plant part, or plant cell is a genetically modified maize plant, plant part, or plant cell and the one or more OSD1 loci comprise OSD1-1 and OSD1-2. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) one or more osd1 alleles, each comprising a polynucleotide sequence selected from the group consisting of SEQ ID NOS: 448 and 449. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more pair1 alleles, each comprising a polynucleotide sequence selected from the group consisting of SEQ ID NOs: 461, 462, and 463. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more OSD1 loci comprise OSD1-1 and/or OSD1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein is a genetically modified plant, plant part, or plant cell, or a processed plant product derived therefrom, having a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494. In some variations, the genetically modified plant, plant part, or plant cell is a genetically modified maize plant, plant part, or plant cell, optionally wherein the one or more OSD1 loci comprise OSD1-1, OSD1-2, and/or OSD1-3, and/or the one or more PAIR1 loci comprise PAIR1-1 and/or PAIR1-2. In certain variations, the genetically modified plant, plant part, or plant cell is a genetically modified maize plant, plant part, or plant cell and the one or more OSD1 loci comprise OSD1-1 and OSD1-2. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) one or more osd1 alleles, each comprising a polynucleotide sequence selected from the group consisting of SEQ ID NOS: 448 and 449. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more pair1 alleles, each comprising a polynucleotide sequence selected from the group consisting of SEQ ID NOs: 461, 462, and 463. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more OSD1 loci comprise OSD1-1 and/or OSD1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial Mime genotype.

In some embodiments, provided herein is a genetically modified plant, plant part, or plant cell, or a processed plant product derived therefrom, having a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally wherein the one or more OSD1 loci comprise OSD1-1, OSD1-2, and/or OSD1-3. In certain variations, the genetically modified plant, plant part, or plant cell is a genetically modified maize plant, plant part, or plant cell and the one or more OSD1 loci comprise OSD1-1 and OSD1-2. In some variations, the genetically modified plant, plant part, or plant cell is a genetically modified maize plant, plant part, or plant cell, optionally comprising a) a rec8 allele comprising the polynucleotide sequence of SEQ ID NO: 450; b) one or more osd1 alleles, each comprising a polynucleotide sequence selected from the group consisting of SEQ ID NOS: 448 and 449; and/or c) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more OSD1 loci comprise OSD1-1 and/or OSD1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein a genetically modified plant, plant part, or plant cell, or a processed plant product derived therefrom, having a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally wherein the one or more OSD1 loci comprise OSD1-1, OSD1-2, and/or OSD1-3. In certain variations, the genetically modified plant, plant part, or plant cell is a genetically modified maize plant, plant part, or plant cell and the one or more OSD1 loci comprise OSD1-1 and OSD1-2. In some variations, the genetically modified plant, plant part, or plant cell is a genetically modified maize plant, plant part, or plant cell, optionally comprising a) a rec8 allele comprising the polynucleotide sequence of SEQ ID NO: 450; b) one or more osd1 alleles, each comprising a polynucleotide sequence selected from the group consisting of SEQ ID NOS: 448 and 449; and/or c) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; and/or c) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more OSD1 loci comprise OSD1-1 and/or OSD1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial MiMe genotype.

In some embodiments, provided herein a genetically modified plant, plant part, or plant cell, or a processed plant product derived therefrom, having a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally wherein the one or more CYCA1 loci comprise CYCA1-1, CYCA1-2, and/or CYCA1-3. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; b) one or more spo11-1 alleles, each comprising a polynucleotide sequence selected from the group consisting of SEQ ID NOs: 464, 465, 466, 470, 471, 476-479, 484, 485, 490, 491, 495, and 496; and/or c) one or more cyca1 alleles, each comprising a polynucleotide sequence selected from the group consisting of SEQ ID NOs: 480, 481, 486, and 487. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein is a genetically modified plant, plant part, or plant cell, or a processed plant product derived therefrom, having a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally wherein the one or more CYCA1 loci comprise CYCA1-1, CYCA1-2, and/or CYCA1-3. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; b) one or more spo11-1 alleles, each comprising a polynucleotide sequence selected from the group consisting of SEQ ID NOs: 464, 465, 466, 470, 471, 476-479, 484, 485, 490, 491, 495, and 496; and/or c) one or more cyca1 alleles, each comprising a polynucleotide sequence selected from the group consisting of SEQ ID NOs: 480, 481, 486, and 487. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial MiMe genotype.

In some embodiments, provided herein a genetically modified plant, plant part, or plant cell, or a processed plant product derived therefrom, having a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally wherein the one or more CYCA1 loci comprise CYCA1-1, CYCA1-2, and/or CYCA1-3. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more cyca1 alleles, each comprising a polynucleotide sequence selected from the group consisting of SEQ ID NOs: 480, 481, 486, and 487. In some variations, the genetically modified plant, plant part, or plant cell is a genetically modified maize plant, plant part, or plant cell, optionally wherein the one or more PAIR1 loci comprise PAIR1-1 and/or PAIR1-2. In some variations, the genetically modified plant, plant part, or plant cell is a genetically modified maize plant, plant part, or plant cell, optionally comprising a rec8 allele comprising the polynucleotide sequence of SEQ ID NO: 450. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein a genetically modified plant, plant part, or plant cell, or a processed plant product derived therefrom, having a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally wherein the one or more CYCA1 loci comprise CYCA1-1, CYCA1-2, and/or CYCA1-3. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more cyca1 alleles, each comprising a polynucleotide sequence selected from the group consisting of SEQ ID NOs: 480, 481, 486, and 487. In some variations, the genetically modified plant, plant part, or plant cell is a genetically modified maize plant, plant part, or plant cell, optionally wherein the one or more PAIR1 loci comprise PAIR1-1 and/or PAIR1-2. In some variations, the genetically modified plant, plant part, or plant cell is a genetically modified maize plant, plant part, or plant cell, optionally comprising a rec8 allele comprising the polynucleotide sequence of SEQ ID NO: 450. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial MiMe genotype.

In some embodiments, provided herein is a genetically modified plant, plant part, or plant cell, or a processed plant product derived therefrom, having a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more TDM1 loci comprise TDM1-1 and/or TDM1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein is a genetically modified plant, plant part, or plant cell, or a processed plant product derived therefrom, having a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more TDM1 loci comprise TDM1-1 and/or TDM1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial MiMe genotype.

In some embodiments, provided herein is a genetically modified plant, plant part, or plant cell, or a processed plant product derived therefrom, having a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising one or more rec8 alleles, each comprising a polynucleotide sequence selected from the group consisting of SEQ ID NOs: 465, 466, 468, 469, 474, 475, 482, 483, 488, 489, 493, and 494. In some variations, the genetically modified plant, plant part, or plant cell is a genetically modified maize plant, plant part, or plant cell, optionally wherein the one or more PAIR1 loci comprise PAIR1-1 and/or PAIR1-2. In some variations, the genetically modified plant, plant part, or plant cell is a genetically modified maize plant, plant part, or plant cell, optionally comprising a rec8 allele comprising the polynucleotide sequence of SEQ ID NO: 450. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more TDM1 loci comprise TDM1-1 and/or TDM1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein is a genetically modified plant, plant part, or plant cell, or a processed plant product derived therefrom, having a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising one or more rec8 alleles, each comprising a polynucleotide sequence selected from the group consisting of SEQ ID NOs: 465, 466, 468, 469, 474, 475, 482, 483, 488, 489, 493, and 494. In some variations, the genetically modified plant, plant part, or plant cell is a genetically modified maize plant, plant part, or plant cell, optionally wherein the one or more PAIR1 loci comprise PAIR1-1 and/or PAIR1-2. In some variations, the genetically modified plant, plant part, or plant cell is a genetically modified maize plant, plant part, or plant cell, optionally comprising a rec8 allele comprising the polynucleotide sequence of SEQ ID NO: 450. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more pair1 alleles, each comprising a polynucleotide sequence selected from the group consisting of SEQ ID NOs: 461, 462, and 463. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more TDM1 loci comprise TDM1-1 and/or TDM1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial MiMe genotype.

In some embodiments, provided herein is a genetically modified plant, plant part, or plant cell, or a processed plant product derived therefrom, having a complete MiMe genotype comprising (i) an os allele, wherein the genetically modified plant, plant part, or plant cell is homozygous for the os allele, (ii) only Mime alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein is a genetically modified plant, plant part, or plant cell, or a processed plant product derived therefrom, having a partial MiMe genotype comprising (i) an os allele, wherein the genetically modified plant, plant part, or plant cell is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial MiMe genotype.

In some embodiments, provided herein is a genetically modified plant, plant part, or plant cell, or a processed plant product derived therefrom, having a complete MiMe genotype comprising (i) a ps allele, wherein the genetically modified plant, plant part, or plant cell is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the complete MiMe genotype. Further provided herein is a genetically altered plant genome derived from the genetically modified plant, the genetically altered plant genome comprising the complete MiMe genotype.

In some embodiments, provided herein is a genetically modified plant, plant part, or plant cell, or a processed plant product derived therefrom, having a partial MiMe genotype comprising (i) a ps allele, wherein the genetically modified plant, plant part, or plant cell, is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partial MiMe genotype. Further provided herein is a genetically altered plant genome derived from the genetically modified plant, the genetically altered plant genome comprising the partial MiMe genotype.

In some embodiments, provided herein is a genetically modified plant, plant part, or plant cell, or a processed plant product derived therefrom, having a partially complemented MiMe genotype comprising (i) only MiMe alleles at one or more OSD1 loci, wherein each of the Mime alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more only MiMe alleles at one or more SPO11-1 loci, wherein each of the MiMe alleles at the SPO11-1 locus comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally wherein the one or more OSD1 loci comprise OSD1-1, OSD1-2, and/or OSD1-3, and/or the one or more PAIR1 loci comprise PAIR1-1 and/or PAIR1-2. In certain variations, the genetically modified plant, plant part, or plant cell is a genetically modified maize plant, plant part, or plant cell and the one or more OSD1 loci comprise OSD1-1 and OSD1-2. In some variations, the genetically modified plant, plant part, or plant cell is a genetically modified maize plant, plant part, or plant cell, optionally comprising a) a rec8 allele comprising the polynucleotide sequence of SEQ ID NO: 450; b) one or more osd1 alleles, each comprising a polynucleotide sequence selected from the group consisting of SEQ ID NOS: 448 and 449; and/or c) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; b) one or more osd1 alleles, each comprising a polynucleotide sequence selected from the group consisting of SEQ ID NOs: 452, 453, 458, 459, and 463; c) one or more pair1 alleles, each comprising a polynucleotide sequence selected from the group consisting of SEQ ID NOs: 461, 462, and 463; and/or d) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more OSD1 loci comprise OSD1-1 and/or OSD1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partially complemented MiMe genotype. Further provided herein is a genetically altered plant genome derived from the genetically modified plant, the genetically altered plant genome comprising the partially complemented MiMe genotype.

In some embodiments, provided herein is a genetically modified plant, plant part, or plant cell, or a processed plant product derived therefrom, having a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally wherein the one or more CYCA1 loci comprise CYCA1-1, CYCA1-2, and/or CYCA1-3. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; b) one or more cyca1 alleles, each comprising a polynucleotide sequence selected from the group consisting of SEQ ID NOs: 480, 481, 486, and 487; and/or c) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally wherein the one or more PAIR1 loci comprise PAIR1-1 and/or PAIR1-2. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; b) one or more pair1 alleles, each comprising a polynucleotide sequence selected from the group consisting of SEQ ID NOs: 461, 462, and 463; and/or c) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partially complemented MiMe genotype. Further provided herein is a genetically altered plant genome derived from the genetically modified plant, the genetically altered plant genome comprising the partially complemented MiMe genotype.

In some embodiments, provided herein is a genetically modified plant, plant part, or plant cell, or a processed plant product derived therefrom, having a partially complemented MiMe genotype comprising (i) only MiMe alleles at one or more TDM1 loci, wherein each of the Mime alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally wherein the one or more PAIR1 loci comprise PAIR1-1 and/or PAIR1-2. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; b) one or more pair1 alleles, each comprising a polynucleotide sequence selected from the group consisting of SEQ ID NOs: 461, 462, and 463; and/or c) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some variations, the genetically modified plant, plant part, or plant cell is a genetically modified soybean plant, plant part, or plant cell, optionally wherein the one or more TDM1 loci comprise TDM1-1 and/or TDM1-2. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partially complemented MiMe genotype. Further provided herein is a genetically altered plant genome derived from the genetically modified plant, the genetically altered plant genome comprising the partially complemented MiMe genotype.

In some embodiments, provided herein is a genetically modified plant, plant part, or plant cell, or a processed plant product derived therefrom, having a partially complemented MiMe genotype comprising (i) a ps allele, wherein the genetically modified plant, plant part, or plant cell is heterozygous for the ps allele, (ii) an os allele, wherein the genetically modified plant, plant part, or plant cell is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; and/or b) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partially complemented MiMe genotype. Further provided herein is a genetically altered plant genome derived from the genetically modified plant, the genetically altered plant genome comprising the partially complemented MiMe genotype.

In some embodiments, provided herein is a genetically modified plant, plant part, or plant cell, or a processed plant product derived therefrom, having a partially complemented MiMe genotype comprising (i) an os allele, wherein the genetically modified plant, plant part, or plant cell is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1. In certain embodiments, the genetically modified plant, plant part, or plant cell, or processed plant product derived therefrom, is triploid, tetraploid, pentaploid, hexaploid, heptaploid, or octoploid. In some variations, the genetically modified plant, plant part, or plant cell is a genetically modified potato plant, plant part, or plant cell, optionally wherein the one or more CYCA1 loci comprise CYCA1-1, CYCA1-2, and/or CYCA1-3. In some variations, the genetically modified plant, plant part, or plant cell is a genetically modified potato plant, plant part, or plant cell, optionally comprising a) one or more rec8 alleles, each comprising a polynucleotide sequence selected from the group consisting of SEQ ID NOs: 465, 466, 468, 469, 474, 475, 482, 483, 488, 489, 493, and 494; b) one or more cyca1 alleles, each comprising a polynucleotide sequence selected from the group consisting of SEQ ID NOs: 480, 481, 486, and 487; and/or c) one or more spo11-1 alleles, each comprising a polynucleotide sequence selected from the group consisting of SEQ ID NOs: 464, 465, 466, 470, 471, 476-479, 484, 485, 490, 491, 495, and 496. In some variations, the genetically modified plant, plant part, or plant cell is a genetically modified maize plant, plant part, or plant cell, optionally comprising a) a rec8 allele comprising the polynucleotide sequence of SEQ ID NO: 450; and/or b) a spo11-1 allele comprising the polynucleotide sequence of SEQ ID NO: 451. In some variations, the genetically modified plant, plant part, or plant cell is a genetically modified Arabidopsis plant, plant part, or plant cell, optionally comprising a) one or more rec8 alleles, each comprising a polynucleotide sequence selected from the group consisting of SEQ ID NOs: 454, 455, 459, 460, and 461; and/or b) one or more spo11-1 alleles, each comprising a polynucleotide sequence selected from the group consisting of SEQ ID NOs: 456 and 457. In some variations, the genetically modified plant, plant part, or plant cell is a genetically modified tomato plant, plant part, or plant cell. In some embodiments, the genetically modified plant, plant part, or plant cell is a genetically modified banana plant, plant part, or plant cell. In some variations, the genetically modified plant, plant part, or plant cell is a genetically modified blueberry plant, plant part, or plant cell. In some variations, the genetically modified plant, plant part, or plant cell is a genetically modified blackberry plant, plant part, or plant cell. Also provided herein is germplasm of the genetically modified plant, the germplasm comprising the partially complemented MiMe genotype. Further provided herein is a genetically altered plant genome derived from the genetically modified plant, the genetically altered plant genome comprising the partially complemented MiMe genotype.

SEQUENCE ALIGNMENT 1
REC8 protein sequences from dicotyledonous plants.
REC8 Dicots
Homologs identified by BLAST of Arabidopsis thaliana AT5G05490.1 from [symbol] map to dicot
NCBI RefSeq, UniProtKB, Vaccinium corymbosum Draper v1.0, Rubus occidentalis
v3.0, Citrullus lanatus (97103) v2.0, and Citrullus lanatus (Charleston Gray) v1.0 protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |
| | 210 220 230 240 250 260 270 280 290 300 |
| | ----:----\|----:----\|----:----\|----:----\|----:----\|----:----\|----:----\|----:----\|----:----\|----:----\| |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |
| | 310 320 330 340 350 360 370 380 390 400 |
| | ----:----\|----:----\|----:----\|----:----\|----:----\|----:----\|----:----\|----:----\|----:----\|----:----\| |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |
| | 410 420 430 440 450 460 470 480 490 500 |
| | ----:----\|----:----\|----:----\|----:----\|----:----\|----:----\|----:----\|----:----\|----:----\|----:----\| |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |
| | 510 520 530 540 550 560 570 580 590 600 |
| | ----:----\|----:----\|----:----\|----:----\|----:----\|----:----\|----:----\|----:----\|----:----\|----:----\| |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |
| | 610 620 630 640 650 660 670 680 690 700 |
| | ----:----\|----:----\|----:----\|----:----\|----:----\|----:----\|----:----\|----:----\|----:----\|----:----\| |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |
| | 710 720 730 740 |
| | ----:----\|----:----\|----:----\|----:----\|----:-- |
| Consensus | [symbol] |
| Gossypium barbadense: (UniProt: [symbol] ) | [symbol] |
| Arabidopsis thaliana: (UniProt: [symbol] ) | [symbol] |
| Rubus occidentalis: ( [symbol] ) | [symbol] |
| Citrullus lanatus: ( [symbol] ) | [symbol] |
| Prunus avium: (UniProt: [symbol] ) | [symbol] |
| Vaccinium corymbosum: ( [symbol] ) | [symbol] |
| Solanum lycopersicum: (RefSeq: XP_025887480.1) | [symbol] |
| Solanum tuberosum: (RefSeq: XP_006347252.1) | [symbol] |

[symbol]

indicates data missing or illegible when filed

TABLE 1
Percent identities of REC8 protein sequences from dicotyledonous plants.

| | | | | | | Vaccinium | |
|---|---|---|---|---|---|---|---|
| | | | | | | corymbosum: | Solanum |
| | Gossypium | Arabidopsis | | | Prunus | (VaccDscaff20- | lycopersicum: |
| REC8 | barbadense: | thaliana: | Rubus | Citrullus | avium: | snap-gene- | (RefSeq: |
| Dicot % | (UniProt: | (UniProt: | occidentalis: | lanatus: | (UniProt: | 38.40- | XP_{—} |
| ID | A0A5J5VWS8) | Q9S7T7) | (Ro05G04582) | (ClCG07G004460.1) | A0A6P5RV91) | mRNA-1) | 025887480.1) |
| Arabidopsis | 33.073 | | | | | | |
| thaliana: | | | | | | | |
| (UniProt: Q9S7T7) | | | | | | | |
| Rubus | 35.349 | 37.349 | | | | | |
| occidentalis: | | | | | | | |
| (Ro05G04582) | | | | | | | |
| Citrullus | 34.967 | 34.202 | 36.687 | | | | |
| lanatus: | | | | | | | |
| (ClCG07G004460.1) | | | | | | | |
| Prunus | 38.915 | 45.092 | 50 | 45.938 | | | |
| avium: (UniProt: | | | | | | | |
| A0A6P5RV91) | | | | | | | |
| Vaccinium | 39.905 | 44.275 | 49.158 | 48.742 | 60.377 | | |
| corymbosum: | | | | | | | |
| (VaccDscaff20- | | | | | | | |
| snap-gene- | | | | | | | |
| 38.40-mRNA-1) | | | | | | | |
| Solanum | 37.915 | 42.857 | 44.36 | 43.906 | 52.812 | 59.781 | |
| lycopersicum: | | | | | | | |
| (RefSeq: | | | | | | | |
| XP_025887480.1) | | | | | | | |
| Solanum | 33.949 | 39.31 | 40.582 | 40.028 | 48.091 | 54.249 | 85.714 |
| tuberosum: | | | | | | | |
| (RefSeq: | | | | | | | |
| XP_006347252.1) | | | | | | | |

SEQUENCE ALIGNMENT 2
REC8 protein sequences from monocotyledonous plants.
---------------------------------------------------------
REC8 Monocots
Homologs identified by BLAST of Oryza sativa Os05g50430.1 from MSU-Osa1
Release 7 map to monocot NCBI RefSeq, GenBank and UniProtKB protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Aegilops_tauschii_subsp._tauschii:(RefSeq:XP_ [symbol] ) | [symbol] |
| Brachypodium_distachyon:(RefSeq:XP_ [symbol] ) | [symbol] |
| Hordeum_vulgare_subsp._vulgare:(XP_ [symbol] ) | [symbol] |
| Musa_acuminata_subsp._malaccensis:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_021302730.1) | [symbol] |
| Triticum_aestivum:(UniProt: [symbol] ) | [symbol] |
| Zea_mays:(RefSeq:XP_ [symbol] ) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Aegilops_tauschii_subsp._tauschii:(RefSeq:XP_ [symbol] ) | [symbol] |
| Brachypodium_distachyon:(RefSeq:XP_ [symbol] ) | [symbol] |
| Hordeum_vulgare_subsp._vulgare:(XP_ [symbol] ) | [symbol] |
| Musa_acuminata_subsp._malaccensis:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_021302730.1) | [symbol] |
| Triticum_aestivum:(UniProt: [symbol] ) | [symbol] |
| Zea_mays:(RefSeq:XP_ [symbol] ) | [symbol] |
| | 210 220 230 240 250 260 270 280 290 300 |
| | ----:----\|----:----\|----:----\|----:----\|----:----\|----:----\|----:----\|----:----\|----:----\|----:----\| |
| Consensus | [symbol] |
| Aegilops_tauschii_subsp._tauschii:(RefSeq:XP_ [symbol] ) | [symbol] |
| Brachypodium_distachyon:(RefSeq:XP_ [symbol] ) | [symbol] |
| Hordeum_vulgare_subsp._vulgare:(XP_ [symbol] ) | [symbol] |
| Musa_acuminata_subsp._malaccensis:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_021302730.1) | [symbol] |
| Triticum_aestivum:(UniProt: [symbol] ) | [symbol] |
| Zea_mays:(RefSeq:XP_ [symbol] ) | [symbol] |
| | 310 320 330 340 350 360 370 380 390 400 |
| | ----:----\|----:----\|----:----\|----:----\|----:----\|----:----\|----:----\|----:----\|----:----\|----:----\| |
| Consensus | [symbol] |
| Aegilops_tauschii_subsp._tauschii:(RefSeq:XP_ [symbol] ) | [symbol] |
| Brachypodium_distachyon:(RefSeq:XP_ [symbol] ) | [symbol] |
| Hordeum_vulgare_subsp._vulgare:(XP_ [symbol] ) | [symbol] |
| Musa_acuminata_subsp._malaccensis:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_021302730.1) | [symbol] |
| Triticum_aestivum:(UniProt: [symbol] ) | [symbol] |
| Zea_mays:(RefSeq:XP_ [symbol] ) | [symbol] |
| | 410 420 430 440 450 460 470 480 490 500 |
| | ----:----\|----:----\|----:----\|----:----\|----:----\|----:----\|----:----\|----:----\|----:----\|----:----\| |
| Consensus | [symbol] |
| Aegilops_tauschii_subsp._tauschii:(RefSeq:XP_ [symbol] ) | [symbol] |
| Brachypodium_distachyon:(RefSeq:XP_ [symbol] ) | [symbol] |
| Hordeum_vulgare_subsp._vulgare:(XP_ [symbol] ) | [symbol] |
| Musa_acuminata_subsp._malaccensis:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_021302730.1) | [symbol] |
| Triticum_aestivum:(UniProt: [symbol] ) | [symbol] |
| Zea_mays:(RefSeq:XP_ [symbol] ) | [symbol] |
| | 510 520 530 540 550 560 570 580 590 600 |
| | ----:----\|----:----\|----:----\|----:----\|----:----\|----:----\|----:----\|----:----\|----:----\|----:----\| |
| Consensus | [symbol] |
| Aegilops_tauschii_subsp._tauschii:(RefSeq:XP_ [symbol] ) | [symbol] |
| Brachypodium_distachyon:(RefSeq:XP_ [symbol] ) | [symbol] |
| Hordeum_vulgare_subsp._vulgare:(XP_ [symbol] ) | [symbol] |
| Musa_acuminata_subsp._malaccensis:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_021302730.1) | [symbol] |
| Triticum_aestivum:(UniProt: [symbol] ) | [symbol] |
| Zea_mays:(RefSeq:XP_ [symbol] ) | [symbol] |
| | 610 620 630 |
| | ----:----\|----:----\|----:----\|-- |
| Consensus | [symbol] |
| Aegilops_tauschii_subsp._tauschii:(RefSeq:XP_ [symbol] ) | [symbol] |
| Brachypodium_distachyon:(RefSeq:XP_ [symbol] ) | [symbol] |
| Hordeum_vulgare_subsp._vulgare:(XP_ [symbol] ) | [symbol] |
| Musa_acuminata_subsp._malaccensis:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_021302730.1) | [symbol] |
| Triticum_aestivum:(UniProt: [symbol] ) | [symbol] |
| Zea_mays:(RefSeq:XP_ [symbol] ) | [symbol] |

[symbol]

indicates data missing or illegible when filed

TABLE 2
Percent identities of REC8 protein sequences from monocotyledonous plants.

| | | | | Japonica |
|---|---|---|---|---|
| | | | Sorghum | Oryza sativa |
| REC8 | Musa acuminata subsp. | Zea mays: | bicolor: | Group: |
| Monocot % | malaccensis: (RefSeq: | (RefSeq: | (RefSeq: | (RefSeq: |
| ID | XP_018681669.1) | XP_008648328.1) | XP_021302730.1) | XP_015638093.1) |
| Zea mays: | 55.863 | | | |
| (RefSeq: | | | | |
| XP_008648328.1) | | | | |
| Sorghum | 53.71 | 86.721 | | |
| bicolor: (RefSeq: | | | | |
| XP_021302730.1) | | | | |
| Oryza sativa | 52.674 | 67.585 | 68.182 | |
| Japonica | | | | |
| Group: (RefSeq: | | | | |
| XP_015638093.1) | | | | |
| Brachypodium | 55.628 | 72.386 | 72.683 | 70.425 |
| distachyon: | | | | |
| (RefSeq: | | | | |
| XP_003567819.1) | | | | |
| Hordeum vulgare | 51.545 | 64.542 | 65.579 | 63.725 |
| subsp. vulgare: | | | | |
| (XP_044984305.1) | | | | |
| Triticum_aestivum: | 52.903 | 67.585 | 68.821 | 65.964 |
| (UniProt: | | | | |
| A0A3B5Z6B7) | | | | |
| Aegilops tauschii | 53.065 | 68.071 | 68.821 | 67.099 |
| subsp. | | | | |
| tauschii: (RefSeq: | | | | |
| XP_020178692.1) | | | | |

| | | Hordeum | |
|---|---|---|---|
| | Brachypodium | vulgare | Triticum |
| REC8 | distachyon: | subsp. | aestivum: |
| Monocot % | (RefSeq: | vulgare: | (UniProt: |
| ID | XP_003567819.1) | (XP_044984305.1) | A0A3B5Z6B7) |
| Zea mays: | | | |
| (RefSeq: | | | |
| XP_008648328.1) | | | |
| Sorghum | | | |
| bicolor: (RefSeq: | | | |
| XP_021302730.1) | | | |
| Oryza sativa | | | |
| Japonica | | | |
| Group: (RefSeq: | | | |
| XP_015638093.1) | | | |
| Brachypodium | | | |
| distachyon: | | | |
| (RefSeq: | | | |
| XP_003567819.1) | | | |
| Hordeum vulgare | 75.413 | | |
| subsp. vulgare: | | | |
| (XP_044984305.1) | | | |
| Triticum_aestivum: | 79.378 | 86.7 | |
| (UniProt: | | | |
| A0A3B5Z6B7) | | | |
| Aegilops tauschii | 80.524 | 88.177 | 97.039 |
| subsp. | | | |
| tauschii: (RefSeq: | | | |
| XP_020178692.1) | | | |

SEQUENCE ALIGNMENT 3
SPO11-1 protein sequences from dicotyledonous plants.
SPO11-1 Dicots
Homologs identified by BLAST of Arabidopsis thaliana [symbol] map to
dicot NCBI RefSeq, UniProtKB, Vaccinium [symbol] Draper v1.0, [symbol]
v3.0, Citrullus lanatus (97103) v2.0, and Citrullus lanatus
(Charleston Gray) v1.0 protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Citrullus lanatus: (Cla [symbol] )* [symbol] | [symbol] |
| Rubus occidentalis: [symbol] | [symbol] |
| Vaccinium corymbosum: ( [symbol] ) | [symbol] |
| Arabidopsis thaliana: (UniProt: [symbol] ) | [symbol] |
| Gossypium barbadense: (UniProt: [symbol] ) | [symbol] |
| Prunus avium: (UniProt: [symbol] ) | [symbol] |
| Solanum tuberosum: (RefSeq:XP_ [symbol] ) | [symbol] |
| Solanum lycopersicum:(RefSeq:XP_ [symbol] ) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Citrullus lanatus: (Cla [symbol] )* [symbol] | [symbol] |
| Rubus occidentalis: [symbol] | [symbol] |
| Vaccinium corymbosum: ( [symbol] ) | [symbol] |
| Arabidopsis thaliana: (UniProt: [symbol] ) | [symbol] |
| Gossypium barbadense: (UniProt: [symbol] ) | [symbol] |
| Prunus avium: (UniProt: [symbol] ) | [symbol] |
| Solanum tuberosum: (RefSeq:XP_ [symbol] ) | [symbol] |
| Solanum lycopersicum:(RefSeq:XP_ [symbol] ) | [symbol] |
| | 210 220 230 240 250 260 270 280 290 300 |
| | ----:----\|----:----\|----:----\|----:----\|----:----\|----:----\|----:----\|----:----\|----:----\|----:----\| |
| Consensus | [symbol] |
| Citrullus lanatus: (Cla [symbol] )* [symbol] | [symbol] |
| Rubus occidentalis: [symbol] | [symbol] |
| Vaccinium corymbosum: ( [symbol] ) | [symbol] |
| Arabidopsis thaliana: (UniProt: [symbol] ) | [symbol] |
| Gossypium barbadense: (UniProt: [symbol] ) | [symbol] |
| Prunus avium: (UniProt: [symbol] ) | [symbol] |
| Solanum tuberosum: (RefSeq:XP_ [symbol] ) | [symbol] |
| Solanum lycopersicum:(RefSeq:XP_ [symbol] ) | [symbol] |
| | 310 320 330 340 350 360 370 380 390 400 |
| | ----:----\|----:----\|----:----\|----:----\|----:----\|----:----\|----:----\|----:----\|----:----\|----:----\| |
| Consensus | [symbol] |
| Citrullus lanatus: (Cla [symbol] )* [symbol] | [symbol] |
| Rubus occidentalis: [symbol] | [symbol] |
| Vaccinium corymbosum: ( [symbol] ) | [symbol] |
| Arabidopsis thaliana: (UniProt: [symbol] ) | [symbol] |
| Gossypium barbadense: (UniProt: [symbol] ) | [symbol] |
| Prunus avium: (UniProt: [symbol] ) | [symbol] |
| Solanum tuberosum: (RefSeq:XP_ [symbol] ) | [symbol] |
| Solanum lycopersicum:(RefSeq:XP_ [symbol] ) | [symbol] |
| | 410 420 430 440 450 460 470 480 490 500 |
| | ----:----\|----:----\|----:----\|----:----\|----:----\|----:----\|----:----\|----:----\|----:----\|----:----\| |
| Consensus | [symbol] |
| Citrullus lanatus: (Cla [symbol] )* [symbol] | [symbol] |
| Rubus occidentalis: [symbol] | [symbol] |
| Vaccinium corymbosum: ( [symbol] ) | [symbol] |
| Arabidopsis thaliana: (UniProt: [symbol] ) | [symbol] |
| Gossypium barbadense: (UniProt: [symbol] ) | [symbol] |
| Prunus avium: (UniProt: [symbol] ) | [symbol] |
| Solanum tuberosum: (RefSeq:XP_ [symbol] ) | [symbol] |
| Solanum lycopersicum:(RefSeq:XP_ [symbol] ) | [symbol] |
| | 510 520 530 540 550 560 570 |
| | ----:----\|----:----\|----:----\|----:----\|----:----\|----:----\|----:----\|----:--- |
| Consensus | [symbol] |
| Citrullus lanatus: (Cla [symbol] )* [symbol] | ------------------------------------------------------------------------------ |
| Rubus occidentalis: [symbol] | ------------------------------------------------------------------------------ |
| Vaccinium corymbosum: ( [symbol] ) | .....S........................................................................ |
| Arabidopsis thaliana: (UniProt: [symbol] ) | ------------------------------------------------------------------------------ |
| Gossypium barbadense: (UniProt: [symbol] ) | ------------------------------------------------------------------------------ |
| Prunus avium: (UniProt: [symbol] ) | ------------------------------------------------------------------------------ |
| Solanum tuberosum: (RefSeq:XP_ [symbol] ) | ------------------------------------------------------------------------------ |
| Solanum lycopersicum:(RefSeq:XP_ [symbol] ) | -----N-----------------------------------------------------------------------. |

[symbol]

indicates data missing or illegible when filed

TABLE 3
Percent identities of SPO11-1 protein sequences from dicotyledonous plants.

| | | | Vaccinium | | | | Solanum |
|---|---|---|---|---|---|---|---|
| | Citrullus | Rubus | corymbosum: | Arabidopsis | Gossypium | Prunus | tuberosum: |
| | lanatus: | occidentalis: | (VaccDscaff83- | thaliana: | barbadense: | avium: | (RefSeq: |
| SPO11-1 | (Cla97C04G075440.1)* | (Ro01G11692)* | snap-gene-5.35- | (UniProt: | (UniProt: | (UniProt: | XP_{—} |
| Dicot % ID | ClCG09G021210.1 | Ro03_G32130 | mRNA-1) | Q9M4A2) | A0A5J5SLP4) | A0A6P5TTD7) | 006346146.1) |
| Rubus | 46.985 | | | | | | |
| occidentalis: | | | | | | | |
| (Ro01G11692)* | | | | | | | |
| Ro03_G32130 | | | | | | | |
| Vaccinium | 35.599 | 37.678 | | | | | |
| corymbosum: | | | | | | | |
| (VaccDscaff8 | | | | | | | |
| 3-snap-gene- | | | | | | | |
| 5.35-mRNA-1) | | | | | | | |
| Arabidopsis | 49.353 | 59.424 | 43.396 | | | | |
| thaliana: | | | | | | | |
| (UniProt: | | | | | | | |
| Q9M4A2) | | | | | | | |
| Gossypium | 45.474 | 57.333 | 36.134 | 59.669 | | | |
| barbadense: | | | | | | | |
| (UniProt: | | | | | | | |
| A0A5J5SLP4) | | | | | | | |
| Prunus | 52.778 | 74.278 | 43.659 | 68.579 | 61.37 | | |
| avium: | | | | | | | |
| (UniProt: | | | | | | | |
| A0A6P5TTD7) | | | | | | | |
| Solanum | 50.647 | 64.921 | 42.977 | 66.851 | 59.392 | 71.585 | |
| tuberosum: | | | | | | | |
| (RefSeq: | | | | | | | |
| XP_006346146.1) | | | | | | | |
| Solanum | 48.861 | 61.042 | 44.025 | 63.708 | 56.919 | 67.959 | 90.601 |
| lycopersicum: | | | | | | | |
| (RefSeq: | | | | | | | |
| XP_010324270.1) | | | | | | | |

SEQUENCE ALIGNMENT 4
SPO11-1 sequences from monocotyledonous plants.
SPO11-1 Monocots
Homologs identified by BLAST of Oryza sativa Os03g54091.1 from MSU-OSA1 Release
7 map to monocot NCBI RefSeq, GenBank and UniProtKB protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | MNAGRWIDIXKRRRATVLLAAFVPSFXDDDERRGRRRLEEAALSDLLHXIKGLVXWVVAEVSAGRSPSXALHRYQNYCAXADSAAAAASPSTCACSYDAP |
| Aegilops tauschii:(RefSeq:XP_020158624.1) | .-..S----G........---------................--...K.....G.....I.......L..........S..A................. |
| Brachypodium_distachyon:(RefSeq:XP_003559449.1) | .-E..----E........--------D....L.....EQ....--...K.....G......G......V..N......-----TS.....P......... |
| Hordeum_vulgare_subsp._vulgare:(UniProt: [symbol] ) | .-...----G........---------.............S..--...K.....G.....L.......L..........---.................. |
| Musa_acuminata_subsp._malaccensis:(XP_009380133.2) | ..V......DAQA.PIF.........AQATT.MAGG.VSSSSP....QR...F.RSL.EDL.ND.P..V..D..R...--------HDPSGN.T.GDNL. |
| Oryza_sativa_Japonica_Group: | .-...----E....VAA.---------.GE..--...Q....T--...R.R...R......A.....TV.........----.S.S..AA.P......V. |
| (RefSeq:NP_001391702.1) | |
| Sorghum_bicolor:(RefSeq:XP_002466440.1) | .-...----D.....AP.---------EG..QQL...Q.....--..RR.....R...E..A......IV....R...----...DS....P......V. |
| Triticum_aestivum:(UniProt:A0A3B6LTD1) | .-..S----G........---------................--...K.....G.....I.......L..........C.-.................. |
| Zea_mays:(RefSeq:NP_001347894.1) | .-...----D.....AP.---------EG..QQL.........--..RR.....R.I.E..A......IV....R...----.S.DS....P......I. |
| | 110 120 130 140 150 160 170 180 190 200 |
| | ----:----\|----:----\|----:----\|----:----\|----:----\|----:----\|----:----\|----:----\|----:----\|----:----\| |
| Consensus | VGTDVLSLLRKEFHASRLNVLLRVLLVVQQLLQZNKHCSKRDIYYMYPSIFVEQAVVDRAINDICILFKCSRHNLNVVPVAKGLVMGWIRFVEGEKKVYC |
| Aegilops tauschii:(RefSeq:XP_020158624.1) | .................................E...............M.....I............................................ |
| Brachypodium_distachyon:(RefSeq:XP_003559449.1) | ....I.C...........S......F.......Q.................I................................................ |
| Hordeum_vulgare_subsp._vulgare:(UniProt: [symbol] ) | .................................E...............M.....I............................................ |
| Musa_acuminata_subsp._malaccensis:(XP_009380133.2) | N.KEII.-VER.S..Y..L..F....II.....E...G........H.AL.L..........N....L....QH......GR......L..L.AGR.I.. |
| Oryza_sativa_Japonica_Group: | .........HRGS....................Q.................Q.............V.........................L....E... |
| (RefSeq:NP_001391702.1) | |
| Sorghum_bicolor:(RefSeq:XP_002466440.1) | .........HNDY.T..............K...Q...................V.............L............V................... |
| Triticum_aestivum:(UniProt:A0A3B6LTD1) | .................................E...............M.....I............................................ |
| Zea_mays:(RefSeq:NP_001347894.1) | .........H.DY.T..........F.......Q...................V..........................V..........M........ |
| | 210 220 230 240 250 260 270 280 290 300 |
| | ----:----\|----:----\|----:----\|----:----\|----:----\|----:----\|----:----\|----:----\|----:----\|----:----\| |
| Consensus | ITNVNAAFXIPVSIEAIKDVVSVAHYILVVEKETVFQRLANDKFCERNRCIVITGRGYPDIPTRRFLRYLVEQLHLPAYCLVDSDPYGFDILATYKFGSM |
| Aegilops tauschii:(RefSeq:XP_020158624.1) | ........P........................A............K..................................................... |
| Brachypodium_distachyon:(RefSeq:XP_003559449.1) | ........S......T...I........................................V.......H............................... |
| Hordeum_vulgare_subsp._vulgare:(UniProt: [symbol] ) | .....T..P........................A...........KK...........................R......................... |
| Musa_acuminata_subsp._malaccensis:(XP_009380133.2) | .NSPSTVYP...CL.EVV.I....RF.I.....S........R.....H...........V......CL..QH....V.....C........MV.R.... |
| Oryza_sativa_Japonica_Group: | V.......S...............D...I................................................V.....A...............L |
| (RefSeq:NP_001391702.1) | |
| Sorghum_bicolor:(RefSeq:XP_002466440.1) | ........S...D......................................................................................L |
| Triticum_aestivum:(UniProt:A0A3B6LTD1) | .....T..P........................A............K..................................................... |
| Zea_mays:(RefSeq:NP_001347894.1) | ..S.....S...D...........................................................L..........................L |
| | 310 320 330 340 350 360 370 380 390 400 |
| | ----:----\|----:----\|----:----\|----:----\|----:----\|----:----\|----:----\|----:----\|----:----\|----:----\| |
| Consensus | QLAYDANLLRVPEIRWLGVFTSDFEEYCLPDCCLLXLSSEDRRKXEGILTRCYLHREAPEWRLELEAMLZKGVKFEIEALSASSISFLSQEYIPQKIKLG |
| Aegilops tauschii:(RefSeq:XP_020158624.1) | .......V.........................Q.Q........L..........K.......K.....EM........................Q.R.. |
| Brachypodium_distachyon:(RefSeq:XP_003559449.1) | ............G..................Y...D......K.L.................A......E.............................. |
| Hordeum_vulgare_subsp._vulgare:(UniProt: [symbol] ) | .M.....V.........................Q.P.....T..L..................K..E..E...........S.................. |
| Musa_acuminata_subsp._malaccensis:(XP_009380133.2) | .M............S.....H..LQK.H...R...E.T.D.KK.A.AM.L....QK...K..M...V..QT.......S..FN.L....-....T..HD. |
| Oryza_sativa_Japonica_Group: | .......F....D............D.R.......H........A....S.........Q.........Q............C......E....K...Q. |
| (RefSeq:NP_001391702.1) | |
| Sorghum_bicolor:(RefSeq:XP_002466440.1) | ...H........D......................H..P.....A.R..A.......V....S......Q............N......D........Q. |
| Triticum_aestivum:(UniProt:A0A3B6LTD1) | .......I.........................Q.Q........L..........K.......K.....EM........................Q.R.. |
| Zea_mays:(RefSeq:NP_001347894.1) | ...H........D......................R..P.....A....A............S......Q............N......H........Q. |
| | --- |
| Consensus | RXI |
| Aegilops tauschii:(RefSeq:XP_020158624.1) | .Y. |
| Brachypodium_distachyon:(RefSeq:XP_003559449.1) | .H. |
| Hordeum_vulgare_subsp._vulgare:(UniProt: [symbol] ) | .Y. |
| Musa_acuminata_subsp._malaccensis:(XP_009380133.2) | SYL |
| Oryza_sativa_Japonica_Group: | .H. |
| (RefSeq:NP_001391702.1) | |
| Sorghum_bicolor:(RefSeq:XP_002466440.1) | MHL |
| Triticum_aestivum:(UniProt:A0A3B6LTD1) | .V. |
| Zea_mays:(RefSeq:NP_001347894.1) | MHL |

[symbol]

indicates data missing or illegible when filed

TABLE 4
Percent identities of SPO11-1 protein sequences from monocotyledonous plants.

| | | | Sorghum | Oryza sativa |
|---|---|---|---|---|
| | Musa acuminata | Zea mays: | bicolor: | Japonic Group: |
| SPO11-1 | subsp. malaccensis: | (RefSeq: | (RefSeq: | (RefSeq: |
| Monocot % ID | (XP_009380133.2) | XP_008643458.1) | XP_021304292.1) | XP_015630525.1) |
| Zea mays: | 42.384 | | | |
| (RefSeq: | | | | |
| XP_008643458.1) | | | | |
| Sorghum | 48.25 | 80.867 | | |
| bicolor: | | | | |
| (RefSeq: | | | | |
| XP_021304292.1) | | | | |
| Oryza sativa | 50.877 | 56.522 | 65.274 | |
| Japonic Group: | | | | |
| (RefSeq: | | | | |
| XP_015630525.1) | | | | |
| Aegilops | 44.717 | 49.533 | 54.948 | 53.671 |
| tauschii: | | | | |
| (UniProt: | | | | |
| M8BE82) | | | | |
| Brachypodium | 57.895 | 62.557 | 71.354 | 73.958 |
| distachyon: | | | | |
| (RefSeq: | | | | |
| XP_003559449.1) | | | | |
| Triticum | 57.463 | 61.327 | 69.43 | 74.352 |
| aestivum: | | | | |
| (UniProt: | | | | |
| A0A3B6LTD1) | | | | |
| Hordeum | 58 | 61.327 | 69.531 | 73.698 |
| vulgare subsp. | | | | |
| vulgare: | | | | |
| (UniProt: | | | | |
| A0A287SD60) | | | | |

| | Aegilops | Brachypodium | Triticum |
|---|---|---|---|
| | tauschii: | distachyon: | aestivum: |
| SPO11-1 | (UniProt: | (RefSeq: | (UniProt: |
| Monocot % ID | M8BE82) | XP_003559449.1) | A0A3B6LTD1) |
| Zea mays: | | | |
| (RefSeq: | | | |
| XP_008643458.1) | | | |
| Sorghum | | | |
| bicolor: | | | |
| (RefSeq: | | | |
| XP_021304292.1) | | | |
| Oryza sativa | | | |
| Japonic Group: | | | |
| (RefSeq: | | | |
| XP_015630525.1) | | | |
| Aegilops | | | |
| tauschii: | | | |
| (UniProt: | | | |
| M8BE82) | | | |
| Brachypodium | 66.076 | | |
| distachyon: | | | |
| (RefSeq: | | | |
| XP_003559449.1) | | | |
| Triticum | 72.362 | 86.563 | |
| aestivum: | | | |
| (UniProt: | | | |
| A0A3B6LTD1) | | | |
| Hordeum | 69.949 | 87.013 | 94.301 |
| vulgare subsp. | | | |
| vulgare: | | | |
| (UniProt: | | | |
| A0A287SD60) | | | |

SEQUENCE ALIGNMENT 5
PAIR1 protein sequences from dicotyledonous plants
PAIR1 Dicots
Homologs identified by BLAST of Arabidopsis thaliana [symbol] -TAIR10 map to dicot NCBI RefSeq,
UniProtKB, Vaccinium corymbosum Draper v1.0, Rubus occidentalis v3.0,
Citrullus lanatus (97103) v2.0, and Citrullus lanatus (Charleston Gray) v1.0 protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Arabidopsis_thaliana:(RefSeq:NP_ [symbol] ) | [symbol] |
| Citrullus_lanatus:_(Cla [symbol] ) | [symbol] |
| Solanum_tuberosum:(RefSeq:XP_ [symbol] ) | [symbol] |
| Solanum_lycopersicum:(RefSeq:XP_ [symbol] ) | [symbol] |
| Vaccinium_corymbosum:_(VaccDscaff40- [symbol] ) | [symbol] |
| Gossypium_barbadense:(UniProt: [symbol] ) | [symbol] |
| Prunus_avium:(UniProt: [symbol] ) | [symbol] |
| Rubus_occidentalis:_( [symbol] ) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Arabidopsis_thaliana:(RefSeq:NP_ [symbol] ) | [symbol] |
| Citrullus_lanatus:_(Cla [symbol] ) | [symbol] |
| Solanum_tuberosum:(RefSeq:XP_ [symbol] ) | [symbol] |
| Solanum_lycopersicum:(RefSeq:XP_ [symbol] ) | [symbol] |
| Vaccinium_corymbosum:_(VaccDscaff40- [symbol] ) | [symbol] |
| Gossypium_barbadense:(UniProt: [symbol] ) | [symbol] |
| Prunus_avium:(UniProt: [symbol] ) | [symbol] |
| Rubus_occidentalis:_( [symbol] ) | [symbol] |
| | 210 220 230 240 250 260 270 280 290 300 |
| | ----:----\|----:----\|----:----\|----:----\|----:----\|----:----\|----:----\|----:----\|----:----\|----:----\| |
| Consensus | [symbol] |
| Arabidopsis_thaliana:(RefSeq:NP_ [symbol] ) | [symbol] |
| Citrullus_lanatus:_(Cla [symbol] ) | [symbol] |
| Solanum_tuberosum:(RefSeq:XP_ [symbol] ) | [symbol] |
| Solanum_lycopersicum:(RefSeq:XP_ [symbol] ) | [symbol] |
| Vaccinium_corymbosum:_(VaccDscaff40- [symbol] ) | [symbol] |
| Gossypium_barbadense:(UniProt: [symbol] ) | [symbol] |
| Prunus_avium:(UniProt: [symbol] ) | [symbol] |
| Rubus_occidentalis:_( [symbol] ) | [symbol] |
| | 310 320 330 340 350 360 370 380 390 400 |
| | ----:----\|----:----\|----:----\|----:----\|----:----\|----:----\|----:----\|----:----\|----:----\|----:----\| |
| Consensus | [symbol] |
| Arabidopsis_thaliana:(RefSeq:NP_ [symbol] ) | [symbol] |
| Citrullus_lanatus:_(Cla [symbol] ) | [symbol] |
| Solanum_tuberosum:(RefSeq:XP_ [symbol] ) | [symbol] |
| Solanum_lycopersicum:(RefSeq:XP_ [symbol] ) | [symbol] |
| Vaccinium_corymbosum:_(VaccDscaff40- [symbol] ) | [symbol] |
| Gossypium_barbadense:(UniProt: [symbol] ) | [symbol] |
| Prunus_avium:(UniProt: [symbol] ) | [symbol] |
| Rubus_occidentalis:_( [symbol] ) | [symbol] |
| | 410 420 430 440 450 460 470 480 490 500 |
| | ----:----\|----:----\|----:----\|----:----\|----:----\|----:----\|----:----\|----:----\|----:----\|----:----\| |
| Consensus | [symbol] |
| Arabidopsis_thaliana:(RefSeq:NP_ [symbol] ) | [symbol] |
| Citrullus_lanatus:_(Cla [symbol] ) | [symbol] |
| Solanum_tuberosum:(RefSeq:XP_ [symbol] ) | [symbol] |
| Solanum_lycopersicum:(RefSeq:XP_ [symbol] ) | [symbol] |
| Vaccinium_corymbosum:_(VaccDscaff40- [symbol] ) | [symbol] |
| Gossypium_barbadense:(UniProt: [symbol] ) | [symbol] |
| Prunus_avium:(UniProt: [symbol] ) | [symbol] |
| Rubus_occidentalis:_( [symbol] ) | [symbol] |

[symbol]

indicates data missing or illegible when filed

TABLE 5
Percent identities of PAIR1 protein sequences from dicotyledonous plants.

| | Arabidopsis | | Solanum | Solanum |
|---|---|---|---|---|
| | thaliana: | | tuberosum: | lycopersicum: |
| PAIR1 Dicot % | (Ref Seq: | Citrullus lanatus: | (RefSeq: | (RefSeq: |
| ID | NP_001318904.1) | (Cla97C05G081880.1) | XP_006339791.2) | XP_004231186.2) |
| Citrullus | 35.205 | | | |
| lanatus: | | | | |
| (Cla97C05G081880.1) | | | | |
| Solanum | 35.381 | 34.348 | | |
| tuberosum: | | | | |
| (RefSeq: | | | | |
| XP_006339791.2) | | | | |
| Solanum | 35.821 | 34.658 | 90.466 | |
| lycopersicum: | | | | |
| (RefSeq: | | | | |
| XP_004231186.2) | | | | |
| Vaccinium | 36.105 | 40.98 | 40.249 | 41.263 |
| corymbosum: | | | | |
| (VaccDscaff40- | | | | |
| augustus-gene- | | | | |
| 183.9-mRNA-1) | | | | |
| Gossypium | 37.961 | 45.631 | 40.611 | 40.889 |
| barbadense: | | | | |
| (UniProt: | | | | |
| A0A5J5QG99) | | | | |
| Prunus | 38.261 | 47.722 | 40.652 | 41.333 |
| avium: (UniProt: | | | | |
| A0A6P5RCR3) | | | | |
| Rubus | 41.379 | 37.609 | 40.302 | 39.827 |
| occidentalis: | | | | |
| (Ro07_G04309) | | | | |

| | Vaccinium | | |
|---|---|---|---|
| | corymbosum: | | |
| | (VaccDscaff40- | Gossypium | Prunus |
| | augustus- | barbadense: | avium: |
| PAIR1 Dicot % | gene-183.9- | (UniProt: | (UniProt: |
| ID | mRNA-1) | A0A5J5QG99) | A0A6P5RCR3) |
| Citrullus | | | |
| lanatus: | | | |
| (Cla97C05G081880.1) | | | |
| Solanum | | | |
| tuberosum: | | | |
| (RefSeq: | | | |
| XP_006339791.2) | | | |
| Solanum | | | |
| lycopersicum: | | | |
| (RefSeq: | | | |
| XP_004231186.2) | | | |
| Vaccinium | | | |
| corymbosum: | | | |
| (VaccDscaff40- | | | |
| augustus-gene- | | | |
| 183.9-mRNA-1) | | | |
| Gossypium | 51.007 | | |
| barbadense: | | | |
| (UniProt: | | | |
| A0A5J5QG99) | | | |
| Prunus | 51.333 | 55.474 | |
| avium: (UniProt: | | | |
| A0A6P5RCR3) | | | |
| Rubus | 41.309 | 44.079 | 57.86 |
| occidentalis: | | | |
| (Ro07_G04309) | | | |

SEQUENCE ALIGNMENT 6
PAIR1 protein sequences from monocotyledonous plants.
PAIR1 Monocots
Homologs identified by BLAST of Oryza sativa [symbol] from MSU - Osa1 Release
7 map to monocot NCBI RefSeq, GenBank and UniProtKB protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 210 220 230 240 250 260 270 280 290 300 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 310 320 330 340 350 360 370 380 390 400 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 410 420 430 440 450 460 470 480 490 500 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 510 520 530 540 550 560 570 580 590 600 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 610 620 630 640 650 660 670 680 690 700 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 710 720 730 740 750 760 770 780 790 800 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |
| | 810 820 830 840 850 860 870 |
| | ----:----\|----:----\|----:----\|----:----\|----:----\|----:----\|----:----\|-- |
| Consensus | [symbol] |
| Musa_acuminata_subsp._malaccensis:(RefSeq:XP_ [symbol] ) | [symbol] |
| Triticum_aestivum:(UniProt: [symbol] ) | [symbol] |
| Hordeum_vulgare_subsp._vulgare:(RefSeq:XP_ [symbol] ) | [symbol] |
| Aegilops_tauschii:(UniProt: [symbol] ) | [symbol] |
| Brachypodium_distachyon:(RefSeq:XP_ [symbol] ) | [symbol] |
| Sorghum_bicolor:(UniProt: [symbol] ) | [symbol] |
| Zea_mays:(RefSeq:XP_ [symbol] ) | [symbol] |
| Zea_mays:(GenBank: [symbol] ) | [symbol] |
| Oryza_sativa_Japonica_Group:(RefSeq:XP_ [symbol] ) | [symbol] |

[symbol]

indicates data missing or illegible when filed

TABLE 6
Percent identities of PAIR1 protein sequences from monocotyledonous plants.

| | Musa | | | | |
|---|---|---|---|---|---|
| | acuminata | | Hordeum | | |
| | subsp. | Triticum | vulgare subsp. | Aegilops | Brachypodium |
| | malaccensis: | aestivum: | vulgare: | tauschii: | distachyon: |
| PAIR1 | (RefSeq: | (UniProt: | (RefSeq: | (UniProt: | (RefSeq: |
| Monocot % ID | XP_018685297.1) | A0A3B6C7X9) | XP_044983266.1) | N1R2L9) | XP_014751501.1) |
| Triticum | 23.184 | | | | |
| aestivum: | | | | | |
| (UniProt: | | | | | |
| A0A3B6C7X9) | | | | | |
| Hordeum | 22.141 | 57.324 | | | |
| vulgare subsp. | | | | | |
| vulgare: | | | | | |
| (RefSeq: | | | | | |
| XP_044983266.1) | | | | | |
| Aegilops | 18.502 | 25.701 | 22.895 | | |
| tauschii: | | | | | |
| (UniProt: | | | | | |
| N1R2L9) | | | | | |
| Brachypodium | 17.04 | 30.976 | 28.61 | 29.61 | |
| distachyon: | | | | | |
| (RefSeq: | | | | | |
| XP_014751501.1) | | | | | |
| Sorghum | 22.687 | 38.108 | 35.525 | 24.434 | 29.184 |
| bicolor: | | | | | |
| (UniProt: | | | | | |
| A0A1B6QR58) | | | | | |
| Zea mays: | 27.083 | 42.178 | 39.08 | 27.098 | 31.013 |
| (RefSeq: | | | | | |
| XP_008660580.1) | | | | | |
| Zea mays: | 24.598 | 38.937 | 36.156 | 24.959 | 29.602 |
| (GenBank: | | | | | |
| ONL92656.1) | | | | | |
| Oryza sativa | 29.354 | 38.735 | 36.75 | 27.006 | 32.721 |
| Japonica | | | | | |
| Group: | | | | | |
| (RefSeq: | | | | | |
| XP_015632198.1) | | | | | |

| | Sorghum | | |
|---|---|---|---|
| | bicolor: | Zea mays: | Zea mays: |
| PAIR1 | (UniProt: | (RefSeq: | (GenBank: |
| Monocot % ID | A0A1B6QR58) | XP_008660580.1) | ONL92656.1) |
| Triticum | | | |
| aestivum: | | | |
| (UniProt: | | | |
| A0A3B6C7X9) | | | |
| Hordeum | | | |
| vulgare subsp. | | | |
| vulgare: | | | |
| (RefSeq: | | | |
| XP_044983266.1) | | | |
| Aegilops | | | |
| tauschii: | | | |
| (UniProt: | | | |
| N1R2L9) | | | |
| Brachypodium | | | |
| distachyon: | | | |
| (RefSeq: | | | |
| XP_014751501.1) | | | |
| Sorghum | | | |
| bicolor: | | | |
| (UniProt: | | | |
| A0A1B6QR58) | | | |
| Zea mays: | 66.269 | | |
| (RefSeq: | | | |
| XP_008660580.1) | | | |
| Zea mays: | 69.4 | 67.903 | |
| (GenBank: | | | |
| ONL92656.1) | | | |
| Oryza sativa | 39.493 | 44.658 | 42.165 |
| Japonica | | | |
| Group: | | | |
| (RefSeq: | | | |
| XP_015632198.1) | | | |

SEQUENCE ALIGNMENT 7
OSD1 sequences from dicotyledonous plants.
OSD1 Dicots
Homologs identified by BLAST of Arabidopsis thaliana [symbol]
map to dicot NCBI RefSeq, UniProtKB, Vaccinium [symbol] Draper v1.0,
Rubus occidentalis v3.0, Citrullus lanatus (97103) v2.0, and
Citrullus lanatus (Charleston Gray) v1.0 protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Prunus avium: (RefSeq:XP_ [symbol] ) | [symbol] |
| Rubus occidentalils: ( [symbol] ) | [symbol] |
| Arabidopsis thaliana: (UniProt: [symbol] ) | [symbol] |
| Gossypium barbadense: (UniProt: [symbol] ) | [symbol] |
| Citrullus lanatus: (Cla [symbol] ) | [symbol] |
| Vaccinium corymbosum: ( [symbol] ) | [symbol] |
| Solanum lycopersicum: (UniProt: [symbol] ) | [symbol] |
| Solanum tuberosum: (RefSeq:XP_ [symbol] ) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Prunus avium: (RefSeq:XP_ [symbol] ) | [symbol] |
| Rubus occidentalils: ( [symbol] ) | [symbol] |
| Arabidopsis thaliana: (UniProt: [symbol] ) | [symbol] |
| Gossypium barbadense: (UniProt: [symbol] ) | [symbol] |
| Citrullus lanatus: (Cla [symbol] ) | [symbol] |
| Vaccinium corymbosum: ( [symbol] ) | [symbol] |
| Solanum lycopersicum: (UniProt: [symbol] ) | [symbol] |
| Solanum tuberosum: (RefSeq:XP_ [symbol] ) | [symbol] |
| | 210 220 230 240 250 260 270 |
| | ----:----\|----:----\|----:----\|----:----\|----:----\|----:----\|----:----\|----:--- |
| Consensus | [symbol] |
| Prunus avium: (RefSeq:XP_ [symbol] ) | [symbol] |
| Rubus occidentalils: ( [symbol] ) | [symbol] |
| Arabidopsis thaliana: (UniProt: [symbol] ) | [symbol] |
| Gossypium barbadense: (UniProt: [symbol] ) | [symbol] |
| Citrullus lanatus: (Cla [symbol] ) | [symbol] |
| Vaccinium corymbosum: ( [symbol] ) | [symbol] |
| Solanum lycopersicum: (UniProt: [symbol] ) | [symbol] |
| Solanum tuberosum: (RefSeq:XP_ [symbol] ) | [symbol] |

[symbol]

indicates data missing or illegible when filed

TABLE 7A
Percent identities of OSD1 protein sequences from dicotyledonous plants.

| | | | Arabidopsis | Gossypium |
|---|---|---|---|---|
| OSD1 | Prunus avium: | Rubus | thaliana: | barbadense: |
| Dicot % | (RefSeq: | occidentalis: | (UniProt: | (UniProt: |
| ID | XP_021833341.1) | (Ro06G14226) | Q9M2R1) | A0A2P5XW92) |
| Rubus | 45.588 | | | |
| occidentalis: | | | | |
| (Ro06 G14226) | | | | |
| Arabidopsis | 41.887 | 37.778 | | |
| thaliana: | | | | |
| (UniProt: Q9M2R1) | | | | |
| Gossypium | 46.442 | 41.455 | 40.377 | |
| barbadense: | | | | |
| (UniProt: | | | | |
| A0A2P5XW92) | | | | |
| Citrullus | 45.865 | 40.959 | 40.909 | 45.113 |
| lanatus: | | | | |
| (Cla97C04G068720.1) | | | | |
| Vaccinium | 47.17 | 41.176 | 41.154 | 49.434 |
| corymbosum: | | | | |
| (VaccDscaff5- | | | | |
| snap-gene- | | | | |
| 277.41-mRNA-1) | | | | |
| Solanum | 44.697 | 38.889 | 40.698 | 47.348 |
| lycopersicum: | | | | |
| UniProt: | | | | |
| (A0A3Q7ILG3) | | | | |
| Solanum | 44.697 | 38.148 | 40.698 | 46.591 |
| tuberosum: | | | | |
| (RefSeq: | | | | |
| XP_006351336.1) | | | | |

| | | Vaccinium | |
|---|---|---|---|
| | | corymbosum: | Solanum |
| OSD1 | Citrullus | (VaccDscaff5- | lycopersicum: |
| Dicot % | lanatus: | snap-gene- | (UniProt: |
| ID | (Cla97C04G068720.1) | 277.41-mRNA-1) | A0A3Q7ILG3) |
| Rubus | | | |
| occidentalis: | | | |
| (Ro06 G14226) | | | |
| Arabidopsis | | | |
| thaliana: | | | |
| (UniProt: Q9M2R1) | | | |
| Gossypium | | | |
| barbadense: | | | |
| (UniProt: | | | |
| A0A2P5XW92) | | | |
| Citrullus | | | |
| lanatus: | | | |
| (Cla97C04G068720.1) | | | |
| Vaccinium | 47.909 | | |
| corymbosum: | | | |
| (VaccDscaff5- | | | |
| snap-gene- | | | |
| 277.41-mRNA-1) | | | |
| Solanum | 49.042 | 49.615 | |
| lycopersicum: | | | |
| UniProt: | | | |
| (A0A3Q7ILG3) | | | |
| Solanum | 48.276 | 49.615 | 96.471 |
| tuberosum: | | | |
| (RefSeq: | | | |
| XP_006351336.1) | | | |

SEQUENCE ALIGNMENT 8
OSD1 protein sequences from monocotyledonous plants.
OSD1 Monocots
Homologs identified by BLAST of Oryza sativa Os02g3785.1 from MSU-Osa1
Release 7 map to monocot NCBI RefSeq, GenBank and UniProtKB protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis: | [symbol] |
| (RefSeq: XP_009415567.1) | |
| Brachypodium distachyon: (UniProt: [symbol] ) | [symbol] |
| Hordeum vulgare subsp. vulgare: (UniProt: F2EGV1) | [symbol] |
| Triticum aestivum: (UniProt: [symbol] ) | [symbol] |
| Aegilops tauschii: (UniProt: M8CG24) | [symbol] |
| Sorghum bicolor: (UniProt: C5XWE8) | [symbol] |
| Oryza sativa: (RefSeq: XP_015635088) | [symbol] |
| Zea mays: (UniProt: 84FG75) | [symbol] |
| Zea mays: (GenBank: AQK72186.1) | [symbol] |
| Zea mays: (GenBank: AQK72188.1) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Musa_acuminata_subsp._malaccensis: | [symbol] |
| (RefSeq: XP_009415567.1) | |
| Brachypodium distachyon: (UniProt: [symbol] ) | [symbol] |
| Hordeum vulgare subsp. vulgare: (UniProt: F2EGV1) | [symbol] |
| Triticum aestivum: (UniProt: [symbol] ) | [symbol] |
| Aegilops tauschii: (UniProt: M8CG24) | [symbol] |
| Sorghum bicolor: (UniProt: C5XWE8) | [symbol] |
| Oryza sativa: (RefSeq: XP_015635088) | [symbol] |
| Zea mays: (UniProt: 84FG75) | [symbol] |
| Zea mays: (GenBank: AQK72186.1) | [symbol] |
| Zea mays: (GenBank: AQK72188.1) | [symbol] |
| | 210 220 230 240 250 260 |
| | ----:----\|----:----\|----:----\|----:----\|----:----\|----:----\|----: |
| Consensus | [symbol] |
| Musa_acuminata_subsp._malaccensis: | [symbol] |
| (RefSeq: XP_009415567.1) | |
| Brachypodium distachyon: (UniProt: [symbol] ) | [symbol] |
| Hordeum vulgare subsp. vulgare: (UniProt: F2EGV1) | [symbol] |
| Triticum aestivum: (UniProt: [symbol] ) | [symbol] |
| Aegilops tauschii: (UniProt: M8CG24) | [symbol] |
| Sorghum bicolor: (UniProt: C5XWE8) | [symbol] |
| Oryza sativa: (RefSeq: XP_015635088) | [symbol] |
| Zea mays: (UniProt: 84FG75) | [symbol] |
| Zea mays: (GenBank: AQK72186.1) | [symbol] |
| Zea mays: (GenBank: AQK72188.1) | [symbol] |

[symbol]

indicates data missing or illegible when filed

TABLE 7B
Percent identities of OSD1 sequences from monocotyledonous plants.

| | Musa | | Hordeum | | |
|---|---|---|---|---|---|
| | acuminata | | vulgare | | |
| | subsp. | Brachypodium | subsp. | Triticum | Aegilops |
| OSD1 | malaccensis: | distachyon: | vulgare: | aestivum: | tauschii: |
| Monocot % | (RefSeq: | (UniProt: | (UniProt: | (UniProt: | (UniProt: |
| ID | XP_009415567.1) | I1IB35) | F2EGV1) | A0A3B6PLJ3) | M8CG24) |
| Brachypodium | 26.033 | | | | |
| distachyon: | | | | | |
| (UniProt: | | | | | |
| I1IB35) | | | | | |
| Hordeum | 29.098 | 55.645 | | | |
| vulgare | | | | | |
| subsp. | | | | | |
| vulgare: | | | | | |
| (UniProt: | | | | | |
| F2EGV1) | | | | | |
| Triticum | 29.614 | 60.169 | 86.192 | | |
| aestivum: | | | | | |
| (UniProt: | | | | | |
| A0A3B6PLJ3) | | | | | |
| Aegilops | 28.755 | 59.322 | 82.427 | 93.805 | |
| tauschii: | | | | | |
| (UniProt: | | | | | |
| M8CG24) | | | | | |
| Sorghum | 25.726 | 47.458 | 45.565 | 48.729 | 49.153 |
| bicolor: | | | | | |
| (UniProt: | | | | | |
| C5XWE8) | | | | | |
| Oryza | 29.596 | 47.28 | 44.262 | 46.154 | 46.154 |
| sativa: | | | | | |
| (RefSeq: | | | | | |
| XP_015635088) | | | | | |
| Zea mays: | 27.778 | 41.975 | 41.736 | 42.553 | 42.979 |
| (UniProt: | | | | | |
| B4FG75) | | | | | |
| Zea mays: | 26.407 | 51.037 | 48.594 | 51.055 | 52.321 |
| (GenBank: AQK7 | | | | | |
| 2186.1) | | | | | |
| Zea mays: | 22.4 | 44.715 | 40.385 | 42.972 | 43.373 |
| (GenBank: | | | | | |
| AQK72188.1) | | | | | |

| | Sorghum | Oryza | Zea | |
|---|---|---|---|---|
| OSD1 | bicolor: | sativa: | mays: | Zea mays: |
| Monocot % | (UniProt: | (RefSeq: | (UniProt: | (GenBank: |
| ID | C5XWE8) | XP015635088) | B4FG75) | AQK72186.1) |
| Brachypodium | | | | |
| distachyon: | | | | |
| (UniProt: | | | | |
| I1IB35) | | | | |
| Hordeum | | | | |
| vulgare | | | | |
| subsp. | | | | |
| vulgare: | | | | |
| (UniProt: | | | | |
| F2EGV1) | | | | |
| Triticum | | | | |
| aestivum: | | | | |
| (UniProt: | | | | |
| A0A3B6PLJ3) | | | | |
| Aegilops | | | | |
| tauschii: | | | | |
| (UniProt: | | | | |
| M8CG24) | | | | |
| Sorghum | | | | |
| bicolor: | | | | |
| (UniProt: | | | | |
| C5XWE8) | | | | |
| Oryza | 48.101 | | | |
| sativa: | | | | |
| (RefSeq: | | | | |
| XP_015635088) | | | | |
| Zea mays: | 40.664 | 55.508 | | |
| (UniProt: | | | | |
| B4FG75) | | | | |
| Zea mays: | 54.393 | 54.545 | 47.083 | |
| (GenBank: AQK7 | | | | |
| 2186.1) | | | | |
| Zea mays: | 72.764 | 42.276 | 38.34 | 50 |
| (GenBank: | | | | |
| AQK72188.1) | | | | |

SEQUENCE ALIGNMENT 9
CYCA1 protein sequences from monocotyledonous plants.
CYCA1 Monocots
Homologs identified by BLAST of Arabidopsis thaliana [symbol]
map to monocot NCBI RefSeq, GenBank and UniProtKB protein datatbases.

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Zea_mays:(RefSeq:NP_ [symbol] ) | [symbol] |
| Zea_mays:(RefSeq:NP_ [symbol] ) | [symbol] |
| Dryza_sativa_japonica_Group:(RefSeq:XP_ [symbol] ) | [symbol] |
| Sorghum_bicolor:(RefSeq:XP_ [symbol] ) | [symbol] |
| Brachypodium_distachyon:(RefSeq:XP_ [symbol] ) | [symbol] |
| Brachypodium_distachyon:(RefSeq:XP_ [symbol] ) | [symbol] |
| Aegilops_tauschii_subsp._strangulata:(RefSeq: [symbol] ) | [symbol] |
| Oryza_sativa_Japonica_Group:(RefSeq:XP_ [symbol] ) | [symbol] |
| Hordeum_vulgare_subsp._vulgare:(XP_ [symbol] ) | [symbol] |
| Triticum_aestivum:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_ [symbol] ) | [symbol] |
| Sorghum_bicolor:(RefSeq:XP_ [symbol] ) | [symbol] |
| Musa_acuminata_subsp._malaccensis:(RefSeq:XP_ [symbol] ) | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Zea_mays:(RefSeq:NP_ [symbol] ) | [symbol] |
| Zea_mays:(RefSeq:NP_ [symbol] ) | [symbol] |
| Dryza_sativa_japonica_Group:(RefSeq:XP_ [symbol] ) | [symbol] |
| Sorghum_bicolor:(RefSeq:XP_ [symbol] ) | [symbol] |
| Brachypodium_distachyon:(RefSeq:XP_ [symbol] ) | [symbol] |
| Brachypodium_distachyon:(RefSeq:XP_ [symbol] ) | [symbol] |
| Aegilops_tauschii_subsp._strangulata:(RefSeq: [symbol] ) | [symbol] |
| Oryza_sativa_Japonica_Group:(RefSeq:XP_ [symbol] ) | [symbol] |
| Hordeum_vulgare_subsp._vulgare:(XP_ [symbol] ) | [symbol] |
| Triticum_aestivum:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_ [symbol] ) | [symbol] |
| Sorghum_bicolor:(RefSeq:XP_ [symbol] ) | [symbol] |
| Musa_acuminata_subsp._malaccensis:(RefSeq:XP_ [symbol] ) | [symbol] |
| | 210 220 230 240 250 260 270 280 290 300 |
| | ----:----\|----:----\|----:----\|----:----\|----:----\|----:----\|----:----\|----:----\|----:----\|----:----\| |
| Consensus | [symbol] |
| Zea_mays:(RefSeq:NP_ [symbol] ) | [symbol] |
| Zea_mays:(RefSeq:NP_ [symbol] ) | [symbol] |
| Dryza_sativa_japonica_Group:(RefSeq:XP_ [symbol] ) | [symbol] |
| Sorghum_bicolor:(RefSeq:XP_ [symbol] ) | [symbol] |
| Brachypodium_distachyon:(RefSeq:XP_ [symbol] ) | [symbol] |
| Brachypodium_distachyon:(RefSeq:XP_ [symbol] ) | [symbol] |
| Aegilops_tauschii_subsp._strangulata:(RefSeq: [symbol] ) | [symbol] |
| Oryza_sativa_Japonica_Group:(RefSeq:XP_ [symbol] ) | [symbol] |
| Hordeum_vulgare_subsp._vulgare:(XP_ [symbol] ) | [symbol] |
| Triticum_aestivum:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_ [symbol] ) | [symbol] |
| Sorghum_bicolor:(RefSeq:XP_ [symbol] ) | [symbol] |
| Musa_acuminata_subsp._malaccensis:(RefSeq:XP_ [symbol] ) | [symbol] |
| | 310 320 330 340 350 360 370 380 390 400 |
| | ----:----\|----:----\|----:----\|----:----\|----:----\|----:----\|----:----\|----:----\|----:----\|----:----\| |
| Consensus | [symbol] |
| Zea_mays:(RefSeq:NP_ [symbol] ) | [symbol] |
| Zea_mays:(RefSeq:NP_ [symbol] ) | [symbol] |
| Dryza_sativa_japonica_Group:(RefSeq:XP_ [symbol] ) | [symbol] |
| Sorghum_bicolor:(RefSeq:XP_ [symbol] ) | [symbol] |
| Brachypodium_distachyon:(RefSeq:XP_ [symbol] ) | [symbol] |
| Brachypodium_distachyon:(RefSeq:XP_ [symbol] ) | [symbol] |
| Aegilops_tauschii_subsp._strangulata:(RefSeq: [symbol] ) | [symbol] |
| Oryza_sativa_Japonica_Group:(RefSeq:XP_ [symbol] ) | [symbol] |
| Hordeum_vulgare_subsp._vulgare:(XP_ [symbol] ) | [symbol] |
| Triticum_aestivum:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_ [symbol] ) | [symbol] |
| Sorghum_bicolor:(RefSeq:XP_ [symbol] ) | [symbol] |
| Musa_acuminata_subsp._malaccensis:(RefSeq:XP_ [symbol] ) | [symbol] |
| | 410 420 430 440 450 460 470 480 490 500 |
| | ----:----\|----:----\|----:----\|----:----\|----:----\|----:----\|----:----\|----:----\|----:----\|----:----\| |
| Consensus | [symbol] |
| Zea_mays:(RefSeq:NP_ [symbol] ) | [symbol] |
| Zea_mays:(RefSeq:NP_ [symbol] ) | [symbol] |
| Dryza_sativa_japonica_Group:(RefSeq:XP_ [symbol] ) | [symbol] |
| Sorghum_bicolor:(RefSeq:XP_ [symbol] ) | [symbol] |
| Brachypodium_distachyon:(RefSeq:XP_ [symbol] ) | [symbol] |
| Brachypodium_distachyon:(RefSeq:XP_ [symbol] ) | [symbol] |
| Aegilops_tauschii_subsp._strangulata:(RefSeq: [symbol] ) | [symbol] |
| Oryza_sativa_Japonica_Group:(RefSeq:XP_ [symbol] ) | [symbol] |
| Hordeum_vulgare_subsp._vulgare:(XP_ [symbol] ) | [symbol] |
| Triticum_aestivum:(RefSeq:XP_ [symbol] ) | [symbol] |
| Oryza_sativa_Japonica_Group:(RefSeq:XP_ [symbol] ) | [symbol] |
| Sorghum_bicolor:(RefSeq:XP_ [symbol] ) | [symbol] |
| Sorghum_bicolor:(RefSeq:XP_ [symbol] ) | [symbol] |
| Musa_acuminata_subsp._malaccensis:(RefSeq:XP_ [symbol] ) | [symbol] |
| | 510 520 530 |
| | ----:----\|----:----\|----:----\|- |
| Consensus | [symbol] |
| Zea_mays:(RefSeq:NP_ [symbol] ) | ...................Q..T.....T.. |
| Zea_mays:(RefSeq:NP_ [symbol] ) | ...................Q.........A. |
| Dryza_sativa_japonica_Group:(RefSeq:XP_ [symbol] ) | ....T..........P......T........ |
| Sorghum_bicolor:(RefSeq:XP_ [symbol] ) | ...................Q..T........ |
| Brachypodium_distachyon:(RefSeq:XP_ [symbol] ) | ...................LV..D....... |
| Brachypodium_distachyon:(RefSeq:XP_ [symbol] ) | ...........A.......MV..DY.C..A. |
| Aegilops_tauschii_subsp._strangulata:(RefSeq: [symbol] ) | ............G.....T.V........A. |
| Oryza_sativa_Japonica_Group:(RefSeq:XP_ [symbol] ) | ....T..........H....V.S........ |
| Hordeum_vulgare_subsp._vulgare:(XP_ [symbol] ) | ............G.....T.V........A. |
| Triticum_aestivum:(RefSeq:XP_ [symbol] ) | ............G.....T.V........A. |
| Oryza_sativa_Japonica_Group:(RefSeq:XP_ [symbol] ) | ....T..........P......T.....S.. |
| Sorghum_bicolor:(RefSeq:XP_ [symbol] ) | ...................Q..T.....T.- |
| Sorghum_bicolor:(RefSeq:XP_ [symbol] ) | .............R........T.....V.M |
| Musa_acuminata_subsp._malaccensis:(RefSeq:XP_ [symbol] ) | ...............Y..A.....Y.Q..RN |

[symbol]

indicates data missing or illegible when filed

TABLE 8A Percent identities of CYCA1 sequences from monocotyledonous plants.

| | | | | Sorghum_{—} | |
|---|---|---|---|---|---|
| | Zea_{—} | Zea_{—} | Oryza_sativa_{—} | bicolor: | Brachypodium_{—} |
| | mays: | mays: | Japonica_Group: | (RefSeq: | distachyon: |
| TAM (CYCA1) | (RefSeq: | (RefSeq: | (RefSeq: | XP_{—} | (RefSeq: |
| monocot % ID | NP_001288521.1) | NP_001105387.2) | XP_015630726.1) | 002455447.1) | XP003566077.1) |
| Zea_mays: | 80.271 | | | | |
| (RefSeq: NP_001105387.2) | | | | | |
| Oryza_sativa_Japonica_Group: | 74.618 | 75.483 | | | |
| (RefSeq: XP_015630726.1) | | | | | |
| Sorghum_bicolor: | 90.196 | 80.897 | 76.923 | | |
| (RefSeq: XP_002455447.1) | | | | | |
| Brachypodium_distachyon: | 72.692 | 72.763 | 77.457 | 73.359 | |
| (RefSeq: XP_003566077.1) | | | | | |
| Brachypodium_distachyon: | 66.667 | 67.446 | 71.869 | 67.636 | 81.518 |
| (RefSeq: XP_003566081.1) | | | | | |
| Aegilops_tauschii_{—} | 72.692 | 75.292 | 79.191 | 74.517 | 81.518 |
| subsp._strangulata: | | | | | |
| (RefSeq: XP_020187359.1) | | | | | |
| Oryza_sativa_Japonica | 72.394 | 73.529 | 88.78 | 75.049 | 76.07 |
| Group: (RefSeq: | | | | | |
| XP_025879970.1) | | | | | |
| Hordeum_vulgare_{—} | 72.137 | 74.324 | 79.271 | 73.563 | 82.592 |
| subsp._vulgare: | | | | | |
| (XP_044975533.1) | | | | | |
| Triticum_aestivum: | 72.692 | 74.757 | 78.846 | 74.71 | 81.942 |
| (Ref Seq: | | | | | |
| XP_044356181.1) | | | | | |
| Oryza_sativa_Japonica | 74.419 | 75.342 | 89.784 | 76.953 | 76.311 |
| Group: (RefSeq: | | | | | |
| XP_025878069.1) | | | | | |
| Sorghum_bicolor: | 82.364 | 86.22 | 77.264 | 84.932 | 74.806 |
| (RefSeq: | | | | | |
| XP_002454880.1) | | | | | |
| Sorghum_bicolor: | 77.649 | 79.183 | 73.18 | 78.876 | 72.78 |
| (RefSeq: | | | | | |
| XP_021311446.1) | | | | | |
| Musa_acuminata_subsp._{—} | 61.315 | 62.109 | 63.938 | 62.451 | 61.644 |
| malaccensis: (RefSeq: | | | | | |
| XP_018678008.1) | | | | | |

| | | Aegilops_{—} | | |
|---|---|---|---|---|
| | | tauschii_{—} | | Hordeum_{—} |
| | Brachypodium_{—} | subsp._{—} | Oryza_sativa | vulgare_{—} |
| | distachyon: | strangulata: | Japonica_Group: | subsp._{—} |
| TAM (CYCA1) | (RefSeq: | (RefSeq: | (RefSeq: | vulgare: |
| monocot % ID | XP003566081.1) | XP_020187359.1) | XP_025879970.1) | (XP_044975533.1) |
| Zea_mays: | | | | |
| (RefSeq: NP_001105387.2) | | | | |
| Oryza_sativa_Japonica_Group: | | | | |
| (RefSeq: XP_015630726.1) | | | | |
| Sorghum_bicolor: | | | | |
| (RefSeq: XP_002455447.1) | | | | |
| Brachypodium_distachyon: | | | | |
| (RefSeq: XP_003566077.1) | | | | |
| Brachypodium_distachyon: | | | | |
| (RefSeq: XP_003566081.1) | | | | |
| Aegilops_tauschii_{—} | 76.998 | | | |
| subsp._strangulata: | | | | |
| (RefSeq: XP_020187359.1) | | | | |
| Oryza_sativa_Japonica | 70.76 | 77.626 | | |
| Group: (RefSeq: | | | | |
| XP_025879970.1) | | | | |
| Hordeum_vulgare_{—} | 78.101 | 93.992 | 77.799 | |
| subsp._vulgare: | | | | |
| (XP_044975533.1) | | | | |
| Triticum_aestivum: | 77.043 | 98.826 | 77.67 | 94.186 |
| (Ref Seq: | | | | |
| XP_044356181.1) | | | | |
| Oryza_sativa_Japonica | 71.401 | 79.029 | 91.498 | 77.992 |
| Group: (RefSeq: | | | | |
| XP_025878069.1) | | | | |
| Sorghum_bicolor: | 68.544 | 76.311 | 75.734 | 74.952 |
| (RefSeq: | | | | |
| XP_002454880.1) | | | | |
| Sorghum_bicolor: | 66.538 | 71.622 | 72.32 | 71.401 |
| (RefSeq: | | | | |
| XP_021311446.1) | | | | |
| Musa_acuminata_subsp._{—} | 59.687 | 62.695 | 63.636 | 62.33 |
| malaccensis: (RefSeq: | | | | |
| XP_018678008.1) | | | | |

| | Triticum_{—} | Oryza_sativa_{—} | Sorghum_{—} | Sorghum_{—} |
|---|---|---|---|---|
| | aestivum: | Japonica_Group: | bicolor: | bicolor: |
| TAM (CYCA1) | (RefSeq: | (RefSeq: | (RefSeq: | (RefSeq: |
| monocot % ID | XP_044356181.1) | XP_025878069.1) | XP_002454880.1) | XP_021311446.1) |
| Zea_mays: | | | | |
| (RefSeq: NP_001105387.2) | | | | |
| Oryza_sativa_{—} | | | | |
| Japonica_Group: | | | | |
| (RefSeq: | | | | |
| XP_015630726.1) | | | | |
| Sorghum_bicolor: | | | | |
| (RefSeq: XP_002455447.1) | | | | |
| Brachypodium_distachyon: | | | | |
| (RefSeq: XP_003566077.1) | | | | |
| Brachypodium_distachyon: | | | | |
| (RefSeq: XP_003566081.1) | | | | |
| Aegilops_tauschii_{—} | | | | |
| subsp._strangulata: | | | | |
| (RefSeq: XP_020187359.1) | | | | |
| Oryza_sativa_Japonica | | | | |
| Group: (RefSeq: | | | | |
| XP_025879970.1) | | | | |
| Hordeum_vulgare_{—} | | | | |
| subsp._vulgare: | | | | |
| (XP_044975533.1) | | | | |
| Triticum_aestivum: | | | | |
| (Ref Seq: | | | | |
| XP_044356181.1) | | | | |
| Oryza_sativa_Japonica | 79.377 | | | |
| Group: (RefSeq: | | | | |
| XP_025878069.1) | | | | |
| Sorghum_bicolor: | 76.848 | 78.193 | | |
| (RefSeq: | | | | |
| XP_002454880.1) | | | | |
| Sorghum_bicolor: | 72.147 | 73.633 | 82.813 | |
| (RefSeq: | | | | |
| XP_021311446.1) | | | | |
| Musa_acuminata_subsp._{—} | 63.014 | 64.484 | 63.796 | 61.719 |
| malaccensis: (RefSeq: | | | | |
| XP_018678008.1) | | | | |

SEQUENCE ALIGNMENT 10
CYCA1 protein sequences from dicotyledonous plants.
CYCA1 Dicots
Homologs identified by Blast of Arabidopsis thaliana [symbol] [symbol]
(not included in this alignment) [symbol] v1.0 protein databases

| | |
|---|---|
| | 10 20 30 40 50 60 70 80 90 100 |
| | ----:----\|----:----\|----:----\|----:----\|----:----\|----:----\|----:----\|----:----\|----:----\|----:----\| |
| Consensus | [symbol] |
| Citrullus lanatus: [symbol] | [symbol] |
| Vaccinium corymbosum: [symbol] | [symbol] |
| Solanum lycopersicum: [symbol] | [symbol] |
| Arabidopsis thaliana: [symbol] | [symbol] |
| Gossypium barbadense: [symbol] | [symbol] |
| Prunus avium: [symbol] | [symbol] |
| Solnum tuberosum: [symbol] | [symbol] |
| | 110 120 130 140 150 160 170 180 190 200 |
| | ----:----\|----:----\|----:----\|----:----\|----:----\|----:----\|----:----\|----:----\|----:----\|----:----\| |
| Consensus | [symbol] |
| Citrullus lanatus: [symbol] | [symbol] |
| Vaccinium corymbosum: [symbol] | [symbol] |
| Solanum lycopersicum: [symbol] | [symbol] |
| Arabidopsis thaliana: [symbol] | [symbol] |
| Gossypium barbadense: [symbol] | [symbol] |
| Prunus avium: [symbol] | [symbol] |
| Solnum tuberosum: [symbol] | [symbol] |
| | 210 220 230 240 250 260 270 280 290 300 |
| | ----:----\|----:----\|----:----\|----:----\|----:----\|----:----\|----:----\|----:----\|----:----\|----:----\| |
| Consensus | [symbol] |
| Citrullus lanatus: [symbol] | [symbol] |
| Vaccinium corymbosum: [symbol] | [symbol] |
| Solanum lycopersicum: [symbol] | [symbol] |
| Arabidopsis thaliana: [symbol] | [symbol] |
| Gossypium barbadense: [symbol] | [symbol] |
| Prunus avium: [symbol] | [symbol] |
| Solnum tuberosum: [symbol] | [symbol] |
| | 310 320 330 340 350 360 370 380 390 400 |
| | ----:----\|----:----\|----:----\|----:----\|----:----\|----:----\|----:----\|----:----\|----:----\|----:----\| |
| Consensus | [symbol] |
| Citrullus lanatus: [symbol] | [symbol] |
| Vaccinium corymbosum: [symbol] | [symbol] |
| Solanum lycopersicum: [symbol] | [symbol] |
| Arabidopsis thaliana: [symbol] | [symbol] |
| Gossypium barbadense: [symbol] | [symbol] |
| Prunus avium: [symbol] | [symbol] |
| Solnum tuberosum: [symbol] | [symbol] |
| | 410 420 430 440 450 460 470 480 490 500 |
| | ----:----\|----:----\|----:----\|----:----\|----:----\|----:----\|----:----\|----:----\|----:----\|----:----\| |
| Consensus | [symbol] |
| Citrullus lanatus: [symbol] | [symbol] |
| Vaccinium corymbosum: [symbol] | [symbol] |
| Solanum lycopersicum: [symbol] | [symbol] |
| Arabidopsis thaliana: [symbol] | [symbol] |
| Gossypium barbadense: [symbol] | [symbol] |
| Prunus avium: [symbol] | [symbol] |
| Solnum tuberosum: [symbol] | [symbol] |
| | 510 |
| | ----:----\|----: |
| Consensus | [symbol] |
| Citrullus lanatus: [symbol] | [symbol] |
| Vaccinium corymbosum: [symbol] | [symbol] |
| Solanum lycopersicum: [symbol] | [symbol] |
| Arabidopsis thaliana: [symbol] | [symbol] |
| Gossypium barbadense: [symbol] | [symbol] |
| Prunus avium: [symbol] | [symbol] |
| Solnum tuberosum: [symbol] | [symbol] |

[symbol]

indicates data missing or illegible when filed

TABLE 8B
Percent identities of CYCAI sequences trom dicotyledonous plants.

| | | Vaccinium | | | | |
|---|---|---|---|---|---|---|
| | | corymbosum: | Solanum | Arabidopsis | | |
| | | (VaccDscaff | lycopersicum: | thaliana: | Gossypium | Prunus |
| | Citrullus | 2-augustus- | (RefSeq: | (Ref Seq: | barbadense: | avium: |
| TAM (CYCA1) | lanatus: | gene-199.26- | NP_001233762. | NP_177 | (UniProt: | (RefSeq: |
| Dicot % ID | (Cla97C08G148690.1) | mRNA-1) | ) | 863.2) | A0A2P5VPW8) | XP_021806552.1) |
| Vaccinium | 63.511 | | | | | |
| corymbosum: | | | | | | |
| (VaccDscaff2- | | | | | | |
| augustus-gene- | | | | | | |
| 199.26-mRNA-1) | | | | | | |
| Solanum | 59.646 | 66.2 | | | | |
| lycopersicum: (Ref | | | | | | |
| Seq: NP_001233762.1) | | | | | | |
| Arabidopsis | 44.99 | 45.984 | 44.758 | | | |
| thaliana: (RefSeq: | | | | | | |
| NP_177863.2) | | | | | | |
| Gossypium | 67.647 | 64.729 | 63.4 | 47.581 | | |
| barbadense: | | | | | | |
| (UniProt: | | | | | | |
| A0A2P5VPW8) | | | | | | |
| Prunus | 68.102 | 64.95 | 64.44 | 47.515 | 67.061 | |
| avium: (RefSeq: | | | | | | |
| XP_021806552.1) | | | | | | |
| Solanum | 59.961 | 65.531 | 93.347 | 44.89 | 63.783 | 64.032 |
| tuberosum: | | | | | | |
| (RefSeq: | | | | | | |
| XP_006351136.1) | | | | | | |

TABLE 9
Table of SEQ ID NOs.

| SEQ | | | |
|---|---|---|---|
| ID | | Plant | |
| NO. | Sequence | Type | Sequence Source (Accession) |
| 1 | REC8 protein | dicot | Gossypium barbadense |
| | | | (UniProt: A0A5J5VWS8) |
| 2 | REC8 protein | dicot | Arabidopsis thaliana |
| | | | (UniProt: Q9S7T7) |
| 3 | REC8 protein | dicot | Rubus occidentalis |
| | | | (MAKER¹: Ro05_G04582) |
| 4 | REC8 protein | dicot | Citrullus lanatus |
| | | | (MAKER¹: C1CG07G004460.1) |
| 5 | REC8 protein | dicot | Prunus avium |
| | | | (UniProt: A0A6P5RV91) |
| 6 | REC8 protein | dicot | Vaccinium corymbosum |
| | | | (MAKER¹: VaccDscaff20- |
| | | | snap-gene-38.40-mRNA-1) |
| 7 | REC8 protein | dicot | Solanum lycopersicum |
| | | | (RefSeq: XP_025887480.1) |
| 8 | REC8 protein | dicot | Solanum tuberosum |
| | | | (RefSeq: XP_006347252.1) |
| 9 | REC8 protein | dicot | REC8.dicot Alignment |
| | | | consensus sequence |
| | | | (Sequence Alignment 1) |
| 10 | REC8 protein | monocot | Musa acuminata subsp. malaccensis |
| | | | (RefSeq: XP_018681669.1) |
| 11 | REC8 protein | monocot | Zea mays |
| | | | (RefSeq: XP_008648328.1) |
| 12 | REC8 protein | monocot | Sorghum bicolor |
| | | | (RefSeq: XP_021302730.1) |
| 13 | REC8 protein | monocot | Oryza sativa |
| | | | (RefSeq: XP_015638092.1) |
| 14 | REC8 protein | monocot | Brachypodium distachyon |
| | | | (RefSeq: XP_003567819.1) |
| 15 | REC8 protein | monocot | Hordeum vulgare subsp. vulgare |
| | | | (UniProt: A0A287GQN1)* |
| | | | XP_044984305.1 |
| 16 | REC8 protein | monocot | Triticum aestivum |
| | | | (UniProt: A0A3B5Z6B7) |
| 17 | REC8 protein | monocot | Aegilops tauschii subsp. tauschii |
| | | | (RefSeq: XP_020178692.1) |
| 18 | REC8 protein | monocot | REC8.monocot |
| | | | Alignment consensus |
| | | | sequence (Sequence Alignment 2) |
| 19 | SPO11-1 | dicot | Citrullus lanatus |
| | protein | | (Cla97C04G075440.1)* |
| | | | ClCG09G021210.1 |
| 20 | SPO11-1 | dicot | Rubus occidentalis |
| | protein | | (Ro01 G11692)*Ro03_G32130 |
| 21 | SPO11-1 | dicot | Vaccinium corymbosum |
| | protein | | (MAKER¹: VaccDscaff83- |
| | | | snap-gene-5.35-mRNA-1) |
| 22 | SPO11-1 | dicot | Arabidopsis thaliana |
| | protein | | (UniProt: Q9M4A2) |
| 23 | SPO11-1 | dicot | Gossypium barbadense |
| | protein | | (UniProt: A0A5J5SLP4) |
| 24 | SPO11-1 | dicot | Prunus avium |
| | protein | | (UniProt: A0A6P5TTD7) |
| 25 | SPO11-1 | dicot | Solanum tuberosum |
| | protein | | (RefSeq: XP_006346146.1) |
| 26 | SPO11-1 | dicot | Solanum lycopersicum |
| | protein | | (RefSeq: XP_010324270.1) |
| 27 | SPO11-1 | dicot | SPO11-1.dicot |
| | | | Alignment consensus |
| | protein | | sequence (Sequence Alignment 3) |
| 28 | SPO11-1 | monocot | Musa_acuminata_ |
| | protein | | subsp._malaccensis |
| | | | (XP_009380133.2) |
| 29 | SPO11-1 | monocot | Zea mays |
| | protein | | (RefSeq: NP_001347894.11) |
| 30 | SPO11-1 | monocot | Sorghum bicolor |
| | protein | | (RefSeq: XP_002466440.1) |
| 31 | SPO11-1 | monocot | Oryza sativa Japonica Group |
| | protein | | (RefSeq: NP_001391702.1) |
| 32 | SPO11-1 | monocot | Aegilops tauschii |
| | protein | | (RefSeq: XP_020158624.1) |
| 33 | SPO11-1 | monocot | Brachypodium distachyon |
| | protein | | (RefSeq: XP_003559449.1) |
| 34 | SPO11-1 | monocot | Triticum aestivum |
| | protein | | (UniProt: A0A3B6LTD1) |
| 35 | SPO11-1 | monocot | Hordeum vulgare subsp. vulgare |
| | protein | | (UniProt: A0A287SD60) |
| 36 | SPO11-1 | monocot | SPO11-1.monocot |
| | | | Alignment consensus |
| | protein | | sequence (Sequence Alignment 4) |
| 37 | PAIR1 protein | dicot | Arabidopsis thaliana |
| | | | (RefSeq: NP_001318904.1) |
| 38 | PAIR1 protein | dicot | Citrullus lanatus |
| | | | (MAKER¹: Cla97C05G081880.1) |
| 39 | PAIR1 protein | dicot | Solanum tuberosum |
| | | | (RefSeq: XP_006339791.2) |
| 40 | PAIR1 protein | dicot | Solanum lycopersicum |
| | | | (RefSeq: XP_004231186.2) |
| 41 | PAIR1 protein | dicot | Vaccinium corymbosum |
| | | | (MAKER¹: VaccDscaff40- |
| | | | augustus-gene-183.9-mRNA-1) |
| 42 | PAIR1 protein | dicot | Gossypium barbadense |
| | | | (UniProt: A0A5J5QG99) |
| 43 | PAIR1 protein | dicot | Prunus avium |
| | | | (UniProt: A0A6P5RCR3) |
| 44 | PAIR1 protein | dicot | Rubus occidentalis |
| | | | (MAKER¹: Ro07_G04309) |
| 45 | PAIR1 protein | dicot | PAIR1.dicot Alignment |
| | | | consensus sequence |
| | | | (Sequence Alignment 5) |
| 46 | PAIR1 protein | monocot | Musa acuminata subsp. malaccensis |
| | | | (RefSeq: XP_018685297.1) |
| 47 | PAIR1 protein | monocot | Triticum aestivum |
| | | | (UniProt: A0A3B6C7X9) |
| 48 | PAIR1 protein | monocot | Hordeum vulgare subsp. vulgare |
| | | | (UniProt: A0A287Q0E5)* |
| | | | (RefSeq: XP_044983266.1) |
| 49 | PAIR1 protein | monocot | Aegilops tauschii |
| | | | (UniProt: N1R2L9) |
| 50 | PAIR1 protein | monocot | Brachypodium distachyon |
| | | | (RefSeq: XP_014751501.1) |
| 51 | PAIR1 protein | monocot | Sorghum bicolor |
| | | | (UniProt: A0A1B6QR58) |
| 52 | PAIR1-1 | monocot | Zea mays |
| | protein | | (RefSeq: XP_008660580.1) |
| 53 | PAIR1-2 | monocot | Zea mays |
| | protein | | (RefSeq: ONL92656.1) |
| 54 | PAIR1 protein | monocot | Oryza sativa Japonica Group |
| | | | (RefSeq: XP_015632198.1) |
| 55 | PAIR1 protein | monocot | PAIR1.monocot |
| | | | Alignment consensus |
| | | | sequence (Sequence Alignment 6) |
| 56 | OSD1 protein | dicot | Prunus avium |
| | | | (RefSeq: XP_021833341.1) |
| 57 | OSD1 protein | dicot | Rubus occidentalis |
| | | | (MAKER¹: Ro06_G14226) |
| 58 | OSD1 protein | dicot | Arabidopsis thaliana |
| | | | (UniProt: Q9M2R1) |
| 59 | OSD1 protein | dicot | Gossypium barbadense |
| | | | (UniProt: A0A2P5XW92) |
| 60 | OSD1 protein | dicot | Citrullus lanatus |
| | | | (MAKER¹: Cla97C04G068720.1) |
| 61 | OSD1 protein | dicot | Vaccinium corymbosum |
| | | | (MAKER¹: VaccDscaff5- |
| | | | snap-gene-277.41- |
| | | | mRNA-1) |
| 62 | OSD1 protein | dicot | Solanum lycopersicum |
| | | | (UniProt: A0A3Q7ILG3) |
| 63 | OSD1 protein | dicot | Solanum tuberosum |
| | | | (RefSeq: XP_006351336.1) |
| 64 | OSD1 protein | dicot | OSD1.dicot Alignment |
| | | | consensus sequence |
| | | | (Sequence Alignment 7) |
| 65 | OSD1 protein | monocot | Musa acuminata |
| | | | (RefSeq: XP_009415567) |
| 66 | OSD1 protein | monocot | Brachypodium distachyon |
| | | | (UniProt: I1IB35) |
| 67 | OSD1 protein | monocot | Hordeum vulgare subsp. vulgare |
| | | | (UniProt: F2EGV1) |
| 68 | OSD1 protein | monocot | Triticum aestivum |
| | | | (UniProt: A0A3B6PLJ3) |
| 69 | OSD1 protein | monocot | Aegilops tauschii |
| | | | (UniProt: M8CG24) |
| 70 | OSD1 protein | monocot | Sorghum bicolor |
| | | | (UniProt: C5XWE8) |
| 71 | OSD1 protein | monocot | Oryza sativa |
| | | | (RefSeq: XP_015635088) |
| 72 | OSD1-1 protein | monocot | Zea mays OSD1 on chromosome 2 |
| | | | (“ZmOsd1-chr2”) |
| | | | (UniProt: B4FG75) |
| 73 | OSD1-2 protein | monocot | Zea mays OSD1 on chromosome 5 |
| | | | (“ZmOsd1-chr5-1”) |
| | | | (GenBank: AQK72186.1) |
| 74 | OSD1-3 protein | monocot | Zea mays OSD1 |
| | | | duplication on chromosome |
| | | | 5 (“ZmOsd1-chr5-2”) |
| | | | (GenBank: AQK72188.1) |
| 75 | OSD1 protein | monocot | OSD1.monocot |
| | | | Alignment consensus |
| | | | sequence (Sequence Alignment 8) |
| 76 | JASON-1 | monocot | Zea mays |
| | protein | | (RefSeq: NP_001130670.1) |
| 77 | JASON-2 | monocot | Zea mays |
| | protein | | (RefSeq: NP_001130438.1) |
| 78 | CYCLIN-A1 | monocot | Zea mays |
| | (CYCA1) | | (GenBank: NP_001288521.1) |
| | protein | | |
| 79 | TDM1 protein | monocot | Zea mays |
| | | | (RefSeq: NP_001141923.1) |
| 80 | PS1 protein | monocot | Zea mays |
| | | | (RefSeq: NP_001348386.1) |
| 81 | CYCA1-1 | dicot | Solanum tuberosum |
| | protein | | (RefSeq: XP_006351136.1) |
| 82 | CYCA1-2 | dicot | Solanum tuberosum |
| | protein | | (RefSeq: XP_006351137.1) |
| 83 | CYCA1-3 | dicot | Solanum tuberosum |
| | protein | | (RefSeq: XP_006351138.1) |
| 84 | TDM1 protein | dicot | Solanum tuberosum |
| | | | (RefSeq: XP_006350746.1) |
| 85 | PS1 protein | dicot | Solanum tuberosum |
| | | | (RefSeq: XP_006351164.1) |
| 86 | PS1-LIKE | dicot | Solanum tuberosum |
| | PROTEIN | | (RefSeq: XP_006353632.1) |
| 87 | JASON protein | dicot | Solanum tuberosum |
| | | | (RefSeq: XP_006352001.1) |
| 88 | REC8 protein | dicot | Glycine max |
| | | | (RefSeq: XP_006575554.1) |
| 89 | SPO11-1 | dicot | Glycine max |
| | protein | | (RefSeq: XP_040865641.1) |
| 90 | PAIR1 protein | dicot | Glycine max |
| | | | (RefSeq: XP_006583295.1) |
| 91 | OSD1-1 protein | dicot | Glycine max |
| | | | (RefSeq: XP_003521198.1) |
| 92 | OSD1-2 protein | dicot | Glycine max |
| | | | (RefSeq: XP_006604381.1) |
| 93 | TAM/CYCLIN- | dicot | Glycine max |
| | A1(CYCA1) | | (RefSeq: XP_003526649.1) |
| | protein | | |
| 94 | TDM1-1 | dicot | Glycine max |
| | protein | | (RefSeq: XP_006586848.1) |
| 95 | TDM1-2 | dicot | Glycine max |
| | protein | | (RefSeq: XP_003547331.2) |
| 96 | CYCLIN-A1 | dicot | Arabidopsis thaliana |
| | (CYCA1) | | (RefSeq: NP_177863.2) |
| | protein | | |
| 97 | CYCLIN-A1 | dicot | Solanum tuberosum |
| | (CYCA1) | | (RefSeq: XP_006351136.1) |
| | protein | | |
| 98 | CYCLIN-A1 | dicot | Citrullus lanatus |
| | (CYCA1) | | (MAKER¹: Cla97C080148690.1) |
| | protein | | |
| 99 | CYCLIN-A1 | dicot | Prunus avium |
| | (CYCA1) | | (RefSeq: XP_021806552.1) |
| | protein | | |
| 100 | CYCLIN-A1 | dicot | Vaccinium corymbosum |
| | (CYCA1) | | (MAKER¹: VaccDscaff2- |
| | protein | | augustus-gene-199.26-mRNA-1) |
| 101 | CYCLIN-A1 | dicot | Solanum lycopersicum |
| | (CYCA1) | | (RefSeq: NP_001233762.1) |
| | protein | | |
| 102 | CYCLIN-A1 | dicot | Gossypium barbadense |
| | (CYCA1) | | (UniProt: A0A2P5VPW8) |
| | protein | | |
| 103 | CYCLIN-A1 | dicot | Rubus occidentalis |
| | (CYCA1) | | (MAKER¹: Ro02_G01483) |
| | protein | | |
| 104 | CYCLIN-A1 | dicot | CYCA1 dicot Alignment |
| | (CYCA1) | | consensus sequence |
| | protein | | (Sequence Alignment 18) |
| 105 | CYCLIN-A1-1 | monocot | Zea mays CYCA1 |
| | (CYCA1-1) | | on chromosome 3 |
| | | | (RefSeq: NP_001288521.1) |
| 106 | CYCLIN-A1-2 | monocot | Zea mays CYCA1 |
| | (CYCA1-2) | | on chromosome 8 |
| | | | (RefSeq: NP_001105387.2) |
| | protein | | |
| 107 | CYCLIN-A1 | monocot | Sorghum bicolor |
| | (CYCA1) | | (RefSeq: XP_002455447.1) |
| | protein | | |
| 108 | CYCLIN-A1 | monocot | Sorghum bicolor |
| | (CYCA1) | | (RefSeq: XP_002454880.1) |
| | protein | | |
| 109 | CYCLIN-A1 | monocot | Sorghum bicolor |
| | (CYCA1) | | (RefSeq: XP_021311446.1) |
| | protein | | |
| 110 | CYCLIN-A1 | monocot | Oryza sativa |
| | (CYCA1) | | (RefSeq: XP_025879970.1) |
| | protein | | |
| 111 | CYCLIN-A1 | monocot | Oryza sativa |
| | (CYCA1) | | (RefSeq: XP_015630726.1) |
| | protein | | |
| 112 | CYCLIN-A1 | monocot | Oryza sativa |
| | (CYCA1) | | (RefSeq: XP_025878069.1) |
| | protein | | |
| 113 | CYCLIN-A1 | moBocol | Brachypodium distachyon |
| | (CYCA1) | | (RefSeq: XP_003566077.1) |
| | protein | | |
| 114 | CYCLIN-A1 | monocot | Brachypodium distachyon |
| | (CYCA1) | | (RefSeq: XP_003566081.1) |
| | protein | | |
| 115 | CYCLIN-A1 | monocot | Hordeum vulgare subsp. Vulgare |
| | (CYCA1) | | (RefSeq: XP_044975533.1) |
| | protein | | |
| 116 | CYCLIN-A1 | monocot | Triticum aestivum |
| | (CYCA1) | | (RefSeq: XP_044356181.1) |
| | protein | | |
| 117 | CYCLIN-A1 | monocot | Aegilops tauschii subsp. Strangulate |
| | (CYCA1) | | (RefSeq: XP_020187359.1) |
| | protein | | |
| 118 | CYCLIN-A1 | monocot | Muso acuminata subsp. Malaccensis |
| | (CYCA1) | | (RefSeq: XP_018678008.1) |
| | protein | | |
| 119 | CYCLIN-A1 | monocot | CYCA1 monocot |
| | (CYCA1) | | Alignment consensus |
| | protein | | sequence (Sequence Alignment 17) |
| 120 | PAIR1- | dicot | Solanum tuberosum |
| | targeting region | | |
| | of gRNA | | |
| 121 | PAIR1-targeting | dicot | Solanum tuberosum |
| | region of gRNA | | |
| 122 | SPO11-1- | dicot | Solanum tuberosum |
| | targeting region | | |
| | of gRNA | | |
| 123 | PAIR1-targeting | dicot | Solanum tuberosum |
| | region of gRNA | | |
| 124 | PAIR1-targeting | dicot | Solanum tuberosum |
| | region of gRNA | | |
| 125 | SPO11-1- | dicot | Solanum tuberosum |
| | targeting region | | |
| | of gRNA | | |
| 126 | PAIR1-targeting | dicot | Solanum tuberosum |
| | region of gRNA | | |
| 127 | SPO11-1- | dicot | Solanum tuberosum |
| | targeting region | | |
| | of gRNA | | |
| 128 | SPO11-1- | dicot | Solanum tuberosum |
| | targeting region | | |
| | of gRNA | | |
| 129 | SPO11-1- | dicot | Solanum tuberosum |
| | targeting region | | |
| | of gRNA | | |
| 130 | SPO11-1- | dicot | Solanum tuberosum |
| | targeting region | | |
| | of gRNA | | |
| 131 | SPO11-1- | dicot | Solanum tuberosum |
| | targeting region | | |
| | of gRNA | | |
| 132 | PAIR1-targeting | dicot | Solanum tuberosum |
| | region of gRNA | | |
| 133 | SPO11-1- | dicot | Solamon tuberosum |
| | targeting region | | |
| | of gRNA | | |
| 134 | SPO11-1- | dicot | Solanum tuberosum |
| | targeting region | | |
| | of gRNA | | |
| 135 | SPO11-1- | dicot | Solanum tuberosum |
| | targeting region | | |
| | of gRNA | | |
| 136 | REC8-targeting | dicot | Solanum tuberosum |
| | region of gRNA | | |
| 137 | REC8-targeting | dicot | Solanum tuberosum |
| | region of gRNA | | |
| 138 | REC8-targeting | dicot | Solanum tuberosum |
| | region of gRNA | | |
| 139 | REC8-targeting | dicot | Solanum tuberosum |
| | region of gRNA | | |
| 140 | PAIR1-targeting | dicot | Solanum tuberosum |
| | region of gRNA | | |
| 141 | REC8-targeting | dicot | Solanum tuberosum |
| | region of gRNA | | |
| 142 | PAIR1-targeting | dicot | Solanum tuberosum |
| | region of gRNA | | |
| 143 | SPO11-1- | dicot | Solanum tuberosum |
| | targeting region | | |
| | of gRNA | | |
| 144 | REC8-targeting | dicot | Solanum tuberosum |
| | region of gRNA | | |
| 145 | REC8-targeting | dicot | Solanum tuberosum |
| | region of gRNA | | |
| 146 | PAIR1-targeting | dicot | Solanum tuberosum |
| | region of gRNA | | |
| 147 | REC8-targeting | dicot | Solanum tuberosum |
| | region of gRNA | | |
| 148 | PAIR1-targeting | dicot | Solanum tuberosum |
| | region of gRNA | | |
| 149 | REC8-targeting | dicot | Solanum tuberosum |
| | region of gRNA | | |
| 150 | PAIR1-targeting | dicot | Solanum tuberosum |
| | region of gRNA | | |
| 151 | OSD1-targeting | dicot | Solanum tuberosum |
| | region of gRNA | | |
| 152 | REC8-targeting | dicot | Solanum tuberosum |
| | region of gRNA | | |
| 153 | OSD1-targeting | dicot | Solanum tuberosum |
| | region of gRNA | | |
| 154 | PAIR1-targeting | dicot | Solanum tuberosum |
| | region of gRNA | | |
| 155 | OSD1-targeting | dicot | Solanum tuberosum |
| | region of gRNA | | |
| 156 | OSD1-targeting | dicot | Solanum tuberosum |
| | region of gRNA | | |
| 157 | OSD1-targeting | dicot | Solanum tuberosum |
| | region of gRNA | | |
| 158 | PAIR1-targeting | dicot | Solanum tuberosum |
| | region of gRNA | | |
| 159 | OSD1-targeting | dicot | Solanum tuberosum |
| | region of gRNA | | |
| 160 | OSD1-targeting | dicot | Solanum tuberosum |
| | region of gRNA | | |
| 161 | SPO11-1- | dicot | Solanum tuberosum |
| | targeting region | | |
| | of gRNA | | |
| 162 | SPO11-1- | dicot | Solanum tuberosum |
| | targeting region | | |
| | of gRNA | | |
| 163 | SPO11-1- | dicot | Solanum tuberosum |
| | targeting region | | |
| | of gRNA | | |
| 164 | CYCA1- | dicot | Solanum tuberosum |
| | targeting region | | |
| | of gRNA | | |
| 165 | CYCA1- | dicot | Solanum tuberosum |
| | targeting region | | |
| | of gRNA | | |
| 166 | CYCA1- | dicot | Solanum tuberosum |
| | targeting region | | |
| | of gRNA | | |
| 167 | CYCA1- | dicot | Solanum tuberosum |
| | targeting region | | |
| | of gRNA | | |
| 168 | CYCA1- | dicot | Solanum tuberosum |
| | targeting region | | |
| | of gRNA | | |
| 169 | CYCA1- | dicot | Solanum tuberosum |
| | targeting region | | |
| | of gRNA | | |
| 170 | CYCA1- | dicot | Solanum tuberosum |
| | targeting region | | |
| | of gRNA | | |
| 171 | CYCA1- | dicot | Solanum tuberosum |
| | targeting region | | |
| | of gRNA | | |
| 172 | CYCA1- | dicot | Solanum tuberosum |
| | targeting region | | |
| | of gRNA | | |
| 173 | CYCA1- | dicot | Solanum tuberosum |
| | targeting region | | |
| | of gRNA | | |
| 174 | CYCA1- | dicot | Solanum tuberosum |
| | targeting region | | |
| | of gRNA | | |
| 175 | PAIR1 target | dicot | Solanum tuberosum |
| | sequence | | |
| 176 | PAIR1 target | dicot | Solanum tuberosum |
| | sequence | | |
| 177 | SPO11-1 target | dicot | Solanum tuberosum |
| | sequence | | |
| 178 | PAIR1 target | dicot | Solanum tuberosum |
| | sequence | | |
| 179 | PAIR1 target | dicot | Solanum tuberosum |
| | sequence | | |
| 180 | SPO11-1 target | dicot | Solanum tuberosum |
| | sequence | | |
| 181 | PAIR1 target | dicot | Solanum tuberosum |
| | sequence | | |
| 182 | SPO11-1 target | dicot | Solanum tuberosum |
| | sequence | | |
| 183 | SPO11-1 target | dicot | Solanum tuberosum |
| | sequence | | |
| 184 | SPO11-1 target | dicot | Solanum tuberosum |
| | sequence | | |
| 185 | SPO11-1 target | dicot | Solanum tuberosum |
| | sequence | | |
| 186 | SPO11-1 target | dicot | Solanum tuberosum |
| | sequence | | |
| 187 | PAIR1 target | dicot | Solanum tuberosum |
| | sequence | | |
| 188 | SPO11-1 target | dicot | Solanum tuberosum |
| | sequence | | |
| 189 | SPO11-1 target | dicot | Solanum tuberosum |
| | sequence | | |
| 190 | SPO11-1 target | dicot | Solanum tuberosum |
| | sequence | | |
| 191 | REC8 target | dicot | Solanum tuberosum |
| | sequence | | |
| 192 | REC8 target | dicot | Solanum tuberosum |
| | sequence | | |
| 193 | REC8 target | dicot | Solanum tuberosum |
| | sequence | | |
| 194 | REC8 target | dicot | Solanum tuberosum |
| | sequence | | |
| 195 | PAIR1 target | dicot | Solanum tuberosum |
| | sequence | | |
| 196 | REC8 target | dicot | Solanum tuberosum |
| | sequence | | |
| 197 | PAIR1 target | dicot | Solanum tuberosum |
| | sequence | | |
| 198 | SPO11-1 target | dicot | Solanum tuberosum |
| | sequence | | |
| 199 | REC8 target | dicot | Solanum tuberosum |
| | sequence | | |
| 200 | REC8 target | dicot | Solanum tuberosum |
| | sequence | | |
| 201 | PAIR1 target | dicot | Solanum tuberosum |
| | sequence | | |
| 202 | REC8 target | dicot | Solanum tuberosum |
| | sequence | | |
| 203 | PAIR1 target | dicot | Solanum tuberosum |
| | sequence | | |
| 204 | REC8 target | dicot | Solanum tuberosum |
| | sequence | | |
| 205 | PAIR1 target | dicot | Solanum tuberosum |
| | sequence | | |
| 206 | OSD1 target | dicot | Solanum tuberosum |
| | sequence | | |
| 207 | REC8 target | dicot | Solanum tuberosum |
| | sequence | | |
| 208 | OSD1 target | dicot | Solanum tuberosum |
| | sequence | | |
| 209 | PAIR1 target | dicot | Solanum tuberosum |
| | sequence | | |
| 210 | OSD1 target | dicot | Solanum tuberosum |
| | sequence | | |
| 211 | OSD1 target | dicot | Solanum tuberosum |
| | sequence | | |
| 212 | OSD1 target | dicot | Solanum tuberosum |
| | sequence | | |
| 213 | PAIR1 target | dicot | Solanum tuberosum |
| | sequence | | |
| 214 | OSD1 target | dicot | Solanum tuberosum |
| | sequence | | |
| 215 | OSD1 target | dicot | Solanum tuberosum |
| | sequence | | |
| 216 | SPO11-1 target | dicot | Solanum tuberosum |
| | sequence | | |
| 217 | SPO11-1 target | dicot | Solanum tuberosum |
| | sequence | | |
| 218 | SPO11-1 target | dicot | Solanum tuberosum |
| | sequence | | |
| 219 | CYCA1 target | dicot | Solanum tuberosum |
| | sequence | | |
| 220 | CYCA1 target | dicot | Solanum tuberosum |
| | sequence | | |
| 221 | CYCA1 target | dicot | Solanum tuberosum |
| | sequence | | |
| 222 | CYCA1 target | dicot | Solanum tuberosum |
| | sequence | | |
| 223 | CYCA1 target | dicot | Solanum tuberosum |
| | sequence | | |
| 224 | CYCA1 target | dicot | Solanum tuberosum |
| | sequence | | |
| 225 | CYCA1 target | dicot | Solanum tuberosum |
| | sequence | | |
| 226 | CYCA1 target | dicot | Solanum tuberosum |
| | sequence | | |
| 227 | CYCA1 target | dicot | Solanum tuberosum |
| | sequence | | |
| 228 | CYCA1 target | dicot | Solanum tuberosum |
| | sequence | | |
| 229 | CYCA1 target | dicot | Solanum tuberosum |
| | sequence | | |
| 230 | OSD1-1 target | monocot | Zea mays |
| | sequence | | |
| 231 | OSD1-1 target | monocot | Zea mays |
| | sequence | | |
| 232 | OSD1-1 target | monocot | Zea mays |
| | sequence | | |
| 233 | OSD1-1 target | monocot | Zea mays |
| | sequence | | |
| 234 | OSD1-1 target | monocot | Zea mays |
| | sequence | | |
| 235 | OSD1-1 target | monocot | Zea mays |
| | sequence | | |
| 236 | OSD1-1 target | monocot | Zea mays |
| | sequence | | |
| 237 | OSD1-1 target | monocot | Zea mays |
| | sequence | | |
| 238 | OSD1-1 target | monocot | Zea mays |
| | sequence | | |
| 239 | OSD1-1 target | monocot | Zea mays |
| | sequence | | |
| 240 | OSD1-2 target | monocot | Zea mays |
| | sequence | | |
| 241 | OSD1-2 target | monocot | Zea mays |
| | sequence | | |
| 242 | OSD1-2 target | monocot | Zea mays |
| | sequence | | |
| 243 | OSD1-2 target | monocot | Zea mays |
| | sequence | | |
| 244 | OSD1-2 target | monocot | Zea mays |
| | sequence | | |
| 245 | OSD1-2 target | monocot | Zea mays |
| | sequence | | |
| 246 | OSD1-2 target | monocot | Zea mays |
| | sequence | | |
| 247 | OSD1-2 target | monocot | Zea mays |
| | sequence | | |
| 248 | OSD1-2 target | monocot | Zea mays |
| | sequence | | |
| 249 | REC8 target | monocot | Zea mays |
| | sequence | | |
| 250 | REC8 target | monocot | Zea mays |
| | sequence | | |
| 251 | REC8 target | monocot | Zea mays |
| | sequence | | |
| 252 | REC8 target | monocot | Zea mays |
| | sequence | | |
| 253 | REC8 target | monocot | Zea mays |
| | sequence | | |
| 254 | REC8 target | monocot | Zea mays |
| | sequence | | |
| 255 | REC8 target | monocot | Zea mays |
| | sequence | | |
| 256 | REC8 target | monocot | Zea mays |
| | sequence | | |
| 257 | REC8 target | monocot | Zea mays |
| | sequence | | |
| 258 | SPO11-1 target | monocot | Zea mays |
| | sequence | | |
| 259 | SPO11-1 target | monocot | Zea mays |
| | sequence | | |
| 260 | SPO11-1 target | monocot | Zea mays |
| | sequence | | |
| 261 | SPO11-1 target | monocot | Zea mays |
| | sequence | | |
| 262 | SPO11-1 target | monocot | Zea mays |
| | sequence | | |
| 263 | SPO11-1 target | monocot | Zea mays |
| | sequence | | |
| 264 | SPO11-1 target | monocot | Zea mays |
| | sequence | | |
| 265 | SPO11-1 target | monocot | Zea mays |
| | sequence | | |
| 266 | SPO11-1 target | monocot | Zea mays |
| | sequence | | |
| 267 | SPO11-1 target | monocot | Zea mays |
| | sequence | | |
| 268 | SPO11-1 target | monocot | Zea mays |
| | sequence | | |
| 269 | SPO11-1 target | monocot | Zea mays |
| | sequence | | |
| 270 | SPO11-1 target | monocot | Zea mays |
| | sequence | | |
| 271 | SPO11-1 target | monocot | Zea mays |
| | sequence | | |
| 272 | SPO11-1 target | monocot | Zea mays |
| | sequence | | |
| 273 | SPO11-1 target | monocot | Zea mays |
| | sequence | | |
| 274 | SPO11-1 target | monocot | Zea mays |
| | sequence | | |
| 275 | SPO11-1 target | monocot | Zea mays |
| | sequence | | |
| 276 | SPO11-1 target | monocot | Zea mays |
| | sequence | | |
| 277 | OSD1-1- | monocot | Zea mays |
| | targeting region | | |
| | of gRNA | | |
| 278 | OSD1-1- | monocot | Zea mays |
| | targeting region | | |
| | of gRNA | | |
| 279 | OSD1-1- | monocot | Zea mays |
| | targeting region | | |
| | of gRNA | | |
| 280 | OSD1-1- | monocot | Zea mays |
| | targeting region | | |
| | of gRNA | | |
| 281 | OSD1-1- | monocot | Zea mays |
| | targeting region | | |
| | of gRNA | | |
| 282 | OSD1-1- | monocot | Zea mays |
| | targeting region | | |
| | of gRNA | | |
| 283 | OSD1-1- | monocot | Zea mays |
| | targeting region | | |
| | of gRNA | | |
| 284 | OSD1-1- | monocot | Zea mays |
| | targeting region | | |
| | of gRNA | | |
| 285 | OSD1-1- | monocot | Zea mays |
| | targeting region | | |
| | of gRNA | | |
| 286 | OSD1-1- | monocot | Zea mays |
| | targeting region | | |
| | of gRNA | | |
| 287 | OSD1-2- | monocot | Zea mays |
| | targeting region | | |
| | of gRNA | | |
| 288 | OSD1-2- | monocot | Zea mays |
| | targeting region | | |
| | of gRNA | | |
| 289 | OSD1-2- | monocot | Zea mays |
| | targeting region | | |
| | of gRNA | | |
| 290 | OSD1-2- | monocot | Zea mays |
| | targeting region | | |
| | of gRNA | | |
| 291 | OSD1-2- | monocot | Zea mays |
| | targeting region | | |
| | of gRNA | | |
| 292 | OSD1-2- | monocot | Zea mays |
| | targeting region | | |
| | of gRNA | | |
| 293 | OSD1-2- | monocot | Zea mays |
| | targeting region | | |
| | of gRNA | | |
| 294 | OSD1-2- | monocot | Zea mays |
| | targeting region | | |
| | of gRNA | | |
| 295 | OSD1-2- | monocot | Zea mays |
| | targeting region | | |
| | of gRNA | | |
| 296 | REC8-targeting | monocot | Zea mays |
| | region of gRNA | | |
| 297 | REC8-targeting | monocot | Zea mays |
| | region of gRNA | | |
| 298 | REC8-targeting | monocot | Zea mays |
| | region of gRNA | | |
| 299 | REC8-targeting | monocot | Zea mays |
| | region of gRNA | | |
| 300 | REC8-targeting | monocot | Zea mays |
| | region of gRNA | | |
| 301 | REC8-targeting | monocot | Zea mays |
| | region of gRNA | | |
| 302 | REC8-targeting | monocot | Zea mays |
| | region of gRNA | | |
| 303 | REC8-targeting | monocot | Zea mays |
| | region of gRNA | | |
| 304 | REC8-targeting | monocot | Zea mays |
| | region of gRNA | | |
| 305 | SPO11-1- | monocot | Zea mays |
| | targeting region | | |
| | of gRNA | | |
| 306 | SPO11-1- | monocot | Zea mays |
| | targeting region | | |
| | of gRNA | | |
| 307 | SPO11-1- | monocot | Zea mays |
| | targeting region | | |
| | of gRNA | | |
| 308 | SPO11-1- | monocot | Zea mays |
| | targeting region | | |
| | of gRNA | | |
| 309 | SPO11-1- | monocot | Zea mays |
| | targeting region | | |
| | of gRNA | | |
| 310 | SPO11-1- | monocot | Zea mays |
| | targeting region | | |
| | of gRNA | | |
| 311 | SPO11-1- | monocot | Zea mays |
| | targeting region | | |
| | of gRNA | | |
| 312 | SPO11-1- | monocot | Zea mays |
| | targeting region | | |
| | of gRNA | | |
| 313 | SPO11-1- | monocot | Zea mays |
| | targeting region | | |
| | of gRNA | | |
| 314 | SPO11-1- | monocot | Zea mays |
| | targeting region | | |
| | of gRNA | | |
| 315 | SPO11-1- | monocot | Zea mays |
| | targeting region | | |
| | of gRNA | | |
| 316 | SPO11-1- | monocot | Zea mays |
| | targeting region | | |
| | of gRNA | | |
| 317 | SPO11-1- | monocot | Zea mays |
| | targeting region | | |
| | of gRNA | | |
| 318 | SPO11-1- | monocot | Zea mays |
| | targeting region | | |
| | of gRNA | | |
| 319 | SPO11-1- | monocot | Zea mays |
| | targeting region | | |
| | of gRNA | | |
| 320 | SPO11-1- | monocot | Zea mays |
| | targeting region | | |
| | of gRNA | | |
| 321 | SPO11-1- | monocot | Zea mays |
| | targeting region | | |
| | of gRNA | | |
| 322 | SPO11-1- | monocot | Zea mays |
| | targeting region | | |
| | of gRNA | | |
| 323 | SPO11-1- | monocot | Zea mays |
| | targeting region | | |
| | of gRNA | | |
| 324 | OSD1-targeting | dicot | A. thaliana |
| | region of gRNA | | |
| 325 | OSD1-targeting | dicot | A. thaliana |
| | region of gRNA | | |
| 326 | OSD1-targeting | dicot | A. thaliana |
| | region of gRNA | | |
| 327 | OSD1-targeting | dicot | A. thaliana |
| | region of gRNA | | |
| 328 | OSD1-targeting | dicot | A. thaliano |
| | region of gRNA | | |
| 329 | OSD1-targeting | dicot | A. thaliana |
| | region of gRNA | | |
| 330 | OSD1-targeting | dicot | A. thaliana |
| | region of gRNA | | |
| 331 | OSD1-targeting | dicot | A. thaliana |
| | region of gRNA | | |
| 332 | OSD1-targeting | dicot | A. thaliana |
| | region of gRNA | | |
| 333 | OSD1-targeting | dicot | A. thaliana |
| | region of gRNA | | |
| 334 | OSD1-targeting | dicot | A. thaliana |
| | region of gRNA | | |
| 335 | OSD1-targeting | dicot | A. thaliana |
| | region of gRNA | | |
| 336 | PAIR1-targeting | dicot | A. thaliana |
| | region of gRNA | | |
| 337 | PAIR1-targeting | dicot | A. thaliana |
| | region of gRNA | | |
| 338 | PAIR1-targeting | dicot | A. thaliana |
| | region of gRNA | | |
| 339 | PAIR1-targeting | dicot | A. thaliana |
| | region of gRNA | | |
| 340 | PAIR1-targeting | dicot | A. thaliana |
| | region of gRNA | | |
| 341 | PAIR1-targeting | dicot | A. thaliana |
| | region of gRNA | | |
| 342 | PAIR1-targeting | dicot | A. thaliana |
| | region of gRNA | | |
| 343 | PAIR1-targeting | dicot | A. thaliana |
| | region of gRNA | | |
| 344 | PAIR1-targeting | dicot | A. thaliana |
| | region of gRNA | | |
| 345 | PAIR1-targeting | dicot | A. thaliana |
| | region of gRNA | | |
| 346 | PAIR1-targeting | dicot | A. thaliana |
| | region of gRNA | | |
| 347 | PAIR1-targeting | dicot | A. thaliana |
| | region of gRNA | | |
| 348 | PAIR1-targeting | dicot | A. thaliana |
| | region of gRNA | | |
| 349 | PAIR1-targeting | dicot | A. thaliana |
| | region of gRNA | | |
| 350 | PAIR1-targeting | dicot | A. thaliana |
| | region of gRNA | | |
| 351 | PAIR1-targeting | dicot | A. thaliana |
| | region of gRNA | | |
| 352 | PAIR1-targeting | dicot | A. thaliana |
| | region of gRNA | | |
| 353 | REC8-targeting | dicot | A. thaliana |
| | region of gRNA | | |
| 354 | REC8-targeting | dicot | A. thaliana |
| | region of gRNA | | |
| 355 | REC8-targeting | dicot | A. thaliana |
| | region of gRNA | | |
| 356 | REC8-targeting | dicot | A. thaliana |
| | region of gRNA | | |
| 357 | REC8-targeting | dicot | A. thaliana |
| | region of gRNA | | |
| 358 | REC8-targeting | dicot | A. thaliana |
| | region of gRNA | | |
| 359 | REC8-targeting | dicot | A. thaliana |
| | region of gRNA | | |
| 360 | REC8-targeting | dicot | A. thaliana |
| | region of gRNA | | |
| 361 | REC8-targeting | dicot | A. thaliana |
| | region of gRNA | | |
| 362 | REC8-targeting | dicot | A. thaliana |
| | region of gRNA | | |
| 363 | REC8-targeting | dicot | A. thaliana |
| | region of gRNA | | |
| 364 | REC8-targeting | dicot | A. thaliana |
| | region of gRNA | | |
| 365 | REC8-targeting | dicot | A. thaliana |
| | region of gRNA | | |
| 366 | REC8-targeting | dicot | A. thaliana |
| | region of gRNA | | |
| 367 | REC8-targeting | dicot | A. thaliana |
| | region of gRNA | | |
| 368 | REC8-targeting | dicot | A. thaliana |
| | region of gRNA | | |
| 369 | REC8-targeting | dicot | A. thaliana |
| | region of gRNA | | |
| 370 | REC8-targeting | dicot | A. thaliana |
| | region of gRNA | | |
| 371 | REC8-targeting | dicot | A. thaliana |
| | region of gRNA | | |
| 372 | SPO11-1- | dicot | A. thaliana |
| | targeting region | | |
| | of gRNA | | |
| 373 | SPO11-1- | dicot | A. thaliana |
| | targeting region | | |
| | of gRNA | | |
| 374 | SPO11-1- | dicot | A. thaliana |
| | targeting region | | |
| | of gRNA | | |
| 375 | SPO11-1- | dicot | A. thaliana |
| | targeting region | | |
| | of gRNA | | |
| 376 | SPO11-1- | dicot | A. thaliana |
| | targeting region | | |
| | of gRNA | | |
| 377 | SPO11-1- | dicot | A. thaliana |
| | targeting region | | |
| | of gRNA | | |
| 378 | SPO11-1- | dicot | A. thaliana |
| | targeting region | | |
| | of gRNA | | |
| 379 | SPO11-1- | dicot | A. thaliana |
| | targeting region | | |
| | of gRNA | | |
| 380 | SPO11-1- | dicot | A. thaliana |
| | targeting region | | |
| | of gRNA | | |
| 381 | SPO11-1- | dicot | A. thaliana |
| | targeting region | | |
| | of gRNA | | |
| 382 | SPO11-1- | dicot | A. thaliana |
| | targeting region | | |
| | of gRNA | | |
| 383 | SPO11-1- | dicot | A. thaliana |
| | targeting region | | |
| | of gRNA | | |
| 384 | SPO11-1- | dicot | A. thaliana |
| | targeting region | | |
| | of gRNA | | |
| 385 | SPO11-1- | dicot | A. thaliana |
| | targeting region | | |
| | of gRNA | | |
| 386 | OSD1 target | dicot | A. thaliana |
| | sequence | | |
| 387 | OSD1 target | dicot | A. thaliana |
| | sequence | | |
| 388 | OSD1 target | dicot | A. thaliana |
| | sequence | | |
| 389 | OSD1 target | dicot | A. thaliana |
| | sequence | | |
| 390 | OSD1 target | dicot | A. thaliana |
| | sequence | | |
| 391 | OSD1 target | dicot | A. thaliana |
| | sequence | | |
| 392 | OSD1 target | dicot | A. thaliana |
| | sequence | | |
| 393 | OSD1 target | dicot | A. thaliana |
| | sequence | | |
| 394 | OSD1 target | dicot | A. thaliana |
| | sequence | | |
| 395 | OSD1 target | dicot | A. thaliana |
| | sequence | | |
| 396 | OSD1 target | dicot | A. thaliana |
| | sequence | | |
| 397 | OSD1 target | dicot | A. thaliana |
| | sequence | | |
| 398 | PAIR1 target | dicot | A. thaliana |
| | sequence | | |
| 399 | PAIR1 target | dicot | A. thaliana |
| | sequence | | |
| 400 | PAIR1 target | dicot | A. thaliana |
| | sequence | | |
| 401 | PAIR1 target | dicot | A. thaliana |
| | sequence | | |
| 402 | PAIR1 target | dicot | A. thaliana |
| | sequence | | |
| 403 | PAIR1 target | dicot | A. thaliana |
| | sequence | | |
| 404 | PAIR1 target | dicot | A. thaliana |
| | sequence | | |
| 405 | PAIR1 target | dicot | A. thaliana |
| | sequence | | |
| 406 | PAIR1 target | dicot | A. thaliana |
| | sequence | | |
| 407 | PAIR1 target | dicot | A. thaliana |
| | sequence | | |
| 408 | PAIR1 target | dicot | A. thaliana |
| | sequence | | |
| 409 | PAIR1 target | dicot | A. thaliana |
| | sequence | | |
| 410 | PAIR1 target | dicot | A. thaliana |
| | sequence | | |
| 411 | PAIR1 target | dicot | A. thaliana |
| | sequence | | |
| 412 | PAIR1 target | dicot | A. thaliana |
| | sequence | | |
| 413 | PAIR1 target | dicot | A. thaliana |
| | sequence | | |
| 414 | PAIR1 target | dicot | A. thaliana |
| | sequence | | |
| 415 | REC8 target | dicot | A. thaliana |
| | sequence | | |
| 416 | REC8 target | dicot | A. thaliana |
| | sequence | | |
| 417 | REC8 target | dicot | A. thaliana |
| | sequence | | |
| 418 | REC8 target | dicot | A. thaliana |
| | sequence | | |
| 419 | REC8 target | dicot | A. thaliana |
| | sequence | | |
| 420 | REC8 target | dicot | A. thaliana |
| | sequence | | |
| 421 | REC8 target | dicot | A. thaliana |
| | sequence | | |
| 422 | REC8 target | dicot | A. thaliana |
| | sequence | | |
| 423 | REC8 target | dicot | A. thaliana |
| | sequence | | |
| 424 | REC8 target | dicot | A. thaliana |
| | sequence | | |
| 425 | REC8 target | dicot | A. thaliana |
| | sequence | | |
| 426 | REC8 target | dicot | A. thaliana |
| | sequence | | |
| 427 | REC8 target | dicot | A. thaliana |
| | sequence | | |
| 428 | REC8 target | dicot | A. thaliana |
| | sequence | | |
| 429 | REC8 target | dicot | A. thaliana |
| | sequence | | |
| 430 | REC8 target | dicot | A. thaliana |
| | sequence | | |
| 431 | REC8 target | dicot | A. thaliana |
| | sequence | | |
| 432 | REC8 target | dicot | A. thaliana |
| | sequence | | |
| 433 | REC8 target | dicot | A. thaliana |
| | sequence | | |
| 434 | SPO11-1 target | dicot | A. thaliana |
| | sequence | | |
| 435 | SPO11-1 target | dicot | A. thaliana |
| | sequence | | |
| 436 | SPO11-1 target | dicot | A. thaliana |
| | sequence | | |
| 437 | SPO11-1 target | dicot | A. thaliana |
| | sequence | | |
| 438 | SPO11-1 target | dicot | A. thaliana |
| | sequence | | |
| 439 | SPO11-1 target | dicot | A. thaliana |
| | sequence | | |
| 440 | SPO11-1 target | dicot | A. thaliana |
| | sequence | | |
| 441 | SPO11-1 target | dicot | A. thaliana |
| | sequence | | |
| 442 | SPO11-1 target | dicot | A. thaliana |
| | sequence | | |
| 443 | SPO11-1 target | dicot | A. thaliana |
| | sequence | | |
| 444 | SPO11-1 target | dicot | A. thaliana |
| | sequence | | |
| 445 | SPO11-1 target | dicot | A. thaliana |
| | sequence | | |
| 446 | SPO11-1 target | dicot | A. thaliana |
| | sequence | | |
| 447 | SPO11-1 target | dicot | A. thaliana |
| | sequence | | |
| 448 | osd1-2 allele | monocot | Zea mays, PED-MN-MiMe |
| 449 | osd1-2 | monocot | Zea mays, PED-MN-MiMe |
| | allele | | |
| 450 | rec8 allele | monocot | Zea mays, PED-MN-MiMe |
| 451 | spo11 allele | monocot | Zea mays, PED-MN-MiMe |
| 452 | osd1 allele | dicot | Arabidopsis thaliana PED-AR-AA |
| 453 | osd1 allele | dicot | Arabidopsis thaliana PED-AR-AA |
| 454 | rec8 allele | dicot | Arabidopsis thaliana PED-AR-AA |
| 455 | rec8 allele | dicot | Arabidopsis thaliana PED-AR-AA |
| 456 | spo11 allele | dicot | Arabidopsis thaliana PED-AR-AA |
| 457 | spo11 allele | dicot | Arabidopsis thaliana PED-AR-AA |
| 458 | osd1 allele | dicot | Arabidopsis thaliana PED-AR-BC |
| 459 | osd1: xec8 allele | dicot | Arabidopsis thaliana PED-AR-BC |
| | (trans-location) | | |
| 460 | rec8 allele | dicot | Arabidopsis thaliana PED-AR-BC |
| 461 | pair1: trec8 allele | dicot | Arabidopsis thaliana PED-AR-BC |
| | (trans-location) | | |
| 462 | pair1 allele | dicot | Arabidopsis thaliana PED-AR-BC |
| 463 | pair1: osd1 | dicot | Arabidopsis thaliana PED-AR-BC |
| | allele | | |
| | (trans-location) | | |
| 464 | spo11 allele | dicot | Solanum tuberosum |
| | | | PED-PR-AB-sp |
| 465 | spo11: rec8 | dicot | Solanum tuberosum |
| | allele | | PED-PR-AB-sp |
| | (trans-location) | | |
| 466 | rec8: spo11 | dicot | Solanum tuberosum |
| | allele | | PED-PR-AB-sp |
| | (trans-location) | | |
| 468 | rec8 allele | dicot | Solanum tuberosum |
| | | | PED-PR-EF-rso-1 |
| 469 | rec8 allele | dicot | Solanum tuberosum |
| | | | PED-PR-EF-rso-1 |
| 470 | spo11 allele | dicot | Solanum tuberosum |
| | | | PED-PR-EF-rso-1 |
| 471 | spo11 allele | dicot | Solanum tuberosum |
| | | | PED-PR-EF-rso-1 |
| 474 | rec8 allele | dicot | Solanum tuberosum |
| | | | PED-PR-AB-rso |
| 475 | rec8 allele | dicot | Solanum tuberosum |
| | | | PED-PR-AB-rso |
| 476 | spo11 allele | dicot | Solanum tuberosum |
| | | | PED-PR-AB-rso |
| 477 | spo11 allele | dicot | Solanum tuberosum |
| | | | PED-PR-AB-rso |
| 478 | spo11 allele | dicot | Solanum tuberosum |
| | | | PED-PR-AD-sp |
| 479 | spo11 allele | dicot | Solanum tuberosum |
| | | | PED-PR-AD-sp |
| 480 | tam (cyca1) | dicot | Solanum tuberosum |
| | allele | | PED-PR-EF-rcs |
| 481 | tam (cyca1) | dicot | Solanum tuberosum |
| | allele | | PED-PR-EF-rcs |
| 482 | rec8 allele | dicot | Solanum tuberosum |
| | | | PED-PR-EF-rcs |
| 483 | rec8 allele | dicot | Solanum tuberosum |
| | | | PED-PR-EF-rcs |
| 484 | spo11 allele | dicot | Solanum tuberosum |
| | | | PED-PR-EF-rcs |
| 485 | spo11 allele | dicot | Solanum tuberosum |
| | | | PED-PR-EF-rcs |
| 486 | tam (cyca1) | dicot | Solanum tuberosum |
| | allele | | PED-PR-AB-rcs |
| 487 | tam (cyca1) | dicot | Solanum tuberosum |
| | allele | | PED-PR-AB-rcs |
| 488 | rec8 allele | dicot | Solanum tuberosum |
| | | | PED-PR-AB-rcs |
| 489 | rec8 allele | dicot | Solanum tuberosum |
| | | | PED-PR-AB-rcs |
| 490 | spo11 allele | dicot | Solanum tuberosum |
| | | | PED-PR-AB-rcs |
| 491 | spo11 allele | dicot | Solanum tuberosum |
| | | | PED-PR-AB-rcs |
| 493 | rec8 allele | dicot | Solanum tuberosum |
| | | | PED-PR-EF-rso-2 |
| 494 | rec8 allele | dicot | Solanum tuberosum |
| | | | PED-PR-EF-rso-2 |
| 495 | spo11 allele | dicot | Solanum tuberosum: |
| | | | PED-PR-EF-rso-2 |
| 496 | spo11 allele | dicot | Solanum tuberosum |
| | | | PED-PR-EF-rso-2 |

TABLE 10
Exemplary combinations of in MiMe alleles of parent
MiMe plants having a complete MiMe genotype.

| Genotype | Complete MiMe Genotype |
|---|---|
| 1 | rec8, spo11-1, cyca1 |
| 2 | rec8, pair1, cyca1 |
| 3 | rec8, pair1, spo11-1, cyca1 |
| 4 | rec8, spo11-1, TDM1* |
| 5 | rec8, pair1, TDM1* |
| 6 | rec8, pair1, spo11-1, TDM1* |
| 7 | rec8, spo11-1, TDM1*, cyca1 |
| 8 | rec8, pair1, TDM1*, cyca1 |
| 9 | rec8, pair1, spo11-1, TDM1*, cyca1 |
| 10 | spo11-1, ps1 |
| 10 | spo11-1, ps1, ps1-like |
| 11 | spo11-1, ps1-like |
| 12 | spo11-1, ps1-like, jason |
| 13 | spo11-1, jason |
| 14 | spo11-1, ps1, ps1-like, jason |
| 15 | pair1, ps1 |
| 16 | pair1, spo11-1, ps1 |
| 17 | spo11-1, ps1, ps1-like |
| 18 | pair1, spo11-1, ps1, ps1-like |
| 19 | pair1, ps1-like |
| 20 | pair1, spo11-1, ps1-like |
| 21 | pair1, ps1-like, jason |
| 22 | pair1, spo11-1, ps1-like, jason |
| 23 | pair1, jason |
| 24 | pair1, spo11-1, jason |
| 25 | pair1, ps1, ps1-like, jason |
| 26 | pair1, spo11-1, ps1, ps1-like, jason |
| 27 | rec8, spo11-1, osd1 |
| 28 | rec8, pair1, osd1 |
| 29 | rec8, pair1, spo11-1, osd1 |
| 30 | rec8, spo11-1, os |
| 31 | rec8, pair1, os |
| 32 | rec8, pair1, spo11-1, os |
| 33 | spo11-1, ps |
| 34 | spo11-1, ps, ps1 |
| 35 | spo11-1, ps, ps1, ps1-like |
| 36 | spo11-1, ps, ps1-like |
| 37 | spo11-1, ps, jason |
| 38 | spo11-1, ps, ps1, jason |
| 39 | spo11-1, ps, ps1-like, jason |
| 40 | spo11-1, ps, ps1, ps1-like, jason |
| 41 | pair1, ps |
| 42 | pair1, ps, ps1 |
| 43 | pair1, ps, ps1, ps1-like |
| 44 | pair1, ps, ps1-like |
| 45 | pair1, ps, jason |
| 46 | pair1, ps, ps1, jason |
| 47 | pair1, ps, ps1-like, jason |
| 48 | pair1, ps, ps1, ps1-like, jason |
| 49 | pair1, spo11-1, ps |
| 50 | pair1, spo11-1, ps, ps1 |
| 51 | pair1, spo11-1, ps, ps1, ps1-like |
| 52 | pair1, spo11-1, ps, ps1-like |
| 53 | pair1, spo11-1, ps, jason |
| 54 | pair1, spo11-1, ps, ps1, jason |
| 55 | pair1, spo11-1, ps, ps1-like, jason |
| 56 | pair1, spo11-1, ps, ps1, ps1-like, jason |

*Dominant mutation

### ENUMERATED EMBODIMENTS

The following enumerated embodiments are representative of some aspects of the invention.
1. A population of polyploid seed comprising a subpopulation of genetically uniform polyploid seed in an amount of at least 50% of the total number of seeds, the genetically uniform polyploid seed comprising three or more haplotypes of the same or related species of plant, wherein the population was obtained from a single plant or a set of F1 hybrids.
2. The population of embodiment 1, wherein the subpopulation of genetically uniform polyploid seed is triploid, tetraploid, pentaploid, hexaploid, heptaploid, or octoploid.
3. The population of embodiment 1 or 2, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.
4. The population of any one of embodiments 1-3, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.
5. The population of embodiment 1-4, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.
6. The population of any one of embodiments 1-5, wherein the subpopulation of genetically uniform polyploid seed has a complete MiMe genotype comprising:
   (A)
   MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
   (B)
   MiMe alleles conferring decreased expression of one or more Mime loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components.
7. The population of any one of embodiments 1-5, wherein the subpopulation of genetically uniform polyploid seed has a partial MiMe genotype comprising:
   (A)
      (a) one or more MiMe alleles conferring decreased expression of one or more MiMe loci of each of a first, second, and third MiMe component; and
      (b) one or more non-MiMe alleles at the one or more MiMe loci of each of the first, second, and third MiMe component,
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
   (B)
      (a) one or more MiMe alleles conferring decreased expression of one or more Mime loci of each of the first and second MiMe component; and
      (b) one or more non-MiMe alleles at the one or more MiMe loci of each of the first and second MiMe component,
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.
8. The population of any one of embodiments 1-5, wherein the subpopulation of genetically uniform polyploid seed has a partially-complemented MiMe genotype comprising:
   (A)
      (a) only MiMe alleles at one or more MiMe loci of a first Mime component;
      (b) one or more Mime alleles and one or more non-MiMe alleles at a first Mime locus of a second MiMe component, and one or more MiMe alleles and one or more non-MiMe alleles at a second MiMe locus of the second MiMe component; and
      (c) either (i) only Mime alleles at one or more MiMe loci of a third Mime component, or (ii) one or more Mime alleles and one or more non-MiMe alleles at a first Mime locus of the third MiMe component, and one or more MiMe alleles and one or more non-MiMe alleles at a second MiMe locus of the third MiMe component,
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
   (B)
      (a) only MiMe alleles at one or more MiMe loci of a first Mime component; and
      (b) one or more Mime alleles and one or more non-MiMe alleles at a first Mime locus of a second MiMe component, and one or more MiMe alleles and one or more non-MiMe alleles at a second MiMe locus of the second MiMe component,
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; or
   (C)
      (a) only MiMe alleles at one or more MiMe loci of a first Mime component, wherein the first Mime component is a component of DNA double strand breakage during meiotic recombination;
      (b) one or more Mime alleles and one or more non-MiMe alleles at one or more Mime loci of a second MiMe component;
      (c) one or more MiMe alleles and one or more non-MiMe alleles at one or more MiMe loci of a third MiMe component; and
      (d) one or more Mime alleles and one or more non-MiMe alleles at one or more Mime loci of a fourth MiMe component,
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
9. The population of any one of embodiments 6-8, wherein:
   (1) the one or more MiMe loci of the component of sister chromatid cohesion during the first division of meiosis comprise REC8, SWITCH1/DYAD, or a combination thereof;
   (2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
   (3) the one or more MiMe loci of the component of progression through the second division of meiosis comprise OSD1, CYCA1, TDM1, PC1, PC2, FC, or any combination thereof; and/or
   (4) the one or more MiMe loci of the component of progression through the first division of meiosis comprise PS1, JASON, or a combination thereof.
10. The population of any one of embodiments 1-9, wherein the subpopulation of genetically uniform polyploid seed comprises:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the subpopulation of genetically uniforms seed is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the subpopulation of genetically uniforms seed is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO111-1;
   (15) a complete MiMe genotype comprising (i) a ps allele, wherein the subpopulation of genetically uniforms seed is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the subpopulation of genetically uniforms seed is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (20) a partially complemented MiMe genotype comprising (i) a ps allele, wherein the subpopulation of genetically uniforms seed is heterozygous for the ps allele, (ii) an os allele, wherein the subpopulation of genetically uniforms seed is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the subpopulation of genetically uniforms seed is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
11. The population of any one of embodiments 1-9, wherein the subpopulation of genetically uniform polyploid seed comprises one or more polynucleotide sequences selected from the group consisting of SEQ ID NOs: 448-466, 468-471, 474-491, and 493-496.
12. The population of any one of embodiments 1-11, wherein germination of a seed of the subpopulation of genetically uniform polyploid seed results in a sterile plant that produces inviable gametes, seedless fruit, or a combination thereof.
13. The population of any one of embodiments 1-12, wherein the population of polyploid seed is from a plant selected from the group consisting of potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussel sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine, mandarin, clementine, coffee, cola, hazelnut, saffron, melon, cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycrass, green bean, bean, ground cherry, pine, pistachio nut, pea, turf grass, poplar, apricot, plum, prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.
14. A method of producing the population of polyploid seed of any one of embodiments 1-13, the method comprising:
   (a) generating a first parent MiMe plant and a second parent MiMe plant by introducing genetic modifications into one or more candidate lines to produce MiMe alleles in germplasm of a first parent MiMe plant, a second parent MiMe plant, and/or progenitors thereof, wherein the first parent Mime plant and the second parent MiMe plant each comprise a complete MiMe genotype, and each of the progenitors comprises a partial MiMe genotype, wherein if progenitors are generated, the progenitors are further crossed to generate the first parent MiMe plant, the second parent MiMe plant, or both;
   (b) providing clonal gametes from the first parent MiMe plant and the second parent MiMe plant that together comprise the three or more haplotypes; and
   (c) crossing the clonal gametes to produce the population of polyploid seed; wherein the population of polyploid seed comprises a subpopulation of genetically uniform polyploid seed in an amount of at least 50% of the total number of seeds, the subpopulation of genetically uniform polyploid seed comprising the three or more haplotypes.
15. The method of embodiment 14, wherein the subpopulation of genetically uniform polyploid seed has a partially-complemented MiMe genotype, wherein:
   (A)
      (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
      (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
   (B)
      (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
      (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
   (C)
      (a) the first parent MiMe plant has only MiMe alleles at one or more Mime loci of a first Mime component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
      (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
      wherein at least one of the Mime loci having only Mime alleles of the first MiMe component of the first parent Mime plant is the same as at least one of the Mime loci having only MiMe alleles of the first MiMe component of the second parent MiMe plant;
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
16. A method of producing the population of polyploid seed of any one of embodiments 1-13, the method comprising:
   (a) generating a first parent MiMe plant by introducing genetic modifications into one or more candidate lines to produce MiMe alleles in germplasm of the first parent MiMe plant or progenitors thereof, wherein the first parent Mime plant comprises a complete MiMe genotype, and each of the progenitors comprises a partial MiMe genotype, wherein if progenitors are generated, the progenitors are further crossed to generate the first parent Mime plant;
   (b) providing clonal gametes from the first parent Mime plant;
   (c) providing haploid gametes from a homozygous parent non-MiMe plant; and
   (d) crossing the clonal gametes with the haploid gametes to produce the population of polyploid seed, wherein the clonal gametes and the haploid gametes together comprise three or more haplotypes, optionally wherein step (a) comprises generating the first parent MiMe plant, the second parent MiMe plant, or both.
17. A method of breeding a polyploid hybrid plant line, the method comprising:
   (a) obtaining a set of lines of a plant;
   (b) breeding the lines using traditional plant breeding methods to produce a set of candidate lines of the plant;
   (c) selecting two or more candidate lines together comprising three or more haplotypes;
   (d) generating a first parent MiMe plant and a second parent MiMe plant from the two or more candidate lines that together comprise the three or more haplotypes;
   (e) providing clonal gametes from each of the first and second parent MiMe plants;
   (f) crossing the clonal gametes to produce a hybrid polyploid seed comprising the three or more haplotypes;
   (g) growing the hybrid polyploid seed to produce a hybrid polyploid plant comprising three or more haplotypes; and
   (h) evaluating one or more characteristics of the hybrid polyploid plant; the method optionally further comprising
   (i) repeating steps (b)-(h) or steps (c)-(h) using the one or more characteristics of the hybrid polyploid plant evaluated in step (h) to guide the breeding of lines of step (b), the selecting of candidate lines of step (c), or both.
18. The method of embodiment 17, wherein:
   (A)
      (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only Mime alleles at one or more MiMe loci of a third MiMe component; and
      (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein either (i) at least one of the MiMe loci having only MiMe alleles of the third MiMe component of the first parent MiMe plant is the same as at least one of the Mime loci having only Mime alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
   (B)
      (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
      (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
   (C)
      (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
      (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
19. A method of breeding a polyploid hybrid plant line, the method comprising:
   (a) obtaining a set of lines of a plant;
   (b) breeding the lines using traditional plant breeding methods to produce a set of candidate lines of the plant;
   (c) selecting two or more candidate lines together comprising three or more haplotypes;
   (d) generating a first parent MiMe plant from one of the two or more candidate lines;
   (e) providing clonal gametes from the first parent MiMe plant;
   (f) providing haploid gametes from a homozygous parent non-MiMe plant of one of the two or more candidate lines;
   (g) crossing the clonal gametes with the haploid gametes to produce a hybrid polyploid seed;
   (h) growing the hybrid polyploid seed to produce a hybrid polyploid plant; and
   (i) evaluating one or more characteristics of the hybrid polyploid plant, wherein the first parent MiMe plant and the homozygous parent non-MiMe plant together comprise three or more haplotypes, wherein the crossing of step (g) results in the hybrid polyploid seed comprising three or more haplotypes, and wherein the growing of step (h) results in the hybrid polyploid plant comprising three or more haplotypes; the method optionally further comprising
   (j) repeating steps (b)-(i) or steps (c)-(i) using the one or more characteristics of the hybrid polyploid plant evaluated in step (i) to guide the breeding of lines of step (b), the selecting of candidate lines of step (c), or both.
20. The method of any one of embodiments 14-19, wherein the first parent MiMe plant, the second parent MiMe plant, the parent non-MiMe plant, or any combination thereof are diploid, triploid, or tetraploid and the hybrid polyploid plant is tetraploid, pentaploid, hexaploid, heptaploid, or octoploid.
21. The method of any one of embodiments 14-20, wherein generating the first parent MiMe plant, the second parent MiMe plant, or both comprises:
   (1) introducing a complete MiMe genotype directly into two candidate lines to produce the first parent MiMe plant, the second parent MiMe plant, or both;
   (2) introducing a partial MiMe genotype into two candidate lines to produce two grandparent non-MiMe plants each having a partial MiMe genotype and crossing the grandparent non-MiMe plants each having a partial MiMe genotype to produce the first parent MiMe plant, optionally wherein generating the first parent MiMe plant, the second parent MiMe plant, or both further comprises introducing a complete MiMe genotype directly into a third candidate line to produce the second parent MiMe plant; or
   (3) introducing a partial MiMe genotype into four candidate lines to produce four grandparent non-MiMe plants each having a partial MiMe genotype, and crossing pairs of said grandparent non-MiMe plants each having a partial MiMe genotype to produce the first and second parent MiMe plants, optionally wherein generating the first parent Mime plant, the second parent Mime plant, or both further comprises propagating the first parent MiMe plant, the second parent MiMe plant, the grandparent non-MiMe plants, or any combination thereof to scale production of homogenous seed.
22. The method of any one of embodiments 14-21, wherein the first parent MiMe plant, the second parent MiMe plant, the hybrid polyploid plant, or any combination thereof has a complete MiMe genotype comprising:
   (A)
   MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
   (B)
   MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and
   (4) a component of progression through the first division of meiosis, and each of the first MiMe component and the second MiMe component are different MiMe components.
23. The method of any one of embodiments 17-22, wherein each grandparent non-MiMe plant, the hybrid polyploid plant, or a combination thereof has a partial MiMe genotype comprising:
   (A)
      (a) one or more MiMe alleles conferring decreased expression of one or more MiMe loci of each of a first, second, and third MiMe component; and
      (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
   (B)
      (a) one or more MiMe alleles conferring decreased expression of one or more MiMe loci of each of the first and second MiMe component; and
      (b) one or more non-MiMe alleles at the one or more MiMe loci of each of the first and second Mime component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.
24. The method of embodiment 18, 22, or 23, wherein:
   (1) the one or more MiMe loci of the component of sister chromatid cohesion during the first division of meiosis comprise REC8, SWITCH1/DYAD, or a combination thereof;
   (2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
   (3) the one or more MiMe loci of the component of progression through the second division of meiosis comprise OSD1, CYCA1, TDM1, PC1, PC2, FC, or any combination thereof; and/or
   (4) the one or more MiMe loci of the component of progression through the first division of meiosis comprise PS1, JASON, or a combination thereof.
25. The method of any one of embodiments 17-24, wherein the hybrid polyploid plant comprises:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the Mime alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the hybrid polyploid plant is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the hybrid polyploid plant is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (15) a complete MiMe genotype comprising (i) a ps allele, wherein the hybrid polyploid plant is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the hybrid polyploid plant is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (20) a partially complemented MiMe genotype comprising (i) a ps allele, wherein the hybrid polyploid plant is heterozygous for the ps allele, (ii) an os allele, wherein the hybrid polyploid plant is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the hybrid polyploid plant is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
26. The method of any one of embodiments 17-25, wherein one or more of the candidate lines of step (c) are inbred lines, hybrid lines, or a combination thereof.
27. The method of any one of embodiments 21-26, wherein the complete MiMe genotype, the partial MiMe genotype, or both are introduced by gene editing, transgenesis, or a combination thereof.
28. The method of embodiment 22-27, wherein the decreased expression of one or more of the MiMe loci is each independently achieved by gene disruption, gene knockout, gene knockdown, gene silencing, RNA interference, or induction of methylation.
29. The method of embodiment 22-28, wherein the decreased expression of one or more of the MiMe loci is each independently achieved by introducing into each candidate line or a progenitor thereof an insertion, a deletion, one or more nucleotide changes, or an inversion that results in decreased expression of the MiMe locus, optionally including a step of selection for decreased expression of the MiMe locus.
30. The method of embodiment 29, wherein the insertion, the deletion, the one or more nucleotide changes, or the inversion eliminates expression of the MiMe locus, optionally wherein the expression of the MiMe locus is eliminated by a premature stop codon present in the 70%, the first 60%, the first 50%, the first 40%, the first 30%, the first 20%, or the first 10% of the nucleotides of the coding sequence of the Mime locus following the start codon in the 3′ direction.
31. A method of producing a population of polyploid seed comprising:
   (a) providing clonal gametes from a pair of parent MiMe plants together comprising three or more haplotypes that were selected using the method of breeding of any one of embodiments 17, 18, and 20-30 based upon the polyploid plant comprising the three or more haplotypes having one or more desired characteristics; and
   (b) crossing the clonal gametes to produce the population of polyploid seed;
   wherein the population of polyploid seed comprises a subpopulation of genetically uniform polyploid seed in an amount of at least 50% of the total number of seeds, the subpopulation of genetically uniform polyploid seed comprising the three or more haplotypes.
32. A method of producing a population of polyploid seed comprising:
   (a) selecting three or more haplotypes using the method of breeding of any one of embodiments 19-30 based upon the polyploid plant comprising said three or more haplotypes having one or more desired characteristics;
   (b) providing clonal gametes from a parent MiMe plant;
   (c) providing haploid gametes from a homozygous parent non-MiMe plant;
   (d) crossing the clonal gametes with the haploid gametes to produce the population of polyploid seed;
   wherein the parent MiMe plant and the homozygous parent non-MiMe plant together comprise the three or more haplotypes selected in step (a), wherein the crossing of step (d) results in a population of polyploid seed comprising a subpopulation of genetically uniform polyploid seed in an amount of at least 50% of the total number of seeds, the subpopulation of genetically uniform polyploid seed comprising the three or more haplotypes.
33. The method of embodiment 31 or 32, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.
34. The method of any one of embodiments 31-33, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.
35. The method of any one of embodiments 31-34, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.
36. A genetically modified plant, plant part, or plant cell comprising:
   (A) three or more haplotypes; and
   (B)
      (i) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
      ii) a partial MiMe genotype comprising:
         (a) one or more MiMe alleles conferring decreased expression of one or more MiMe loci of each of a first, second, and third MiMe component; and
         (b) one or more non-MiMe alleles at the one or more MiMe loci of each of the first, second, and third MiMe component,
         wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
      iii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components;
      iv) a partial MiMe genotype comprising:
         (a) one or more MiMe alleles conferring decreased expression of one or more Mime loci of each of the first and second MiMe component; and
         (b) one or more non-MiMe alleles at the one or more MiMe loci of each of the first and second MiMe component,
         wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components;
      v) a partially complemented MiMe genotype comprising:
         (a) only MiMe alleles at one or more MiMe loci of a first Mime component;
         (b) one or more Mime alleles and one or more non-MiMe alleles at a first Mime locus of a second MiMe component, and one or more MiMe alleles and one or more non-MiMe alleles at a second MiMe locus of the second MiMe component; and
         (c) either (i) only Mime alleles at one or more MiMe loci of a third Mime component, or (ii) one or more Mime alleles and one or more non-MiMe alleles at a first Mime locus of the third MiMe component, and one or more MiMe alleles and one or more non-MiMe alleles at a second MiMe locus of the third MiMe component,
         wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
      vi) a partially complemented MiMe genotype comprising:
         (a) only MiMe alleles at one or more MiMe loci of a first Mime component; and
         (b) one or more Mime alleles and one or more non-MiMe alleles at a first Mime locus of a second MiMe component, and one or more MiMe alleles and one or more non-MiMe alleles at a second MiMe locus of the second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; or
      vii) a partially complemented MiMe genotype comprising:
         (a) only MiMe alleles at one or more MiMe loci of a first Mime component, wherein the first MiMe component is a component of DNA double strand breakage during meiotic recombination;
         (b) one or more Mime alleles and one or more non-MiMe alleles at one or more Mime loci of a second MiMe component;
         (c) one or more MiMe alleles and one or more non-MiMe alleles at one or more MiMe loci of a third MiMe component; and
         (d) one or more Mime alleles and one or more non-MiMe alleles at one or more Mime loci of a fourth MiMe component,
         wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
37. The genetically modified plant, plant part, or plant cell of embodiment 36, wherein:
   (1) the one or more MiMe loci of the component of sister chromatid cohesion during the first division of meiosis comprise REC8, SWITCH1/DYAD, or a combination thereof;
   (2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
   (3) the one or more MiMe loci of the component of progression through the second division of meiosis comprise OSD1, CYCA1, TDM1, PC1, PC2, FC, or any combination thereof; and/or
   (4) the one or more MiMe loci of the component of progression through the first division of meiosis comprise PS1, JASON, or a combination thereof.
38. The genetically modified plant, plant part, or plant cell of embodiment 36 or 37, comprising:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the genetically modified plant, plant part, or plant cell is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the genetically modified plant, plant part, or plant cell is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO111-1;
   (15) a complete MiMe genotype comprising (i) a ps allele, wherein the genetically modified plant, plant part, or plant cell is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the genetically modified plant, plant part, or plant cell is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (20) a partially complemented MiMe genotype comprising (i) a ps allele, wherein genetically modified plant, plant part, or plant cell is heterozygous for the ps allele, (ii) an os allele, wherein genetically modified plant, plant part, or plant cell is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the genetically modified plant, plant part, or plant cell is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the Mime alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
39. The genetically modified plant, plant part, or plant cell of any one of embodiments 36-38, comprising one or more polynucleotide sequences selected from the group consisting of SEQ ID NOs: 448-466, 468-471, 474-491, and 493-496.
40. The genetically modified plant, plant part, or plant cell of any one of embodiments 36-39, wherein:
   (i) the genetically modified plant part is a non-regenerable plant part; or
   (ii) the genetically modified plant cell is a non-regenerable plant cell.
41. The genetically modified plant, plant part, or plant cell of any one of embodiments 36-40, wherein the plant part is a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, or a portion thereof.
42. A processed plant product derived from the genetically modified plant, plant part, or plant cell of any one of embodiments 36-41 wherein the processed plant product comprises a detectable amount of the one or more MiMe alleles of the genetically modified plant, plant part, or plant cell.
43. The processed plant product of embodiment 42, wherein the processed plant product:
   (i) is selected from the group consisting of plant biomass, oil, meal, food starch, syrup, animal feed, flour, flakes, bran, lint, hulls, and processed seed; and/or
   (ii) is non-regenerable.
44. Germplasm of the population of polyploid seeds of any one of embodiments 1-13.
45. A population of polyploid seed comprising a subpopulation of genetically uniform polyploid seed in an amount of at least 50% of the total number of seeds, the genetically uniform polyploid seed comprising three or more haplotypes of the same or related species of plant, wherein the population was obtained from a single plant or a set of F1 hybrids, wherein the subpopulation of genetically uniform polyploid seed comprise (A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or (B) (2) a germplasm genetic modification means for preventing DNA double strand breakage during meiotic recombination, and (4) a germplasm genetic modification means for preventing progression through the first division of meiosis;
   wherein each of the germplasm genetic modification means are (i) a complete set that achieves the inhibition, (ii) a partial set that does not achieve inhibition, or (iii) partially complemented at one or more of the genetic modification means and achieve sterility.
46. The population of embodiment 45, wherein the subpopulation of genetically uniform polyploid seed is triploid, tetraploid, pentaploid, hexaploid, heptaploid, or octoploid.
47. The population of embodiment 45 or 46, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.
48. The population of any one of embodiments 45-47, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.
49. The population of embodiment 45-48, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.
50. The population of any one of embodiments 45-49, wherein the subpopulation of genetically uniform polyploid seed has a complete MiMe genotype comprising the complete set of germplasm genetic modification means as follows:
   (A)
   (1) the germplasm genetic modification means for inhibiting sister chromatid cohesion during the first division of meiosis comprise only MiMe alleles at one or more MiMe loci of a component of sister chromatid cohesion during the first division of meiosis, (2) the germplasm genetic modification means for inhibiting DNA double strand breakage during meiotic recombination comprise only MiMe alleles at one or more MiMe loci of a component of DNA double strand breakage during meiotic recombination, and (3) the germplasm genetic modification means for inhibiting progression through the second division of meiosis comprise only MiMe alleles at one or more Mime loci of progression through the second division of meiosis; or
   (B)
   (2) the germplasm genetic modification means for inhibiting DNA double strand breakage during meiotic recombination comprise only MiMe alleles at one or more MiMe loci of a component of DNA double strand breakage during meiotic recombination, and (4) the germplasm genetic modification means for inhibiting progression through the first division of meiosis comprise only MiMe alleles at one or more MiMe loci of a component of progression through the first division of meiosis.
51. The population of any one of embodiments 45-49, wherein the subpopulation of genetically uniform polyploid seed has a partial MiMe genotype comprising the partial set of germplasm genetic modification means as follows:
   (A)
   (1) the germplasm genetic modification means for inhibiting sister chromatid cohesion during the first division of meiosis comprise at least one MiMe allele and at least one non-MiMe allele at one or more MiMe loci for a component of sister chromatid cohesion during the first division of meiosis comprise, (2) the germplasm genetic modification means for inhibiting DNA double strand breakage during meiotic recombination comprise at least one MiMe allele and at least one non-MiMe allele at one or more MiMe loci for a component of DNA double strand breakage during meiotic recombination, and (3) the germplasm genetic modification means for inhibiting progression through the second division of meiosis comprise at least one MiMe allele and at least one non-MiMe allele at one or more MiMe loci for a component of progression through the second division of meiosis; or
   (B)
   (2) the germplasm genetic modification means for inhibiting DNA double strand breakage during meiotic recombination comprise at least one MiMe allele and at least one non-MiMe allele at one or more MiMe loci for a component of DNA double strand breakage during meiotic recombination, and (4) the germplasm genetic modification means for inhibiting progression through the first division of meiosis comprise at least one MiMe allele and at least one non-MiMe allele at one or more MiMe loci for a component of progression through the first division of meiosis.
52. The population of any one of embodiments 45-49, wherein the subpopulation of genetically uniform polyploid seed has a partially-complemented MiMe genotype comprising the partially complemented set of germplasm genetic modification means as follows:
   (A)
      (1) the germplasm genetic modification means for inhibiting sister chromatid cohesion during the first division of meiosis comprises at least one MiMe allele at at least a first MiMe locus of a component of sister chromatid cohesion during the first division of meiosis, (2) the germplasm genetic modification means for inhibiting DNA double strand breakage during meiotic recombination comprises at least one MiMe allele at at least a first MiMe locus of a component of DNA double strand breakage during meiotic recombination, and (3) the germplasm genetic modification means for inhibiting progression through the second division of meiosis comprises at least one MiMe allele at at least a first MiMe locus of a component of progression through the second division of meiosis, wherein at least one of (1), (2), and (3) comprise one or more MiMe alleles and one or more non-MiMe alleles at the first MiMe locus of the component, and one or more MiMe alleles and one or more non-MiMe alleles at a second MiMe locus of the component, and wherein one or both of the others of (1),
      (2), and (3) comprise only MiMe alleles at at least the first Mime locus of the component; or
   (B)
      (2) the germplasm genetic modification means for inhibiting DNA double strand breakage during meiotic recombination comprises at least one MiMe allele at at least a first MiMe locus of a component of DNA double strand breakage during meiotic recombination, and (4) the germplasm genetic modification means for preventing progression through the first division of meiosis comprises at least one MiMe allele at at least a first MiMe locus of a component of progression through the first division of meiosis,
      wherein one of (2) and (4) comprises one or more MiMe alleles and one or more non-MiMe alleles at the first MiMe locus of the component, and one or more MiMe alleles and one or more non-MiMe alleles at a second MiMe locus of the component, and the other of (2) and (4) comprises only MiMe alleles at at least the first MiMe locus of the component; or
   (C)
      (2) the germplasm genetic modification means for inhibiting DNA double strand breakage during meiotic recombination comprise at least one MiMe allele at at least a first MiMe locus of a component of DNA double strand breakage during meiotic recombination;
      (1) the germplasm genetic modification means for inhibiting sister chromatid cohesion during the first division of meiosis comprises at least one MiMe allele and at least one non-MiMe allele at one or more MiMe loci for a component of sister chromatid cohesion during the first division of meiosis;
      (3) the germplasm genetic modification means for inhibiting progression through the second division of meiosis comprises at least one MiMe allele and at least one non-MiMe allele at one or more Mime loci for a component of progression through the second division of meiosis; and
      (4) the germplasm genetic modification means for inhibiting progression through the first division of meiosis comprise at least one MiMe allele and at least one non-MiMe allele at one or more Mime loci for a component of progression through the first division of meiosis.
53. The population of any one of embodiments 50-52, wherein:
   (1) the one or more MiMe loci of the component of sister chromatid cohesion during the first division of meiosis comprise REC8, SWITCH1/DYAD, or a combination thereof;
   (2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
   (3) the one or more MiMe loci of the component of progression through the second division of meiosis comprise OSD1, CYCA1, TDM1, PC1, PC2, FC, or any combination thereof; and/or
   (4) the one or more MiMe loci of the component of progression through the first division of meiosis comprise PS1, JASON, or a combination thereof.
54. The population of any one of embodiments 45-53, wherein the subpopulation of genetically uniform polyploid seed comprises:
   (1) a complete set of germplasm genetic modification means comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial set of germplasm genetic modification means comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete set of germplasm genetic modification means comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only Mime alleles at one or more SPO11-1 loci, wherein each of the MiMe alleles at the one or more SPO11-1 loci comprise one or more genetic modifications resulting in decreased expression of SPO11-1;
   (4) a partial set of germplasm genetic modification means comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete set of germplasm genetic modification means comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial set of germplasm genetic modification means comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete set of germplasm genetic modification means comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial set of germplasm genetic modification means comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete set of germplasm genetic modification means comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial set of germplasm genetic modification means comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete set of germplasm genetic modification means comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial set of germplasm genetic modification means comprising (i) at least one MiMe allele and at least one non-MiMe allele at one or more TDM1 loci, wherein each of the Mime alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete set of germplasm genetic modification means comprising (i) an os allele, wherein the subpopulation of genetically uniforms seed is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial set of germplasm genetic modification means comprising (i) an os allele, wherein the subpopulation of genetically uniforms seed is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (15) a complete set of germplasm genetic modification means comprising (i) aps allele, wherein the subpopulation of genetically uniforms seed is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial set of germplasm genetic modification means comprising (i) a ps allele, wherein the subpopulation of genetically uniforms seed is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented set of germplasm genetic modification means comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the Mime alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented set of germplasm genetic modification means comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (19) a partially complemented set of germplasm genetic modification means comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the Mime alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (20) a partially complemented set of germplasm genetic modification means comprising (i) a ps allele, wherein the subpopulation of genetically uniforms seed is heterozygous for the ps allele, (ii) an os allele, wherein the subpopulation of genetically uniforms seed is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented set of germplasm genetic modification means comprising (i) an os allele, wherein the subpopulation of genetically uniforms seed is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of Spo11-1.
55. The population of any one of embodiments 45-54, wherein the subpopulation of genetically uniform polyploid seed comprises one or more polynucleotide sequences selected from the group consisting of SEQ ID NOs: 448-49.
56. The population of any one of embodiments 45-55, wherein germination of a seed of the subpopulation of genetically uniform polyploid seed results in a sterile plant that produces inviable gametes, seedless fruit, or a combination thereof.
57. The population of any one of embodiments 45-56, wherein the population of polyploid seed is from a plant selected from the group consisting of potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussel sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine, mandarin, clementine, coffee, cola, hazelnut, saffron, melon, cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycrass, green bean, bean, ground cherry, pine, pistachio nut, pea, turf grass, poplar, apricot, plum, prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.
58. A method of producing the population of polyploid seed of any one of embodiments 45-57, the method comprising:
   (0) generating a first parent MiMe plant and a second parent MiMe plant by introducing genetic modifications into germplasms of one or more candidate lines of a plant to generate the first parent MiMe plant, the second parent MiMe plant, and/or progenitors thereof, wherein the first parent MiMe plant and the second parent MiMe plant together comprise three or more haplotypes, and wherein each of the first parent MiMe plant, the second parent MiMe plant, and/or the progenitors thereof comprises:
      (A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or
      (B) (2) a germplasm genetic modification means for preventing DNA double strand breakage during meiotic recombination, and (4) a germplasm genetic modification means for preventing progression through the first division of meiosis;
      wherein each of the germplasm genetic modifications are (i) a complete set that achieves the inhibition, or (ii) a partial set that does not achieve inhibition;
   wherein if progenitors are generated, the progenitors are further crossed to generate the first parent MiMe plant, the second parent MiMe plant, or both, and wherein the generating the first parent MiMe plant, the second parent MiMe plant, and/or the progenitors thereof comprises selecting for plants comprising the germplasm genetic modification means after introducing the germplasm genetic modification means;
   (a) providing clonal gametes from the first parent MiMe plant and the second parent MiMe plant that together comprise the three or more haplotypes; and
   (b) crossing the clonal gametes to produce the population of polyploid seed;
   wherein the population of polyploid seed comprises a subpopulation of genetically uniform polyploid seed in an amount of at least 50% of the total number of seeds, the subpopulation of genetically uniform polyploid seed comprising the three or more haplotypes.
59. The method of embodiment 58, wherein the subpopulation of genetically uniform polyploid seed has a partially-complemented MiMe genotype, wherein:
   (A)
   (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
   (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
   wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
   (B)
   (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
   (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
   wherein at least one of the MiMe loci having only MiMe alleles of the first MiMe component of the first parent MiMe plant is the same as at least one of the MiMe loci having only MiMe alleles of the first MiMe component of the second parent MiMe plant;
   wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
   (C)
   (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
   (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
   wherein at least one of the Mime loci having only Mime alleles of the first MiMe component of the first parent Mime plant is the same as at least one of the Mime loci having only MiMe alleles of the first MiMe component of the second parent MiMe plant;
   wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
60. A method of producing the population of polyploid seed of any one of embodiments 45-57, the method comprising:
   (0) generating a first parent MiMe plant by introducing genetic modifications into germplasms of one or more candidate lines of a plant to generate the first parent Mime plant or progenitors thereof, and wherein each of the first parent MiMe plant or the progenitors thereof comprises:
      (A) (1) a germplasm genetic modification means for inhibiting sister chromatid cohesion during the first division of meiosis, (2) a germplasm genetic modification means for inhibiting DNA double strand breakage during meiotic recombination, and (3) a germplasm genetic modification means for inhibiting progression through the second division of meiosis; or
      (B) (2) a germplasm genetic modification means for preventing DNA double strand breakage during meiotic recombination, and (4) a germplasm genetic modification means for preventing progression through the first division of meiosis;
      wherein each of the germplasm genetic modifications are (i) a complete set that achieves the inhibition, (ii) a partial set that does not achieve inhibition, or (iii) partially complemented at one or more of the genetic modification means and achieve sterility;
   wherein if progenitors are generated, the progenitors are further crossed to generate the first parent Mime plant, and wherein the generating the first parent MiMe plant and/or the progenitors thereof comprises selecting for plants comprising the germplasm genetic modification means after introducing the germplasm genetic modification means;
   (a) providing clonal gametes from the first parent MiMe plant;
   (b) providing haploid gametes from a homozygous parent non-MiMe plant; and
   (c) crossing the clonal gametes with the haploid gametes to produce the population of polyploid seed, wherein the clonal gametes and the haploid gametes together comprise three or more haplotypes, optionally wherein step (a) comprises generating the first parent MiMe plant, the second parent MiMe plant, or both.
61. A method of breeding a polyploid hybrid plant line, the method comprising:
   (a) obtaining a set of lines of a plant;
   (b) breeding the lines using traditional plant breeding methods to produce a set of candidate lines of the plant;
   (c) selecting two or more candidate lines together comprising three or more haplotypes;
   (d) generating a first parent MiMe plant and a second parent MiMe plant from the two or more candidate lines that together comprise the three or more haplotypes;
   (e) providing clonal gametes from each of the first and second parent MiMe plants;
   (f) crossing the clonal gametes to produce a hybrid polyploid seed comprising the three or more haplotypes;
   (g) growing the hybrid polyploid seed to produce a hybrid polyploid plant comprising three or more haplotypes; and
   (h) evaluating one or more characteristics of the hybrid polyploid plant; the method optionally further comprising
   (i) repeating steps (b)-(h) or steps (c)-(h) using the one or more characteristics of the hybrid polyploid plant evaluated in step (h) to guide the breeding of lines of step (b), the selecting of candidate lines of step (c), or both.
62. The method of embodiment 61, wherein:
   (A)
      (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
      (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
   (B)
      (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
      (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
   (C)
      (a) the first parent MiMe plant has only MiMe alleles at one or more MiMe loci of a first MiMe component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
      (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
63. A method of breeding a polyploid hybrid plant line, the method comprising:
   (a) obtaining a set of lines of a plant;
   (b) breeding the lines using traditional plant breeding methods to produce a set of candidate lines of the plant;
   (c) selecting two or more candidate lines together comprising three or more haplotypes;
   (d) generating a first parent MiMe plant from one of the two or more candidate lines;
   (e) providing clonal gametes from the first parent MiMe plant;
   (f) providing haploid gametes from a homozygous parent non-MiMe plant of one of the two or more candidate lines;
   (g) crossing the clonal gametes with the haploid gametes to produce a hybrid polyploid seed;
   (h) growing the hybrid polyploid seed to produce a hybrid polyploid plant; and
   (i) evaluating one or more characteristics of the hybrid polyploid plant, wherein the first parent MiMe plant and the homozygous parent non-MiMe plant together comprise three or more haplotypes, wherein the crossing of step (g) results in the hybrid polyploid seed comprising three or more haplotypes, and wherein the growing of step (h) results in the hybrid polyploid plant comprising three or more haplotypes; the method optionally further comprising
   (j) repeating steps (b)-(i) or steps (c)-(i) using the one or more characteristics of the hybrid polyploid plant evaluated in step (i) to guide the breeding of lines of step (b), the selecting of candidate lines of step (c), or both.
64. The method of any one of embodiments 58-63, wherein the first parent MiMe plant, the second parent MiMe plant, the parent non-MiMe plant, or any combination thereof are diploid, triploid, or tetraploid and the hybrid polyploid plant is tetraploid, pentaploid, hexaploid, heptaploid, or octoploid.
65. The method of any one of embodiments 58-64, wherein generating the first parent MiMe plant, the second parent MiMe plant, or both comprises:
   (1) introducing a complete MiMe genotype directly into two candidate lines to produce the first parent MiMe plant, the second parent MiMe plant, or both;
   (2) introducing a partial MiMe genotype into two candidate lines to produce two grandparent non-MiMe plants each having a partial MiMe genotype and crossing the grandparent non-MiMe plants each having a partial MiMe genotype to produce the first parent Mime plant, optionally wherein generating the first parent MiMe plant, the second parent MiMe plant, or both further comprises introducing a complete MiMe genotype directly into a third candidate line to produce the second parent MiMe plant; or
   (3) introducing a partial MiMe genotype into four candidate lines to produce four grandparent non-MiMe plants each having a partial MiMe genotype, and crossing pairs of said grandparent non-MiMe plants each having a partial MiMe genotype to produce the first and second parent MiMe plants, optionally wherein generating the first parent Mime plant, the second parent Mime plant, or both further comprises propagating the first parent MiMe plant, the second parent MiMe plant, the grandparent non-MiMe plants, or any combination thereof to scale production of homogenous seed.
66. The method of any one of embodiments 58-65, wherein the first parent Mime plant, the second parent MiMe plant, the hybrid polyploid plant, or any combination thereof has a complete MiMe genotype comprising:
   (A)
   MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
   (B)
   MiMe alleles conferring decreased expression of one or more Mime loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components.
67. The method of any one of embodiments 61-66, wherein each grandparent non-MiMe plant, the hybrid polyploid plant, or a combination thereof has a partial MiMe genotype comprising:
   (A)
      (a) one or more MiMe alleles conferring decreased expression of one or more Mime loci of each of a first, second, and third MiMe component; and
      (b) one or more non-MiMe alleles at the one or more MiMe loci of each of the first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components; or
   (B)
      (a) one or more MiMe alleles conferring decreased expression of one or more Mime loci of each of the first and second MiMe component; and
      (b) one or more non-MiMe alleles at the one or more MiMe loci of each of the first and second MiMe component, wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components.
68. The method of embodiment 18, 66, or 67, wherein:
   (1) the one or more MiMe loci of the component of sister chromatid cohesion during the first division of meiosis comprise REC8, SWITCH1/DYAD, or a combination thereof;
   (2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
   (3) the one or more MiMe loci of the component of progression through the second division of meiosis comprise OSD1, CYCA1, TDM1, PC1, PC2, FC, or any combination thereof; and/or
   (4) the one or more MiMe loci of the component of progression through the first division of meiosis comprise PS1, JASON, or a combination thereof.
69. The method of any one of embodiments 61-68, wherein the hybrid polyploid plant comprises:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the Mime alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the hybrid polyploid plant is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the hybrid polyploid plant is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (15) a complete MiMe genotype comprising (i) a ps allele, wherein the hybrid polyploid plant is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the hybrid polyploid plant is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (20) a partially complemented MiMe genotype comprising (i) a ps allele, wherein the hybrid polyploid plant is heterozygous for the ps allele, (ii) an os allele, wherein the hybrid polyploid plant is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the hybrid polyploid plant is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
70. The method of any one of embodiments 61-69, wherein one or more of the candidate lines of step (c) are inbred lines, hybrid lines, or a combination thereof.
71. The method of any one of embodiments 65-70, wherein the complete MiMe genotype, the partial MiMe genotype, or both are introduced by gene editing, transgenesis, or a combination thereof.
72. The method of embodiment 66-71, wherein the decreased expression of one or more of the MiMe loci is each independently achieved by gene disruption, gene knockout, gene knockdown, gene silencing, RNA interference, or induction of methylation.
73. The method of embodiment 66-71, wherein the decreased expression of one or more of the MiMe loci is each independently achieved by introducing into each candidate line an insertion, a deletion, one or more nucleotide changes, or an inversion that results in decreased expression of the MiMe locus, optionally including a step of selection for decreased expression of the MiMe locus.
74. The method of embodiment 73, wherein the insertion, the deletion, the one or more nucleotide changes, or the inversion eliminates expression of the MiMe locus, optionally wherein the expression of the MiMe locus is eliminated by a premature stop codon present in the 70%, the first 60%, the first 50%, the first 40%, the first 30%, the first 20%, or the first 10% of the nucleotides of the coding sequence of the MiMe locus following the start codon in the 3′ direction.
75. A method of producing a population of polyploid seed comprising:
   (a) providing clonal gametes from a pair of parent MiMe plants together comprising three or more haplotypes that were selected using the method of breeding of any one of embodiments 61, 62, and 64-74 based upon the polyploid plant comprising the three or more haplotypes having one or more desired characteristics; and
   (b) crossing the clonal gametes to produce the population of polyploid seed;
   wherein the population of polyploid seed comprises a subpopulation of genetically uniform polyploid seed in an amount of at least 50% of the total number of seeds, the subpopulation of genetically uniform polyploid seed comprising the three or more haplotypes.
76. A method of producing a population of polyploid seed comprising:
   (a) selecting three or more haplotypes using the method of breeding of any one of embodiments 63-74 based upon the polyploid plant comprising said three or more haplotypes having one or more desired characteristics;
   (b) providing clonal gametes from a parent MiMe plant;
   (c) providing haploid gametes from a homozygous parent non-MiMe plant;
   (d) crossing the clonal gametes with the haploid gametes to produce the population of polyploid seed;
   wherein the parent MiMe plant and the homozygous parent non-MiMe plant together comprise the three or more haplotypes selected in step (a), wherein the crossing of step (d) results in a population of polyploid seed comprising a subpopulation of genetically uniform polyploid seed in an amount of at least 50% of the total number of seeds, the subpopulation of genetically uniform polyploid seed comprising the three or more haplotypes.
77. The method of embodiment 75 or 76, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.
78. The method of any one of embodiments 75-77, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.
79. The method of any one of embodiments 75-78, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.
80. A genetically modified plant, plant part, or plant cell comprising:
   (A) three or more haplotypes; and
   (B)
      (i) a complete MiMe genotype comprising MiMe alleles conferring decreased expression of one or more MiMe loci of each of a first, second, and third MiMe component, wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
      ii) a partial MiMe genotype comprising:
         (a) one or more MiMe alleles conferring decreased expression of one or more Mime loci of each of a first, second, and third MiMe component; and
         (b) one or more non-MiMe alleles at the one or more MiMe loci of each of the first, second, and third MiMe component,
         wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
      iii) a complete MiMe genotype comprising MiMe alleles conferring decreased expression of one or more MiMe loci of each of a first and second MiMe component, wherein the first MiMe component and the second MiMe component are selected from the group consisting of (2) a component of DNA double strand breakage during meiotic recombination and (4) a component of progression through the first division of meiosis, and each of the first MiMe component and the second MiMe component are different MiMe components;
      iv) a partial MiMe genotype comprising:
         (a) one or more MiMe alleles conferring decreased expression of one or more Mime loci of each of the first and second MiMe component; and
         (b) one or more non-MiMe alleles at the one or more MiMe loci of each of the first and second MiMe component,
         wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components;
      v) a partially complemented MiMe genotype comprising:
         (a) only MiMe alleles at one or more MiMe loci of a first Mime component;
         (b) one or more Mime alleles and one or more non-MiMe alleles at a first Mime locus of a second MiMe component, and one or more MiMe alleles and one or more non-MiMe alleles at a second MiMe locus of the second MiMe component; and
         (c) either (i) only Mime alleles at one or more MiMe loci of a third Mime component, or (ii) one or more Mime alleles and one or more non-MiMe alleles at a first Mime locus of the third MiMe component, and one or more MiMe alleles and one or more non-MiMe alleles at a second MiMe locus of the third MiMe component,
         wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components;
      vi) a partially complemented MiMe genotype comprising:
         (a) only MiMe alleles at one or more MiMe loci of a first Mime component; and
         (b) one or more Mime alleles and one or more non-MiMe alleles at a first Mime locus of a second MiMe component, and one or more MiMe alleles and one or more non-MiMe alleles at a second MiMe locus of the second MiMe component,
         wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; or
      vii) a partially complemented MiMe genotype comprising:
         (a) only MiMe alleles at one or more MiMe loci of a first Mime component, wherein the first MiMe component is a component of DNA double strand breakage during meiotic recombination;
         (b) one or more Mime alleles and one or more non-MiMe alleles at one or more Mime loci of a second MiMe component;
         (c) one or more MiMe alleles and one or more non-MiMe alleles at one or more MiMe loci of a third MiMe component; and
         (d) one or more Mime alleles and one or more non-MiMe alleles at one or more Mime loci of a fourth MiMe component,
         wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
81. The genetically modified plant, plant part, or plant cell of embodiment 80, wherein:
   (1) the one or more MiMe loci of the component of sister chromatid cohesion during the first division of meiosis comprise REC8, SWITCH1/DYAD, or a combination thereof;
   (2) the one or more MiMe loci of the component of DNA double strand breakage during meiotic recombination comprise PAIR1, SPO11-1, SPO11-2, PRD1, PRD2, DFO, MTOPVIB, DSY1, SY1, SY2, SY3, SY4, or any combination thereof;
   (3) the one or more MiMe loci of the component of progression through the second division of meiosis comprise OSD1, CYCA1, TDM1, PC1, PC2, FC, or any combination thereof; and/or
   (4) the one or more MiMe loci of the component of progression through the first division of meiosis comprise PS1, JASON, or a combination thereof.
82. The genetically modified plant, plant part, or plant cell of embodiment 80 or 81, comprising:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the genetically modified plant, plant part, or plant cell is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the genetically modified plant, plant part, or plant cell is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO111-1;
   (15) a complete MiMe genotype comprising (i) a ps allele, wherein the genetically modified plant, plant part, or plant cell is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the genetically modified plant, plant part, or plant cell is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (20) a partially complemented MiMe genotype comprising (i) a ps allele, wherein the genetically modified plant, plant part, or plant cell is heterozygous for the ps allele, (ii) an os allele, wherein the genetically modified plant, plant part, or plant cell is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the genetically modified plant, plant part, or plant cell is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
83. The genetically modified plant, plant part, or plant cell of any one of embodiments 80-82, comprising one or more polynucleotide sequences selected from the group consisting of SEQ ID NOs: 448-466, 468-471, 474-491, and 493-496.
84. The genetically modified plant, plant part, or plant cell of any one of embodiments 80-83, wherein:
   (i) the genetically modified plant part is a non-regenerable plant part; or
   (ii) the genetically modified plant cell is a non-regenerable plant cell.
85. The genetically modified plant, plant part, or plant cell of any one of embodiments 80-84, wherein the plant part is a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, or a portion thereof.
86. A processed plant product derived from the genetically modified plant, plant part, or plant cell of any one of embodiments 80-85 wherein the processed plant product comprises a detectable amount of the one or more MiMe alleles of the genetically modified plant, plant part, or plant cell.
87. The processed plant product of embodiment 86, wherein the processed plant product:
   (i) is selected from the group consisting of plant biomass, oil, meal, food starch, syrup, animal feed, flour, flakes, bran, lint, hulls, and processed seed; and/or
   (ii) is non-regenerable.
88. Germplasm of the population of polyploid seeds of any one of embodiments 45-59.
89. A population of polyploid seed comprising a subpopulation of genetically uniform polyploid seed in an amount of at least 50% of the total number of seeds, the genetically uniform polyploid seed comprising three or more haplotypes of the same or related species of plant, wherein the population was obtained from a single plant or a set of F1 hybrids, wherein the subpopulation of genetically uniform polyploid seed comprise
   (A) (1) one or more MiMe alleles at each of one or more MiMe loci of a component of sister chromatid cohesion during the first division of meiosis comprising REC8, SWITCH1/DYAD, or a combination thereof,
   (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
   (B) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
   wherein each of the of the Mime alleles comprises a germplasm genetic modification,
   wherein at least one seed, or a plant grown therefrom, of the population of polyploid seed has been selected for lack of expression of each of the one or more Mime loci, and
   wherein the subpopulation of genetically uniform polyploid seeds comprises (i) a complete MiMe genotype comprising only MiMe alleles at each of the one or more Mime loci, (ii) a partial Mime genotype comprising at least one MiMe allele and at least one non-MiMe allele at each of the one or more MiMe loci, or (iii) a partially complemented Mime genotype comprising only MiMe alleles at a first MiMe locus of the one or more MiMe loci and at least one Mime allele and at least one non-Mime allele at a second Mime locus of the one or more MiMe loci.
90. The population of embodiment 89, wherein the subpopulation of genetically uniform polyploid seed is triploid, tetraploid, pentaploid, hexaploid, heptaploid, or octoploid.
91. The population of embodiment 89 or 90, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.
92. The population of any one of embodiments 89-91, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.
93. The population of embodiment 89-92, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.
94. The population of any one of embodiments 89-93, wherein the subpopulation of genetically uniform polyploid seed has a partially-complemented MiMe genotype comprising:
   (A)
      (a) only MiMe alleles at one or more MiMe loci of a first Mime component;
      (b) one or more Mime alleles and one or more non-MiMe alleles at a first Mime locus of a second MiMe component, and one or more MiMe alleles and one or more non-MiMe alleles at a second MiMe locus of the second MiMe component; and
      (c) either (i) only Mime alleles at one or more Mime loci of a third Mime component, or (ii) one or more Mime alleles and one or more non-MiMe alleles at a first Mime locus of the third MiMe component, and one or more MiMe alleles and one or more non-MiMe alleles at a second MiMe locus of the third MiMe component,
      wherein the first MiMe component, the second MiMe component, and the third MiMe component are selected from the group consisting of (1) the component of sister chromatid cohesion during the first division of meiosis, (2) the component of DNA double strand breakage during meiotic recombination, and (3) the component of progression through the second division of meiosis, and each of the first MiMe component, the second MiMe component, and the third MiMe component are different MiMe components;
   (B)
      (a) only MiMe alleles at one or more MiMe loci of a first MiMe component; and
      (b) one or more Mime alleles and one or more non-MiMe alleles at a first Mime locus of a second MiMe component, and one or more MiMe alleles and one or more non-MiMe alleles at a second MiMe locus of the second MiMe component,
      wherein the first MiMe component and the second MiMe component are selected from the group consisting of (2) the component of DNA double strand breakage during meiotic recombination and (4) the component of progression through the first division of meiosis and each of the first MiMe component and the second MiMe component are different MiMe components; or
   (C)
      (a) only MiMe alleles at one or more MiMe loci of a first Mime component, wherein the first Mime component is the component of DNA double strand breakage during meiotic recombination;
      (b) one or more Mime alleles and one or more non-MiMe alleles at one or more Mime loci of a second MiMe component;
      (c) one or more MiMe alleles and one or more non-MiMe alleles at one or more MiMe loci of a third MiMe component; and
      (d) one or more Mime alleles and one or more non-MiMe alleles at one or more Mime loci of a fourth MiMe component,
      wherein the second MiMe component, the third MiMe component, and the fourth MiMe component are selected from the group consisting of (1) the component of sister chromatid cohesion during the first division of meiosis, (3) the component of progression through the second division of meiosis, and (4) the component of progression through the first division of meiosis, and each of the second MiMe component, the third MiMe component, and the fourth MiMe component are different MiMe components.
95. The population of any one of embodiments 89-94, wherein the subpopulation of genetically uniform polyploid seed comprises:
   (1) a complete Mime genotype comprising (i) only Mime alleles at one or more OSD1 loci, wherein each of the MiMe alleles at the one or more OSD1 loci comprise one or more genetic modifications resulting in decreased expression of OSD1, (ii) only Mime alleles at one or more REC8 loci, wherein each of the Mime alleles at the one or more REC8 loci comprise one or more genetic modifications resulting in decreased expression of REC8, and (iii) only Mime alleles at one or more PAIR1 loci, wherein each of the MiMe alleles at the one or more PAIR1 loci comprise one or more genetic modifications resulting in decreased expression of PAIR1;
   (2) a partial Mime genotype comprising (i) at least one Mime allele and at least one non-MiMe allele at one or more OSD1 loci, wherein each of the MiMe alleles at the one or more OSD1 loci comprise one or more genetic modifications resulting in decreased expression of OSD1, (ii) at least one Mime allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the Mime alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the subpopulation of genetically uniforms seed is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the subpopulation of genetically uniforms seed is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (15) a complete MiMe genotype comprising (i) a ps allele, wherein the subpopulation of genetically uniforms seed is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the subpopulation of genetically uniforms seed is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (20) a partially complemented MiMe genotype comprising (i) a ps allele, wherein the subpopulation of genetically uniforms seed is heterozygous for the ps allele, (ii) an os allele, wherein the subpopulation of genetically uniforms seed is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the subpopulation of genetically uniforms seed is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
96. The population of any one of embodiments 89-95, wherein the subpopulation of genetically uniform polyploid seed comprises one or more polynucleotide sequences selected from the group consisting of SEQ ID NOs: 448-466, 468-471, 474-491, and 493-496.
97. The population of any one of embodiments 89-96, wherein germination of a seed of the subpopulation of genetically uniform polyploid seed results in a sterile plant that produces inviable gametes, seedless fruit, or a combination thereof.
98. The population of any one of embodiments 89-97, wherein the population of polyploid seed is from a plant selected from the group consisting of potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, 353ashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussel sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine, mandarin, clementine, coffee, cola, hazelnut, saffron, melon, cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycrass, green bean, bean, ground cherry, pine, pistachio nut, pea, turf grass, poplar, apricot, plum, prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.
99. A method of producing the population of polyploid seed of any one of embodiments 89-98, the method comprising:
   (a) generating a first parent MiMe plant and a second parent MiMe plant by introducing genetic modifications into one or more candidate lines to produce MiMe alleles in germplasm of a first parent MiMe plant, a second parent MiMe plant, and/or progenitors thereof, wherein the first parent Mime plant and the second parent MiMe plant each comprise a complete MiMe genotype, and each of the progenitors comprises a partial MiMe genotype, wherein if progenitors are generated, the progenitors are further crossed to generate the first parent MiMe plant, the second parent Mime plant, or both;
   (b) providing clonal gametes from the first parent MiMe plant and the second parent MiMe plant that together comprise the three or more haplotypes; and
   (c) crossing the clonal gametes to produce the population of polyploid seed;
   wherein the population of polyploid seed comprises a subpopulation of genetically uniform polyploid seed in an amount of at least 50% of the total number of seeds, the subpopulation of genetically uniform polyploid seed comprising the three or more haplotypes.
100. The method of embodiment 99, wherein the subpopulation of genetically uniform polyploid seed has a partially-complemented MiMe genotype, wherein:
   (A)
      (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only Mime alleles at one or more MiMe loci of a third MiMe component; and
      (b) the second parent MiMe plant has only MiMe alleles at one or more Mime loci of the first Mime component, only non-MiMe alleles at the first MiMe locus of the second MiMe component, only MiMe alleles at the second MiMe locus of the second MiMe component, and only MiMe alleles at one or more MiMe loci of the third MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein either (i) at least one of the MiMe loci having only MiMe alleles of the third MiMe component of the first parent MiMe plant is the same as at least one of the Mime loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
   (B)
      (a) the first parent MiMe plant has only Mime alleles at one or more MiMe loci of a first MiMe component, only MiMe alleles at a first MiMe locus of a second MiMe component, and only non-MiMe alleles at a second MiMe locus of the second MiMe component; and
      (b) the second parent MiMe plant has only MiMe alleles at one or more Mime loci of the first MiMe component, only non-MiMe alleles at the first MiMe locus of the second MiMe component, and only Mime alleles at the second MiMe locus of the second MiMe component;
      wherein at least one of the Mime loci having only Mime alleles of the first MiMe component of the first parent Mime plant is the same as at least one of the Mime loci having only MiMe alleles of the first MiMe component of the second parent MiMe plant;
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
   (C)
      (a) the first parent MiMe plant has only MiMe alleles at one or more Mime loci of a first Mime component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
      (b) the second parent MiMe plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
      wherein at least one of the Mime loci having only Mime alleles of the first MiMe component of the first parent Mime plant is the same as at least one of the MiMe loci having only Mime alleles of the first MiMe component of the second parent MiMe plant;
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
101. A method of producing the population of polyploid seed of any one of embodiments 1-98, the method comprising:
   (a) generating a first parent MiMe plant by introducing genetic modifications into one or more candidate lines to produce MiMe alleles in germplasm of the first parent MiMe plant or progenitors thereof, wherein the first parent MiMe plant comprises a complete MiMe genotype, and each of the progenitors comprises a partial MiMe genotype, wherein if progenitors are generated, the progenitors are further crossed to generate the first parent Mime plant;
   (b) providing clonal gametes from the parent MiMe plant;
   (c) providing haploid gametes from a homozygous parent non-MiMe plant; and
   (d) crossing the clonal gametes with the haploid gametes to produce the population of polyploid seed, wherein the clonal gametes and the haploid gametes together comprise three or more haplotypes, optionally wherein step (a) comprises generating the first parent MiMe plant, the second parent Mime plant, or both.
102. A method of breeding a polyploid hybrid plant line, the method comprising:
   (a) obtaining a set of lines of a plant;
   (b) breeding the lines using traditional plant breeding methods to produce a set of candidate lines of the plant;
   (c) selecting two or more candidate lines together comprising three or more haplotypes;
   (d) generating a first parent MiMe plant and a second parent MiMe plant from the two or more candidate lines that together comprise the three or more haplotypes;
   (e) providing clonal gametes from each of the first and second parent Mime plants;
   (f) crossing the clonal gametes to produce a hybrid polyploid seed comprising the three or more haplotypes;
   (g) growing the hybrid polyploid seed to produce a hybrid polyploid plant comprising three or more haplotypes; and
   (h) evaluating one or more characteristics of the hybrid polyploid plant, wherein the first parent MiMe plant, the second parent MiMe plant, and the hybrid polyploid plant each independently comprise:
(A) (1) one or more Mime alleles at each of one or more Mime loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
(B) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
wherein each of the of the Mime alleles comprises a germplasm genetic modification,
wherein the method comprises selecting the first parent MiMe plant, the second parent MiMe plant, the hybrid polyploid plant, or any combination thereof for lack of expression of the one or more MiMe loci, and wherein the first parent MiMe plant and the second parent MiMe plant each independently comprise a complete MiMe genotype comprising only MiMe alleles at each of the one or more MiMe loci, and
wherein the hybrid polyploid plant comprises (i) a complete MiMe genotype comprising only MiMe alleles at each of the one or more MiMe loci, or (ii) a partially complemented MiMe genotype comprising only MiMe alleles at a first MiMe locus of the one or more MiMe loci and at least one MiMe allele and at least one non-MiMe allele at a second MiMe locus of the one or more MiMe loci;
   the method optionally further comprising
   (i) repeating steps (b)-(h) or steps (c)-(h) using the one or more characteristics of the hybrid polyploid plant evaluated in step (h) to guide the breeding of lines of step (b), the selecting of candidate lines of step (c), or both.
103. The method of embodiment 102, wherein:
   (A)
      (a) the first parent MiMe plant has only MiMe alleles at one or more MiMe loci of a first MiMe component, only MiMe alleles at a first MiMe locus of a second MiMe component, only non-MiMe alleles at a second MiMe locus of the second MiMe component, and only MiMe alleles at one or more MiMe loci of a third MiMe component; and
      (b) the second parent MiMe plant has only MiMe alleles at one or more MiMe loci of the first MiMe component, only non-MiMe alleles at the first MiMe locus of the second MiMe component, only MiMe alleles at the second MiMe locus of the second MiMe component, and only MiMe alleles at one or more MiMe loci of the third MiMe component;
      wherein at least one of the MiMe loci having only MiMe alleles of the first MiMe component of the first parent MiMe plant is the same as at least one of the MiMe loci having only MiMe alleles of the first MiMe component of the second parent MiMe plant;
      wherein either (i) at least one of the MiMe loci having only MiMe alleles of the third MiMe component of the first parent MiMe plant is the same as at least one of the MiMe loci having only MiMe alleles of the third MiMe component of the second parent MiMe plant, or (ii) the one or more MiMe loci having only MiMe alleles of the third MiMe component of the first parent MiMe plant are distinct from the one or more MiMe loci having only MiMe alleles of the third MiMe component of the second parent MiMe plant;
      wherein the first MiMe component, the second MiMe component, and the third MiMe component are selected from the group consisting of (1) a component of sister chromatid cohesion during the first division of meiosis, (2) a component of DNA double strand breakage during meiotic recombination, and (3) a component of progression through the second division of meiosis, and each of the first MiMe component, the second MiMe component, and the third MiMe component are different MiMe components.
   (B)
      (a) the first parent MiMe plant has only MiMe alleles at one or more MiMe loci of a first MiMe component, only MiMe alleles at a first MiMe locus of a second MiMe component, and only non-MiMe alleles at a second MiMe locus of the second MiMe component; and
      (b) the second parent MiMe plant has only MiMe alleles at one or more MiMe loci of the first MiMe component, only non-MiMe alleles at the first MiMe locus of the second MiMe component, and only MiMe alleles at the second MiMe locus of the second MiMe component;
      wherein at least one of the Mime loci having only MiMe alleles of the first Mime component of the first parent Mime plant is the same as at least one of the Mime loci having only MiMe alleles of the first MiMe component of the second parent MiMe plant;
      wherein the first MiMe component and the second MiMe component are selected from the group consisting of (2) a component of DNA double strand breakage during meiotic recombination and (4) a component of progression through the first division of meiosis and each of the first MiMe component and the second MiMe component are different MiMe components; and
   (C)
      (a) the first parent MiMe plant has only MiMe alleles at one or more Mime loci of a first Mime component, only MiMe alleles one or more MiMe loci of a second MiMe component, only MiMe alleles at one or more MiMe loci of a third MiMe component, and only non-MiMe alleles at one or more MiMe loci of a fourth MiMe component, wherein the first MiMe component is a component of DNA double strand breakage during meiotic recombination;
      (b) the second parent Mime plant has only MiMe alleles at the one or more MiMe loci of the first MiMe component, only non-MiMe alleles at the one or more MiMe loci of the second MiMe component, only non-MiMe alleles at the one or more MiMe loci of the third MiMe component, and only MiMe alleles at the one or more MiMe loci of the fourth MiMe component; and
      wherein at least one of the Mime loci having only Mime alleles of the first MiMe component of the first parent Mime plant is the same as at least one of the Mime loci having only MiMe alleles of the first MiMe component of the second parent MiMe plant;
      wherein the second MiMe component, the third MiMe component, and the fourth MiMe component are selected from the group consisting of (1) a component of sister chromatid cohesion during the first division of meiosis, (3) a component of progression through the second division of meiosis, and (4) a component of progression through the first division of meiosis, and each of the second MiMe component, the third MiMe component, and the fourth MiMe component are different MiMe components.
104. A method of breeding a polyploid hybrid plant line, the method comprising:
   (a) obtaining a set of lines of a plant;
   (b) breeding the lines using traditional plant breeding methods to produce a set of candidate lines of the plant;
   (c) selecting two or more candidate lines together comprising three or more haplotypes;
   (d) generating a first parent MiMe plant from one of the two or more candidate lines;
   (e) providing clonal gametes from the first parent Mime plant;
   (f) providing haploid gametes from a homozygous parent non-MiMe plant of one of the two or more candidate lines;
   (g) crossing the clonal gametes with the haploid gametes to produce a hybrid polyploid seed;
   (h) growing the hybrid polyploid seed to produce a hybrid polyploid plant; and
   (i) evaluating one or more characteristics of the hybrid polyploid plant, wherein the first parent MiMe plant and the homozygous parent non-MiMe plant together comprise three or more haplotypes, wherein the crossing of step (g) results in the hybrid polyploid seed comprising three or more haplotypes, and wherein the growing of step (h) results in the hybrid polyploid plant comprising three or more haplotypes;
wherein the first parent MiMe plant and the hybrid polyploid plant each independently comprise:
(A) (1) one or more MiMe alleles at each of one or more MiMe loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
(B) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
wherein each of the of the MiMe alleles comprises a germplasm genetic modification,
wherein the method comprises screening the first parent MiMe plant, the hybrid polyploid plant, or any combination thereof for lack of expression of the one or more MiMe loci, and
wherein the first parent MiMe comprises a complete MiMe genotype comprising only MiMe alleles at each of the one or more MiMe loci, the parent non-MiMe plant comprises only non-MiMe alleles at each of the one or more MiMe loci, and the hybrid polyploid plant comprises a partial MiMe genotype comprising at least one MiMe allele and at least one non-MiMe allele at each of the one or more MiMe loci, the method optionally further comprising
   j) repeating steps (b)-(i) or steps (c)-(i) using the one or more characteristics of the hybrid polyploid plant evaluated in step (i) to guide the breeding of lines of step (b), the selecting of candidate lines of step (c), or both.
105. The method of any one of embodiments 101-104, wherein the first parent MiMe plant, the second parent MiMe plant, the parent non-MiMe plant, or any combination thereof are diploid, triploid, or tetraploid and the hybrid polyploid plant is tetraploid, pentaploid, hexaploid, heptaploid, or octoploid.
106. The method of any one of embodiments 101-105, wherein generating the first parent MiMe plant, the second parent MiMe plant, or both comprises:
   (1) introducing a complete MiMe genotype directly into two candidate lines to produce the first parent MiMe plant, the second parent MiMe plant, or both;
   (2) introducing a partial MiMe genotype into two candidate lines to produce two grandparent non-MiMe plants each having a partial MiMe genotype and crossing the grandparent non-MiMe plants each having a partial MiMe genotype to produce the first parent MiMe plant, optionally wherein generating the first parent MiMe plant, the second parent MiMe plant, or both further comprises introducing a complete MiMe genotype directly into a third candidate line to produce the second parent MiMe plant; or
   (3) introducing a partial MiMe genotype into four candidate lines to produce four grandparent non-MiMe plants each having a partial MiMe genotype, and crossing pairs of said grandparent non-MiMe plants each having a partial MiMe genotype to produce the first and second parent MiMe plants, optionally wherein generating the first parent MiMe plant, the second parent MiMe plant, or both further comprises propagating the first parent MiMe plant, the second parent MiMe plant, the grandparent non-MiMe plants, or any combination thereof to scale production of homogenous seed.
107. The method of any one of embodiments 102-106, wherein the hybrid polyploid plant comprises:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the Mime alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the polyploid hybrid plant is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the Mime alleles at the one or more SPO11-1 loci comprise one or more genetic modifications resulting in decreased expression of SPO11-1;
   (14) a partial MiMe genotype comprising (i) an os allele, wherein the polyploid hybrid plant is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (15) a complete MiMe genotype comprising (i) a ps allele, wherein the hybrid polyploid plant is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the hybrid polyploid plant is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (20) a partially complemented MiMe genotype comprising (i) a ps allele, wherein the hybrid polyploid plant is heterozygous for the ps allele, (ii) an os allele, wherein the polyploid hybrid plant is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the polyploid hybrid plant is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
108. The method of any one of embodiments 102-107, wherein one or more of the candidate lines of step (c) are inbred lines, hybrid lines, or a combination thereof.
109. The method of any one of embodiments 102-108, wherein the complete MiMe genotype, the partial MiMe genotype, or both are introduced by gene editing, transgenesis, or a combination thereof.
110. The method of embodiment 102-109, wherein the decreased expression of one or more of the MiMe loci is each independently achieved by gene disruption, gene knockout, gene knockdown, gene silencing, RNA interference, or induction of methylation.
111. The method of embodiment 102-110, wherein the decreased expression of one or more of the MiMe loci is each independently achieved by introducing into each candidate line or a progenitor thereof an insertion, a deletion, one or more nucleotide changes, or an inversion that results in decreased expression of the MiMe locus, optionally including a step of selection for decreased expression of the MiMe locus.
112. The method of embodiment 111, wherein the insertion, the deletion, the one or more nucleotide changes, or the inversion eliminates expression of the MiMe locus, optionally wherein the expression of the MiMe locus is eliminated by a premature stop codon present in the 70%, the first 60%, the first 50%, the first 40%, the first 30%, the first 20%, or the first 10% of the nucleotides of the coding sequence of the Mime locus following the start codon in the 3′ direction.
113. A method of producing a population of polyploid seed comprising:
   (a) providing clonal gametes from a pair of parent MiMe plants together comprising three or more haplotypes that were selected using the method of breeding of any one of embodiments 102, 103, and 105-112 based upon the polyploid plant comprising the three or more haplotypes having one or more desired characteristics; and
   (b) crossing the clonal gametes to produce the population of polyploid seed;
   wherein the population of polyploid seed comprises a subpopulation of genetically uniform polyploid seed in an amount of at least 50% of the total number of seeds, the subpopulation of genetically uniform polyploid seed comprising the three or more haplotypes.
114. A method of producing a population of polyploid seed comprising:
   (a) selecting three or more haplotypes using the method of breeding of any one of embodiments 104-112 based upon the polyploid plant comprising said three or more haplotypes having one or more desired characteristics;
   (b) providing clonal gametes from a parent MiMe plant;
   (c) providing haploid gametes from a homozygous parent non-MiMe plant;
   (d) crossing the clonal gametes with the haploid gametes to produce the population of polyploid seed;
   wherein the parent MiMe plant and the homozygous parent non-MiMe plant together comprise the three or more haplotypes selected in step (a), wherein the crossing of step (d) results in a population of polyploid seed comprising a subpopulation of genetically uniform polyploid seed in an amount of at least 50% of the total number of seeds, the subpopulation of genetically uniform polyploid seed comprising the three or more haplotypes.
115. The method of embodiment 113 or 114, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.
116. The method of any one of embodiments 113-115, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.
117. The method of any one of embodiments 113-116, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.
118. A genetically modified plant, plant part, or plant cell comprising:
   (A) three or more haplotypes; and
   (B)
      (a) (1) one or more Mime alleles at each of one or more Mime loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
      (b) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
      wherein each of the of the MiMe alleles comprises a germplasm genetic modification comprising introduction of a premature in-frame stop codon present in the first 30% of the nucleotides of the coding sequence of the MiMe locus following the start codon in the 3′ direction, thereby generating a MiMe allele, and
      wherein the subpopulation of genetically uniform polyploid seeds comprises (i) a complete MiMe genotype comprising only MiMe alleles at each of the one or more Mime loci, (ii) a partial Mime genotype comprising at least one MiMe allele and at least one non-MiMe allele at each of the one or more MiMe loci, or (iii) a partially complemented Mime genotype comprising only MiMe alleles at a first MiMe locus of the one or more MiMe loci and at least one MiMe allele and at least one non-Mime allele at a second Mime locus of the one or more MiMe loci
      wherein the partially-complemented MiMe genotype comprises:
      (i)
         (a) only MiMe alleles at one or more MiMe loci of a first Mime component;
         (b) one or more Mime alleles and one or more non-MiMe alleles at a first Mime locus of a second MiMe component, and one or more MiMe alleles and one or more non-MiMe alleles at a second MiMe locus of the second MiMe component; and
         (c) either (i) only MiMe alleles at one or more MiMe loci of a third MiMe component, or (ii) one or more MiMe alleles and one or more non-MiMe alleles at a first MiMe locus of the third MiMe component, and one or more MiMe alleles and one or more non-MiMe alleles at a second MiMe locus of the third MiMe component,
         wherein the first MiMe component, the second MiMe component, and the third MiMe component are selected from the group consisting of (1) the component of sister chromatid cohesion during the first division of meiosis, (2) the component of DNA double strand breakage during meiotic recombination, and (3) the component of progression through the second division of meiosis, and each of the first MiMe component, the second MiMe component, and the third MiMe component are different MiMe components;
      (ii)
         (a) only MiMe alleles at one or more MiMe loci of a first MiMe component; and
         (b) one or more MiMe alleles and one or more non-MiMe alleles at a first Mime locus of a second MiMe component, and one or more MiMe alleles and one or more non-MiMe alleles at a second MiMe locus of the second MiMe component,
         wherein the first MiMe component and the second MiMe component are selected from the group consisting of (2) the component of DNA double strand breakage during meiotic recombination and (4) the component of progression through the first division of meiosis and each of the first MiMe component and the second MiMe component are different MiMe components; or
      (iii)
         (a) only MiMe alleles at one or more MiMe loci of a first MiMe component, wherein the first MiMe component is the component of DNA double strand breakage during meiotic recombination;
         (b) one or more MiMe alleles and one or more non-MiMe alleles at one or more MiMe loci of a second MiMe component;
         (c) one or more MiMe alleles and one or more non-MiMe alleles at one or more MiMe loci of a third MiMe component; and
         (d) one or more MiMe alleles and one or more non-MiMe alleles at one or more MiMe loci of a fourth MiMe component,
         wherein the second MiMe component, the third MiMe component, and the fourth MiMe component are selected from the group consisting of (1) the component of sister chromatid cohesion during the first division of meiosis, (3) the component of progression through the second division of meiosis, and (4) the component of progression through the first division of meiosis, and each of the second MiMe component, the third MiMe component, and the fourth MiMe component are different MiMe components.
119. The genetically modified plant, plant part, or plant cell of embodiment 118, comprising:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2;
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the Mime alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the genetically modified plant, plant part, or plant cell is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the genetically modified plant, plant part, or plant cell is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (15) a complete MiMe genotype comprising (i) a ps allele, wherein the genetically modified plant, plant part, or plant cell is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the genetically modified plant, plant part, or plant cell is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (20) a partially complemented MiMe genotype comprising (i) a ps allele, wherein the genetically modified plant, plant part, or plant cell is heterozygous for the ps allele, (ii) an os allele, wherein the genetically modified plant, plant part, or plant cell is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the Mime alleles at the SPO11-1 locus comprise one or more genetic modifications resulting in decreased expression of SPO11-1; or
   (21) a partially complemented MiMe genotype comprising (i) an os allele, wherein genetically modified plant, plant part, or plant cell is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
120. The genetically modified plant, plant part, or plant cell of embodiment 118 or 119, comprising one or more polynucleotide sequences selected from the group consisting of SEQ ID NOs: 448-466, 468-471, 474-491, and 493-496.
121. The genetically modified plant, plant part, or plant cell of any one of embodiments 118-120, wherein:
   (i) the genetically modified plant part is a non-regenerable plant part; or
   (ii) the genetically modified plant cell is a non-regenerable plant cell.
122. The genetically modified plant, plant part, or plant cell of any one of embodiments 118-121, wherein the plant part is a flower, a pistil, a leaf, a stem, a petiole, a cutting, a tissue, a seed coat, an ovule, pollen, a root, a rootstock, a scion, a fruit, a cotyledon, a hypocotyl, a protoplast, an embryo, an anther, or a portion thereof.
123. A processed plant product derived from the genetically modified plant, plant part, or plant cell of any one of embodiments 118-122 wherein the processed plant product comprises a detectable amount of the one or more Mime alleles of the genetically modified plant, plant part, or plant cell.
124. The processed plant product of embodiment 123, wherein the processed plant product:
   (i) is selected from the group consisting of plant biomass, oil, meal, food starch, syrup, animal feed, flour, flakes, bran, lint, hulls, and processed seed; and/or
   (ii) is non-regenerable.
125. Germplasm of the population of polyploid seeds of any one of embodiments 89-98.
126. A method of producing comprising a subpopulation of genetically uniform polyploid seed in an amount of at least 50% of the total number of seeds, the genetically uniform polyploid seed comprising three or more haplotypes of the same or related species of plant, wherein the population was obtained from a single plant or a set of F1 hybrids, the method comprising:
   (1)
      (a) generating a first parent Mime plant and a second parent MiMe plant;
      (b) providing clonal gametes from the first parent MiMe plant and the second parent MiMe plant that together comprise the three or more haplotypes; and
      (c) crossing the clonal gametes to produce the population of polyploid seed, or
   (2)
      (a) generating a first parent Mime plant;
      (b) providing clonal gametes from the parent MiMe plant;
      (c) providing haploid gametes from a homozygous parent non-MiMe plant; and
      (d) crossing the clonal gametes with the haploid gametes to produce the population of polyploid seed, wherein the generating in step (a) comprises introducing the following MiMe alleles (A) or (B) at the following MiMe loci by (i) introducing genetic modifications in the MiMe loci and selecting for lack of expression at the Mime loci, (ii) introducing an expression construct for a silencing RNA targeting the MiMe loci, or (iii) introducing a natural Mime allele or selecting a first parent Mime plant, a second parent MiMe plant or a progenitor with the natural allele for use in combination with (i) and/or (ii) for other Mime alleles; wherein genetic modifications and/or expression constructs are introduced into one or more candidate lines to produce MiMe alleles in germplasm of a first parent MiMe plant, a second parent MiMe plant, and/or progenitors thereof, wherein the first parent MiMe plant and the second parent MiMe plant each comprise a complete MiMe genotype, and each of the progenitors comprises a partial MiMe genotype, wherein if progenitors are generated, the progenitors are further crossed to generate the first parent Mime plant, the second parent MiMe plant, or both:
      (A) (1) one or more Mime alleles at one or more MiMe loci of a component of sister chromatid cohesion during the first division of meiosis comprising REC8 (i.e., a gene encoding a protein having at least 60% sequence identity to one of NP 001105829.1, XP 008648327.1, XP 008648329.1, and XP 008648328.1 or to XP 006347252.1), SWITCH1/DYAD (i.e., a gene encoding a protein having at least 60% sequence identity to one of NP_001139538.1, XP 008662288.1 and C0RWW9 or to M1BMI9), or a combination thereof, (2) one or more MiMe alleles at one or more MiMe loci of a component of DNA double strand breakage during meiotic recombination comprising PAIR1 (i.e., a gene encoding a protein having at least 60% sequence identity to one of XP 008660580.1, A0A1D6JK92, A0A1D6JK93, A0A1D6JK94, and A0A1D6PM18 or to one of XP 006339791.2 and M0ZGU5), SPO11-1 (i.e., a gene encoding a protein having at least 60% sequence identity to one of NP 001347894.1, XP 008643457.1, XP 008643458.1, XP 008643459.1, XP 020408860.1, and XP 008643458.1 or to one of XP 006346146.1, M1C0B8, and M1CP72), SPO11-2 (i.e., a gene encoding a protein having at least 60% sequence identity to one of XP 020406911.1, NP_001298099.1 and NP_001141583.1 or to one of XP 006344018.1, XP 006367265.1 and M1CP72), PRD1 (i.e., a gene encoding a protein having at least 60% sequence identity to one of XP 020399554.1, XP 020399553.1 and A0A1D6P5Q4 or to one of XP 015163123.1 and M1CA99), PRD2 (i.e., a gene encoding a protein having at least 60% sequence identity to one of NP_001130070.1, XP 035822500.1 and A0A3L6F902 or to one of XP 015162530.1 and M1AS84), DFO (i.e., a gene encoding a protein having at least 60% sequence identity to one of XP 020397706.1, A0A1D6QG87 and A0A3L6EXP8 or to one of XP 006350870.1 and M1CZR2), MTOPVIB (i.e., a gene encoding a protein having at least 60% sequence identity to one of XP 008645058.1, A0A3L6EMU7 and A0A1D6GVU9 or to one of XP 015166906.1, XP 015166907.1 and M1CGP5), DSY1, SY1, SY2, SY3, SY4, or any combination thereof, and (3) a natural os allele or one or more MiMe alleles at one or more MiMe loci of the component of progression through the second division of meiosis comprising OSD1 (i.e., a gene encoding a protein having at least 60% sequence identity to one of A0A1D6HBV9, A0A1D6HBW0, B4FCP3, B4FG75, and B6T7U1 or to one of XP 006351336.1), CYCA1 (i.e., a gene encoding a protein having at least 60% sequence identity to one of NP_001105387.2, NP_001288521.1 and XP 008656316.2 or to one of XP 006351137.1, XP 006351136.1 and XP 006351138.1), TDM1 (i.e., a gene encoding a protein having at least 60% sequence identity to one of NP_001170740.1, NP_001141607.1 and XP 008651103.1 or to one of XP 006350746.1, XP 006340757.1 and XP 006360343.1), PC1, PC2, FC, or any combination thereof; or
      (B) (2) one or more MiMe alleles at one or more MiMe loci of the component of DNA double strand breakage during meiotic recombination comprising PAIR1 (i.e., a gene encoding a protein having at least 60% sequence identity to one of), SPO11-1 (i.e., a gene encoding a protein having at least 60% sequence identity to one of), SPO11-2, PRD1, PRD2, DFO, MTOPVIB, DSY1, SY1, SY2, SY3, SY4, or any combination thereof, and (4) a natural ps allele or one or more MiMe alleles at one or more MiMe loci of the component of progression through the first division of meiosis comprise PS1 (i.e., a gene encoding a protein having at least 60% sequence identity to one of NP_001348386.1, XP 035820777.1 and XP 008668271.1 or to one of XP 006351164.1, XP 006353632.1 and M1AJ10), JASON (i.e., a gene encoding a protein having at least 60% sequence identity to one of NP_001132267.1, XP 008647301.1 and NP_001130670.1 or to one of XP 006352001.1, XP 015160469.1 and M1CT79), or a combination thereof;
   wherein the subpopulation of genetically uniform polyploid seeds comprises (i) a complete MiMe genotype comprising only MiMe alleles at each of the one or more MiMe loci, (ii) a partial MiMe genotype comprising at least one MiMe allele and at least one non-MiMe allele at each of the one or more MiMe loci, or (iii) a partially complemented MiMe genotype comprising only MiMe alleles at a first MiMe locus of the one or more MiMe loci and at least one Mime allele and at least one non-MiMe allele at a second Mime locus of the one or more MiMe loci.
127. The method of embodiment 126, wherein the subpopulation of genetically uniform polyploid seed is triploid, tetraploid, pentaploid, hexaploid, heptaploid, or octoploid.
128. The method of embodiment 126 or 127, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.
129. The population of any one of embodiments 126-128, wherein the population of polyploid seed comprises the subpopulation of genetically uniform polyploid seed in an amount of at least 70%, at least 90%, at least 95%, at least 96%, at least 97%, at least 98%, or at least 99% of the total number of seeds.
130. The population of embodiment 126-129, wherein each pair of seeds in the subpopulation of genetically uniform polyploid seed has a pairwise identity of at least 95%, at least 96%, at least 97%, at least 98%, at least 99%, or 100% as measured by the Jaccard similarity coefficient.
131. The population of any one of embodiments 126-130, wherein the subpopulation of genetically uniform polyploid seed has a partially-complemented MiMe genotype comprising:
   (A)
      (a) only MiMe alleles at one or more MiMe loci of a first Mime component;
      (b) one or more Mime alleles and one or more non-MiMe alleles at a first Mime locus of a second MiMe component, and one or more MiMe alleles and one or more non-MiMe alleles at a second MiMe locus of the second MiMe component; and
      (c) either (i) only Mime alleles at one or more MiMe loci of a third Mime component, or (ii) one or more Mime alleles and one or more non-MiMe alleles at a first MiMe locus of the third MiMe component, and one or more MiMe alleles and one or more non-MiMe alleles at a second MiMe locus of the third MiMe component, wherein the first MiMe component, the second MiMe component, and the third MiMe component are selected from the group consisting of (1) the component of sister chromatid cohesion during the first division of meiosis, (2) the component of DNA double strand breakage during meiotic recombination, and (3) the component of progression through the second division of meiosis, and each of the first MiMe component, the second MiMe component, and the third MiMe component are different MiMe components;
   (B)
      (a) only MiMe alleles at one or more MiMe loci of a first Mime component; and
      (b) one or more Mime alleles and one or more non-MiMe alleles at a first Mime locus of a second MiMe component, and one or more MiMe alleles and one or more non-MiMe alleles at a second MiMe locus of the second MiMe component,
      wherein the first MiMe component and the second MiMe component are selected from the group consisting of (2) the component of DNA double strand breakage during meiotic recombination and (4) the component of progression through the first division of meiosis and each of the first MiMe component and the second MiMe component are different MiMe components; or
   (C)
      (a) only MiMe alleles at one or more MiMe loci of a first MiMe component, wherein the first MiMe component is the component of DNA double strand breakage during meiotic recombination;
      (b) one or more MiMe alleles and one or more non-MiMe alleles at one or more Mime loci of a second MiMe component;
      (c) one or more MiMe alleles and one or more non-MiMe alleles at one or more MiMe loci of a third MiMe component; and
      (d) one or more MiMe alleles and one or more non-MiMe alleles at one or more Mime loci of a fourth MiMe component,
      wherein the second MiMe component, the third MiMe component, and the fourth MiMe component are selected from the group consisting of (1) the component of sister chromatid cohesion during the first division of meiosis, (3) the component of progression through the second division of meiosis, and (4) the component of progression through the first division of meiosis, and each of the second MiMe component, the third MiMe component, and the fourth MiMe component are different MiMe components.
132. The method of any one of embodiments 126-131, wherein the subpopulation of genetically uniform polyploid seed comprises:
   (1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1, optionally wherein the population of polyploid seed is from a maize plant and the one or more OSD1 loci comprise OSD1-1 and OSD1-2
   (5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
   (12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
   (13) a complete MiMe genotype comprising (i) an os allele, wherein the subpopulation of genetically uniforms seed is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
   (14) a partial MiMe genotype comprising (i) an os allele, wherein the subpopulation of genetically uniforms seed is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO111-1;
   (15) a complete MiMe genotype comprising (i) a ps allele, wherein the subpopulation of genetically uniforms seed is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
   (16) a partial MiMe genotype comprising (i) a ps allele, wherein the subpopulation of genetically uniforms seed is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
   (17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
   (18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the Mime alleles at the SPO11-1 locus comprise one or more genetic modifications resulting in decreased expression of SPO11-1;
   (20) a partially complemented MiMe genotype comprising (i) a ps allele, wherein the subpopulation of genetically uniforms seed is heterozygous for the ps allele, (ii) an os allele, wherein the subpopulation of genetically uniforms seed is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
   (21) a partially complemented MiMe genotype comprising (i) an os allele, wherein the subpopulation of genetically uniforms seed is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.
133. The population of any one of embodiments 126-132, wherein the subpopulation of genetically uniform polyploid seed comprises one or more polynucleotide sequences selected from the group consisting of SEQ ID NOs: 448-466, 468-471, 474-491, and 493-496.
134. The population of any one of embodiments 126-133, wherein germination of a seed of the subpopulation of genetically uniform polyploid seed results in a sterile plant that produces inviable gametes, seedless fruit, or a combination thereof.
135. The population of any one of embodiments 126-134, wherein the population of polyploid seed is from a plant selected from the group consisting of potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, ashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussel sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine, mandarin, clementine, coffee, cola, hazelnut, saffron, melon, cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycress, green bean, bean, ground cherry, pine, pistachio nut, pea, turf grass, poplar, apricot, plum, prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.

### EXAMPLES

The presently disclosed subject matter will be better understood by reference to the following Examples, which are provided as exemplary of the inventions, and not by way of limitation.

### Example 1: General Methods

### GM: Methods

### GM1: Identifying Homologs of Genes Underlying MiMe-Like Phenotype Across Plant Species

Core genes involved in the conversion of meiosis to mitosis (mitosis instead of meiosis or MiMe) were identified through alignment with any of the canonical reference sequences for RECS. OSD1 (e.g., OSD1-1, OSD1-2, and/or OSD1-3 from maize), PAIR1 (e.g., PAIR1-1 and/or PAIR1-2 from maize), SPO11-1, JASON (e.g., JASON-1 and JASON-2 from maize), CYCA1 (also known as CYCLIN-A1 or as TARDY ASYNCHRONOUS MEIOSIS (TAM)), TDM1, PS1, and/or PS1-LIKE as shown in Table 9 (SEQ IDs 1-119). For example, core MiMe genes from various plant genomes were identified through alignment with the Arabidopsis thaliana and Oryza sativa sequences of REC8, OSD1, CYCA1, PAIR1, and SPO11-1 sequences shown in Table 9 (SEQ ID NOs: 2, 13, 22, 31, 37, 54, 58, 71, 96, 110, 111, and 112). Amino acid sequences of the protein isoforms predicted from open reading frames of these genes were aligned to the NCBI RefSeq (O'Leary et al. (2016) Reference sequence (RefSeq) database at NCBI: current status, taxonomic expansion, and functional annotation. Nucleic Acids Res. 44(D1): D733-D745), UniProtKB Swiss-Prot and TrEMBL (The UniProt Consortium (2019) Uniprot: a worldwide hub of protein knowledge, Nucleic Acids Res. 47(D1): D506-D515, https://doi.org/10.1093/nar/gky1049) protein databases as well as MAKER-derived annotations of the Vaccinium corymbosum cv Draper v1.0 (Marivi Colle et al. (2019) Haplotype phased genome and evolution of phytonutrient pathways of tetraploid blueberry. GigaScience., 8(3)) genome, Rubus occidentalis v3.0 (VanBuren et al. (2016) The genome of black raspberry (Rubus occidentalis). The Plant Journal. 87(6): 535-547.) genome, Citrullus lanatus cv 97103 v2.0 (Guo S et al. (2013) The draft genome of watermelon (Citrullus lanatus) and resequencing of 20 diverse accessions. Nature Genetics. 45:51-58) genome, and Citrullus lanatus cv Charleston Gray v1.0 genome (Wu S. et al. (2019) Genome of ‘Charleston Gray’, the principal American watermelon cultivar, and genetic characterization of 1,365 accessions in the U.S. National Plant Germplasm System watermelon collection. Plant Biotechnol J. 17(12): 2246-2258). These alignments were performed using default parameters (gap opening penalty=11, gap extension penalty=1, E-value=10, word size=3, max score=25, query filter=SEG, substitution matrix=BLOSUM 62) in Protein-Protein BLAST 2.11.0+(O'Leary et al. (2016)). For example, core MiMe genes in a plant's genome may be identified through alignment with the REC8, OSD1, PAIR1, and SPO11-1 sequences from Arabidopsis thaliana and Oryza sativa as shown in Table 9 (SEQ ID NOs: 2, 13, 22, 31, 37, 54, 58, and 71).

Candidate orthologs were further validated through alignments between the identified orthologs and canonical protein sequences in Table 9 using Clustal Omega—1.2.4 with default parameters (substitution matrix=GONNET) for protein-protein alignments. The canonical protein sequences used were those from Arabidopsis thaliana for dicots, and those from Oryza sativa for monocots. In some cases, the canonical protein sequences available were from Arabidopsis thaliana. Results were filtered for relevant species and were further manually curated to remove spurious alignments. Orthologs with an amino acid consensus greater than 20% when aligning O. sativa queries against monocot (class Liliopsida) protein databases and A. thaliana against dicot (class Magnoliopsida) protein databases were considered as candidate orthologs with a preference for the highest conservation of amino acid sequence and exon structure relative to the canonical set (SEQ ID NOs: 1-87, 96, 110, 111, and 112) shown in Table 9.

GM1.1: Identification of Target DNA Sequences for crRNA Design

The protein Blast results were aligned to a genomic database using BLAST's “tblastn” search, specifically the Protein Query-Translated Subject BLAST 2.11.0+ with default parameters (gap opening penalty=11, gap extension penalty=1, E-value=10, word size=3, max score=25, query filter=SEG, query genetic code=universal, substitution matrix=BLOSUM 62). The corresponding nucleotide sequences of identified orthologs and any putative paralogs were extracted from this search, including 5 kb upstream of each gene, using custom shell scripts. These sequences were then aligned to each other using Clustal Omega—1.2.4 with default parameters (substitution matrix=GONNET). Most likely candidate sequences, with the highest identity to the canonical sequences and with the most conserved exon structure were used to design CRISPR RNA (crRNA) for an appropriate CRISPR-associated (Cas) nuclease.

GM1.2: Design of crRNA for DNA Editing with a Cas Nuclease

For each species of interest, the most probable candidate sequences identified from the protein BLAST, tblastn, and Clustal Omega workflows were targeted for crRNA design in Geneious Prime 2020.0.3, with protospacer adjacent motif (PAM) sites near each candidate identified for an appropriate Cas nuclease. crRNA with high specificity targeting the first or second exons or promoter sequences were generally preferred, but high-scoring crRNA targeting later exons were also selected. Resulting sequences were exported and scaffolds for an appropriate Cas nuclease were added. Functional crRNAs were synthesized by IDT (Integrated DNA Technologies, Newark, NJ, USA) using standard RNA synthesis. The process was repeated for each target gene, including REC8, OSD1 (e.g., OSD1-1, OSD1-2, and/or OSD1-3 from maize), PAIR1 (e.g., PAIR1-1 and/or PAIR1-2 from maize), SPO11-1, JASON (e.g., JASON-1 and JASON-2 from maize), CYCA1 (e.g., CYCA1-1 and CYCA1-2 from maize, or CYCA1-1, CYCA1-2 and CYCA1-3 from potato), TDM1, PS1, and/or PS1-LIKE. crRNA were screened for editing efficiency in protoplasts.

In monocots, editing was achieved by stable integration of two distinct DNA cassettes, one of which contained an appropriate codon-optimized Cas nuclease (i.e., the nuclease construct) and the other of which contained an array of crRNAs targeting MiMe alleles (i.e., the guide RNA construct). The DNA sequences corresponding to the crRNAs were inserted into a standard crRNA guide array between repeats, which were recognized and cleaved by either an appropriate ribonuclease or a ribozyme. The cassettes were each driven by their own plant ubiquitin promoter; however, plant U6 promoters or other suitable promoters with sufficient expression to achieve editing in the target plant were also tested and achieved similar results.

### GM2: Ribonucleoprotein Preparation

To prepare ribonucleoproteins (RNPs), 2 μL of New England Biolabs buffer (NEBuffer™) 2.1 (10× stock) was placed into a 1.5 mL microcentrifuge tube with 10-600 pmol of crRNA and with an equal amount of the selected Cas nuclease protein. The final volume was adjusted to 20 μL using nuclease-free water. The solution was prepared fresh and used after a 15-minute incubation at room temperature.

### GM3: Sequence-Based Edit Confirmation

Primers were designed to amplify each gene, and protoplasts were generated. Further details on plant selection, plant growth, protoplast generation, and protoplast transfection, are provided in the species-specific protocols in the Examples below.

Some transfected protoplasts were incubated at room temperature for 24 to 48 hours, then lysed and one or more long-range direct polymerase chain reactions (PCRs) were performed on the crude lysates. Other transfected protoplasts were regenerated, and DNA was then extracted from these protoplasts' regenerated callus, leaf, or other plant tissue. PCR products were pooled by transfection sample and a seqWell™ (Beverly, MA, USA) library preparation was performed to generate an Illumina (Illumina, San Diego, CA, USA) library. Samples were loaded onto an Illumina iSeq (Illumina, San Diego, CA, USA) and sequenced with a paired-end 150 nt sequencing kit. Sequences were analyzed by aligning FASTQ files to reference sequences and mutations adjacent to target sites for each gene were tabulated relative to a control. Editing efficiency was calculated based on the frequency of observed mutations in the reads obtained for a given sample, and this information was used to calculate how many plants should be screened to identify the multi-gene knockouts required to induce the clonal gamete production.

PCR amplicons were used to prepare Illumina sequencing libraries using plexWell 96 kits (seqWell™, Beverly, MA, USA) and libraries were sequenced on an Illumina iSeq (Illumina, San Diego, CA, USA). FASTQ data sets were aligned to the corresponding reference genomes using the BWA-MEM algorithm (Li, H. (2013) Aligning sequence reads, clone sequences and assembly contigs with BWA-MEM. arXiv preprint arXiv:1303.3997) and variants were visualized and quantified using custom scripts. Editing efficiency was calculated as the fraction of reads in an edited sample with on-target mutations relative to an unedited control. Editing efficiency was determined for each guide and used to estimate the minimum number of plants needed to recover the required multi-gene knockout.

### GM4: Flow Cytometry

Flow cytometry was performed on parent plants and on the progeny using the methods of Galbraith et al. 1983 (Rapid flow cytometric analysis of the cell cycle in intact plant tissues. Science. 220(4601): 1049-1051). Further details on parent plant selection and methods of crossing are provided in the species-specific protocols in the Examples below. Briefly, intact nuclei were extracted, filtered, and stained with propidium iodide as per instructions of the CyStain® PI Absolute P kit (Sysmex America, Lincolnshire, IL, USA). DNA content of nuclei was determined by applying the samples to a BD Accuri C6 Flow Cytometer. Gating was performed and genomic DNA content was calculated by comparing the peak area for the sample to the known position of a control with known ploidy. The ploidy of the unknown samples was determined based on relative comparison to each control.

### GM5: Analysis of Genetic Uniformity Using the Jaccard Similarity Coefficient

The Jaccard similarity coefficient was used to measure genetic uniformity of populations of seeds. The Jaccard index, Jaccard similarity index, or Jaccard similarity coefficient (Jaccard, P. (1908) Nouvelles Recherches sur la Distribution Florale. Bulletin de la Société Vaudoise des Sciences Naturelles. Vol. 44) is a metric used to compare the similarity of two sets. In the context of molecular plant genetics, the Jaccard index, Jaccard similarity index, or Jaccard similarity coefficient, is commonly applied to quantify the pairwise genetic similarity or uniformity of plants based on the presence or absence of shared alleles at loci spread throughout the genome (Paz and Veilleux (1997). Genetic diversity based on randomly amplified polymorphic DNA (RAPD) and its relationship with the performance of diploid potato hybrids. Journal of the American Society for Horticultural Sci. 122(6): 740-747; Vosman et al. (2004). The establishment of ‘essential derivation’ among rose varieties, using AFLP. Theoretical and Applied Genetics. 109: 1718-1725; Noli et al. (2013). Criteria for the definition of similarity thresholds for identifying essentially derived varieties. Plant Breeding. 132(6): 525-531; Vijayakumar et al. (2021) High temperature induced changes in quality and yield parameters of tomato (Solanum lycopersicum L.) and similarity coefficients among genotypes using SSR markers. Heliyon. 7(2); Dalamu et al. (2023). Genetic Diversity and Population Structure Analyses Using Simple Sequence Repeat Markers and Phenotypic Traits in Native Potato Collection in India. Potato Research: 1-25).

The Jaccard similarity coefficient is defined as the ratio of the number of shared items to the total number of distinct items in the two sets. In the context of molecular plant genetics, it can be used to quantify the proportion of shared alleles between two plants.

The formula for calculating the Jaccard similarity coefficient is:

$$J ( A , B ) = \vert A ⋂ B \vert / \vert A ⋃ B \vert$$

Where A represents the set of unique alleles without duplication in one plant, B represents the set of unique alleles without duplication in the other plant, |A∩B| represents the number of shared alleles (the cardinality of the intersection) between the plants, and |A∪B| represents the number of distinct alleles (the cardinality of the union) between the plants. This formula computes the cardinality of the intersection (common elements) of two sets (the shared alleles) divided by the cardinality of the union (all alleles) of the two sets (all distinct alleles present). The resulting value of the Jaccard similarity coefficient ranges from 0 to 1, where 0 indicates no shared alleles, and 1 indicates complete uniformity. The average pairwise genetic uniformity of the populations of seed was calculated as the average Jaccard similarity coefficient of all possible pairs of plants within the population. In the context of genetic pairwise similarity estimations, the size of A should be the same as, or very close to, the size of B to avoid misinterpretation.

Genotyping results found in the Examples below (such as in Example 2's section PR5, Example 5's section ZR4, and Example 7's section AR5) were encoded so that each allele at each locus represents a unique member of the set of total alleles. The number of shared alleles between each pair of progeny were divided by the total number of distinct alleles present in the two plants, and the results are the Jaccard index, Jaccard similarity index, or Jaccard similarity coefficient.

### GM6: Population Genotyping

All relevant parent and progeny plants were sequenced via whole genome shotgun (WGS) sequencing. DNA samples were sequenced by Novogene. Libraries were prepared using the NEBNextR Ultra™ II for DNA Library Prep kit, and paired-end 2×150 reads were sequenced on an Illumina NovaSeq 6000 or NovaSeq X plus. Germline SNPs and small indels were called in all relevant parents using a standard whole genome variant calling pipeline. WGS data was aligned to a reference genome with BWA-MEM (Li, H. (2013) Aligning sequence reads, clone sequences and assembly contigs with BWA-MEM. arXiv preprint arXiv:1303.3997). PCR duplicates were removed and SNPs and small indels were called and jointly genotyped with GATK (McKenna, A et al. (2010) The Genome Analysis Toolkit: a MapReduce framework for analyzing next-generation DNA sequencing data. Genome research, 20(9), pp. 1297-1303). Tri-allelic and tetra-allelic markers were established to genotype populations.

Tri-allelic (containing three alleles) markers were used when one parent was expected to contribute one distinct haplotype while the other parent was expected to contribute two distinct haplotypes. Pairs of SNPs that were close enough to be directly phased by 150 bp Illumina reads were used. Each SNP was unique/exclusive to one of the parents. One SNP/parent was homozygous while the other was heterozygous (REF/ALT or ALT1/ALT2). To genotype progeny, the inventors looked for the presence of both ALT alleles (one from each parent) as well as the presence of the reference allele or second alternate allele of the heterozygous SNPs. If all alleles were observed and phasing was consistent with parental haplotypes, then the locus was genotyped as tri-allelic.

Tetra-allelic (containing four alleles) markers were used when both parents were expected to contribute two distinct haplotypes. Trios of SNPs that were close enough to be directly phased by 150 bp Illumina reads were used. One SNP was unique/exclusive to one parent and the two other SNPs were unique/exclusive to the other parent. All SNPs were heterozygous. The ALT alleles of the two SNPs exclusive to the same parent were on opposing haplotypes. To genotype progeny, the inventors looked for the presence of all three ALT alleles (one from one parent and two from the other) as well as the presence of the reference allele or second alternate allele of the solo heterozygous SNPs. If all alleles were observed and phasing was consistent with parental haplotypes, then the locus was genotyped as tetra-allelic.

After establishing markers, markers were filtered, genotyped, and checked for quality across all progenies (via WGS data) using a combination of manual and custom automated techniques.

### Example 2: Generating Genetically Uniform Tetraploid Hybrid Potato

### PM: Methods

### PM1: Plant Materials and Protoplast Isolation

Plants from potato accessions that were selected and tested included F1 hybrids between the doubled monoploid DM 1-3 516 R44 (Solanum tuberosum Group Phureja, also referred to as “DM1-3” or “DM”) and the diploid S7 inbred M6 (Solanum chacoense); F1 hybrids between the doubled monoploid DM 1-3 516 R44 (Solanum tuberosum Group Phureja) and M18 (the diploid S5 inbred diploid derived from the dihaploid S. tuberosum ssp. tuberosum clone US-W4); and dihaploid Solanum tuberosum ssp. tuberosum lines derived from the tetraploid commercial cultivar Atlantic.

The plant material was propagated in vitro via internode cuttings on Murashige & Skoog Modified BC Potato Medium containing sucrose and grown under 16-hour day with cool white fluorescent lighting. Approximately one gram of leaves from 2-3 week old explants were removed under aseptic conditions and placed in sterile water. Other diploid Solanum spp. were also selected and propagated in the same manner.

Leaves were sliced into thin sections approximately 1 mm in width and incubated in digest solution overnight. (See, e.g., Clasen et al. (2016). Improving cold storage and processing traits in potato through targeted gene knockout. Plant biotechnology journal, 14(1), 169-176; Fossi et al. (2019). Regeneration of Solanum tuberosum plants from protoplasts induces widespread genome instability. Plant physiology, 180(1), 78-86; Masson et al. (1987). Plant regeneration from protoplasts of diploid potato derived from crosses of Solanum tuberosum with wild Solanum species. Plant Science, 53(2), 167-176; Nicolia et al. (Nicolia et al. (2015) Targeted gene mutation in tetraploid potato through transient TALEN expression in protoplasts. Journal of biotechnology. 204: 17-24; Veillet et al. (2019). The Solanum tuberosum GBSSI gene: a target for assessing gene and base editing in tetraploid potato. Plant cell reports, 38(9), 1065-1080). Sliced leaves in the digest solution were then incubated overnight at 24° C. The next day, protoplasts were liberated from the leaf tissue with 15-minute shaking at 40 RPM at room temperature.

Protoplasts were harvested through 100 μm sterile cell filters into sterile 50 mL conical tubes and centrifuged at 100×g for 5 minutes. Supernatant was removed and replaced with wash solution (See, e.g., Clasen et al. 2016; Fossi et al. 2019; Masson et al. 1987; Nicolia et al. 2015; Veillet et al. 2019). Cells were then gently resuspended by rocking and slowly layered onto a 0.43 M sucrose solution. Tubes were centrifuged at 100×g for 15 minutes. After 15 minutes, a thick dark band of protoplasts appeared at the interface of the two solutions. This band was harvested in one continuous motion using a sterile serological pipette and combined with transformation buffer (See, e.g., Clasen et al. 2016; Fossi et al. 2019; Masson et al. 1987; Nicolia et al 2015; Veillet et al. 2019). Harvested cells were quantified using a Bürker hemocytometer and stored at 4° C. in the dark until transfection. A sample was reserved to test cell viability using FDA staining as described by Larkin (1976. Purification and viability determinations of plant protoplasts. Planta. 128(3): 213-216).

### PM2: Protoplast Transfection

Transfection proceeded as described in the art. (See, e.g., Clasen et al. 2016; Fossi et al. 2019; Masson et al. 1987; Nicolia et al 2015; Veillet et al. 2019). Protoplasts were centrifuged at 50×g for 10 minutes and resuspended in a volume of transformation buffer to achieve a cell density of 1×10⁶ protoplasts/mL. 20 μL of freshly prepared RNP as described in section GM2 was added to the bottom of a 15 mL round bottom tube and 100 μL of protoplasts suspended in the transformation buffer were mixed with the RNP solution. Next, 120 μL of PEG solution was added and gently mixed by rotating the tubes. After a 15-minute incubation at room temperature the protoplasts were washed twice following centrifugation at 50×g for 10 minutes using a wash solution consisting of 0.4M D-Mannitol, 15 mM CaCl₂, and 5 mM HEPES (4-(2-hydroxyethyl)-1-piperazineethanesulfonic acid). A final centrifugation was performed and transfected cells were resuspended in culture medium. (See, e.g., Clasen et al. 2016; Fossi et al. 2019; Masson et al. 1987; Nicolia et al 2015; Veillet et al. 2019).

The protoplasts were transfected with RNPs designed to target MiMe loci identified as described in GM1 and prepared as described in GM2

### PM3: Encapsulation and Regeneration of Plants

An equal volume of transfected cells was combined with a 3.2% sodium alginate solution and then gently mixed together. The resulting solution was pipetted on top of setting agar to solidify the alginate matrix, creating alginate lenses. Lenses were incubated for 30 minutes at room temperature and then transferred to a new petri dish with 20 mL of culture medium.

Plant regeneration protocols proceeded as described in the art. (See, e.g., Clasen et al. 2016; Fossi et al. 2019; Masson et al. 1987; Nicolia et al 2015; Veillet et al. 2019). The alginate lenses in culture medium were placed at 24° C. in the dark for about 3-4 weeks, during which time cell division and signs of mini calli induction were observed through an inverted microscope. When mini-calli reached 1 mm in size, culture medium was replaced with a first callus induction medium as described in the art (see, e.g., Clasen et al. 2016; Fossi et al. 2019; Masson et al. 1987; Nicolia et al 2015; Veillet et al. 2019) and light was gradually increased by first covering and then removing layers of cheesecloth under cool white fluorescent bulbs. After 3-6 weeks, or when minicalli reach approximately 2 mm in diameter, the mini calli were liberated from the alginate using a citric acid solution consisting of 20 mM sodium citrate and 0.5 M sorbitol. Mini calli were then washed in callus induction medium and incubated in a second callus induction medium, then replacing the second callus induction medium with a freshly prepared solution on a weekly basis. (See, e.g., Clasen et al. 2016; Fossi et al. 2019; Masson et al. 1987; Nicolia et al 2015; Veillet et al. 2019). Finally, large green calli of 3-5 mm were removed and placed on Petri dishes with shoot induction media. (See, e.g., Clasen et al. 2016; Fossi et al. 2019; Masson et al. 1987; Nicolia et al 2015; Veillet et al. 2019). Calli were then transferred every 2 weeks to a fresh shoot induction medium until shoots emerged, in about 2-3 months. When shoots reached a size of approximately 1 cm on shoot induction media, they were excised from the calli and transferred to Murashige & Skoog Modified BC Potato Medium with sucrose and 16-hour day length under cool white fluorescent bulbs.

Plants were then propagated by nodal segments and genotyped using the same sequencing methods described in Example 1's section GM3. Plants containing bi-allelic mutations in target MiMe genes were further multiplied and prepared for planting.

### PM4: Plant Growth and Crossing Conditions

Once plants had completely regenerated in vitro from protoplasts, rooted plantlets were transferred from tissue culture into 1-gallon pots with peat substrate and grown under greenhouse conditions with a continued 16-hour daylength. Freshly transplanted in vitro plants were covered with humidity domes for up to 3 days.

Regenerated plants with confirmed edits in each target gene, including REC8, CYCA1, and PAIR1 and/or SPO11, were transplanted from tissue culture into peat substrate as described in the previous paragraph. When plants produced flowers, pollen was collected from the male and cross pollinations were performed. About 4-6 weeks after pollination, berries were harvested and potato true seeds were extracted, dried, and prepared for planting.

### PM5: Potato True Seed Germination and Genotyping

Following extraction, potato true seeds were surface disinfected for 20 minutes in a sonic bath using a ten percent bleach solution containing 20 μL of Tween 20 (Sigma) in a final volume of 50 mL. Disinfected potato true seeds were rinsed twice with sterile deionized water and were germinated in Murashige & Skoog (M&S) Modified BC Potato Medium with sucrose and 16-hour day length under cool white fluorescent bulbs. For in vitro germination, berries were surface disinfected and sliced open under aseptic conditions. Embryos were removed carefully and plated directly. Plants were transferred every two weeks to fresh medium and leaves were removed for DNA extraction during this process.

High-quality genomic DNA was extracted from the leaves of randomly selected plants of each population and used for genotyping through Qiagen DNeasy Plant Mini kits (Cat. No. 69104) (QIAGEN, Germantown, MD, USA). Purity was determined using a spectrophotometer, Nanodrop™ One-C (ThermoFisher, Waltham, MA, USA), and DNA was quantified on a fluorometer, Qubit™ Flex Fluorometer (Cat. No. Q33327) (ThermoFisher, Waltham, MA, USA). Samples were adjusted to a final concentration of 20 ng/μL. Markers were established and populations were genotyped as described in GM6.

### PR: Results

### PR1: Guide RNA Screening

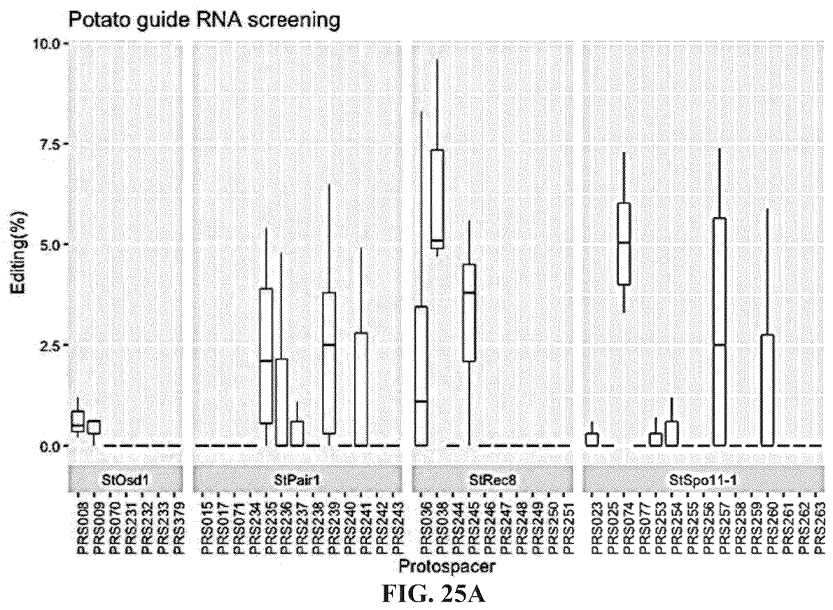

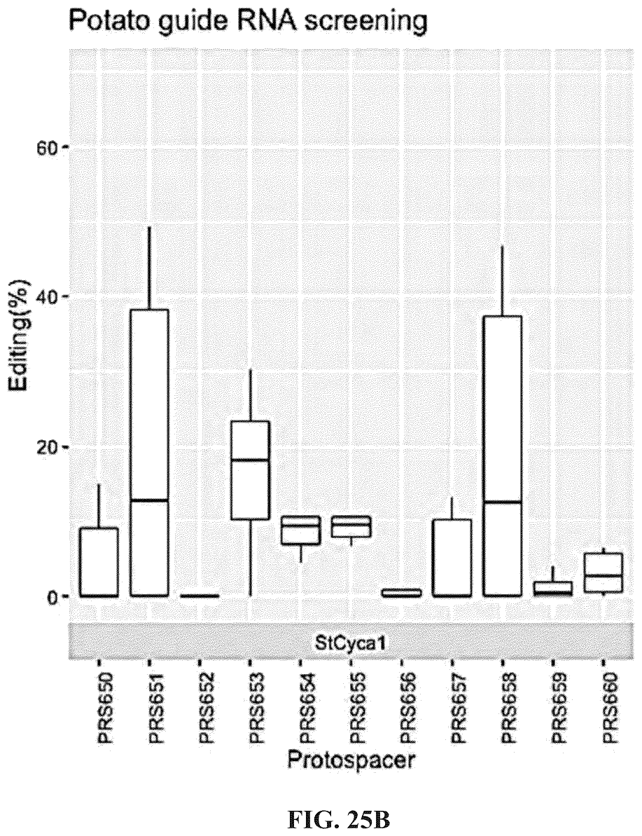

A total of 57 guide RNAs (SEQ ID NOs: 120-174, shown in Table 11A) targeting conserved regions of the OSD1, CYCA1, SPO11-1, REC8, or PAIR1 genes provided by SEQ ID NOs: 175-229, shown in Table 11B, were transfected in triplicate in protoplasts from the Solanum tuberosum or Solanum chacoense accessions described in section PM1. Editing efficiency was calculated by sequencing as described in Example 1's section GM3, and the resulting data presented in FIG. 25A and FIG. 25B. Based on this data, guide RNAs SEQ ID NO: 153 and SEQ ID NO: 155 were chosen for editing the OSD1 gene; guide RNA SEQ ID NO: 172 was chosen for editing the CYCA1 gene; guide RNAs SEQ ID NO: 150, SEQ ID NO: 142 and SEQ ID NO: 132 were chosen for editing the PAIR1 gene; guide RNA SEQ ID NO: 136 was chosen for editing the REC8 gene; and guide RNA SEQ ID NO: 134 was chosen for editing the SPO11-1 gene.

TABLE 11A
Sequences for guide RNAs
targeting MiMe genes in potato.

| SEQ ID NO. | PRSG# | Guide RNA sequence |
|---|---|---|
| 120 | PRS234G | CUUUCAGUGGAAAGCUUACG |
| 121 | PRS242G | CUCUUUCUUGAGAACCCAAU |
| 122 | PRS259G | GCACUCUAUAAUGCAGAACA |
| 123 | PRS235G | AAGGUCCCCUCCAGGCUAGC |
| 124 | PRS243G | CCACAUCACGUAAAUUAAAA |
| 125 | PRS260G | AUGGAAGCGCUAUCUACCUU |
| 126 | PRS236G | AAUAUGCAACAUGCCUGAAU |
| 127 | PRS253G | AACGCUUUCCCUCCAUUUUC |
| 128 | PRS261G | GCAGGAUUUACUCGUUCCAU |
| 129 | PRS254G | GUUGUGGAAAAGGAAUCAGG |
| 130 | PRS262G | UGUAGCUCUUGCAGCUCUGG |
| 131 | PRS255G | AGUUGUGGAAAAGGAAUCAG |
| 132 | PRS239G | AGCAAGACCUUGGCUAAAGA |
| 133 | PRS256G | GUUGAGAGUUCUUGUGAUAG |
| 134 | PRS257G | CUCUCCCUAGACUCACCAAG |
| 135 | PRS258G | CCACAACUAAAACGUAUUGA |
| 136 | PRS036G | GAGUGUAGAGUGGCCGCCAU |
| 137 | PRS246G | AAGCAUGAAUUACUCUGCAG |
| 138 | PRS247G | CAGUUGAUCCAGAUAAUAUC |
| 139 | PRS248G | GGUUUAGAUAUCUAAUCGUC |
| 140 | PRS237G | AUAGUAUCUGCAAAUGAAGU |
| 141 | PRS249G | AAGUUAUUUCGUGGAUUGAG |
| 142 | PRS238G | UAGGUCAUCAAUGAUUGUAG |
| 143 | PRS263G | AAAGGUAAAUCUGGAUCGAC |
| 144 | PRS250G | CAUAAUGUACCAUCGUGAAA |
| 145 | PRS251G | CGAAGCAUACCCAUGAGAAU |
| 146 | PRS240G | ACCUUGCAUGAGGAGGAAGA |
| 147 | PRS244G | UGAUUUGGUAUUUUCAGGUG |
| 148 | PRS241G | GUGGAUCGACUGGAGCGGUU |
| 149 | PRS245G | UAUCUCUCUCUUUGCUGUGA |
| 150 | PRS071G | CCUCAUAUCAACUACACACGA |
| 151 | PRS070G | UCUCAAGCGAGCUCUUGUUCU |
| 152 | PRS038G | CUAUUGAGUUUCGAGUGUAG |
| 153 | PRS008G | GACGUGGGGUGAUCAAAGAA |
| 154 | PRS015G | AGUCUCUUCUCGUGUGUAGU |
| 155 | PRS009G | AUCACCCCACGUCCAAAAAC |
| 156 | PRS231G | GCUUUAUAUAGUUCUGGAUG |
| 157 | PRS232G | CUCCAUAGGCCAUUGAAAGA |
| 158 | PRS017G | CUUUCAGGAACUGACCAUUU |
| 159 | PRS233G | GCACUUUACCAACAGAUCUG |
| 160 | PRS379G | AUGUCAAUGCGCUGAUUUUC |
| 161 | PRS074G | CUUAUGAGUGUUAGUGGCUGC |
| 162 | PRS077G | CUCCCAGAUUCCACAUACCGC |
| 163 | PRS025G | UGAACCGAUCGAUGUAGAUC |
| 164 | PRS650G | UUGGCCGCUCCAGCAUUCAA |
| 165 | PRS651G | GAAGGCACCAUGUACGUCCA |
| 166 | PRS652G | AUAUGUUCUGAGAUGUAGAG |
| 167 | PRS653G | UUCCAGCAGCAGAUAUAUGC |
| 168 | PRS654G | UUAUGCUCCAUCACUCAUUG |
| 169 | PRS655G | ACAAUUAACUACAUUGAUCG |
| 170 | PRS656G | GGGCAACUUGAUGGACAGAC |
| 171 | PRS657G | UUACGAUGCGAUCAUCAUGC |
| 172 | PRS658G | CUCAGGGACUGCUGUUCUGC |
| 173 | PRS659G | UCUAUUUUUUAUUCCAGCAG |
| 174 | PRS660G | GAAACUCCUAACAGAACUCU |

TABLE 11B
Sequences for conserved regions
of MiMe genes targeted in potato.

| SEQ ID NO. | PRS# | Target DNA sequence |
|---|---|---|
| 175 | PRS234 | CTTTCAGTGGAAAGCTTACG |
| 176 | PRS242 | CTCTTTCTTGAGAACCCAAT |
| 177 | PRS259 | GCACTCTATAATGCAGAACA |
| 178 | PRS235 | AAGGTCCCCTCCAGGCTAGC |
| 179 | PRS243 | CCACATCACGTAAATTAAAA |
| 180 | PRS260 | ATGGAAGCGCTATCTACCTT |
| 181 | PRS236 | AATATGCAACATGCCTGAAT |
| 182 | PRS253 | AACGCTTTCCCTCCATTTTC |
| 183 | PRS261 | GCAGGATTTACTCGTTCCAT |
| 184 | PRS254 | GTTGTGGAAAAGGAATCAGG |
| 185 | PRS262 | TGTAGCTCTTGCAGCTCTGG |
| 186 | PRS255 | AGTTGTGGAAAAGGAATCAG |
| 187 | PRS239 | AGCAAGACCTTGGCTAAAGA |
| 188 | PRS256 | GTTGAGAGTTCTTGTGATAG |
| 189 | PRS257 | CTCTCCCTAGACTCACCAAG |
| 190 | PRS258 | CCACAACTAAAACGTATTGA |
| 191 | PRS036 | GAGTGTAGAGTGGCCGCCAT |
| 192 | PRS246 | AAGCATGAATTACTCTGCAG |
| 193 | PRS247 | CAGTTGATCCAGATAATATC |
| 194 | PRS248 | GGTTTAGATATCTAATCGTC |
| 195 | PRS237 | ATAGTATCTGCAAATGAAGT |
| 196 | PRS249 | AAGTTATTTCGTGGATTGAG |
| 197 | PRS238 | TAGGTCATCAATGATTGTAG |
| 198 | PRS263 | AAAGGTAAATCTGGATCGAC |
| 199 | PRS250 | CATAATGTACCATCGTGAAA |
| 200 | PRS251 | CGAAGCATACCCATGAGAAT |
| 201 | PRS240 | ACCTTGCATGAGGAGGAAGA |
| 202 | PRS244 | TGATTTGGTATTTTCAGGTG |
| 203 | PRS241 | GTGGATCGACTGGAGCGGTT |
| 204 | PRS245 | TATCTCTCTCTTTGCTGTGA |
| 205 | PRS071 | CCTCATATCAACTACACACGA |
| 206 | PRS070 | TCTCAAGCGAGCTCTTGTTCT |
| 207 | PRS038 | CTATTGAGTTTCGAGTGTAG |
| 208 | PRS008 | GACGTGGGGTGATCAAAGAA |
| 209 | PRS015 | AGTCTCTTCTCGTGTGTAGT |
| 210 | PRS009 | ATCACCCCACGTCCAAAAAC |
| 211 | PRS231 | GCTTTATATAGTTCTGGATG |
| 212 | PRS232 | CTCCATAGGCCATTGAAAGA |
| 213 | PRS017 | CTTTCAGGAACTGACCATTT |
| 214 | PRS233 | GCACTTTACCAACAGATCTG |
| 215 | PRS379 | ATGTCAATGCGCTGATTTTC |
| 216 | PRS074 | CTTATGAGTGTTAGTGGCTGC |
| 217 | PRS077 | CTCCCAGATTCCACATACCGC |
| 218 | PRS025 | TGAACCGATCGATGTAGATC |
| 219 | PRS650 | TTGGCCGCTCCAGCATTCAA |
| 220 | PRS651 | GAAGGCACCATGTACGTCCA |
| 221 | PRS652 | ATATGTTCTGAGATGTAGAG |
| 222 | PRS653 | TTCCAGCAGCAGATATATGC |
| 223 | PRS654 | TTATGCTCCATCACTCATTG |
| 224 | PRS655 | ACAATTAACTACATTGATCG |
| 225 | PRS656 | GGGCAACTTGATGGACAGAC |
| 226 | PRS657 | TTACGATGCGATCATCATGC |
| 227 | PRS658 | CTCAGGGACTGCTGTTCTGC |
| 228 | PRS659 | TCTATTTTTTATTCCAGCAG |
| 229 | PRS660 | GAAACTCCTAACAGAACTCT |

### PR2: Generation of MiMe Parent Plants

Regenerated plants were screened using the sequencing methods described in section GM3. About 1% of regenerated plants screened (roughly 200 plants out of 20,000 total plants) had mutations predicted to result in either nonfunctional or non-expressed MiMe alleles, resulting in a MiMe genotype. The screened plants with these mutations were selected for crosses. These mutations were predicted to result in (i) either nonfunctional or non-expressed rec8, cyca1, and spo11-1 alleles; (ii) either nonfunctional or non-expressed rec8 and spo11-1 alleles in combination with the os allele; or (iii) either nonfunctional or non-expressed spo11-1 allele in combination with the ps allele.

Select potato plants were given designations according to the following format. The first part of the designation, “PED-PR”, was used to indicate plants involved in the potato results. The second part of the designation uses capital letters to indicate the haplotypes the selected plants contain (see Table 12A). The third part of the designation uses lower case letters to indicate which edits or natural alleles were used in the selected plants (see Table 12B).

TABLE 12A
Letter codes in PED-PR designations to indicate
the haplotypes the selected plants contain.
Haplotype

| | |
|---|---|
| DM1-3 516 R44 | A |
| M6 | B |
| M18 selfed | C |
| M18 | D |
| Atlantic Dihaploid | E |
| Atlantic Dihaploid | F |

TABLE 12B
Letter codes in PED-PR designations to indicate which
edits or natural alleles were used in the selected plants.
Edits or natural alleles

| | |
|---|---|
| rec8 | r |
| cyca1 | c |
| spo11-1 | s |
| ps | p |
| os | o |

Two unique plants designated PED-PR-AB-res and PED-PR-EF-res were chosen to move forward as parent MiMe plants for crossing, each of which had bi-allelic edits resulting in a MiMe genotype for rec8, cyca1 and spo11-1, and each of which produced both male and female clonal gametes. The parent MiMe plant PED-PR-AB-res (rec8/cyca1/spo11-1) was derived from an F1 cross between DM 1-3 516 R44 and M6. The parent MiMe plant PED-PR-EF-res (rec8/cyca1/spo11-1) was derived from an Atlantic dihaploid.

Three additional unique plants designated PED-PR-EF-rso-2, PED-PR-AB-rso and PED-PR-EF-rso-1 were chosen to move forward as parent MiMe plants for crossing, each of which had bi-allelic edits resulting in a MiMe genotype for rec8 and spo11-1 in combination with the os allele. All three parent MiMe plants PED-PR-EF-rso-2 (rec8/spo11-1/os), PED-PR-AB-rso (rec8/spo11-1/os) and PED-PR-EF-rso-1 (rec8/spo11-1/os) produced male sterility and female clonal gametes. The parent MiMe plants PED-PR-EF-rso-2 (rec8/spo11-1/os) and PED-PR-EF-rso-1 (rec8/spo11-1/os) were each derived from an Atlantic dihaploid. The parent MiMe plant PED-PR-AB-rso (rec8/spo11-1/os) was derived from an F1 cross between DM1-3 516 R44 and M6.

Two unique plants designated PED-PR-AB-sp and PED-PR-AD-sp were chosen to move forward as parent MiMe plants for crossing, each of which had bi-allelic edits resulting in a MiMe genotype for spo11-1 in combination with the ps allele. Both parent MiMe plants produced male clonal gametes and female sterility. The parent MiMe plant PED-PR-AB-sp (spo11-1/ps) was derived from an F1 cross between DM 1-3 516 R44 and M6. The parent MiMe plant PED-PR-AD-sp (spo11-1/ps) was derived from an F1 cross between DM 1-3 516 R44 and M18.

Microscopic analysis of microsporogenesis showed that these parent MiMe plants produced dyads instead of tetrads and produced viable pollen (except in the male sterile plants). Plants with expected female clonal gametes were confirmed by test crosses with 1n and 2n pollen.

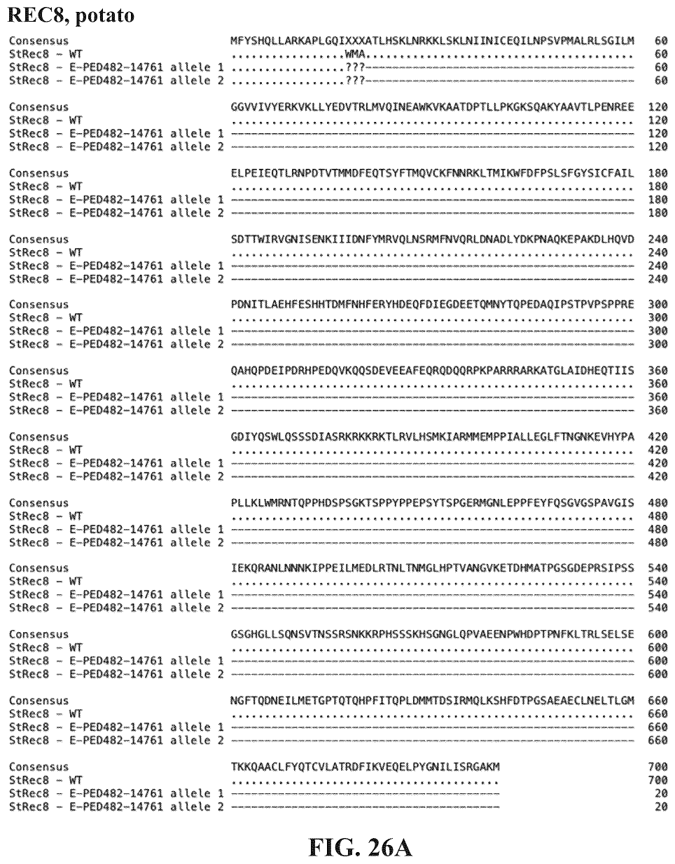

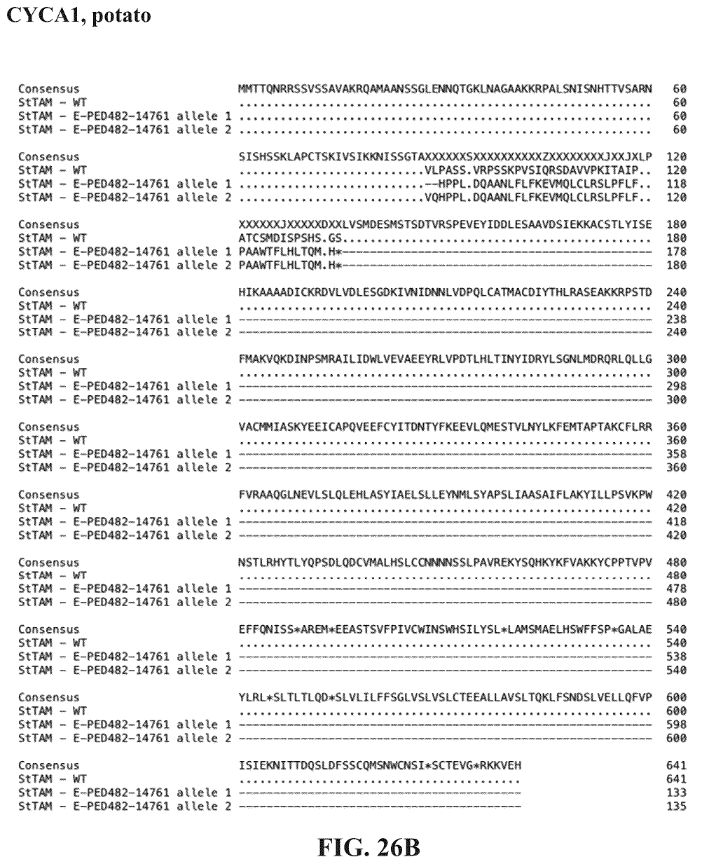

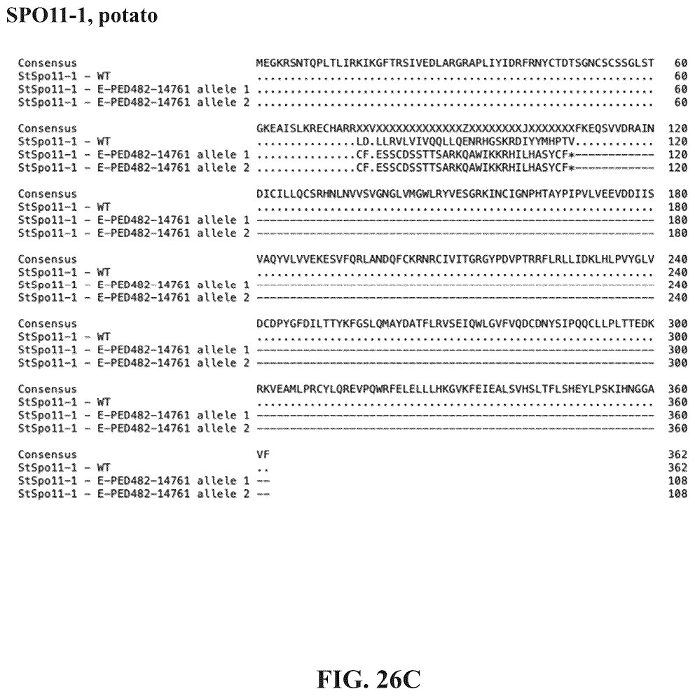

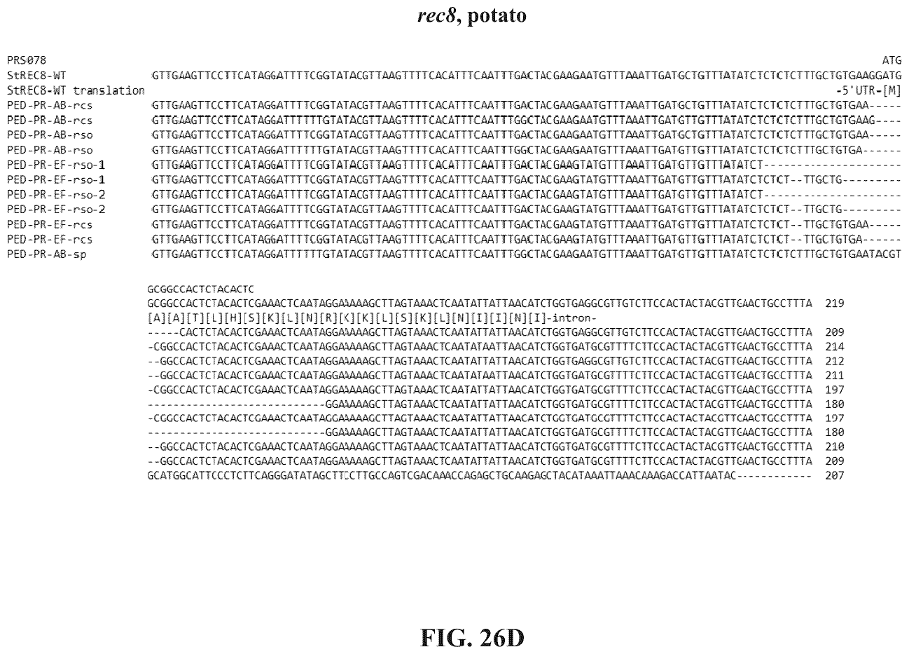

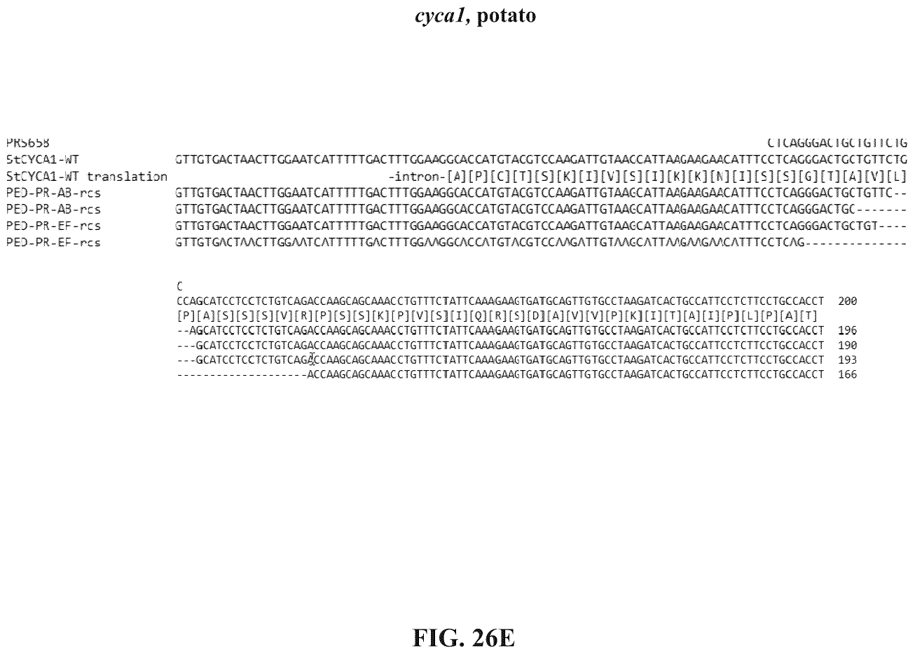

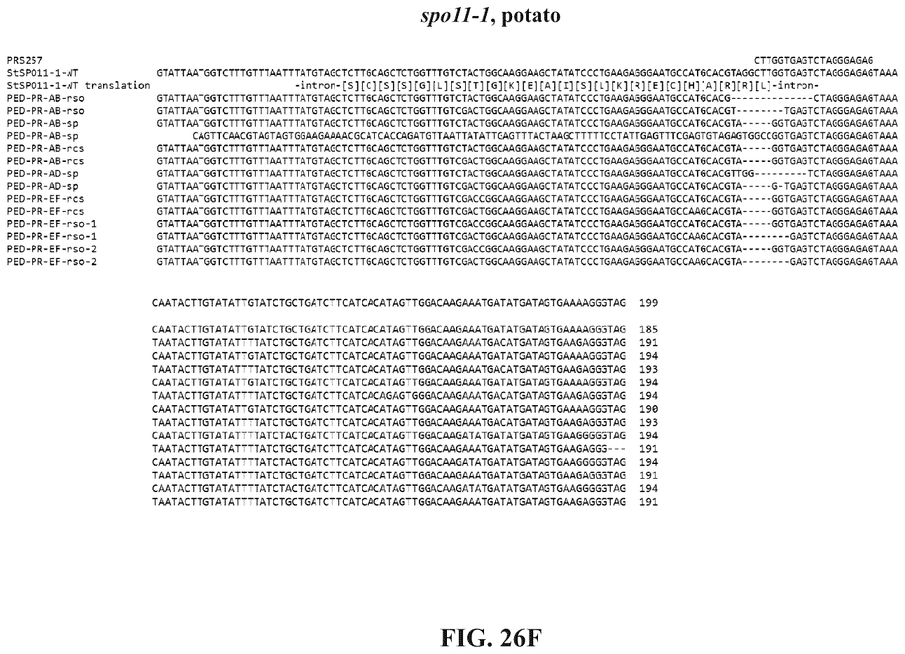

As an example, edits to the parent MiMe plant PED-PR-AB-res (rec8/cyca1/spo11-1) are illustrated in FIGS. 26A-26F. In that figure, the predicted protein sequences are illustrated with respect to the unedited wildtype protein sequence and a consensus sequence, wherein an “X” in the consensus sequence indicates that there is no consensus for that amino acid position; wherein a dot (“.”) in the parent MiMe plant sequence indicates that the respective amino acid matches the consensus sequence; and wherein a dash (“-”) in the parent MiMe plant sequence indicates that the respective amino acid is present in the consensus sequence but is absent in the parent MiMe plant sample sequence. Frameshift inducing deletions in both alleles of rec8 (FIGS. 26A and 26D), cyca1 (FIGS. 26B and 26E) and spo11-I (FIGS. 26C and 26F) result in premature stop codons. Premature stop codons are denoted by an asterisk (“*”) in FIGS. 26A-26C.

### PR2b: Generation of Control, Non-MiMe Parent Plants

Four unique plants designated PED-PR-EF-o, PED-PR-AABB, PED-PR-CC-o and PED-PR-AB, respectively, were chosen as parent non-MiMe plants for control crosses. PED-PR-EF-o was a dihaploid derived from the tetraploid S. tuberosum cultivar “Atlantic”. PED-PR-AABB was derived from a tetraploidized hybrid between the S. tuberosum Group Phureja doubled monoploid “DM1-3 516 R44” and the S7 inbred S. chacoense “M6”. PED-PR-CC-o was derived from a self of the S. tuberosum S5 inbred “M18”. M18 was derived from 5 generations of selfing of the S. tuberosum dihaploid “US-W4”. PED-PR-AB was derived from a hybrid between the S. tuberosum Group Phureja doubled monoploid “DM1-3 516 R44” and the S7 inbred S. chacoense “M6”.

### PR3a: Generation of Genetically Uniform Tetraploid Potato True Seed

Parent MiMe plants were crossed using hand pollination. Potato true seeds resulting from crosses were germinated following the procedures described in section PM5. In total, five genetically uniform populations of tetraploid potato true seed with either three or four haplotypes were created from the parent MiMe plants described in section PR2. The parent MiMe plants used in the crosses to generate the genetically uniform populations are outlined in Table 12C.

The first genetically uniform population of tetraploid potato true seed, referred to herein as Boosted Potato Population 1 (“BPP1”), consisted of 20 individuals with three haplotypes, derived from a cross between a female unedited selfed M18 potato plant PED-PR-CC-o and a male parent MiMe plant PED-PR-AB-sp (spo11-1/ps), which together comprise three haplotypes.

The second genetically uniform population of tetraploid potato true seed, referred to herein as Boosted Potato Population 2 (“BPP2”), consisted of 36 individuals with four haplotypes, derived from a cross between a female parent MiMe plant PED-PR-EF-rso-1 (rec8/spo11-1/os) and a male parent MiMe plant PED-PR-AB-sp (spo11-1/ps), which together comprise three haplotypes.

The third genetically uniform population of tetraploid potato true seed, referred to herein as Boosted Potato Population 3 (“BPP3”), consisted of 10 individuals with three haplotypes, derived from a cross between a female parent MiMe plant PED-PR-AB-rso (rec8/spo11-1/os) and a male parent MiMe plant PED-PR-AD-sp (spo11-1/ps), which together comprise three haplotypes.

The fourth genetically uniform population of tetraploid potato true seed, referred to herein as Boosted Potato Population 4 (“BPP4”), consisted of 12 individuals with four haplotypes, derived from a cross between a female parent MiMe plant PED-PR-EF-rso-2 (rec8/spo11-1/os) and a male parent MiMe plant PED-PR-AB-res expected to produce male and female clonal gametes (rec8/cyca1/spo11-1), which together comprise three haplotypes.

The fifth genetically uniform population of tetraploid potato true seed, referred to herein as Boosted Potato Population 5 (“BPP5”), consisted of 8 individuals with four haplotypes, derived from a cross between a female parent MiMe plant PED-PR-EF-res (rec8/cyca1/spo11-1) and a male parent Mime plant PED-PR-AB-res (rec8/cyca1/spo11-1), which together comprise three haplotypes.

### PR3b: Generation of Standard Tetraploid Potato True Seed

The first standard population of tetraploid potato true seed, referred to herein as Standard Potato Population 1 (“SPP1”), consisted of 8 individuals with four haplotypes, derived from a cross between a female parent non-MiMe plant PED-PR-EF-o and a male parent non-MiMe plant PED-PR-AABB.

The second standard population of diploid potato true seed, referred to herein as Standard Potato Population 2 (“SPP2”), consisted of 6 individuals with two haplotypes, derived from a cross between a female unedited selfed M18 PED-PR-CC-o and a male parent non-MiMe plant PED-PR-AB.

The parent plants used in the crosses to generate SPP1 and SPP2 are outlined in Table 12C.

TABLE 12C
Parent potato plants selected for crossing and the resulting potato populations

| | | Male Parent | | | | |
|---|---|---|---|---|---|---|
| | | | PED-PR-AB- | | PED-PR- | PED-PR-AD- |
| | | PED-PR-AB-rcs | sp | PED-PR-AB | AABB | sp |
| Female | PED-PR-AB-rso | | | | | BPP3 |
| Parent | PED-PR-CC-o | | BPP1 | SPP2 | | |
| | PED-PR-EF-rcs | BPP5 | | | | |
| | PED-PR-EF- | | BPP2 | | | |
| | rso-1 | | | | | |
| | PED-PR-EF- | BPP4 | | | | |
| | rso-2 | | | | | |
| | PED-PR-EF-o | | | | SPP1 | |

### PR4: Flow Cytometry

For each individual, young leaf tissue was used for flow cytometry. All parent MiMe plants described in section PR2 and used in the crosses described in section PR3 were shown to be diploid, having mean peak fluorescences consistent with diploid control plants. All individuals from BPP1, BPP2, BPP3, BPP4, and BPP5 were shown to be tetraploid with mean peak fluorescence values double that of diploid control plants, except for one triploid from BPP1 which was excluded from further analysis.

### PR5: Genotypic Evaluation

DNA samples were sequenced by Novogene. Libraries were prepared using the NEBNext® Ultra™ II for DNA Library Prep kit and paired-end 2×150 reads were sequenced on an Illumina NovaSeq 6000. Reads were aligned to the reference genome with BWA-MEM (v0.7.17-r1188) (Li, H. 2013) and alignments were sorted and indexed with samtools (v1.15.1). Markers were established and populations were genotyped as described in GM6.

For the parent non-MiMe plant PED-PR-CC-o and the MiMe plant PED-PR-AB-sp, DNA samples were sequenced to a depth of 25,923,949,200 bp, and 31,814,370,300 bp, respectively. For the BPP1 progeny plants, DNA samples were sequenced to an average depth of 51,874,541,384 bp. For the parent MiMe plant PED-PR-EF-rso-1, DNA sample was sequenced to a depth of 28,961,600,100 bp. For the BPP2 progeny plants, DNA samples were sequenced to an average depth of 52,349,190,474 bp. For the parent MiMe plants PED-PR-AB-rso and PED-PR-AD-sp, DNA samples were sequenced to a depth of 42,150,801,600 bp and 22,856,602,500 bp, respectively. For the BPP3 progeny plants, DNA samples were sequenced to an average depth of 52,905,004,980 bp.

For the parent MiMe plants PED-PR-AB-res, PED-PR-EF-res, and PED-PR-EF-rso-2, DNA samples were sequenced to a depth of 35,057,119,200 bp, 33,124,974,900 bp, and 38,116,453,200 bp respectively. For the BPP4 progeny plants, DNA samples were sequenced to an average depth of 49,270,594,500 bp. For the BPP5 progeny plants, DNA samples were sequenced to an average depth of 50,083,712,663 bp.

For the SPP1 progeny plants, DNA samples were sequenced to an average depth of 47,781,003,814 bp. For the SPP2 progeny plants, DNA samples were sequenced to an average depth of 28,116,787,950 bp.

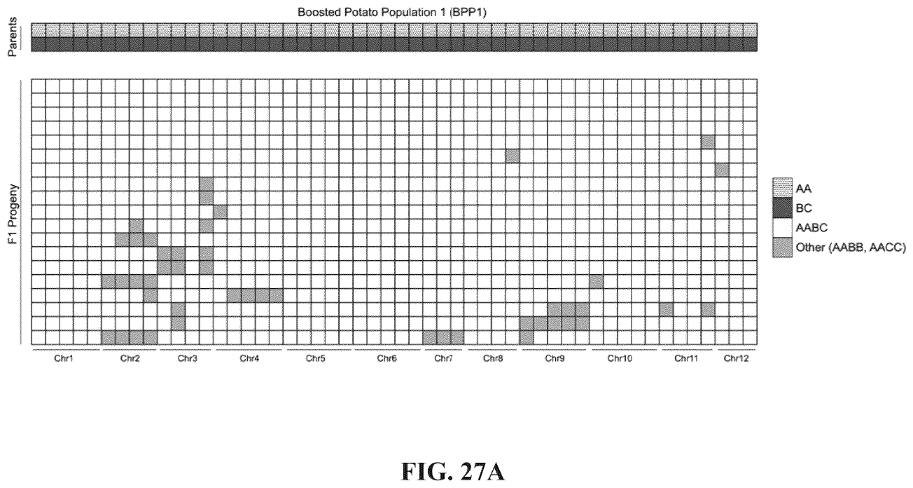

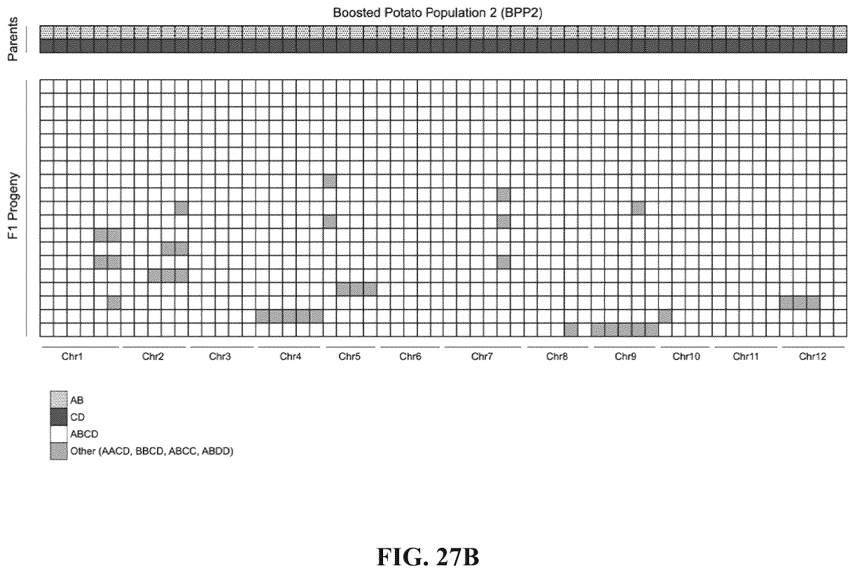

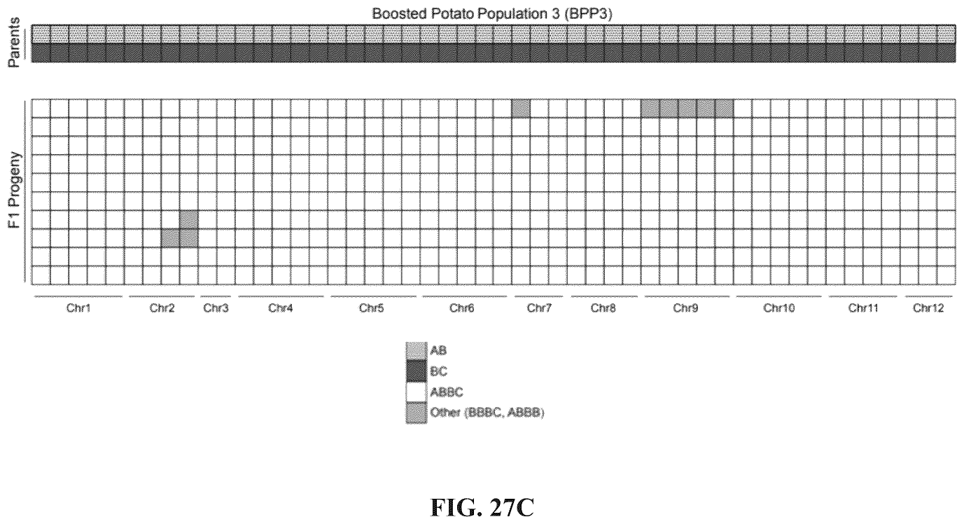

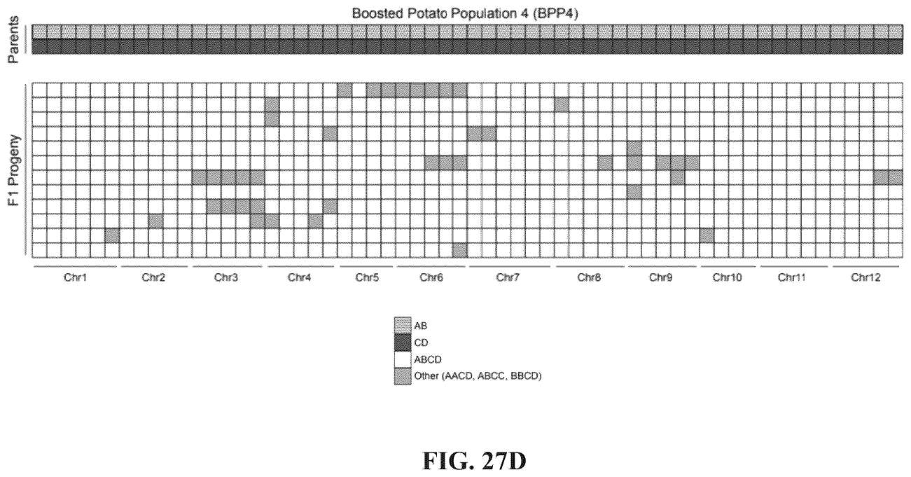

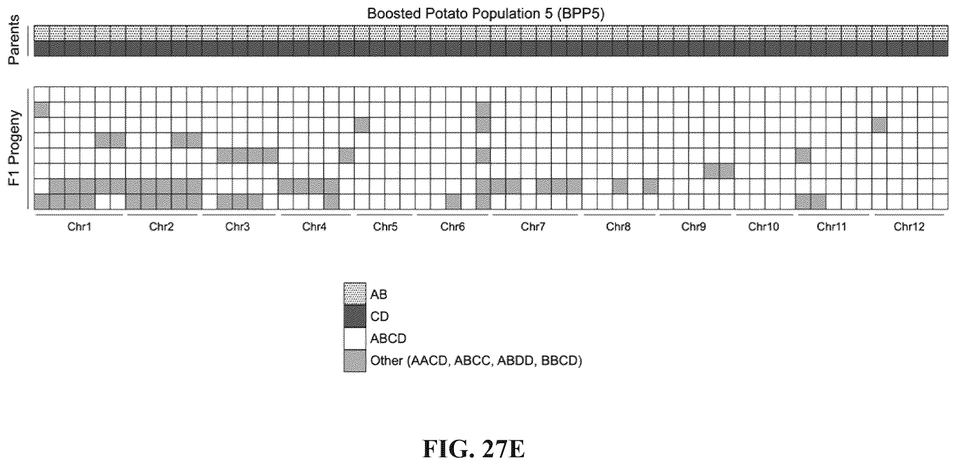

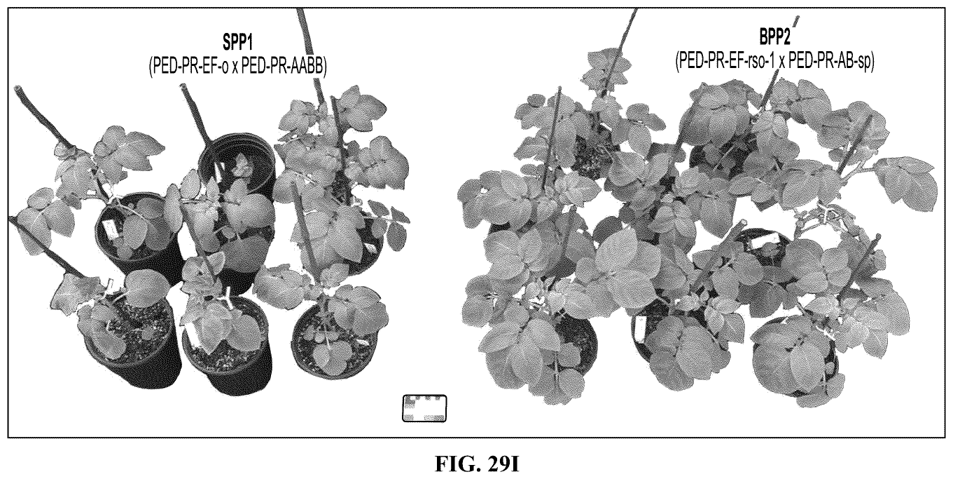

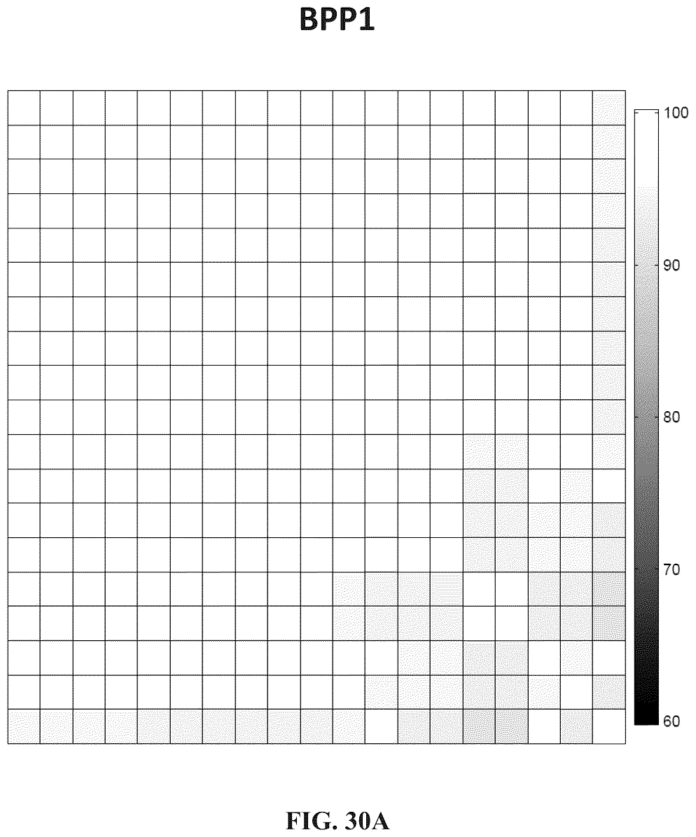

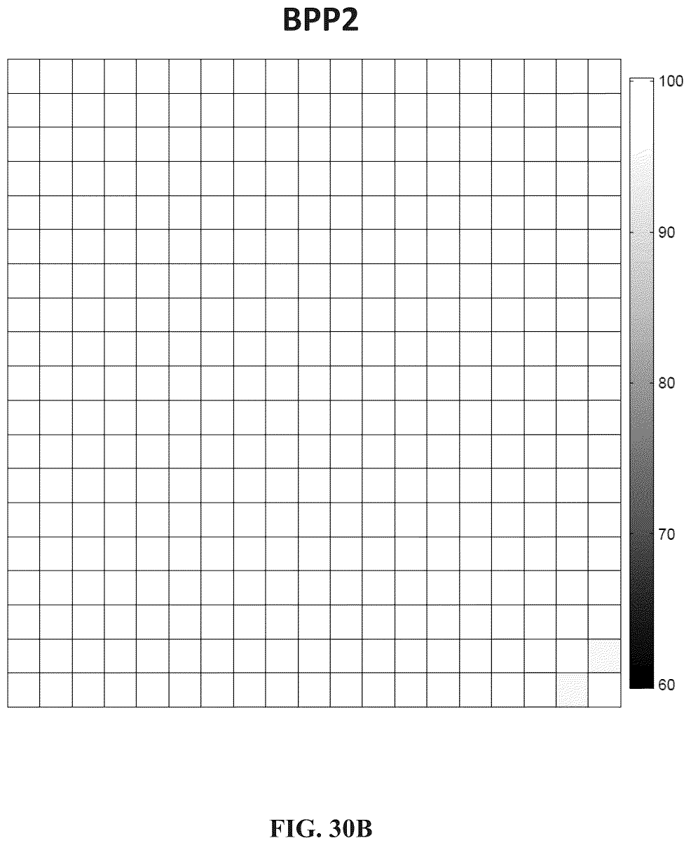

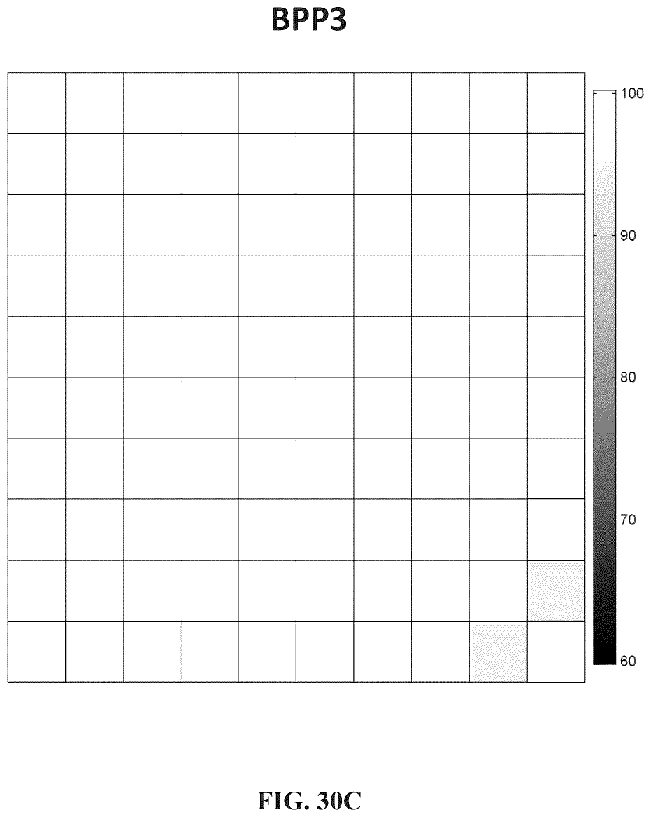

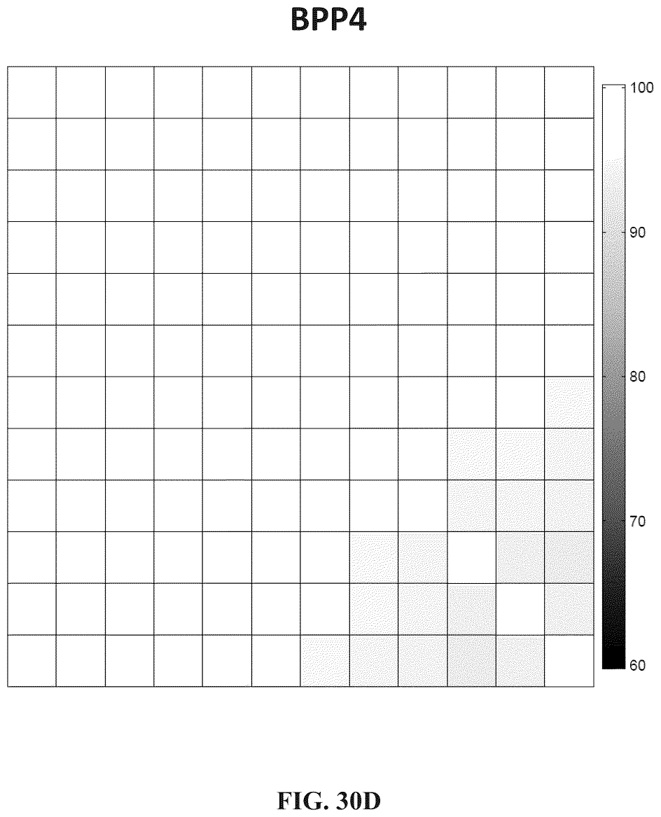

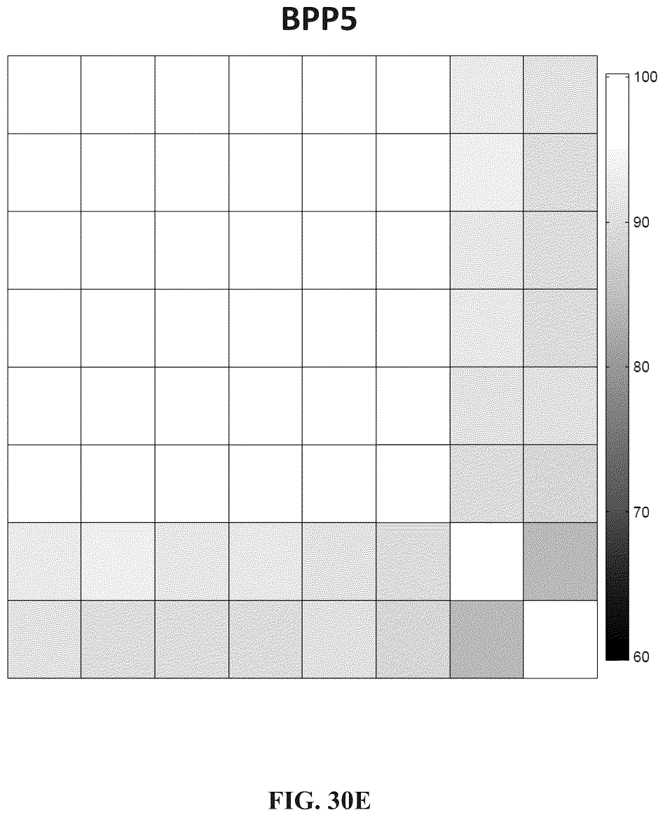

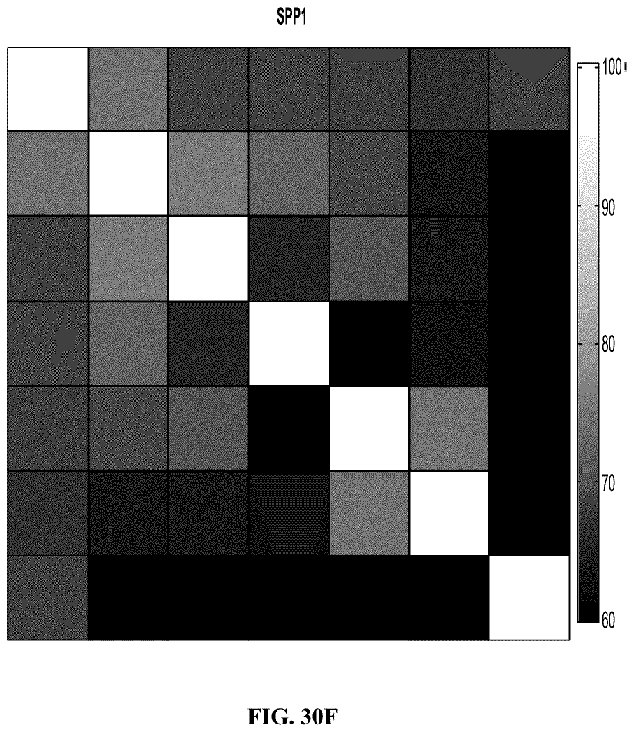

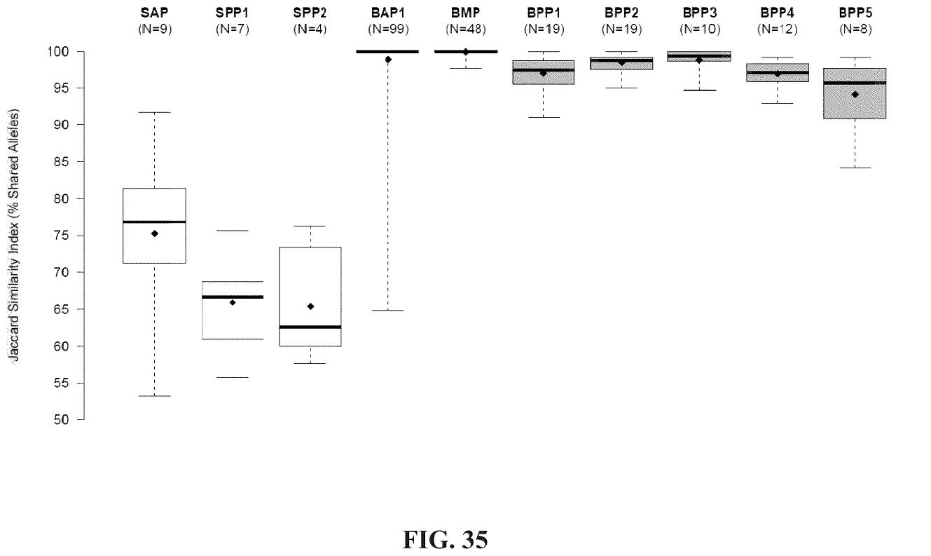

After filtering for high quality markers, a genotype matrix was assembled for BPP1 (FIG. 27A). Across the BPP1 population, the average pairwise genetic uniformity as measured by the Jaccard similarity coefficient was 97.0%. These data are illustrated by the population uniformity graph in FIG. 35 and the matrix in FIG. 30A.

After filtering for high quality markers, a genotype matrix was assembled for BPP2 (FIG. 27B). Across the BPP2 population, the average pairwise genetic uniformity as measured by the Jaccard similarity coefficient was 98.5%. These data are illustrated by the population uniformity graph in FIG. 35 and the matrix in FIG. 30B.

After filtering for high quality markers, a genotype matrix was assembled for BPP3 (FIG. 27C). Across the BPP3 population, the average pairwise genetic uniformity as measured by the Jaccard similarity coefficient was 98.8%. These data are illustrated by the population uniformity graph in FIG. 35 and the matrix in FIG. 30C.

After filtering for high quality markers, a genotype matrix was assembled for BPP4 (FIG. 27D). Across the BPP4 population, the average pairwise genetic uniformity as measured by the Jaccard similarity coefficient was 96.9%. These data are illustrated by the population uniformity graph in FIG. 35 and the matrix in FIG. 30D.

After filtering for high quality markers, a genotype matrix was assembled for BPP5 (FIG. 27E). Across the BPP5 population, the average pairwise genetic uniformity as measured by the Jaccard similarity coefficient was 94.2%. These data are illustrated by the population uniformity graph in FIG. 35 and the matrix in FIG. 30E.

Regarding the control populations, after filtering for high quality markers, the average pairwise genetic uniformity as measured by the Jaccard similarity coefficient for the SPP1 and SPP2 populations was 65.9% and 65.4%, respectively (uniformity by pairwise genetic matrix visualized in FIG. 30F and FIG. 30G, respectively). These data are illustrated by the population uniformity graph in FIG. 35.

### PR6a: Phenotypic Evaluation of BPP1

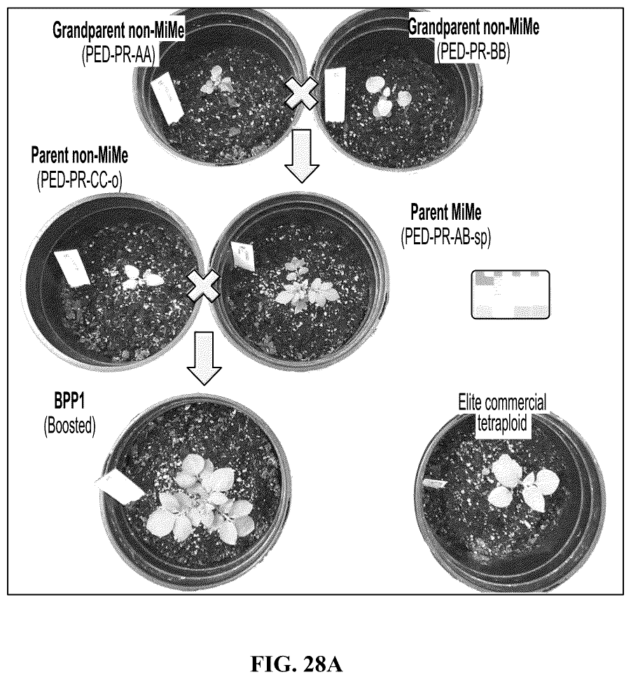

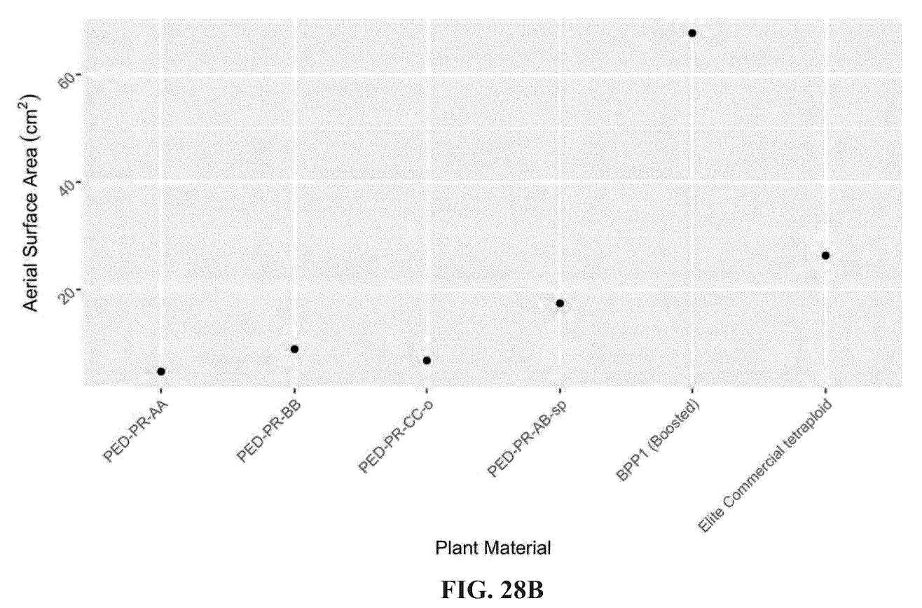

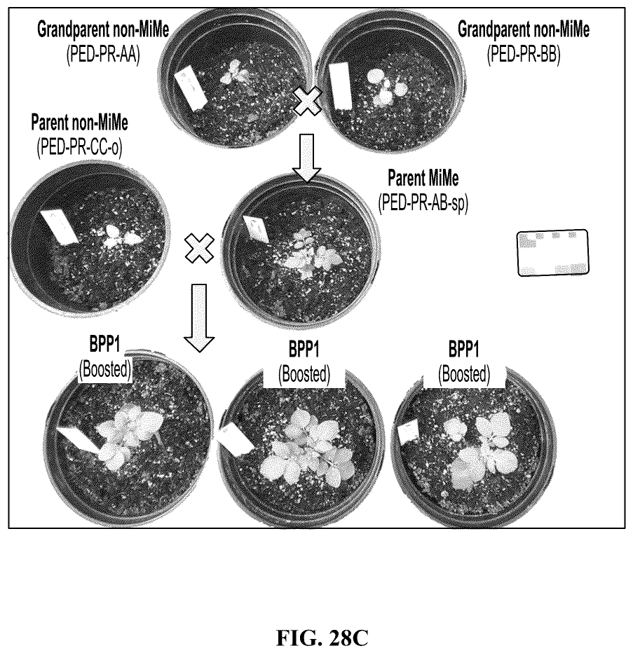

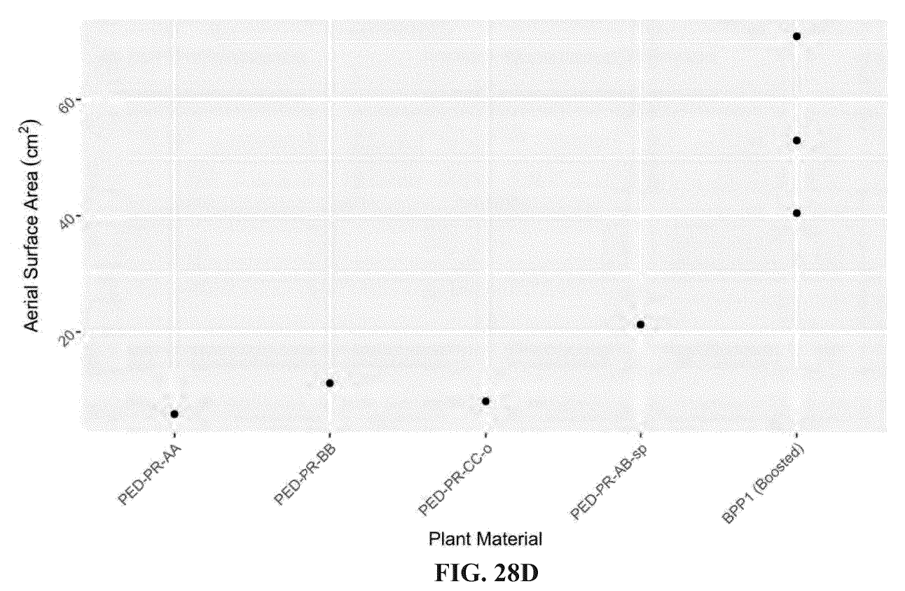

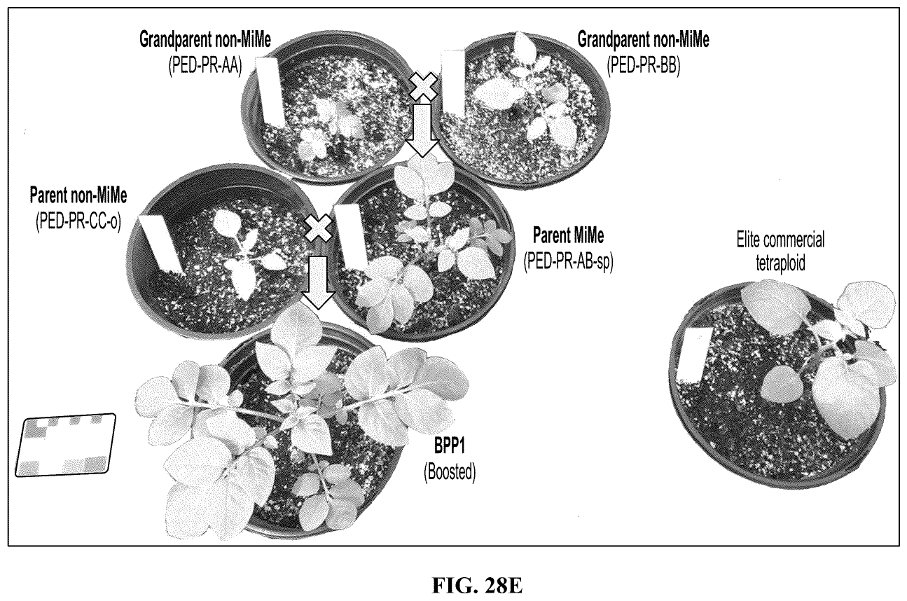

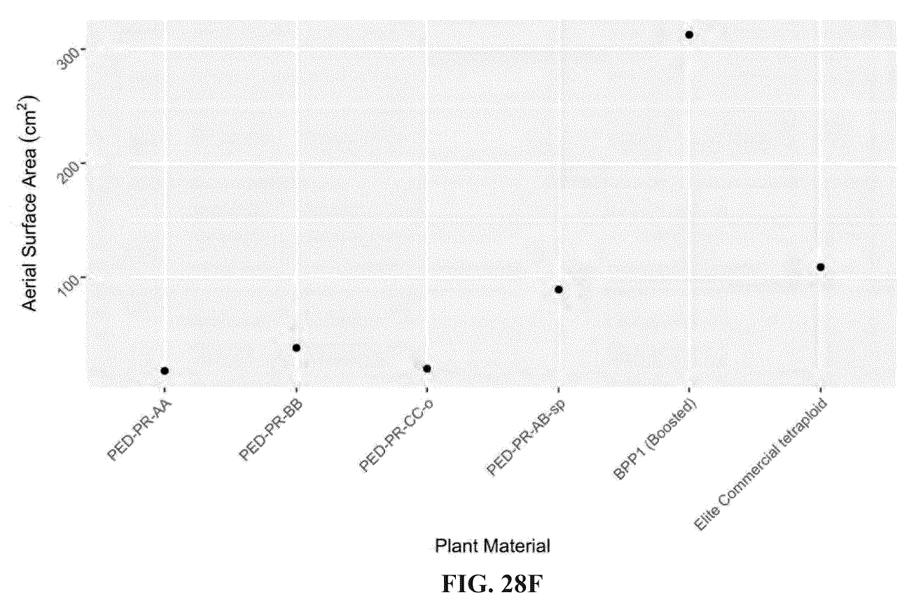

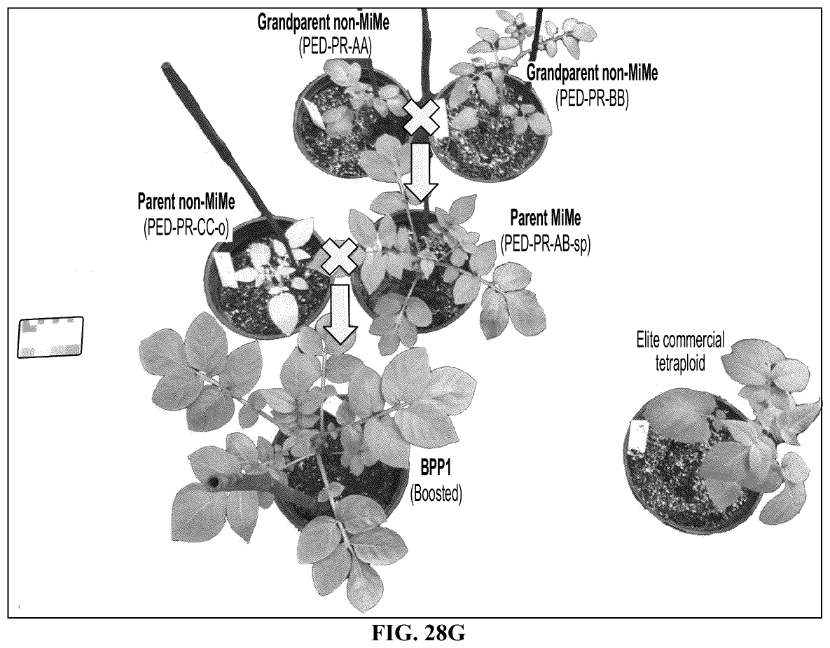

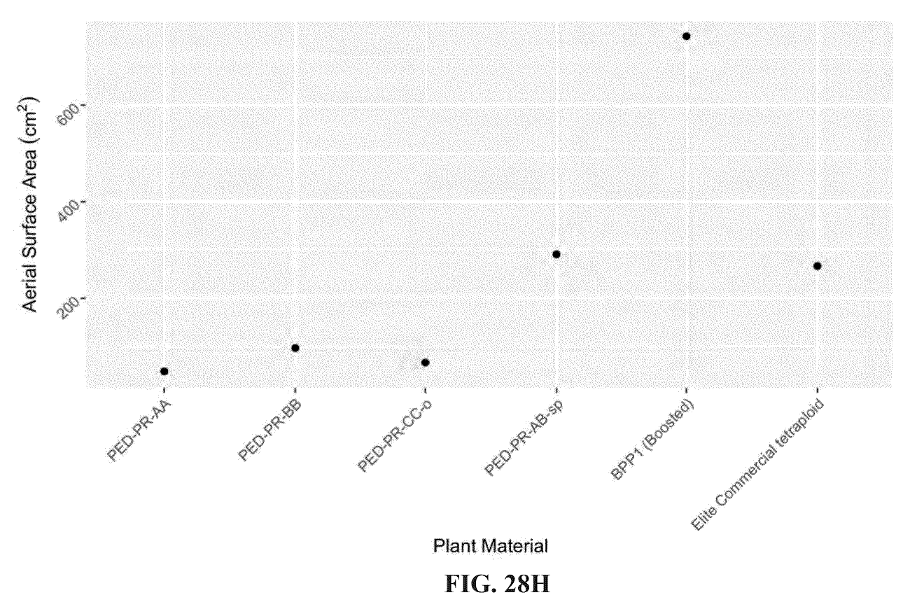

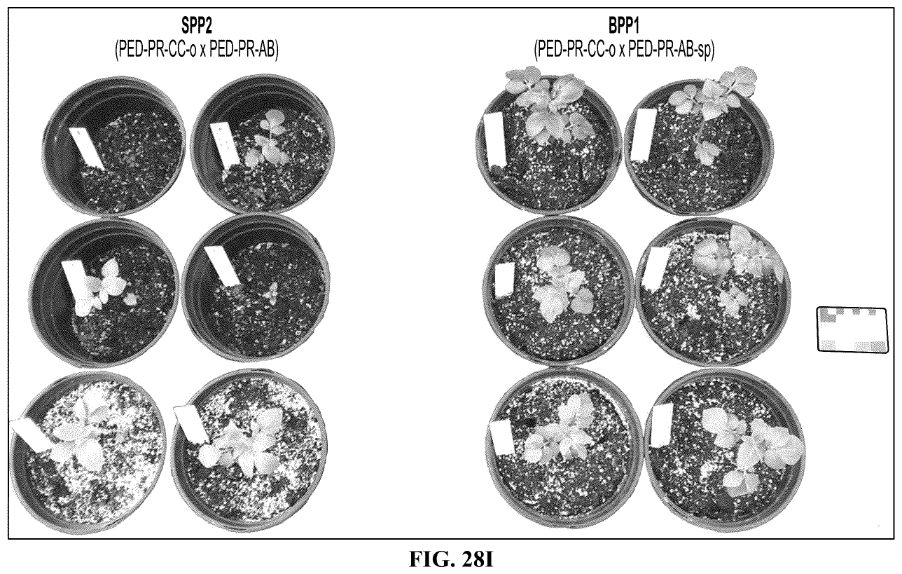

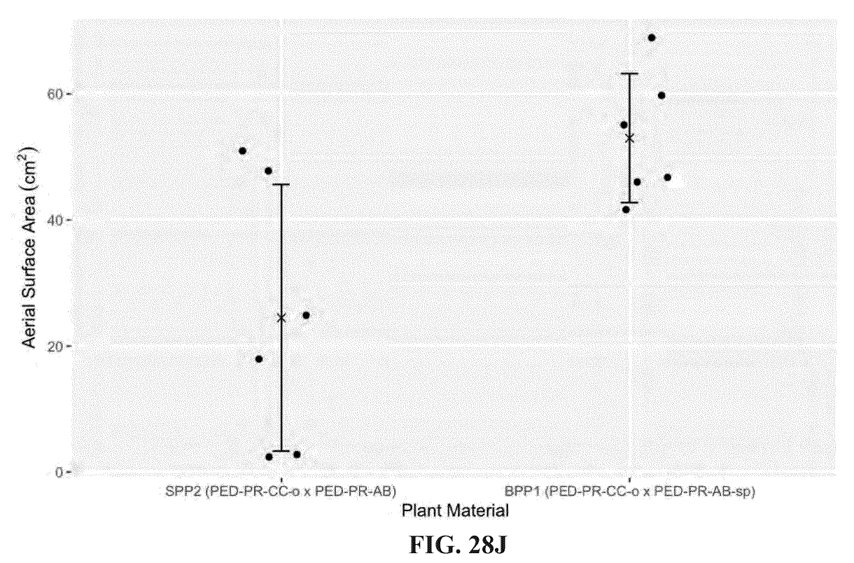

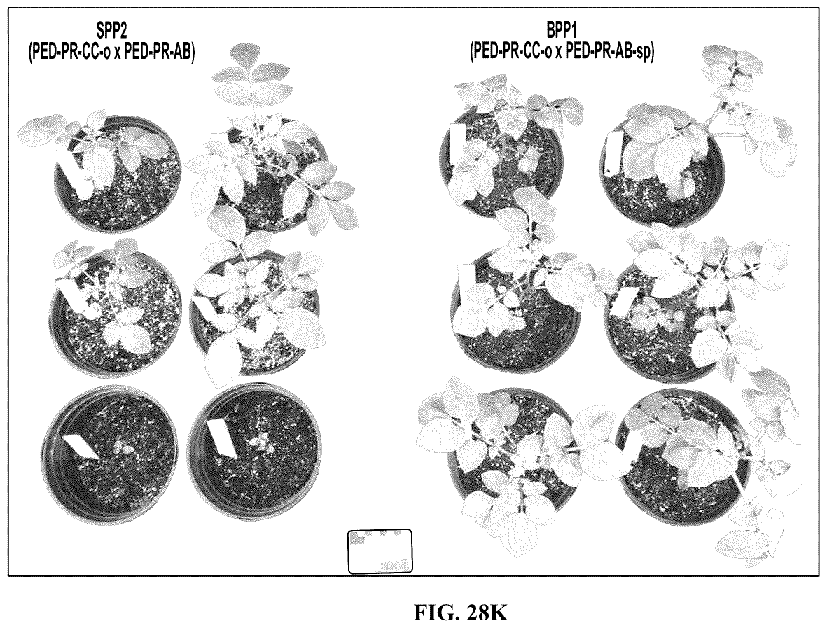

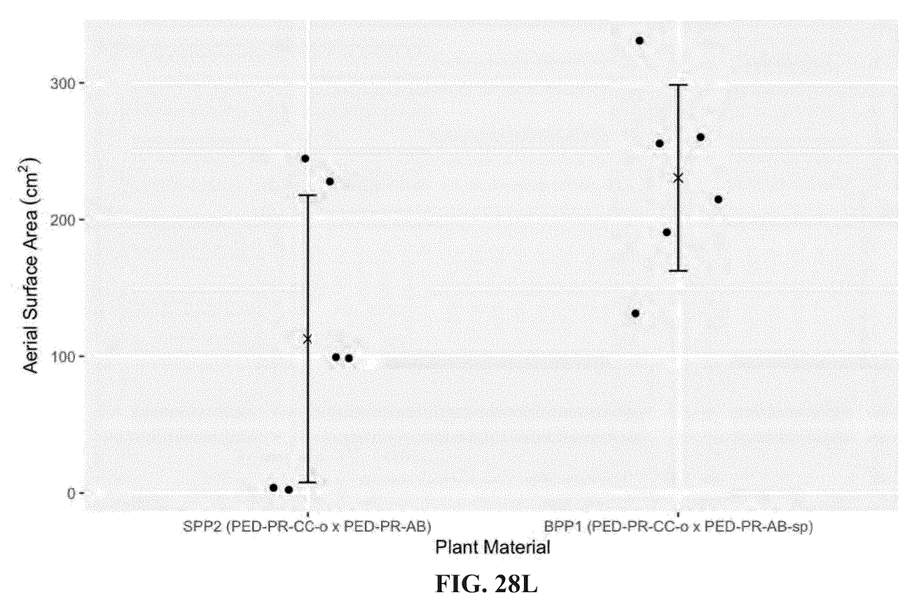

A potato plant from BPP1 is shown in FIG. 28A, FIG. 28E, and FIG. 28G adjacent to its grandparent plants, parent plants and an elite commercial tetraploid variety. For each figure, all plants are either 12 days post-planting (FIG. 28A), 19 days post-planting (FIG. 28E), or 26 days post-planting (FIG. 28G), respectively. For each figure, FIG. 28A, FIG. 28E, and FIG. 28G, respectively, (i) the plant on the top left of the photograph is the grandparent non-MiMe plant PED-PR-AA (doubled monoploid DM1-3 516 R44), (ii) the plant on the top right of the photograph, PED-PR-BB (selfed M6), is representative of the grandparent non-MiMe plant (M6), (iii) the plant on the middle left of the photograph is the parent non-MiMe plant PED-PR-CC-o, (iv) the plant on the middle right of the photograph is the parent MiMe plant PED-PR-AB-sp (spo11-1/ps), (v) the plant on the bottom left of the photograph is the BPP1 plant (labeled “BPP1 (Boosted)”), and (vi) the plant on the bottom right is an unrelated elite commercial tetraploid variety.

Six potato plants from BPP1 are shown in FIG. 28I and FIG. 28K compared to six plants resulting from SPP2. For each figure, all plants are either 16 days post-planting (FIG. 28I), or 23 days post-planting (FIG. 28K), respectively. The BPP1 plants demonstrated a phenotypically uniform size, growth, and appearance, whereas SPP2 plants were variable in size, growth, and appearance.

### PR6b: Phenotypic Evaluation of BPP2

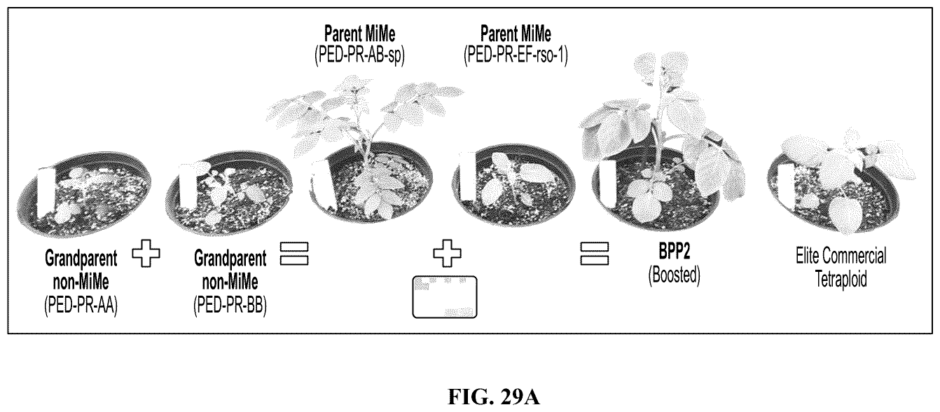

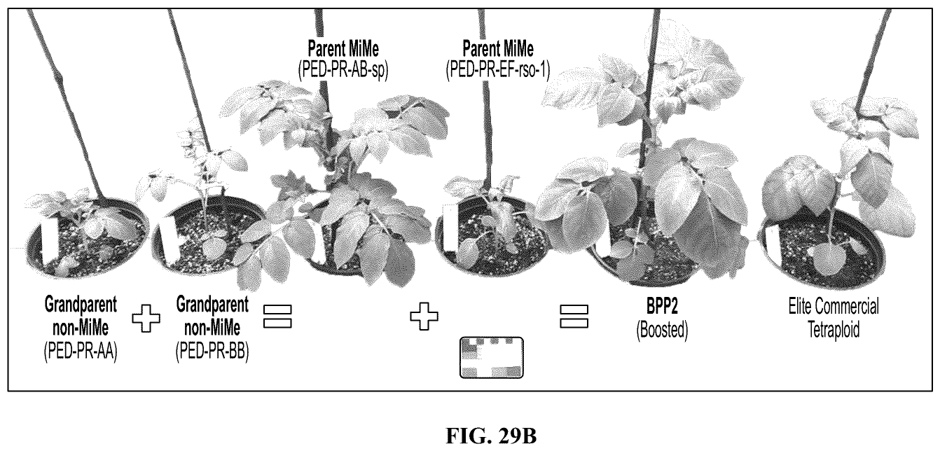

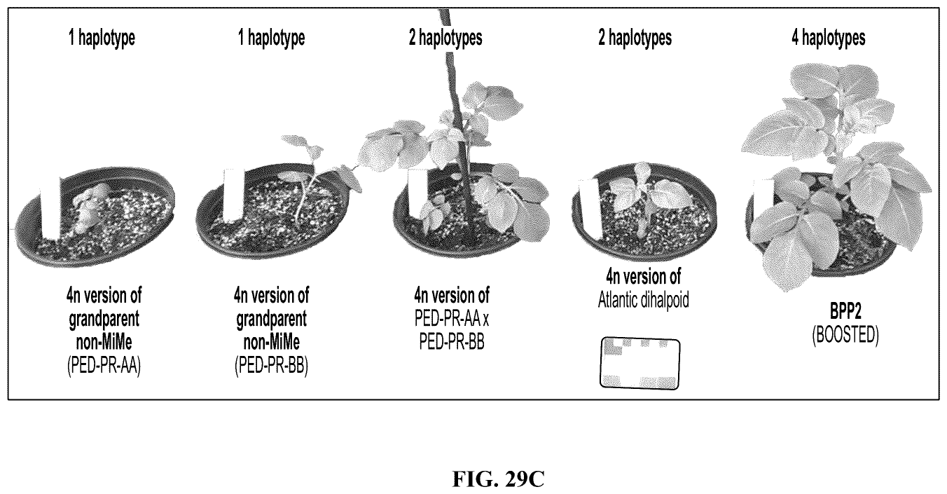

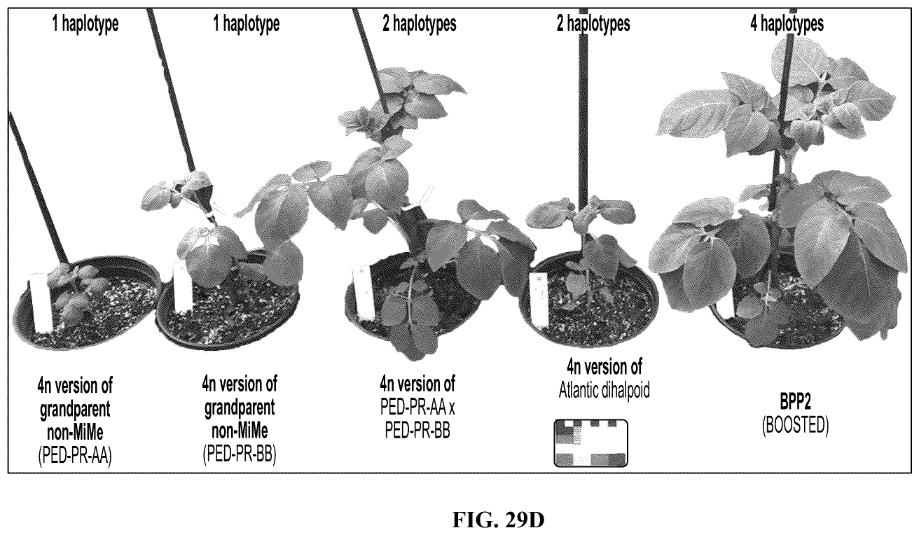

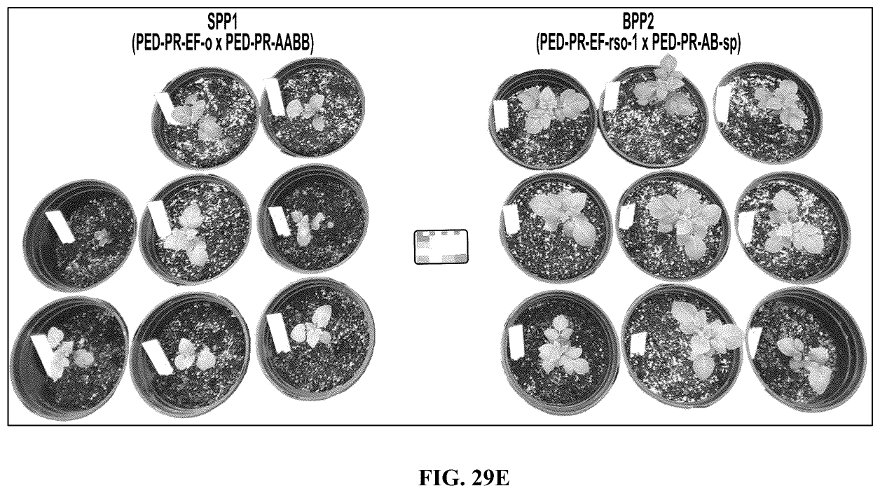

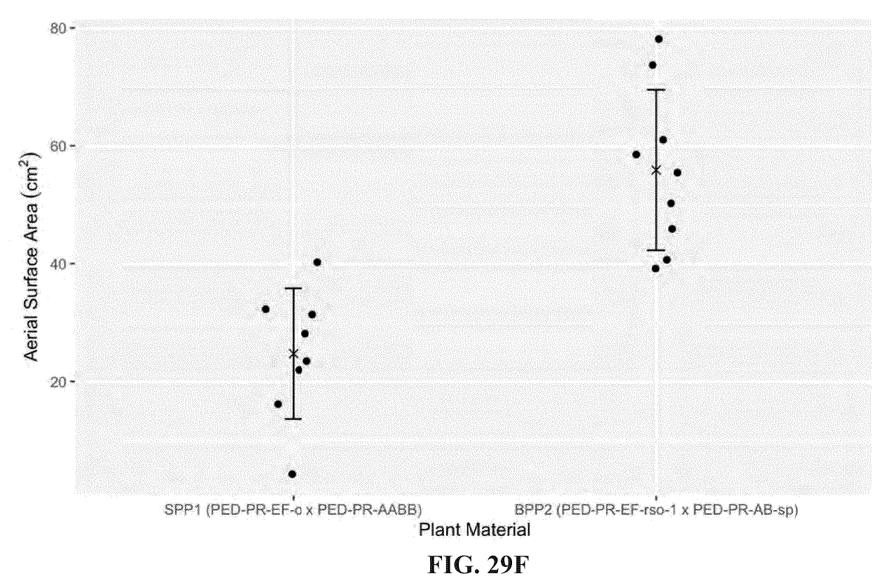

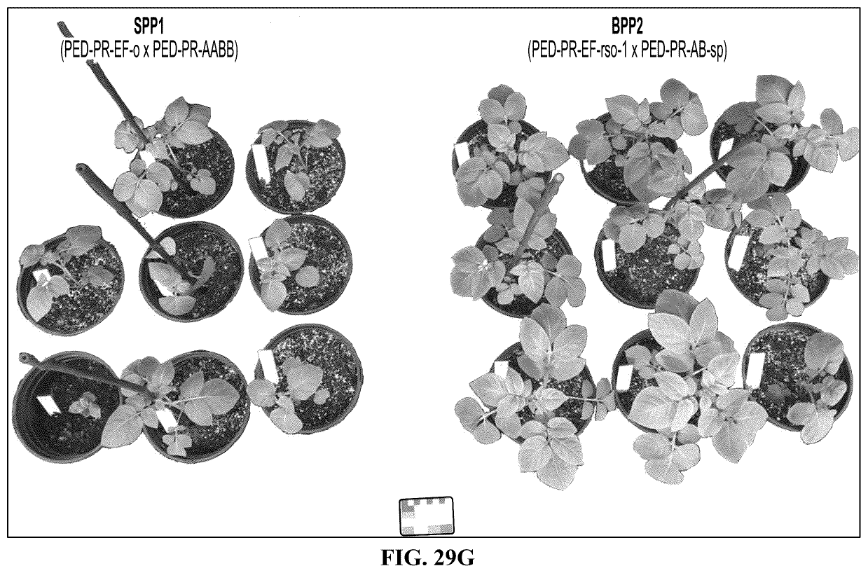

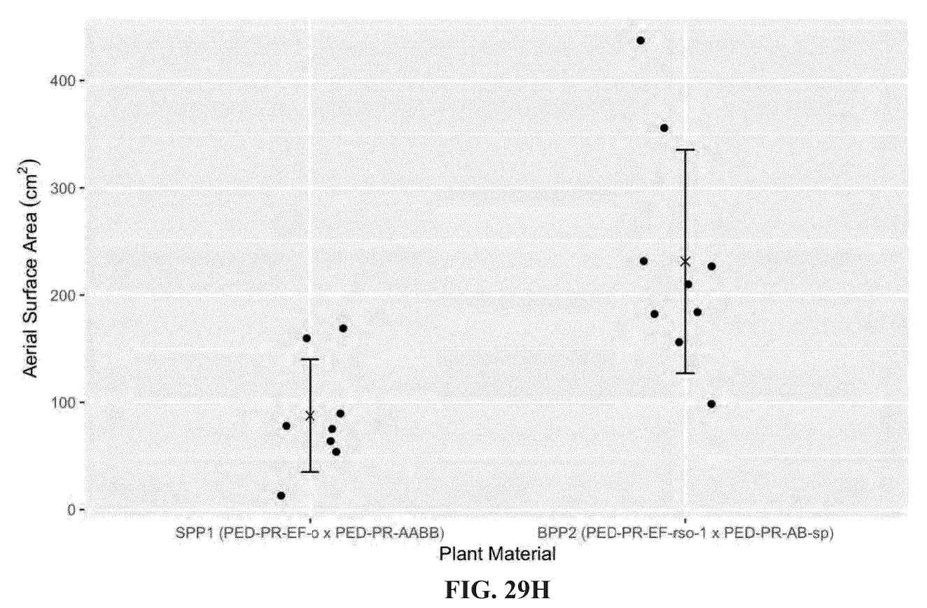

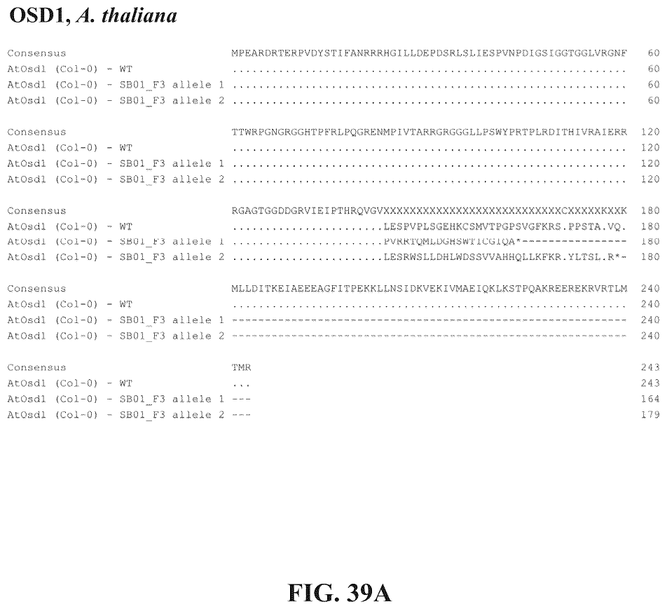

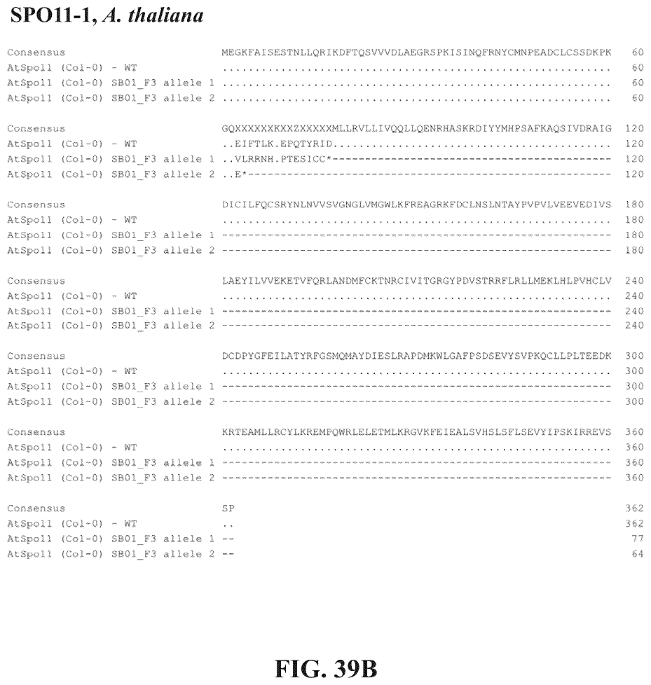

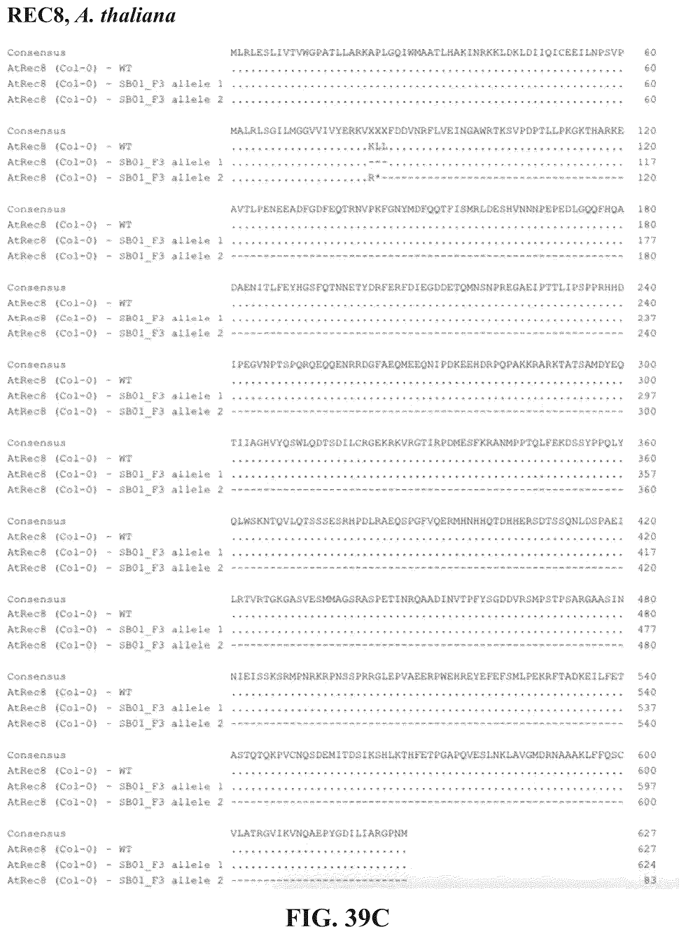

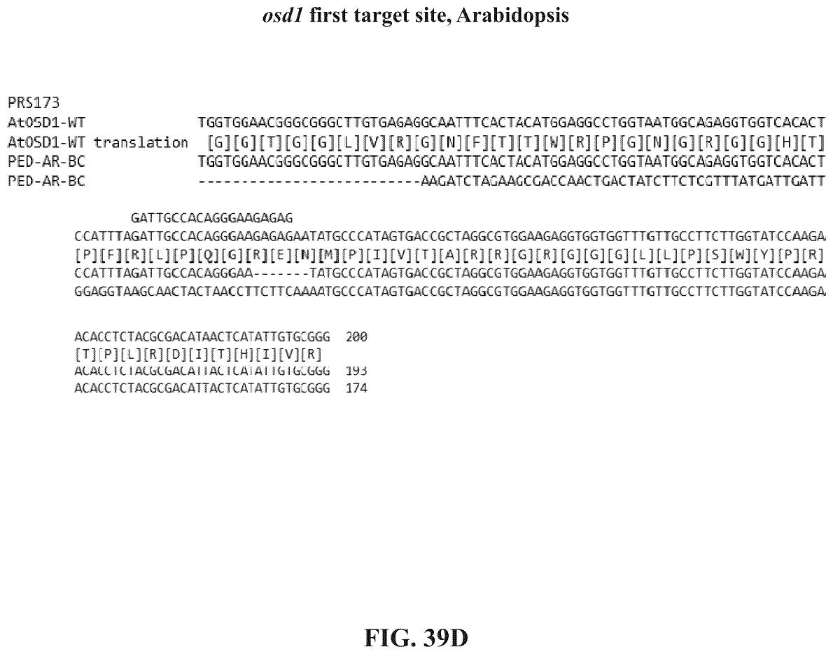

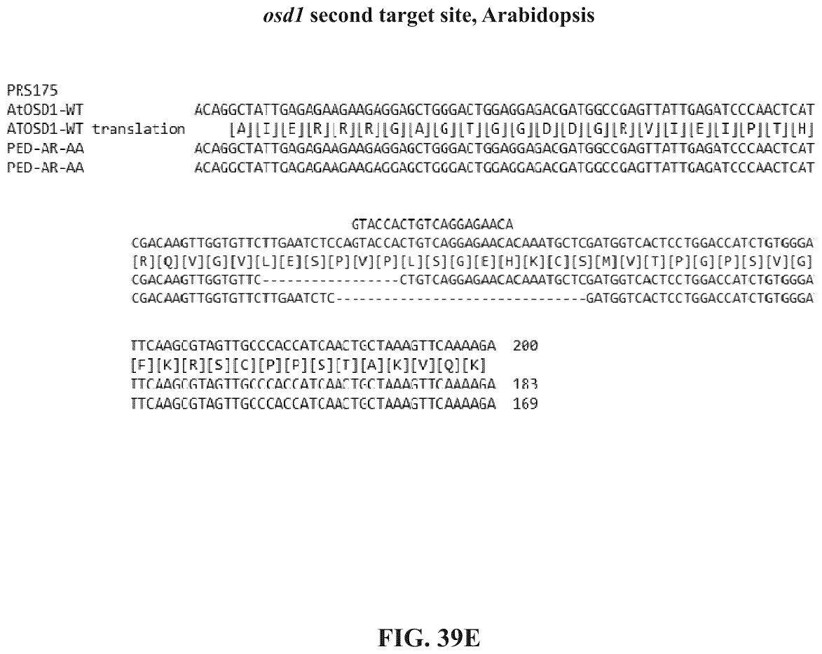

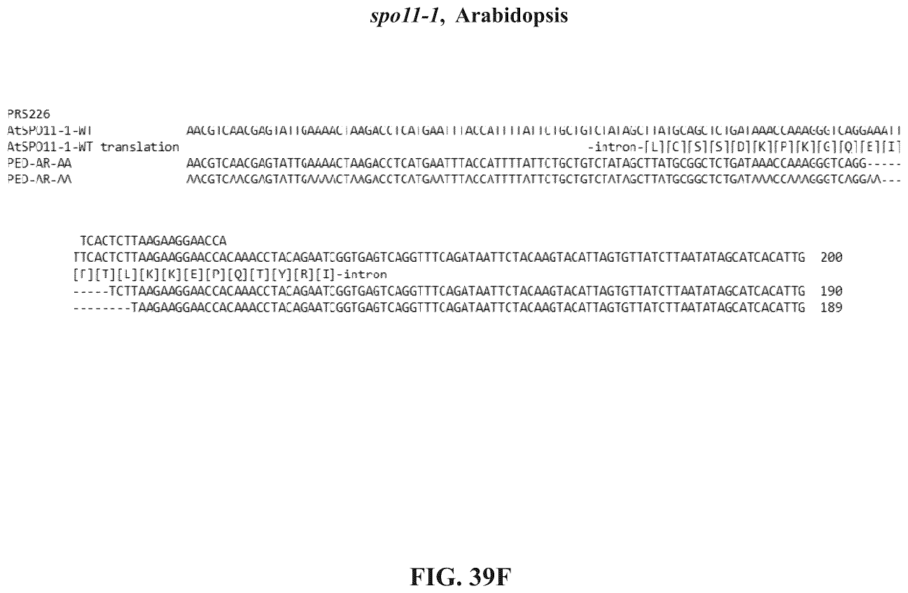

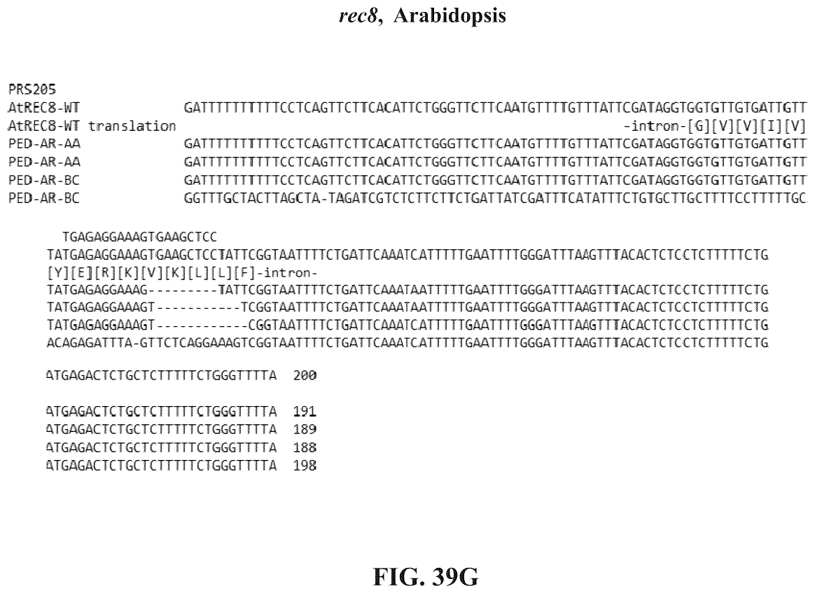

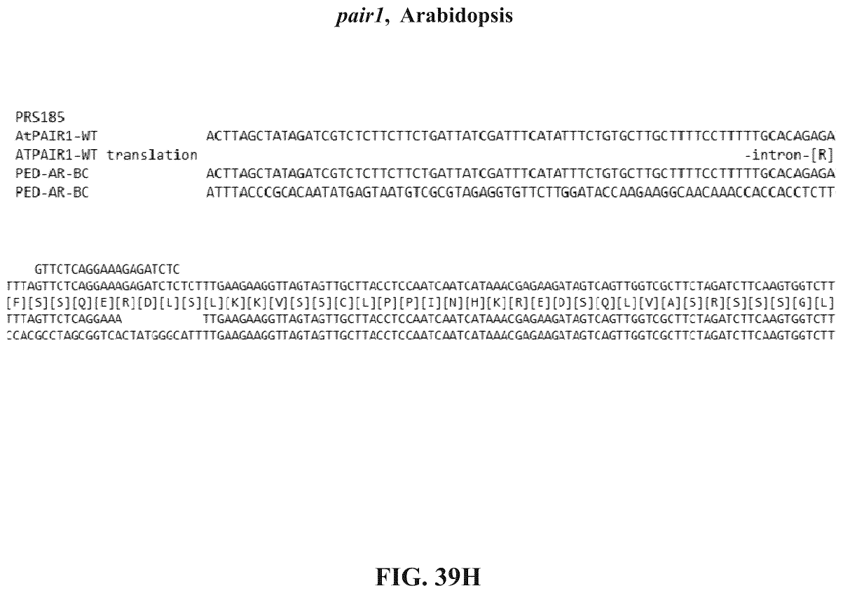

A potato plant from BPP2 is shown in FIG. 29A (23 days after planting) and FIG. 29B (29 days after planting) in the same photograph as its grandparent non-MiMe plants, parent MiMe plants and an elite commercial tetraploid variety. From left to right, the plants in FIG. 39A and FIG. 29B are (i) a grandparent non-MiMe plant (the doubled monoploid DM 1-3 516 R44), (ii) a selfed M6 representative of the grandparent non-MiMe plant (M6), (iii) a parent MiMe plant PED-PR-AB-sp (spo11-1/ps), (iv) a parent MiMe plant PED-PR-EF-rso-1 (rec8/spo11-1/os), (v) a BPP2 plant (labeled “BPP2 (Boosted)”), and (vi) an unrelated elite commercial tetraploid variety.

A potato plant from BPP2 is also shown in FIG. 29C (23 days after planting) and FIG. 29D (29 days after planting) in the same photograph as tetraploid versions of its grandparent non-MiMe plants and tetraploid versions of its parent MiMe plants. From left to right, the plants in FIG. 29C and FIG. 29D are (i) tetraploid (4n) version of the grandparent non-MiMe plant (the doubled monoploid DM 1-3 516 R44), (ii) tetraploid (4n) version of a twice selfed M6 representative of the grandparent non-MiMe plant (M6), (iii) tetraploid (4n) version of the DM1-3 516 R44 x M6 F1 hybrid (same parents as PED-PR-AB-sp which was also derived from a DM1-3 516 R44 x M6 F1 hybrid), (iv) PED-PR-EEFF, a tetraploid (4n) version of the Atlantic dihaploid from which PED-PR-EF-rso-1 was also derived), (v) a BPP2 plant (labeled “BPP2 (Boosted)”).

Nine potato plants from BPP2 are shown in FIG. 29E and FIG. 29G, and eight potato plants from BPP2 are shown in FIG. 29I, compared to eight plants from SPP1. For each figure, all plants are either 16 days post-planting (FIG. 29E), 23 days post-planting (FIG. 29G), or 29 days post-planting (FIG. 29I) respectively. The BPP2 plants demonstrated a phenotypically uniform size, growth and appearance, whereas the SPP1 plants were variable in size, growth and appearance.

### PC: Conclusions

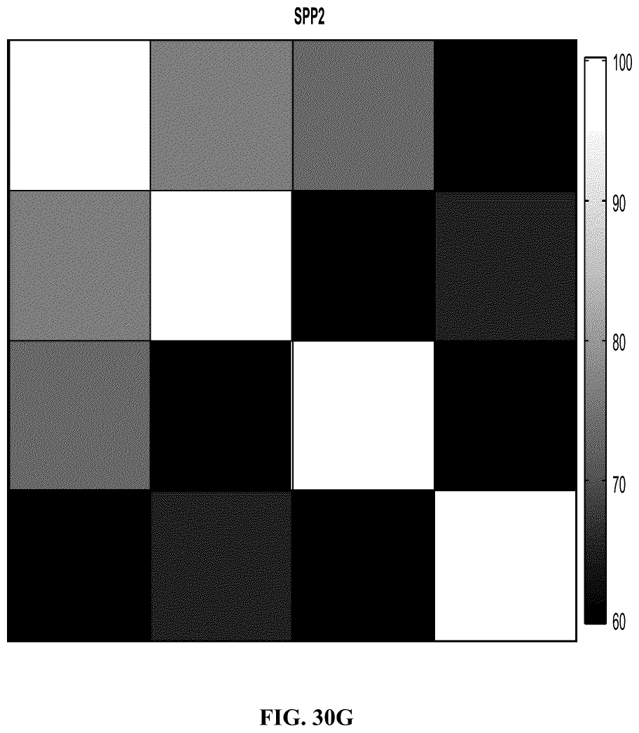

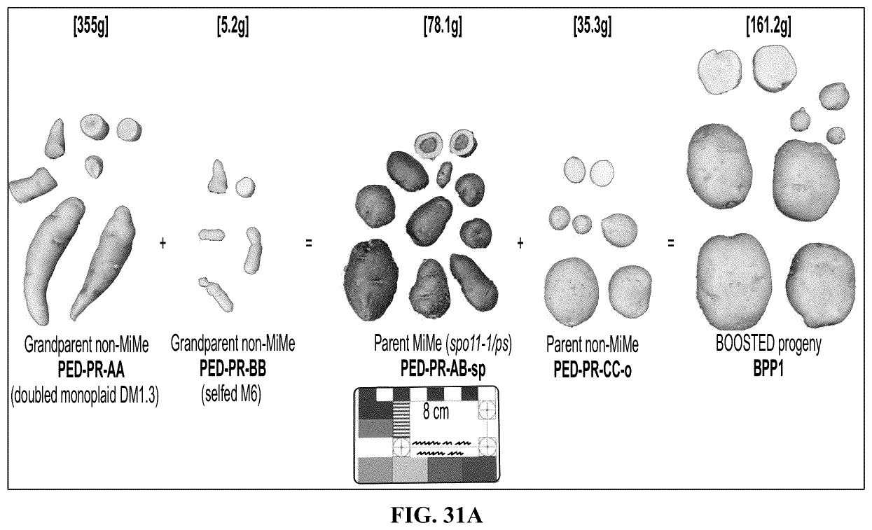

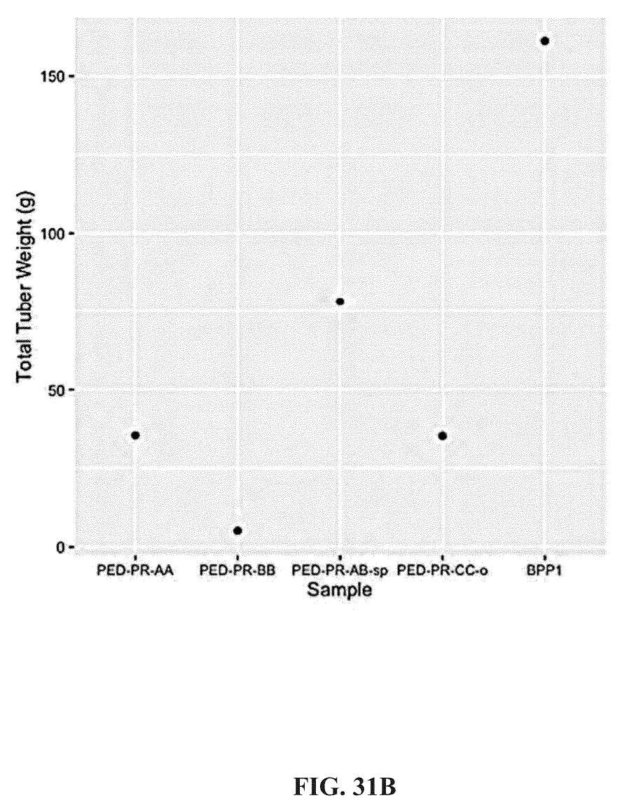

Alongside improved genetic and phenotypic uniformity, the BPP1 plants having three haplotypes exhibit many superior traits compared to both parent lines and grandparent plants. FIGS. 28A and 28C show that even by 12 days post-planting, relative to all compared plants, BPP1 plants have higher vigor, exhibit more dense foliage, and exhibit higher leaf surface area (quantified in FIGS. 28B and 28D). This trend intensifies through further days post-planting. By 19 and 26 days post-planting (FIG. 28E and FIG. 28G, respectively), the BPP1 plant has higher vigor, more nodes, more leaves, more leaf surface area (quantified in FIGS. 28F and 28H), and a more spreading growth habit than the compared plants. By 16 days post-planting, BPP1 plants exhibit these same superior traits when compared to the diploid SPP2 potato plants created from a non-MiMe cross of the wild-type version of same parents used in BPP1 (i.e. SPP2 is a cross between PED-PR-CC-o (selfed M18) and PED-PR-AB (an F1 hybrid of DM1-3xM6 without a MiMe genotype)) (FIG. 28I), and this continues at 23 days post-planting (FIG. 28K). The BPP1 plants' greater surface area per leaf is quantified in FIGS. 28J and 28L. The BPP1 plants also exhibited remarkable phenotypic uniformity, as compared to a cross of the wild-type version of the same parent plants that comprise the three haplotypes between them. At 16 days after planting, as shown in FIG. 28I, on the left, six representative progeny of SPP2, a cross between PED-PR-CC-o (selfed M18) and PED-PR-AB (an F1 hybrid of DM1-3xM6 without a MiMe genotype) exhibit very different phenotypes in terms of vigor, nodes, leaf number and leaf surface area. The phenotypic variability in SPP2 reflects the recombination between the two haplotypes present in the F1 hybrid and reduction leading to segregating recombined haploid gametes, “breaking” the two haplotypes of the heterozygous parent and preventing them from being passed to the progeny intact, and resulting in a lack of both genotypic (FIGS. 35 and 30G) and phenotypic (FIG. 28I) uniformity in SPP2. By contrast, on the right of FIG. 28I, six representative progeny of BPP1 (i.e., a cross between PED-PR-CC-o, selfed M18, and PED-PR-AB-sp, an F1 hybrid of DM1-3xM6 having a MiMe genotype) having three haplotypes shows both phenotypic uniformity and, on average, greater leaf surface area than the non-MiMe cross on the left, as quantified in FIG. 28J. Similar results are observed at 23 days after planting (FIGS. 28K and 28L). Finally, tubers harvested from BPP1 plants exhibit a remarkable increase in weight as compared to its parent and grandparent plants, showing the benefits of progressive heterosis of the three haplotypes of the BPP1 plants (FIGS. 31A-31B) and suggesting that potato plants comprising three or more haplotypes may have superior yield over diploid varieties and for one skilled in the art this shows the potential for superior yield and performance over cultivated tetraploids through successive generations of breeding. Furthermore, these results suggest that progressive heterosis can be maximized through the system described herein by combining the intact haplotypes of parents comprising superior diploid F1 hybrids with a MiMe genotype/phenotype with a third or fourth haplotype from either an inbred or a second superior diploid F1 hybrid with a MiMe genotype/phenotype to maximize variety performance as well as phenotypic and genotypic uniformity from true seed.

Similar to BPP1 plants, BPP2 plants having four haplotypes also exhibited superior traits compared to parent plants, grandparent plants, and elite commercial tetraploid plants. By 23 and 29 days post-planting, the BPP2 plants exhibit more vigor, taller height, and higher total leaf surface area than all compared plants (FIGS. 29A and 29B), even in crosses where the grandparent plants and parent plants are all tetraploid as well (FIGS. 29C and 29D). This shows that the improved performance is not simply due to an effect of ploidy (e.g. larger cells), but is also due to the composition of the plants having four haplotypes and improved progressive heterosis. Also, similar to BPP1 plants, representative BPP2 plants showed remarkably superior phenotypic uniformity, as compared to a cross of the wild-type version of the same parent plants that comprise the four haplotypes between them, as well as greater leaf surface area at both 16 (FIGS. 29E and 29F) and 23 (FIGS. 29G and 29H) days post-planting. At the time of filing the present disclosure, tuber data were not yet available for BPP2. However, given the superior tuber weight data observed for BPP1 having three haplotypes, those skilled in the art can readily expect that the combination of four haplotypes in BPP2 to exhibit superior tuber weight as well.

These experiments and associated figures demonstrate that despite the tested plants all being of the same age, grown in the same conditions, and produced from crosses using the same varieties of plant, the application of the MiMe genotype to generate a population of genetically-uniform polyploid seed having three or four haplotypes results in plants that display successful and superior progressive heterosis. These results show the surprisingly superior traits that can be achieved in plants grown from the populations of polyploid potato seed generated using the methods described herein. Provided that the source varieties for these experiments were chosen on a basis of availability rather than maximizing desired or specific phenotypes, those skilled in the art can readily appreciate that application of this disclosure to more precise breeding will yield even more superior stock.

### Example 3: Uniform Polyploid Blueberry (Vaccinium) Hybrids with Three or More Haplotypes

### VM: Methods

### VM1: Plant Materials and Protoplast Isolation

Diploid Vaccinium darrowii with pedigree ID PEDBB1 and a diploid Vaccinium darrowii with pedigree ID PEDBB2 are initiated in vitro using Woody Plant Medium (Lloyd, G., and B. McCown. (1980) Commercially-feasible micropropagation of mountain laurel, Kalmia latifolia, by use of shoot-tip culture. Proc. Int. Plant Prop. Soc. 30: 421-427.) with 4 mg/L Zeatin Riboside, 30 g/L sucrose, and pH of 5.2. In vitro plant material is grown under 16-hour day with cool white fluorescent lighting. Approximately one gram of leaves from 3-4-week-old explants are removed under aseptic conditions and stored in preconditioning medium for 1-24 hours before protoplast extraction. Other diploid or dihaploid Vaccinium darrowii, Vaccinium corymbosum and Vaccinium spp. are also selected and treated in the same manner.

Approximately thirty minutes before digestion leaves are sliced into thin sections approximately 1 mm in width and water is replaced with plasmolysis solution. After 30 minutes the plasmolysis solution is removed and replaced with cell and protoplast washing (CPW) (Reinert, J., and M. M. Yeoman. (1982) Plant cell and tissue culture. A laboratory manual. Plant cell and tissue culture. A laboratory manual.) solution containing 13% Mannitol, 10 g/L PVP-10, 10 g/L Cellulase RS (Yakult, Tokyo, 105-8664 JAPAN), 1 g/L MES (Sigma), 10 g/L Cellulase R-10 (Yakult, Tokyo, 105-8664 JAPAN), 10 g/L Hemicellulase and 1 g/L Pectolyase Y-23 (Gold Bio, St Louis MO) and having a pH of 5.2. The sliced leaves are placed in the digest solution and incubated overnight at 24° C. The next day protoplasts are liberated from the leaf tissue with 15-minute shaking at 40 RPM at room temperature.

Protoplasts are harvested through 100 μm sterile cell filters into sterile 50 mL conical tubes and are centrifuged at 100×g for 5 minutes. Supernatant is removed and replaced with 2 mL of wash solution containing CPW solution with 13% mannitol. Cells are gently resuspended by rocking and are slowly layered onto a 0.6 M sucrose solution. Tubes are centrifuged at 100×g for 15 minutes. After fifteen minutes a thick dark band of protoplasts appears at the interface of the two solutions. This band is harvested in one continuous motion using a sterile serological pipette and combined with 5 mL of transformation buffer containing 0.4 M mannitol, 15 mM MgCl2, and 4 mM MES with a pH of 5.2. Cells are quantified using a Bürker hemocytometer and stored at 4° C. in the dark until transfection. A sample is reserved to test cell viability using FDA staining as described by Larkin (1976. Purification and viability determinations of plant protoplasts. Planta. 128(3): 213-216).

### VM2: Transfection

Protoplasts are centrifuged at 50×g for 10 minutes and resuspended in a volume of transformation buffer to achieve a cell density of 1×10⁶ protoplasts/mL. Twenty microliters of freshly prepared RNP as described in section GM2 are added to the bottom of a 15 mL round bottom tube and 100 μL of protoplasts suspended in the transformation buffer are mixed with the RNP solution. An equal volume of the PEG solution (40% PEG4000, 100 mM CaCl₂, 0.2 M Mannitol) is gently mixed with cells and RNP by rotating the tubes. After a 5-15-minute incubation at room temperature the PEG solution is washed twice by centrifugation at 50×g for 10 minutes and application of a wash solution consisting of 0.4M D-Mannitol, 15 mM CaCl₂, and 5 mM HEPES. A final centrifugation is performed and transfected cells are resuspended into Woody Plant Medium supplemented with 9% Mannitol and 20 g/L glucose and are washed twice in the same medium.

### VM3: Encapsulation and Regeneration of Plants

An equal volume of transfected cells is combined with a 3.2% alginate solution and cells are gently mixed together. The resulting solution is pulled into a syringe and dispensed dropwise through a 23-gauge needle into 0.05M CaCl₂ solution containing 0.4 M D-Mannitol and 5 mM HEPES. Beads are incubated for 30 minutes at room temperature and are then washed with Woody Plant Medium supplemented with 9% Mannitol and 20 g/L glucose.

Plant hormone concentrations for regeneration are roughly based on Qiu et al (2018. Regeneration of Blueberry Cultivars through Indirect Shoot Organogenesis. HortScience. 53(7): 1045-1049) and the approach is briefly summarized as follows. After encapsulation the alginate beads are transferred to 24 well plate with 700 μL of Woody Plant Medium containing 20 g/L glucose, 500 mg/L Casein hydrolysate, 9% Mannitol with 2 mg/L Zeatin and 0.25 mg/L IBA. Plates are placed at 24° C. in the dark with continuous 40 RPM shaking. Beads remain in this medium for 3-4 weeks during which time cell division and signs of mini calli induction are observed through an inverted microscope and medium is exchanged. When mini calli reach 1 mm in size the light is gradually increased by first covering and then removing layers of cheesecloth under cool white fluorescent bulbs. After 3-6 weeks mini calli are liberated from alginate beads using a citric acid solution consisting of 20 mM Sodium Citrate and 0.5 M sorbitol. Mini calli are then washed in Woody plant medium and are plated on solid agar with Woody Plant Medium, 30 g/L sucrose, 2 mg/L Zeatin and 0.5 mg/L IBA with subculture every two weeks. Shoots emerge after 2-3 months of culture.

When shoots reach size of approximately 1 cm on, they are excised from the mini calli and are transferred to Woody Plant Medium containing 4 mg/L Zeatin Riboside and 30 g/L sucrose. Plants are placed under 16-hour day length under cool white fluorescent bulbs. Plants are multiplied with nodal segments and genotyped using the same sequencing methods described in Example 1's section GM3. Plants containing complete MiMe genotypes are further multiplied in vitro under the same conditions.

### VM4: Plant Growth and Crossing Conditions

Regenerated plants with confirmed edits in each target gene including REC8, OSD1, and PAIR1 and/or SPO11 are transplanted from tissue culture into peat substrate after making a fresh basal cut on each shoot and dipping the plant in rooting powder containing 0.1% IBA (Bonide, Oriskany NY, USA). Plants are acclimatized in a growth chamber at 20° C. with 60% relative humidity and 16-hour daylength. After two to three weeks plants are transplanted to 1-gallon pots and are grown under greenhouse conditions suitable first for vegetative growth and later for flower bud initiation.

When plants produce flowers, pollen is collected from the male and cross pollination is performed. The cross resulting from PEDBB1E and PEDBB2E is designated FAMBB1E-BB2E. Seeds are extracted with standard methods and are cleaned and dried. Dried and cleaned seeds are acid scarified and cold stratified according to routine methods for Vaccinium.

VM5: Genotyping of Uniform Polyploid Hybrids with Three or More Haplotypes

Following scarification approximately 200 cleaned seeds from FAMBB1E-BB2E are surface disinfested for 20 minutes in a sonic bath using a ten percent bleach solution containing 20 μL of Tween 20 (Sigma) in a final volume of 50 mL. Disinfested seeds are rinsed twice with sterile deionized water and are germinated in hormone free Woody Plant Medium with 30 g/L sucrose and 16-hour daylength under cool white fluorescent bulbs. Plants are transferred every two weeks to fresh medium and leaves are removed for DNA extraction during this process.

High quality genomic DNA is extracted from 100 randomly selected plants from FAMBB1E-BB2E using Qiagen DNeasy Plant Mini kits (QIAGEN, Germantown, MD, USA). Purity is determined using a spectrophotometer, Nanodrop™ One-C (Thermo Fisher), and DNA is quantified on a fluorometer, Qubit™ Flex Fluorometer (Q33327). Samples are adjusted to a final concentration of 20 ng/μL and are genotyped using Rapid Genomics Flex-Seq™ Services (Gainesville, Florida, USA). Following a standard genotyping pipeline for autopolyploids, the results are filtered for quality. Genotypic markers specific to the edited parent and indicative of unique haplotypes for PEDBB1E and PEDBB2E are identified and used to determine if the full complement of chromosomes was transmitted to each of the progeny.

### VR: Results

### VR1: Selection of Regenerated Plants

Regenerated plants are screened using the next-generation sequencing (NGS) methods described in Example 1's GM3, and approximately one percent of plants have mutations predicted to result in nonfunctional proteins of REC8, OSD1 and PAIR1 or in REC8, OSD1 and SPO11-1 or REC8, OSD1 and PAIR1 and SPO11-1. Out of between 2,000 and 10,000 regenerated plants approximately 20 to 100 unique plants are selected for evaluation in a partial diallel crossing scheme.

### VR2: Crossing and Seed Extraction

Hand pollinations are performed resulting in roughly 300 seeds per cross. Ten crosses are evaluated further and are germinated in vitro. The average germination rate per cross is between about 50% and about 95%. The cross of PEDBB1E and PEDBB2E with family designation of FAMBB1E-BB2E has a germination rate of between about 50% and about 95% and is selected for further evaluation with flow cytometry.

### VR3: Flow Cytometry

Both parents (PEDBB1E and PEDBB2E) are shown to be diploid having DNA content of approximately 1.2-2.1 pg per 2C nucleus relative to internal controls. Greater than 50% of the 100 progeny of PEDBB1E and PEDBB2E in FAMBB1E-BB2E are shown to be tetraploid with DNA content of approximately 2.4-4.2 pg per 2C nucleus and show similar variability between family members as observed in technical replicates of the same plant.

VR4: Genotypic Evaluation of Uniform Polyploid Hybrids with Three or More Haplotypes

Genotypic matrices and haplotyping results show specific loci with greater than three alleles in more than 70% of tetraploid members in FAMBB1E-BB2E and also show extended haplotype blocks with three or more haplotypes. After filtering for high quality markers the genotyping matrices for the 100 progeny of the tetraploid Family FAMBB1E-BB2E are greater than 70% similar.

### Example 4: Uniform Polyploid Blackberry (Rubus) Hybrids with Three or More Haplotypes

### RM: Methods

### RM1: Plant Materials and Protoplast Isolation

Diploid Rubus with pedigree ID PEDRR1 and a diploid Rubus with ID PEDRR2 are initiated in vitro using basal medium of Murashige and Skoog (1962. A revised medium for rapid growth and bioassays with tobacco tissue cultures. Physiologia plantarum 15(3): 473-497) with 2 mg/L 6-BAP and 30 g/L sucrose and grown under 16-hour daylength with cool white fluorescent lighting. Approximately one gram of leaves from 3-4-week-old explants are removed under aseptic conditions and stored in sterile water for 1-24 hours before protoplast extraction. Other dihaploid or diploid Rubus accessions are also selected and treated in the same manner.

Approximately thirty minutes before digestion the leaves are sliced into thin sections approximately 1 mm in width and water is replaced with plasmolysis solution. After 30 minutes the plasmolysis solution is removed and replaced with CPW solution (Reinert and Yeoman, 1982) containing 13% Mannitol (Sigma), 10 g/L PVP-10 (Sigma), 10 g/L Cellulase RS (Yakult, Tokyo, 105-8664 JAPAN), 10 g/L Hemicellulcase and 1 g/L Pectolyase Y-23 (Gold Bio, St Louis MO), 1 g/L MES (Sigma) and having a pH of 5.6. Sliced leaves in the digest solution are then incubated overnight at 24° C. The next day protoplasts are liberated from the leaf tissue with 15-minute shaking at 40 RPM at room temperature.

Protoplasts are harvested through 100 μm sterile cell filters into sterile 50 mL conical tubes and are centrifuged at 100×g for 5 minutes. Supernatant is removed and replaced with 2 mL of wash solution containing CPW solution with 13% Mannitol. Cells are gently resuspended by rocking and are slowly layered onto a 0.6 M Sucrose solution. Tubes are centrifuged at 100×g for 15 minutes. After fifteen minutes a thick dark band of protoplasts appears at the interface of the two solutions. This band is harvested in one continuous motion using a sterile serological pipette and combined with 5 mL of transformation buffer containing 0.4 M Mannitol, 15 mM MgCl2, 4 mM MES with a pH of 5.6. Cells are quantified using a Bürker hemocytometer and stored at 4° C. in the dark until transfection. A sample is reserved to test cell viability using FDA staining as described by Larkin (1976. Purification and viability determinations of plant protoplasts. Planta. 128(3): 213-216).

### RM2: Transfection

Protoplasts are centrifuged at 50×g for 10 minutes and resuspended in a volume of transformation buffer to achieve a cell density of 1×10⁶ protoplasts/mL. Twenty microliters of freshly prepared RNP as described in section GM2 are added to the bottom of a 15 mL round bottom tube and 100 μL of protoplasts suspended in the transformation buffer are mixed with the RNP solution. An equal volume of the PEG solution (40% PEG4000, 100 mM CaCl₂, 0.2 M Mannitol) is gently mixed with cells and RNP by rotating the tubes. After a 5-15-minute incubation at room temperature the PEG solution is washed twice by repeated cycles of centrifugation at 50×g for 10 minutes and resuspension in wash solution consisting of 0.4M D-Mannitol, 15 mM CaCl₂), and 5 mM HEPES. A final centrifugation is performed at the same speed and transfected cells are resuspended into Minimal Murashige and Skoog (MS) Medium (Linsmaier, E. M., and F. Skoog. 1965. Organic growth factor requirements of tobacco tissue cultures. Physiol. Plant. 18(1).) supplemented with 9% Mannitol and 20 g/L glucose.

### RM3: Encapsulation and Regeneration of Plants

An equal volume of transfected cells is combined with 3.2% alginate solution and cells are gently mixed together. The resulting solution is pulled into a syringe and dispensed dropwise through a 23-gauge needle into 0.05M CaCl₂ solution containing 0.4 M D-Mannitol and 5 mM HEPES. Beads are incubated for 30 minutes at room temperature and are then washed with Minimal MS Medium supplemented with 9% Mannitol.

Plant hormone concentrations for regeneration are roughly based on Huy et al. (1997. Protoplast isolation, culture, and cell differentiation in raspberry and blackberry cultivars (Rubus spp.). Angewandte-Botanik 71(3-4): 131-137) as well as Popescu and Isac (2000. High frequency shoot regeneration from leaf-derived callus in raspberry (Rubus idaeus L.) Eucarpia symposium on Fruit Breeding and Genetics 538, pp. 667-670) with modifications. The process is briefly summarized as follows. After encapsulation alginate beads are transferred to 24 well plate with 700 μL of Minimal MS Medium containing 20 g/L glucose, 500 mg/L Casein hydrolysate, 9% Mannitol, 10.74 μM NAA, 0.9 μM 2,4-D, and 1.74 μM BA. Plates are placed at 24 C in the dark with continuous 40 RPM shaking together with a feeder cell suspension of the same variety initiated as described by Huy et al (1997).

Beads remain in this medium with the feeder suspension for 4-6 weeks during which time cell division and signs of mini calli formation are observed through an inverted microscope. The medium and feeder suspension are refreshed every 2 weeks and when mini calli reach 1 mm in size the feeder suspension is removed and light is gradually increased by removing layers of cheesecloth under cool white fluorescent bulbs. After 3-6 weeks or when mini calli reach approximately 2 mm in diameter the mini calli are liberated from alginate beads using a citric acid solution consisting of 20 mM Sodium Citrate and 0.5 M Sorbitol. Mini calli are then washed in MS Medium and are plated on solid agar MS medium containing 30 g/L sucrose, 500 mg/L Casein hydrolysate, with 2.2 μM 2,4-D, and 13.32 μM BA with subculture every two weeks. Shoots emerge in 2-3 months.

When shoots reach size of approximately 1 cm, they are excised from the mini calli and are transferred to MS Medium (Murashige & Skoog, 1962) containing 0.1 mg/L IBA and 3 mg/L BA with 30 g/L sucrose. Plants are placed under 16-hour daylength under cool white fluorescent bulbs. Plants are multiplied from nodal segments and genotyped using the same sequencing methods described in section GM3. Plants containing complete MiMe genotypes are further multiplied in vitro under the same conditions.

### RM4: Plant Growth and Crossing Conditions

Regenerated plants with confirmed edits in each target gene including REC8, OSD1, and PAIR1 and/or SPO11 are transplanted from tissue culture into peat substrate after making a fresh basal cut on each shoot and dipping the plant in rooting hormone. Plants are acclimatized in a growth chamber at 20° C. with 60% relative humidity and 16-hour daylength. After two to three weeks plants are transplanted to 1-gallon pots and are grown under greenhouse conditions with a continued 16-hour daylength. When plants produce flowers pollen is collected from the male and cross pollination is performed. The cross resulting from PEDRR1E and PEDRR2E is designated FAMRR1E-RR2E. Seeds are extracted with standard methods and are cleaned and dried. Dried and cleaned seeds are acid scarified and cold stratified according to routine methods for Rubus.

RM5: Genotyping of Uniform Polyploid Hybrids with Three or More Haplotypes

Following scarification approximately 200 cleaned seeds are surface disinfested for 20 minutes in a sonic bath using a ten percent bleach solution containing 20 μL of Tween 20 (Sigma) in a final volume of 50 mL. Disinfested seeds are rinsed twice with sterile deionized water and are germinated in hormone free MS Medium with 30 g/L sucrose and 16-hour day length under cool white fluorescent bulbs. Plants are transferred every two weeks to fresh medium and leaves are removed for DNA extraction during this process.

High quality genomic DNA is extracted from 100 randomly selected plants from FAMRR1E-RR2E using Qiagen DNeasy Plant Mini kits (catalog number 69104) (QIAGEN, Germantown, MD, USA). Purity is determined using a spectrophotometer, Nanodrop™ One-C (ThermoFisher), and DNA is quantified on a fluorometer, Qubit™ Flex Fluorometer (Thermo Fisher). Samples are adjusted to a final concentration of 20 ng/μL and are genotyped using Rapid Genomics Flex-Seq™ Services (Gainesville, Florida, USA). Following a standard genotyping pipeline for autopolyploids the results are filtered for quality. Genotypic markers specific to the edited parent and indicative of unique haplotypes for PEDRR1-E and PEDRR2-E are identified and used to determine that the full complement of chromosomes was transmitted to each of the progeny.

### RR: Results

### RR1: Selection of Regenerated Plants

Regenerated plants are screened using the NGS sequencing methods described in section GM3 and approximately one percent of plants have mutations predicted to result in nonfunctional proteins of REC8, OSD1 and PAIR1 or in REC8, OSD1 and SPO11-1 or REC8, OSD1 and PAIR1 and SP011-1. Out of between 2,000 and 10,000 regenerated plants approximately 20 to 100 unique plants are selected for evaluation in a partial diallel crossing scheme.

### RR2: Crossing and Seed Extraction

Hand pollinations are performed resulting in roughly 200 seeds per cross. Ten crosses are evaluated further and are germinated in vitro. The average germination rate per cross is between about 50% and about 95%. The cross of PEDRR1E and PEDRR2E with family designation of FAMRR1E-RR2E has a germination rate of between about 50% and about 95% and is selected for further evaluation with flow cytometry.

### RR3: Flow Cytometry

Both parents (PEDRR1E and PEDRR2E) are shown to be diploid, having DNA content of approximately 0.49-0.8 pg per 2C nucleus relative to internal controls. Greater than 70% of the 100 progeny of PEDRR1E and PEDRR2E in FAMRR1E-RR2E are shown to be tetraploid with DNA content of approximately 1.16-1.6 pg per 2C nucleus and show similar variability between family members as observed in technical replicates of the same plant.

RR4: Genotypic Evaluation of Uniform Polyploid Hybrids with Three or More Haplotypes

Genotypic matrices and haplotyping results show specific loci with greater than three alleles in more than 70% of tetraploid members in FAMRR1E-RR2E and also show extended haplotype blocks with three or more haplotypes. After filtering for high quality markers, the genotyping matrices for the 100 progeny of the tetraploid Family FAMRR1E-RR2E are greater than 70% similar.

### Example 5: Generating Genetically Uniform Tetraploid Hybrid Maize

### ZM: Methods

ZM1: Plant Materials for crRNA Guide Screening

For crRNA guide screening, seeds of Zea mays B73, Hi-II, or Fast-Flowering Mini-Maize (“FFMM-AT6”) (McCaw et al. (2020) Development of a Transformable Fast-Flowering Mini-Maize as a Tool for Maize Gene Editing. Frontiers in Genome Editing. doi: 10.3389/fgeed.2020.622227) were planted in a plug tray filled with PRO-MIX HP, covered with a humidome, and underlain with a seedling heating mat that maintained a soil temperature of about 30° C. After 4 days, when seedlings germinated enough to reach light, they were covered by a box to block light. When the second leaf was about 10 cm longer than the first leaf, it was removed and used for protoplast isolation.

### ZM2: Protoplast Isolation

Approximately thirty minutes before digestion of the cell wall, 1-2 g of leaves were sliced into thin sections of about 0.5 mm in width and placed in a petri dish with 20 ml of digest solution (0.6M Mannitol, 10 mM MES (pH5.7), 1 mM CaCl₂, 5 mM $-mercaptoethanol, 0.1% Bovine Serum Albumin, 1.5% Cellulase RIO and 0.3% Macerozyme RIO). Leaf sections were then vacuum infiltrated for 30 minutes at room temperature, then incubated in the dark while shaking at 40 RPM for two hours. After two hours protoplasts were liberated by shaking at 80 RPM for 5 minutes. Protoplasts were then filtered through a 75 μM mesh and transferred to two round bottom tubes. The protoplasts were pelleted at 100×g for 5 minutes, then were washed twice by resuspending in a cool wash solution (0.6M mannitol, 20 mMKCl, 4 mM MES (pH5.7)) and pelleting. Finally, protoplasts were resuspended in 10 ml of wash solution and incubated on ice for one hour. Cells were quantified using a Burker hemocytometer and stored on ice in the dark until transfection. A sample was also reserved to test cell viability using FDA staining as described by Larkin (1976. Purification and viability determinations of plant protoplasts. Planta 128(3): 213-216).

### ZM3: Transfection

Immediately before transfection, a fresh polyethylene glycol (PEG) solution was prepared according to Cao et al. (2014. PEG-mediated transient gene expression and silencing system in maize mesophyll protoplasts: a valuable tool for signal transduction study in maize. Acta Physiologiae Plantarum. 36(5): 1271-1281.). Protoplasts were centrifuged at 100×g for 5 minutes and resuspended in a volume of MMG transformation buffer (4 mM MES, pH 5.7, 0.4 M mannitol and 15 mM MgCl₂) as described by Cao et al. (2014) to achieve a cell density of 1-2×10⁶ protoplasts/mL. 15 μL of RNP was freshly prepared as described in Example 1's section GM2 and added to wells of a 48-well plate. Alternatively, 20 μg of plasmid DNA total in 15 μL of sterile deionized water was added to a 48-well plate. Next, 200 μL of protoplast suspension was mixed with the RNP solution (or plasmid DNA) by pipetting with wide bore tips, after which the approximately 215 μL of PEG solution was added and gently mixed by pipetting up and down with a wide bore tip until Schlieren lines were no longer visible. After a 20-minute incubation at room temperature, 800 μl of incubation solution (0.6 M mannitol, 4 mM KCl, 4 mM MES) was added to the approximately 215 μL of PEG solution and mixed again by pipetting. The protoplasts were then pelleted from the resulting solution at 100×g for 2 minutes, after which the plates were rotated and centrifuged again at 100×g for 2 minutes. The supernatant was then removed, and 2 mL of incubation solution (0.6 M mannitol, 4 mM KCl, 4 mM MES) was added to the wells. Protoplasts were resuspended according to Cao et al. 2014 and then incubated in the dark for 2 days at 27° C. After this incubation, 30 ul of cells were collected from the bottom of the wells and pipetted into a PCR plate where they were lysed, and DNA was extracted using the Platinum Direct Polymerase kit (#A44647100 ThermoFisher Scientific). A sample from each well was also evaluated for cell viability using FDA staining as described by Larkin (1976. Purification and viability determinations of plant protoplasts. Planta 128(3): 213-216).

### ZM4: Plasmids for Biolistic Transformation

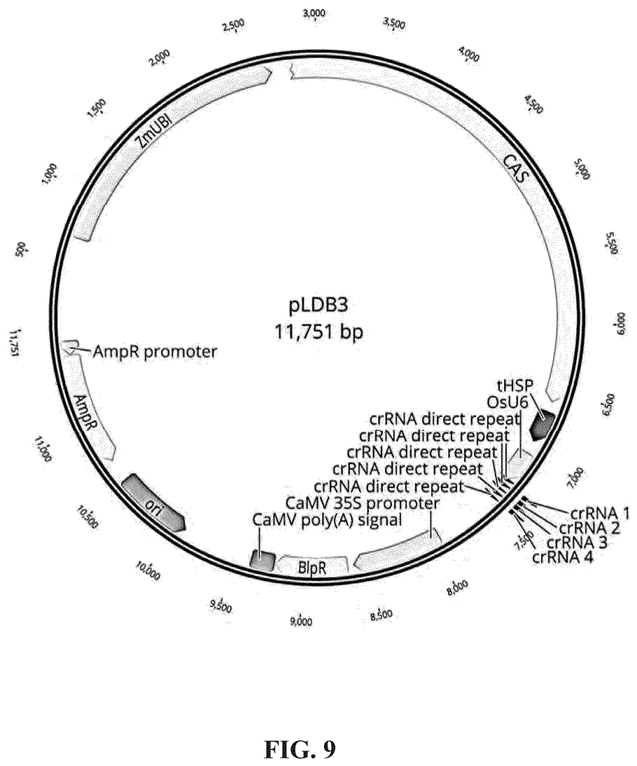

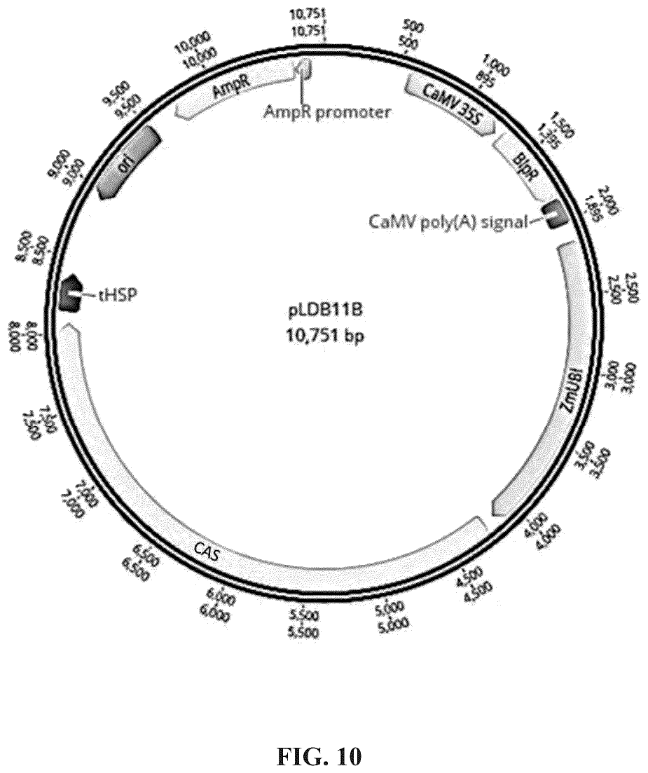

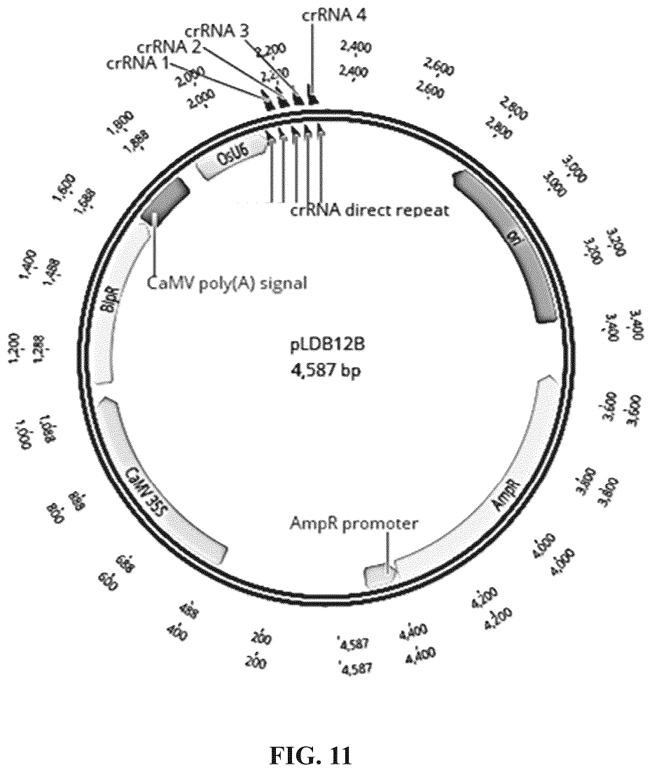

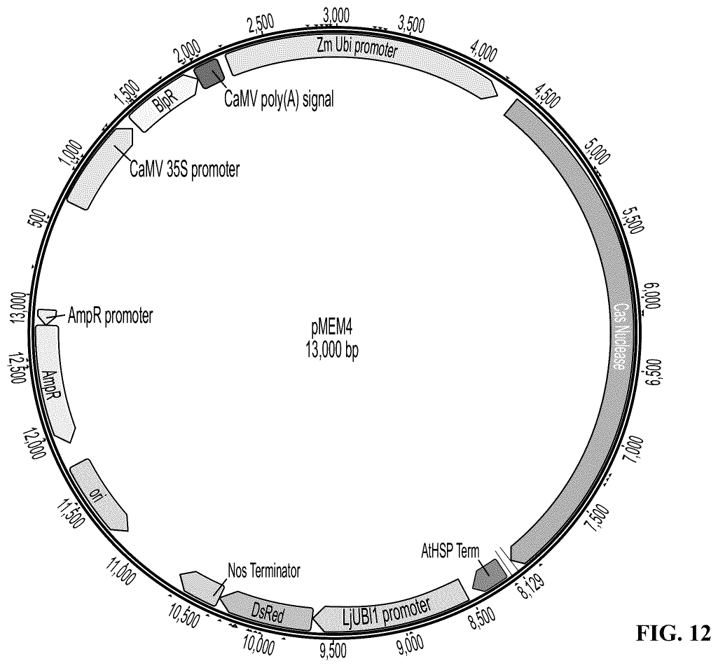

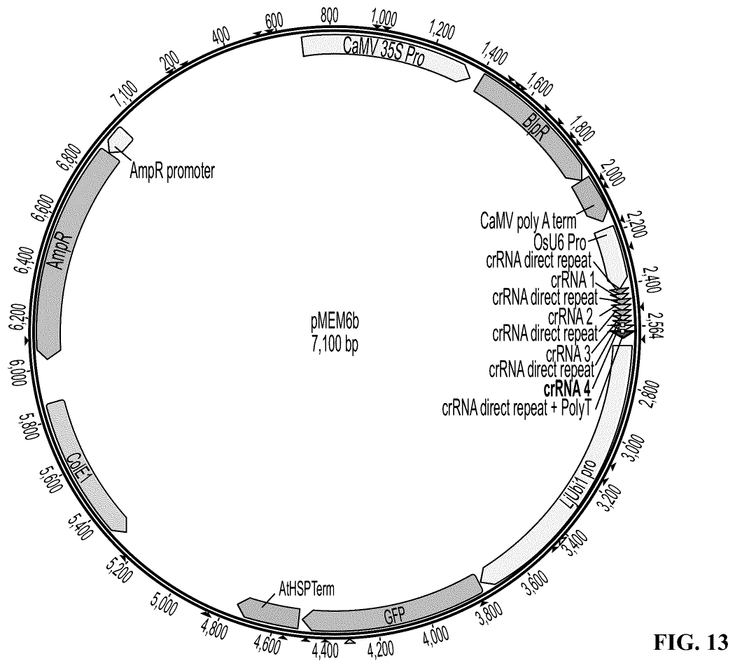

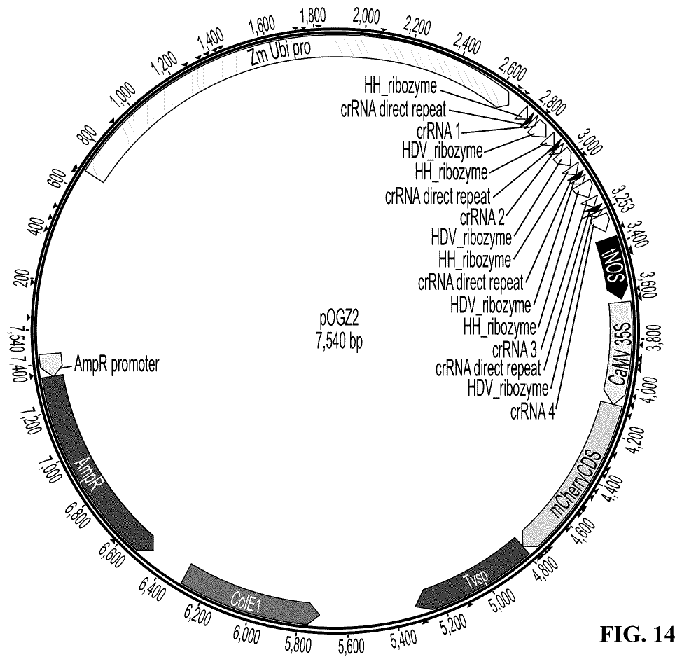

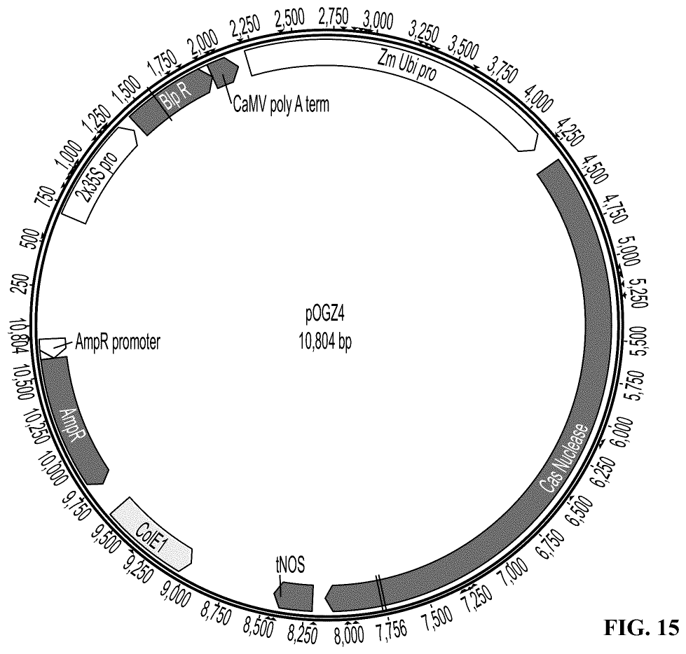

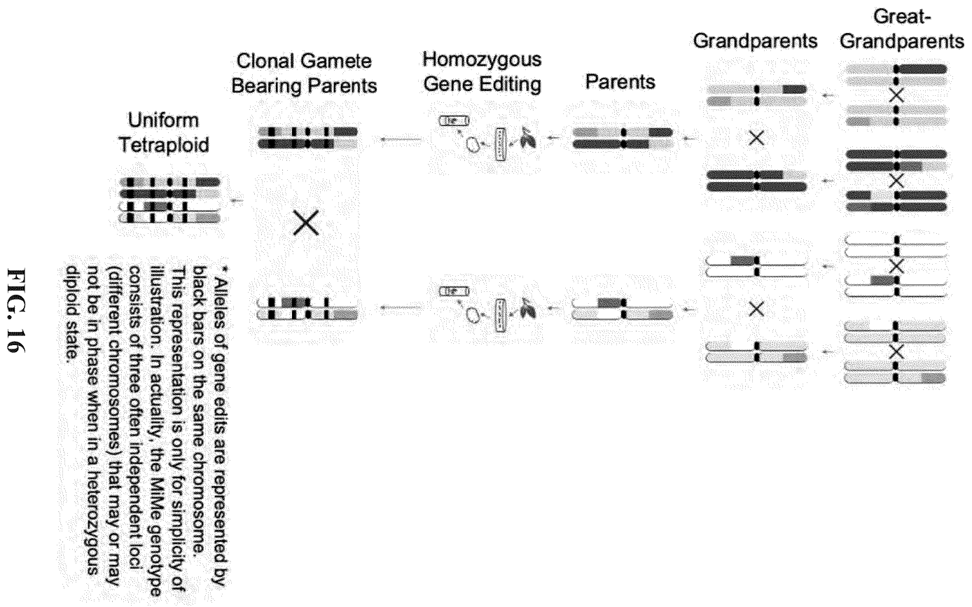

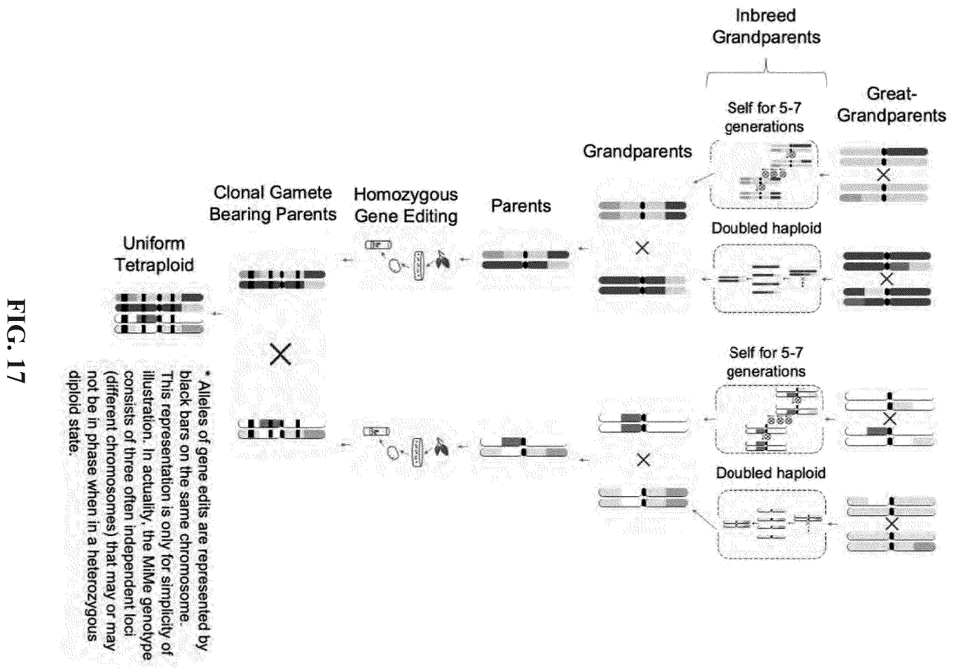

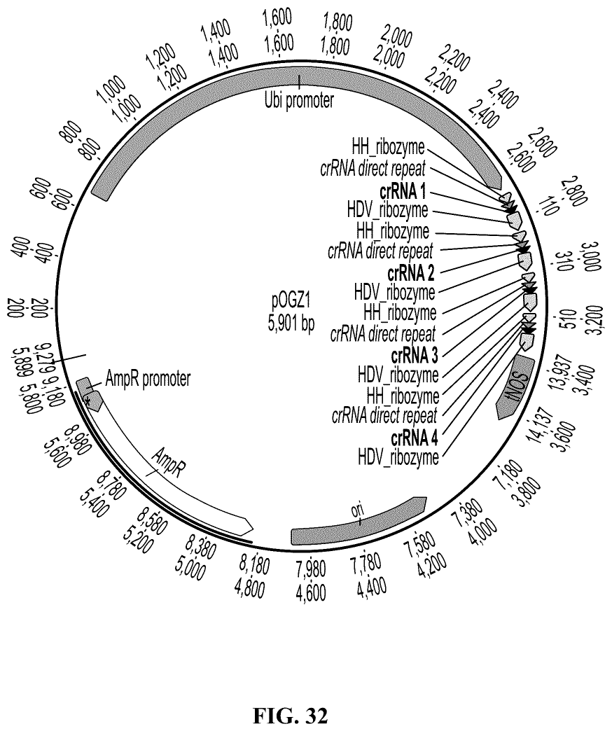

Plasmids were constructed for use in biolistic transformations of Zea mays. The most efficient crRNA guides were determined during guide screening in protoplasts, then guide constructs were designed placing those gRNAs between crRNA direct repeats or crRNA direct repeats and self-cleaving ribozyme sequences. These sequences were then synthesized and cloned into standard vectors. Plasmids encoding crRNA guides (“guide constructs”) included pOGZ2 (FIG. 14), pMEM6b (FIG. 13) and pOGZ1 (FIG. 32). Plasmids encoding a Cas nuclease (“nuclease construct”) were also synthesized including plasmids pLDB3 (FIG. 9), pLDB11B (FIG. 10), pLDB12B (FIG. 11), pMEM4 (FIG. 12) and pOGZ4 (FIG. 15). The components for each of these plasmids are listed in Table 13. For example, when transforming the combination of the guide construct pOGZ1 (FIG. 32) and nuclease encoding plasmid pMEM4 (FIG. 12), the MiMe loci which were targeted for editing were OSD1 (OSD1-1 and OSD1-2), SPO11, and REC8. Additional plasmids were constructed to target OSD1-1, OSD1-2, OSD1-3, and combinations thereof.

Using this approach, the guide constructs shown in Table 13 can be readily modified to target any combination of MiMe loci that can result in a partial or complete MiMe genotype including, for example, REC8, OSD1-1, OSD1-2, OSD1-3, PAIR1-1, PAIR1-2, SPO11-1, JASON-1, JASON-2, CYCA1-1, CYCA1-2, TDM1, and/or PS1. Additionally, the separated guide construct and nuclease construct can be transformed into separate lines to achieve parent MiMe plants with various crossing schemes as described further below in section ZM9, or the guide construct and nuclease construct can be co-transformed into the same line to directly achieve a MiMe genotype (the “TO approach”).

TABLE 13
Components of the plasmids used in biolistic experiments.

| | | Vector backbone components for |
|---|---|---|
| Plasmid | For plant expression | multiplication in E. coli |
| pLDB3 (FIG. 9) | ZmUBI: Maize Ubiquitin promoter | ori: origin of replication |
| | CAS: coding sequence of the selected | AmpR promoter: Ampicillin |
| | Cas nuclease | resistance promoter |
| | tHSP: terminator sequence from | AmpR: Ampicillin resistance |
| | Arabidopsis thaliana Heat shock | |
| | protein 18.2 | |
| | OsU6: RNA polymerase III promoter | |
| | from Oryza sativa | |
| | crRNA multiplex cassette containing | |
| | 4 crRNAs targeting REC8, OSD1-1, | |
| | OSD1-2, and SPO11 with poly-T | |
| | termination signal | |
| | CaMV35S promoter: constitutive | |
| | promoter derived from the | |
| | Cauliflower mosaic virus | |
| | BlpR: Bialaphos resistance gene | |
| | CaMV poly(A) signal: terminator | |
| | from Cauliflower mosaic virus | |
| pLDB11B (FIG. 10) | ZmUBI: Maize Ubiquitin promoter | ori: origin of replication |
| | CAS: coding sequence of the selected | AmpR promoter: Ampicillin |
| | Cas nuclease | resistance promoter |
| | tHSP: terminator sequence from | AmpR: Ampicillin resistance |
| | Arabidopsis thaliana Heat shock | |
| | protein 18.2 | |
| | CaMV35S: constitutive promoter | |
| | derived from the Cauliflower mosaic | |
| | virus | |
| | BlpR: Bialaphos resistance gene | |
| | CaMV poly(A) signal: terminator | |
| | from Cauliflower mosaic virus | |
| pLDB12B (FIG. 11) | OsU6: RNA polymerase III promoter | ori: origin of replication |
| | from Oryza sativa. | |
| | crRNA multiplex cassette containing | AmpR promoter: Ampicillin |
| | 4 or more crRNAs targeting REC8, | resistance promoter |
| | OSD1-1, OSD1-2, and SPO11 with | |
| | poly-T termination signal | |
| | tHsp: terminator sequence from | AmpR: Ampicillin resistance |
| | Arabidopsis thaliana Heat shock | |
| | protein 18.2 | |
| | CaMV35S: constitutive promoter | |
| | derived from the Cauliflower mosaic | |
| | virus | |
| | BlpR: Bialaphos resistance gene | |
| | CaMV poly A term: terminator from | |
| | Cauliflower mosaic virus | |
| pMEM4 (FIG. 12) | ZmUbi promoter: Maize Ubiquitin | ori: origin of replication |
| | promoter | |
| | Cas Nuclease: coding sequence of the | AmpR promoter: Ampicillin |
| | selected Cas nuclease | resistance promoter |
| | AtHSP Term: terminator sequence | AmpR: Ampicillin resistance |
| | from Arabidopsis thaliana Heat | |
| | shock protein 18.2 | |
| | LjUBI1 promoter: Lotus japonicus | |
| | polyubiquitin promoter | |
| | DsRed: red fluorescent protein | |
| | reporter gene | |
| | Nos terminator: Nopaline synthase | |
| | terminator from Agrobacterium | |
| | tumefaciens | |
| | CaMV35S promoter: constitutive | |
| | promoter derived from the | |
| | Cauliflower mosaic virus | |
| | BlpR: Bialaphos resistance gene | |
| | CaMV poly(A) signal: terminator | |
| | from Cauliflower mosaic virus | |
| pMEM6b (FIG. 13) | OsU6 Pro: RNA polymerase III | ColE1: origin of replication |
| | promoter from Oryza sativa | |
| | crRNA multiplex cassette containing | AmpR promoter: Ampicillin |
| | 4 crRNAs targeting REC8, OSD1-1, | resistance promoter |
| | OSD1-2, and SPO11 with poly-T | |
| | termination signal | |
| | LjUbi1 pro: Lotus japonicus | AmpR: Ampicillin resistance |
| | polyubiquitin promoter | |
| | GFP: green fluorescent protein | |
| | reporter gene | |
| | CaMV 35S Pro: constitutive | |
| | promoter derived from the | |
| | Cauliflower mosaic virus | |
| | BlpR: Bialaphos resistance gene | |
| | CaMV poly A term: terminator from | |
| | Cauliflower mosaic virus | |
| pOGZ1 (FIG. 32) | ZmUbi pro: Maize Ubiquitin | ori: origin of replication |
| | promoter | |
| | crRNA multiplex cassette containing | AmpR promoter: Ampicillin |
| | 4 or more crRNAs targeting REC8, | resistance promoter |
| | OSD1-1, OSD1-2, and SPO11 | |
| | HH_ribozyme and HDV_ribozyme: | AmpR: Ampicillin resistance |
| | non-coding RNA sequences that are | |
| | capable of self-cleavage | |
| | tNOS: Nopaline synthase terminator | |
| | from Agrobacterium tumefaciens | |
| pOGZ2 (FIG. 14) | Zm Ubi pro: Maize Ubiquitin | ColE1: origin of replication |
| | promoter | |
| | crRNA multiplex cassette containing | AmpR promoter: Ampicillin |
| | 4 or more crRNAs targeting REC8, | resistance promoter |
| | OSD1-1, OSD1-2, and SPO11 | |
| | HH_ribozyme and HDV_ribozyme: | AmpR: Ampicillin resistance |
| | non-coding RNA sequences that are | |
| | capable of self-cleavage | |
| | tNOS: Nopaline synthase terminator | |
| | from Agrobacterium tumefaciens | |
| | CaMV 35S: constitutive promoter | |
| | derived from the Cauliflower mosaic | |
| | virus | |
| | mCherryCDS: red fluorescent protein | |
| | reporter gene | |
| | Tvsp: Vegetative storage protein | |
| | terminator from Glycine Max | |
| pOGZ4 (FIG. 15) | 2 × 35S pro: 35S promoter with | ColE1: origin of replication |
| | duplicated enhancer region from | |
| | Cauliflower Mosaic Virus | |
| | BlpR: Bialaphos resistance gene | AmpR promoter: Ampicillin |
| | | resistance promoter |
| | CaMV poly A term: terminator from | AmpR: Ampicillin resistance |
| | Cauliflower mosaic virus | |
| | Zm Ubi pro: Maize Ubiquitin | |
| | promoter | |
| | Cas Nuclease: the chosenCas | |
| | nuclease | |
| | tNOS: Nopaline synthase terminator | |
| | from Agrobacterium tumefaciens | |

### ZM5: Plant Materials for Biolistic Transformation

Transformable diploid FFMM-AT6 and Hi-II were planted and grown with 16-hour/28° C. day and 8-hour/25° C. night cycles to produce ears containing immature embryos. Ears were harvested about 9 to 12 days after pollination and were surface sterilized using commercial bleach prior to performing embryo extraction as described by Wang et al. 2020 (Biolistic DNA delivery in maize immature embryos. Biolistic DNA Delivery in Plants: Methods and Protocols. pp. 177-195). Embryos were extracted and placed on either N6 Medium according to Wang and Frame 2009 (Wang, K. and Frame, B. (2009) Biolistic gun-mediated maize genetic transformation. Transgenic maize: methods and protocols. pp. 29-45) or 605T medium according to, for example, Jones et al. 2019 (Maize transformation using the morphogenic genes Baby Boom and Wuschel2. In Transgenic Plants. pp. 81-93. Humana Press, New York, NY) or, for example, Masters et al. 2020 (Agrobacterium Mediated Immature Embryo Transformation of Recalcitrant Maize Inbred Lines Using Morphogenic Genes. J. Vis. Exp. (156): e60782, doi:10.3791/60782). After that, embryos were incubated at 28° C. for 3 days. In some experiments, the incubation step described in the previous sentence was skipped and embryos were placed directly on N6 Osmotic Medium according to Wang et al. 2020.

### ZM6: Biolistic Mediated Transformation

Biolistic mediated transformation was performed according to Wang et al. 2020 with the modifications noted below. Bombardment was performed on the day of embryo extraction. Embryos were extracted to N6 osmotic medium (Wang et al. 2020) and incubated at 27° C. for 4 hours prior to bombardment. For bombardment, 125 ng plasmid DNA per shot was loaded onto 0.1 mg gold particles using 0.1 μL of a transfection reagent, TransIT®-2020, scaled to the number of shots. Gold particles were first mixed by pipetting with plasmid DNA in 50 μl sterile deionized water, then TransIT®-2020 was added and mixed by pipetting again. This mixture was incubated on ice for 10 minutes with gentle vortexing every 1 minute. Gold particles were centrifuged for 1 minute at 100×g and the supernatant was removed. Gold particles were washed with 50 ul of 100% ethanol, then resuspended in 7 μl of 100% ethanol per shot and loaded onto macrocarriers. Embryos were bombarded at 450 psi using a Bio-Rad Biolistic® PDS-1000 biolistic device.

### ZM7: Selection and Regeneration of Plants

For Hi-II callus, selection was performed according to Wang et al. 2020 with the modifications noted below. After bombardment, embryos were plated on N6 Selection I medium (1.5 mg/L bialaphos) and incubated at 27° C. in the dark for 2 weeks. After the 2-week incubation, callus was transferred to N6 Selection II (N6 SII) (3 mg/L bialaphos) and callus was further incubated for 2 weeks at 27° C. in the dark. Callus was transferred to fresh N6 SII medium every 2 weeks and checked for continual editing. Once sufficient editing was achieved, callus was moved directly to Regeneration II medium (Wang et al. 2020). Callus on Regeneration II medium was then incubated for 1 additional week at 27° C. in the dark, before being moved to a 16-hour day/8-hour night cycle in a lighted growth chamber with dim light and maintained at about 27° C. After 5 days of acclimation to the dim light, the light intensity was increased to about 100 ppfd light.

Once leafy shoots appeared, they were transferred to MS medium with 2 mg/L bialaphos as described in Wang et al. 2020. Once roots grew to a total length of >5 cm, the plantlets were transferred to soil. Leaf material was extracted and Illumina sequencing was performed to identify loss-of-function edits. Single plants were screened using the same technique as described in Example 1's section GM3 to identify individual events containing bi-allelic or mono-allelic conformations of each target gene including, for example, REC8, OSD1-1, OSD1-2, OSD1-3, PAIR1-1, PAIR1-2, SPO11-1, JASON-1, JASON-2, CYCA1, TDM1, PS1, or any combination thereof.

### ZM8: Plant Growth and Crossing Conditions

Regenerated plants with confirmed edits in target MiMe alleles were transplanted from tissue culture into PRO-MIX HP substrate and acclimatized in a growth chamber with 16-hour, 25° C. day, and 8-hour, 20° C. night with 60% relative humidity and 16-hour day length. After two to three weeks, plants were transplanted to soil and grown under greenhouse conditions with a continued 16-hour daylength. Once plants produced flowers, they were outcrossed as male or female to desired lines to introgress the transgene or edited alleles.

### ZM9: Crossing

Crossing Scheme 1. In some experiments, the cross begins with a single rooted plant transformed with the nuclease construct (designated PED-MRN11) and a single rooted plant transformed with guide construct (designated PED-MRG12). PED-MRN11 can be backcrossed to inbred lines designated PED-MR-A and PED-MR-C, respectively. Hybrid plants containing the nuclease construct pLDB11 can be selected, further backcrossed, and then designated PED-A-MRN11 and PED-C-MRN11, respectively. Separately, PED-MRG11 can be backcrossed to inbred lines designated PED-MR-B and PED-MR-D, respectively. Hybrid plants containing the guide construct pLDB12 are selected, further backcrossed, and then designated PED-B-MRG12 and PED-D-MRG12, respectively. PED-A-MRN11 can then be crossed to PED-B-MRG12 and plants containing both the nuclease construct and the guide construct and a complete MiMe genotype comprising bi-allelic edits in the target MiMe loci selected and designated PED-AB-MiMe1. Similarly, PED-C-MRN11 can be crossed to PED-D-MRG12, and progeny plants containing both the nuclease construct and the guide construct and a complete MiMe genotype comprising homozygous or bi-allelic edits in the target MiMe loci selected and designated PED-CD-MiMe2. PED-AB-MiMe1 can then be crossed to PED-CD-MiMe2, resulting in a genetically uniform hybrid population of tetraploid maize with four haplotypes designated PED-ABCD-Tet1.

Crossing Scheme 2. In some experiments, the cross begins with two rooted plants, each bearing mono-allelic mutations in MiMe alleles and generated as discussed in Crossing Scheme 1, but by selecting for mono-allelic mutations in MiMe alleles (sometimes referred to here as a “HetMiMe” plant) instead of bi-allelic mutations. These plants are designated PED-MR2A or PED-MR2B respectively, grown to maturity, and then crossed to each of the inbred lines PED-MR-E, PED-MR-F, PED-MR-G, and PED-MR-H. Seeds from the resulting hybrids are screened for mono-allelic nonfunctional mutations (HetMiMe plants) as described in Example 1's section GM3, and plants are again grown to maturity. Plants with the mono-allelic edits can be identified and backcrossed to produce lines with the respective mono-allelic mutations in each respective inbred background designated as PED-E-HetMiMe, PED-F-HetMiMe, PED-G-HetMiMe, and PED-H-HetMiMe. From the plants in each of these HetMiMe populations, heterozygous knockout plants and lines are then screened and intercrossed to make PED-E-HetMiMe x PED-F-HetMiMe hybrids and PED-G-HetMiMe x PED-H-HetMiMe hybrids. These hybrids are then screened, and two individual plants bearing a complete MiMe genotype (comprising bi-allelic mutations in the MiMe alleles) are identified and designated PED-EF-MiMe1 and PED-GH-MiMe2. PED-EF-MiMe1 can then be crossed to PED-GH-MiMe2, resulting in a genetically uniform hybrid population of tetraploid maize with four haplotypes designated PED-EFGH-Tet1.

Crossing Scheme 3. In some experiments, the cross began with one rooted F1 hybrid designated PED-MR3A-JK, bearing mono-allelic mutations in some MiMe alleles and bi-allelic mutations in other MiMe alleles and generated as discussed in Crossing Scheme 1, but by selecting for this configuration of mutations in MiMe alleles (instead of selecting for bi-allelic mutations). PED-MR3A-JK was then grown to maturity, selfed, and the resulting progeny were screened for plants with full bi-allelic mutations in the MiMe alleles. Separately, an inbred diploid designated PED-MR3B-LL was doubled in ploidy by using colchicine in tissue culture. PED-MR3A-JK was then crossed to PED-MR3B-LL, resulting in a genetically uniform hybrid population of tetraploid maize with three haplotypes designated PED-JKLL-Tet1.

### ZM10: Genotyping

High-quality genomic DNA was extracted from maize plants using Qiagen Dneasy Plant Mini kits (QIAGEN, Germantown, MD, USA). Purity was determined using a spectrophotometer, Nanodrop™ One-C (Thermo Fisher), and quantified with a fluorometer, Qubit™ Flex Fluorometer (Thermo Fisher). Samples were adjusted to a final concentration of 20 ng/μL. Markers were established and populations were genotyped as described in GM6. Alternatively, maize plants may be genotyped using targeted Genotyping By Sequencing (tGBS) (Ott et al. (2017) tGBS® genotyping-by-sequencing enables reliable genotyping of heterozygous loci. Nucleic acids research 45(21): e178-e178.).”

### ZR: Results

### ZR1: Guide Screening

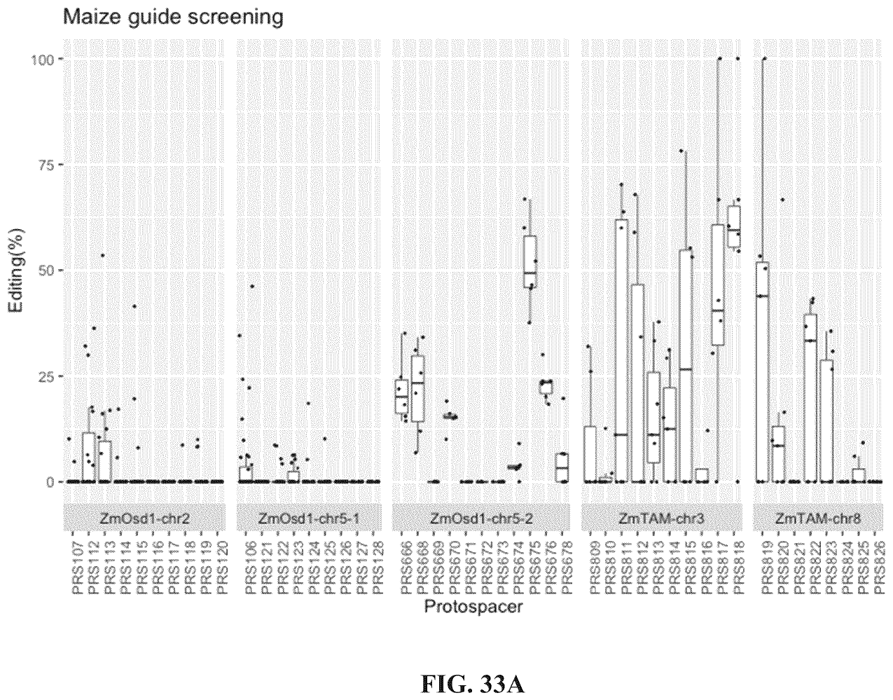

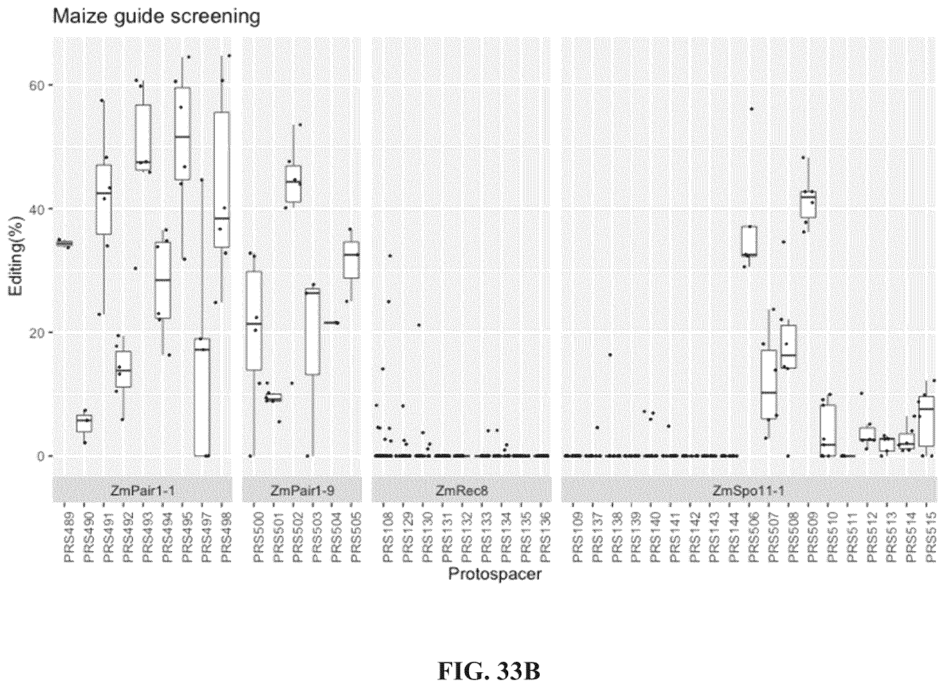

Guide RNAs were screened by RNP transfection or plasmid transfection as described in section ZM3. For maize protoplasts, a total of 10 guide RNAs were screened that targeted conserved regions of OSD1-1 (Table 14, SEQ ID NOs: 230-239). A total of 9 guide RNAs were screened that targeted conserved regions of OSD1-2 (Table 14, SEQ ID NOs: 240-248). A total of 9 guide RNAs were screened that targeted conserved regions of REC8 (Table 14, SEQ ID NOs: 249-257). A total of 19 guide RNAs were screened that targeted conserved regions of SPO11-1 (Table 14, SEQ ID NOs: 258-276). Editing efficiency was calculated by sequencing as described in Example 1's section GM3, and the resulting data presented in FIG. 33A and FIG. 33B. Based on this data, candidate guide RNAs were produced, as illustrated in Table 15. Guide RNA SEQ ID NO: 278 was chosen for editing OSD1-1; guide RNA SEQ ID NO: 287 was chosen for editing OSD1-2; guide RNA SEQ ID NO: 298 was chosen for editing REC8; guide RNA SEQ ID NO: 314 was chosen for editing Spo11-1.

TABLE 14
Sequences for conserved regions
of MiMe genes targeted in maize.

| SEQ ID NO. | PRSG# | Target DNA sequence |
|---|---|---|
| 230 | PRS107 | TGGTGGTGTTGGTGGTGGAG |
| 231 | PRS112 | TCAGGAGGGTGGCCTCGCCG |
| 232 | PRS113 | GGCGTTGAGGGTCACACCGA |
| 233 | PRS114 | TCGGTGTGACCCTCAACGCC |
| 234 | PRS115 | CAATGGCCGTAATATCACGG |
| 235 | PRS116 | ATCAGGAGGGTGGCCTCGCC |
| 236 | PRS117 | GTCGGTGTGACCCTCAACGC |
| 237 | PRS118 | CTGCAGAGCCCCGAGATGCC |
| 238 | PRS119 | CTGGATGGTACCCCAGGACG |
| 239 | PRS120 | GCAATGGCCGTAATATCACG |
| 240 | PRS106 | GAGGATGGCAAGCTGAAGAC |
| 241 | PRS121 | ACGATTGATGTGATGTCACG |
| 242 | PRS122 | CCTGACTGGTACCCGAGGAC |
| 243 | PRS123 | AAGATGCTTGAAGTGAGGAC |
| 244 | PRS124 | ACAGCACCATTCACCAGCAC |
| 245 | PRS125 | CCATCCTCCAAACAGGTTGC |
| 246 | PRS126 | AGCTTGCCATCCTCCAAACA |
| 247 | PRS127 | CCGATCTGTGGATCCAATAA |
| 248 | PRS128 | TCCTCCTCTCAATTGCCTGC |
| 249 | PRS108 | CGGTTGATCTTCGAGTGGAG |
| 250 | PRS129 | ATGATGTCGAGCTTGTCGAG |
| 251 | PRS130 | GAACCCCTCGGTGCCCATGG |
| 252 | PRS131 | TCGAGCCGTTTGCGGTTGAT |
| 253 | PRS132 | CTCTCGTACACGATCACCAC |
| 254 | PRS133 | ACCTTCCTCTCGTACACGAT |
| 255 | PRS134 | CTGTAGAGAAGCTTCACCTT |
| 256 | PRS135 | GAGTGGAGCGTCGCCGCCAT |
| 257 | PRS136 | TCGCGCGGAAGGCGCCGCTC |
| 258 | PRS109 | ATCCTGCGGAGGAGGAGCGC |
| 259 | PRS137 | TTCTGCTGCAGGAGCTGCTG |
| 260 | PRS138 | CTCCAGCCTCCGCCGCAACT |
| 261 | PRS139 | GATCCTGCGGAGGAGGAGCG |
| 262 | PRS140 | GCTCCCTGCAGAAGTAGCAG |
| 263 | PRS141 | AAGTGCAGCCGGCACAATCT |
| 264 | PRS142 | GAGCGGCGCCGCTCGTCGCC |
| 265 | PRS143 | AAGAGTATGCAGATATCGTT |
| 266 | PRS144 | GAGCAGTGCTTGTTCTGCTG |
| 267 | PRS506 | GATGTATTCGTACTGAGGCG |
| 268 | PRS507 | ATTGCCTCAATGTCAACAGG |
| 269 | PRS508 | CACATATCTTGGCTCCCTGC |
| 270 | PRS509 | GTGATGGGCTGGATAAGATT |
| 271 | PRS510 | TGGAGGGCGAAAAGAAAGTG |
| 272 | PRS511 | CCTTCTGTATATTCAGGCTT |
| 273 | PRS512 | TCCATTCCTGTTGACATTGA |
| 274 | PRS513 | TTCTCTTGCAGATGTTGTTA |
| 275 | PRS514 | CAGTGTTCCAGCGTTTGGCC |
| 276 | PRS515 | ACAGAACTTGTCATTGGCCA |

TABLE 15
Sequences for guide RNAs
targeting MiMe genes in maize.

| SEQ ID NO. | PRSG# | Guide RNA sequence |
|---|---|---|
| 277 | PRS107G | UGGUGGUGUUGGUGGUGGAG |
| 278 | PRS112G | UCAGGAGGGUGGCCUCGCCG |
| 279 | PRS113G | GGCGUUGAGGGUCACACCGA |
| 280 | PRS114G | UCGGUGUGACCCUCAACGCC |
| 281 | PRS115G | CAAUGGCCGUAAUAUCACGG |
| 282 | PRS116G | AUCAGGAGGGUGGCCUCGCC |
| 283 | PRS117G | GUCGGUGUGACCCUCAACGC |
| 284 | PRS118G | CUGCAGAGCCCCGAGAUGCC |
| 285 | PRS119G | CUGGAUGGUACCCCAGGACG |
| 286 | PRS120G | GCAAUGGCCGUAAUAUCACG |
| 287 | PRS106G | GAGGAUGGCAAGCUGAAGAC |
| 288 | PRS121G | ACGAUUGAUGUGAUGUCACG |
| 289 | PRS122G | CCUGACUGGUACCCGAGGAC |
| 290 | PRS123G | AAGAUGCUUGAAGUGAGGAC |
| 291 | PRS124G | ACAGCACCAUUCACCAGCAC |
| 292 | PRS125G | CCAUCCUCCAAACAGGUUGC |
| 293 | PRS126G | AGCUUGCCAUCCUCCAAACA |
| 294 | PRS127G | CCGAUCUGUGGAUCCAAUAA |
| 295 | PRS128G | UCCUCCUCUCAAUUGCCUGC |
| 296 | PRS108G | CGGUUGAUCUUCGAGUGGAG |
| 297 | PRS129G | AUGAUGUCGAGCUUGUCGAG |
| 298 | PRS130G | GAACCCCUCGGUGCCCAUGG |
| 299 | PRS131G | UCGAGCCGUUUGCGGUUGAU |
| 300 | PRS132G | CUCUCGUACACGAUCACCAC |
| 301 | PRS133G | ACCUUCCUCUCGUACACGAU |
| 302 | PRS134G | CUGUAGAGAAGCUUCACCUU |
| 303 | PRS135G | GAGUGGAGCGUCGCCGCCAU |
| 304 | PRS136G | UCGCGCGGAAGGCGCCGCUC |
| 305 | PRS109G | AUCCUGCGGAGGAGGAGCGC |
| 306 | PRS137G | UUCUGCUGCAGGAGCUGCUG |
| 307 | PRS138G | CUCCAGCCUCCGCCGCAACU |
| 308 | PRS139G | GAUCCUGCGGAGGAGGAGCG |
| 309 | PRS140G | GCUCCCUGCAGAAGUAGCAG |
| 310 | PRS141G | AAGUGCAGCCGGCACAAUCU |
| 311 | PRS142G | GAGCGGCGCCGCUCGUCGCC |
| 312 | PRS143G | AAGAGUAUGCAGAUAUCGUU |
| 313 | PRS144G | GAGCAGUGCUUGUUCUGCUG |
| 314 | PRS506G | GAUGUAUUCGUACUGAGGCG |
| 315 | PRS507G | AUUGCCUCAAUGUCAACAGG |
| 316 | PRS508G | CACAUAUCUUGGCUCCCUGC |
| 317 | PRS509G | GUGAUGGGCUGGAUAAGAUU |
| 318 | PRS510G | UGGAGGGCGAAAAGAAAGUG |
| 319 | PRS511G | CCUUCUGUAUAUUCAGGCUU |
| 320 | PRS512G | UCCAUUCCUGUUGACAUUGA |
| 321 | PRS513G | UUCUCUUGCAGAUGUUGUUA |
| 322 | PRS514G | CAGUGUUCCAGCGUUUGGCC |
| 323 | PRS515G | ACAGAACUUGUCAUUGGCCA |

### ZR2: Transformation and Selection of Regenerated Plants

To produce the first parent MiMe plant, a 2n Hi-II AxB Hybrid mother plant was crossed by pollen from a 2n Fast-Flowering Mini-Maize AT6 (FFMM-AT6) father. 14 days after pollination, the ear was harvested and placed at 4° C. overnight. The next morning, embryos were extracted, plated, and bombarded according to section ZM6 using the Bio-Rad Biolistic® PDS-1000 and 0.6 μm gold particles coated with the guide construct pOGZ1 and the nuclease construct pMEM4 loaded in a 3:1 molar ratio, respectively. Plasmid and gold were mixed in 50 μL sterile water, then a transfection reagent, TransIT®-2020, was added to bind the plasmid to the gold. Each gene gun shot was prepared with 125 ng DNA, 0.1 mg 0.6 μm gold particles, and 0.1 μL TransIT®-2020, and the gold particles were resuspended in 7 μL 100% ethanol and loaded onto macrocarriers, allowed to dry, then fired into the immature zygotic embryos at 450 psi. Gene gun settings were set according to Wang 2020. Plant PED-MN-HetMiMe was selected with a mono-allelic 10 bp frameshift-inducing mutation in osd1-1, bi-allelic frameshift-inducing 13 bp and 17 bp mutations in osd1-2, a 12 bp mutation in rec8 that disrupted a highly conserved coding region, and a mono-allelic 7 bp frameshift-inducing mutation in spo11. The plant was regenerated from tissue culture, grown in the greenhouse, then self-pollinated to produce T1 F2 seed population PED-MN-SPP. This seed was germinated, and a parent MiMe plant PED-MN-MiMe having bi-allelic loss-of-function mutations at osd1-2 and homozygous loss-of-function mutations at rec8 and spo11 was selected. PED-MN-MiMe showed fertility restoration and shed viable pollen. No edits of osd1-1 could be recovered from PED-MN-SPP.

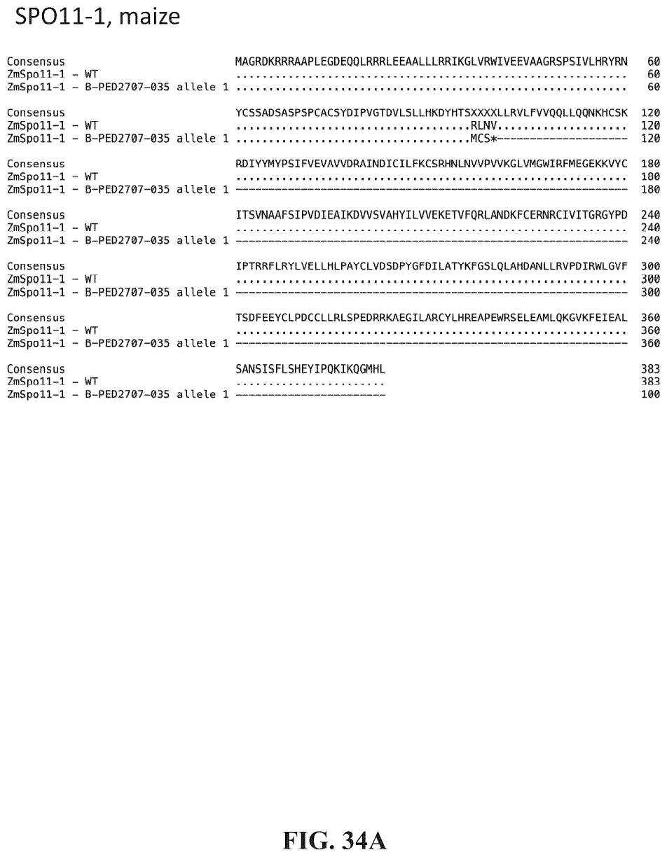

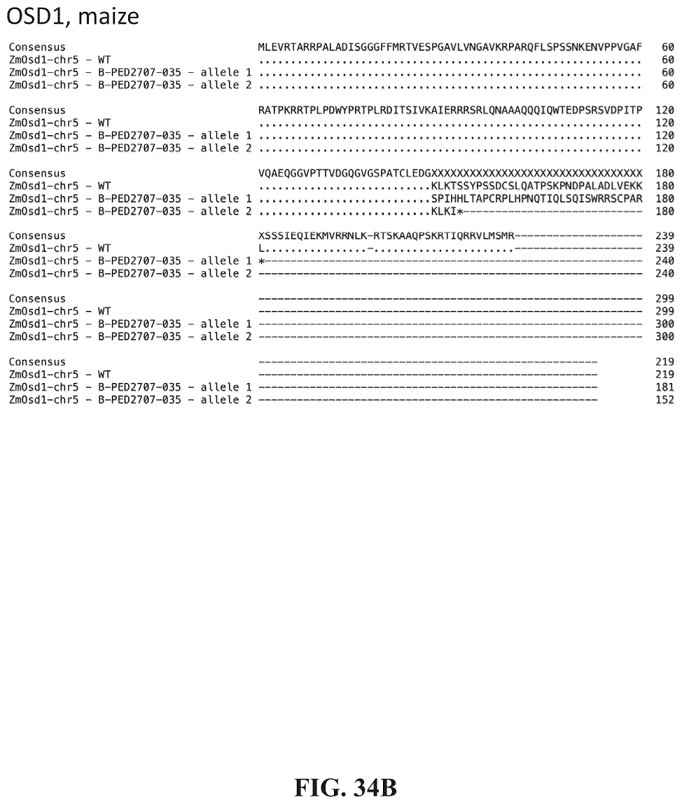

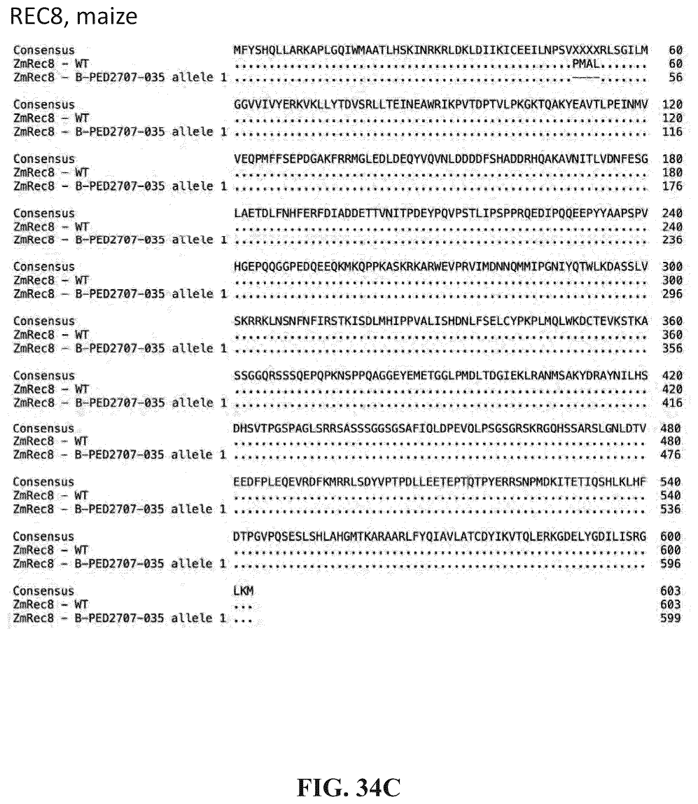

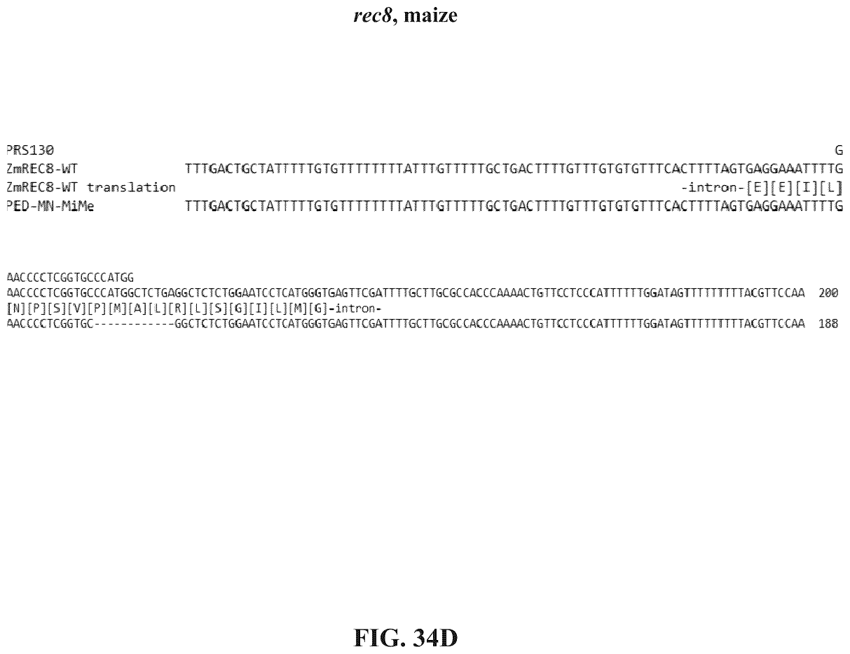

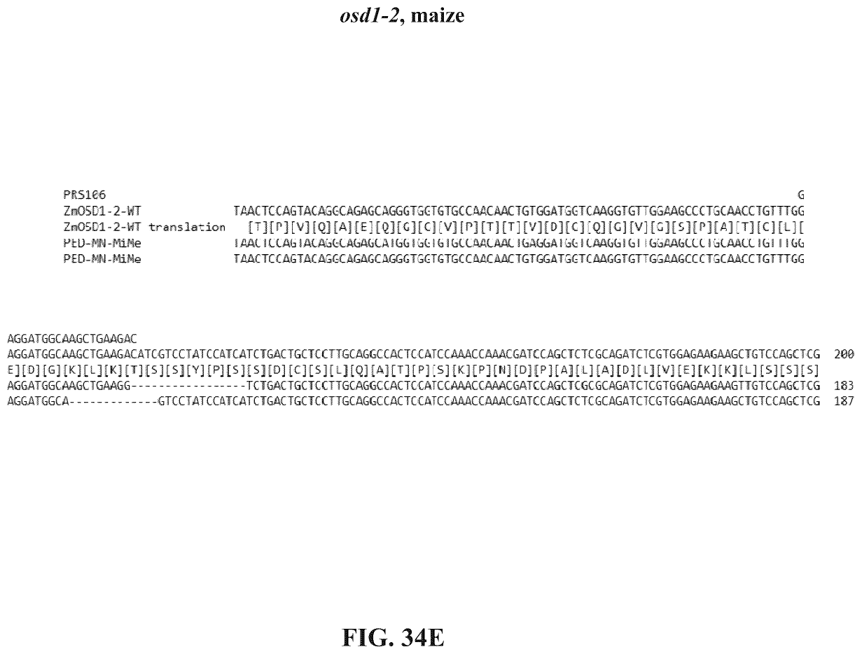

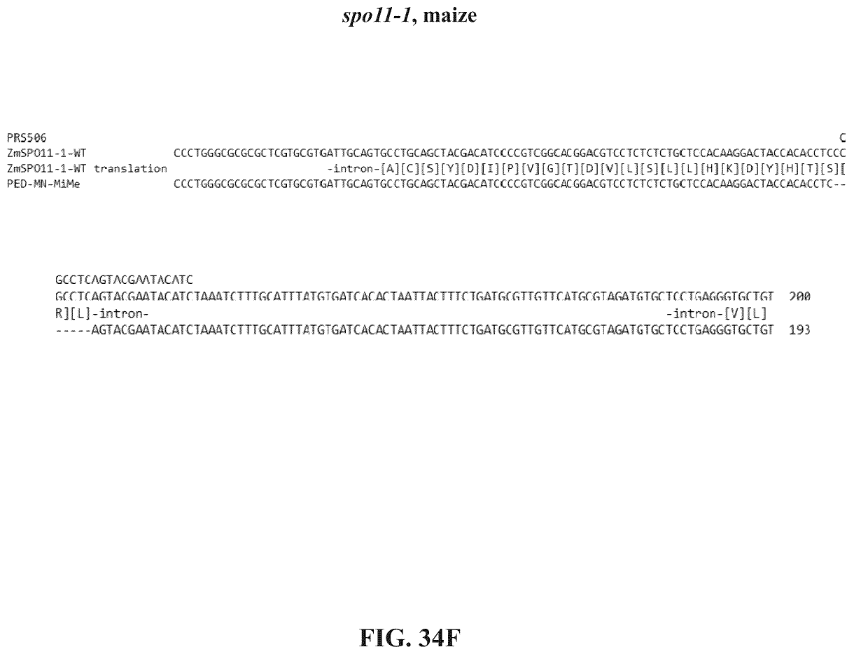

As an example, edits to the parent MiMe plant PED-MN-MiMe (rec8/osd1-2/spo11-1) are illustrated in FIGS. 34A-34C. In that figure, the predicted protein sequences are illustrated with respect to the unedited wildtype protein sequence and a consensus sequence, wherein an “X” in the consensus sequence indicates that there is no consensus for that amino acid position; wherein a dot (“.”) in the parent MiMe plant sequence indicates that the respective amino acid matches the consensus sequence; and wherein a dash (“-”) in the parent MiMe plant sequence indicates that the respective amino acid is present in the consensus sequence but is absent in the parent MiMe plant sample sequence. The deletion in REC8 disrupts a conserved protein sequence motif (FIGS. 34A and 34D). Frameshift inducing deletions in both alleles of osd1-2 (FIGS. 34B and 34E) and spo11-1 (FIGS. 34C and 34F) result in premature stop codons. Premature stop codons are denoted by an asterisk (“*”) in FIGS. 34B-34C.

### ZR3: Crossing and Seed Extraction

Pollen from PED-MN-MiMe (osd1-2/rec8/spo11) was collected and crossed to female PED-OO-4n plants, where PED-OO-4n was an inbred 4n tetraploid LH244, resulting in the Boosted Maize Population (“BMP”). Ears were harvested approximately 21-22 days after pollination, and BMP embryos were rescued to a non-selective MS rooting medium to germinate in the dark at 27° C. Once germinated, the plants were moved to rooting medium in a lighted incubator. Once rooted, the plants were moved to pots with PRO-MIX HP and covered with a humidity dome to acclimate before being transplanted to soil in the greenhouse.

### ZR4: Genotypic Evaluation

DNA samples were sequenced by Novogene. Libraries were prepared using the NEBNext® Ultra™ II for DNA Library Prep kit and paired-end 2×150 reads were sequenced on an Illumina NovaSeq 6000 or a NovaSeq X Plus. Markers were established and populations were genotyped as described in GM6.

For the parent MiMe plant PED-MN-MiMe and parent non-MiMe PED-OO-4n, DNA samples were sequenced to a depth of 120,987,086,400 bp, and 104,384,408,100 bp, respectively. For the BMP progeny plants, DNA samples were sequenced to an average depth of 96,945,478,675 bp.

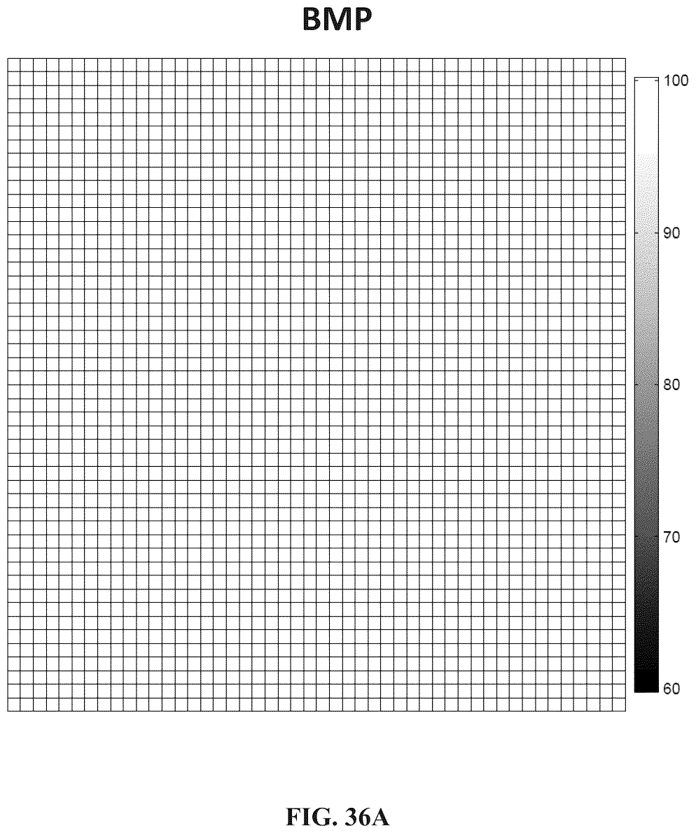

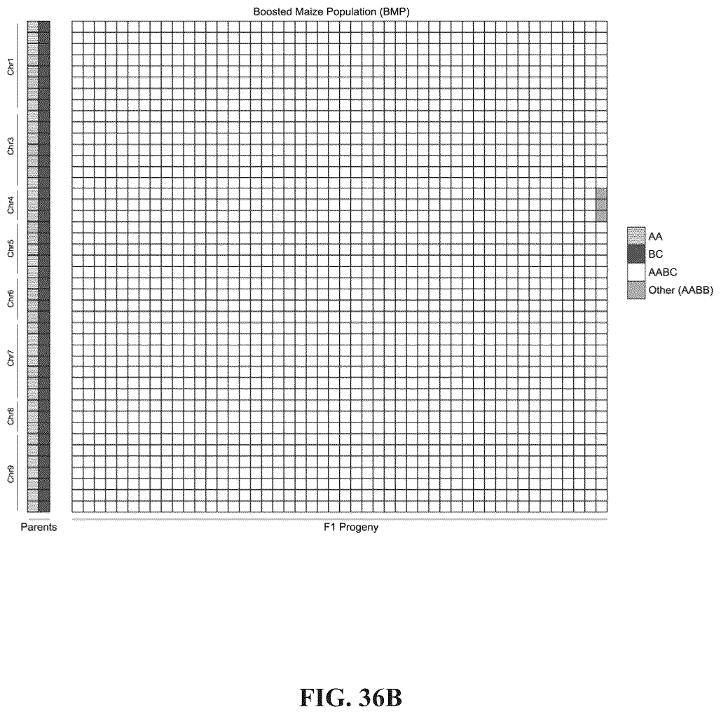

After filtering for high quality markers, a genotype matrix was assembled for BMP (FIG. 36B). Across the BMP population, the average pairwise genetic uniformity as measured by the Jaccard similarity coefficient was 99.9% (visualized as a pairwise matrix in FIG. 36A). This data is illustrated by the population uniformity graph in FIG. 35.

### ZR5: Phenotypic Evaluation of MiMe Progeny

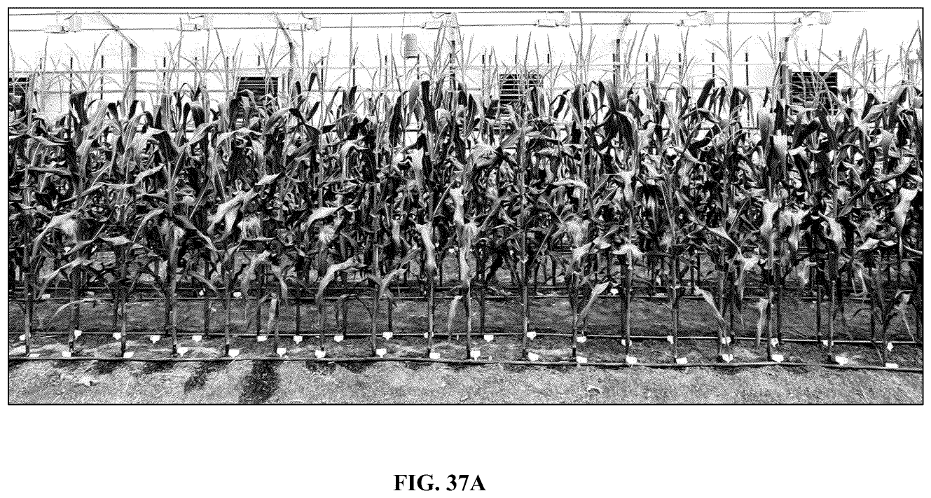

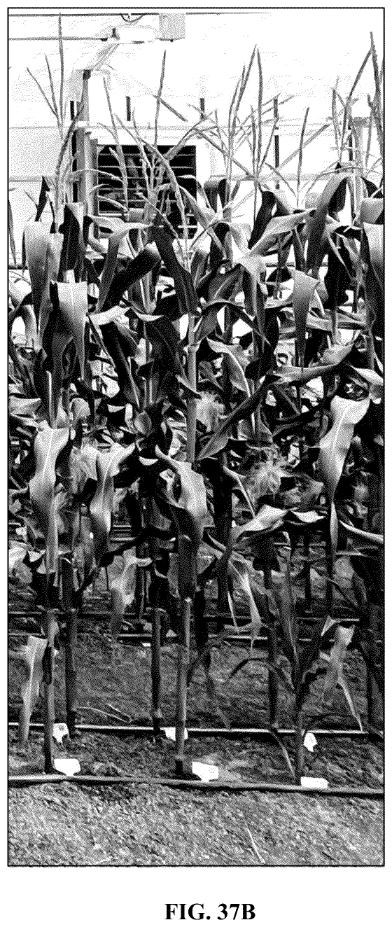

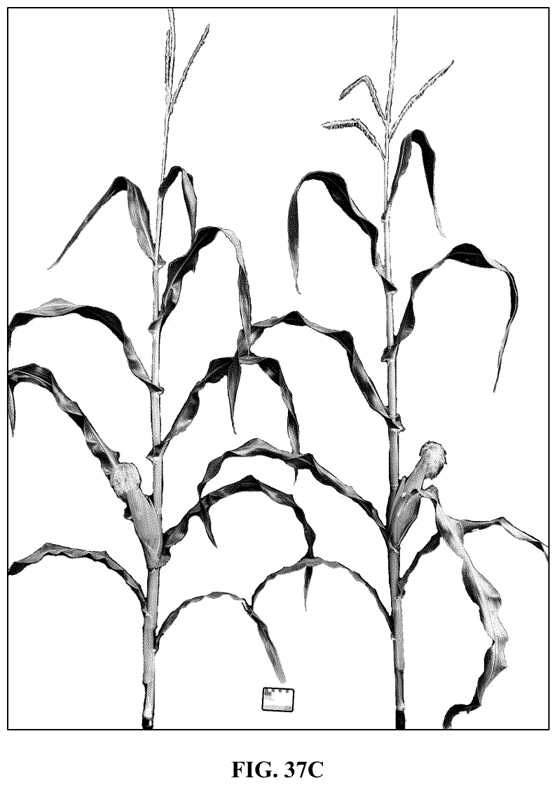

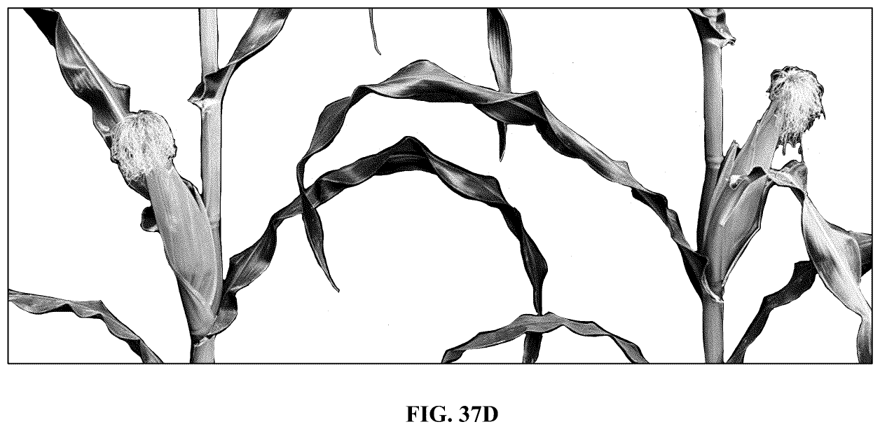

The BMP plants demonstrated a phenotypically uniform size, growth, and appearance, whereas typically the progeny of an F1 hybrid of non-inbred lines would show segregational variance. Maize plants from BMP are shown in FIG. 37A. All plants are 68 days post-embryo rescue and 57 days post-transplant from tissue culture to soil. A close-up of the right-hand side of FIG. 37A is shown in FIG. 37B, showing a close-up view of three BMP plants. FIG. 37C shows healthy BMP plants readily producing fruit, which are further displayed as a close-up view of the ears in FIG. 37D.

### Example 6: Uniform Polyploid Hybrid Banana (Musa) with Three or More Haplotypes

### MM: Methods

### MM1: Plant Materials and Protoplast Isolation

Diploid embryogenic cell suspension cultures of Musa acuminata with pedigree ID MUS002PAIR and pedigree ID MUS002SPO, triploid embryogenic cell suspension cultures of Musa acuminata with pedigree ID MUS003, and tetraploid embryogenic cell suspension cultures of Musa acuminata with pedigree ID MUS004 are initiated as described by Dhed'a et al. (1991. Plant regeneration in cell suspension cultures of the cooking banana cv. Bluggoes' (Musa spp. ABB group). Fruits. 46 (2): 125-135). Cultures are maintained using minor modifications as described by Panis et al (1993. Plant regeneration through direct somatic embryogenesis from protoplasts of banana (Musa spp.). Plant cell reports. 12(7): 403-407.). Cell cultures are subcultured every two weeks and kept under continuous cool white fluorescent light at 27° C. Two weeks before use in protoplast isolation, cells are transferred to fresh medium and are placed in complete darkness under the same conditions. An additional subculture is performed 3-5 days prior to use in protoplast extraction. Approximately one cubic centimeter of cells are harvested under aseptic conditions and are either immediately placed in enzyme solution or stored in a preconditioning medium for 1-24 hours before placement in enzyme solution. Other diploid, triploid, or tetraploid Musa acuminata, Musa balbisiana, or other Musa spp. are also selected and treated in the same manner.

To initiate protoplast isolation, approximately one cubic centimeter of cells are placed in 6-25 mL of enzyme solution containing 10 g/L Cellulase RS (Yakult, Tokyo, 105-8664 JAPAN) and 1.5 g/L Pectolyase Y-23 (Gold Bio, St Louis MO) as described by Panis et al. (1993). This cell suspension is placed on a rotary shaker at 40 RPM in the dark for between 7 and 24 hours. After digestion the resulting protoplasts are harvested through 100 μm sterile cell filters into sterile 50 mL conical tubes and are centrifuged at 100×G for 5 minutes. Supernatant is removed and replaced with 2 mL wash solution. Cells are washed twice by resuspending in the wash solution, then centrifuging at 100×G and removing the supernatant. After washing, cells are gently resuspended by rocking and are slowly layered onto a 0.43 M sucrose solution. Tubes are centrifuged at 100×G for 15 minutes. After fifteen minutes a thick dark band of protoplasts appears at the interface of the two solutions. This band is harvested in one continuous motion using a sterile serological pipette and combined with 5 mL of transformation buffer. Cells are quantified using a Bürker hemocytometer and stored at 4° C. in the dark until transfection. A sample is reserved to test cell viability using FDA staining as described by Larkin (1976. Purification and viability determinations of plant protoplasts. Planta. 128(3): 213-216).

### MM2: Transfection

Highly viable protoplasts are centrifuged at 50×g for 10 minutes and resuspended in a volume of transformation buffer to achieve a cell density of 1×10⁶ protoplasts/mL. Twenty microliters of freshly prepared RNP as described in section GM2 are added to the bottom of a 15 mL round bottom tube and 100 μL of protoplasts suspended in the transformation buffer are mixed with the RNP solution. Next, 120 μL of PEG solution as described in Wu et al. (2020. Establishment of a PEG-mediated protoplast transformation system based on DNA and CRISPR/Cas9 ribonucleoprotein complexes for banana. BMC Plant Biology 20, no. 1: 1-10.) is added and gently mixed by rotating the tubes. After a 30-minute incubation at room temperature the protoplasts are washed twice using a wash solution consisting of 0.55 M D-Mannitol, 15 mM CaCl₂, and 5 mM HEPES followed by centrifugation at 50×G for 10 minutes and removal of supernatant. A final centrifugation is performed and transfected cells are resuspended into suspension culture medium containing 10% mannitol and devoid of growth regulators as described by Panis et al (1993).

In a first set of experiments, the triploid embryogenic cell suspension cultures of Musa acuminata with pedigree ID MUS003, and the tetraploid embryogenic cell suspension cultures of Musa acuminata with pedigree ID MUS004 are transfected with RNPs targeting REC8, OSD1, and PAIR1 and/or SPO11-1.

In a second set of experiments, the diploid embryogenic cell suspension cultures of Musa acuminata with pedigree ID MUS002PAIR and pedigree ID MUS002SPO are transfected with RNPs targeting different gene sets. MUS002PAIR is transfected with RNPs designed to knock out REC8, ODS1 and/or CYCA1, and PAIR1. MUS002SPO is transfected with RNPs designed to knock out REC8, OSD1 and/or CYCA1, and Spo11-1.

### MM3: Encapsulation and Regeneration of Plants

Transfected protoplasts in suspension culture medium containing 10% mannitol and devoid of growth regulators is mixed with an equal volume of 3.2% sodium alginate. The resulting solution is pulled into a syringe and dispensed dropwise through a 23-gauge needle into 0.05M CaCl₂ solution containing 0.55 M D-Mannitol and 5 mM HEPES. Beads are incubated for 30 minutes at room temperature and are then washed with a suspension culture medium containing 0.55 M D-Mannitol and devoid of growth regulators.

Plant regeneration is roughly based on Panis et al. (1993) and Haicour et al. (2009. Protoplast isolation and culture for banana regeneration via somatic embryogenesis. Fruits. 64(4): 261-269) and is briefly summarized as follows. After encapsulation alginate beads are transferred to a solid medium containing nurse cells as described by Haicour et al. (2009) and 0.3 to 1 mL of liquid N6 culture medium is added to facilitate nutrient mobility. Beads are grown in the dark at 27° C. for 17-25 days and 0.3 to 1 mL of liquid N6 culture medium is added to the plate weekly during this interval. After 3-4 weeks of culture, proembryos are observed and are liberated from alginate beads using a citric acid solution consisting of 20 mM Sodium Citrate and 0.5 M Sorbitol. Liberated pro-embryos are plated onto regeneration medium as described by Panis et al. (1993).

Developing proembryos are transferred to hormone-free plant-based culture media (PCM) as described by Haicour et al. (2009) medium with 30 g/L sucrose at 27° C. Light is gradually increased to 65 μmol per meter square per second over a one week period while maintaining a 12 hour day length. Plants are multiplied and genotyped using the same sequencing methods described in Example 1's section GM3. Plants containing complete MiMe or HetMiMe genotypes are further multiplied and rooted on hormone-free PCM medium with 30 g/L sucrose and 12-hour day.

### MM4: Plant Growth and Crossing Conditions

Regenerated plants with confirmed edits in each target gene including REC8, OSD1, and PAIR1 and/or SPO11 are pre-hardened off by exchanging unvented lids for vented lids. After two weeks of pre-hardening, rooted plants are removed from tissue culture and transplanted into 2 parts Sunshine professional, one part perlite, and one part vermiculite. Plants are grown under greenhouse conditions at about 27° C. and supplemental light is gradually increased over 1 week. After two to three weeks plants are transplanted to 1-gallon pots and are grown under greenhouse conditions with a continued 12-hour daylength. After roots reach the bottom of the pot plants are grown directly in greenhouse soil. A homozygous diploid or doubled haploid Musa plant with pedigree MUS001 is acclimatized and grown under the same conditions.

Acclimatized and edited plants from MUS002SPO, MUS002PAIR, MUS003, and MUS004 are identified using methods described in section GM3. Knockouts of target genes corresponding to the transfected RNPs are designated MUS002spoE (rec8/rec8, osd1/osd1, and/or cyca1/cyca1, spo11/spo11), MUS002pairE (rec8/rec8, osd1/osd1 and/or cyca1/cyca1, pair1/pair1), MUS002spoHet (REC8/rec8, OSD1/osd1 and/or CYCA1/cyca1, SPO11/spo11), MUS002pairHet (REC8/rec8, OSD1/osd1 and/or CYCA1/cyca1, PAIR1/pair1), MUS003E (rec8/rec8/rec8, osd1/osd1/osd1 and/or cyca1/cyca1/cyca1, spo11/spo11/spo11 and/or pair1/pair1/pair1), and MUS004 (rec8/rec8/rec8/rec8, osd1/osd1/osd1/osd1 and/or cyca1/cyca1/cyca1/cyca1, spo11/spo11/spo11/spo11 and/or pair1/pair1/pair1/pair1). The homozygous diploid or doubled haploid Musa plant with pedigree MUS001 is screened in the same manner and is determined to be wild type for the genes of interest (REC8/REC8, OSD1/OSD1, CYCA1/CA SPO11/SPO11, PAIR1/PAIR1). In some plants multiple copies of certain target genes are identified and guides are designed to target these genes in isolation or in combination with one another. In the first set of experiments, when plants produce flowers (200-300 days after planting), pollen is collected from the male (MUS001, unedited) and cross pollination is performed. The cross resulting from MUS002spoE (female edited) and MUS001 (male) is designated MUS002spoE-001. The cross resulting from MUS003E (female edited) and MUS001 (male) is designated MUS003E-001. The cross resulting from MUS004E (female edited) and MUS001 (male) is designated MUS004E-001. In 80-120 days, fruits are harvested, and seeds are extracted with standard methods. Seeds are cleaned and dried in preparation for embryo rescue.

In the second set of experiments, when plants produce flowers (200-300 days after planting) the pollen is collected from MUS002spoE and MUS002pairE. Reciprocal crossing is performed resulting in a population designated MUS004spoE-pairE. In 80-120 days, fruits are harvested, and seeds are extracted with standard methods. Seeds are cleaned and dried in preparation for germination and/or embryo rescue.

In the third set of experiments, when plants produce flowers (200-300 days after planting) the pollen is collected from MUS002spoHet and MUS002pairHet and crossed to wild-type diploid Musa acuminata (MUS002WT). The cross resulting from MUS002spoHet and MUS002WT is designated MUS002spoBC, and the cross resulting from MUS002pairHet and MUS002WT is designated MUS002pairBC. In 80-120 days, fruits are harvested, and seeds are extracted with standard methods. Seeds are cleaned and dried in preparation for germination and/or embryo rescue.

Seed from this cross is initiated into a backcrossing program wherein individuals heterozygous for the MiMe genes are backcrossed to wildtype diploid lines, resulting in genotypes MUS002spoBCX-A and MUS002spoBCX-B (REC8/rec8, OSD1/osd1 and/or CYCA1/cyca1, SPO11/spo11), and MUS002pairBCX-C and MUS002pairBCX-D (REC8/rec8, OSD1/osd1 and/or CYCA1/cyca1, PAIR1/pair1). When plants of these genotypes produce flowers (200-300 days after planting) the pollen is collected from MUS002spoBCX-A and crossed to MUS002spoBCX-B to produce seed population MUS002spoBCP-AB. Similarly, pollen is collected from MUS002pairBCX-C and crossed to MUS002pairBCX-D to produce seed population MUS002pairBCP-CD.

Individuals from MUS002spoBCP-AB are screened to find a plant with a full MiMe genotype (rec8/rec8, osd1/osd1 and/or cyca1/cyca1, spo11/spo11) designated MUS002spoBCAB, similarly MUS002pairBCP-CD is screened to find a plant with a full MiMe genotype (rec8/rec8, osd1/osd1 and/or cyca1/cyca1, pair1/pair1) designated MUS002pairBCCD, and these plants are transplanted to the field. When plants produce flowers (200-300 days after planting) the pollen is collected from MUS002spoBCAB and used to pollinate MUS002pairBCCD, resulting in a population of seeds MUS002spoAB-pairCD (rec8/rec8/rec8/rec8, osd1/osd1/osd1/osd1 and/or cyca1/cyca1/cyca1/cyca1, spo11/spo11/SPO11/SPO11 and PAIR1/PAIR1/pair1/pair1). After 80-120 days, fruits are harvested, and seeds are extracted with standard methods. Seeds are cleaned and dried in preparation for germination and/or embryo rescue.

### MM5: Genotyping of Uniform Polyploid Hybrids

Following seed extraction, the 200 cleaned seeds are surface disinfested from each cross for 20 minutes in a sonic bath using a ten percent bleach solution containing 20 microliters of Tween 20 (Sigma) in a final volume of 50 mL. Disinfested seeds are rinsed twice with sterile deionized water and embryos are placed into hormone free PCM medium with 30 g/L sucrose and grown at 12-hour daylength. Plants are transferred every two weeks to fresh medium and leaves are removed for DNA extraction during this process.

High quality genomic DNA is extracted from leaves of 100 randomly selected plants from MUS002spoE-001, MUS002spoE-pairE, MUS003E-001, MUS004E-001, and MUS002spoAB-pairCD using Qiagen DNeasy Plant Mini kits (catalog number 69104) (QIAGEN, Germantown, MD, USA). Purity is determined using a spectrophotometer, Nanodrop™ One-C (ThermoFisher), and quantified on a fluorometer, Qubit™ Flex Fluorometer (Q33327). Samples are adjusted to a final concentration of 20 ng/μL and are genotyped using Rapid Genomics Flex-Seq™ Services (Gainesville, Florida, USA). Following a standard genotyping pipeline for autopolyploids the results are filtered for quality. Genotypic markers specific to the edited parent and indicative of unique haplotypes for MUS001, MUS002spoE, MUS002pairE, MUS003E, and MUS004E are identified and used to determine that the full complement of chromosomes was transmitted to each of the progeny.

### MM6: Hybrid Plant Growth and Pollen Viability

Plants resulting from embryo rescue or direct greenhouse seeding of populations MUS002spoE-001, MUS002spoE-pairE, MUS003E-001, MUS004E-001, and MUS002spoAB-pairCD are grown to maturity under standard greenhouse conditions along with clones of parents used to produce each population. After 200-300 days plants reach maturity and flower for the first time. Pollen collected in the early morning is stained using FDA as described by Heslop-Harrison, J, & Heslop-Harrison, Y. (1970Y Evaluation of pollen viability by enzymatically induced fluorescence; intracellular hydrolysis of fluorescein diacetate. Stain technology, 45(3), 115-120. to determine viability.

### MM7: Evaluation of Parthenocarpic Fruit Formation

Following pollen viability tests plants are allowed to undergo normal growth and development. The total number per fruit per plant and seeds per fruit is evaluated for members of MUS002spoE-001, MUS002spoE-pairE, MUS003E-001, and MUS004E-001 as well as the parents of each population. Fruits mature between 80 and 120 days after flowering and are examined for the presence of seeds.

### MR: Results

### MR1: Selection of Regenerated Plants

Acclimatized and edited plants derived from MUS002SPO, MUS002PAIR, MUS003, and MUS004 are identified using methods described in section GM3 and between one and ten percent of regenerated plants have mutations predicted to result in nonfunctional proteins of REC8, OSD1 and PAIR1 or in REC8, OSD1 and SPO11-1 or REC8, OSD1 and PAIR1 and SPO11-1. Out of between 2,000 and 10,000 regenerated plants approximately 20 to 100 unique plants are selected for evaluation in a partial diallel crossing scheme. Plants predicted to produce nonfunctional copies of target genes are designated MUS002spoE (rec8/rec8, osd1/osd1, spo11/spo11), MUS002pairE (rec8/rec8, osd1/osd1, pair1/pair1), MUS003E (rec8/rec8/rec8, osd1/osd1/osd1, spo11/spo11/spo11 and/or pair1/pair1/pair1), and MUS004E (rec8/rec8/rec8/rec8, osd1/osd1/osd1/osd1, spo11/spo11/spo11/spo11 and/or pair1/pair1/pair1/pair1).

### MR2: Crossing and Embryo Rescue Results

Seeds from MUS002spoE-001, MUS003E-001, and MUS004E-001 are embryo rescued and the success rate ranges per seed lot are from 10-40%. Some seeds have more developed endosperm and are candidates for direct germination without embryo rescue. MUS002spoAB-pairCD, MUS002spoE-pairE, and MUS004E-001 also produce a larger number of seeds per cross relative to MUS003E-001 and MUS002spoE-001. Each cross is evaluated further using flow cytometry.

Seeds from MUS002spoE-pairE and MUS002spoAB-pairCD have normal endosperm (similar to wild type seeded tetraploid bananas) and are embryo rescued. Approximately 30-85% of embryos result in viable plants. A portion of the seeds are also directly germinated resulting in 10-50% seed germination under standard greenhouse conditions.

### MR3: Flow Cytometry

Parent MUS001 is shown to be diploid having DNA content of approximately 1.1-1.3 pg per 2C nucleus. MUS002spoE is shown to be diploid having DNA content of approximately 1.1-1.3 pg per 2C nucleus. MUS002pairE is shown to be diploid having DNA content of approximately 1.1-1.3 pg per 2C nucleus. MUS003E is shown to be triploid having DNA content of approximately 1.6-1.9 pg per 2C nucleus. MUS004E is shown to be tetraploid having DNA content of approximately 2.2-2.6 pg per 2C nucleus relative to internal controls.

Parent MUS002pairBCCD is shown to be diploid having DNA content of approximately 1.1-1.3 pg per 2C nucleus. MUS002spoBCAB is shown to be diploid having DNA content of approximately 1.1-1.3 pg per 2C nucleus.

Greater than 70% of the 100 progeny of MUS002spoE (edited female) and MUS001 in family MUS002spoE-001 are shown to be triploid with DNA content of approximately 1.6-1.9 pg per 2C nucleus and individual measurements show similar variability between family members as observed in technical replicates from the same plant.

Greater than 70% of the 100 progeny of MUS003E (edited female) and MUS001 in family MUS003E-001 are shown to be tetraploid with DNA content of approximately 2.2-2.6 pg per 2C nucleus and individual measurements show similar variability between family members as observed in technical replicates from the same plant.

Greater than 70% of the 100 progeny of MUS004E (edited female) and MUS001 in family MUS004E-001 are shown to be pentaploid with DNA content of approximately 2.75-3.05 pg per 2C nucleus and individual measurements show similar variability between family members as observed in technical replicates from the same plant.

Greater than 70% of the 100 progeny of MUS002spoE and MUS002pairE and the reciprocal cross in family MUS002spoE-002pairE are shown to be tetraploid with DNA content of approximately 2.2-2.6 pg per 2C per nucleus and individual measurements show similar variability between family members as observed in technical replicates from the same plant.

Greater than 70% of the 100 progeny of MUS002spoBCAB and MUS002pairBCCD and the reciprocal cross in family MUS002spoAB-pairCD are shown to be tetraploid with DNA content of approximately 2.2-2.6 pg per 2C per nucleus and individual measurements show similar variability between family members as observed in technical replicates from the same plant.

### MR4: Genotypic Evaluation of Uniform Polyploid Hybrids

Genotypic matrices and haplotyping results show specific loci with three alleles in more than 70% of triploid members in MUS002E-001 and also show extended haplotype blocks with three haplotypes. After filtering for high quality markers, the genotyping matrices for the 100 progeny of the triploid family MUS002E-001 are greater than 70% similar.

Genotypic matrices and haplotyping results show specific loci with three or more alleles in more than 70% of tetraploid members in MUS003E-001 and also show extended haplotype blocks with three or more haplotypes. After filtering for high quality markers, the genotyping matrices for the 100 progeny of the tetraploid family MUS003E-001 are greater than 70% similar.

Genotypic matrices and haplotyping results show specific loci with greater than three alleles in more than 70% of pentaploid members in MUS004E-001 and also show extended haplotype blocks with three or more haplotypes. After filtering for high quality markers, the genotyping matrices for the 100 progeny of the pentaploid family MUS004E-001 are greater than 70% similar.

Genotypic matrices and haplotyping results show specific loci with greater than three alleles in more than 70% of tetraploid members in MUS002spoE-pairE and also show extended haplotype blocks with three or more haplotypes. After filtering for high quality markers, the genotyping matrices for the 100 progeny of the tetraploid family MUS002spoE-pairE are greater than 70% similar.

Genotypic matrices and haplotyping results show specific loci with greater than three alleles in more than 70% of tetraploid members in MUS002spoAB-pairCD and also show extended haplotype blocks with three or more haplotypes. After filtering for high quality markers, the genotyping matrices for the 100 progeny of the tetraploid family MUS002spoAB-pairCD are greater than 70% similar.

### MR5: Pollen Viability

Pollen from the diploid plant with pedigree ID MUS001 is collected at three time points on two separate days and the pollen viability ranges from about 30% to about 70%.

Pollen from the diploid plant with pedigree ID MUS002 is collected at three time points on two separate days and the pollen viability ranges from about 30% to about 70%.

Pollen from the triploid plant with pedigree ID MUS003 is collected at three time points on two separate days and the pollen viability is generally less than 1%.

Pollen from the tetraploid plant with pedigree ID MUS004 is collected at three time points on two separate days and the pollen viability ranges from about 30% to about 70%.

Pollen from the gene edited diploid plant with pedigree ID MUS002spoE (rec8/rec8, osd1/osd1, spo11/spo1l) is collected at three time points on two separate days and the pollen viability ranges from about 30% to about 70%.

Pollen from the gene edited diploid plant with pedigree ID MUS002pairE (rec8/rec8, osd1/osd1, pair1/pair1) is collected at three time points on two separate days and the pollen viability ranges from about 30% to about 70%.

Pollen from the gene edited diploid plant with pedigree ID MUS002spoHet (REC8/rec8, OSD1/osd1, SPO11/spo11) is collected at three time points on two separate days and the pollen viability ranges from about 30% to about 70%.

Pollen from the gene edited diploid plant with pedigree ID MUS002pairHet (REC8/rec8, OSD1/osd1, PAIR1/pair1) is collected at three time points on two separate days and the pollen viability ranges from about 30% to about 70%.

Pollen from the gene edited diploid plants with pedigree IDs MUS002spoBCX-A and MUS002spoBCX-B (REC8/rec8, OSD1/osd1, SPO11/spo11) is collected at three time points on two separate days and the pollen viability ranges from about 30% to about 70%.

Pollen from the gene edited diploid plants with pedigree IDs MUS002pairBCX-C and MUS002pairBCX-D (REC8/rec8, OSD1/osd1, PAIR1/pair1) is collected at three time points on two separate days and the pollen viability ranges from about 30% to about 70%.

Pollen from the gene edited diploid plant with pedigree ID MUS002spoBCAB (rec8/rec8, osd1/osd1, spo11/spo1) is collected at three time points on two separate days and the pollen viability ranges from about 30% to about 70%.

Pollen from the gene edited diploid plant with pedigree ID MUS002pairBCCD (rec8/rec8, osd1/osd1, pair1/pair1) is collected at three time points on two separate days and the pollen viability ranges from about 30% to about 70%.

Pollen from the gene edited triploid plant with pedigree ID MUS003E (rec8/rec8/rec8, osd1/osd1/osd1, spo11/spo11/spo11 or pair1/pair1/pair1) is collected at three time points on two separate days and the pollen viability ranges from about 30% to about 70%. High pollen viability in the edited triploid MUS003E relative to the unedited MUS003 is attributed to successful conversion of Meiosis to Mitosis.

Pollen from the tetraploid plant with pedigree ID MUS004E (rec8/rec8/rec8/rec8, osd1/osd1/osd1/osd1, spo11/spo11/spo11/spo11 or pair1/pair1/pair1/pair1) is collected at three time points on two separate days and the pollen viability ranges from about 30% to about 70%.

Pollen from the triploid population designated MUS002spoE-001 is collected at three time points on two separate days. Pollen viability is less than 1%. Low viability relative to the female parent is attributed to genetic complementation which restores the wild type meiosis and eliminates clonal gamete production.

Pollen from the tetraploid population designated MUS003E-001 is collected at three time points on two separate days. Pollen viability ranges from about 30% to about 70% and is similar to unedited tetraploid plants.

Pollen from the pentaploid population designated MUS004E-001 is collected at three time points on two separate days. Pollen viability is less than 1%. Low viability is attributed to genetic complementation which restores the wild type meiosis and eliminates clonal gamete production.

Pollen from the tetraploid population designated MUS002spoE-pairE is collected at three time points on two separate days. Pollen viability is less than 1%. Low viability is attributed to partial genetic complementation which eliminates clonal gamete production (rec8/rec8/rec8/rec8, osd1/osd1/osd1/osd1, SPO11/SPO11/spo11/spo11, pair1/pair1/PAIR1/PAIR1).

Pollen from the tetraploid population designated MUS002spoAB-pairCD is collected at three time points on two separate days. Pollen viability is less than 1%. Low viability is attributed to partial genetic complementation which eliminates clonal gamete production (rec8/rec8/rec8/rec8, osd1/osd1/osd1/osd1, SPO11/SPO11/spo11/spo11, pair1/pair1/PAIR1/PAIR1).

### MR6: Evaluation of Parthenocarpic Fruit

Fruit is examined in hybrid plant populations MUS002spoE-001, MUS003E-001, MUS004E-001, MUS002spoE-pairE, and MUS002spoAB-pairCD. MUS003E-001 exhibits fruits with fully developed normal seed. Parthenocarpic fruits form on all other plants and no fully developed seeds are observed.

### Example 7: Generating Genetically Uniform Tetraploid Hybrid Arabidopsis with Three or Four Haplotypes

### AM: Methods

### AM1: Plant Materials and Protoplast Isolation

Plants from Arabidopsis accessions that were selected and tested included Landsberg, Shahdara, HR-10, Ws-2, Col-0 (CS851557-S1), CS851294, Columbia (“Col-0”), Col-0 x Shahdara F1, Landsberg x Shahdara F1, Col-0 x Shahdara F2, Landsberg x Shahdara F2, Col-0 (CS851557-S1) x HR-10 F2, Col-0 x HR-10 F2, and Shahdara x Ws-2 F2.

Before germinating, seeds were surface sterilized using 70% ethanol solution for 5 min. Then, seeds were rinsed with sterile, distilled water five times. Seeds were stratified at 4° C. and then germinated on hormone-free AGM (Arabidopsis Germination Medium) containing half strength Murashige and Skoog basal salts, 10 g/L sucrose, 0.5 g/L MES and 8 g/L Agar (pH 5.8) and grown under 18-hour light/6-hour dark photoperiod (40-60 μmol photons m-2 s-1) at 23° C. Approximately 1 gram of leaves from each batch of 10-day old seedlings was removed under aseptic conditions for protoplast extraction.

For protoplast isolation, leaves were sliced into thin sections approximately 1 mm in width and incubated in an enzyme solution composed of 0.5% (weight/volume) Cellulase RS (Duchefa Biochemie, The Netherlands) and 0.5% (weight/volume) Macerozyme R10 (Duchefa Biochemie, The Netherlands) in MMC solution (10 mM MES, 0.47 M Mannitol, 10 mM Calcium Chloride) for a final volume of 10 mL. Sliced leaves in the digest solution were then incubated for around 15 hours at 24° C. in darkness. The protoplasts were then liberated from the leaf tissue with 15-minute shaking at 40 RPM at room temperature.

Protoplasts were then diluted with equal volume of At-W5 buffer (154 mM NaCl, 125 mM CaCl₂, 5 mM KCl, 2 mM MES, pH 5.7) and harvested through 40 μm sterile cell filters into sterile 50 mL conical tubes, then centrifuged at 150×g for 5 minutes. Supernatant was removed and cells were resuspended in 2 mL At-W5 buffer by gently rocking, then slowly layered onto 6 mL of 0.6 M sucrose solution as described in Jeong et al. 2021 (Optimization of protoplast regeneration in the model plant Arabidopsis thaliana. Plant methods 17(1): 1-16) in a new tube. Tubes were centrifuged at 150×g for 8 minutes. After centrifugation a thick dark band of protoplasts appeared at the interface of the two solutions. This band was harvested in one continuous motion using a sterile serological pipette and combined with 30 mL of At-W5 buffer. The samples were centrifuged at 150×g for 5 minutes and resuspended in 10 mL of At-W5 buffer. Cells were quantified using a Bürker hemocytometer and stored at 4° C. in the dark until transfection. A sample was reserved from each isolation to test cell viability using FDA staining as described by Larkin 1976 (Purification and viability determinations of plant protoplasts. Planta. 128(3): 213-216).

### AM2: Transfection

Protoplast transfection proceeded in a similar fashion to Yoo et al. 2007 (Arabidopsis mesophyll protoplasts: a versatile cell system for transient gene expression analysis. Nature protocols. 2(7): 1565-1572). Protoplasts were centrifuged at 150×g for 5 minutes and resuspended in a volume of transformation MMG buffer (4 mM MES, pH 5.7, 0.4 M mannitol and 15 mM MgCl₂) sufficient to achieve a cell density of 2×10⁶ protoplasts/mL. For each transfection reaction, 20 μL of freshly prepared RNP solution as described in Example 1's section GM2 was added to the bottom of a 15 mL round bottom tube, and then 100 μL of protoplasts suspended in transformation MMG buffer was added and mixed with the RNP solution. Next, 120 μL of freshly prepared PEG solution (40% PEG4000, 0.2 M mannitol and 100 mM CaCl₂) was added and gently mixed by rotating the tubes. After a 15-minute incubation at room temperature, the transfection mixture was diluted with 5 mL of At-W5 solution and gently mixed to stop the transfection process. The transfected cells were then washed twice following centrifugation at 150×g for 5 minutes using a 0.5 M mannitol solution. Cells with the same transfection reactions were combined to achieve a final protoplast density of 2×10⁶ protoplasts/mL in 0.5 M mannitol solution for alginate encapsulation.

### AM3: Encapsulation and Regeneration of Plants

An equal volume of transfected cells was combined with a 2.8% sodium alginate solution and then gently mixed together. The protoplast-alginate mixture was then poured onto a CaCl₂ agar plate and incubated at room temperature for 30 minutes, after which 2 mL of CaCl₂ solution was added to further solidify the hydrogel. The protoplast-alginate gel was then cut into 4 pieces and a quarter of the gel was placed in a Petri dish with Protoplast Induction Medium (PIM), as described in Jeong et al. 2021.

Plant regeneration protocols were based on Jeong et al. 2021 with some modifications summarized as follows. Following encapsulation in thin alginate layers, the protoplasts were incubated in darkness for 4 weeks, and fresh PIM was added weekly. Cell division started after about one week, and when microcalli reached a size of about 500 μm, the PIM was replaced with Callus Induction Medium (CIM). Calli were allowed to grow to about 1-2 mm size in CIM, after which they were plated on solid Shoot Induction Medium (SIM). After 3 weeks, or when the shoots reached a size of approximately 5-10 mm, they were excised from the calli and transferred to Rooting Medium (RM).

Plants were then propagated in vitro and genotyped using the same sequencing methods described in Example 1's section GM3. Plants containing bi-allelic mutations in target MiMe genes were further multiplied and prepared for planting.

### AM4: Plant Growth and Crossing Conditions

Once plants had completely regenerated in vitro from protoplasts, rooted plantlets were transferred to 3.5-inch pots filled with wetted PROMIX HP Growing Medium (Premier Tech Horticulture, Quakertown, PA, USA). 18 pots were placed in each tray, and trays were covered with plastic domes for 3 days. Afterwards, lids were slowly removed by opening them more every day over the course of 3 days. Plants were grown under 18-hour light/6-hour dark photoperiod (40-70 μmol photons m-2 s-1) at 21-22° C. Plants were watered with distilled water and fertilized with 1× Miracle Grow, indoor concentration (The Scotts Company LLC, Marysville, OH, USA) dissolved in distilled water.

Regenerated plants with confirmed edits in each target gene including REC8, OSD1, and PAIR1 and/or SPO11 were transplanted from tissue culture into substrate and then acclimatized under cool white lights at 24° C. with approximately 60% relative humidity and 16-hour daylength. When plants produced flowers, pollen was collected, and cross pollination was performed under the same environmental conditions. About 21 days after pollination, siliques were removed and seeds were extracted, dried, and prepared for planting.

### AM5: Seed Germination and Genotyping

Following seed extraction, seeds were surface sterilized using 1 mL of 70% ethanol solution for 5 min. Then, seeds were rinsed with 1 mL of sterile, distilled water five times. Sterilized seeds were sown on hormone-free AGM (Arabidopsis Germination Medium) in Petri dishes. After one week, seedlings exhibiting germination were transplanted to soil, where they were allowed to grow for 2 months before leaves were removed for DNA extraction.

High quality genomic DNA was extracted from leaves of randomly selected plants from each population used for genotyping using Qiagen Dneasy Plant Mini kits (Cat. No. 69104) (QIAGEN, Germantown, MD, USA). Purity was determined using a spectrophotometer and DNA was quantified with a fluorometer as described in Example 2's section PM5. Samples were adjusted to a final concentration of 20 ng/μL. Markers were established and populations were genotyped as described in GM6.

### AM6: Fertility and Parthenocarpy Testing

Fertility was determined by counting the number of seeds per fruit (silique) for 20 unopened, mature fruits per plant. Parthenocarpic fruit development was induced by application of gibberellic acid. Pistils were treated with a 0.1 μmol/μL gibberellic acid solution (“GA3 treated”) or with a buffered control (“mock-treated”) solution as described in Vivian-Smith et al. 1999 (Genetic analysis of growth-regulator-induced parthenocarpy in Arabidopsis. Plant Physiology. 121(2): 437-452). Flowers were not emasculated prior to treatment. Some flowers were left to develop normally (“untreated”). On 8 days post-application, fruit length was measured, and on 12 days post-application, some fruits were removed for clearing according to the protocol outlined in Sprunck et al. 2012 (Egg cell-secreted EC1 triggers sperm cell activation during double fertilization. Science. 338(6110): 1093-1097). Cleared fruits were imaged the following day.

### AR: Results

### AR1: Guide RNA Screening

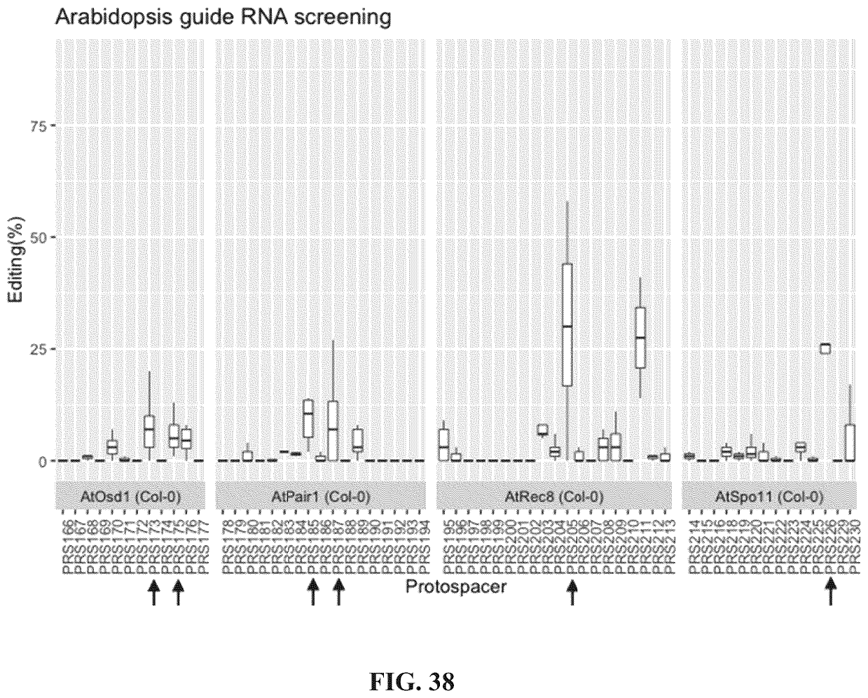

A total of 62 guide RNAs (Table 16; SEQ ID NOs: 324-385) targeting the conserved regions of the OSD1, SPO11-1, REC8 or PAIR1 genes provided by Table 17's SEQ ID NOs: 386-447 were transfected in triplicate in protoplasts from multiple plants from the Arabidopsis accessions described in section AM1. Editing efficiency was calculated by sequencing as described in Example 1's section GM3, and the resulting data presented in FIG. 38. Based on this data, guide RNAs SEQ ID NO: 331 and SEQ ID NO: 333 were chosen for editing the OSD1 gene; SEQ ID NO: 343 and SEQ ID NO: 345 were chosen for editing the PAIR1 gene; SEQ ID NO: 363 was chosen for editing the REC8 gene; and SEQ ID NO: 383 was chosen for editing the SPO11-1 gene.

TABLE 16
Sequences for guide RNAs
targeting MiMe genes in A. thaliana.

| SEQ ID NO. | PRSG# | Guide RNA sequence |
|---|---|---|
| 324 | PRS166G | UUUCAGCGACGUUAUCAAAA |
| 325 | PRS167G | AGCGACGUUAUCAAAAUGCC |
| 326 | PRS168G | CCAACCGACGGAGACAUGGU |
| 327 | PRS169G | ACUUGACGAGCCAGAUUCAC |
| 328 | PRS170G | CUUGACGAGCCAGAUUCACG |
| 329 | PRS171G | AUUGAAUCUCCGGUGAAUCC |
| 330 | PRS172G | ACUACAUGGAGGCCUGGUAA |
| 331 | PRS173G | GAUUGCCACAGGGAAGAGAG |
| 332 | PRS174G | UUGCCUUCUUGGUAUCCAAG |
| 333 | PRS175G | UGUUCUCCUGACAGUGGUAC |
| 334 | PRS176G | GCAGUUGAUGGUGGGCAACU |
| 335 | PRS177G | AACUUUAGCAGUUGAUGGUG |
| 336 | PRS178G | UUAAUAUUCAUCUUCAUCAC |
| 337 | PRS179G | UGUCUUUCCUCCUAAUUUAA |
| 338 | PRS180G | GAAUUAGAAGGAGAUCAGCA |
| 339 | PRS181G | CUCAGAUGACUCAGAGUUCU |
| 340 | PRS182G | CCUUUUUGCACAGAGAUUUA |
| 341 | PRS183G | UGCACAGAGAUUUAGUUCUC |
| 342 | PRS184G | CUGAGAACUAAAUCUCUGUG |
| 343 | PRS185G | GUUCUCAGGAAAGAGAUCUC |
| 344 | PRS186G | AAGAAGGUUAGUAGUUGCUU |
| 345 | PRS187G | UGAUUGAUUGGAGGUAAGCA |
| 346 | PRS188G | CUUCAUUUGUCACCUAGUAG |
| 347 | PRS189G | GGAUGAUGGAGACUUCUUUG |
| 348 | PRS190G | AGCAGGUUUGGAAUGAUGUU |
| 349 | PRS191G | GUUCCUCGAUUGGCUUGCAU |
| 350 | PRS192G | AAGAAAUACUUCUUUAGUUC |
| 351 | PRS193G | UUCUGUUUACUGCAGCUGAA |
| 352 | PRS194G | GAAUCGGCUUGUUCCUUUCU |
| 353 | PRS195G | UUCUCACCAGCUUCUAGCUC |
| 354 | PRS196G | ACUCACCAUAUCUGACCCAA |
| 355 | PRS197G | ACUGAUAGAAUUUGACUCAC |
| 356 | PRS198G | UUCCGGUUGAUCUUCGCGUG |
| 357 | PRS199G | AAUGAUAUCGAGCUUAUCUA |
| 358 | PRS200G | CUCUUACAGCGAAGAGAUUU |
| 359 | PRS201G | GAAUCCGUCGGUUCCGAUGG |
| 360 | PRS202G | GUUUAUUCGAUAGGUGGUGU |
| 361 | PRS203G | UUCGAUAGGUGGUGUUGUGA |
| 362 | PRS204G | CUCUCAUAAACAAUCACAAC |
| 363 | PRS205G | UGAGAGGAAAGUGAAGCUCC |
| 364 | PRS206G | AAUCAGAAAAUUACCGAAUA |
| 365 | PRS207G | CGUCAAUAUUUCAGAUGAUG |
| 366 | PRS208G | AGAUGAUGUUAAUCGCUUUC |
| 367 | PRS209G | AUUACCAGAAAGCGAUUAAC |
| 368 | PRS210G | UCUCAGGUUGAAAUUAAUGG |
| 369 | PRS211G | UCUCAAGAUACUCACCUUGA |
| 370 | PRS212G | UGUAACAUUCUCAGCUGAUG |
| 371 | PRS213G | AUCAUCUCCUUCGAUGUCAA |
| 372 | PRS214G | AGAAUCGACUAAUUUGCUUC |
| 373 | PRS215G | AAGCAAAUUAGUCGAUUCUG |
| 374 | PRS216G | AUCCUUUGAAGCAAAUUAGU |
| 375 | PRS218G | GAGCAGAUUUUACUCAAUCG |
| 376 | PRS219G | CUCAAUCGGUCGUUGUAGAU |
| 377 | PRS220G | GAGAUCUUCCUUCAGCCAGA |
| 378 | PRS221G | CAUCAAUCAAUUCAGGAACU |
| 379 | PRS222G | AUUCUGCUGUCUAUAGCUUA |
| 380 | PRS223G | GUUUAUCAGAGCUGCAUAAG |
| 381 | PRS224G | CUGACCCUUUGGUUUAUCAG |
| 382 | PRS225G | CACUCUUAAGAAGGAACCAC |
| 383 | PRS226G | UGGUUCCUUCUUAAGAGUGA |
| 384 | PRS229G | CGAAGCAUGUCUAUUUUCUU |
| 385 | PRS230G | ACCAACCCAUCACCAACCUA |

TABLE 17
Sequences for conserved regions
of MiMe genes targeted in A. thaliana.

| SEQ ID NO. | PRS# | Target DNA sequence |
|---|---|---|
| 386 | PRS166 | TTTCAGCGACGTTATCAAAA |
| 387 | PRS167 | AGCGACGTTATCAAAATGCC |
| 388 | PRS168 | CCAACCGACGGAGACATGGT |
| 389 | PRS169 | ACTTGACGAGCCAGATTCAC |
| 390 | PRS170 | CTTGACGAGCCAGATTCACG |
| 391 | PRS171 | ATTGAATCTCCGGTGAATCC |
| 392 | PRS172 | ACTACATGGAGGCCTGGTAA |
| 393 | PRS173 | GATTGCCACAGGGAAGAGAG |
| 394 | PRS174 | TTGCCTTCTTGGTATCCAAG |
| 395 | PRS175 | TGTTCTCCTGACAGTGGTAC |
| 396 | PRS176 | GCAGTTGATGGTGGGCAACT |
| 397 | PRS177 | AACTTTAGCAGTTGATGGTG |
| 398 | PRS178 | TTAATATTCATCTTCATCAC |
| 399 | PRS179 | TGTCTTTCCTCCTAATTTAA |
| 400 | PRS180 | GAATTAGAAGGAGATCAGCA |
| 401 | PRS181 | CTCAGATGACTCAGAGTTCT |
| 402 | PRS182 | CCTTTTTGCACAGAGATTTA |
| 403 | PRS183 | TGCACAGAGATTTAGTTCTC |
| 404 | PRS184 | CTGAGAACTAAATCTCTGTG |
| 405 | PRS185 | GTTCTCAGGAAAGAGATCTC |
| 406 | PRS186 | AAGAAGGTTAGTAGTTGCTT |
| 407 | PRS187 | TGATTGATTGGAGGTAAGCA |
| 408 | PRS188 | CTTCATTTGTCACCTAGTAG |
| 409 | PRS189 | GGATGATGGAGACTTCTTTG |
| 410 | PRS190 | AGCAGGTTTGGAATGATGTT |
| 411 | PRS191 | GTTCCTCGATTGGCTTGCAT |
| 412 | PRS192 | AAGAAATACTTCTTTAGTTC |
| 413 | PRS193 | TTCTGTTTACTGCAGCTGAA |
| 414 | PRS194 | GAATCGGCTTGTTCCTTTCT |
| 415 | PRS195 | TTCTCACCAGCTTCTAGCTC |
| 416 | PRS196 | ACTCACCATATCTGACCCAA |
| 417 | PRS197 | ACTGATAGAATTTGACTCAC |
| 418 | PRS198 | TTCCGGTTGATCTTCGCGTG |
| 419 | PRS199 | AATGATATCGAGCTTATCTA |
| 420 | PRS200 | CTCTTACAGCGAAGAGATTT |
| 421 | PRS201 | GAATCCGTCGGTTCCGATGG |
| 422 | PRS202 | GTTTATTCGATAGGTGGTGT |
| 423 | PRS203 | TTCGATAGGTGGTGTTGTGA |
| 424 | PRS204 | CTCTCATAAACAATCACAAC |
| 425 | PRS205 | TGAGAGGAAAGTGAAGCTCC |
| 426 | PRS206 | AATCAGAAAATTACCGAATA |
| 427 | PRS207 | CGTCAATATTTCAGATGATG |
| 428 | PRS208 | AGATGATGTTAATCGCTTTC |
| 429 | PRS209 | ATTACCAGAAAGCGATTAAC |
| 430 | PRS210 | TCTCAGGTTGAAATTAATGG |
| 431 | PRS211 | TCTCAAGATACTCACCTTGA |
| 432 | PRS212 | TGTAACATTCTCAGCTGATG |
| 433 | PRS213 | ATCATCTCCTTCGATGTCAA |
| 434 | PRS214 | AGAATCGACTAATTTGCTTC |
| 435 | PRS215 | AAGCAAATTAGTCGATTCTG |
| 436 | PRS216 | ATCCTTTGAAGCAAATTAGT |
| 437 | PRS218 | GAGCAGATTTTACTCAATCG |
| 438 | PRS219 | CTCAATCGGTCGTTGTAGAT |
| 439 | PRS220 | GAGATCTTCCTTCAGCCAGA |
| 440 | PRS221 | CATCAATCAATTCAGGAACT |
| 441 | PRS222 | ATTCTGCTGTCTATAGCTTA |
| 442 | PRS223 | GTTTATCAGAGCTGCATAAG |
| 443 | PRS224 | CTGACCCTTTGGTTTATCAG |
| 444 | PRS225 | CACTCTTAAGAAGGAACCAC |
| 445 | PRS226 | TGGTTCCTTCTTAAGAGTGA |
| 446 | PRS229 | CGAAGCATGTCTATTTTCTT |
| 447 | PRS230 | ACCAACCCATCACCAACCTA |

### AR2: Generation of Parent MiMe Plants

Regenerated plants were screened using the sequencing methods described in Example 1's section GM3. About 1% of the regenerated plants screened (1,903 total plants) had mutations predicted to result in either nonfunctional or non-expressed MiMe alleles, resulting in a MiMe genotype. Such plants were chosen for the crosses described below, which had mutations predicted to result in either nonfunctional or non-expressed rec8, osd1, and pair1 alleles, or nonfunctional or non-expressed rec8, osd1, and spo11-1 alleles.

Two unique plants designated PED-AR-BC and PED-AR-AA were chosen to move forward as parent MiMe plants for partial complementation crosses, within which each parent which had bi-allelic edits resulting in two distinctive complete MiMe genotypes for rec8, osd1, pair1, and rec8, osd1, spo11-1, respectively. Microscopic analysis of microsporogenesis showed these parent MiMe plants also produced dyads instead of tetrads, and also produced viable pollen. The parent MiMe plant PED-AR-BC (rec8/osd1/pair1) was derived from F2 individuals which themselves were derived from a selfed F1 hybrid between Col-0 (CS851557-S1) and mdiploid inbred HR-10. The parent MiMe plant PED-AR-AA (rec8/osd1/spo11-1) was derived from an inbred individual of Shahdara.

Two unique plants designated PED-AR-BC and PED-AR-DE were chosen to move forward as parent MiMe plants for crossing, each of which had bi-allelic edits resulting in a complete MiMe genotype for rec8, osd1 and pair1. Microscopic analysis of microsporogenesis showed that all three parent MiMe plants produced dyads instead of tetrads and produced viable pollen. The parent MiMe plant PED-AR-DE (rec8/osd1/pair1) was derived from an F2 plant which itself derived from a selfed F1 hybrid between diploid inbred Col-0 and diploid inbred HR-10.

As an example, edits for the parent MiMe plant PED-AR-AA (rec8/osd1/spo11-1) are illustrated in FIGS. 39A-39C. In that figure, the predicted protein sequences are illustrated with respect to the unedited wildtype protein sequence and a consensus sequence, wherein an “X” in the consensus sequence indicates that there is no consensus for that amino acid position; wherein a dot (“.”) in the parent MiMe plant sequence indicates that the respective amino acid matches the consensus sequence; and wherein a dash (“-”) in the parent MiMe plant sequence indicates that the respective amino acid is present in the consensus sequence but is absent in the parent MiMe plant sample sequence. Frameshift inducing deletions in alleles of osd1 (FIGS. 39A, 39D, and 39E), spo11-1 (FIGS. 39B and 39F), one allele of rec8 (FIGS. 39C and 39G), and pair1 (FIG. 39H) result in premature stop codons. Premature stop codons are denoted by an asterisk (“*”) in FIGS. 39A-39C. Another allele of rec8 has a 9 bp deletion resulting in the loss of three (3) amino acid residues in a highly conserved domain of the protein which is expected to disrupt its function (FIGS. 39C and 39G).

### AR3: Generation of Genetically Uniform Tetraploid Arabidopsis Seed

Parent MiMe plants were crossed using hand pollination. Seeds resulting from crosses were germinated following the procedures described in section AM1. In total, two genetically uniform populations of tetraploid Arabidopsis seed with either three or four haplotypes were created, from the parent MiMe plants described in section AR2.

The first genetically uniform population of tetraploid Arabidopsis seed referred to herein as Boosted Arabidopsis Population 1 (“BAP1”) consisted tetraploid individuals with three haplotypes, derived from a cross between a female parent MiMe plant PED-AR-BC (rec8/osd1/pair1) and a male parent MiMe plant PED-AR-AA (rec8/osd1/spo11-1).

The second genetically uniform population of tetraploid Arabidopsis seed referred to herein as Boosted Arabidopsis Population 2 (“BAP2”) consisted of tetraploid individuals with four haplotypes derived from a cross between a female parent MiMe plant PED-AR-BC (rec8/osd1/pair1) and a male parent MiMe plant PED-AR-DE (rec8/osd1/pair1).

### AR4: Flow Cytometry

For each individual, young leaf tissue or inflorescences were chosen for flow cytometry. All MiMe parent plants used for crosses were shown to be diploid having mean peak fluorescences consistent with diploid control plants. All individuals from BAP1 and BAP2 (except 1 individual from BAP1) were shown to be tetraploid with mean peak fluorescence values double that of diploid control plants. One individual from BAP1 had an intermediate mean peak between the tetraploid and diploid peaks, indicative of triploidy.

### AR5: Genotypic Evaluation of BAP1, BAP2, and MiMe Parent Plants

Reads for wild type HR-10 were obtained from the Sequence Read Archive SRA (SRR492282). DNA samples were sequenced by Novogene. Libraries were prepared using the NEBNext® Ultra™ II for DNA Library Prep kit, and paired-end 2×150 reads were sequenced on an Illumina NovaSeq 6000 or NovaSeq X plus.

For the parent MiMe plants PED-AR-BC and PED-AR-AA, DNA samples were sequenced to a depth of 6,261,903,000 bp, and 6,856,028,700 bp, respectively. For the BAP1 progeny plants, DNA samples were sequenced to an average depth of 3,735,085,029 bp. For the parent MiMe plant PED-AR-DE, DNA samples were sequenced to a depth of 4,690,109,700 bp. For the BAP2 progeny plants, DNA samples were sequenced to an average depth of 3,938,238,717 bp. Reads were aligned to the TAIR10 reference genome with BWA-MEM (v0.7.17-r1188) (Li, H. 2013) and alignments were sorted and indexed with samtools (v1.15.1).

For BAP1, variants were called and jointly genotyped for parents (PED-AR-AA and PED-AR-BC), wild type HR-10, and all 100 progeny plants with GATK (v4.3.0.0) (McKenna, A et al. (2010) The Genome Analysis Toolkit: a MapReduce framework for analyzing next-generation DNA sequencing data. Genome research, 20(9), pp. 1297-1303). To identify heterozygous blocks in the F2 parent, only SNPs with the following characteristics are considered for analysis: (i) the SNP is either bi-allelic or tri-allelic amongst the three parental haplotypes; (ii) the SNP is homozygous ALT/ALT in HR-10 and not missing in PED-AR-BC; (iii) the SNP has a Mapping Quality (MQ) score of 60 and it is covered by at least 20 reads in PED-AR-BC; and (iv) the SNP is not within 200 bp of any indel. Using these SNPs, larger genotype blocks were defined by binning SNPs into non-overlapping 100 kbp windows. Each bin was assigned the modal genotype of its constituent SNPs. This method partitioned the PED-AR-BC genome into blocks of either homozygous REF/REF (0/0), homozygous ALT/ALT (1/1) or heterozygous REF/ALT (0/1) states. In total, six heterozygous blocks were identified spanning 61,735,056 bp (51.81% of the TAIR10 reference genome). A similar method was used to identify heterozygous blocks in PED-AR-DE. After establishing heterozygous blocks in the hybrid parents, markers were established and populations were genotyped as described in GM6.

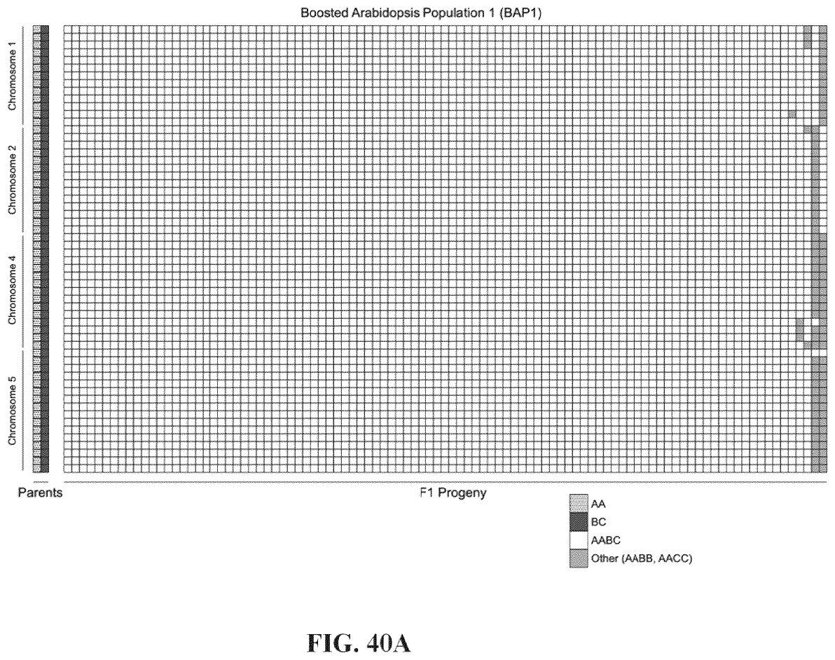

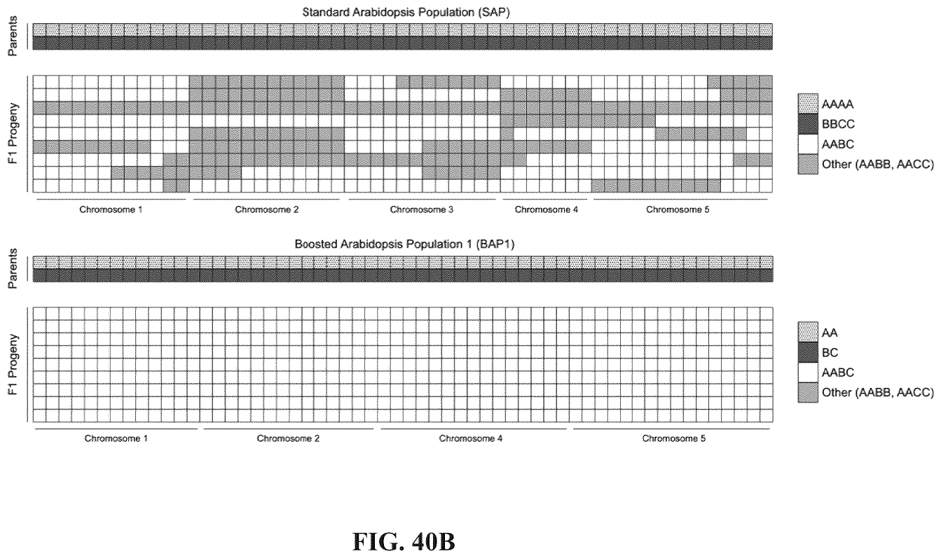

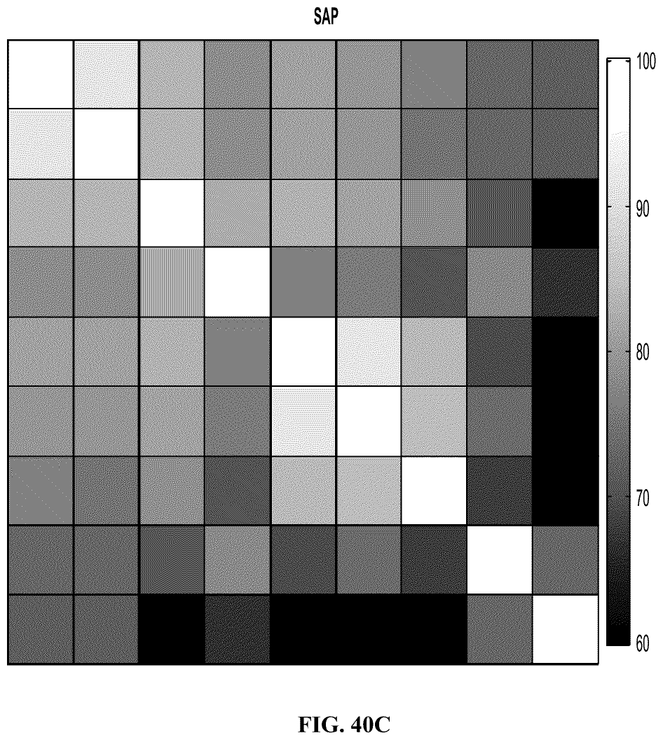

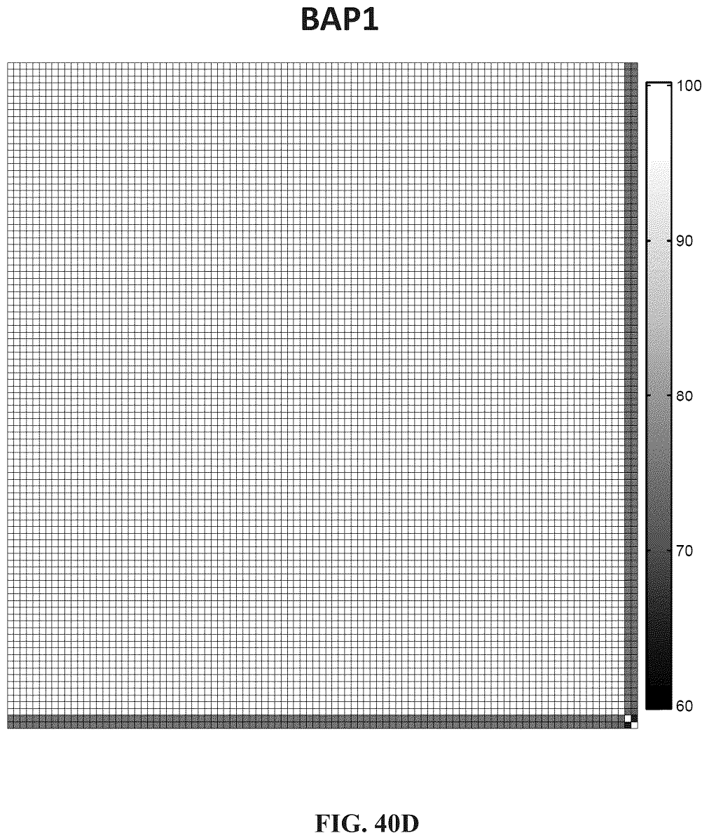

After filtering for high quality markers and excluding the one triploid plant, a genotype matrix was assembled for BAP1 (FIG. 40A). Across the BAP1 population, the average pairwise genetic uniformity as measured by the Jaccard similarity coefficient was 98.9% (visualized as a pairwise similarity matrix in FIG. 40D). These data are illustrated by the population uniformity graph in FIG. 35. The genetic uniformity was similar for the BAP2 population.

### AR6: Genotypic Evaluation of Non-MiMe Progeny (SAP)

The extent of genotypic uniformity of a standard population of non-MiMe x non-MiMe tetraploid Arabidopsis seed was also determined. The population of non-MiMe tetraploid Arabidopsis seed referred to herein as the Standard Arabidopsis Population (“SAP”) consisted of nine tetraploid individuals derived from a cross between a female parent non-MiMe tetraploid plant Col-0 (CS851557-S1) x HR-10 (doubled F2 hybrid obtained through protoplast regeneration) and a male parent non-MiMe tetraploid plant Shahdara (doubled inbred obtained through protoplast regeneration).

Reads for wild type HR-10 were obtained from the Sequence Read Archive SRA (SRR492282). DNA samples were sequenced by Novogene. Libraries were prepared using the NEBNext® Ultra™ II for DNA Library Prep kit, and paired-end 2×150 reads were sequenced on an Illumina NovaSeq 6000. For the female parent non-MiMe plant (Col-0 (CS851557-S1) x HR-10 F2), DNA samples were sequenced to a depth of 5,956,964,100 bp. For SAP plants, DNA samples were sequenced to an average depth of 3,998,928,200 bp. Markers were established and populations were genotyped as described in GM6.

After filtering for high quality markers, a genotype matrix was assembled for SAP (FIG. 40B). Across the SAP population, the average pairwise genetic uniformity as measured by the Jaccard similarity coefficient was 75.2% (visualized as a pairwise similarity matrix in FIG. 40C). These data are further illustrated by the population uniformity graph in FIG. 35.

### AR7: Phenotypic Evaluation, Parthenocarpy, and Enlarged Seedless Fruit

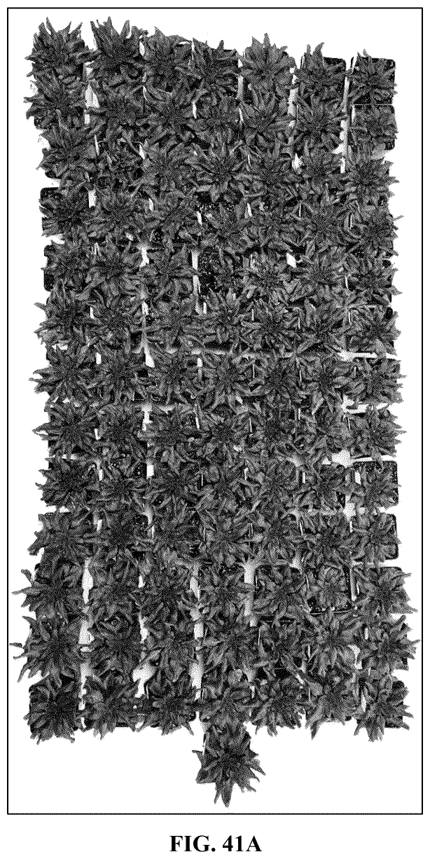

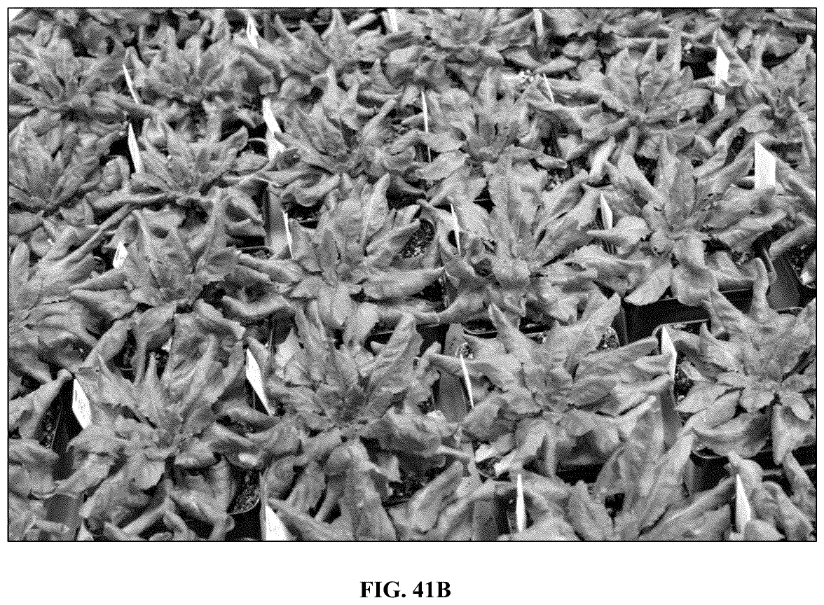

The plants from BAP1 and BAP2 were uniform in size, growth and appearance for their respective population. For example, plants from BAP1 are shown in FIG. 41A at 48 days after planting. A subset of the same plants from BAP1 are shown from a closer distance in FIG. 41B, also at 48 days after planting.

Plants from BAP1 had a partially-complemented spo11-1/pair1 Mime genotype (rec8/rec8, osd1/osd1, spo11-1/pair1) which did not exhibit a wild-type meiosis phenotype because the MiMe loci of the first MiMe component and the MiMe loci of the third MiMe component of both the first and second parent MiMe plants were rec8 and osd1 respectively at both loci, but which also did not exhibit a MiMe phenotype either due to the complementation of a first MiMe locus (pair1) of the second MiMe component with a second MiMe locus (spo11-1) of the second MiMe component. Instead, plants from BAP1 produced parthenocarpic seedless fruit (siliques)

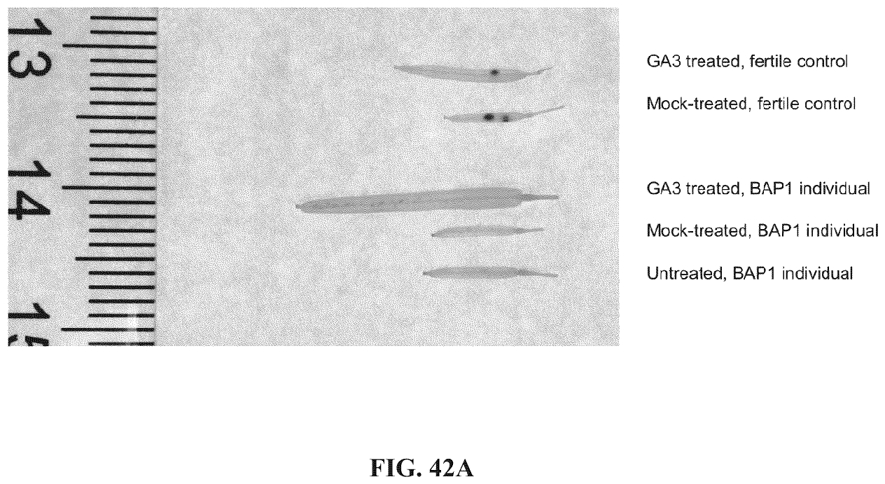

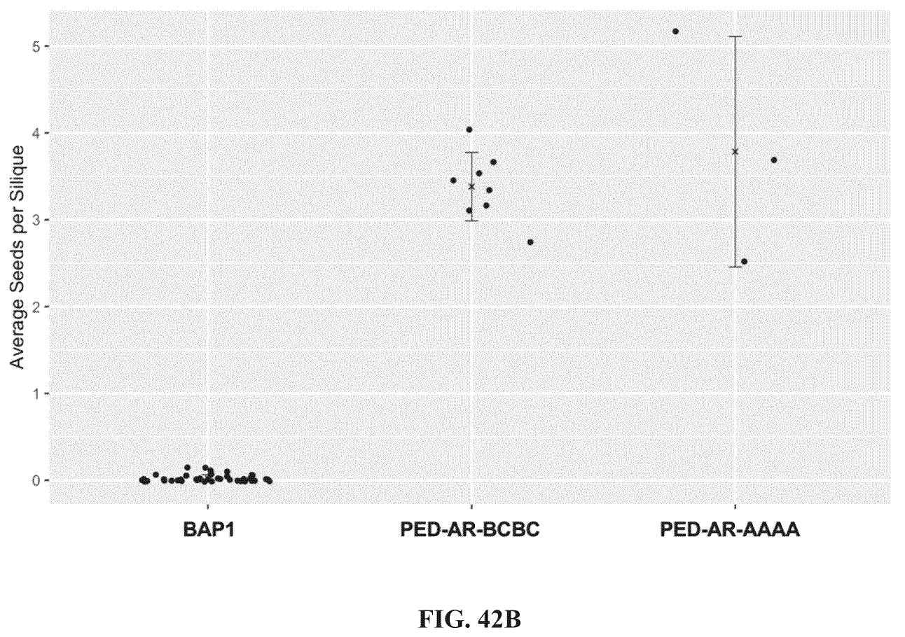

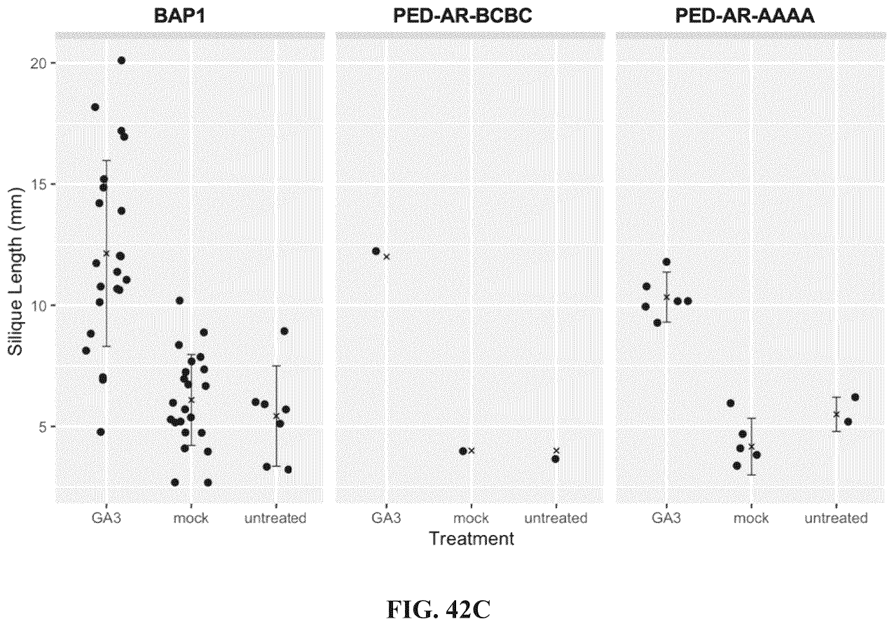

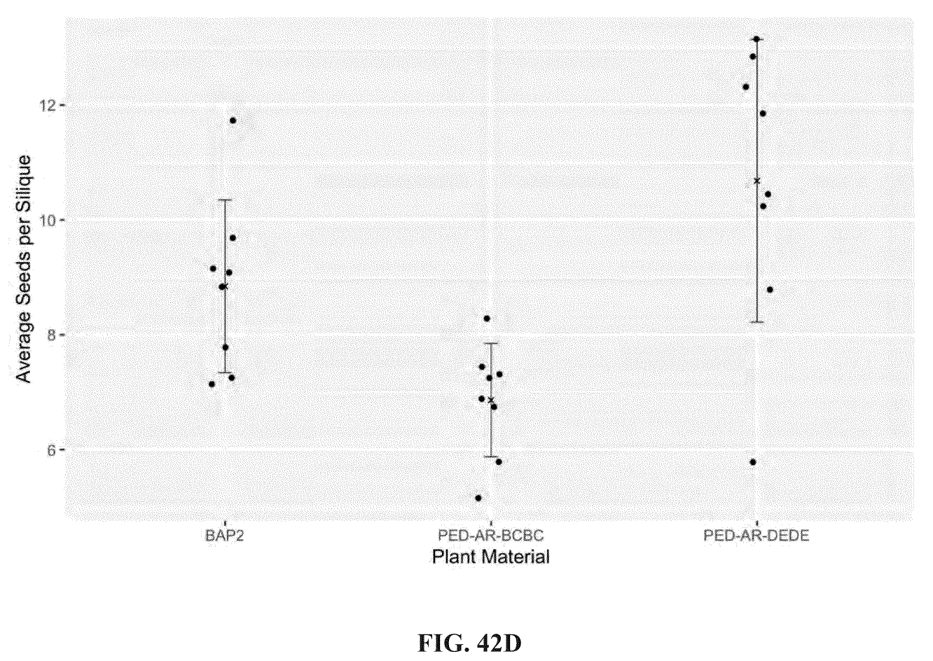

For example, FIG. 42A shows seedless fruit from BAP1 plants treated, untreated or mock-treated with gibberellic acid (GA3) to induce fruit development, compared to fruit from treated or mock-treated fertile Arabidopsis control plants as described in section AM6. When 42 plants from BAP1 were randomly selected and 20 fruits were randomly observed from each selected plant, it was determined that 31 of the 42 BAP1 plants produced no seeds, and the average amount was 0.15 seeds/fruit when considering all 42 plants. The seeds that were collected from the BAP1 plants failed to germinate under standard conditions, and demonstrating that all 42 plants from BAP1 failed to produce viable seed, and can therefore be considered seedless. In contrast, the fertile Arabidopsis control plants produced about 3 to 4 seeds/fruit. (FIG. 42B). The control plants were doubled versions of the BAP1 parent MiMe plants designated as PED-AR-AAAA (doubled version of PED-AR-AA) and PED-AR-BCBC (doubled version of PED-AR-BC).

As illustrated in FIG. 42A, the size of the fruit from GA3-treated BAP1 plants was often enlarged compared to fruit from control plants. The increase in fruit length (mm) was quantified for GA3-treated BAP1 compared to those from controls and illustrated in the graph provided by FIG. 42C. As illustrated in the photograph in FIG. 42A, the thickness and overall size of BAP1 fruit was also enlarged compared to that from a control plant.

On the other hand, plants from BAP2 were fertile with an average of 8.84 seeds/fruit as illustrated in FIG. 42D. The control plants were doubled versions of the BAP2 parent MiMe plants designated as PED-AR-BCBC (doubled version of PED-AR-BC) and PED-AR-DEDE (doubled version of PED-AR-DE). PED-AR-BCBC plants had an average of 6.86 seeds/fruit, whereas PED-AR-DEDE plants had an average of 10.68 seeds/fruit.

Without being held to any one particular theory, these results can be interpreted to indicate that functional copies of SPO11-1 and PAIR1 complement their respective knockouts and restore the cellular function of DNA double strand breakage, thus having essentially the same phenotype as a rec8 mutant and producing fragmented chromosomes at the end of the first meiotic division. The skipping of a second division becomes irrelevant because the fragmented chromosomes will not produce viable gametes. Because viable gametes are not produced, egg cells cannot be produced to lead to seeds. Similarly, complementing cyca1 and osd1 will result in similar phenotype due to progression through the second division of meiosis causing random or non-assortment of chromosomes.

### Example 8: Breeding to Combine Edited and/or Natural MiMe Alleles in Grandparent Non-MiMe Plants to Produce Parent MiMe Plants

This Example describes a method of producing a population of polyploid seed by first generating four grandparent non-MiMe plants having partial MiMe genotypes comprising edited and/or natural MiMe alleles, then crossing the grandparent non-MiMe plants to produce two parent MiMe plants having complete MiMe genotypes, and finally crossing the two parent MiMe plants to produce the population of polyploid seeds. Exemplary variations of this method are depicted in FIGS. 18 and 19. This method may be used to produce polyploid hybrid seeds having three or more haplotypes, e.g., if the grandparent non-MiMe plants together comprise three or more haplotypes. The methods are described below for maize, but may be modified to be applied to any polyploid plant for which suitable transformation and gene editing methods are known to one of skill in the art.

A construct for expressing a nuclease (e.g., pMEM4 shown in FIG. 12, or a similar construct) is bombarded into maize line PED495, and events are selected as described in Wang and Frame (2009. Biolistic gun-mediated maize genetic transformation. Transgenic maize: methods and protocols, pp. 29-45). Separately, a construct for expressing a gRNA targeting a MiMe locus (e.g., pMEM6b as shown in FIG. 13 or a similar construct) is bombarded into maize line PED495, and events are selected as described in Wang and Frame (2009). T₀ plants containing the nuclease construct are crossed with T₀ plants containing the gRNA construct to produce T₁F₁ hybrid plants containing both transgenes. The T₁F₁ hybrid plants are screened for editing of the MiMe locus targeted by the gRNA, and any plants with editing are self-pollinated or outcrossed to produce plants heterozygous for knock-out edits of individual MiMe loci. Plants that are heterozygous for edits of single or multiple MiMe loci are crossed together or crossed to natural knockout alleles to produce four plants, each heterozygous for the targeted MiMe genotype, e.g., osd1/- (e.g., osd1-1/-, osd1-2/-, osd1-3/-, or any combination thereof), spo11/-, and spo8/- (i.e., each having a partial MiMe genotype). These plants are called A_{HetMiMe}, B_{HetMiMe}, C_{HetMiMe}, and D_{HetMiMe} and are considered grandparent non-MiMe plants each having a partial MiMe genotype. These plants are crossed in an AxB and CxD fashion and screened for plants with a homozygous knockout genotype at all 4 MiMe alleles. These plants are called AB_{MiMe} and CD_{MiMe}. These are considered parent MiMe plants and are recovered at a rate of approximately 1/256. These AB_{MiMe} and CD_{MiMe} parent MiMe plants can then be crossed together to produce a population tetraploid ABCD seed which can be grown into a tetraploid ABCD plant.

### Example 9: Editing of Hybrid Plant Lines to Directly Produce Parent MiMe Plants

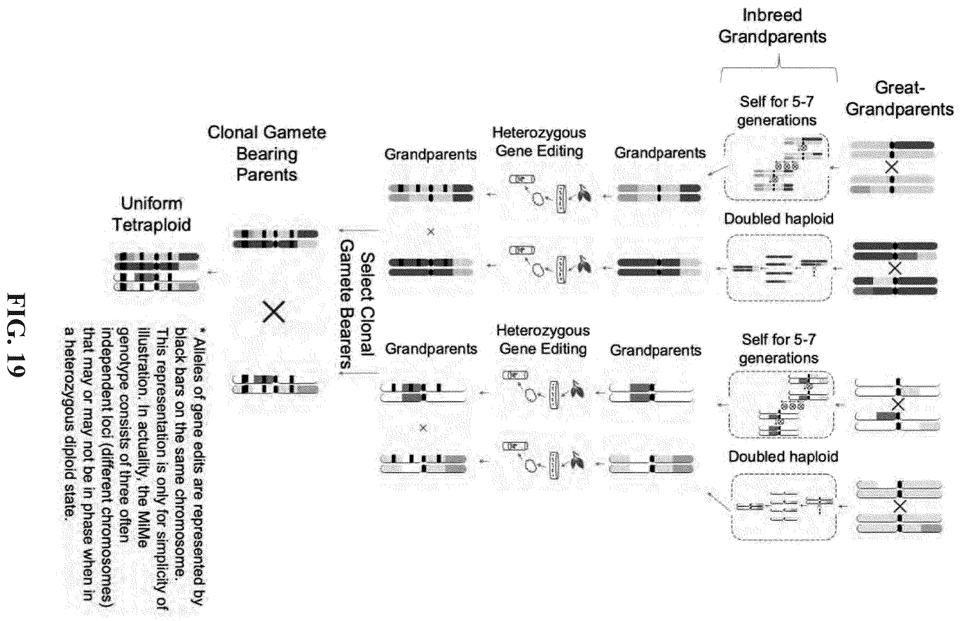

This Example describes an additional method to produce parent MiMe plants by directly editing two hybrid plant lines and screening for plants with a complete MiMe genotype. Exemplary methods of breeding and producing hybrid polyploid seed comprising directly editing two hybrid plant lines to produce parent MiMe plants are depicted in FIGS. 16, 17, and 23. This method may be used to produce polyploid hybrid seeds having three or more haplotypes, e.g., if two hybrid lines together comprising three or more haplotypes are directly edited to produce the parent MiMe plants. The methods are described below for maize, but may be modified to be applied to any plant for which suitable transformation methods are known to one of skill in the art.

PED071 and hybrid PED495 are crossed together to make hybrid embryos that are co-bombarded with gRNA construct pOGZ2 (FIG. 14), and one of pMEM4 (FIG. 12), or pOGZ4 (FIG. 15) as a nuclease construct as well as a selectable marker for bialaphos resistance as described in Wang and Frame (2009) with modifications (using described bombardment with TransIT®-2020). Additionally, a hybrid of PED071 and PED195 is transformed with an identical co-bombardment and transgenic plants can be selected per McCaw et al. (2020. Development of a Transformable Fast-Flowering Mini-Maize as a Tool for Maize Gene Editing. Frontiers in Genome Editing. Doi: 10.3389/fgeed.2020.622227), with modifications of using bombardment as described (using described bombardment with TransIT®-2020) rather than transformation with Agrobacterium. The hybrid transformants are screened for two or more independent events with complete knockout MiMe genotype to act as parent MiMe plants. These parent MiMe plants are crossed to produce a uniform population of tetraploid seed having 3 or more haplotypes, which may be grown into a plant having 3 or more haplotypes. These 3 or more haplotypes may be a combination of PED071, PED195, and an F2 segregant of PED495. They may also be a combination of PED071 and two distinct genotypes that are the result of F2 segregation of PED495.

### Example 10: Additional Methods of Producing Tetraploid Potatoes with Three Haplotypes

### 3.1 Homozygous Tetraploid Plant x Heterozygous Diploid Parent MiMe Plant

In this method, a diploid hybrid (heterozygous) potato plant having two haplotypes is edited to generate a parent MiMe potato plant as described in Examples 1 and 2. The parent MiMe plant may be generated via direct editing of the diploid hybrid potato plant, or may include crossing and selecting for naturally-occurring MiMe alleles, or may include crossing, of two lines with partial MiMe genotypes, where the resulting offspring have a complete MiMe genotype. The parent MiMe plant is confirmed complete MiMe genotype as described herein, such as any of the MiMe genotypes described in Table 10.

Regenerated plants are screened using the NGS sequencing methods described in Examples 1 and 2 and approximately one percent of plants have mutations predicted to result in non-expressed or nonfunctional proteins or other gene products of all of the genes targeted and therefore having the target MiMe genotype, such a one of the MiMe genotypes described in Table 10. Out of between about 2,000 and about 10,000 regenerated plants approximately 20 to 100 unique plants are selected for evaluation in a partial diallel crossing scheme.

The diploid parent MiMe plant is then crossed with a homozygous tetraploid non-MiMe potato plant, lacking the MiMe alleles present in the parent MiMe plant, and having a third haplotype distinct from those in the hybrid parent MiMe plant. Crossing of the reduced (diploid) gametes of the homozygous tetraploid potato plant with the unreduced, clonal (diploid) gametes of the parent MiMe plant results in a uniform population of tetraploid seed having the three haplotypes of the parent plants, as described below.

Approximately 20 hand pollinations are performed per cross resulting in roughly 200 seeds each. Seeds of ten crosses are germinated in vitro after surface disinfesting with 10% bleach and rinsing seeds with sterile deionized water. The average germination rate per cross is between about 50% and about 95% after a period of dormancy. These crosses are evaluated further via chloroplast counts within guard cells using a light microscope to assess Lugol's iodine-stained epidermal leaf peels (diploid=6-8 chloroplasts per guard cell, tetraploid=12-14 chloroplasts per guard cell). One cross of parent plants, with a germination rate of between about 50% and about 95% and chloroplast counts suggestive of tetraploidy, is selected for further evaluation with flow cytometry.

Using the flow cytometry methods described in Examples 1 and 2, the parent MiMe plant is shown to be diploid having DNA content of approximately 1.6-2 pg per 2C nucleus relative to internal controls, and the parent non-MiMe plant is shown to be tetraploid with DNA content of approximately 3.2-4 pg per 2C nucleus. Greater than 50% of the 100 progeny of the cross are shown to be tetraploid with DNA content of approximately 3.2-4 pg per 2C nucleus and show similar variability between family members as observed in technical replicates from the same plant.

Genotypic matrices and haplotyping results show specific loci with three alleles in more than 70% of tetraploid members and also show extended haplotype blocks with three haplotypes. After filtering for high quality markers, the genotyping matrices for the 100 progeny of the tetraploid are greater than 70% similar.

### 3.2 Homozygous Diploid Plant Producing Unreduced Gametes x Diploid Hybrid Parent MiMe Plant

In this method, a diploid hybrid (heterozygous) potato plant having two haplotypes is edited to generate a parent MiMe potato plant, as described in Example 10's section 3.1, which is confirmed complete MiMe genotype as described herein, such as any of the MiMe genotypes described in Table 10. Approximately one percent of plants have mutations predicted to result in non-expressed or nonfunctional proteins or other gene products of all of the genes targeted and therefore having the target MiMe genotype. Out of between 2,000 and 10,000 regenerated plants approximately 20 to 100 unique plants are selected for evaluation in a partial diallel crossing scheme.

Separately, a homozygous diploid potato plant is obtained that has a third haplotype and at least one allele that results in production of unreduced (2n, diploid) gametes. The at least one allele can be naturally occurring allele such as, for example, an os allele. (J. E. Werner & S. J. Peloquin, Inheritance and Two Mechanisms of 2n Egg Fromation in 2× Potatoes, Journal of Heredity, Volume 81, Issue 5, September 1990, Pages 371-374; J. E. Werner & S. J. Peloquin, Occurrence and mechanisms of 2n egg formation in 2× potato. Genome. 34(6): 975-982). The at least one allele can be naturally occurring allele such as, for example, a ps allele. (Mok, D. W. S. & Peloquin, S. J. (1975) Breeding value of 2n pollen (diplandroids) in tetraploid x diploid crosses in potatoes. Theor. Appl. Genet. 46: 307-314; Watanabe K. (2015) Potato genetics, genomics, and applications. Breed Sci. 65(1):53-68). The mutation may be naturally occurring or introduced through gene editing as described in Examples 1 and 2.

The diploid hybrid parent MiMe plant and the diploid homozygous plant are then crossed. Crossing the unreduced (2n, diploid) gametes of the homozygous diploid potato plant with the clonal (2n, diploid) gametes of the parent MiMe plant results in a uniform population of true-breeding, tetraploid seed having the three haplotypes of the parent plants, as described below.

Approximately 20 hand pollinations are performed per cross resulting in roughly 200 seeds each. Seeds of ten crosses are germinated in vitro after surface disinfesting with 10% bleach and rinsing seed with sterile deionized water. The average germination rate per cross is between about 50% and about 95% after a period of dormancy. These crosses are evaluated further via chloroplast counts within guard cells using a light microscope to assess Lugol's iodine-stained epidermal leaf peels (diploid=6-8 chloroplasts per guard cell, tetraploid=12-14 chloroplasts per guard cell). One cross of parent plants, with a germination rate of between about 50% and about 95% and chloroplast counts suggestive of tetraploidy, is selected for further evaluation with flow cytometry.

Using the flow cytometry methods described in Examples 1 and 2, both parent plants are shown to be diploid having DNA content of approximately 1.6-2 pg per 2C nucleus relative to internal controls. Greater than 70% of the 100 progeny of the cross are shown to be tetraploid with DNA content of approximately 3.2-4 pg per 2C nucleus and show similar variability between family members as observed in technical replicates from the same plant.

Genotypic matrices and haplotyping results show specific loci with three alleles in more than 70% of tetraploid members and also show extended haplotype blocks with three haplotypes. After filtering for high quality markers the genotyping matrices for the 100 progeny of the tetraploid are greater than 70% similar.

### Example 11: Tomato

### TM: Methods

### TM1: Plant Materials and Protoplast Isolation

Seeds of diploid Solanum lycopersicum F1 hybrids with pedigree IDs TOM002ASPO, TOM002BSPO, TOM002APAIR and TOM002BPAIR are surface sterilized by incubating in 20% bleach for 20 minutes. Seeds are rinsed five times with sterile distilled water and germinated on MS medium with 3% sucrose. After seed germination (3-5 days), shoot tips are excised and plated in Plant Con containers with 200 mL of TM-1 medium as described by Shahin (1985. Totipotency of tomato protoplasts. Theoretical and Applied Genetics, 69 (3), 235-240) and grown under 16-hour day length under cool fluorescent lights at 25° C. Other diploid Solanum lycopersicum varieties are also selected and treated in the same manner.

After 3 weeks or when expanded leaves are available, plant containers are placed in darkness at 25° C. for 48 hours. After the dark treatment, leaves are cut into small 1-2 mm strips and 1 g of tissue is placed into 30 mL of sterile enzyme solution as described by Shahin (1985). After 4-6 hours of shaking at 40 RPM at 28° C., protoplasts are isolated as described by Shahin (1985). After the isolation steps, cells are quantified using a Bürker hemocytometer and stored at 4° C. in the dark until transfection. A sample is reserved to test cell viability using FDA staining as described by Larkin (1976. “Purification and viability determinations of plant protoplasts.” Planta 128, no. 3: 213-216).

### TM2: Transfection

The protoplasts are transfected with RNPs designed to target MiMe loci identified as described in Example 1's section GM1 and prepared as described in Example 1's section GM2. A MiMe genotype may be generated by targeting (i) REC8; (ii) at least one of OSD1, CYCA1, TDM1; and (iii) at least one of PAIR1 and Spo11-1.

For protoplast transfection, twenty microliters of freshly prepared RNP as described in Example 1's section GM2 are added to the bottom of a 15 mL round bottom tube, after which 100 μL of protoplasts (at a cell density of 1×10⁶ protoplasts/mL) is added and the tube is gently mixed by tapping the tube. Next, 120 μL of 25% PEG solution, as described in Liu et al. (2022. Establishment of a DNA-free genome editing and protoplast regeneration method in cultivated tomato (Solanum lycopersicum). Plant Cell Rep. 41(9): 1843-1852), is added and cells are incubated for 10 minutes at room temperature. Transfections are stopped by adding 1 mL of W5 solution and cells are collected by centrifugation. This washing step is repeated once after which cells are incubated in TM-2 medium from Shahin (1985).

### TM3: Encapsulation and Regeneration of Plants

An equal volume of transfected cells is combined with a 3.2% sodium alginate solution and the solutions are gently mixed together. The resulting solution is pulled into a syringe and dispensed dropwise through a 23-gauge needle into 0.05 M CaCl₂ solution containing 0.4 M D-Mannitol and 5 mM HEPES. Beads are incubated for 30 minutes at room temperature and are then washed with TM-2 medium. Plant regeneration is roughly based on Shahin (1985. Totipotency of tomato protoplasts. Theoretical and Applied Genetics, 69 (3), 235-240). Alginate beads containing protoplasts are cultured in 24 well plates with 2 mL TM-2 medium per well. Plates are sealed with parafilm and incubated at 25C in low light conditions (20 μmol m-2 s-1) at 16 h daylength. After about one week of culture, alginate beads with multicellular colonies are transferred to TM-3 medium (supplemented with 0.2 mg/L 2,4-D and 0.5 mg/L BAP) (TM-3 medium is described in Shahin 1985). After two weeks in TM-3, protoplast derived minicalli are transferred to solid TM-4 medium (also described in Shahin 1985). After 4 weeks on TM-4 medium, shoots start emerging and leaf tissue is collected for genotyping using the methods described in Example 1's section GM3. Regenerated shoots are removed from the callus and placed on TM-5 medium for further shoot development and rooting (TM-5 medium is described in Shahin 1985).

### TM4: Plant Growth and Crossing Conditions

Regenerated plants with confirmed edits in each target gene including REC8, OSD1 or CYCA1 or TDM1, and PAIR1 or SPO11-1 are transplanted from tissue culture to pots containing a peat-based substrate and covered with humidity domes. Plants are kept in a growth chamber at 16-hour daylength and 25° C. for one week until they are transferred to a greenhouse. Knockouts of target genes corresponding to the transfected RNPs are designated TOM002AspoE (rec8/rec8, osd1/osd1 and/or TDM1*/TDM1* and/or cyca1/cyca1, spo11/spo11), TOM002BspoE (rec8/rec8, osd1/osd1 and/or TDM1*/TDM1* and/or cyca1/cyca1, spo11/spo11), TOM002ApairE (rec8/rec8, osd1/osd1 and/or TDM1*/TDM1*and/or cyca1/cyca1, spo11/spo11) and TOM002BpairE (rec8/rec8, osd1/osd1 and/or TDM1*/TDM1* and/or cyca1/cyca1, pair1/pair1), where TDM1* indicates a dominant negative mutation. In the first set of experiments, when plants produce flowers, pollen is collected from the male and manual cross pollination is performed. The cross resulting from TOM002Aspo-E and TOM002Bspo-E is designated TOM002Aspo-002Bspo-E. The cross resulting from TOM002Apair-E and TOM002Bpair-E is designated TOM002Apair-002Bpair-E.

In the second set of experiments, when plants produce flowers, pollen is collected from the male and manual cross pollination is performed. The cross resulting from TOM002Aspo-E and TOM002Bpair-E is designated TOM002Aspo-002Bpair-E. The cross resulting from TOM002Apair-E and TOM002Bspo-E is designated TOM002Apair-002Bspo-E. Seeds are extracted with standard methods and are cleaned and dried for planting.

### TM5: High Density Genotyping of Uniform Polyploid Hybrids

Following seed extraction, 200 cleaned seeds for each family are surface disinfested for 20 minutes in a sonic bath using a ten percent bleach solution containing 20 μL of Tween 20 (Sigma) in a final volume of 50 mL. Disinfested seeds are rinsed twice with sterile deionized water and are germinated in half strength MS minerals with 1% sucrose and placed at 16-hour day length under cool white fluorescent lights. Plants are transferred every three weeks to fresh medium and leaves are removed for DNA extraction during this process.

High quality genomic DNA is extracted from leaves of 100 randomly selected plants from each cross using Qiagen Dneasy Plant Mini kits (69104). Purity is determined using a spectrophotometer and DNA is quantified on a fluorometer as described in earlier Examples. Samples are adjusted to a final concentration of 20 ng/μL and are genotyped following a standard genotyping pipeline for autopolyploids, and then the results are filtered for quality. Genotypic markers specific to the edited parent and indicative of unique haplotypes for TOM002A-E and TOM002B-E are identified and used to determine that the full complement of chromosomes was transmitted to each of the progeny.

### TR: Results

### TR1: Selection of Regenerated Plants

Acclimatized and edited plants derived from TOM002ASPO, TOM002BSPO, TOM002APAIR and TOM002BPAIR are identified using methods described in section GM3 and approximately one percent of plants have mutations predicted to result in nonfunctional proteins or other gene products of all of the genes targeted and therefore having a MiMe genotype. Out of between 2,000 and 10,000 regenerated plants approximately 20 to 100 unique plants are selected for evaluation in a partial diallel crossing scheme.

### TR2: Crossing, Seed Extraction, and Preliminary Ploidy Estimation

Approximately 20 hand pollinations are performed per cross resulting in roughly 200 seeds each. Seeds of ten crosses are germinated in vitro after surface disinfesting with 10% bleach and rinsing seed with sterile distilled water. The average germination rate per cross is between about 50% and about 95%.

### TR3: Flow Cytometry

Edited parents (TOM002Aspo-E, TOM002Apair-E, TOM002Bspo-E, TOM002Bpair-E) are shown to be diploid having DNA contents of approximately 1.6-2 pg per 2C nucleus relative to internal controls. Greater than 70% of the 100 progeny from populations TOM002Aspo-002Bspo-E and TOM002Apair-002Bspo-E are shown to be tetraploid with DNA content of approximately 3.2-4 pg per 2C nucleus and show similar variability between family members as observed in technical replicates from the same plant.

### TR4: Genotypic Evaluation of Uniform Polyploid Hybrids

Genotypic matrices and haplotyping results show specific loci with three or more alleles in more than 70% of tetraploid members in TOM002Aspo-002Bspo-E and TOM002Apair-002Bspo-E and also show extended haplotype blocks with three or more haplotypes. After filtering for high quality markers the genotyping matrices for the 100 progeny of the tetraploid families are greater than 70% similar.

### TR5: Pollen Viability

Pollen from the gene edited diploid parents (TOM002Aspo-E, TOM002Apair-E, TOM002Bspo-E, TOM002Bpair-E) is collected at three time points on two separate days and the pollen viability is at least similar to that of unedited tetraploid parents.

Pollen from individuals from tetraploid populations designated TOM002Aspo-002Bspo-E and TOM002Apair-002Bpair-E is collected at three time points on two separate days. Pollen viability ranges from about 30% to about 70% and is similar to unedited tetraploid plants.

Pollen from individuals from tetraploid populations designated TOM002Aspo-002Bpair-E and TOM002Apair-002Bspo-E is collected at three time points on two separate days. Pollen viability is less than 1%. Low viability is attributed to partial genetic complementation which—eliminates clonal gamete production.

### TR6: Evaluation of Parthenocarpic Fruit

Fruit is examined in hybrid plant populations TOM002Aspo-002Bspo-E and TOM002Apair-002Bpair-E, TOM002Aspo-002Bpair-E and TOM002Apair-002Bspo-E. TOM002Aspo-002Bspo-E and TOM002Apair-002Bpair-E derived fruits have normal seed formation. Fruits from individuals from populations TOM002Aspo-002Bpair-E and TOM002Apair-002Bspo-E are seedless.

### Example 12: Watermelon

### WM: Methods

WM1: Plant Materials for crRNA Screening and Protoplast Isolation

Seeds of diploid accessions of Citrullus lanatus subsp. Vulgaris with pedigree ID CIT002PAIR and pedigree ID CIT002SPO, triploid accessions of Citrullus lanatus with pedigree ID CIT003, and tetraploid accessions of Citrullus lanatus with pedigree ID CIT004, are germinated as described by Yu et al. (2011. “Generation of transgenic watermelon resistant to Zucchini yellow mosaic virus and Papaya ringspot virus type W”. Plant Cell Rep 30:359-371). Briefly, seeds are surface-sterilized and sown on MS solid medium supplemented with 3% sucrose and Schenk & Hildebrandt (SH) vitamins for 3 days at 28° C. in the dark. Other diploid, triploid, or tetraploid Citrullus lanatus or other Citrullus spp. are also selected and treated in the same manner.

Protoplasts are isolated as described by Tian et al. (2017. “Efficient CRISPR/Cas9-based gene knockout in watermelon” Plant Cell Rep 36:399-406). To initiate protoplast isolation, 3- to 5-day-old cotyledons are harvested in sterile manner and sliced into 1 mm strips with a sharp razor blade. The tissue is digested with 10 mL of enzyme solution (1.25% cellulose R10, 0.3% macerozyme R10, 0.4 M mannitol, 20 mM MES, 20 mM KCl, 10 mM CaCl₂, pH 5.7) shaking at 40 RPM for 3-4 h at 25° C. in darkness, and then diluted with 10 ml of W5 solution (2 mM MES, 154 mM NaCl, 125 mM CaCl₂ and 5 mM KCl, pH 5.7). The protoplasts are collected at 100 g for 5 min. The collected protoplasts are filtered using a 21% sucrose solution, after which they are washed again in W5.

### WM2: Transfection

After removal of W5 solution, the protoplasts are diluted in MMG solution (0.4 M Mannitol, 4 mM MES and 15 mM MgCl₂) to a density of 2e⁵ cells/ml. Twenty microliters of freshly prepared RNP as described in section GM2 are added to the bottom of a 15 mL round bottom tube and 100 μL of protoplasts suspended in MMG solution are mixed with the RNP solution. 220 μL of PEG solution (0.2 M Mannitol, 0.1 M CaCl₂ and 40% PEG4000) is added and tubes are gently mixed. The cells are incubated for 7 minutes at 25° C. in darkness. Transfection is stopped by adding 800 μL of W5 solution and the transfected protoplasts are centrifuged at 100 g for 2 minutes. The protoplasts are resuspended in 1 mL of culture medium.

In a first set of experiments, the diploid accession of Citrullus lanatus with pedigree ID CIT003, and the tetraploid accession of Citrullus lanatus with pedigree ID CIT004 are transfected with RNPs targeting REC8, OSD1 or CYCA1, and PAIR1 or SPO11-1.

In a second set of experiments, the diploid accessions of Citrullus lanatus with pedigree ID CIT002PAIR and pedigree ID CIT002SPO are transfected with RNPs targeting different gene sets. CIT002PAIR is transfected with RNPs targeting REC8, OSD1 and/or CYCA1, and PAIR1. CIT002SPO is transfected with RNPs targeting REC8, OSD1 and/or CYCA1, and SPO11-1.

WM3: Encapsulation and regeneration of plants

Transfected protoplasts in culture medium A consisting of a modified B5 medium (B5 salts, KM8p vitamins, 35 mM sucrose, 0.25 M mannitol, 0.17 M glucose, 100 mg/l myo-inositol, 1 g/l glycine, 5.4 μM NAA, 0.9 μM 2,4-D, 2.2 μM BA and 10 ml/l polybuffer) are combined with an equal volume of 3.2% sodium alginate solution. The resulting solution is pulled into a syringe and dispensed dropwise through a 23-gauge needle into 0.05M CaCl₂ solution containing 0.55 M D-Mannitol and 5 mM HEPES. Beads are incubated for 30 minutes at room temperature and are then washed with culture medium.

Plant regeneration is roughly based on Jarl et al. (1995. “Protoplast regeneration and fusion in Cucumis: melon x cucumber” Plant Cell, Tissue and Organ Culture 43: 259-265) and is briefly summarized as follows. After encapsulation alginate beads are cultured in petri dishes containing 20 mL of culture medium A. The petri dishes are incubated in the dark at 28 C. Microcalli can be observed by eye after 3-4 weeks and at this point petri dishes are placed under dim light (22 umol/m/s) at 28° C. for one month. When the minicalli reach 1-2 mm in diameter, they are transferred to shoot initiation medium consisting of MS with 2.3 uM zeatin. After shoot primordia are visible, they are dissected and placed on MS with 0.57 uM IAA and 0.89 uM of BA for further shoot development.

Plants are genotyped using the same sequencing methods described in section GM3. Plants containing complete MiMe genotypes are further subcultured until the shoots have grown sufficiently to be transferred to rooting medium (MS with 0.1 μM NAA, 3% glucose and 0.5% Gelrite™ (Kelco Division of Merck & Co, San Diego, California, USA)).

### WM4: Plant Growth and Crossing Conditions

Regenerated plants with confirmed edits in each target gene including REC8, OSD1 and/or CYCA1, and PAIR1 or SPO11 are pre-hardened off by exchanging unvented lids for vented lids. After two weeks of pre-hardening, rooted plants are removed from tissue culture and transplanted into PROMIX substrate. Plants are grown under greenhouse conditions at about 27° C. and supplemental light is gradually increased over 1 week.

A homozygous diploid or doubled haploid Citrullus plant with pedigree CIT001 is acclimatized and grown under the same conditions.

Acclimatized and edited plants from CIT002SPO, CIT002PAIR, CIT003, and CIT004 are identified using methods described in Example 1's section GM3. Knockouts of target genes corresponding to the transfected RNPs are designated CIT002spoE (rec8/rec8, osd1/osd1, and/or cyca1/cyca1, spo11/spo11), CIT002pairE (rec8/rec8, osd1/osd1 and/or cyca1/cyca1, pair1/pair1), CIT003E (rec8/rec8/rec8, osd1/osd1/osd1 and/or cyca1/cyca1/cyca1, spo11/spo11/spo11 or pair1/pair1/pair1), and CIT004 (rec8/rec8/rec8/rec8, osd1/osd1/osd1/osd1 and/or cyca1/cyca1/cyca1/cyca1, spo11/spo11/spo11/spo11 and/or pair1/pair1/pair1/pair1). The homozygous diploid or doubled haploid Citrullus plant with pedigree CIT001 is screened in the same manner and is determined to be wild type for the genes of interest (REC8/REC8, OSD1/OSD1, CYCA1/CYCA1, SPO11/SPO11, PAIR1/PAIR1). In some plants multiple copies of certain target genes are identified and guides are designed to target these genes in isolation or in combination with one another.

In the first set of experiments, when plants produce flowers (80-100 days after planting), pollen is collected from the male (CIT001, unedited) and cross pollination is performed. The cross resulting from CIT002spoE (female edited) and CIT001 (male) is designated CIT002spoE-001. The cross resulting from CIT003E (female edited) and CIT001 (male) is designated CIT003E-001. The cross resulting from CIT004E (female edited) and CIT001 (male) is designated CIT004E-001. 30-40 days after pollination, fruits are harvested, and seeds are extracted with standard methods. Seeds are cleaned and dried in preparation for planting.

In the second set of experiments, when plants produce flowers (80-100 days after planting) the pollen is collected from CIT002spoE and CIT002pairE. Reciprocal crossing is performed resulting in a population designated CIT002spoE-pairE. 30-40 days after pollination, fruits are harvested and seeds are extracted with standard methods. Seeds are cleaned and dried in preparation for planting.

### WM5: High Density Genotyping

Following seed extraction, 200 cleaned seeds are surface disinfested from each cross for 20 minutes in a sonic bath using a ten percent bleach solution containing 20 microliters of tween 20 (Sigma) in a final volume of 50 mL. Disinfested seeds are rinsed twice with sterile deionized water and germinated on MS medium with 3% sucrose. Plants are transferred every two weeks to fresh medium and leaves are removed for DNA extraction during this process.

High quality genomic DNA is extracted from leaves of 100 randomly selected plants from CIT002spoE-001, CIT002spoE-pairE, CIT003E-001, and CIT004E-001 using Qiagen dNeasy Plant Mini kits (catalog number 69104). Purity is determined using a spectrophotometer and quantified on a fluorometer as described in earlier Examples. Samples are adjusted to a final concentration of 20 ng/μL and are genotyped using Rapid Genomics Flex-Seq™ Services (Gainesville, Florida, USA). Following a standard genotyping pipeline for autopolyploids the resultscare filtered for quality. Genotypic markers specific to the edited parent and indicative of unique haplotypes for CIT001, CIT002spoE, CIT002pairE, CIT003E, and CIT004E are identified and used to determine that the full complement of chromosomes is transmitted to each of the progeny.

### WM6: Hybrid Plant Growth and Pollen Viability

Plants resulting from embryo rescue or direct greenhouse seeding of populations CIT002spoE-001, CIT002spoE-pairE, CIT003E-001, and CIT004E-001 are grown to maturity under standard greenhouse conditions along with clones of parents used to produce each population. After 80-100 days, plants reach maturity and flower for the first time. Pollen collected in the early morning is stained using FDA as described by Heslop-Harrison, J., & Heslop-Harrison, Y. (1970). Evaluation of pollen viability by enzymatically induced fluorescence; intracellular hydrolysis of fluorescein diacetate. Stain technology, 45(3), 115-120. to determine viability.

### WM7: Evaluation of Parthenocarpic Fruit Formation

Following pollen viability tests plants are allowed to undergo normal growth and development. The total number of fruit per plant and seeds per fruit is evaluated for members of CIT002spoE-001, CIT002spoE-pairE, CIT003E-001, and CIT004E-001 as well as the parents of each population. Fruits mature between 30-40 days after flowering and are examined for the presence of seeds.

### WR: Results

### WR1: Selection of Regenerated Plants

Acclimatized and edited plants derived from CIT002SPO, CIT002PAIR, CIT003, and CIT004 are identified using methods described in section GM3 and between one and ten percent of regenerated plants have mutations predicted to result in nonfunctional proteins of REC8, OSD1 or CYCA1 and PAIR1 or in REC8, OSD1 or CYCA1 and SPO11-1. Out of between 2,000 and 10,000 regenerated plants approximately 20 to 100 unique plants are selected for evaluation in a partial diallel crossing scheme. Plants predicted to produce nonfunctional copies of target genes are designated are designated CIT002spoE (rec8/rec8, osd1/osd1, and/or cyca1 spo11/spo11), CIT002pairE (rec8/rec8, osd1/osd1 or cyca1/cyca1, pair1/pair1), CIT003E (rec8/rec8/rec8, osd1/osd1/osd1 or cyca1/cyca1/cyca1, spo11/spo11/spo11 and/or pair1/pair1/pair1), and CIT004E (rec8/rec8/rec8/rec8, osd1/osd1/osd1/osd1 or cyca1/cyca1/cyca1/cyca1, spo11/spo11/spo11/spo11 and/or pair1/pair1/pair1/pair1).

### WR2: Crossing and Embryo Rescue Results

Seeds from CIT002spoE-001, CIT003E-001, and CIT004E-001 are embryo rescued and the success rate ranges per seed lot are from 10-20%, 15-25%, and 15-40% respectively. Some seeds have more developed endosperm and are candidates for direct germination without embryo rescue. CIT004E-001 also produces a larger number of seed per cross relative to CIT003E-001 and CIT002spoE-001. Each cross is evaluated further using flow cytometry.

Seeds from CIT002spoE-pairE have normal endosperm (similar to wild type seeded tetraploid watermelon) and are embryo rescued. Approximately 30-85% of embryos result in viable plants. A portion of the seeds are also directly germinated resulting in 10-50% seed germination under standard greenhouse conditions.

### WR3: Flow Cytometry

Parent CIT001 is shown to be diploid having DNA content of approximately 0.8-1.0 pg per 2C nucleus. CIT002spoE is shown to be diploid having DNA content of approximately 0.8-1.0 pg per 2C nucleus. CIT002pairE is shown to be diploid having DNA content of approximately 0.8-1.0 pg per 2C nucleus. CIT003E is shown to be triploid having DNA content of approximately 1.2-1.5 pg per 2C nucleus. CIT004E is shown to be tetraploid having DNA content of approximately 1.6-2.0 pg per 2C nucleus relative to internal controls.

Greater than 70% of the 100 progeny of CIT002spoE (edited female) and CIT001 in family CIT002spoE-001 are shown to be triploid with DNA content of approximately 1.2-1.5 pg per 2C nucleus and individual measurements show similar variability between family members as observed in technical replicates from the same plant.

Greater than 70% of the 100 progeny of CIT003E (edited female) and CIT001 in family CIT003E-001 are shown to be tetraploid with DNA content of approximately 1.6-2.0 pg per 2C nucleus and individual measurements show similar variability between family members as observed in technical replicates from the same plant.

Greater than 70% of the 100 progeny of CIT004E (edited female) and CIT001 in family CIT004E-001 are shown to be pentaploid with DNA content of approximately 2.0-2.5 pg per 2C nucleus and individual measurements show similar variability between family members as observed in technical replicates from the same plant.

Greater than 70% of the 100 progeny of CIT002spoE and CIT002pairE and the reciprocal cross in family CIT002spoE-pairE are shown to be tetraploid with DNA content of approximately 1.6-2.0 pg per 2C per nucleus and individual measurements show similar variability between family members as observed in technical replicates from the same plant.

### WR4: Genotypic Evaluation of Uniform Polyploid Hybrids

Genotypic matrices and haplotyping results show specific loci with three alleles in more than 70% of triploid members in CIT002E-001 and also show extended haplotype blocks with three haplotypes. After filtering for high quality markers the genotyping matrices for the 100 progeny of the triploid family CIT002E-001 are greater than 70% similar.

Genotypic matrices and haplotyping results show specific loci with three or more alleles in more than 70% of tetraploid members in CIT003E-001 and also show extended haplotype blocks with three or more haplotypes. After filtering for high quality markers the genotyping matrices for the 100 progeny of the tetraploid family CIT003E-001 are greater than 70% similar.

Genotypic matrices and haplotyping results show specific loci with greater than three alleles in more than 70% of pentaploid members in CIT004E-001 and also show extended haplotype blocks with three or more haplotypes. After filtering for high quality markers the genotyping matrices for the 100 progeny of the pentaploid family CIT004E-001 are greater than 70% similar.

Genotypic matrices and haplotyping results show specific loci with greater than three alleles in more than 70% of tetraploid members in CIT002spoE-pairE and also show extended haplotype blocks with three or more haplotypes. After filtering for high quality markers the genotyping matrices for the 100 progeny of the tetraploid family CIT002spoE-pairE are greater than 70% similar.

### WR5: Pollen Viability

Pollen from the diploid plant with pedigree ID CIT001 is collected at three time points on two separate days and the pollen viability ranges from about 30% to about 70%.

Pollen from the diploid plant with pedigree ID CIT002 is collected at three time points on two separate days and the pollen viability ranges from about 30% to about 70%.

Pollen from the triploid plant with pedigree ID CIT003 is collected at three time points on two separate days and the pollen viability is generally less than 1%.

Pollen from the tetraploid plant with pedigree ID CIT004 is collected at three time points on two separate days and the pollen viability ranges from about 30% to about 70%.

Pollen from the gene edited diploid plant with pedigree ID CIT002spoE (rec8/rec8, osd1/osd1 or cyca1/cyca1, spo11/spo11) is collected at three time points on two separate days and the pollen viability ranges from about 30% to about 70%.

Pollen from the gene edited diploid plant with pedigree ID CIT002pairE (rec8/rec8, osd1/osd1 or cyca1/cyca1, pair1/pair1) is collected at three time points on two separate days and the pollen viability ranges from about 30% to about 70%.

Pollen from the gene edited triploid plant with pedigree ID CIT003E (rec8/rec8/rec8, osd1/osd1/osd1 or cyca1/cyca1/cyca1, spo11/spo11/spo11 or pair1/pair1/pair1) is collected at three time points on two separate days and the pollen viability ranges from about 30% to about 70%. High pollen viability in the edited triploid CIT003E relative to the unedited CIT003 is attributed to successful conversion of meiosis to mitosis.

Pollen from the tetraploid plant with pedigree ID CIT004E (rec8/rec8/rec8/rec8, osd1/osd1/osd1/osd1 or cyca1/cyca1/cyca1/cyca1, spo11/spo11/spo11/spo11 or pair1/pair1/pair1/pair) is collected at three time points on two separate days and the pollen viability ranges from about 30% to about 70%.

Pollen from plants of the triploid population designated CIT002spoE-001 is collected at three time points on two separate days. Pollen viability is less than 1%. Low viability relative to the female parent is attributed to genetic complementation which restores the wild type meiosis and eliminates clonal gamete production.

Pollen from the tetraploid population designated CIT003E-001 is collected at three time points on two separate days. Pollen viability ranges from about 30% to about 70%. and is similar to unedited tetraploid plants.

Pollen from the pentaploid population designated CIT004E-001 is collected at three time points on two separate days. Pollen viability is less than 1%. Low viability is attributed to genetic complementation which restores the wild type meiosis and eliminates clonal gamete production.

Pollen from the tetraploid population designated CIT002spoE-pairE is collected at three time points on two separate days. Pollen viability is less than 1%. Low viability is attributed to partial genetic complementation which eliminates clonal gamete production (rec8/rec8/rec8/rec8, osd1/osd1/osd1/osd1 or cyca1/cyca1/cyca1/cyca1, SPO11/SPO11/spo11/spo11, pair1/pair/PAIR1/PAIR1).

### WR6: Evaluation of Parthenocarpic Fruit

Female flowers are pollinated with pollen from the diploid male CIT001. Fruit is examined in hybrid plant populations CIT002spoE-001, CIT003E-001, CIT004E-001, and CIT002spoE-pairE. CIT003E-001 exhibits fruits with normal seed formation. Parthenocarpic fruits form on all other plants and no seeds are observed.

### Example 13: Soybean

### SM: Methods

### SM1: Plant Materials and Protoplat Isolation

Diploid Glycine max F1 hybrids with pedigrees ID SOY002A and SOY002B are germinated in vitro using hormone free Murashige & Skoog Modified BC Potato Medium containing 30 g/L sucrose and grown under 16-hour day with cool white fluorescent lighting. Alternatively, they are grown in the greenhouse with supplemental lighting (16-hour supplemented light per day, 26° C.) Other diploid Glycine max varieties are selected and treated in the same manner.

Immature pods from 60- to 80-day-old plants were collected and surface sterilized with 1% sodium hypochlorite (20% bleach) for 15 min. Young immature cotyledons of 4×2 mm size were dissected from the pods. After removing the seed coat, excised cotyledons were cut transversely into 0.5-1.0 mm thick sections and plasmolyzed for 1 hr in CPW 13% (w/v) mannitol. After two washes with CPW at 13M, approximately 1 g of tissue was incubated in 15 ml of enzyme solution containing 1.5% (w/v) Cellulase ‘Onozuka R10’, 0.2% (w/v) Pectolyase Y-23 and 9% (w/v) mannitol (pH 5.8) in the dark for 4-6 hr with shaking at 50 rpm. Protoplast purification followed the protocol described by Dhir et al. 1991.

Cells are quantified using a Bürker hemocytometer and stored at 4° C. in the dark until transfection. A sample is reserved to test cell viability using FDA staining as described by Larkin (1976. “Purification and viability determinations of plant protoplasts.” Planta 128, no. 3: 213-216).

### SM2: Transfection

The protoplasts are transfected with RNPs designed to target MiMe loci identified as described in Example 1's section GM1 and prepared as described in Example 1's section GM2. A MiMe genotype may be generated by targeting (i) REC8; (ii) at least one of OSD1, CYCA1, TDM1; and (iii) at least one of PAIR1 and Spo11-1.

For protoplast transfection, 20 μL of RNP solution is added to the bottom of a 15 mL tube, after which 100 μL of protoplasts (at a cell density of 1×10⁶ protoplasts/mL) is added and the tube is gently mixed by tapping the tube. Next, 120 μL of PEG solution as described in Wu et al. (2018. A Simple Method for Isolation of Soybean Protoplasts and Application to Transient Gene Expression Analyses. Journal of Visualized Experiments, (131). doi:10.3791/57258).

The solution is gently mixed by inverting and rotating the tube until the solution becomes homogeneous. Tubes are incubated at room temperature for 15 min. Transfections are stopped by slowly adding 400 μL of W5 solution (comprising 154 mM NaCl, 125 mM CaCl₂, and 5 mM KCl) to the 1.5 mL tube at room temperature and gently inverting the tube until the solution becomes homogeneous. The tubes are centrifuged at 100 g for 1-2 min at room temperature and the supernatant is discarded. The washing step is repeated once, and the supernatant is replaced with culture medium A as in Example 12's section WM3.

### SM3: Encapsulation and Regeneration of Plants

An equal volume of transfected cells is combined with a 3.2% sodium alginate solution and the solutions are gently mixed together. The resulting solution is pulled into a syringe and dispensed dropwise through a 23-gauge needle into 0.05 M CaCl₂ solution containing 0.4 M D-Mannitol and 5 mM HEPES. Beads are incubated for 30 minutes at room temperature and are then washed with culture medium.

Plant regeneration is roughly based on Dhir et al. (1992. Regeneration of fertile plants from protoplasts of soybean (Glycine max L. Merr.): genotypic differences in culture response. Plant Cell Reports (1992) 11:285-289) and is briefly summarized as follows. After encapsulation, alginate beads are transferred to 24 well plates with 1 mL of D1 medium (KP8 basal salts with 68.4 g/L glucose, 0.125 g/L sucrose, 0.125 g/L mannitol and 0.125 g/L sorbitol, supplemented with 0.2 mg/L 2,4-D, 1 mg/L NAA and 0.5 mg/L zeatin, 2% Ficoll and 40 mM MES (pH=5.7). Plates were incubated in the dark at 26° C. for 7 days. The osmolarity of the medium was progressively reduced by adding fresh medium or diluting it with KP8 medium. Once calli were 1-2 mm in diameter, they were released from the alginate beads and were subcultured onto M medium with B5 organics, supplemented with 0.5 mg/L each of 2,4-D, BA, kinetin and 500 mg/L CH, 3% sucrose and 0.6% agar. Calli were subcultured 3-4 times at 14-day intervals. When the calli appeared green and compact, they were transferred onto regeneration medium consisting of MS (Murashige and Skoog) salts with Gamborg B5 organics medium with 3% sucrose, 0.5 mg/L each of BA, KN, ZT, 0.1 mg 14 NAA, 500 mg/L CH, 50 mg/L each of asparagine and glutamine and 0.2 mg/L GA3.

Cultures were kept under high intensity light until dark nodular structures appeared on the periphery of calli. These nodular structures were dissected out and gently washed with liquid medium of the same composition to remove dead tissues and phenolic compounds. These selected structures with green bud primordia were transferred onto fresh MSB medium of the same composition for 3-4 subcultures before transferring onto shoot elongation medium as reported earlier (Dhir et al., 1991a). Light intensity was increased gradually throughout the regeneration procedure from 10 pE m2·s˜to 25-30 pE m2·s 4. Elongated shoots of 2-3 cm length were excised and transferred to half strength MS minerals with 1% sucrose and 0.5 mg 1-1 NAA, for root induction.

### SM4: Plant Growth and Crossing Conditions

Regenerated plants with confirmed edits in each target gene, including REC8, OSD1, PAIR1, SPO11-1, CYCA1, are transplanted from tissue culture to pots containing a vermiculite:soil mixture or soil, and these pots were transferred to the greenhouse.

When plants produce flowers, pollen is collected from the male and manual cross pollination is performed. The cross resulting from SOY002A-E and SOY002B-E is designated SOY002A-002B-E. Seeds are extracted with standard methods and are cleaned and dried for planting.

### SM5: High Density Genotyping

Following seed extraction, 200 cleaned seeds are surface disinfested for 20 minutes in a sonic bath using a ten percent bleach solution containing 20 μL of Tween 20 (Sigma) in a final volume of 50 mL. Disinfested seeds are rinsed twice with sterile deionized water and are germinated in half strength MS minerals with 1% sucrose and placed at 16 hour day length under cool white fluorescent bulbs. Plants are transferred every two weeks to fresh medium and leaves are removed for DNA extraction during this process.

High-quality genomic DNA is extracted from leaves of 100 randomly selected plants from FAM051E-013E using Qiagen DNeasy Plant Mini kits (69104). Purity is determined using a spectrophotometer and DNA is quantified on a fluorometer, as described in earlier Examples. Samples are adjusted to a final concentration of 20 ng/μL and are genotyped using Rapid Genomics Flex-Seq™ Services (Gainesville, Florida, USA). Following a standard genotyping pipeline for autopolyploids the results are filtered for quality. Genotypic markers specific to the edited parent and indicative of unique haplotypes for SOY002A-E and SOY002B-E are identified and used to determine that the full complement of chromosomes was transmitted to each of the progeny.

### SR: Results

### SR1: Selection of Regenerated Plants

Regenerated plants are screened using the NGS sequencing methods described above and approximately one percent of plants have mutations predicted to result in nonfunctional proteins or other gene products of all of the genes targeted (and therefore having a MiMe genotype), such as one of the MiMe genotypes described in Table 10. Out of between about 2,000 and about 10,000 regenerated plants approximately 20 to 100 unique plants are selected for evaluation in a partial diallel crossing scheme.

### SR2: Crossing, Seed Extraction, and Preliminary Ploidy Estimation

Approximately 20 hand pollinations are performed per cross, resulting in roughly 200 seeds each. Seeds of ten crosses are germinated in vitro after surface disinfesting with 10% bleach and rinsing seeds with sterile deionized water. The average germination rate per cross is between about 50% and about 95%. These crosses are evaluated further via chloroplast counts within guard cells using a light microscope to assess Lugol's iodine-stained epidermal leaf peels (diploid=4 chloroplasts per guard cell, tetraploid=6-7 chloroplasts per guard cell). One cross of parents designated SOY002A-E and SOY002B-E, with a germination rate of between about 50% and about 95% and chloroplast counts suggestive of tetraploidy, is selected for further evaluation with flow cytometry.

### SR3: Flow Cytometry

Both parents (SOY002A-E and SOY002B-E) are shown to be diploid having DNA content of approximately 2.2 pg per 2C nucleus relative to internal controls. Greater than 70% of the 100 progeny of SOY002A-E and SOY002B-E in SOY002A-002B-E are shown to be tetraploid with DNA content of approximately 4.4 pg per 2C nucleus and show similar variability between family members as observed in technical replicates from the same plant.

### SR4: Genotypic Evaluation

Genotypic matrices and haplotyping results show specific loci with greater than three alleles in more than 70% of tetraploid members in SOY002A-002B-E and also show extended haplotype blocks with three or more haplotypes. After filtering for high quality markers the genotyping matrices for the 100 progeny of the tetraploid family SOY002A-002B-E are greater than 70% similar.

What is claimed is:

1. A method of producing a population of polyploid seed comprising a genetically uniform polyploid seed in an amount of at least 50% of the total number of seeds, the genetically uniform polyploid seed comprising three or more haplotypes of the same or related species of plant, wherein the population was obtained from a single plant or a plurality of F1 hybrids, the method comprising:
(a) generating a first parent MiMe plant, or generating a first parent MiMe plant and a second parent MiMe plant together comprising three or more haplotypes, by introducing one or more genetic modifications and/or expression constructs into one or more candidate lines to produce one or more MiMe alleles at one or more MiMe loci in the germplasm of the first parent MiMe plant, the second parent MiMe plant, or a progenitor thereof, wherein the first parent MiMe plant and the second parent MiMe plant each comprise a complete MiMe genotype comprising only MiMe alleles at each of the one or more MiMe loci, and each of the progenitors comprises a partial MiMe genotype comprising at least one MiMe allele and at least one non-MiMe allele at each of the one or more MiMe loci, wherein if progenitors are generated, the progenitors are further crossed to generate the first parent MiMe plant, the second parent MiMe plant, or both, wherein at least one of the MiMe alleles is introduced by:
(i) introducing genetic modifications in the MiMe locus and selecting for lack of expression at the MiMe locus,
(ii) introducing an expression construct for a silencing RNA targeting the MiMe locus, or
(iii) introducing a natural MiMe allele or selecting the first parent MiMe plant, the second parent MiMe plant, or a progenitor thereof with the natural MiMe allele for use in combination with (i) and/or (ii) for other MiMe alleles;
(b) providing clonal gametes from the first parent MiMe plant;
(c) providing clonal gametes from the second parent MiMe plant, or providing haploid gametes from a homozygous parent non-MiMe plant wherein the first parent MiMe plant and the homozygous parent non-MiMe plant together comprise the three or more haplotypes; and
(d) crossing the clonal gametes from the first and second parent MiMe plants, or crossing the clonal gametes from the first parent MiMe plant with the haploid gametes of the homozygous parent non-MiMe plant, to produce the population of polyploid seed,
wherein the one or more MiMe alleles comprise:
(A)
(1) one or more MiMe alleles at one or more MiMe loci of a component of sister chromatid cohesion during the first division of meiosis comprising a REC8 locus (i.e., a gene encoding a REC8 protein having at least 60% sequence identity to one of SEQ ID NOs: 1-18 and 88),
(2) one or more MiMe alleles at one or more MiMe loci of a component of DNA double strand breakage during meiotic recombination comprising a PAIR1 locus (i.e., a gene encoding a PAIR1 protein having at least 60% sequence identity to one of SEQ ID NOs: 37-55 and 90), a SPO11-1 locus (i.e., a gene encoding a SPO11-1 protein having at least 60% sequence identity to one SEQ ID NOs: 19-36 and 89), or a combination thereof, and
(3) a natural os allele or one or more MiMe alleles at one or more MiMe loci of a component of progression through the second division of meiosis comprising an OSD1 locus (i.e., a gene encoding an OSD1 protein having at least 60% sequence identity to one of SEQ ID NOs: 56-75, 91, and 92), a CYCA1 locus (i.e., a gene encoding a CYCA1 protein having at least 60% sequence identity to one of SEQ ID NOs: 78, 81-83, 93, and 96-119), a TDM1 locus (i.e., a gene encoding a TDM1 protein having at least 60% sequence identity to one of SEQ ID NOs: 79, 84, 94, and 95), or any combination thereof; or
(B)
(2) one or more MiMe alleles at one or more MiMe loci of a component of DNA double strand breakage during meiotic recombination comprising a PAIR1 locus (i.e., a gene encoding a PAIR1 protein having at least 60% sequence identity to one of SEQ ID NOs: 37-55 and 90), a SPO11-1 locus (i.e., a gene encoding a SPO11-1 protein having at least 60% sequence identity to one SEQ ID NOs: 19-36 and 89), or a combination thereof, and
(4) a natural ps allele or one or more MiMe alleles at one or more MiMe loci of a component of progression through the first division of meiosis comprise a PS1 locus (i.e., a gene encoding a PS1 protein having at least 60% sequence identity to one of SEQ ID NOs: 80, 85, and 86), a JASON locus (i.e., a gene encoding a JASON protein having at least 60% sequence identity to one of SEQ ID NOs: 76, 77, and 87), or a combination thereof;
wherein the genetically uniform polyploid seeds comprises;
(i) a complete MiMe genotype comprising only MiMe alleles at each of the one or more MiMe loci,
(ii) a partial MiMe genotype comprising at least one MiMe allele and at least one non-MiMe allele at each of the one or more MiMe loci, or
(iii) a partially complemented MiMe genotype comprising only MiMe alleles at a first MiMe locus of the one or more MiMe loci and at least one MiMe allele and at least one non-MiMe allele at a second MiMe locus of the one or more MiMe loci.

2. The method of claim 1, wherein the genetically uniform polyploid seed is triploid, tetraploid, pentaploid, hexaploid, heptaploid, or octoploid.

3. The method of claim 1, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.

4. The method of claim 1, wherein the population of polyploid seed comprises the genetically uniform polyploid seed in an amount of at least 70%, at least 90%, at least 95%, at least 96%, at least 97%, at least 98%, or at least 99% of the total number of seeds.

5. The method of claim 1, wherein each pair of seeds in the genetically uniform polyploid seed has a pairwise identity of at least 95%, at least 96%, at least 97%, at least 98%, at least 99%, or 100% as measured by the Jaccard similarity coefficient.

6. (canceled)

7. The method of claim 1, wherein the genetically uniform polyploid seed comprises:
(1) a complete MiMe genotype comprising (i) only MiMe alleles at the OSD1 locus, wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(2) a partial MiMe genotype comprising (i) at least one MiMe allele and at least one non-MiMe allele at the OSD1 locus, wherein each of the MiMe alleles at the OSD1 locus comprise one or more genetic modifications resulting in decreased expression of the OSD1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(3) a complete MiMe genotype comprising (i) only MiMe alleles at the OSD1 locus, wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(4) a partial MiMe genotype comprising (i) at least one MiMe allele and at least one non-MiMe allele at the OSD1 locus, wherein each of the MiMe alleles at the OSD1 locus comprise one or more genetic modifications resulting in decreased expression of the OSD1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(5) a complete MiMe genotype comprising (i) only MiMe alleles at the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein P044-4;
(6) a partial MiMe genotype comprising (i) at least one MiMe allele and at least one non-MiMe allele at the CYCA1 locus, wherein each of the MiMe alleles at the CYCA1 locus comprise one or more genetic modifications resulting in decreased expression of the CYCA1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(7) a complete MiMe genotype comprising (i) only MiMe alleles a the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(8) a partial MiMe genotype comprising (i) at least one MiMe allele and at least one non-MiMe allele at the CYCA1 locus, wherein each of the MiMe alleles at the CYCA1 locus comprise one or more genetic modifications resulting in decreased expression of the CYCA1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(9) a complete MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(10) a partial MiMe genotype comprising (i) at least one MiMe allele and at least one non-MiMe allele at the TDM1 locus, wherein each of the MiMe alleles at the TDM1 locus comprise one or more genetic modifications resulting in decreased expression of the TDM1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(11) a complete MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(12) a partial MiMe genotype comprising (i) at least one MiMe allele and at least one non-MiMe allele at the TDM1 locus, wherein each of the MiMe alleles at the TDM1 locus comprise one or more genetic modifications resulting in decreased expression of the TDM1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(13) a complete MiMe genotype comprising (i) the natural os allele, wherein the genetically uniform polyploid seed is homozygous for the natural os allele, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1;
(14) a partial MiMe genotype comprising (i) the natural os allele, wherein the genetically uniform polyploid seed is heterozygous for the natural os allele, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(15) a complete MiMe genotype comprising (i) the natural ps allele, wherein the genetically uniform polyploid seed is homozygous for the natural ps allele, and (ii) only MiMe alleles at the SPO11-1 locus, wherein each of the MiMe alleles at the SPO11-1 locus comprise one or more genetic modifications resulting in decreased expression of the SPO11-1;
(16) a partial MiMe genotype comprising (i) the natural ps allele, wherein the genetically uniform polyploid seed is heterozygous for the natural ps allele, and (ii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(17) a partially complemented MiMe genotype comprising (i) only MiMe alleles at the OSD1 locus wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein,
(18) a partially complemented MiMe genotype comprising (i) only MiMe alleles at the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus mere PAIR1 lee comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(19) a partially complemented MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus mere PAIR1 lee comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(20) a partially complemented MiMe genotype comprising (i) the natural ps allele, wherein the genetically uniform polyploid seed is heterozygous for the natural ps allele, (ii) the natural os allele, wherein the genetically uniform polyploid seed is heterozygous for the natural os allele, (iii) at least one MiMe allele and at least one non-MiMe allele at the REC8 locus, wherein each of the MiMe alleles at the REC8 locus comprise one or more genetic modifications resulting in decreased expression of the REC8 protein, and (iv) only MiMe alleles at the SPO11-1 locus, wherein each of the MiMe alleles at the SPO11-1 locus comprise one or more genetic modifications resulting in decreased expression of the SPO11-1 protein; or
(21) a partially complemented MiMe genotype comprising (i) the natural os allele, wherein the genetically uniform polyploid seed is heterozygous for the natural os allele, (ii) at least one MiMe allele and at least one non-MiMe allele at the CYCA1 locus, wherein each of the MiMe alleles at the CYCA1 locus comprise one or more genetic modifications resulting in decreased expression of the CYCA1 protein, (iii) only MiMe alleles at the REC8 locus, wherein each of the MiMe alleles at the REC8 locus comprise one or more genetic modifications resulting in decreased expression of the REC8 protein, and (iv) only MiMe alleles at the SPO11-1 locus, wherein each of the MiMe alleles at the SPO11-1 locus comprise one or more genetic modifications resulting in decreased expression of the SPO11-1 protein.

8. The method of claim 1, wherein the population of polyploid seed is from a plant selected from the group consisting of potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, ashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussel sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine, mandarin, clementine, coffee, cola, hazelnut, saffron, melon, cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycrass, green bean, bean, ground cherry, pine, pistachio nut, pea, turf grass, poplar, apricot, plum, prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.

9. A population of polyploid seed comprising genetically uniform polyploid seed in an amount of at least 50% of the total number of seeds, the genetically uniform polyploid seed comprising three or more haplotypes of the same or related species of plant, wherein the population was obtained from a single plant or a plurality of F1 hybrids,
wherein the population of polyploid seed is from a cross of a first parent MiMe plant with a second parent MiMe plant, or from a cross of a first parent MiMe plant with a homozygous non-MiMe parent plant,
wherein the first parent MiMe plant, the second parent MiMe plant, or both were generated by introducing one or more genetic modifications and/or expression constructs into one or more candidate lines to produce one or more MiMe alleles at one or more MiMe loci in the germplasm of the first parent MiMe plant, the second parent MiMe plant, or a progenitor thereof, wherein the first parent MiMe plant and the second parent MiMe plant each comprise a complete MiMe genotype comprising only MiMe alleles at each of the one or more MiMe loci, and each of the progenitors comprises a partial MiMe genotype comprising at least one MiMe allele and at least one non-MiMe allele at each of the one or more MiMe loci, wherein if progenitors were generated, the progenitors were further crossed to generate the first parent MiMe plant, the second parent MiMe plant, or both,
wherein at least one of the MiMe alleles was introduced by:
(i) introducing genetic modifications in the MiMe locus and selecting for lack of expression at the MiMe locus,
(ii) introducing an expression construct for a silencing RNA targeting the MiMe locus, or
(iii) introducing a natural MiMe allele or selecting the first parent MiMe plant, the second parent MiMe plant, or a progenitor thereof with the natural MiMe allele for use in combination with ( ) and/or (ii) for other MiMe alleles
wherein the one or more MiMe alleles comprise:
(A)
(1) one or more MiMe alleles at one or more MiMe loci of a component of sister chromatid cohesion during the first division of meiosis comprising a REC8 locus (i.e., a gene encoding a REC8 protein having at least 60% sequence identity to one of SEQ ID NOs: 1-18 and 88),
(2) one or more MiMe alleles at one or more MiMe loci of a component of DNA double strand breakage during meiotic recombination comprising a PAIR1 locus (i.e., a gene encoding a PAIR1 protein having at least 60% sequence identity to one of SEQ ID NOs: 37-55 and 90), a SPO11-1 locus (i.e., a gene encoding a SPO11-1 protein having at least 60% sequence identity to one SEQ ID NOs: 19-36 and 89), or a combination thereof, and
(3) a natural os allele or one or more MiMe alleles at one or more MiMe loci of a component of progression through the second division of meiosis comprising an OSD1 locus (i.e., a gene encoding an OSD1 protein having at least 60% sequence identity to one of SEQ ID NOs: 56-75, 91, and 92), a CYCA1 locus (i.e., a gene encoding a CYCA1 protein having at least 60% sequence identity to one of SEQ ID NOs: 78, 81-83, 93, and 96-119), a TDM1 locus (i.e., a gene encoding a TDM1 protein having at least 60% sequence identity to one of SEQ ID NOs: 79, 84, 94, and 95), or any combination thereof; or
(B)
(2) one or more MiMe alleles at one or more MiMe loci of a component of DNA double strand breakage during meiotic recombination comprising a PAIR1 locus (i.e., a gene encoding a PAIR1 protein having at least 60% sequence identity to one of SEQ ID NOs: 37-55 and 90), a SPO11-1 locus (i.e., a gene encoding a SPO11-1 protein having at least 60% sequence identity to one SEQ ID NOs: 19-36 and 89), or a combination thereof, and
(4) a natural ps allele or one or more MiMe alleles at one or more MiMe loci of a component of progression through the first division of meiosis comprise a PS1 locus (i.e., a gene encoding a PS1 protein having at least 60% sequence identity to one of SEQ ID NOs: 80, 85, and 86), a JASON locus (i.e., a gene encoding a JASON protein having at least 60% sequence identity to one of SEQ ID NOs: 76, 77, and 87), or a combination thereof;
and wherein the genetically uniform polyploid seed comprises
(i) a complete MiMe genotype comprising only MiMe alleles at each of the one or more MiMe loci,
(ii) a partial MiMe genotype comprising at least one MiMe allele and at least one non-MiMe allele at each of the one or more MiMe loci, or
(iii) a partially complemented MiMe genotype comprising only MiMe alleles at a first MiMe locus of the one or more MiMe loci and at least one MiMe allele and at least one non-MiMe allele at a second MiMe locus of the one or more MiMe loci.

10. The population of claim 9, wherein the genetically uniform polyploid seed is triploid, tetraploid, pentaploid, hexaploid, heptaploid, or octoploid.

11. The population of claim 9, wherein the population of polyploid seed has an average pairwise genetic uniformity of at least 80%, at least 85%, at least 90%, at least 95%, at least 96%, at least 97%, at least 98%, at least 99%, or 100% as measured by the Jaccard similarity coefficient.

12. The population of claim 9, wherein the population of polyploid seed comprises the genetically uniform polyploid seed in an amount of at least 70%, at least 90%, at least 95%, at least 96%, at least 97%, at least 98%, or at least 99% of the total number of seeds.

13. The population of claim 9, wherein each pair of seeds in the genetically uniform polyploid seed has a pairwise identity of at least 95%, at least 96%, at least 97%, at least 98%, at least 99%, or 100% as measured by the Jaccard similarity coefficient.

14. (canceled)

15. The population of claim 9, wherein the genetically uniform polyploid seed comprises:
(1) a complete MiMe genotype comprising (i) only MiMe alleles at the OSD1 locus, wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(2) a partial MiMe genotype comprising (i) at least one MiMe allele and at least one non-MiMe allele at the OSD1 locus, wherein each of the MiMe alleles at the OSD1 locus comprise one or more genetic modifications resulting in decreased expression of the OSD1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(3) a complete MiMe genotype comprising (i) only MiMe alleles at the OSD1 locus, wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(4) a partial MiMe genotype comprising (i) at least one MiMe allele and at least one non-MiMe allele at the OSD1 locus, wherein each of the MiMe alleles at the OSD1 locus comprise one or more genetic modifications resulting in decreased expression of the OSD1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(5) a complete MiMe genotype comprising (i) only MiMe alleles at the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(6) a partial MiMe genotype comprising (i) at least one MiMe allele and at least one non-MiMe allele at the CYCA1 locus, wherein each of the MiMe alleles at the CYCA1 locus comprise one or more genetic modifications resulting in decreased expression of the CYCA1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(7) a complete MiMe genotype comprising (i) only MiMe alleles at the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(8) a partial MiMe genotype comprising (i) at least one MiMe allele and at least one non-MiMe allele at the CYCA1 locus, wherein each of the MiMe alleles at the CYCA1 locus comprise one or more genetic modifications resulting in decreased expression of the CYCA1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(9) a complete MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(10) a partial MiMe genotype comprising (i) at least one MiMe allele and at least one non-MiMe allele at the TDM1 locus, wherein each of the MiMe alleles at the TDM1 locus comprise one or more genetic modifications resulting in decreased expression of the TDM1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(11) a complete MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(12) a partial MiMe genotype comprising (i) at least one MiMe allele and at least one non-MiMe allele at the TDM1 locus, wherein each of the MiMe alleles at the TDM1 locus comprise one or more genetic modifications resulting in decreased expression of the TDM1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(13) a complete MiMe genotype comprising (i) the natural os allele, wherein the genetically uniform polyploid seed is homozygous for the natural os allele, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(14) a partial MiMe genotype comprising (i) the natural os allele, wherein the genetically uniform polyploid seed is heterozygous for the natural os allele, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(15) a complete MiMe genotype comprising (i) the natural ps allele, wherein the genetically uniform polyploid seed is homozygous for the natural ps allele, and (ii) only MiMe alleles at the SPO11-1 locus, wherein each of the MiMe alleles at the SPO11-1 locus comprise one or more genetic modifications resulting in decreased expression of the SPO11-1 protein;
(16) a partial MiMe genotype comprising (i) the natural ps allele, wherein the genetically uniform polyploid seed is heterozygous for the natural ps allele, and (ii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(17) a partially complemented MiMe genotype comprising (i) only MiMe alleles at the OSD1 locus, wherein each of the MiMe alleles at the OSD1 locus mere OSD1-loci comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(18) a partially complemented MiMe genotype comprising (i) only MiMe alleles at the CYCA1 locus wherein each of the MiMe alleles at the CYCA1 locus comprise one or more genetic modifications resulting in decreased expression of the CYCA1 protein, (ii) only MiMe alleles at the REC8 locus mere REC-8 lee, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(19) a partially complemented MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(20) a partially complemented MiMe genotype comprising (i) the natural ps allele, wherein the genetically uniform polyploid seed is heterozygous for the natural ps allele, (ii) the natural os allele, wherein the genetically uniform polyploid seed is heterozygous for the natural os allele, (iii) at least one MiMe allele and at least one non-MiMe allele at the REC8 locus, wherein each of the MiMe alleles at the REC8 locus comprise one or more genetic modifications resulting in decreased expression of the REC8 protein, and (iv) only MiMe alleles at the SPO11-1 locus, wherein each of the MiMe alleles at the SPO11-1 locus comprise one or more genetic modifications resulting in decreased expression of the SPO11-1 protein; or
(21) a partially complemented MiMe genotype comprising (i) the natural os allele, wherein the genetically uniform polyploid seed is heterozygous for the natural os allele, (ii) at least one MiMe allele and at least one non-MiMe allele at the CYCA1 locus, wherein each of the MiMe alleles at the CYCA1 locus comprise one or more genetic modifications resulting in decreased expression of the CYCA1 protein, (iii) only MiMe alleles at the REC8 locus, wherein each of the MiMe alleles at the REC8 locus comprise one or more genetic modifications resulting in decreased expression of the REC8 protein, and (iv) only MiMe alleles at the SPO11-1 locus, wherein each of the MiMe alleles at the SPO11-1 locus comprise one or more genetic modifications resulting in decreased expression of the SPO11-1 protein.

16. The population of claim 9, wherein the population of polyploid seed is from a plant selected from the group consisting of potato, maize, banana, blueberry, blackberry, watermelon, muskmelon, tomato, tomatillo, pepper, eggplant, grape, orange, lemon, lime, grapefruit, cucumbers, squash, gourd, pumpkin, apple, pear, kiwi, pomegranate, mango, guava, Papaya, avocado, stone fruit, date, fig, alfalfa, tobacco, cotton, clover, strawberry, currant, cranberry, gooseberry, boysenberry, raspberry, artichoke, beets, potato, sweet potato, achira, ahipa, arracacha, maca, nashua, mauka, oca, ulluco, yacon, yams, radish, horseradish, turnip, parsnip, rutabaga, Yucca, maize, onion, shallot, leek, scallion, garlic, chives, peanut, asparagus, sugarcane, cassava, brussel sprouts, cabbage, collards, kale, lettuce, chard, spinach, bok choy, okra, cashew nuts, pineapple, celery, oat, birch, rapeseed, mustard, tea, hemp, safflower seed, cedar, Quinoa, chickpea, citron, satsuma, tangerine, mandarin, clementine, coffee, cola, hazelnut, saffron, melon, cantaloupe, carrot, oil palms, teff, rubber rabbit brush, Eucalyptus, fir, soybean, sunflower, hemlock tree, rubber tree, kenaf, barley, hop, walnut, larch, lentil, flax, ryegrass, maple, Miscanthus, basil, olive, rice, millet, pennycrass, green bean, bean, ground cherry, pine, pistachio nut, pea, turf grass, poplar, apricot, plum, prune, almond, nectarine, peach, cherry, rose, Rubus, rye, sesame, sorghum, spruce, switchgrass, Russian dandelion, cacao, durum wheat, spelt, wheat, broad bean, cowpea, ginger, kohlrabi, broccoli and cauliflower.

17. A method of breeding a polyploid hybrid plant line, the method comprising:
(a) obtaining a set of lines of a plant;
(b) breeding the lines using traditional plant breeding methods to produce a set of candidate lines of the plant;
(c) selecting two or more candidate lines together comprising three or more haplotypes;
(d) generating a first parent MiMe plant, or generating a first parent MiMe plant and a second parent MiMe plant, from the two or more candidate lines that together comprise the three or more haplotypes, by introducing one or more genetic modifications and/or expression constructs into the two or more candidate lines to produce one or more MiMe alleles at one or more MiMe loci in the germplasm of the first parent MiMe plant, the second parent MiMe plant, or a progenitor thereof, wherein the first parent MiMe plant and the second parent MiMe plant each comprise a complete MiMe genotype comprising only MiMe alleles at each of the one or more MiMe loci, and each of the progenitors comprises a partial MiMe genotype comprising at least one MiMe allele and at least one non-MiMe allele at each of the one or more MiMe loci, wherein if progenitors are generated, the progenitors are further crossed to generate the first parent MiMe plant, the second parent MiMe plant, or both, wherein at least one of the MiMe alleles is introduced by:
(i) introducing genetic modifications in the MiMe locus and selecting for lack of expression at the MiMe locus,
(ii) introducing an expression construct for a silencing RNA targeting the MiMe locus, or
(iii) introducing a natural MiMe allele or selecting the first parent MiMe plant, the second parent MiMe plant, or a progenitor thereof with the natural MiMe allele for use in combination with (i) and/or (ii) for other MiMe alleles;
(e) providing clonal gametes from the first parent MiMe plant;
(f) providing clonal gametes from the second parent MiMe plant, or providing haploid gametes from a homozygous parent non-MiMe plant of one of the two or more candidate lines wherein the first parent MiMe plant and the homozygous parent non-MiMe plant together comprises three or more haplotypes;
(f) crossing the clonal gametes from the first and second parent MiMe plants, or crossing the clonal gametes from the first parent MiMe plant with the haploid gametes from the homozygous parent non-MiMe plant, to produce a hybrid polyploid seed comprising the three or more haplotypes;
(g) growing the hybrid polyploid seed to produce a hybrid polyploid plant comprising three or more haplotypes; and
(h) evaluating one or more characteristics of the hybrid polyploid plant, wherein the first parent MiMe plant and the homozygous parent non-MiMe plant together comprise three or more haplotypes, and wherein the growing of step (g) results in the hybrid polyploid plant comprising three or more haplotypes;
wherein the one or more MiMe alleles comprise:
(A)
(1) one or more MiMe alleles at one or more MiMe loci of a component of sister chromatid cohesion during the first division of meiosis comprising a REC8 locus (i.e., a gene encoding a REC8 protein having at least 60% sequence identity to one of SEQ ID NOs: 1-18 and 88),
(2) one or more MiMe alleles at one or more MiMe loci of a component of DNA double strand breakage during meiotic recombination comprising a PAIR1 locus (i.e., a gene encoding a PAIR1 protein having at least 60% sequence identity to one of SEQ ID NOs: 37-55 and 90), a SPO11-1 locus (i.e., a gene encoding a SPO11-1 protein having at least 60% sequence identity to one SEQ ID NOs: 19-36 and 89), or a combination thereof, and
(3) a natural os allele or one or more MiMe alleles at one or more MiMe loci of a component of progression through the second division of meiosis comprising an OSD1 locus (i.e., a gene encoding an OSD1 protein having at least 60% sequence identity to one of SEQ ID NOs: 56-75, 91, and 92), a CYCA1 locus (i.e., a gene encoding a CYCA1 protein having at least 60% sequence identity to one of SEQ ID NOs: 78, 81-83, 93, and 96-119), a TDM1 locus (i.e., a gene encoding a TDM1 protein having at least 60% sequence identity to one of SEQ ID NOs: 79, 84, 94, and 95), or any combination thereof; or
(B)
(2) one or more MiMe alleles at one or more MiMe loci of a component of DNA double strand breakage during meiotic recombination comprising a PAIR1 locus (i.e., a gene encoding a PAIR1 protein having at least 60% sequence identity to one of SEQ ID NOs: 37-55 and 90), a SPO11-1 locus (i.e., a gene encoding a SPO11-1 protein having at least 60% sequence identity to one SEQ ID NOs: 19-36 and 89), or a combination thereof, and
(4) a natural ps allele or one or more MiMe alleles at one or more MiMe loci of a component of progression through the first division of meiosis comprise a PS1 locus (i.e., a gene encoding a PS1 protein having at least 60% sequence identity to one of SEQ ID NOs: 80, 85, and 86), a JASON locus (i.e., a gene encoding a JASON protein having at least 60% sequence identity to one of SEQ ID NOs: 76, 77, and 87), or a combination thereof;
wherein the hybrid polyploid seed comprises
(i) a complete MiMe genotype comprising only MiMe alleles at each of the one or more MiMe loci,
(ii) a partial MiMe genotype comprising at least one MiMe allele and at least one non-MiMe allele at each of the one or more MiMe loci, or
(iii) a partially complemented MiMe genotype comprising only MiMe alleles at a first MiMe locus of the one or more MiMe loci and at least one MiMe allele and at least one non-MiMe allele at a second MiMe locus of the one or more MiMe loci.

18. The method of claim 17, wherein the first parent MiMe plant, the second parent MiMe plant, the parent non-MiMe plant, or any combination thereof are diploid, triploid, or tetraploid and the hybrid polyploid plant is tetraploid, pentaploid, hexaploid, heptaploid, or octoploid.

19. The method of claim 17, wherein generating the first parent MiMe plant, the second parent MiMe plant, or both comprises:
(1) introducing a complete MiMe genotype directly into two candidate lines to produce the first parent MiMe plant, the second parent MiMe plant, or both;
(2) introducing a partial MiMe genotype into two candidate lines to produce two grandparent non-MiMe plants each having a partial MiMe genotype and crossing the grandparent non-MiMe plants each having a partial MiMe genotype to produce the first parent MiMe plant; or
(3) introducing a partial MiMe genotype into four candidate lines to produce four grandparent non-MiMe plants each having a partial MiMe genotype, and crossing pairs of said grandparent non-MiMe plants each having a partial MiMe genotype to produce the first and second parent MiMe plants.

20. The method of claim 17, wherein the hybrid polyploid plant comprises:
(1) a complete MiMe genotype comprising (i) only MiMe alleles at the OSD1 locus, wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(2) a partial MiMe genotype comprising (i) at least one MiMe allele and at least one non-MiMe allele at the OSD1 locus, wherein each of the MiMe alleles at the OSD1 locus comprise one or more genetic modifications resulting in decreased expression of the OSD1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(3) a complete MiMe genotype comprising (i) only MiMe alleles at the OSD1 locus, wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(4) a partial MiMe genotype comprising (i) at least one MiMe allele and at least one non-MiMe allele at the OSD1 locus, wherein each of the MiMe alleles at the OSD1 locus comprise one or more genetic modifications resulting in decreased expression of the OSD1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(5) a complete MiMe genotype comprising (i) only MiMe alleles at the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein-4;
(6) a partial MiMe genotype comprising (i) at least one MiMe allele and at least one non-MiMe allele at the CYCA1 locus, wherein each of the MiMe alleles at the CYCA1 locus comprise one or more genetic modifications resulting in decreased expression of the CYCA1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(7) a complete MiMe genotype comprising (i) only MiMe alleles at the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(8) a partial MiMe genotype comprising (i) at least one MiMe allele and at least one non-MiMe allele at the CYCA1 locus, wherein each of the MiMe alleles at the CYCA1 locus comprise one or more genetic modifications resulting in decreased expression of the CYCA1 protein, (ii) at least one MiMe allele and at least one non-MiMe allele at the REC8 locus wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(9) a complete MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(10) a partial MiMe genotype comprising (i) at least one MiMe allele and at least one non-MiMe allele at the TDM1 locus, wherein each of the MiMe alleles at the TDM1 locus comprise one or more genetic modifications resulting in decreased expression of the TDM1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(11) a complete MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, and (iii) only MiMe alleles at the PAIR1 locus, wherein each of the MiMe alleles at the PAIR1 locus comprise one or more genetic modifications resulting in decreased expression of the PAIR1 protein;
(12) a partial MiMe genotype comprising (i) at least one MiMe allele and at least one non-MiMe allele at the TDM1 locus, wherein each of the MiMe alleles at the TDM1 locus comprise one or more genetic modifications resulting in decreased expression of the TDM1 protein, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein;
(13) a complete MiMe genotype comprising (i) the natural os allele, wherein the hybrid polyploid plant is homozygous for the natural os allele, (ii) only MiMe alleles at the REC8 locus, wherein each of the MiMe alleles at the REC8 locus comprise one or more genetic modifications resulting in decreased expression of the REC8 protein, and (iii) only MiMe alleles at the SPO11-1 locus, wherein each of the MiMe alleles at the SPO11-1 locus comprise one or more genetic modifications resulting in decreased expression of the SPO11-1 protein;
(14) a partial MiMe genotype comprising (i) the natural os allele, wherein the hybrid polyploid plant is heterozygous for the natural os allele, (ii) at least one MiMe allele and at least one non-MiMe allele at the REC8 locus, wherein each of the MiMe alleles at the REC8 locus comprise one or more genetic modifications resulting in decreased expression of the REC8 protein, and (iii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(15) a complete MiMe genotype comprising (i) the natural ps allele, wherein the hybrid polyploid plant is homozygous for the natural ps allele, and (ii) only MiMe alleles at the SPO11-1 locus, wherein each of the MiMe alleles at the SPO11-1 locus comprise one or more genetic modifications resulting in decreased expression of the SPO11-1 protein;
(16) a partial MiMe genotype comprising (i) the natural ps allele, wherein the hybrid polyploid plant is heterozygous for the natural ps allele, and (ii) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(17) a partially complemented MiMe genotype comprising (i) only MiMe alleles at the OSD1 locus, wherein each of the MiMe alleles at the OSD1 locus comprise one or more genetic modifications resulting in decreased expression of the OSD1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(18) a partially complemented MiMe genotype comprising (i) only MiMe alleles at the CYCA1 locus, wherein each of the MiMe alleles at the CYCA1 locus comprise one or more genetic modifications resulting in decreased expression of the CYCA1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus mere PAIR1 lee comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(19) a partially complemented MiMe genotype comprising (i) only MiMe alleles at the TDM1 locus, wherein each of the MiMe alleles at the TDM1 locus comprise one or more genetic modifications resulting in decreased expression of the TDM1 protein, (ii) only MiMe alleles at the REC8 locus, wherein each of the MiMe alleles at the REC8 locus comprise one or more genetic modifications resulting in decreased expression of the REC8 protein, (iii) at least one MiMe allele and at least one non-MiMe allele at the PAIR1 locus, wherein each of the MiMe alleles at the PAIR1 locus comprise one or more genetic modifications resulting in decreased expression of the PAIR1 protein, and (iv) at least one MiMe allele and at least one non-MiMe allele at the SPO11-1 locus, wherein each of the MiMe alleles at the SPO11-1 locus comprise one or more genetic modifications resulting in decreased expression of the SPO11-1 protein;
(20) a partially complemented MiMe genotype comprising (i) the natural ps allele, wherein the hybrid polyploid plant is heterozygous for the natural ps allele, (ii) the natural os allele, wherein the hybrid polyploid plant is heterozygous for the natural os allele, (iii) at least one MiMe allele and at least one non-MiMe allele at the REC8 locus, wherein each of the MiMe alleles at the REC8 locus comprise one or more genetic modifications resulting in decreased expression of the REC8 protein, and (iv) only MiMe alleles at the SPO11-1 locus, wherein each of the MiMe alleles at the SPO11-1 locus comprise one or more genetic modifications resulting in decreased expression of the SPO11-1 protein; or
(21) a partially complemented MiMe genotype comprising (i) the natural os allele, wherein the hybrid polyploid plant is heterozygous for the natural os allele, (ii) at least one MiMe allele and at least one non-MiMe allele at the CYCA1 locus, wherein each of the MiMe alleles at the CYCA1 locus comprise one or more genetic modifications resulting in decreased expression of the CYCA1 protein, (iii) only MiMe alleles at the REC8 locus, wherein each of the MiMe alleles at the REC8 locus comprise one or more genetic modifications resulting in decreased expression of the REC8 protein, and (iv) only MiMe alleles at the SPO11-1 locus, wherein each of the MiMe alleles at the SPO11-1 locus comprise one or more genetic modifications resulting in decreased expression of the SPO11-1 protein.

21. The method of claim 17, wherein the MiMe alleles are introduced by gene editing, transgenesis, or a combination thereof.

22. The method of claim 17, wherein introducing the MiMe alleles comprises introducing into each candidate line an insertion, a deletion, one or more nucleotide changes, or an inversion that eliminates expression of the MiMe locus.

23. The method of claim 22, wherein the expression of the MiMe locus is eliminated by a premature stop codon present in the 70%, the first 60%, the first 50%, the first 40%, the first 30%, the first 20%, or the first 10% of the nucleotides of the coding sequence of the MiMe locus following the start codon in the 3′ direction.

24-27. (canceled)

28. A genetically modified plant, plant part, or plant cell comprising:
(A) three or more haplotypes; and
(B)
(a) (1) one or more MiMe alleles at each of one or more MiMe loci of a component of sister chromatid cohesion during the first division of meiosis comprising REC8, SWITCH1/DYAD, or a combination thereof, (2) one or more MiMe alleles at each of one or more MiMe loci of a component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (3) one or more MiMe alleles at each of one or more MiMe loci of the component of progression through the second division of meiosis comprising OSD1, CYCA1, TDM1, PC1, PC2, FC, or any combination thereof; or
(b) (2) one or more MiMe alleles at each of one or more MiMe loci of the component of DNA double strand breakage during meiotic recombination comprising PAIR1, SPO11-1, SPO11-2, PRD1, PRD2, DFO, MTOPVIB, DSY1, SY1, SY2, SY3, SY4, or any combination thereof, and (4) one or more MiMe alleles at each of one or more MiMe loci of the component of progression through the first division of meiosis comprise PS1, JASON, or a combination thereof;
wherein each of the of the MiMe alleles comprises a germplasm genetic modification comprising introduction of a premature in-frame stop codon present in the first 30% of the nucleotides of the coding sequence of the MiMe locus following the start codon in the 3′ direction, thereby generating a MiMe allele, and
wherein the genetically modified plant, plant part, or plant cell comprises (i) a complete MiMe genotype comprising only MiMe alleles at each of the one or more MiMe loci, (ii) a partial MiMe genotype comprising at least one MiMe allele and at least one non-MiMe allele at each of the one or more MiMe loci, or (iii) a partially complemented MiMe genotype comprising only MiMe alleles at a first MiMe locus of the one or more MiMe loci and at least one MiMe allele and at least one non-MiMe allele at a second MiMe locus of the one or more MiMe loci.

29. The genetically modified plant, plant part, or plant cell of claim 28, comprising:
(1) a complete MiMe genotype comprising (i) only MiMe alleles at one or more OSD1 loci, wherein each of the MiMe alleles at the one or more OSD1 loci comprise one or more genetic modifications resulting in decreased expression of OSD1, at only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(2) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(3) a complete MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(4) a partial MiMe genotype comprising (i) at least one MiMe allele and at least one non-MiMe allele at one or more OSD1 loci, wherein each of the MiMe alleles at the one or more OSD1 loci comprise one or more genetic modifications resulting in decreased expression of OSD1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(5) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(6) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(7) a complete MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(8) a partial MiMe genotype comprising (i) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(9) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(10) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(11) a complete MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more PAIR1 loci, wherein each of the MiMe alleles at the one or more PAIR1 loci comprise one or more genetic modifications resulting in decreased expression of PAIR1;
(12) a partial MiMe genotype comprising (i) at least one MiMe allele and at least one non-MiMe allele at one or more TDM1 loci, wherein each of the MiMe alleles at the one or more TDM1 loci comprise one or more genetic modifications resulting in decreased expression of TDM1, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1;
(13) a complete MiMe genotype comprising (i) an os allele, wherein the genetically modified plant, plant part, or plant cell is homozygous for the os allele, (ii) only MiMe alleles at one or more REC8 loci, wherein each of the MiMe alleles at the one or more REC8 loci comprise one or more genetic modifications resulting in decreased expression of REC8, and (iii) only MiMe alleles at one or more SPO11-1 loci, wherein each of the MiMe alleles at the one or more SPO11-1 loci comprise one or more genetic modifications resulting in decreased expression of SPO11-1;
(14) a partial MiMe genotype comprising (i) an os allele, wherein the genetically modified plant, plant part, or plant cell is heterozygous for the os allele, (ii) at least one MiMe allele and at least one non-MiMe allele at one or more REC8 loci, wherein each of the MiMe alleles at the one or more REC8 loci comprise one or more genetic modifications resulting in decreased expression of REC8, and (iii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(15) a complete MiMe genotype comprising (i) a ps allele, wherein genetically modified plant, plant part, or plant cell is homozygous for the ps allele, and (ii) only MiMe alleles at one or more SPO11-1 loci, wherein each of the MiMe alleles at the one or more SPO11-1 loci comprise one or more genetic modifications resulting in decreased expression of SPO11-1;
(16) a partial MiMe genotype comprising (i) a ps allele, wherein the genetically modified plant, plant part, or plant cell is heterozygous for the ps allele, and (ii) at least one MiMe allele and at least one non-MiMe allele at one or more SPO11-1 loci, wherein each of the MiMe alleles at the one or more SPO11-1 loci comprise one or more genetic modifications resulting in decreased expression of SPO11-1;
(17) a partially complemented MiMe genotype comprising (i) only MiMe alleles at one or more OSD1 loci, wherein each of the MiMe alleles at the one or more OSD1 loci comprise one or more genetic modifications resulting in decreased expression of OSD1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(18) a partially complemented MiMe genotype comprising (i) only MiMe alleles at one or more CYCA1 loci, wherein each of the MiMe alleles at the one or more CYCA1 loci comprise one or more genetic modifications resulting in decreased expression of CYCA1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(19) a partially complemented MiMe genotype comprising (i) only MiMe alleles at one or more TDM1 loci, wherein each of the MiMe alleles at the one or more TDM1 loci comprise one or more genetic modifications resulting in decreased expression of TDM1, (ii) only MiMe alleles at one or more REC8 loci, wherein each of the MiMe alleles at the one or more REC8 loci comprise one or more genetic modifications resulting in decreased expression of REC8, (iii) at least one MiMe allele and at least one non-MiMe allele at one or more PAIR1 loci, wherein each of the MiMe alleles at the one or more PAIR1 loci comprise one or more genetic modifications resulting in decreased expression of PAIR1, and (iv) at least one MiMe allele and at least one non-MiMe allele at one or more SPO11-1 loci, wherein each of the MiMe alleles at the SPO11-1 locus comprise one or more genetic modifications resulting in decreased expression of SPO11-1;
(20) a partially complemented MiMe genotype comprising (i) a ps allele, wherein the genetically modified plant, plant part, or plant cell is heterozygous for the ps allele, (ii) an os allele, wherein the genetically modified plant, plant part, or plant cell is heterozygous for the os allele, (iii) at least one MiMe allele and at least one non-MiMe allele at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the SPO11-1 locus comprise one or more genetic modifications resulting in decreased expression of SPO11-1; or
(21) a partially complemented MiMe genotype comprising (i) an os allele, wherein the genetically modified plant, plant part, or plant cell is heterozygous for the os allele, (ii) at least one MiMe allele and at least one non-MiMe allele at one or more CYCA1 loci, wherein each of the MiMe alleles at the one or more CYCA1 loci comprise one or more genetic modifications resulting in decreased expression of CYCA1, (iii) only MiMe alleles at one or more REC8 loci, wherein each of the MiMe alleles at the one or more REC8 loci comprise one or more genetic modifications resulting in decreased expression of REC8, and (iv) only MiMe alleles at one or more SPO11-1 loci, wherein each of the MiMe alleles at the one or more SPO11-1 loci comprise one or more genetic modifications resulting in decreased expression of SPO11-1.

30. (canceled)


SEQUENCE LISTING (496 sequences)

SEQ ID NO: 1 - protein, 535 aa, Gossypium barbadense
MFYSRELLARKASLGQIWLLGTRQAKLDRRKANKLDIARLCEEILNPPVPIALRLSSILMGGVVILYEKKVMFLHDDANRFLVEIRNAWKVMNEPTLLPKRKEKAKRKSITLPENDETSMGFTNVGELEEEISMGPGDSFRLADDNDITLFDLFQPNSNIHGQFERFEEADEGTQVNFTLDEHIQIPTTPIPSPSQKEPQRAGGTQERHPEHQLDQQFNKNKEARDQQRQGSVKQRRKPKGIITDEEQTVISNHVYHSWLHDTSDIASRIRRKNSGPASILSTFKIAKLMELPSTVIMDDMLLKGNQEISYPEPLLDLWKKCSQPLHDSPSDLHSGVGSPSHAASIEVQRANVVNKVTPAGINQFVSPGNSGDAVRYTGSSVSGDGVPSGNLEVNIERVGSKKKNLQSTSKNSGSLDTVVEVFHEADTDYQLSRPKRKNLEPDHDFLVETQLTMETPADDPPDMMTENIKKHMKTHFETPGAPQVESLQNLAAGLNKKGAAQLFYRTCVLASQGFLKVQQKVAFGDIFISKGAKM

SEQ ID NO: 2 - protein, 627 aa, Arabidopsis thaliana
MLRLESLIVTVWGPATLLARKAPLGQIWMAATLHAKINRKKLDKLDIIQICEEILNPSVPMALRLSGILMGGVVIVYERKVKLLFDDVNRFLVEINGAWRTKSVPDPTLLPKGKTHARKEAVTLPENEEADFGDFEQTRNVPKFGNYMDFQQTFISMRLDESHVNNNPEPEDLGQQFHQADAENITLFEYHGSFQTNNETYDRFERFDIEGDDETQMNSNPREGAEIPTTLIPSPPRHHDIPEGVNPTSPQRQEQQENRRDGFAEQMEEQNIPDKEEHDRPQPAKKRARKTATSAMDYEQTIIAGHVYQSWLQDTSDILCRGEKRKVRGTIRPDMESFKRANMPPTQLFEKDSSYPPQLYQLWSKNTQVLQTSSSESRHPDLRAEQSPGFVQERMHNHHQTDHHERSDTSSQNLDSPAEILRTVRTGKGASVESMMAGSRASPETINRQAADINVTPFYSGDDVRSMPSTPSARGAASINNIEISSKSRMPNRKRPNSSPRRGLEPVAEERPWEHREYEFEFSMLPEKRFTADKEILFETASTQTQKPVCNQSDEMITDSIKSHLKTHFETPGAPQVESLNKLAVGMDRNAAAKLFFQSCVLATRGVIKVNQAEPYGDILIARGPNM

SEQ ID NO: 3 - protein, 604 aa, Rubus occidentalis
MFYSHSLLARKAPLGEICEEILNPAAPMALRLSSILMGGVVIVYERKVKLLYGTVPVELNEAWKVKSNTDSTVLPKRRAQAKKEAITLPEPEDTDLGGDIDIEQSLDFSHHATLFQQQTGYFLMQLDSVDVGEPLNENAGEGDQHQNFHQADPEDITLPERFDPSDAYGDLFNHRYERFEIEGDDLDFTAGERTQIRAPSPNYIIPSPPSPNYIIPSPPRQDQRQEVQQPHHVFDEMVQMQEVQENAQQRQGPLRGRRRKRRAPVMDNDQTIHPAHVYQSWLQDTSDIVSRRRRNPKPKNIMSTVKIGKLMEQPASVLMGCLFKPGSGETYYPPPLLELWKEKPTHDSPSALTSPPLQPEPSSWSPPERQTYQDSPVEEFHSGGGSQSFATPTEQIRTNLVNNDVPPELMDELRANLNGMRGADTKLATPGNSGDERRSIPSTSSGKGVNKNSSEVNSGRSNRKRRYSSIHSNDGLEPVSEDNGCHHPDPNFNVSRLSGIEQELLMETGPTQTPQPIIDQPLEKMTDDIRKELKSYFEAPGGPEVESLDKLTAGMNRKGAALLFYQTCVLATRNFVKVEQKAPYEDILITRGPNITEVEELVLK

SEQ ID NO: 4 - protein, 527 aa, Citrullus lanatus
MFYSHQLLARKAPLGQIWMAATMHAKINRRKLDNEEILNPSVPMALRLSGILMGGVVIVYERKVKILYEDVTRLLVEINEAWKVKAAPEPTVIPKGKSLAKKEAVTRPEKDPKDVSYNTSPMRFQQTAFFTMRLDSVDEPYNSDKTVEDDPFDIEEDEDTQPNFASVDQTQIPATFKDSPPPQDKYPEGPPDAEVQDQHLESNVNQPAYESTELRQQRTGAMSTMKIATLMDLPPVVLSERLFTDGSREVYYPAPLLELWMRSIRQPPDSPSERNSAPLPPEPSLSSLPERVNFSDHMDFSFEGHQSGVGSQSFGASMEKLMTRRVNDSNQAEIPEELGPNIMDNVMRMTERNQVVTPGNSVNGVRSVSSSASGHGDLSNNLGTNSVRSNKKRPYSSSRQSSGGLEPVAEENPWHHHSDPNFKLARLSENDPDLLEETARTQTQVTQPAIKHPPADKITDSIRTQMKAHFDTPGAASTESLNNLAAGMNRKAAAMLFYQTCVLASSDYLKVNQQVPFGDIFISKGTK

SEQ ID NO: 5 - protein, 617 aa, Prunus avium
MFYSHQLLARKAPLGQIWMAATMHAKINRRKLDKLDIIKICEEILNPSVPMALRLSGILMGGVVIVYERKVKLLYDDVTRLLVEINEAWKAKSVADPTVLPKGKSQARKEAVTLPENEDIDIEGMLNFSNGATTMFQQTSYFAMRLDSVDEQYVNNDAGEEDPPQQFHQAEAENITLPERFDSCQADAYLYSRLERFDIEGDEETQLNFTSGEHTQIHIPPSPPRDEHQKADTIQDDQHPEQQVNQPEECQEFRQKVRMKQVQERQGPIRRKTRRTQASAMDFEQTIIPGHLYQSWLQNSSDIVSRRGRKRKRTDVMATMKIVNLMELPPIVLIGDLFTTGSRDIYYPRPLLDLWMKSTRTPHASPSERTTPPLPPEPSSSTLPGRQQYEDPPFEDYSGVGSQSLETSIDKQRRHSGMTRGNSDPAQVVLEGLTANLENNGLGGTDANPMVTPGNSDDVRSIPSSASGQAIPSEANSGRSNRKRPYSASGHSSVRLEPVFEVNHPDVNFELSRLHEHGPTSEIDFFVETGPTQTQKPINDQPLDKTTDSIRRLLKSHFDTAGAPQVESLDHLTAGRTRKEAAIFFYKTCVLATHDAIKVEQKVAYGEILISRGPKMG

SEQ ID NO: 6 - protein, 624 aa, Vaccinium corymbosum
MFYSHQLLARKAPLGQIWMAATMHAKMNRKKLDKLNIIKICEEILNPSVPMALRLSGILMGGVVIVYKRKVKFLYDDVNRFLVEINEAWKVKSVPDPTLLPKGKSQAKYETVTLPDINQEAELGEIEQSLNYTNTTMRFQETAYFSMRLDNVDEPFINNDPREGPQSEQHHQVDADNITLFERFDSYQTDTNLYSRFERFDIEGDDETQANFTSQERTQIPSSLIPSPPRPAEPRKSDEILDQHPEDQVYHQPDEGKAASQDLQRQGPTRRKARKPAAFAMDYEQTIIPGHMYQSWLQSASDIVSRRGRKRKRFNAMSTMKIGNLMDLPPMVLLCGLFGSGKREVHYPAPLLELWVRSTEPPHDSPSGKTSPPQPPEPSASSLADRARYNDSMGFLYEDVHGGLGSQSFGVSIEKQRTNVNVNEIPTEVLMEDLRNNFVNNGVRVNEANVMVTPGNSGDEVRSIHSSGSGQGFQSHTSEGNSGRSNKKRPNSSSRHSGSGLEPVAEENPWQHPDPNFKLARLSENGLTPENDLLAETGPTQTQQPIMTPRIDQITNSIRMQLKTHFDTLGPSEKESLNQLAFGMNKKRAAHLFYHTCVLASRDFVRVEQHVPYGDILISRGAKM

SEQ ID NO: 7 - protein, 628 aa, Solanum lycopersicum
MFYSHQLLARKAPLGQIWMAATLHSKLNRKKLSKLNIINICEQILNPSVPMALRLSGILMGGVVIVYEHKVKLLYEDVTRLMVQINEAWKVKAATDPTLLPKGKSQAKYAAITLPENREEELPEIEQTLRNPDTVTMMDFEQTSYFTMRLDNADLYDKPNAQEEPAKDLHQVDPDNITLAERFESHHTDMFNHFERFDIEGDEETQMNYTQPEDAQIPSTPVQSPPREQAHQPDEIPDRHPEDQVKQQSDEVKEAFEQRQDQQRPKPARRRTRKATSLAIDHEQTIISGDIYQSWLQSSSDIASRKRKKRKTLSVLPSMKIARMMEMPPIALLEGLFTNGNKEVHYPAPLLKLWMRNTQPPHDSPSGKTSPPFPPEPSYTSPGERMGNLEPPFEYFQGGVGSPAVGISIEKQRANLNNNKIPPEILMEDLRTNLTNMGLHPTETNGVKETDHMATPGSGDEPRSIPSSGSGHGLLSQNSVTDSSRSNKKRPHSSLKHSGNGLQPVAEENPWHDPTLNFKLTRLSELSENGFTQDNEILMETGPTQTQHPFITQPLDMMTDSIRIQLKSHFDTPGSAEAECLNELTLGMTKKQAACLFYQTCVLATRDFVKVEQELPYGNILISRGAKM

SEQ ID NO: 8 - protein, 700 aa, Solanum tuberosum
MFYSHQLLARKAPLGQIWMAATLHSKLNRKKLSKLNIINICEQILNPSVPMALRLSGILMGGVVIVYERKVKLLYEDVTRLMVQINEAWKVKAATDPTLLPKGKSQAKYAAVTLPENREEELPEIEQTLRNPDTVTMMDFEQTSYFTMQVCKFNNRKLTMIKWFDFPSLSFGYSICFAILSDTTWIRVGNISENKIIIDNFYMRVQLNSRMFNVQRLDNADLYDKPNAQKEPAKDLHQVDPDNITLAEHFESHHTDMFNHFERYHDEQFDIEGDEETQMNYTQPEDAQIPSTPVPSPPREQAHQPDEIPDRHPEDQVKQQSDEVEEAFEQRQDQQRPKPARRRARKATGLAIDHEQTIISGDIYQSWLQSSSDIASRKRKKRKTLRVLHSMKIARMMEMPPIALLEGLFTNGNKEVHYPAPLLKLWMRNTQPPHDSPSGKTSPPYPPEPSYTSPGERMGNLEPPFEYFQSGVGSPAVGISIEKQRANLNNNKIPPEILMEDLRTNLTNMGLHPTVANGVKETDHMATPGSGDEPRSIPSSGSGHGLLSQNSVTNSSRSNKKRPHSSSKHSGNGLQPVAEENPWHDPTPNFKLTRLSELSENGFTQDNEILMETGPTQTQHPFITQPLDMMTDSIRMQLKSHFDTPGSAEAECLNELTLGMTKKQAACLFYQTCVLATRDFIKVEQELPYGNILISRGAKM

SEQ ID NO: 9 - protein, 747 aa, synthetic construct
  REGION 1..747: note = Synthetic Construct
  VARIANT 37: note = Xaa = L or I
  VARIANT 46: note = Xaa = N or D
  VARIANT 147: note = Xaa = N or D
  VARIANT 243: note = Xaa = Q or E
  VARIANT 319: note = Xaa = Q or E
  VARIANT 644: note = Xaa = L or I
  VARIANT 1..747: note = Xaa = Any Amino Acid, or no amino acid (except at positions 37, 46, 147, 243, 319 and 644)
MFYSHQLIVTVWGPATLLARKAPLGQIWMAATXHAKXNRKKLDKLXIIXICEEILNPSVPMALRLSGILMGGVVIVYERKVKLLYDDVTRLLVEINEAWKVKSXXDPTLLPKGKSQAKKEAVTLPEINXEXDLGEDIDIEQTLNXXXXXTXMXFQQTXYFXMRLCKFNNRKLTMIKWFDFPSLSFGYSICFAILSDTTWIRVGNISENKIIIDNFYMRVQLNSDXXVXXXXDDXXXXNNXXXXXXPXXXFHQXDXDNITLXERFXSXXTDXXXXXXRFHDERFDIEGDEETQXNFTXXEXTQIPXTSPNXXIPSPPRXXXXXXXPDXXXXDXHPEXQVXQQXFXDEXXEAXXXQXQDQQRQGPXRRRARKXXXXAMDXEQTIIXGHXYQSWLQXXSDIXSRXRRRKRKXXXXMSTMKIAXLMELPPXVLXEXLFTXGXXEVXYPAPLLXLWMRXTQPPHDSPSXXTSPPLPPEPSXXSXXERXXXXDXPFFXXEXXXSGVGSQSXGXSIEKQRANXNXXVXNXXPPEXLMEXLRXNLXNNGXXXTXXNXVKETDHVTPGNSGDEVRSIPSSXSGXGXXSIXNSEXNSKXRSXNKKRPXSSSXHSXXGLEPVAEENPWHHHPDPNFKLTRLSRLSENGFTXDXXXLXETGPTQTQVTXPXIXHQPLDMMTDSIRXQLKSHFDTPGAPEVESLNXLXAGMNXKXAAXLFYQTCVLATRDFXKVEQXVPYGDILISRGAKITEVEELVXM

SEQ ID NO: 10 - protein, 591 aa, Musa acuminata
MFYSHQLLARKAPLGQIWMAATMRAKMNRRKLDKLDIIKICEEILNPSVPMALRLSGILMGGVVIVYERKVKLLYDDVTRFLVEINAAWKVKTVSDPTVLPRAKAQAKFEAVTLPEYVDMEPEQPMMFPDASIATAAFQRMRLDDLEEHYINIDLRDDDLAGNDHQAEPENITLFETFGSGVAETDLYNHFERFDVGDDETHINFTPQEEPQFEATLIPSLPHEDEIRTFTHLHSAVENHQTEEKEEQRANDEDVQRQQPVKRKAHRKPSHRIMDDRQLMIPGNIYQLWLQDTSDIVSKRGRTQCLRSVNPIRSTKISNLMDLPPVALISGLEMFPAKVHYPSPLMELWRKCTEVNISPSGDKSPPAQQREVTETLLEEVQGEIGSNSLDVSIEKLRANLENLDFQGFDDAFSMDHFVTPGSSAGQSSKSMPSSGSGHAFMPLEPEIQLPSVRSKRKQHSSSKSFRNLDPVEEELPLQQDVRGSKIRRLSETGPTPDFELEETGPTQTPVTPPSNPAVDNTTLLIRTHLKLHFDTPGAPQSESLNQLAFGMYKRKAAQLFYQTCVLVTCDFIKVQQHEAYGDISISRGPKM

SEQ ID NO: 11 - protein, 603 aa, Zea mays
MFYSHQLLARKAPLGQIWMAATLHSKINRKRLDKLDIIKICEEILNPSVPMALRLSGILMGGVVIVYERKVKLLYTDVSRLLTEINEAWRIKPVTDPTVLPKGKTQAKYEAVTLPEINMVVEQPMFFSEPDGAKFRRMGLEDLDEQYVQVNLDDDDFSHADDRHQAKAVNITLVDNFESGLAETDLFNHFERFDIADDETTVNITPDEYPQVPSTLIPSPPRQEDIPQQEEPYYAAPSPVHGEPQQGGPEDQEEQKMKQPPKASKRKARWEVPRVIMDNNQMMIPGNIYQTWLKDASSLVSKRRKLNSNFNFIRSTKISDLMHIPPVALISHDNLFSELCYPKPLMQLWKDCTEVKSTKASSGGQRSSSQEPQPKNSPPQAGGEYEMETGGLPMDLTDGIEKLRANMSAKYDRAYNILHSDHSVTPGSPAGLSRRSASSSGGSGSAFIQLDPEVQLPSGSGRSKRGQHSSARSLGNLDTVEEDFPLEQEVRDFKMRRLSDYVPTPDLLEETEPTQTPYERRSNPMDKITETIQSHLKLHFDTPGVPQSESLSHLAHGMTKARAARLFYQIAVLATCDYIKVTQLERKGDELYGDILISRGLKM

SEQ ID NO: 12 - protein, 605 aa, Sorghum bicolor
MFYSHQLLARKQPLGQIWMAATLHSKINRKRLDKLDIIKICEEILNPSVPMALRLSGILMGGVVIVYERKVKLLYSDVSRLLTEINEAWRIKPATDTTVLPKGKAQAKYEAVTLPEKSINMVVEQPMFFSETDGARFRGMRLEDLDEQYFNVNLGDDDFSHADHRHQAEAVNITLVDNFESGFAETDVFNRFERFDIADDETTVNISPDEHPKVPSTLVPSPPRQEDPPQQEEQYAAPFLVREEPQQGGPEEQKMKQTPKASKRKARRELPQVIMDNNQMMIPGNIYQTWLKDASSLVSKRRKVNSNFNFIRSTKISDLMDIPPVSLISHDNSSSELYYPKPLMQLWKDCTEVKSTKVSSGGQRSSSQEQQPRNSPPHEFPPQAGGEYEMETGGLPMDFTDGIEKLRANMSAEYDRAYDTLHSDHSVTPGSPGLSRRSASSSGGSGQAFIPLDAEVQLPSGSGRSKRGRHSSARSLGNLDTVEEDFPLELEVMDVKMRRLSDYAPTPDLLEETEPTQTPYERRSNPMDKVTEAIMSHLKLHFDSPGAPQSESLSHLAHGMTKARAARLFYQTTVLATFDYINVTQLKPHGEELYGDILISRGSKI

SEQ ID NO: 13 - protein, 608 aa, Oryza sativa
MFYSHQLLARKAPLGQIWMAATLHSKINRKRLDKLDIIKICEEILNPSVPMALRLSGILMGGVAIVYERKVKALYDDVSRFLIEINEAWRVKPVADPTVLPKGKTQAKYEAVTLPENIMDMDVEQPMLFSEADTTRFRGMRLEDLDDQYINVNLDDDDFSRAENHHQADAENITLADNFGSGLGETDVFNRFERFDITDDDATFNVTPDGHPQVPSNLVPSPPRQEDSPQQQENHHAASSPLHEEAQQGGASVKNEQEQQKMKGQQPAKSSKRKKRRKDDEVMMDNDQIMIPGNVYQTWLKDPSSLITKRHRINSKVNLIRSIKIRDLMDLPLVSLISSLEKSPLEFYYPKELMQLWKECTEVKSPKAPSSGGQQSSSPEQQQRNLPPQAFPTQPQVDNDREMGFHPVDFADDIEKLRGNTSGEYGRDYDAFHSDHSVTPGSPGLSRRSASSSGGSGRGFTQLDPEVQLPSGRSKRQHSSGKSFGNLDPVEEEFPFEQELRDFKMRRLSDVGPTPDLLEEIEPTQTPYEKKSNPIDQVTQSIHSYLKLHFDTPGASQSESLSQLAHGMTTAKAARLFYQACVLATHDFIKVNQLEPYGDILISRGPKM

SEQ ID NO: 14 - protein, 603 aa, Brachypodium distachyon
MFYSHQLLARKAPLGQIWMAATLHAKINRKRLDKLDIIKICEEILNPSVPMALRLSGILMGGVVIVYERKVKLLYDDVSRLLVDINEAWRIRPVVDHTVLPKGKAQAKYKAVTLPENMMDMEVEQPMLFSDTNTARFRGMSLDDLGEQYFNVNLDDDDFSRAEHHHQAEAVNITLVDNFESGLAETDVFNRFERFDIADDDTTVNITPDEHPQAPSTLAPSPPKEDPPQQQEQHYAAPSPNQEEPQQGDPSKDQEEQKMKERQPNRPSKRKARGKGPQVTMDNQTMIPGNIYQTWLKDPLSLISKRRRVSSKINPIQTIKIGDLMELPPVALISYSEKSPLELYYPKQLMQLWKECTEVKSPKSSSPGGKSPSSQEQQSRNSPPQPQGEYQGEMGAQPMDFTDGIEKIRGNKSGEYERVDDALHGDHSVTPGSPGLSRRSASSSGGSGRGAFVPLDPEIQFYSGGGRSKRRQHSSGRSLGNLDPVEEESPLEQEVKGFKLRRLSDIGPTPELLEETELTQTPYHKQPSPTDQVTESIHSYLKLHFDAPDAPLSESLSQLTYGMTTARAARLFYQTCVLATLDRIKVTQVEPYGAILISRGLNM

SEQ ID NO: 15 - protein, 603 aa, Hordeum vulgare
MFYSHQLLARKAPLGQIWIAATFKSKINRRRLDKLDIIKICEEILNPSVPMALRLSGILMGGVVIVYKKKVKLLYDDVSRLLIELNEAWKIRPVVDHTLLPKGKAQAKYEAVTLPENMMDMELERPVLFTNTDTARFRGMRLEDLDEQYVNVNLDDDDISRADRHHQAEAVNITLVDNFESGFAETDIFTRFERFDIADDDTIFNITPDGHPEAPSTLVPSPPRPEDPFQQQQQRAAPSPIREEPQQGDSLKEQEEQKTKEKQPTKRAKRKARGKGPQVIMDNQIMIPGNIYQSWLKDPSSLTSKRRQVRSKINPIQAIKMGELMDLPPSTLMCCSDDSQEIYYPQQLRQLWKECTKVNPPKPSSSSGVKSSSSSQETQPRNSSPQPQGDQNEMGAQPMDFTDLIEEMRANKSGGFEGVFDGPLGAHSVTPGSPGLSRRSASSSGGSGRGGFLPLGPEIPLQFGDGRAKRKQLSSGRSLGNLDPIEEEFPMEQEGRDFKLRRVSEFGPTPDLMEETEPTQTPFSKQSSPPDHITESIHSYLKLHFESADAPLSESLSHLTHGMNTAQAARLFYQTCVLATRDHIKVTQDEAYGPIHISKGANM

SEQ ID NO: 16 - protein, 608 aa, Triticum aestivum
MFYSHQLLARKAPLGQIWMAATLHAKINRKRLVKLDIIKICEEILNPSVPMALRLSGILMGGVVIVYERKVKLLYDDVSRLLIEINEAWKIRPAVDHTVLPKGKAQAKYEAVTLPENAVDMEVEQPVFFTDTYTTRFRGMRLEDLDEQYVNVNLDDDDISRADRHHQAEAVNITLVDNFESGLAETDIFNRFERFDIADDDTTVHITLDGHPEAPSTLVPSPPRPEDPPQQQEQCAAPSPIREEPQQGDSLKEQEEQKTTEQQPTKRAKRKARGKGPQVIIDNQIMIPGNVYQSWLKDPSSLTSKRRQVRSKINPIKAIKIGELMDLPPSALMSFSNDSQEIYYPQQLMQLWKECTKVKTPKPSSSSGDKSSSSSQEKQPRNPSPQPQGDQNEMGAQPMDFTPMDFTDGIEKMRANKSGEFEGVFDGPHGDPSVTPGSPGLSRRSASSSGGSGRGAFLPLDPEIQLQSGSGRAKRRQLSSGRSLGNLDPVEEEFPMEQEEREFKLRRLSDMEPTPDLMVETEPTQTPFMKQSSPPDHITESIHSYLKLHFESPDAPPSESLSQLTYGMNTAQAARLFYQTCVLATLDRIKVTQVEPYGPILISRGANM

SEQ ID NO: 17 - protein, 608 aa, Aegilops tauschii
MFYSHQLLARKAPLGQIWMAATLHAKINRKRLDKLDIIKICEEILNPSVPMALRLSGILMGGVVIVYERKVKLLYDDVSRLLIEINEAWKIRPAVDHTVLPKGKAQAKYEAVTLPENAMDMEVEQPVLFTDTDTARFRGMRLEDLDEQYVNVNLDDDDISRADRHHQAEAVNITLVDNFESGLAETDIFNRFERFDIADDDTTVHITLDGHPEAPSTLVPSPPRPEDPPQQQEQCAAPSPIHEEPQQGDSLKEQEEQKTTEQQPTKRAKRKARGKGPQVIMDNQIMIPGNVYQSWLKDPSSLISKRRQVRSKINPIKAIKIGELMDLPPSALMSCSDDSQEIYYPQQLMQLWKECTKVKPPKPSSSSGDKSSSSSQEKQPRNSPPQPQGDQNEMGAQPMDFTPMDFTDGIEKMRANKSGEFEGVFDGPHGDPSVTPGSPGLSRRSASSSGGSGRGGFLPLDPEILLQSGSGRAKRRQLSSGRSLGNLDPVEEEFPMEQEGREFKLRRLSDIEPTPDLMVETEPTQTPFTKQSSPPDHITESIHSYLKLHFESPDAPPSESLSQLTYGMNTAQAARLFYQTCVLATLDRIKVTQVEPYGPILISRGANM

SEQ ID NO: 18 - protein, 630 aa, synthetic construct
  REGION 1..630: note = Synthetic Construct
  VARIANT 351: note = Xaa = L or I
  VARIANT 473: note = Xaa = L or I
  VARIANT 1..630: note = Xaa = Any Amino Acid, or no amino acid (except at positions 351 and 473)
MFYSHQLLARKAPLGQIWMAATLHXKINRKRLDKLDIIKICEEILNPSVPMALRLSGILMGGVVIVYERKVKLLYDDVSRLLIEINEAWXIXPVVDHTVLPKGKAQAKYEAVTLPENXMDMEVEQPMLFSDTDIATARFRGMRLEDLDEQYVNVNLDDDDFSRADXHHQAEAVNITLVDNFESGLAETDXFNRFERFDIADDDTTVNITPDXHPQAPSTLVPSPPRXEDPPQQQEQHXAAPSPXXEEPQQGDXLVKKEQEEQKMKEQQPXKXSKRKARGKGPQVIMDNNQIMIPGNIYQTWLKDPSSLXSKRRXVXCLSKINPIRSIKIGDLMDLPPVALISXSXXSXLEXYYPKQLMQLWKECTEVKSPKXSSSXGXKSSSXEQQXQNXPPRNSPPQPQGXYQXEMGAQPMDFTPMDFTDGIEKLRANKSGLEXEXVXDXXHGDHSVTPGSPAGLSRRSASSSGGSGRGAFXPLDPEIQLPSGSGRSKRXQHSSGRSLGNLDPVEEEFPLEQEVRDFKLRRLSDXGPTPDLELEETEPTQTPYXKQSXPAXDXITESIHSYLKLHFDSPXAPQSESLSQLXHGMTTAXAARLFYQTCVLATXDXIKVTQXEPXGXELYGXILISRGXNM

SEQ ID NO: 19 - protein, 461 aa, Citrullus lanatus
MEGNRGSLERIGILRKITELTKSMIVELSNGSSKMISIDRFKSYCTNSLENWFNLSEGKETLTLQRESHVRRLDVLLRVLLIIQQLLRENRHSSKRDIYYMHPSVFTDQSVVDRAINDICILLQCSRHNLNVGDTLQQRSIAVFFKSYSLCLEAASDDPFNLCPLDRLVMGWLRFSEAGRRFDCIKKPNSAHFIPVQVEEVTDIASPADYILVVEKESGEGYSDKSWFHGSRKLNFKFYFDQLKYLVSYFERPVFQRLANDRFCSRNRCIVITGRGYPDVPTRRFLRILVDVLALPAFCLVDCDPYGFDILTTYRFGSMQMAYDAKYLRIPQLQWLGAFASDSEKYNLPEQCLIPLTLQGEGSCIQTHTHTLTLYAFSNVNFGISDILSFLVNLSDKRRTEALLQRCYLRREVPQWRLELDSMLQRGVKFEIEALSVHNISFLSEEYLPSKIKNELTTGKF

SEQ ID NO: 20 - protein, 365 aa, Rubus occidentalis
MEGKRSRWSSQPRPRDLLRKIKGFTRAIVEDLSNGRSPEIPIDRFRNYCSNPSANCYCSSDLPKGKETLTLRRESHVHRLDVLLRVLVVVQQLLQENRHASKRDIYYMHPSIFSDQSVVDRAINDICILLQCSRHNLNVVSVGKGTFQHFSECCKVNNIMAYPVPVNVEEVKDIVSVAQYILVVEKESVFQRLANDQFCNSNRCIVITGRGYPDISTRRFLGLLVDTLHLPIYCLVDCDPYGFDILSTYRFGSLVRPPDQSINLSSTQMAYDAKFLRVPEIIWLGAFPLDSEKYHLPQQCLLPLTAEDKRKTEAMLQRCYLEREVPQWRLELQLMLESGVKFELEALSSLLMGVMGQAMLRKHIH

SEQ ID NO: 21 - protein, 409 aa, Vaccinium corymbosum
MEGTRSNTQPSDLLKKIKDVLLRVLLIVQQLLQENRHGSKRDIYYMHPSVFQGESGFCFGIHFLLRFFSSSLYRGHSLVMGWLRFHEAERKFDCISSLNTAQPVPVHVEDVKDLVSVAHYILVVEKESVFQRLANDQFCNKNRCIVITGRGYPDVPTRRFLRLLMEKLSLPAYCLVDCDPYGFDILMTYRFGSMQMAYDAKFLRLPEIHWLGVFPSDGEKYGIPQQCHLPLTAEGEMDTFEKAFFNHKSRTEAMLLRCYLQREVPHWRSELELLLQRGVKFEIEALSVHSLSFLAEEYIPLKIKGEYSVTIDTEEGLAKVSGEVDPNVLLSAIGKSSKHAELKWVQLKSPTPQNRSHYYHNDDMGKSYGPYSLSYGQDYYGYGQPIEPPHTGRRRALLEAEGRWCDALS

SEQ ID NO: 22 - protein, 362 aa, Arabidopsis thaliana
MEGKFAISESTNLLQRIKDFTQSVVVDLAEGRSPKISINQFRNYCMNPEADCLCSSDKPKGQEIFTLKKEPQTYRIDMLLRVLLIVQQLLQENRHASKRDIYYMHPSAFKAQSIVDRAIGDICILFQCSRYNLNVVSVGNGLVMGWLKFREAGRKFDCLNSLNTAYPVPVLVEEVEDIVSLAEYILVVEKETVFQRLANDMFCKTNRCIVITGRGYPDVSTRRFLRLLMEKLHLPVHCLVDCDPYGFEILATYRFGSMQMAYDIESLRAPDMKWLGAFPSDSEVYSVPKQCLLPLTEEDKKRTEAMLLRCYLKREMPQWRLELETMLKRGVKFEIEALSVHSLSFLSEVYIPSKIRREVSSP

SEQ ID NO: 23 - protein, 313 aa, Gossypium barbadense
MEGIRSSTRSRDLLRKIKEFTLSILEDLSEGLSPLIFIQSFRTYCTNLDSICSCRSNQPSGHEILTFQKESHVHRIDVLLRVLLIVQQLLQENKHASKRDIYYMHPLIFSDQSVVDRAINDICILLQCSRHNLNVVSVGNGLVMGWLRFFEAGRKFDCINSPNTAHTVPVLVEDVKDLVSAALYILVVEKESVFQRLENDKFCSKNRCIVITGRGYPDVPTRRFLRLLVDKLCLPVYCLVDCDPYGFDILATYRFGSMQMAYDAKFLRVPQIQWLGAFPSDSENYDLPKQCLLPLTIEGDTYSKDQWLPPAPS

SEQ ID NO: 24 - protein, 365 aa, Prunus avium
MEGKHSRWSSQPRPCDLLRKIREFTRAIVEDLSNGRSPEILIDRFRNYCTKPDSNCDCSSDLPILKETLTLRRESHLRRLDVLLRVLVIVQQLLQENRHGSKRDIYYMHPSVFSDQSIVDRAINDICILMQCSRHNLNVVSVGKGLVMGWLRFSEAGNMFDCINRPNTAFPVPIHVEEVKDIVSVAKYILVVEKESVFQRLANDQFCNTNHCIVITGRGYPDISTRRFLGLLVDTLHLPTYCLVDCDPYGFDILSTYRFGSMQMAYDAKFLRVPEILWLGGFPLDSEKYLLPQQCLLPLTAQDKRKTEAMLRRCYLQREVPRWRLELQLMLERGVKFELEALSVHELNFLSAVYIPSKIQGGVYI

SEQ ID NO: 25 - protein, 362 aa, Solanum tuberosum
MEGKRSNTQPLTLIRKIKGFTRSIVEDLARGRAPLIYIDRFRNYCTDTSGNCSCSSGLSTGKEAISLKRECHARRLDVLLRVLVIVQQLLQENRHGSKRDIYYMHPTVFKEQSVVDRAINDICILLQCSRHNLNVVSVGNGLVMGWLRYVESGRKINCIGNPHTAYPIPVLVEEVDDIISVAQYVLVVEKESVFQRLANDQFCKRNRCIVITGRGYPDVPTRRFLRLLIDKLHLPVYGLVDCDPYGFDILTTYKFGSLQMAYDATFLRVSEIQWLGVFVQDCDNYSIPQQCLLPLTTEDKRKVEAMLPRCYLQREVPQWRFELELLLHKGVKFEIEALSVHSLTFLSHEYLPSKIHNGGAVF

SEQ ID NO: 26 - protein, 383 aa, Solanum lycopersicum
MEGKRSNTRPLTLLRKIKGFTRSIVEDLARGRPPLIYIERFRNYCTDTSGNCSCSSGLSTGKEAISLKRECHARRLDVMLRVLLIVQQLLQENRHGSKRDIYYMHPTVFKEQSVVDRAINDICILLQCSRHNLNVVSVGNGLVMGWLRYVESGRKIDCISNPNTAYPIPVLVEEVDDIISVAQYVIVVEKESVFQRLANDQFCKRNRCIVITGRGYPDVPTRRFLRLLIDKLHLPVYGLVDCDPYGFDILTTYKFGSLQMAYDATFLRVSEIQWLGVFVQDCDNYSIPQQCLLPLTAEDKRKIEAMLPRCYLQREVPQWRFELELLLHKGVKFEIEALSVHSLTFLSHEYLPSKIHNGGLKILSLNQMILHQNRLKTPISLNS

SEQ ID NO: 27 - protein, 578 aa, synthetic construct
  REGION 1..578: note = Synthetic Construct
  VARIANT 486: note = Xaa = L or I
  VARIANT 386: note = Xaa = Q or E
  VARIANT 401: note = Xaa = N or D
  VARIANT 484: note = Xaa = Q or E
  VARIANT 1..578: note = Xaa = Any Amino Acid, or no amino acid (except at positions 486, 386, 401 and 484)
MEGKRSRWSSXTRPXDLLRKIKXFTRSIVEDLSXGRSPXIXIDRFRNYCTNXXXNCXCSSXLPXGKEXLTLXRESHXRRLDVLLRVLLIVQQLLQENRHGSKRDIYYMHPSVFXDQSVVDRAINDICILLQCSRHNLNVGDXLQVRSIAVFFKSYSXCLEAASDDPFSVGPLNGLVMGWLRFXEAGRKFDCIXXPNTAYPVPVLVEEVKDIVSVAXYILVVEKESGEGYSDKSWFHGSRKLNFKFYFXQLXYVVSYFERPVFQRLANDQFCXXNRCIVITGRGYPDVPTRRFLRLLVDKLHLPVYCLVDCDPYGFDILXTYRFGSMVXPPDQSINLSSTQMAYDAKFLRVPEIQWLGAFPSDSEKYXLPQQCLLPLTAEGEXXXXXXXXHTLTLYAFDNVXFGISDXLSFLVNLXXKRKTEAMLXRCYLQREVPQWRLELELMLXRGVKFEIEALSVHSLXFLSXEYLPSKIXNXXXXXXXXXXXXXXXXXXXXPXXLLSAIGKSXKHAELKWVQLKSPTPQNRSHYYHNDDMGKSYGPYSLSYGQDYYGYGQPIEPPHTGRRRALLEAEGRWCDALS

SEQ ID NO: 28 - protein, 393 aa, Musa acuminata
MNVGRWIDIDAQARPIFLLAAFVPSFAQATTRMAGGRVSSSSPSDLLQRIKGFVRSLVEDLSNDRPPSVALDRYRNYCHDPSGNCTCGDNLPNGKEIISVERESHAYRLLVLFRVLLIIQQLLQENKHGSKRDIYYMHPALFLEQAVVDRAINNICILLKCSRQHLNVVPVGRGLVMGWLRFLEAGRKIYCINSPSTVYPIPVCLEEVVDIVSVARFIIVVEKESVFQRLANDRFCERNHCIVITGRGYPDVPTRRFLCLLVQHLHLPVYCLVDCDPYGFDILMVYRFGSMQMAYDANLLRVPEISWLGVFHSDLQKYHLPDRCLLELTSDDKKKAEAMLLRCYLQKEAPKWRMELEVMLQTGVKFEIESLSFNSLSFLSEYIPTKIHDGSYL

SEQ ID NO: 29 - protein, 383 aa, Zea mays
MAGRDKRRRAAPLEGDEQQLRRRLEEAALLLRRIKGLVRWIVEEVAAGRSPSIVLHRYRNYCSSADSASPSPCACSYDIPVGTDVLSLLHKDYHTSRLNVLLRVLFVVQQLLQQNKHCSKRDIYYMYPSIFVEVAVVDRAINDICILFKCSRHNLNVVPVVKGLVMGWIRFMEGEKKVYCITSVNAAFSIPVDIEAIKDVVSVAHYILVVEKETVFQRLANDKFCERNRCIVITGRGYPDIPTRRFLRYLVELLHLPAYCLVDSDPYGFDILATYKFGSLQLAHDANLLRVPDIRWLGVFTSDFEEYCLPDCCLLRLSPEDRRKAEGILARCYLHREAPEWRSELEAMLQKGVKFEIEALSANSISFLSHEYIPQKIKQGMHL

SEQ ID NO: 30 - protein, 383 aa, Sorghum bicolor
MAGRDKRRRAAPLEGDEQQLRRRQEEAALLLRRIKGLVRWVVEEVAAGRSPSIVLHRYRNYCSAADSASPSPCACSYDVPVGTDVLSLLHNDYHTSRLNVLLRVLLVVQKLLQQNKHCSKRDIYYMYPSIFVEVAVVDRAINDICILLKCSRHNLNVVPVVKGLVMGWIRFVEGEKKVYCITNVNAAFSIPVDIEAIKDVVSVAHYILVVEKETVFQRLANDKFCERNRCIVITGRGYPDIPTRRFLRYLVEQLHLPAYCLVDSDPYGFDILATYKFGSLQLAHDANLLRVPDIRWLGVFTSDFEEYCLPDCCLLHLSPEDRRKAERILARCYLHREVPEWRSELEAMLQKGVKFEIEALSANSISFLSDEYIPQKIKQGMHL

SEQ ID NO: 31 - protein, 381 aa, Oryza sativa
  REGION 1..381: note = SJaponica Group
MAGREKRRRVAALDGEERRRRQEEAATLLHRIRGLVRWVVAEVAAGRSPTVALHRYQNYCSSASAAAASPCACSYDVPVGTDVLSLLHRGSHASRLNVLLRVLLVVQQLLQQNKHCSKRDIYYMYPSIFQEQAVVDRAINDICVLFKCSRHNLNVVPVAKGLVMGWIRFLEGEKEVYCVTNVNAAFSIPVSIEAIKDVVSVADYILIVEKETVFQRLANDKFCERNRCIVITGRGYPDIPTRRFLRYLVEQLHLPVYCLVDADPYGFDILATYKFGSLQLAYDANFLRVPDIRWLGVFTSDFEDYRLPDCCLLHLSSEDRRKAEGILSRCYLHREAPQWRLELEAMLQKGVKFEIEALSACSISFLSEEYIPKKIKQGRHI

SEQ ID NO: 32 - protein, 387 aa, Aegilops tauschii
MAGSGKRRRATVLDDDERRGRRRLEEAALLLHKIKGLVGWVVAEISAGRSPSLALHRYQNYCASADAAAAAASPSTCACSYDAPVGTDVLSLLRKEFHASRLNVLLRVLLVVQQLLQENKHCSKRDIYYMYPSMFVEQAIVDRAINDICILFKCSRHNLNVVPVAKGLVMGWIRFVEGEKKVYCITNVNAAFPIPVSIEAIKDVVSVAHYILVVEKEAVFQRLANDKFCEKNRCIVITGRGYPDIPTRRFLRYLVEQLHLPAYCLVDSDPYGFDILATYKFGSMQLAYDANVLRVPEIRWLGVFTSDFEEYCLPDCCQLQLSSEDRRKLEGILTRCYLHKEAPEWRLKLEAMLEMGVKFEIEALSASSISFLSQEYIPQQIRLGRYI

SEQ ID NO: 33 - protein, 383 aa, Brachypodium distachyon
MEGREKRRRATVLDDDDELRGRRREQEAALLLHKIKGLVGWVVAEVGAGRSPSVALNRYQNYCTSAAASPPTCACSYDAPVGTDILCLLRKEFHASRLSVLLRVLFVVQQLLQQNKHCSKRDIYYMYPSIFIEQAVVDRAINDICILFKCSRHNLNVVPVAKGLVMGWIRFVEGEKKVYCITNVNAAFSIPVSIETIKDIVSVAHYILVVEKETVFQRLANDKFCERNRCIVITGRGYPDVPTRRFLRHLVEQLHLPAYCLVDSDPYGFDILATYKFGSMQLAYDANLLRVPGIRWLGVFTSDFEEYCLPDYCLLDLSSEDRKKLEGILTRCYLHREAPEWRAELEAMLEKGVKFEIEALSASSISFLSQEYIPQKIKLGRHI

SEQ ID NO: 34 - protein, 386 aa, Triticum aestivum
MAGSGKRRRATVLDDDERRGRRRLEEAALLLHKIKGLVGWVVAEISAGRSPSLALHRYQNYCACASAAAAASPSTCACSYDAPVGTDVLSLLRKEFHASRLNVLLRVLLVVQQLLQENKHCSKRDIYYMYPSMFVEQAIVDRAINDICILFKCSRHNLNVVPVAKGLVMGWIRFVEGEKKVYCITNVNTAFPIPVSIEAIKDVVSVAHYILVVEKEAVFQRLANDKFCEKNRCIVITGRGYPDIPTRRFLRYLVEQLHLPAYCLVDSDPYGFDILATYKFGSMQLAYDANILRVPEIRWLGVFTSDFEEYCLPDCCQLQLSSEDRRKLEGILTRCYLHKEAPEWRLKLEAMLEMGVKFEIEALSASSISFLSQEYIPQQIRLGRYI

SEQ ID NO: 35 - protein, 384 aa, Hordeum vulgare
MAGRGKRRRATVLDDDERRGRRRLEESALLLHKIKGLVGWVVAELSAGRSPSLALHRYQNYCASAAAAASPSTCACSYDAPVGTDVLSLLRKEFHASRLNVLLRVLLVVQQLLQENKHCSKRDIYYMYPSMFVEQAIVDRAINDICILFKCSRHNLNVVPVAKGLVMGWIRFVEGEKKVYCITNVNTAFPIPVSIEAIKDVVSVAHYILVVEKEAVFQRLANDKFCKKNRCIVITGRGYPDIPTRRFLRYLVEQLRLPAYCLVDSDPYGFDILATYKFGSMQMAYDANVLRVPEIRWLGVFTSDFEEYCLPDCCQLPLSSEDTRKLEGILTRCYLHREAPEWRLKLEEMLEKGVKFEIEALSSSSISFLSQEYIPQKIKLGRYI

SEQ ID NO: 36 - protein, 454 aa, synthetic construct
  REGION 1..454: note = Synthetic Construct
  VARIANT 454: note = Xaa = L or I
  VARIANT 173: note = Xaa = Q or E
  VARIANT 450: note = Xaa = Q or E
  VARIANT 1..454: note = Xaa = Any Amino Acid, or no amino acid (except at positions 454, 173 and 450)
MNAGRWIDIXKRRRATVLLAAXVPSFADDDERRGRRRLEEAALSDLLHXIKGLVRWVVAEVXAGRSPSXALHRYQNYCXSAXAAXXSPWFSFRARSRDHLLPDQFTVTSLEQFXPKCVTSVDPGRALVRXXCACSYDXPVGTDVLSLLXKEXHASRLNVLLRVLLVVQQLLQXNKHCSKRDIYYMYPSIFVEQAVVDRAINDICILFKCSRHNLNVVPVAKGLVMGWIRFVEGEKKVYCITNVNAAFXIPVSIEAIKDVVSVAHYILVVEKETVFQRLANDKFCERNRCIVITGRGYPDIPTRRFLRYLVEQLHLPAYCLVDSDPYGFDILATYKFGSMQLAYDANLLRVPEIRWLGVFTSDFEEYCLPDCCLLXLSSEDRRKXEGILTRCYLHREAPEWRXELEAMLEKGVKFEIEALSASSISFLSQEYIPQKIKLGRXXXXXXXXXXXATX

SEQ ID NO: 37 - protein, 451 aa, Arabidopsis thaliana
MKMNINKACDLKSISVFPPNLRRRSAEPQASQQLRSQQSQQSFSQGPSSSQRGCGGFSQMTQSSIDELLINDQRFSSQERDLSLKKVSSCLPPINHKREDSQLVASRSSSGLSRRWSSASIGESKLAQISEELEQRFGMMETSLSRFGMMLDSIQSDIMQANRGTKEVFLETERIQQKLTLQDTSLQQLRKEQADSKASLDGGVKFILEEFSKDPNQEKLQKILQMLTTIPEQVETALQKIQREICHTFTREIQVLASLRTPEPRVRVPTAPQVKAKENLPEQRGQAAKVLTSLKMPEPRVQVPAAPQAKENFPEQRGPVAKSNSFCNTTLKTKQPQFPRNPNDASARAVKPYLSPKIQVGCWKTVKPEKSNFKKRATRKPVKSESTRTQFEQCSVVIDSDEEDIDGGFSCLINENTRGTNFEWDAEKETERILRTARRTKRKFGNPIIIN

SEQ ID NO: 38 - protein, 411 aa, Citrullus lanatus
MRLKINKACDLSSISVLPPQSRHARSNALTTGQQAPQLRSQPSQQSFSQGLSSQHGMYSQLSQNSFDEVVTNEQRFSSQDRENSIKKNSFFTPLSYSREDCQMALSKSSTSLTQRWKSTPLPDSKCQVGEELDRRLGTIENSLNKFGKILDSLHSDILQLNKGMKQVSLDVEGIWQKMLVNSSSLQSMNKEHDDMKATLDGGFKLISDLLKKDAHQERLHEISDVLSSLEKQVQASQLKLKNDFISTFTQEMKAIVCTLKNPLQKFPPRSIQSPEGTACLDVPSQKRHRCKIQSASPNVCIRATAVQKEEVGRWKSVKVGKTFCTSSVVKKDDPKKVIPFSEQCLVLEREYRVIVDSDEEIERSFSCLIEEKKTGENVRYYSIEDAKEESARILRKARRQKRKYCNPIIIN

SEQ ID NO: 39 - protein, 451 aa, Solanum tuberosum
MKLKMNKASDLSSISVLPPHARRPSVVPSAAESSVFGKSQPSQVRSQQQSQQSFSQGFSSQQAIYSQLSQSSLDDFVPNEQRLGSQERENSAKRMSCLPHINYTREETQMQLSKPSTNYLRKWSVPESKYQIEELEHKIGMVETSLSRQGMILDSIQSDIMQVNRGTKEILMEMESLRPKLVSHDELLQLMNKEREDLKASLEGTFKSMFEELRENKYQENIRDLLSLLRAMPNKLEMCILQLQTDLSKTLAKEIQIISPRPDCHGKKHETAPVDPPKVINDCSLPREVKFLKKDQKTTTPPKIEMGSWTTVKINQTCMKQKNPIRSHTQNEAFQKLLPLYEMGSWTTVKNNQTCMKQKIPIRSLTQNEAFQQKIPIRSHTQNGAFQQERGHRITIESDEDIDAGFSCLLEENGTENPGIYPLDDAKEESARILRKARRRKRKHFNTIIID

SEQ ID NO: 40 - protein, 441 aa, Solanum lycopersicum
MKLKMNKASDLSSISVLPPHARRPSVVPSAAESSVFGKSQPSQVRSQQQSQQSFSQGFSSQQAVYSQLSQSSLDDFVPNEQRLGSQERENSAKRMSCLPHINYTREETQMQLSKPSTNYLRKWSVPESKYQIEELEHKIGMVETLLSRQGMILDSIQSDIMQVNRGTKEMLMEMESLRPKLVSHDELLQLMNKDREDLKASLEGTFKSMFEELRENKYQENIRDLSSLLRAMPNKLEMCILQLQTDLSKTLAKEIQIIAPRPDFHGQKHETAPVDPPKGINDCIPPREVKLLKKDQKTTTPPKIEMGSWTTVKINQTCMKQKIPIRSHTQNEAFQQKIPIRSHTQNEAFQQTIPIRSHTQNGAFQQKIPIRSHTENGAFQQERGCRITIESDEDIDAGFSCLLEENGTGIYPLDDAKEESARILRKARRRKRKHFNTIIID

SEQ ID NO: 41 - protein, 442 aa, Vaccinium corymbosum
MKLKINKACDLSSISVLPPHTSVVVTALFDSKRRSSLVPNGPESSVFGKRQASQLLSQPSQQSFSQGVSSQQGMFSQLSQNSLDDIVTNNQRISSQERENPVKKLSCLPPISYTLEESQMPVSRSSSNLMRKWNSGSALDHRSQTSEELERRIGMIETSMNRFGMIMDSVQSDIMQVNKGTKEALMEIEDVRQKIIANGNLLQLMDKGQQDIKTNLVGGIKSLTDQVGQDIDGEKLKEISSMISALPEQIDVSLHKMQSELCTKITQEIQAMACSLKIPSQKSQNPPTPAVLLAKDRSCRSVQQQMQPLKDPEMPPKICGKTTLLPKIEVGSWTSVKTEQATRKKQKQKRMSPIEVHKQMMVMQDRECRIPIESDEEIDGGFSCLLEGKETDPVQYLKQATDTFHTPHLGNYSMAEAKEETQRILRKARRRKRRHSNTIILD

SEQ ID NO: 42 - protein, 402 aa, Gossypium barbadense
MKLKINKACDLGSISVFPPHTRRSSLPPSVPQSSQLRSQPSQQSFSQGISSQHALFSQISQSSLDEIVTTDQRFGSQERENTASKFSCLAPTNFTREDSQVPISRSSTNIIRKWNPVSAPEHRCQTSEELEHRLSVIETSLNRFGMILDSVQSDIMQVNKGTKELLLEMEGMRQRSIAEDTSLQLMIKGQEDIKASLDGSMKAVSDQLNNDKYRDKLQQIFLVLSALPEQMEASLIKVRNELCNTFTNEIKAIDCKTLSQKAPVAAAILPKSTGCSVTPRTKPLAVKKQAVPPKIYEQNTVAAEVEKGGWKSVKMKQCATNERASCKENDKRKGVSSVEQETFRILIESDEEIDAGFSCLLNDKQTDDAMNILIEEAKEETERILRKARRRKRKPLNPIIIN

SEQ ID NO: 43 - protein, 407 aa, Prunus avium
MKLKINKACDLSSISVLPQHTRRQNAAPNGPQASQLRSQPSEQSFSQGFSSQHGMFSQLSQNSLSDVLTNDQRFGSQERENSVKKIACLPPISHAREESQMQISRSSTNFMRKWSSASGSDHRWQTSEEFEHRIGMMETSLNKFGMILDSVQSDVMQVNKGMKEVSMEMEAMRQKLTAQDNSLQLMSKGQEDFKASLDGGIKSMSEQLSKNTYQDKLQEMFLVLSALPGKIEIIEASLLKSQNKLHDSFVKEMQKMICSQNQSTSIQKCEISSILSPKGVAASRAIPQGKPQPIKSSAVPPKACVQAVVVPKTETGGWKTVKAEKAVFSDSGFRKVQKPNRFTSIQKERECRVIIESDEEIDGAFSCLLDDKETDVDMAAEVKEETQRILRRARRRKRKLCNTIIIN

SEQ ID NO: 44 - protein, 451 aa, Rubus occidentalis
MKLKINKACDLSSISVLPPHTRRSNSVPHGPQSSQLQLRSQPSEQSFSQGLSSQYGMFSQLSQTSLDALTDQRSQERENSVKKISCLPPISHAREESQMQISRTSNNHMRKWSSVSGSDHRCQISEEFEHRIGMMETSLSKFGMILDSVQSDVMQVKKGTKEVSLEMEAIQQKLMVQDSSLQLMNKGQEDFKTTFDAGIKSMLEQLSKNPNQDKLQEMFLVLSALPEKIEASLLSSQNKLHNSFIKEVQKLLCPIQTCQNQITPIQTCKALSILSPKTAASPAILKAEQKPKRKYVRKCKVPSLPSPKGDAFHAIPQQKPQLINKASERLRNPALDSHSTVPPKVCVQPAVVPTTETGGWKTVKAGNATSIDRVPQKVQKHNRITCVQQEREGRVIIESDEDIDGGFSCLLDEKDTDIKNYMIEEVNQETERILRRARRRKRKCCNPIIIN

SEQ ID NO: 45 - protein, 499 aa, synthetic construct
  REGION 1..499: note = Synthetic Construct
  VARIANT 127: note = Xaa = L or I
  VARIANT 471: note = Xaa = L or I
  VARIANT 236: note = Xaa = N or D
  VARIANT 1..499: note = Xaa = Any Amino Acid, or no amino acid (except at positions 127, 471 and 236)
MKLKINKACDLSSISVLPPHTXVVVTALFDSXRRSSXVPXGPESSVFGKSQXSQLRSQQPSQQSFSQGXSSQQRGCGMFSQLSQSSLDDXVTNDQRFGSQERENSXKKXSSCLPPIXYTREESQMQXSRSSTNXXRKWSSXSXPXXXLXQISEELEHRIGMXETSLXRFGMILDSVQSDIMQVNKGTKEVLXEMEXXRQKLXXXDXSLQLMNKGQEDXKASLDGGXKSMXEQLXKXXYQEKLQEIXXVLSALPEXXEIIEASLLKXQNXLXXTFTKEIQXIXCXXXXXXXXXQXXPXXXXXXXXXXXXXRXXXXXKXXXXKKXXXPPKIXXXXXXXXKXEFXXXXXPXGXWKSVKXXXXXTXXXAFXKXXPXXSXXXVXPXXXPXTXXGQWXXXXXXTQNXAFQQKIPXXXXXXXXXXQQERECRXXIESDEEXIDGGFSCLLEEKXTDXVXYLKQATDTFHTPXXXXYXXXXAKEETXRILRKARRRKRKXXNXIIIN

SEQ ID NO: 46 - protein, 397 aa, Musa acuminata
MKLRINKACDLGSISVLPPRRSGGMSSGGDGLGRSQASQIRSQSQQSFSQGISLSQLSQSSFEEPLVNDQRFGSQEKDNSLRKISSLAPIALTREESQLQLTRASSNVMHRWNAASMSDNRFQVSEELEHKLRHIESSINRVGMILDSVQSDVMQVNRAAKELSLEVEGIRQKVSLLENSMQQMVKWEDDIKAFIGGSLTSISDQLIKISSSGKVNEIASAVATLQEQMFSRLARLECEVCRFFSEKEVRESGIKSSNNQHSVKFQSPMSGNYMDIKESLEGNIEEKKVSAAPLMSNKQRSPPFKKEGKLESFKSKLTEPKHTVKHKHIIPHTKQEEFVRVIVDLDEESDGGISCLIVNKGTGKKSSFMEEVQEDTLRILRKARMKKRRQMNSITVV

SEQ ID NO: 47 - protein, 634 aa, Triticum aestivum
MKLKINKACDLASISVLPPRRTRGSSGGGGGGGTSAPAAASAAQQQRSQSMSQKSFSQGIGASFSQGIGSGGASFSQGSGGGGAAFSQGGGGGGAAFSQGGGGGGAAFSQGGGGGGASFSQGGGSASLLHAQSQLSQASLDESLLSLLSQAPARDQRFAMQEDPSKRMSSYLASSASSCMRDESQLQLAKIPTNPIHRWSPSFLDSRCQVNEDVERKFLHLASSVQKMGMILDSVQNDVMQLNRGMKEASLDSGSIQKKFVLLEDSLKQILKGQDDLKALLKGSTKSNPDQLGILTRKLDEISSILSVLPKQVQTEFEQFKGDIFRFFTKEMEGVVRAVRSLDSRLPGVMQMLADQSCTTNAKPLMNHTAAENESPLMNQTQVAEGSPLMNQGPVADRRPQKKPTAVANGRPKRKNQTAVANGRPQRKNQAAGANWRPHRKKPTEIAANSRPQRHLTRVANGRPQVKQTEAADGRSQTNQKPEANGKALIPVTKPIPVPVVYHRKAVDLKPRVEQWMVPKLAGPSYSRPAPPKEQELAIQKVNTEAPINKMQATLGILIDSDDDGDYEGADSDWSASCVILKLEAGCPGDEVVAEEATSEGEGEALEILRRARKRRRREANANANATVLVQDQR

SEQ ID NO: 48 - protein, 670 aa, Hordeum vulgare
MKLTINKACDIASISVLPPRRTGGSSGGGGRMSAAAAQQQQQQRSQSMSQKSFSQGGGGVGSFSQSVGASFSQGMGSGGASFSQGRGASFSQSSSTGGAAAAFSQGGGGGGGGASFSQGRGASFSQGSGTGGAVAAFSQGGGAGSGGADFSQGRGGGGAAFSQGGGGGGASFSQGGGSGSLLHAQSQLSEASLNENLLSQAPARDQRFSLQEDSSKRTPSYPASSASCVREDSQLQLTTIPTNPIHRWSPSLPDSRCQINEEVERKFQHLASSVHKLGMILDSVQNDVMQLNRSMKEASLDSGSIQKKFVLLEDSLKQILKGQDDIKALFEGSTESNPDQLSVLNSHTRKLDEMTSILSALPNHLQAEFGQLKGDTYRIFTKEMEGIARGIRSINTRLDAMQMLAEQSCTTNAKTLMNQTAVAHGSPLMNQTPVAVGSTLMNQGAVADGSPLMNHQGPVADGRPQKRQRPAANGRSQRKKQTPVASGRAQRKRQRPVANGRPQVKQAQTADGRSQMNQKPEANGEALMKQVLPATQVAPAPLVYQRKKEGVQPKAEQGMSKAGTGAQKLAGSGDSRPASPKEQELAIQKDNAEAPINKAMLAILIDSDEEEEEGGGSEGNASCVLLKSTEAGGGVAMAEEAMGEGEALEILRRARKRRRREEANAVVLDH

SEQ ID NO: 49 - protein, 362 aa, Aegilops tauschii
MAMSGINSSIPQTDRLVDWLSAWKLGIIVRATYLSTLVPSPIVTSICHLVGDQGFGLHDDSSKRMPPFPPSSASRAHEEPQQQQPVIVPSSTTLRWNPSPTDTRCRVNEDAERRFQHLSGSVHKVGMVLDSVQTDVIQLNRTMKDASLKSGVVQQKFDLLEDTLQKIIKGQNDLKVLAENSTKSNLDQLNVINSRTSKLNEMSLVVSVCPKQVQADLRELHGDIFRVLTKDMEGVVRDIRSLSTKPAVMPMLPDHVVAKGSPLMNKMSVANERPTMNQTPVAKGSPLMNKMSVANERPTMNQTPVANGRPLMNQTPVANGSPMIINQARVGNGRSQLKQTPVTDAMPQRDLTPEINGRPQKD

SEQ ID NO: 50 - protein, 522 aa, Brachypodium distachyon
MPSQSQLLEASLGQTLLSQQLPAPAHDQIFGLHNVSSQIPSFPPNSASCVQEESQQQLAKISNNTIPWRNPCLAESRCQVANEDAEHRFQQLANSVCRIEMVLDSVQNDVIQLNRSMKEASLDSDSIQRKVVLLDNSLQKILMVQDGEAGSIQQNDVLLDNSLQQILKGQDGLKELLEGGTKSNPDQLSVLNSHTSTLSEISSILSVWQEEIRADLRQLHGDIFRIFTKEMKGVVRAIRSLNSRPAALQMLEDRSCYPNERTWSSQVQVADGSPLMNQIPLANERLKMNQTPVSIGEPLMNLAVVANGSPLINQSPVANGRSQMNQTPVANGRPQMDRTPVANGRPHMEQITTNLFPARSSCSTKAADPKPNIEQGDVKATTPKLFGSSYMLAPKQGEVLNRKVNQQEPTKKAPVTIMIDSDDDSEGHASWVILNTEPADLMKEASREEGMQLLWSARKRSRRKRETRCIDATALPDVMRGKKRMGKAAPEAQEEEAVHVEAIPAKRTCRPNPKYDAEQWTA

SEQ ID NO: 51 - protein, 656 aa, Sorghum bicolor
MAGRVGEYNCPRQQRIQNRSASHSITPLLLGCSSGTGDHHCRRRLLAPARSREQAMKLKINKACDLGSISVLPPRRTGGSGGAGEAGSSAAAVAAGSQQRSQPMSHSQQSFSQGVMGSGGASSLLHSQSQLSQASLDENLLSLHLASPTRDQRFGLHDDSSKKMPSLPVSSASCVREESQLQLAKTPSNPVHRWNPSLPDGRCVVPTEDVERKFQHMASSVHKVGMVLDSVQNDVMQLNRAMKEAALDSGSMQQKIVVLDTSLQKNLKGQDDLKALVESNTKSISDQLTVLNSHSNKLDEISSTLSILPKQIETDLKQQHSDIFRIFRKDMEEIVRAVRSLNSKIDAIQMPTDQRCTINGRPLMNQLPVDRNERPQVNQTPEVTRMSQTPVATMVNQTSVASLVNQTPAANGRHLVSQIPAANGKTLVSQTSVANGRSLMSQVPTANGKPLTNQASLANGGSLMSQVPAANGKPLTNQIPAPKGRPMMRQKTGERGRPQMNQIPVASGWTHTNKIPAPEVHPAPLVFPASAKAAADPKAKVDDGKLKVLPQKLTGSRSRVTPKQEEAASTKFSRAAATEKVVIFIEDSDDDSDVRASSCVILRSSGSGAGAGAGERECDLMKVGAEESQEIMRRARKRRRREMQATVASMPPEVGH

SEQ ID NO: 52 - protein, 562 aa, Zea mays
MKLKINKACDLGSISVLPPRRTGGSGGAGGAGSSAAPVAAGSQQRSQPMSQHSFSQGVVGSGGASSLLHSQSQLSQASLDENLLTLHLASPARDQRFGFHDDLSKKMTSLPVSSASCVREESQLQLAKTSSNPVHRWNPSLPDGRSVGPTEDVERKFQHMASSVHKVGMVLDSVQTDVMQLNRAMKEATLDSGSIQQKIVLLDALLQKNLKGQDDLKALVESNTKSISDQLTVLNSYSNKLDEISSTLSILPKQIETNLKQQQSDTFRIFRKDMEEIVRAVRSLNNKIDAIQMPTDQSCTTNGRPLMNQLPVDRNERPQVNQTPEVTRVSQTPVATMANQRPVAKGRHLVSQTPAANGKPLTNQTSVANGRSLMSQVPAANGKPLTNQIAVPKGRPMMRQKTAEGGRPQMNQIPVADGWPHTNKIHAPEVRHPVPLVCPAAGPRPKVEEGKLRAVPQKLTGCRSRVTPKQEEAATNTKVVVGRAAATEKVVIFIDDDSDDGGSEALASCVILRPAPLGSGAGECDLVKVGAEESQEILRRARKRRRREMQAIVACMPPELGY

SEQ ID NO: 53 - protein, 609 aa, Zea mays
MTGAGMKLKINKACDLSSISVLPPRRTGGSGGAGSSAAAAAVAAGSQQRSQPLSQQSFSQGAVGSGGASSLLHSQSQLSQASLDENLLTLHLASPARDQGFRLHDDSSKKMTSLPVTSASCVREESQLQLAKTSSNPVHRWNPSLPDSRYFIELHSFLFECLTGSGLTGVVPAEDVERKFQHMASSVHKVGMVLDSVQNDTMQLNRAMKEAALDSGSIQQKIVVLDTSMQKNLKGQGDLKVLVESNTKNIADQLAVLNSHSNKLVEISSALSVLPKQIERDLKQQQSDIFRIFRNNMEEIARAVKSLNSKIDAIQMPTEQRCTTNGRPLMNQLPVDRNERPQVNQTPEVTRVSQTTVASLVNQAPAANGRHLMSKTPAANGKSVMRQTPAENGRHLVSQTPAANGKTLKRQTSVTNGISLMSQVPAANGKPVVIQIPAPKGRPMMRQKTAESGRTEMNEIPVASGRPHTNKIPIAEVHPAPLVCPAKQVATDPKPKVDEEKLKALPQKLTSSRSRVAPKQEEAANTKVISRAAATEKVVIFIDDSDDDNDVRASCVILRPSGSGSLGERECDLMKVGAEESQEIPRRARKRRRREMQATVGVACMPLPA

SEQ ID NO: 54 - protein, 492 aa, Oryza sativa
  REGION 1..492: note = Japonica Group
MKLKMNKACDIASISVLPPRRTGGSSGASASGSVAVAVASQPRSQPLSQSQQSFSQGASASLLHSQSQFSQVSLDDNLLTLLPSPTRDQRFGLHDDSSKRMSSLPASSASCAREESQLQLAKLPSNPVHRWNPSIADTRSGQVTNEDVERKFQHLASSVHKMGMVVDSVQSDVMQLNRAMKEASLDSGSIRQKIAVLESSLQQILKGQDDLKALFGSSTKHNPDQTSVLNSLGSKLNEISSTLATLQTQMQARQLQGDQTTVLNSNASKSNEISSTLATLQTQMQADIRQLRCDVFRVFTKEMEGVVRAIRSVNSRPAAMQMMADQSYQVPVSNGWTQINQTPVAAGRSPMNRAPVAAGRSRMNQLPETKVLSAHLVYPAKVTDLKPKVEQGKVKAAPQKPFASSYYRVAPKQEEVAIRKVNIQVPAKKAPVSIIIESDDDSEGRASCVILKTETGSKEWKVTKQGTEEGLEILRRARKRRRREMQSIVLAS

SEQ ID NO: 55 - protein, 872 aa, synthetic construct
  REGION 1..872: note = Synthetic Construct
  VARIANT 507: note = Xaa = L or I
  VARIANT 475: note = Xaa = Q or E
  VARIANT 748: note = Xaa = Q or E
  VARIANT 868: note = Xaa = Q or E
  VARIANT 461: note = Xaa = N or D
  VARIANT 574: note = Xaa = N or D
  VARIANT 1..872: note = Xaa = Any Amino Acid, or no amino acid (except at positions 507, 475, 748, 868, 461 and 574)
MXGRVGEYNCPRQQRIQNRSASHSITPLLLGCSSGTGDHHCRRRLLAPARSREXXMKLKINKACDLXSISVLPPRRTGGSSGAGGGXXXSXAAAVAAGSQQRSQPMSQXSXSQGXGXXXSXXXXXGASFSQGMGSGGASFSQGRGASFSQSXSTGGAAAAFSQGGGGGGGGASFSQGRGASFSQGSGXGGAXXXXSXGGGXGSXGAXFSQGXGGGGAAFSQGXXGGGXSFSQGGGXSSLLHSQSQLSQASLDENLLSLHLASPARDQRFGLHDKDSSKRMXSXPXSSASSCVREESQLQLAKXPSNPXHRWNXPSLXDXRSFXELHSXLFECLTGSGLCGVXXNEDVERKFQHLASSVHKVGMVLDSVQNDVMQLNRAMKEASLDSGSIQQKXVVLXLSLQKILMVXDLEAGSIQQNDXLLEXSLQQILKGQDDLKALXEXSTKSNXDQLXVLNSHXXKLXEISSXLSXLPKQXXXDLXQLXGDIFRIFTKXMEXIVRAIRSLNXXXXAXXQMXXDQSCTTNGRPLMNQLPVDRNERPQVNQTPEVTRMSXTPVATMVXQTXVASLVNQTPVAXGSPLMNQXPXANXXXXMNQTXVANGRXLXSXQTPVAXGXPLXXNQXXXANGXXXXXQVPXXAANGXPXXNQTPVAXGRPXMXXQXXXXXGRSQMNQIPVAXGWPHTXKXXPAXXVXPAPLVXPXKXKXAADPKPKVEQGKXKAXPGAQKLXGSXXXRPVXPKQEEXAXXKVNXXXPAXKXEKVVXIIIDSDDDSDXEGGGSEXXASCVILXXXXXGXGXEXGXXEXXXXKXGAEEXLEILRRARXIDXTALPDVMXXKRRMGKAAPERREMQAXVXXXXPXKRTXXPNPKYDAXXXXX

SEQ ID NO: 56 - protein, 262 aa, Prunus avium
MAEARDRLSRPPVDLADTYAQWLSRNRSIYIDPPEQVLQVVPDLSRTPARQRAPTALGFGATGVVGGGGLLRSSLRTSRTGNARGRIPFGFTTPARENTPPAGSSRRRRGRSSNSVLPSWYPRTPLQDISAVVRAIERRRARLGENDGQHTEGQVPQGRNILDQYLLVPGAQLEHGVPVTPYSAVRTKHCPPPSVGRVQKIIREATNTQTAEGEFLTPQKKLLNSIDTVEKVVMEELQRLKRTPSAKKAEREKRVRTLMSMR

SEQ ID NO: 57 - protein, 250 aa, Rubus occidentalis
MPAESRFRLERPFDIAEAFSQRRSGSLAVLIDERGELLESPARSRALATPVHQRRLGFWATGGGVGHGLARSRLRTPRAAIGHGQENMPPVGSSNRRRGRVSNSVLPSWYPRTPLRDITAITRAIERRRARLGEENGETSRGQVPSQQVQDASPEHEVPATTPSTIKKRPCPPPSVRKVHKILLEVAYQPDDGEFLTPQKKLLNSIDKVENVVMEELQRLKRTPSAKKAEREKRHKADGSLRYGGYALVT

SEQ ID NO: 58 - protein, 243 aa, Arabidopsis thaliana
MPEARDRTERPVDYSTIFANRRRHGILLDEPDSRLSLIESPVNPDIGSIGGTGGLVRGNFTTWRPGNGRGGHTPFRLPQGRENMPIVTARRGRGGGLLPSWYPRTPLRDITHIVRAIERRRGAGTGGDDGRVIEIPTHRQVGVLESPVPLSGEHKCSMVTPGPSVGFKRSCPPSTAKVQKMLLDITKEIAEEEAGFITPEKKLLNSIDKVEKIVMAEIQKLKSTPQAKREEREKRVRTLMTMR

SEQ ID NO: 59 - protein, 257 aa, Gossypium barbadense
MAESRDRLGRAVDIAEVFARRRSGPLGILSDESMDLLGSPVRQSVTRRSMGVVGVTTSTARGVGLRRGGSFGTPRIGIRRSRNLYRSTGRENASVTPSPLGRGRGRGTGSVLPSWYPRTPLRDITAVVRAIERRRARLGDGEGQIVETPILPHDETILGSNMSSVSQLEHNFSTPASTTRLKPCPPSVRNVSKILLNVTNQKHEESSEELLTPQKKLLNSIDTVEKAVIEELNKMKKTPSAKKAERQHKVRTLMSMR

SEQ ID NO: 60 - protein, 249 aa, Citrullus lanatus
MSEARDRLERQVDHAEVFARRRSEGILDEQEMNSHLIGTPIARATTTPTAQQRPTNPGPGGGGSNPRRTFGSPISGGIGRNRFLYRSPVFNRENTAASSSRRNRSRGRNSVLPIWYPRTPLRDITAVVRAIERTRARLRENEGQGSDNSPAPSDERALEYSVSVAGDHQEPSISLVTPKPTVGKVPKIILGIANQNVVGAETLTPQKKLLNSIDKVEKVVMEELQKLKKTPSAKKAEREKRVRTLMSFR

SEQ ID NO: 61 - protein, 255 aa, Vaccinium corymbosum
MPESRDRLSRPDEVRATFARQRQSIGGIAILLDEEQPEVGTPFRWGATAMTGTRGPMGVASTRGGRGVRRGSLGTPRSSTRNRRGQENMASGSFRRGRGRGRSTNSVLPSWYPRTPLRDVTYVVRAIERRRAASLRESEGLQIGSPMPQDQNVLVPSEPTSGAQLEHNLSLVTPNPTVRMKKPCQPSVGKVPKILLDITHQTAAGSDCLTPQKKLLNSIDTVEKVVMEELRKLKSTPSAKKAERQNRVRTLMSMR

SEQ ID NO: 62 - protein, 255 aa, Solanum lycopersicum
MAEGRDRLSRQEDPIDIYSRRRRSMGRGGIEIFEDESPESSSRAPIQTTAEAGRMAGTSGGRGGIGRIGFGSPRNRRGRNLFRTPARVIGRQNISPTGQGRNRGRHSVLPAWYPRTPLRDITSIVRAIERTRARLRESEGEQLESVVPQDHTDLGPSESTSGAQLEHTNSLITPRPKTRSRYHTRSVGKVPKILLDITNQSTSEDAECLTPQRKLLNSIDTVEKHVMEELHKLKRTPSARKQERDKRVKTLMSMR

SEQ ID NO: 63 - protein, 255 aa, Solanum tuberosum
MAEGRDRLSRQEDPIDIYSRRRRSMGRGGIEIFEDESPESSSRAPIQTTAETGRMAGTSGGRGGIGRIGFGSPRNRRGRNSLRTPTRVIGRQNISPAGQGRNRGRHSVLPAWYPRTPLRDITSIVRAIERTRARLRESEGEQLESVVPQDHTDLGPSESTSGAQLEHTNSLITPRPKTRSRYHTRSVGKVPKILLDITNQSTSEDSECLTPQRKLLNSIDTVEKDVMEELHKLKRTPGARKEERDKRVKTLMSMR

SEQ ID NO: 64 - protein, 278 aa, synthetic construct
  REGION 1..278: note = Synthetic Construct
  VARIANT 57: note = Xaa = Q or E
  VARIANT 71: note = Xaa = L or I
  VARIANT 153: note = Xaa = Q or E
  VARIANT 1..278: note = Xaa = Any Amino Acid, or no amino acid (except at positions 57, 71 and 153)
MPAEXRDRLSRPPVDXAXXFARRRRSXXGXXXXXXXXXXXLXXXPXRXXXXXXTXXXXGXXXGXXGGGGGXXRXXFGTPRXGIXRGRNRXXXXXXXXGRENXXPXGSSRRGRGRGXNSVLPSWYPRTPLRDITAXVRAIERRRXARLXEXEGXXXEXSXVPQDXXXLGPSESXSGAQLEHXXSLVTPXPXXRXKXXXPPSVGKVPKILLDITNQXXXESXXEXLTPQKKLLNSIDTVEKVVMEELQKLKRTPSAKKAEREKRVRTLMSMRYGGYALVT

SEQ ID NO: 65 - protein, 202 aa, Musa acuminata
MAETRQGVNRGRETTGSATNLGFWIRRAAYLASRTGSRSTRVQSVVFATDDKENVTPSRAARRRGRTRKSPLPEWYPRTPLRDITVIVNALERRRMRVRAAATARQRTSDPEPAAVEKGLLDSSSIPAAAGSSSVSATEQPPQICSSSNASSPREDPPDQPTEYEKNLEIYIGEMERLVTENLKRSPMPPAKRAKRTLISMR

SEQ ID NO: 66 - protein, 232 aa, Brachypodium distachyon
MVEVRTVSRTAVAERSGGGFFIRRVESPGAVVEKGAAKRLARRPLTPSSNKENVPPAWAVATTQKRRSTLPEWYPRSPLRDITSIIKAVERKNLLRDAAARQQLQWTEDSSEPENPAQADQDVHRRTPPTNGTLAAAAVASDPAGSAQAVASTSATCVAEGTLKAATGDCSLQTPSRQGNHPALSDLLEKLLASSIEQIEKMVCQNLTLGDPKAAQPSKTQAVQRRTLMSMR

SEQ ID NO: 67 - protein, 238 aa, Hordeum vulgare
MAEVRIASRTAAADRSGGGGGFFVRRVVSPGAVVDKGAVKPLARLVPSPSSNKENVPPAWAAWTAPKRRSFLPEWYPRTPLRDITSIIKAVERKSRLQDAAARHQLQWDDEEDSSEPDDEEYSSEPENPAQTDEGIHRSTAPTNETLCVGSAQVVETTAASATCLGEGKCATVVKASDDCSLQSPSRPSGEDDVEKQLANSIHEIEKMVSRNLKRTDPKATRTSKTPAVQRRTLMSMR

SEQ ID NO: 68 - protein, 226 aa, Triticum aestivum
MAEVRIASRTAVADRSGGGFFVRRVASPGAVVDKGAVKPLARRVPSPSSNKENVPPAWAAWTAPKRRSSLPEWYPRTPLRDITSVIKAVERKSRLRDAVARQQLQWDEEDSSEPEDPAQTDEGIHGSTAPTNETLGVGSAQVVETTAASATCLGEGESTTVVKASDDCSLQSPSRPSGEDDVEKQLANSIQEIENMVSRNLKRTDPKATRPSKTPAVQRRTLMSMR

SEQ ID NO: 69 - protein, 226 aa, Aegilops tauschii
MAEVRMASRAAVADRSGGGFFVRRVASPGAVVDKGAVKPLARRVPSPSSNKENVPPAWAAWAAPKRRSSLPEWYPRTPLRDITSVIKAVERKSRLRDAAARQQLQWDEEDSSEPEDPAQTDEGIHGSTAPTNETLGVGWAKVVETTAASATCLGEGESTTVVKASDDCSLQSTSRPSGEVGVEKQLASSIDAIEKMVSRNLKRTDPEATRPSKTPAVQRRTLMSMR

SEQ ID NO: 70 - protein, 225 aa, Sorghum bicolor
MPQLRTASRPVLARNSTGGIFIRRRVASPGGAVKPLARRVRTHFSNKENVPPVGAARAKPKRRSPLPDWYPRSPLRDITSIVKALEKRNRLEEDAARQHIQWNEDSPQPVDPTTTVHAEHSDPDSQSTQTQETLGVVASPGSTSAVANNVTSVAEDKQEASSSPSDCLQMAPSKPNDPSPADLEKKMSSSIEQIEKMVRRHMKETPKAAQPSKLVVQRRILMSMR

SEQ ID NO: 71 - protein, 216 aa, Oryza sativa
MPEMRDSKRTALGELSGGGGFFIRRVASPGALAARGPGKPLARRFIRPSNNKENVPPVWAVKATATKRRSPLPDWYPRTPLRDITAIAKAIQRSRLRIAAAQQRSQTPEQNTPHCTEVRDSLDVEPGINSTQIVATPASSLAKDSLKIFSSPSETSLVTPSKPMDPVLLDDMEKKLSSSIEQIEKMVRRNLKRTPKAAAAQPSKRAIQRRTLMSMR

SEQ ID NO: 72 - protein, 227 aa, Zea mays
MPESRDGRSEDLADLSGGVGGGGFFIRRVASPGALAVRGVRKPLARRYISPSRNKENLLPVWALRVTPTKRSPLPGWYPRTPLRDITAIAKAIQRSRSRIAAAQQRSQRIEQSSQSVNVTTPAQAEQDAHIAEASHAVASGSGSTEREAVANPATVLADDNLNVSSLAAEGSLNTPPKPMDPALADKKLSGSIEKVEKLVRKNLKRTSRAAQASRRATQRRNLMSMR

SEQ ID NO: 73 - protein, 224 aa, Zea mays
MRNFKMLEVRTARRPALADISGGGFFMRTVESPGAVLVNGAVKRPARQFLSPSSNKENVPPVGAFRATPKRRTPLPDWYPRTPLRDITSIVKAIERRRSRLQNAAAQQQIQWTEDPSRSVDPITPVQAEQGGVPTTVDGQGVGSPATCLEDGKLKTSSYPSSDCSLQATPSKPNDPALADLVEKKLSSSIEQIEKMVRRNLKRTSKAAQPSKRTIQRRVLMSMR

SEQ ID NO: 74 - protein, 225 aa, Zea mays
MSYTNFSQDYKMPQLRTASRPALASNSAGGFFIRRRVASPGTSQAKGAAKPLARRVRTPAARAKPKRRSPLPDWYPRVPLRDITSIVKALEKRNRLEEDAARQHIQSNEDSSQPVDPTTAEHSNPDSQSTQTQETPGAVASGPSSTSAVANRVTSVAEGKQEATDCSLQVAPSKPNDPSPADLVKKLSGSIEQIEKMVRRHMKETHPKAAQPSKVVVQRRILMSM

SEQ ID NO: 75 - protein, 265 aa, synthetic construct
  REGION 1..265: note = Synthetic Construct
  VARIANT 9: note = Xaa = N or D
  VARIANT 122: note = Xaa = L or I
  VARIANT 150: note = Xaa = Q or E
  VARIANT 1..265: note = Xaa = Any Amino Acid, or no amino acid (except at positions 9, 122 and 150)
MSYTNFSXXXKMXEVRXASRXALADXSGGXXGGGFFIRRRVASPGAVXXKGAVKPLARRVXSPSSNKENVPPXWAARAXPXKRRSPLPEWYPRTPLRDITSIXKAXERRRSRLRXAAARQQXQWXEXSXSSEPXDXEYSSEPENPAQTDXXXHSXPDXSXXXTXETLGXXVXXXXXXXXVAXSATCLAEGXXXXXSXASDDCSLQXPSXKPNDPSXXDXVEKKLSSSIEQIEKMVRRNLKRTPDPKAAQPSKXPAVQRRTLMSMR

SEQ ID NO: 76 - protein, 453 aa, Zea mays
MRRCAEAIARAVVEFLDAVILAFIRSCFGARPRRGSGLRVRPTPAPPRSPSLSAHLAAVSWSSSFQDALVRGDRAAEVIWDEEGLVRDGRFHEDLADGCGIDEELRREASYLKLCGTISETPAELRNEQSYENNLETTNECDSKLTYGPPANCKLLFEANSSQGQRNPRCEERHSLRSILNSEDAGRHHGAEYVPRSASSEKRLFQNMQHKPLDSGGSPFPTPLVLRDDMQTPGTAYASHRGTSISGKRVRTRKQFIYPVLRSIENRLQQIELTEDSSPLASSDPLKERDLGADSTKDPTQASSTSVVKSGLSETPSYSAPDPNASYGVEESLSKSNSDEKNAALSLSRWLKSSSADAENQDDEDCSFLTEKPVFMVAPDLNLDTSNPTPRLPKAWDGNGIPNTTTRYREDQRVSWHTTPFEQRLLKVLSDEDRCPPRKVVRGKLFHLEETAE

SEQ ID NO: 77 - protein, 432 aa, Zea mays
MMGCLFGCFRASGDGGDAKGAGDRNTSLAPATTTTIQQDGTGRRIRPPSRNALSAVFQREDEGSRAAQAASSWAEQTGESKRMDQELEPQTIIQKNCGALLQTTNNIQSVLRDADSVHQKETHSGCLPVMSDDVHFMEAPKVENCETPPRSHQSSTVPDATSSSKTNDELRTSATSLKNNVEESTNENDTEACVQGEEQQQALDLAEDFEECGVSKEDFFPPEKLEDPKCAKNDHVVSMEISISDECSLFQSSEGSVSPSNKIRGSMNTTSTEKSPKTEATIHATRKKLLKSNTSELELPSLAQWLKPPNPKKAFRDEAVTGDRSHSAKSSDEDRPIIGMVAAHWKDKEPANFTSKWWDGNGIPNSTNKYKEDQKVSWHATPFEERLEKALSEEKSLSERNCSSGKTSQFLGVEGEESDTAGSNRLYATAYA

SEQ ID NO: 78 - protein, 509 aa, Zea mays
MSTIAASRRSSSSSSSATAKRPAIADGPGGPKATAAQAKKRVALGNITNVAARGGRASVGGSLGNVMAPTSSAKLNPTVPLKKPSLATSARSVSSSIRGSADKPASIKPAPPVARHGSATQRHNNVPPPKVPTIADVPSRAPALVSCTGLVSPGRSGDSVSSDETMSTCDSMKSPDFEYVDNQDTSMLASLQRRTSEHLRISEDRDVEENKRKKNAVAPMEIDRICDVDSEYEDPQLCATLASDIYMHLREAETKKRPSTDFMETIQKDVNPSMRAILIDWLVEVAEEYRLVPDTLYLTVNYIDRYLSGNEISRQRLQLLGVACMLIAAKYEEICAPQVEEFCYITDNTYFRDEVLDMEASVLNYLKFEMTAPTAKCFLRRFARAAQACDEDPALHLEFLANYIAELSLLEYSLLSYPPSLIAASAIFLARFILQPTKYPWNSTLAHYTQYKPSKLSECVKALHRLCSVGSGSNLPAIREKYSQHKYKFVAKKQCPPQIPTEFFRDTTC

SEQ ID NO: 79 - protein, 298 aa, Zea mays
MASSKRRFGAAGGADKKDLFHVVHKVPAGDSPYVVAKHLQLVEKQPDVAIVWFWKAINSGDRVDSALKDMAMVMKQQDRSEEAIEAIRSFRHLCSKQAQESLDNLLIDLYKKCGKVEEQIELLKQKLKSIYLGEAFNGKATKKARSHGKKFQVSIQQETSRILGNLGWAYMQQNNFEAAELVYRKAQAIEPDANRACNLGLCLIKQGRHDEARQALEDVRLRRIYGSEDGKVVARAEQLLRELNPLQCVSSPFQVGLSVHEGIMGKPDLVVMNEWTPFRSRRLPVFEEIATFRDQMAC

SEQ ID NO: 80 - protein, 1096 aa, Zea mays
MAAADGDALIAAFAVSKGGIVLKHIFLNAPPPEAMCGSGGREVESDEEDPPVMVGRHPDCHVLVDHPSVSRFHLELRCRRRQSLITVTDLHSVHGTWVSGRRIPPNTPVDLATGDTLRLGASKREYKLLWLSLREAFEMDDLMYMPSLPEEDKEEPYVKEPSSKLLPGHRDSVNMETHQDTSEQIVSEDIAFPAKVAPSAPPLSEFLQPFFVEEHSLSQFHEKRNGVTEEKLVDKNQISESFGSLIIQEMPGTLTNAGKSIQSGEQEDASNKVSKRSKLKSVKSLRVDTGRSSERSSTLSHSFQKGDQNDIVVCSQSCGTECAVCIALFGISEIEKAEEKEELIAEDNVDMNPPASMIMEGNMNERKPDNYIPQDPIGAKLQKKLGLLDSALPLHFKDDVFADKEIPQWNVASVHTESELLSEYLIIPEVKHDDLNHLNLEEGLSKSENINPNKITEGPGNCQLEGTIRGNLFDNLDTDGIEEGEEICPLDKDEITPNGSGNIIMERSNIVLKPTISQQLMDSISPLNLDHGDFSENENSMLNTGDQMKLNEPVSENLNPLIPTDEKYLKSQTEECMPISYLEFKDDILLDRENSVLAPRKYEAMSPVRQENLFSDKENVTPASKVKTVVRGVLGTRMDNSVSAANASNKNKVLGSRVDNSVSTENSSNKKQCELSSKSKKVHTVDFDVFYSDKENLTPISSGGMKARKCFPKDLSVDLDQDQEAFCSDKENLTPLSSAARKTRDMSGNLTRVESAVTKKRVVGRLPFQTLVSNSPLRPASSHDCTCAVARPAGVAAGDLAIKLEDKLNDLSCNGHESGSAGEGMKTWTMVANTDSLLDDESRKAIMLLKGLKGTRLFIPRIVIRELDSMKQREGLFRRSTKATSILQWIEECMARESWWIHVQSSADMFPVAPTPPATPSAQRIDEEIEISSGSFNPMMALFGPRSSAALADMISPRPEDRVLDCALLVSRVRSNEKVVVLSNSVTLKIKAMAEGLPCEGAKEFRESLVDPSSRRFMWAASAPRGSAWSCLDASALAENYYNSRHHAMKRRVLVAARPSESEAAKGLKLILRHNSLYAQATDAVNKTPLVSLAAV

SEQ ID NO: 81 - protein, 488 aa, Solanum tuberosum
MMTTQNRRSSVSSAVAKRQAMAANSSGLENNQTGKLNAGAAKKRPALSNISNHTTVSARNSISHSSKLAPCTSKIVSIKKNISSGTAVLPASSSVRPSSKPVSIQRSDAVVPKITAIPLPATCSMDISPSHSDGSLVSMDESMSTSDTVRSPEVEYIDDLESAAVDSIEKKACSTLYISEHIKAAAADICKRDVLVDLESGDKIVNIDNNLVDPQLCATMACDIYTHLRASEAKKRPSTDFMAKVQKDINPSMRAILIDWLVEVAEEYRLVPDTLHLTINYIDRYLSGNLMDRQRLQLLGVACMMIASKYEEICAPQVEEFCYITDNTYFKEEVLQMESTVLNYLKFEMTAPTAKCFLRRFVRAAQGLNEVLSLQLEHLASYIAELSLLEYNMLSYAPSLIAASAIFLAKYILLPSVKPWNSTLRHYTLYQPSDLQDCVMALHSLCCNNNNSSLPAVREKYSQHKYKFVAKKYCPPTVPVEFFQNISS

SEQ ID NO: 82 - protein, 487 aa, Solanum tuberosum
MMTTQNRRSSVSSAVAKRQAMAANSSGLENNQTGKLNAGAAKKRPALSNISNHTTVSARNSISHSSKLAPCTSKIVSIKKNISSGTAVLPASSSVRPSSKPVSIQRSDAVVPKITAIPLPATCSMDISPSHSDGSLVSMDESMSTSDTVRSPEVEYIDDLESAAVDSIEKKACSTLYISEHIKAAADICKRDVLVDLESGDKIVNIDNNLVDPQLCATMACDIYTHLRASEAKKRPSTDFMAKVQKDINPSMRAILIDWLVEVAEEYRLVPDTLHLTINYIDRYLSGNLMDRQRLQLLGVACMMIASKYEEICAPQVEEFCYITDNTYFKEEVLQMESTVLNYLKFEMTAPTAKCFLRRFVRAAQGLNEVLSLQLEHLASYIAELSLLEYNMLSYAPSLIAASAIFLAKYILLPSVKPWNSTLRHYTLYQPSDLQDCVMALHSLCCNNNNSSLPAVREKYSQHKYKFVAKKYCPPTVPVEFFQNISS

SEQ ID NO: 83 - protein, 483 aa, Solanum tuberosum
MMTTQNRRSSVSSAVAKRQAMAANSSGLENNQTGKLNAGAAKKRPALSNISNHTTVSARNSISHSSKLAPCTSKIVSIKKNISSGTAVLPASSSVRPSSKPVSIQRSDAVVPKITAIPLPATCSMDISPSHSDGSLVSMDESMSTSDTVRSPEVEYIDDLESAAVDSIEKKACSTLYISEHIKAAAADICKRDVLVDLESGDKIVNIDNNLVDPQLCATMACDIYTHLRASEAKKRPSTDFMAKVQKDINPSMRAILIDWLVEVAEEYRLVPDTLHLTINYIDRYLSGNLMDRQRLQLLGVACMMIASKYEEICAPQVEEFCYITDNTYFKEEVLQMESTVLNYLKFEMTAPTAKCFLRRFVRAAQGLNELEHLASYIAELSLLEYNMLSYAPSLIAASAIFLAKYILLPSVKPWNSTLRHYTLYQPSDLQDCVMALHSLCCNNNNSSLPAVREKYSQHKYKFVAKKYCPPTVPVEFFQNISS

SEQ ID NO: 84 - protein, 614 aa, Solanum tuberosum
MWRNNERVYMSPARGFLTPPPKWRSPASDKDQRWPTHAQSAKADLFHVIHKVPSGDSPYVRAKHVQLIDKDPGKAISLFWAAINSGDRVDSALKDMAVVMKQLDRSDEAIEAIKSFRNLCPSESQESIDNILIELYKRSGRLEEEIELLELKLKNVEEGIAFGGKRTKIARSQGKKVQITIEKEYARLLGNLAWSYMQLNNFKLAEEYYRKALSLESDKNKQSNLAICLMHMNKIAEARFLLQSIKASDRWQMDESCTKSFERATQMLAELETHGIQNSKEQVEEMREVRIDSSTSDEHDCRGHEKTHPPPFTASGPPKHFLTQPRRYSCSLNDGGWLNKDSVSACSRRLLFEQTSNNENVQLVVNHNFNKLISANDMSEGASLVRGQVLTRSWGNGANVESECDLQPPYSKWKNNSSGNDGSDQISLELSKSPTESLADITSARKYSGDGSKDCWSSTLTYRDTVTLEDTTEHLESTNLKPLNLPACTSKKSWADMVEEDELGLQFHETPGKYSDENENIDSNIINLSQNIDTLRLNEGYHTQPGREARRSLCFDQNDRKEKCSSDFQGKVLKSGSLNSLPPIGDIAYQTPVTLMRRNRLQVFRDITPESPKP

SEQ ID NO: 85 - protein, 835 aa, Solanum tuberosum
MADKLEISSIEEDKKIPVFTVLKNGAILKNIFLLDNPPPGSNQESEIEEILVVGRHPDCNITLEHPSISRFHLRIHSKPSSLSLSVTDLSSVHGTWISGKKLESGVKVELKEGDRMQLGGSSRVYRLHWVPISHAYDLENPFVPTLGESEPEESTQEEQHQDESGFSLQNDQIQKEDYDMVQGLDSSFSGMSSLPRLRSLTPPAPPMLDKYGAANQNLPGNIHEEGEISLLQPAYQTDKENSTPEALLVSGQSPNENADGTPPRSQQRCSSIWSRRGKPSNVQIQTGKDRAMNEKIDMETEVESINREIAGTVSVSKDLFASGNKDKEEEVFTPDKENHTPSSLFLGSMKKSCLSEMTNRSDRKSVLSTMDETDEEIFTPDKENMTPETRRLRLMKKIGSQHQIKHPKLFKSSSLKLVVEPRFNQAAGCVSHKKEKLGSTTKSTQSDMDENDEEIFTLDKENMTPDTRLMRSMKKIGKLEDLKHLESFKFSLDNVVGTPFCSEKDNLTNKVLVEQKSTILASRYPARLEVNTVKNRMDRVPLQSLLVNYPVKTSSISPEENIKLRDYPIKHPETTELCPFSNESVMEQKRWTIVVDTGSLLNKESRKSLQLLQGLRGTYMIIPRTVIRELDCMKRRASLFRRTTEVSAALEWIEDCMINAKSWIHVQSCAEETRAVAPTPPATAPLSLFSEENDMFPVGLHQFSPHSGLMEFASPTAEDHILEYALFFKRTNRNGQLVLLSNDLTMKIKAMAEGLNCETAEEFRESLVNPFSERFLWKDSSPRGRTWSCEDDFVLRETYYHGPPKKPSVSGEAAKGLKLILLHNSHFRHHISTAS

SEQ ID NO: 86 - protein, 823 aa, Solanum tuberosum
MAEKQESLGKPNSQSEEEKKIPVFTVLKNGAILKNIFLLDNPPATHVAITANQEFEEILVVGRHPDCNIVLEHPSISRFHLRIHSNPSSHSLSVIDLSSVHGSWISGNKIEPGVRVELKEGDKMKLGGSRREYMLHWIPISRAYDLENPFVAPLGEAEPFKEMDEKEHQDENGFALQNEGDDLVQDQDSSFSCSSLLPYIKCPTPSAPPMPEEMKSSFPYGDEEVNINPLEKIHGESEISLLQPAYEPDQENSSPRALLVSGGFRTENADSTPVRSQQRCLNIWSRRGKCSSVQIQTGRDRAFIERVDIDTEVHTLDHEKVAMESVFNDPFSSGNKDEDHAFTPDKENHVPSSLLLGSEKNSCLTDGQTTKPMLSCCMDENGEENFTLDKVDFSANTHSPRSMKKMSCSEQIKNPKSLRSSPMKETFDSILTQAEGLEYYTENVGSSTIPPNMSKNSEQIFTPDKENMTPDSYSMRSIKKGNMEEVKQRKPFAKENLNDKVLEEKISASLSSRNKARMEVTMLKNRTDRVPFQPLLVTNSPNKTKSESPERKVKLNAKPVKCQEIMEAYPFANNNAREEKRRWTMVVDITSLLNKESRKALQLLQGLKGTCLIIPRIVLRELDYMKRRASFFRRATEVFAAIDWIDDCMVNAKSWVHVQSCVEETRPVAPTPPATAPPYFFNEENGIFPVGSVLSSPHCGLAEIVSPTADDHILEYALLFKRTNRDGQLVLLSNDLTMKIKAMAEGIICETAEDFQESLVNPFSERFLWKDSSPRGSTWSCVDDFVLGPLRKTSKSGQAAKGLKLILLHNSHYRQICSGSTVS

SEQ ID NO: 87 - protein, 485 aa, Solanum tuberosum
MACLFGCFKIKDVTLPATPGSSSESNRVSQSTPAKEPMVSRNRSPLSSLFTEENEETNTLGCKEVENLSLDTPMPQLDTNDLRNQAKFLKACGTLPETPAEIRNFSMKCKDLSAPMEEVEPLKVNSWPSDTTYQKLNLNSLSDQPTPVKIDGVKTHSGSFVHTPSSCLTDGQSDQSLSKSFINGNENSSTPIFIEVKANLARNDNKASAASPITAPTAQYRYRSVHFESESDLSSMSSKCISSETSQSSEQSESSGNYNASKYSPYPTPLKLSDEMQTPGTVFPAYLDRMGIAKTARIRSQYVYPVLNPVDDVSQLKELSDEDSFSTQDSNSRLFSSHTTDSDQRPEEATRISELGMSGFGESAANKDSKPPSINQVQSKQHLGPVHYGRTPRDRPIIGLVAAHWNDDETSRISPKWWDGNGIPNSTNKYKEDQKVSWHATPFEERLEKALSEETNISQRTQHSGTTPIAVHEAEESNTATSQVR

SEQ ID NO: 88 - protein, 609 aa, Glycine max
MFYSHQLLARKAPLGQIWMAATMHAKINRRKLDKLNIINICEEILNPSIPMALRLSGILMGGVVIVYERKVKLLYDDVTRFLVEINEAWKVKAAPDPTLLPKSKSKAKKEAVTLPGTGETNVADIEQSLQFSNTGTTNGFQHNAYFTMRLDSVDEPFISNGAREDQDQSELLHQVDAENITLFERFETFQANADPYNHLLFERFERFDIEGDEETQVNVTSADQIPTTLVPSPPHQDEPTRADTFQDHHPGHPDIQQPSEGMIPRQEPKRRGPNKRKRGQPSTIEMDYEQTIIPAHIYQHWLQNASDIVSRRGRKKKLTDVMSSTKIANLMKLPPVVLIGDLFTAENDNIYYPKAIMDLWIKSIQPPHDSPSERISAHHPPEPSFSSPPGVQNEDFMQFPSEDFDSRPDYQAPPMEKAREHILLDELTTSLLRGHEATPHISSGKAGDSVHSFPRPTSENGPASHSDFDSGRFKNKRYSSSANSSGGLEPLGGLEPLAEDVNFKLARLYEDGPTPDQELFVETGPTQTQANINHPSDKITDSIQAQMKAHFDTPGAPPVESLHNLAAGMTRTSAAQLFYQICVLASRDALKVDQKVPYGEIVFSRGLKM

SEQ ID NO: 89 - protein, 367 aa, Glycine max
MEGKRTRFQSESQPQSVILLTKIKEFTRALLADLTNGRSPLILIDRFRSYCTLSNSNCFCASDLPCGKELLTLRRKSHAHRLAVMLRVMLIVQKLLQENKHSSKRDIYYTYPSVFLDQSVVDQAINDICILMQCSRHNLNVVSAGNGLIMGWIRFSEGERIFDCISSPNTAHPVPVHVEEVQDIISLAQYILVVEKESVFQRLANDQFCNANHCIVITGRGYPDIPTRRFLRLLVENLCLPAYCLVDCDPYGFDILTTYRFGSMQMAYDTKHLRVPEIQWLGAFPSDSERYFVPNQCLLPLTAEDKRKVEAMLLRCYLEREVPQWRLELQLILQRGVKFEIEALSVHALSFLSESYIPSKIEGKLIM

SEQ ID NO: 90 - protein, 400 aa, Glycine max
MKINKACDLNSISVFPPPLVYPRKANNISSGLQASHSQHRSQPSQQSLSQGLSSQHGVLSHFSQNSLDEAVTTNDQRVGSQEHENSLRRISSLSRPTYSREESQAPNSRSSSNLMLKWNSADHKTGQLSEGLEHRIGIMETSLSRFTMILDSVQSDVMQVNKGTKEIILEVECIRQKLIAQDNSLQLMTKGQEEIKAIIERSLKSLAEQMSHVSKKEKLQDVYLAVSALPQLIEASLQNVQNDLRNITKEMQEISCNLKSVNQKDLVPTSLSSKSISKQITTPKMRQPLANEAKVSIQATVAPEVESGGWKPVKKERVTFSDRTSEKVQKKKEPHTQKYIRGGRDCAIVIESDEEIDGGFSCLVQENAGVNLLGNLTKEALEETERILRKARRRKRKSIF

SEQ ID NO: 91 - protein, 215 aa, Glycine max
MPESRDRRITVVDLAAAIARRRASFIYIDSPPLRTPQRTAAIGRGRASGSPGSQNTPPSTARRGRGRVPSRNVLPAWYPRTPLRDITVVVQAIERRRARSGEAEGQRIGSTDPASDRLVTEPSEPASADSAVKSPKSVGVKLRTPFGSKVPKIFLDISELPEDDESETLTPQKKLLNNIDQVEEAVREELKKLKRTPSAKKAEREKRVRTLMSMR

SEQ ID NO: 92 - protein, 218 aa, Glycine max
MPQSRHRRVTVVDLAASLARRRVSFIFNEAPTLRTPPRTAAFGRGRARASPRSQNIPPSTARRGRGRVPLRSVLPAWFPRTPLRDITAVVQAIERRSARLGEVEGQRIGNTDPASDRLVSEPSEPASASASASAVKSPKSVGVKLRTPFGSKVPKIFLDISELPEHDESEALTPQKKLLDNIDQVEEAVREELNKLKRTPSAKKTEREKRVRTLMSMR

SEQ ID NO: 93 - protein, 480 aa, Glycine max
MSTQNRRSSFSSSTTSSLAKRHVSASTTSSSLAAPANMAKKRPPLSNLTNTVAHRNSSSLVPCAAKFAKTKKEVVPACSGNKRPTLANVKPTSAAIFPKANSFPVRDEAPPPVATVTVPAPVVDVSPSKSDAMSVSMDESMSSCDSFKSPDIEYVDNIDVAAVDSIERKTFSNLNISDSTEGNICSRDILVELEKGENFVNVDNNYADPQLCATFACDIYKHLRASEAKKRPSTDFMERIQKEINPSMRAILIDWLVEVAEEYRLVPDTLYLTVNYIDRYLSGNVMNRQRLQLLGVASMMIASKYEEICAPQVEEFCYITDNTYFKEEVLQMESAVLNFLKFEMTAPTVKCFLRRFVRAAQGVDEVPSLQLECLTNYIAELSLMEYSMLGYAPSLIAASAIFLAKFILFPSKKPWTSTLQHYTLYKPSDLCVCVRDLHRLCCNSPNSNLPAIREKYSQHKYKYVAKKYCPPSIPPEFFQN

SEQ ID NO: 94 - protein, 601 aa, Glycine max
MLFERGSPARCYMTPPPQRTSPWKPPHSRSSSVPFSERKMSPNSVNKSDIFHIIHKVPAGDSPYVKAKQVQLVDKDPGRAVSLFWAAINAGDRVESALKDMALVMKQLNRSDEAIEAIRSFRHLCPSDSQDSLDNILVELYKRSGRVDEEIAMLCHKLKQIEDGLTFVGRTTKQARSQGKKIQITAEQEISRILGNLAWAYLQKGDYKTAEEHYRKALSFEVDRNKQCNLAICLIHMNKIKEAKFLLQAVRTATKNRKMDDSFAKSFERASQMLIEIETSSSQNAAFSMTTQCPPQSFENSIRMSSDSVQSRSENRSEISEGDAPHARRRLYQSPDPSRRDLSVPCTKPKRCSWGFNNGHRREAWGDANSDYKPTFGTPPNDKHVTRMLNSRENGFSSPANGNGKAATLEDPAILKHEATKVTSSDSLHTLNTESATEFTEKEKSAADDSSYGSILSESHVTVVNGVNEFASGKRKPEKKSWADIVEEEQNEENDFFSGFTNFDDKNGAEVFNDENEDSNIIYPSPWPQNQPDEWSSKKLESLDKKDGHYASESAILSRNPTARRSLCFNPELIGEKRLTRRSRLQVFQDITLLPETPRFA

SEQ ID NO: 95 - protein, 602 aa, Glycine max
MQFERGSMARCYMTPPPQPPSSWKPPHSQSPSVPFSERKKSPNSANKIDLFHIIHKVPAGDSPYVKAKQVQLVDKDPGRAISLFWAAINARDRVESALKDMALVMKQLNRSDEAIEAIRSFRHLCPSDSRDSLDNILVELYKRSGRIDEEIAMLHHKLKQIEDGLTFVGRTTKQARSQGKKIQITAEQEISRILGNLAWAYLQKGDYKAAEEHYRKALSFEVDRNKQCNLAICLIHMNKIKEAKFLLQAVRTATKNRKMDDSFAKSFERASQMLIEIETSSSSQNAAFSMTTMSQCSPQSFENSIRKSSDRVQSGSESRSETSEGDVPHARRRLYQSPDPSRRDLSVPCTKPKRCSWGFNTGYRREAWGDVNSDYKPSFGTPPNDKHATRMLNSRENGLSSPANGKWRAMTLEDQAILKHEATTATSSDSLHALNTEVAMEFTEKEKSAADDSSYRSILSESHVTVVNGANEFASGKRKPEKKSWADIVEEEQNEEYDFLSGFTNFDDKNGAEVFNDENEDSNIIYPSSWLQNLPEWSNKKLESSDKKDDYASESTILSRNPTARRSLCFNPELIGEKKLTRRSRLQVFQDITLLPETPRFA

SEQ ID NO: 96 - protein, 442 aa, Arabidopsis thaliana
MSSSSRNLSQENPIPRPNLAKTRTSLRDVGNRRAPLGDITNQKNGSRNPSPSSTLVNCSNKIGQSKKAPKPALSRNWNLGILDSGLPPKPNAKSNIIVPYEDTELLQSDDSLLCSSPALSLDASPTQSDPSISTHDSLTNHVVDYMVESTTDDGNDDDDDEIVNIDSDLMDPQLCASFACDIYEHLRVSEVNKRPALDYMERTQSSINASMRSILIDWLVEVAEEYRLSPETLYLAVNYVDRYLTGNAINKQNLQLLGVTCMMIAAKYEEVCVPQVEDFCYITDNTYLRNELLEMESSVLNYLKFELTTPTAKCFLRRFLRAAQGRKEVPSLLSECLACYLTELSLLDYAMLRYAPSLVAASAVFLAQYTLHPSRKPWNATLEHYTSYRAKHMEACVKNLLQLCNEKLSSDVVAIRKKYSQHKYKFAAKKLCPTSLPQELFL

SEQ ID NO: 97 - protein, 488 aa, Solanum tuberosum; identical to SEQ ID NO: 81

SEQ ID NO: 98 - protein, 501 aa, Citrullus lanatus
MSAHNRRPSFSSSTSSSLAKRQASSASSSDNVGKVMAVPPHLAKKRAPLGNLTNFKNVSHSAAKSSVPPAIMVPCATKAVKARKSSPARTRSTNLTTTNTTTLLDVKTTNAIAPSNVTAFSRTDATAVSSSMDVSPSKSDGVSVSLDETMSTCDSFKSPDVEYLDNTDVPAVDSVERKTKSSLCISGPAPISSQAPVKGSICNRDVITEMETDGDIVDVDTDFMDPQQCATIACDIYKHLRASEAKKRPSTDFMEKIQKDINSNMRAILIDWLVEVAEEYRLVPDTLYLTVNYIDRYLSGNSMDRQRLQLLGVACMMIASKYEEICAPQVEEFCYITDNTYFKDEVLEMESSVLNYLKFEMTAPTPKCFLRRFVRAAQGATDEVSSMQLECLANFLAELSLIEYSMLCYAPSLVAASAIFLAKFILLPTKRPWNSTLQHYTHYQPSDLVDCVKDLHRLCCNTHNSSLPAIREKYSQHKYKHVAKKYCPPTIPPEFFQNQTQ

SEQ ID NO: 99 - protein, 498 aa, Prunus avium
MSTQNRRSAFPSSLAKRQALSSSSSSSSASGNVGKVVAAEPHLAKKRAPLSNLTNRNSVSNNGLRSSGGSSCTLVPCTNKIAKVKKGPPACTGNKGVSASSLPASFNVKPSAVVHPKVTPAPRSDEAVPSSFALPGPCSMDVSPSKSDGNSVSMDETMSSCDSFKSPEVEYIDNNDVPAIDSINRKTFSNLYISDHAETTGIACKREVLVELEAEDKIADLDNSLMDPQLCATIACDIYKHLRASEVKKRPSTDFMEKTQKDINPSMRAILIDWLVEVAEEYRLVPDTLYLTVNYIDRYLSGNPMNRQRLQLLGVACMMIASKYEEICAPQVEEFCYITDNTYFKEEVLQMESDVLNYLKFEMTAPTTKCFLRRFVRAAQGANEGISMHLECLANYLAELSLLEYSMLCYAPSLVAASAIFLANFILLPSKRPWNATLQHYTLYQPSDLRDCVKDLHRLCSNSQSSSLPAIREKYSQHKYKHVAKKYCPPTIPSEYQT

SEQ ID NO: 100 - protein, 490 aa, Vaccinium corymbosum
MSNQNRRSSVTTSTSSSLAKRHAENLGKAQPYVAKKRPALANVTNQRHGSQNGPRSLVSGSSNMVPCMTKVANAKKISSTCISNASFSRSTLPVAVNVKPSVAVSSKSSPFIKYDDFPRIPAPDAPCGMDISSPHSDGSSVSMDETMSSCDSLKSPEFEYVDNTEIAAVDSIERRTSSKLYISEHMEAAETVCKRDIIVGVGTDEEFVDVDDNFMDPQLCATMACDIYKHLRATEAKKRPSTDFMERVQKDINASMRAILVDWLVEVAEEYRLVPDTLYLTINYLDRYLSGNTMDRQRLQLLGVACMMIAAKYEEICAPQVEEFCYITDNTYFKEEVLQMESAVLNYMKFEMTAPTAKCFLRRFVRAAQGVNEAPSMQLECLANYLAELSLLEYNMLCYAPSVIAASAIFLAKYILIPSKRPWNSTLRHYTHYQPCDLSDCVKALHSLCCNSQSSSLPAIREKYSLHKYKFVAKKYCPPAIPAEYFQNLS

SEQ ID NO: 101 - protein, 490 aa, Solanum lycopersicum
MMTTQNRRSSAVAKRQAMAANSSGLEHNQTGKLNAKKRPALSNISNNTTVSARNSVSHSSKLAPCTSKIVNIKKNTSACNSNAVSSGTAVLPASSSVRPSSKPVSIQRSDAVVPKITVIPVPATCSMDISPSHSDGSLVSMDESMSNSDTVRSPEVEYIDDHELAAVDSIEKKACSTLYISEHVKAAAADICKRDVLVDLESGDKIMNIDNNLVDPQLCATMACDIYKHLRASEAKKRPSTDFMAKVQKDINPSMRAILIDWLVEVAEEYRLVPDTLHLTINYIDRYLSGNLMDRQRLQLLGVACMMIASKYEEICAPQVEEFCYITDNTYFKEEVLQMESAVLNYLKFEMTAPTAKCFLRRFVRAAQGLNEVLSLQLEHLASYIAELSLLEYNMLCYAPSLIAASAIFLAKYILLPSVKPWNSTLRHYTLYQPSDLRDCVLALHSLCCNNNNSSLPAVREKYSQHKYKFVAKKYCPPTVPVEFFQNISS

SEQ ID NO: 102 - protein, 490 aa, Gossypium barbadense
MSTQKGNRRSSFSSSTTSSLAKRHASSSENVGKVMASLAKKRVPLTNITNQKQSSSSKSSALASSLVPSSNKIQKARKTQSVTNVGLSGHVLPPTNERRSSVLPPKVVPSLPTGNEVVLPPPPVQCSMEFSPSKSDGLSSVSLDETMSTCDSFKSPEVEYIDNHDVSAIGSIERKTFSNLCISDHDVVATGASICNRDAVAEKEADDMVIDVDDNFMDPQLCATFACDIYKHLRASEVKKRPSTDFMETIQKDINSSMRAILIDWLVEVAEEYRLVPDTLYLTVSYIDRYLSGNTMNRQRLQLLGVACMMIASKYEEICAPQVEEFCYITDNTYFKDEVLEMESSVLNYLKFEMTAPTAKCFLRRFVRAAQGINEVPSMQLECMANYIAELSLLEYSMLCYPPSLIAASAIFLAKFILLPSKKPWNSTLQHFTLYKPSDLFECVKELHRLCCNNNNSTLPAIREKYSQHKYKCVAKKYCPPSIPSDFFHN

SEQ ID NO: 103 - protein, 1086 aa, Rubus occidentalis
PKEDTAASHGGFGLLYREETVTISLSPQLRFGPKMSTRNRSVPSSSAKRAPIPQNRNKKAMAAKPQVAKKRTALSDVTNQRNGSQSGPRTSGLPSKPMVPCTTKIAKNNTGLSGKGFPSSLSAKSSILVPFSDTSVSGIVESIETVATNHSLSSCGTVLPAPNSIIHALRSKDVSPSRSASGSVSLDESMSTSDSLKSPEFEYIDNEDISEVKSIENKTTNSLYISDLPQIETTNGIVDIDNNFTDPQFCATIASDIYKYLRESEFLGFQTNRRPSTDFMERVQKDINAGMRAILIDWLVEVAEEFRLVPDTLFLTVNYVDRYLSGNVMKRQRLQLLGVACMMVAAKYEEIHVPEVEEFCYITDNTYFKEEVLQMESSVLKHLKFEMTAPTAICFLRRFCFVAERTSEVSISDLSAFSCLILCTDNQVPSLLFQCLAHYIAELSLLEYSMLGYAPSLIAASAAFLAKYILSPSQRPWNSSLRHYTRYHASDLCDCVKVLHHLCCNGSGSNLAAVREKYCQHKNRRSAFPSSLAKRQASSSSSNVGKFPVAEPRLAKKRAPLSNLTNRSNVPNNALKSSLPRIVSLSVYKRKGYPVSGILGLCTCIYFAFSAMKFFNESSYLTPPPKLYFEMSYHTPLNANSPPNVATIDTKQVPCASKIAKVKKGSPASTSNKGISGSNLSASFSVKSTAVVLPKVTSIQTSDEVFPKFTAVQRSDEGSVAIPVPCNMDDSPSKSDGDSVSMDETMSTCDSFKSPEVEYIDNNDGQAIDSIDRKTFSHLHISDHAETTGIVCSRELLVDLEADGSIADIDNRFMDPQLCATFACDIYKHLRASEVKKRPSTDYMDKIQKDINPSMRAILVDWLVEVAEEYRLVPDTLYLTVNYIDRYLSGNPMNRQRLQLLGVACMMIASKYEEICAPQVEEFCYITDNTYFKEEVLEMESSVLNYLKFEMTAATTKCFLRRFVRAAQGANELPSMQLECLANYLAELSLLDYSMLSYAPSLIAASAIFLANYVTLPSKRPWNATLQHYTLYQPSDLRDCVKDLHRLCCDNNSSSLPAIREKYSQHKYKYVAKKYCPPTIPIEYQA

SEQ ID NO: 104 - protein, 515 aa, synthetic construct
  REGION 1..515: note = Synthetic Construct
  VARIANT 54: note = Xaa = N or D
  VARIANT 180: note = Xaa = N or D
  VARIANT 232: note = Xaa = N or D
  VARIANT 318: note = Xaa = N or D
  VARIANT 362: note = Xaa = Q or E
  VARIANT 410: note = Xaa = L or I
  VARIANT 416: note = Xaa = L or I
  VARIANT 1..515: note = Xaa = Any Amino Acid, or no amino acid (except at positions 54, 180, 232, 318, 362, 410 and 416)
MXXXXGNRXXXXXXXXXXXXXXXXXXSXXXAXXXXGKXXAXXXXXAXXRXXLXXXXNXXXXSXXXXXXXXXXXXXXXXXXXKXXXXXKXXXXXXSXXXXXXXXXXXXXXXXXXXXXXXXXXXXXXXXXXXXXXXXXXXXXXXXXXXXSXXXSDGLXXVSXDXXXSXXDXXXXXXXXYXDXXXXXAXXSXXXXXXXXLXISXXXXXXXXXXVKGSICXRXXXXXXXXXXXXXXXDXXXXDPQXCAXXACDIYXHLRXXEXXKRPXXDXMXXXQXXINXXMRXILXDWLVEVAEEYRLXPXTLXLXXXYXDRYLXGNXXXXQXLQLLGVXCMMIAXKYEEXCXPQVEXFCYITDNTYXXXEXLXMESXVLNYXKFEXTXPTXKCFLRRFXRAAQGXXXXXXSXXXEXXAXXXXELSLXXYXMLXYXPSXXAASAXFLAXXXLXPXXXPWNXTLXHXTXYXXXXXXXCVXXLXXLCXXXXXSXXXAXRXKYSXHKYKXXAKKXCPXXXPXXXXXNXXX

SEQ ID NO: 105 - protein, 509 aa, Zea mays; identical to SEQ ID NO: 78

SEQ ID NO: 106 - protein, 502 aa, Zea mays
MSTHAASRRSSSSSAVAKRPAIAEGATKAAGPGPTAAQAKKRTALVNITNVAAPGARVAAVGKVAPPVTGAKLNPATSGAPLKKPSLANARTIRGSAAKSASIKPAPPVSRHDSSSEQKHNVLVPTTVHVPSRAPALVPYSSFVSPGRSRDSVSTDETMSTCDSMKSPDFEYIDNGGCSMLASLQRRADEHLRTSEDRDVEENKWKKNGPAPMEIDSICEVDSNLEDPQLCAALASDIYMHLREAEMKKRPSTDFMKTIQKDVNPSMRAILIDWLVEVAEEYRLAPDTLYLTVNYIDRYLSGNEINRQRLQLLGVACMLIAAKYEEICAPQVEEFCYITDNTYFRDEVLEMEASVLNYLKFEMTAPTAKCFLRRFARSAQACDEDPALHLEFLANYIAELSLLEYSLLSYPPSLIAASAIFLARFVLQPTKYPWNSTLAHYTQYKPSELSECVKTLHRLSSVGPGSNLPAIREKYSQHKYKFVAKKQCPPQIPAEFFRDAAC

SEQ ID NO: 107 - protein, 505 aa, Sorghum bicolor
MSTIAASRRSSSSSSAAAKRPAISEGAGGPKVAAAQAKKRVALGNITNVAARGGRAVVGGGLGNAAPPTTSAKLNPAVPLKKPSLASARSVSSIRGQAVKSASIKPAPPVSRHGSAIQKHNVPPPKVPTIADVPSHAPALVPCTGLVSPGRSGDFVSIDDTMSTCDSMKSPDFEYIDNQDSSMLASLQRRTNEHLRITEDRDVEENKWKKNAIAPMEIDRICDVDNEYEDPQLCATLASDIYMHLREAETKKRPSTDFMETIQKDINPSMRAILIDWLVEVSEEYRLVPDTLYLTVNYIDRYLSGNEINRQRLQLLGVACMLIAAKYEEICAPQVEEFCYITDNTYFRDEVLDMETSVLKYLKFEMTAPTAKCFLRRFARAAQACDEDPALHLEFLANYIAELSLLEYNLLSYPPSLIAASAIFLARFILQPTKYPWNSTLAHYTQYKPSELSDCVKALHRLCSVGSGTNLPAIREKYSQHKYKFVAKKQCPPQIPTEFFRDATC

SEQ ID NO: 108 - protein, 502 aa, Sorghum bicolor
MSTRAASRRSSSSSAAAKRPAMAEGAKAAGTAAAQAKKRAALGNITNVVAAPAGRAAALGKVAPPVTGAKLNSATSAVPLKKPSLANARNASSIRGSAVKSASIKPAPPVPRLDSSTRPKHNVPVPATVHVPSRAPALVPCSSFVSPARSGDSVSIDETMSTCDSMKSPDFEYIDNGDSSMLASLQRRADEHLRISEDTDVEENKWKKNAPAPMEIDRVCDVDNDLEDPQLCATLASDIYMHLREAETKKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDEVLEMEASVLNYLKFEMTAPTAKCFLRRFARSAQACDEDPALHLEFLASYIAELSLLEYNLLSYPPSLIAASAIFLARFILQPTKYPWNSTLSHYTQYKPSELSDCVKALHRLFSVGPGSNLPAIREKYSQHKYKFVAKKQCPPQIPTEFFRDTT

SEQ ID NO: 109 - protein, 505 aa, Sorghum bicolor
MSTFAASRRSSSSAASKRPAMDENAAGGPRWTRPQATKRVALGNITNVAAPAPGRRAAVGKVAPPATTRAKLNPATSAAPLKKPYPANARNVSSIRGSAVKSASIKPGPPVSCLCSSTMQQKHNVPPPKVPTTVNVPSRAPALVPCSSFMSPGRSGDSVSMDETMSICDSMKSPDFEFIDNGDSSVLASLQRQANEHLRISEENKWKKNAPAPFEIDHICDVDNDYEDPQLCATLASDIYMHLREAEMKKSKRPSTDFMETIHKSVNPSMRAILIDWLVEVAEEYRLVPDTLYLTVNYIDRYLSGNEIDRQRLQLLGVTCMLIAAKYEEICAPQVEEFCYITDSTYFRDDVLEMEASVLNYLKFEMAAPTPKCFLRRFARAAQACDEDPALHLEFLANYIAELSLLEYNLLSYPPSLIAASAVFLARYVLQPTKYPWNSTLAHYTQYKPSELSDCVKALHRLFSVGPGSNLPAIREKYSQHKYKFVARKQCPPSIPTEFFRDVTW

SEQ ID NO: 110 - protein, 478 aa, Oryza sativa
MAAKRPAAGEGGGKAAAGAAAAKKRVALVNITNVAAAANNAKFNSATWAAPVKKGSLASGRNVCTNRVSAVKSASAKPAPAISRHESAPQKESVIPPKVLSIVPTAAPAPVTVPCSSFVSPMHSGDSVSVDETMSMCDSMKSPDFEYIDNGDSSSVLGSLQRRANENLRISEDRDVEETKWNKDAPSPMEIDQICDVDNNYEDPQLCATLASDIYMHLREAETRKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDEVLEMEASVLNYLKFEVTAPTAKCFLRRFVRVAQVSDEDPALHLEFLANYVAELSLLEYNLLSYPPSLVAASAIFLAKFILQPTKHPWNSTLAHYTQYKSSELSDCVKALHRLFSVGPGSNLPAIREKYTQHKYKFVAKKHCPPSVPSEFFRDATC

SEQ ID NO: 111 - protein, 508 aa, Oryza sativa
MSSNLAASRRSSSSSSVAAAAAAKRPAVGEGGGGGGGKAAAGAAAAKKRVALSNISNVAAGGGAPGKAGNAKLNLAASAAPVKKGSLASGRNVGTNRASAVKSASAKPAPAISRHESATQKESVLPPKVPSIVPTAALAPVTVPCSSFVSPMHSGDSVSVDETMSTCDSMKSPEFEYIDNGDSSSVLGSLQRRANENLRISEDRDVEETKWKKDAPSPMEIDQICDVDNNYEDPQLCATLASDIYMHLREAETRKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDEVLEMEASVLNYLKFEVTAPTAKCFLRRFVRVAQVSDEDPALHLEFLANYVAELSLLEYNLLSYPPSLVAASAIFLAKFILQPTKHPWNSTLAHYTQYKSSELSDCVKALHRLFSVGPGSNLPAIREKYTQHKYKFVAKKPCPPSIPTEFFRDATC

SEQ ID NO: 112 - protein, 491 aa, Oryza sativa
MSSSLASRRSSSSSAAKRPAAGEGGGKAAAGAAAAKKRVALGNITNVAAAANNAKFNSATWAAPVKKGSLASGRNVGTNRVSAVKSASTKPASAISRHESAPQKESVLPPKVLRIVPTAAPAPVTVPCSSFVSPMHSGDSVSVDETMSTCDSMKSPDFEYIDNGDSSSVLGSLQRRANENLRISEDRDVEETKWKKDAPSPMEIDQICDVDNNYEDPQLCATLASDIYMHLREAETRKHPSTDFMETLQKDVNPSMRAILIDWLVEVAEEYRLVPDTLYLTVNYIDRYLSGNEINRQRLQLLGVACMLIAAKYKEICAPQVEEFCYITDNTYFRDEVLEMEASVLNYLKFEMTAPTAKCFLRRFVRVAQVSDEDPALHLEFLANYVAELSLLEYNLLSYPPSLVAASAIFLAKFILQPAKHPWNSTLAHYTQYKSSELSDCVKALHRLFCVGPGSNLPAIREKYTQHKYKFVAKKPCPPSIPTEFFRDSTC

SEQ ID NO: 113 - protein, 510 aa, Brachypodium distachyon
MSSTAAASRRLSSSAATATAKRPAMAENAGGAKPAGRMAAQQPKKRVALGNLTNVAAGAVGRTGCGKVAVVTAGNARLHSATSTVPVKKGSLASARNVITNRGSAVKSVSTRPTPVTSCRGSTTQKEIVPPPKLPAAMPIVAPPIVPCSSFVSPRHSADSMSTDETMSTCDSMKSPDFEYIDNGDSSVLDSLQRRANANLRISEDSDVEGAKWKKDATAPMEIDTICDVDNNYEDTQLCATLASDIYMHLREAETRKRPATDFLEKMQKDVNPSMRAILIDWLVEVAEEYRLVPDTLYLTVNYIDRYLSGNEINRQRLQLLGVACMLIAAKYEEICAPQVEEFCYITDNTYFKDEVLDMEASVLNYLKFEMTAPTPKCFLRRFVRVAQVCDEDPALHLEFLANYVAELSLLEYSLLAYPPSLVAASAVFLSKFILQPTKCPWNSTLAHYTQYKASELCDCVKALHRLFSVGPGSNLPAIREKYSQHKYKFVAKKQCPPLVPADFFRDATC

SEQ ID NO: 114 - protein, 501 aa, Brachypodium distachyon
MSSNAAASRRLSSATTATAKRPAMAESAGGAKLVDPVAVQQAKKRVALGNITNIAAGAMGRTGCGKVAVATPGNARLHSTTSAAPVKKGSLASVQNAIANRSLAVKSASTRPTPKAEDIVPPPKVPTVMPIAASAIVPCCSFASPQHSEDSISTDETMSTSDSMKSPDLEYIDNGDSLVLDSLQQRANANLRISEESDVEGTKWKKDATTPMEIDNISDVDDNYKDPQLCATLPSDIYMHLRDTETRKRPASDFLETMQKDINPSMRAILIDWLVEVSEEYRLVPDTLYLTVNYIDRYLSGNEINRQRLQLLGVACMLIAAKHEEICAPQVEEFCYITDNTYFKDEVLEMEASVINYLKFEMTAPTAKCFLRRFVRAAQVCDEDPALHLESLACYVTELSLLEYSLLVYPPSLVAASALFLSKFILQPTKSPWNSTLAHYTQYKASELCDCVKELQRLFCVAPGSKLPAIREKYSQHKYKFAAKKQCPPMVPADYFCDAAC

SEQ ID NO: 115 - protein, 515 aa, Hordeum vulgare
MSSNPAAPRRFSSAMSTATAKRPAVPEGAAGARPAAGPAAAAPQQQAKKRVALGNLTNVAAAAGGRAGCGKIAVVTAGNARLSSATAAAPVKKGSLPGARNANANRGSAVKSASTKPAPVTSRHEGSVQKESAPPPRKVPTVVPIAVPAVIPFSSFVSPGHSGDSISTDETMSSCDSMKSPDFEYIDNGDSSLLDSLQRRANENLRISDDRTVEGTKWKKDATAPMEIDNVCDVDDNYEDPQLCATLASDIYMHLREAETRKRPSTDFLETIQKDVNPSMRAILIDWLVEVAEEYRLVPDTLYLTVNYIDRYLSGNEINRQRLQLLGVACMLIAAKYEEICAPQVEEFCYITDNTYFKDEVLDMEASVLNYLKFEMTAPTAKCFLRRFVRAAQVCDEDPPLHLEFLANYVAELSLLEYSLLAYPPSLVAASAIFLSKFILQPAKHPWNSTLAHYTQYKPSELCDCVKALHRLFSVGPGSNLPAIREKYSQHKYKFVGKKQCPTSVPAEFFRDAAC

SEQ ID NO: 116 - protein, 510 aa, Triticum aestivum
MSSNSAAPRRFSSAMSTSTAKRPAVPEGARAAAGPAAAQQQAKKRVALGNLTTNVAAAAAGRAGCGKIAVVTTGNARLNSATSAAPVKKGALPSARNASANRGSAVKSAFTKPAPVTSRHESSVQKESVPPRKVPTVVPIAVPAVIPFSSFVSPGHSGDSISTDETMSSCDSMKSPDFEYIDNGDSSLLDSLQRRANENLRISDDRTVEGAKWKKDAAAPMEIDNVCDVDDNYEDPQLCATLASDIYMHLREAETRKRPSTDFLETIQKDVNPSMRAILIDWLVEVAEEYRLVPDTLYLTVNYIDRYLSGNEINRQRLQLLGVACMLIAAKYEEICAPQVEEFCYITDNTYFKDEVLDMEASVLNYLKFEMTAPTAKCFLRRFVRAAQVCDEDPPLHLEFLANYVAELSLLEYSLLAYPPSLVAASAIFLSKFILQPAKHPWNSTLAHYTQYKPSELCDCVKALHRLFSVGPGSNLPAIREKYSQHKYKFVGKKQCPTSVPAEFFRDAAC

SEQ ID NO: 117 - protein, 510 aa, Aegilops tauschii
MSSNSAAPRRFSSAMSTSTAKRPAVPEGARAAAGPAAAQQQAKKRVALGNLTTNVAAAAAGRAGCGKIAVVTTGNARLNSATSAAPVKKGALPSARHASANRGSAVKSAFTKPAPVTSRHESSVQKESVPPRKVPTVVPIAVPAVIPFSSFVSPGHSGDSISTDETMSSCDSMKSPDFEYIDNGDSSLLDSLQRRANENLRISDDRTVEGAKWKKDAAAPMEIDNVCDVDDNYEDPQLCATLASDIYMHLREAETRKRPSTDFLETIQKDVNPSMRAILIDWLVEVAEEYRLVPDTLYLTVNYIDRYLSGNEINRQRLQLLGVACMLIAAKYEEICAPQVEEFCYITDNTYFKDEVLDMEASVLNYLKFEMTAPTAKCFLRRFVRAAQVCDEDPPLHLEFLANYVAELSLLEYSLLAYPPSLVAASAIFLSKFILQPAKHPWNSTLAHYTQYKPSELCDCVKALHRLFSVGPGSNLPAIREKYSQHKYKFVGKKQCPTSVPAEFFRDAAC

SEQ ID NO: 118 - protein, 492 aa, Musa acuminata
MNRRPSISSSASAASASAKRPAATESASKMPAALAGQAKKRVALANISNQSKVARNPARPLGAKGANINTTSSASTKKGSLSSYRDTRSGGSSAVTSAAIKPDTNKSSNKSSLSINNGPKITVPACLVPCSTLKSPGHSRDSVSLDETMSTCDSMRSPDFEYIDKGDCSVITSLERQANKNLHISEHAAEAGSKLSIDVPMMMEVDDIIDVDTNHDDPQFCTTLAGDIYRHLRIAETKKRPSTDFMEKIQKDINASMRSILIDWLVEVAEEYRLVPDTLYLTVNYIDRYLSGNEINRQRLQLLGVSCMLIAAKYEEICAPQVEEFCYITDNTYFKDEVFQMEADVLKYLKYEMTAPTVKCFLRRFIRAAQGSDEVPALQLEFLASYVAEISLLEYSLLCYAPSLIAASAIFLARFILQPAKRPWNATLDHYTLYKPSDLSDCVKALHRLFCTSSGNNLPAIREKYSQHKYKFVAKKYCPASIPAEYFQDARN

SEQ ID NO: 119 - protein, 531 aa, synthetic construct
  REGION 1..531: note = Synthetic Construct
  VARIANT 1..531: note = Xaa = Any Amino Acid, or no amino acid
MSSXAAXXRSSSSSXSTAASAXAKRPAXXEGAGGAXXXAAXXAAAXQQQAKKRVALGNITTNVAAAAXGRAAVGGGCGKVAPXTTGNAKLNSATSAAPVKKGSLATSARNVXXNIRGSAVKSASXKPAPXXSRHXSSXXQKEXVXPPPKVPTXVPXAXXAPAXVPCSSFVSPGHSGDSVSXDETMSTCDSMKSPDFEYIDNGDSSSVLXSLQRRANENLRISEDRDVEEXKWKKDAPAPMEIDXICDVDNNYEDPQLCATLASDIYMHLREAETRKSKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDEVLEMEASVLNYLKFEMTAPTAKCFLRRFVRAAQVCDEDPALHLEFLANYVAELSLLEYSLLSYPPSLVAASAIFLAKFILQPTKXPWNSTLAHYTQYKPSELSDCVKALHRLFSVGPGSNLPAIREKYSQHKYKFVAKKQCPPSIPAEFFRDATC

SEQ ID NO: 120 - RNA, 20 nt, Solanum tuberosum
  source 1..20: mol_type = unassigned RNA
ctttcagtggaaagcttacg

SEQ ID NO: 121 - RNA, 20 nt, Solanum tuberosum
  source 1..20: mol_type = unassigned RNA
ctctttcttgagaacccaat

SEQ ID NO: 122 - RNA, 20 nt, Solanum tuberosum
  source 1..20: mol_type = unassigned RNA
gcactctataatgcagaaca

SEQ ID NO: 123 - RNA, 20 nt, Solanum tuberosum
  source 1..20: mol_type = unassigned RNA
aaggtcccctccaggctagc

SEQ ID NO: 124 - RNA, 20 nt, Solanum tuberosum
  source 1..20: mol_type = unassigned RNA
ccacatcacgtaaattaaaa

SEQ ID NO: 125 - RNA, 20 nt, Solanum tuberosum
  source 1..20: mol_type = unassigned RNA
atggaagcgctatctacctt

SEQ ID NO: 126 - RNA, 20 nt, Solanum tuberosum
  source 1..20: mol_type = unassigned RNA
aatatgcaacatgcctgaat

SEQ ID NO: 127 - RNA, 20 nt, Solanum tuberosum
  source 1..20: mol_type = unassigned RNA
aacgctttccctccattttc

SEQ ID NO: 128 - RNA, 20 nt, Solanum tuberosum
  source 1..20: mol_type = unassigned RNA
gcaggatttactcgttccat

SEQ ID NO: 129 - RNA, 20 nt, Solanum tuberosum
  source 1..20: mol_type = unassigned RNA
gttgtggaaaaggaatcagg

SEQ ID NO: 130 - RNA, 20 nt, Solanum tuberosum
  source 1..20: mol_type = unassigned RNA
tgtagctcttgcagctctgg

SEQ ID NO: 131 - RNA, 20 nt, Solanum tuberosum
  source 1..20: mol_type = unassigned RNA
agttgtggaaaaggaatcag

SEQ ID NO: 132 - RNA, 20 nt, Solanum tuberosum
  source 1..20: mol_type = unassigned RNA
agcaagaccttggctaaaga

SEQ ID NO: 133 - RNA, 20 nt, Solanum tuberosum
  source 1..20: mol_type = unassigned RNA
gttgagagttcttgtgatag

SEQ ID NO: 134 - RNA, 20 nt, Solanum tuberosum
  source 1..20: mol_type = unassigned RNA
ctctccctagactcaccaag

SEQ ID NO: 135 - RNA, 20 nt, Solanum tuberosum
  source 1..20: mol_type = unassigned RNA
ccacaactaaaacgtattga

SEQ ID NO: 136 - RNA, 20 nt, Solanum tuberosum
  source 1..20: mol_type = unassigned RNA
gagtgtagagtggccgccat

SEQ ID NO: 137 - RNA, 20 nt, Solanum tuberosum
  source 1..20: mol_type = unassigned RNA
aagcatgaattactctgcag

SEQ ID NO: 138 - RNA, 20 nt, Solanum tuberosum
  source 1..20: mol_type = unassigned RNA
cagttgatccagataatatc

SEQ ID NO: 139 - RNA, 20 nt, Solanum tuberosum
  source 1..20: mol_type = unassigned RNA
ggtttagatatctaatcgtc

SEQ ID NO: 140 - RNA, 20 nt, Solanum tuberosum
  source 1..20: mol_type = unassigned RNA
atagtatctgcaaatgaagt

SEQ ID NO: 141 - RNA, 20 nt, Solanum tuberosum
  source 1..20: mol_type = unassigned RNA
aagttatttcgtggattgag

SEQ ID NO: 142 - RNA, 20 nt, Solanum tuberosum
  source 1..20: mol_type = unassigned RNA
taggtcatcaatgattgtag

SEQ ID NO: 143 - RNA, 20 nt, Solanum tuberosum
  source 1..20: mol_type = unassigned RNA
aaaggtaaatctggatcgac

SEQ ID NO: 144 - RNA, 20 nt, Solanum tuberosum
  source 1..20: mol_type = unassigned RNA
cataatgtaccatcgtgaaa

SEQ ID NO: 145 - RNA, 20 nt, Solanum tuberosum
  source 1..20: mol_type = unassigned RNA
cgaagcatacccatgagaat

SEQ ID NO: 146 - RNA, 20 nt, Solanum tuberosum
  source 1..20: mol_type = unassigned RNA
accttgcatgaggaggaaga

SEQ ID NO: 147 - RNA, 20 nt, Solanum tuberosum
  source 1..20: mol_type = unassigned RNA
tgatttggtattttcaggtg

SEQ ID NO: 148 - RNA, 20 nt, Solanum tuberosum
  source 1..20: mol_type = unassigned RNA
gtggatcgactggagcggtt

SEQ ID NO: 149 - RNA, 20 nt, Solanum tuberosum
  source 1..20: mol_type = unassigned RNA
tatctctctctttgctgtga

SEQ ID NO: 150 - RNA, 21 nt, Solanum tuberosum
  source 1..21: mol_type = unassigned RNA
cctcatatcaactacacacga

SEQ ID NO: 151 - RNA, 21 nt, Solanum tuberosum
  source 1..21: mol_type = unassigned RNA
tctcaagcgagctcttgttct

SEQ ID NO: 152 - RNA, 20 nt, Solanum tuberosum
  source 1..20: mol_type = unassigned RNA
ctattgagtttcgagtgtag

SEQ ID NO: 153 - RNA, 20 nt, Solanum tuberosum
  source 1..20: mol_type = unassigned RNA
gacgtggggtgatcaaagaa

SEQ ID NO: 154 - RNA, 20 nt, Solanum tuberosum
  source 1..20: mol_type = unassigned RNA
agtctcttctcgtgtgtagt

SEQ ID NO: 155 - RNA, 20 nt, Solanum tuberosum
  source 1..20: mol_type = unassigned RNA
atcaccccacgtccaaaaac

SEQ ID NO: 156 - RNA, 20 nt, Solanum tuberosum
  source 1..20: mol_type = unassigned RNA
gctttatatagttctggatg

SEQ ID NO: 157 - RNA, 20 nt, Solanum tuberosum
  source 1..20: mol_type = unassigned RNA
ctccataggccattgaaaga

SEQ ID NO: 158 - RNA, 20 nt, Solanum tuberosum
  source 1..20: mol_type = unassigned RNA
ctttcaggaactgaccattt

SEQ ID NO: 159 - RNA, 20 nt, Solanum tuberosum
  source 1..20: mol_type = unassigned RNA
gcactttaccaacagatctg

SEQ ID NO: 160 - RNA, 20 nt, Solanum tuberosum
  source 1..20: mol_type = unassigned RNA
atgtcaatgcgctgattttc

SEQ ID NO: 161 - RNA, 21 nt, Solanum tuberosum
  source 1..21: mol_type = unassigned RNA
cttatgagtgttagtggctgc

SEQ ID NO: 162 - RNA, 21 nt, Solanum tuberosum
  source 1..21: mol_type = unassigned RNA
ctcccagattccacataccgc

SEQ ID NO: 163 - RNA, 20 nt, Solanum tuberosum
  source 1..20: mol_type = unassigned RNA
tgaaccgatcgatgtagatc

SEQ ID NO: 164 - RNA, 20 nt, Solanum tuberosum
  source 1..20: mol_type = unassigned RNA
ttggccgctccagcattcaa

SEQ ID NO: 165 - RNA, 20 nt, Solanum tuberosum
  source 1..20: mol_type = unassigned RNA
gaaggcaccatgtacgtcca

SEQ ID NO: 166 - RNA, 20 nt, Solanum tuberosum
  source 1..20: mol_type = unassigned RNA
atatgttctgagatgtagag

SEQ ID NO: 167 - RNA, 20 nt, Solanum tuberosum
  source 1..20: mol_type = unassigned RNA
ttccagcagcagatatatgc

SEQ ID NO: 168 - RNA, 20 nt, Solanum tuberosum
  source 1..20: mol_type = unassigned RNA
ttatgctccatcactcattg

SEQ ID NO: 169 - RNA, 20 nt, Solanum tuberosum
  source 1..20: mol_type = unassigned RNA
acaattaactacattgatcg

SEQ ID NO: 170 - RNA, 20 nt, Solanum tuberosum
  source 1..20: mol_type = unassigned RNA
gggcaacttgatggacagac

SEQ ID NO: 171 - RNA, 20 nt, Solanum tuberosum
  source 1..20: mol_type = unassigned RNA
ttacgatgcgatcatcatgc

SEQ ID NO: 172 - RNA, 20 nt, Solanum tuberosum
  source 1..20: mol_type = unassigned RNA
ctcagggactgctgttctgc

SEQ ID NO: 173 - RNA, 20 nt, Solanum tuberosum
  source 1..20: mol_type = unassigned RNA
tctattttttattccagcag

SEQ ID NO: 174 - RNA, 20 nt, Solanum tuberosum
  source 1..20: mol_type = unassigned RNA
gaaactcctaacagaactct

SEQ ID NO: 175 - DNA, 20 bp, Solanum tuberosum
  source 1..20: mol_type = unassigned DNA
ctttcagtggaaagcttacg

SEQ ID NO: 176 - DNA, 20 bp, Solanum tuberosum
  source 1..20: mol_type = unassigned DNA
ctctttcttgagaacccaat

SEQ ID NO: 177 - DNA, 20 bp, Solanum tuberosum
  source 1..20: mol_type = unassigned DNA
gcactctataatgcagaaca

SEQ ID NO: 178 - DNA, 20 bp, Solanum tuberosum
  source 1..20: mol_type = unassigned DNA
aaggtcccctccaggctagc

SEQ ID NO: 179 - DNA, 20 bp, Solanum tuberosum
  source 1..20: mol_type = unassigned DNA
ccacatcacgtaaattaaaa

SEQ ID NO: 180 - DNA, 20 bp, Solanum tuberosum
  source 1..20: mol_type = unassigned DNA
atggaagcgctatctacctt

SEQ ID NO: 181 - DNA, 20 bp, Solanum tuberosum
  source 1..20: mol_type = unassigned DNA
aatatgcaacatgcctgaat

SEQ ID NO: 182 - DNA, 20 bp, Solanum tuberosum
  source 1..20: mol_type = unassigned DNA
aacgctttccctccattttc

SEQ ID NO: 183 - DNA, 20 bp, Solanum tuberosum
  source 1..20: mol_type = unassigned DNA
gcaggatttactcgttccat

SEQ ID NO: 184 - DNA, 20 bp, Solanum tuberosum
  source 1..20: mol_type = unassigned DNA
gttgtggaaaaggaatcagg

SEQ ID NO: 185 - DNA, 20 bp, Solanum tuberosum
  source 1..20: mol_type = unassigned DNA
tgtagctcttgcagctctgg

SEQ ID NO: 186 - DNA, 20 bp, Solanum tuberosum
  source 1..20: mol_type = unassigned DNA
agttgtggaaaaggaatcag

SEQ ID NO: 187 - DNA, 20 bp, Solanum tuberosum
  source 1..20: mol_type = unassigned DNA
agcaagaccttggctaaaga

SEQ ID NO: 188 - DNA, 20 bp, Solanum tuberosum
  source 1..20: mol_type = unassigned DNA
gttgagagttcttgtgatag

SEQ ID NO: 189 - DNA, 20 bp, Solanum tuberosum
  source 1..20: mol_type = unassigned DNA
ctctccctagactcaccaag

SEQ ID NO: 190 - DNA, 20 bp, Solanum tuberosum
  source 1..20: mol_type = unassigned DNA
ccacaactaaaacgtattga

SEQ ID NO: 191 - DNA, 20 bp, Solanum tuberosum
  source 1..20: mol_type = unassigned DNA
gagtgtagagtggccgccat

SEQ ID NO: 192 - DNA, 20 bp, Solanum tuberosum
  source 1..20: mol_type = unassigned DNA
aagcatgaattactctgcag

SEQ ID NO: 193 - DNA, 20 bp, Solanum tuberosum
  source 1..20: mol_type = unassigned DNA
cagttgatccagataatatc

SEQ ID NO: 194 - DNA, 20 bp, Solanum tuberosum
  source 1..20: mol_type = unassigned DNA
ggtttagatatctaatcgtc

SEQ ID NO: 195 - DNA, 20 bp, Solanum tuberosum
  source 1..20: mol_type = unassigned DNA
atagtatctgcaaatgaagt

SEQ ID NO: 196 - DNA, 20 bp, Solanum tuberosum
  source 1..20: mol_type = unassigned DNA
aagttatttcgtggattgag

SEQ ID NO: 197 - DNA, 20 bp, Solanum tuberosum
  source 1..20: mol_type = unassigned DNA
taggtcatcaatgattgtag

SEQ ID NO: 198 - DNA, 20 bp, Solanum tuberosum
  source 1..20: mol_type = unassigned DNA
aaaggtaaatctggatcgac

SEQ ID NO: 199 - DNA, 20 bp, Solanum tuberosum
  source 1..20: mol_type = unassigned DNA
cataatgtaccatcgtgaaa

SEQ ID NO: 200 - DNA, 20 bp, Solanum tuberosum
  source 1..20: mol_type = unassigned DNA
cgaagcatacccatgagaat

SEQ ID NO: 201 - DNA, 20 bp, Solanum tuberosum
  source 1..20: mol_type = unassigned DNA
accttgcatgaggaggaaga

SEQ ID NO: 202 - DNA, 20 bp, Solanum tuberosum
  source 1..20: mol_type = unassigned DNA
tgatttggtattttcaggtg

SEQ ID NO: 203 - DNA, 20 bp, Solanum tuberosum
  source 1..20: mol_type = unassigned DNA
gtggatcgactggagcggtt

SEQ ID NO: 204 - DNA, 20 bp, Solanum tuberosum
  source 1..20: mol_type = unassigned DNA
tatctctctctttgctgtga

SEQ ID NO: 205 - DNA, 21 bp, Solanum tuberosum
  source 1..21: mol_type = unassigned DNA
cctcatatcaactacacacga

SEQ ID NO: 206 - DNA, 21 bp, Solanum tuberosum
  source 1..21: mol_type = unassigned DNA
tctcaagcgagctcttgttct

SEQ ID NO: 207 - DNA, 20 bp, Solanum tuberosum
  source 1..20: mol_type = unassigned DNA
ctattgagtttcgagtgtag

SEQ ID NO: 208 - DNA, 20 bp, Solanum tuberosum
  source 1..20: mol_type = unassigned DNA
gacgtggggtgatcaaagaa

SEQ ID NO: 209 - DNA, 20 bp, Solanum tuberosum
  source 1..20: mol_type = unassigned DNA
agtctcttctcgtgtgtagt

SEQ ID NO: 210 - DNA, 20 bp, Solanum tuberosum
  source 1..20: mol_type = unassigned DNA
atcaccccacgtccaaaaac

SEQ ID NO: 211 - DNA, 20 bp, Solanum tuberosum
  source 1..20: mol_type = unassigned DNA
gctttatatagttctggatg

SEQ ID NO: 212 - DNA, 20 bp, Solanum tuberosum
  source 1..20: mol_type = unassigned DNA
ctccataggccattgaaaga

SEQ ID NO: 213 - DNA, 20 bp, Solanum tuberosum
  source 1..20: mol_type = unassigned DNA
ctttcaggaactgaccattt

SEQ ID NO: 214 - DNA, 20 bp, Solanum tuberosum
  source 1..20: mol_type = unassigned DNA
gcactttaccaacagatctg

SEQ ID NO: 215 - DNA, 20 bp, Solanum tuberosum
  source 1..20: mol_type = unassigned DNA
atgtcaatgcgctgattttc

SEQ ID NO: 216 - DNA, 21 bp, Solanum tuberosum
  source 1..21: mol_type = unassigned DNA
cttatgagtgttagtggctgc

SEQ ID NO: 217 - DNA, 21 bp, Solanum tuberosum
  source 1..21: mol_type = unassigned DNA
ctcccagattccacataccgc

SEQ ID NO: 218 - DNA, 20 bp, Solanum tuberosum
  source 1..20: mol_type = unassigned DNA
tgaaccgatcgatgtagatc

SEQ ID NO: 219 - DNA, 20 bp, Solanum tuberosum
  source 1..20: mol_type = unassigned DNA
ttggccgctccagcattcaa

SEQ ID NO: 220 - DNA, 20 bp, Solanum tuberosum
  source 1..20: mol_type = unassigned DNA
gaaggcaccatgtacgtcca

SEQ ID NO: 221 - DNA, 20 bp, Solanum tuberosum
  source 1..20: mol_type = unassigned DNA
atatgttctgagatgtagag

SEQ ID NO: 222 - DNA, 20 bp, Solanum tuberosum
  source 1..20: mol_type = unassigned DNA
ttccagcagcagatatatgc

SEQ ID NO: 223 - DNA, 20 bp, Solanum tuberosum
  source 1..20: mol_type = unassigned DNA
ttatgctccatcactcattg

SEQ ID NO: 224 - DNA, 20 bp, Solanum tuberosum
  source 1..20: mol_type = unassigned DNA
acaattaactacattgatcg

SEQ ID NO: 225 - DNA, 20 bp, Solanum tuberosum
  source 1..20: mol_type = unassigned DNA
gggcaacttgatggacagac

SEQ ID NO: 226 - DNA, 20 bp, Solanum tuberosum
  source 1..20: mol_type = unassigned DNA
ttacgatgcgatcatcatgc

SEQ ID NO: 227 - DNA, 20 bp, Solanum tuberosum
  source 1..20: mol_type = unassigned DNA
ctcagggactgctgttctgc

SEQ ID NO: 228 - DNA, 20 bp, Solanum tuberosum
  source 1..20: mol_type = unassigned DNA
tctattttttattccagcag

SEQ ID NO: 229 - DNA, 20 bp, Solanum tuberosum
  source 1..20: mol_type = unassigned DNA
gaaactcctaacagaactct

SEQ ID NO: 230 - DNA, 20 bp, Zea mays
  source 1..20: mol_type = unassigned DNA
tggtggtgttggtggtggag

SEQ ID NO: 231 - DNA, 20 bp, Zea mays
  source 1..20: mol_type = unassigned DNA
tcaggagggtggcctcgccg

SEQ ID NO: 232 - DNA, 20 bp, Zea mays
  source 1..20: mol_type = unassigned DNA
ggcgttgagggtcacaccga

SEQ ID NO: 233 - DNA, 20 bp, Zea mays
  source 1..20: mol_type = unassigned DNA
tcggtgtgaccctcaacgcc

SEQ ID NO: 234 - DNA, 20 bp, Zea mays
  source 1..20: mol_type = unassigned DNA
caatggccgtaatatcacgg

SEQ ID NO: 235 - DNA, 20 bp, Zea mays
  source 1..20: mol_type = unassigned DNA
atcaggagggtggcctcgcc

SEQ ID NO: 236 - DNA, 20 bp, Zea mays
  source 1..20: mol_type = unassigned DNA
gtcggtgtgaccctcaacgc

SEQ ID NO: 237 - DNA, 20 bp, Zea mays
  source 1..20: mol_type = unassigned DNA
ctgcagagccccgagatgcc

SEQ ID NO: 238 - DNA, 20 bp, Zea mays
  source 1..20: mol_type = unassigned DNA
ctggatggtaccccaggacg

SEQ ID NO: 239 - DNA, 20 bp, Zea mays
  source 1..20: mol_type = unassigned DNA
gcaatggccgtaatatcacg

SEQ ID NO: 240 - DNA, 20 bp, Zea mays
  source 1..20: mol_type = unassigned DNA
gaggatggcaagctgaagac

SEQ ID NO: 241 - DNA, 20 bp, Zea mays
  source 1..20: mol_type = unassigned DNA
acgattgatgtgatgtcacg

SEQ ID NO: 242 - DNA, 20 bp, Zea mays
  source 1..20: mol_type = unassigned DNA
cctgactggtacccgaggac

SEQ ID NO: 243 - DNA, 20 bp, Zea mays
  source 1..20: mol_type = unassigned DNA
aagatgcttgaagtgaggac

SEQ ID NO: 244 - DNA, 20 bp, Zea mays
  source 1..20: mol_type = unassigned DNA
acagcaccattcaccagcac

SEQ ID NO: 245 - DNA, 20 bp, Zea mays
  source 1..20: mol_type = unassigned DNA
ccatcctccaaacaggttgc

SEQ ID NO: 246 - DNA, 20 bp, Zea mays
  source 1..20: mol_type = unassigned DNA
agcttgccatcctccaaaca

SEQ ID NO: 247 - DNA, 20 bp, Zea mays
  source 1..20: mol_type = unassigned DNA
ccgatctgtggatccaataa

SEQ ID NO: 248 - DNA, 20 bp, Zea mays
  source 1..20: mol_type = unassigned DNA
tcctcctctcaattgcctgc

SEQ ID NO: 249 - DNA, 20 bp, Zea mays
  source 1..20: mol_type = unassigned DNA
cggttgatcttcgagtggag

SEQ ID NO: 250 - DNA, 20 bp, Zea mays
  source 1..20: mol_type = unassigned DNA
atgatgtcgagcttgtcgag

SEQ ID NO: 251 - DNA, 20 bp, Zea mays
  source 1..20: mol_type = unassigned DNA
gaacccctcggtgcccatgg

SEQ ID NO: 252 - DNA, 20 bp, Zea mays
  source 1..20: mol_type = unassigned DNA
tcgagccgtttgcggttgat

SEQ ID NO: 253 - DNA, 20 bp, Zea mays
  source 1..20: mol_type = unassigned DNA
ctctcgtacacgatcaccac

SEQ ID NO: 254 - DNA, 20 bp, Zea mays
  source 1..20: mol_type = unassigned DNA
accttcctctcgtacacgat

SEQ ID NO: 255 - DNA, 20 bp, Zea mays
  source 1..20: mol_type = unassigned DNA
ctgtagagaagcttcacctt

SEQ ID NO: 256 - DNA, 20 bp, Zea mays
  source 1..20: mol_type = unassigned DNA
gagtggagcgtcgccgccat

SEQ ID NO: 257 - DNA, 20 bp, Zea mays
  source 1..20: mol_type = unassigned DNA
tcgcgcggaaggcgccgctc

SEQ ID NO: 258 - DNA, 20 bp, Zea mays
  source 1..20: mol_type = unassigned DNA
atcctgcggaggaggagcgc

SEQ ID NO: 259 - DNA, 20 bp, Zea mays
  source 1..20: mol_type = unassigned DNA
ttctgctgcaggagctgctg

SEQ ID NO: 260 - DNA, 20 bp, Zea mays
  source 1..20: mol_type = unassigned DNA
ctccagcctccgccgcaact

SEQ ID NO: 261 - DNA, 20 bp, Zea mays
  source 1..20: mol_type = unassigned DNA
gatcctgcggaggaggagcg

SEQ ID NO: 262 - DNA, 20 bp, Zea mays
  source 1..20: mol_type = unassigned DNA
gctccctgcagaagtagcag

SEQ ID NO: 263 - DNA, 20 bp, Zea mays
  source 1..20: mol_type = unassigned DNA
aagtgcagccggcacaatct

SEQ ID NO: 264 - DNA, 20 bp, Zea mays
  source 1..20: mol_type = unassigned DNA
gagcggcgccgctcgtcgcc

SEQ ID NO: 265 - DNA, 20 bp, Zea mays
  source 1..20: mol_type = unassigned DNA
aagagtatgcagatatcgtt

SEQ ID NO: 266 - DNA, 20 bp, Zea mays
  source 1..20: mol_type = unassigned DNA
gagcagtgcttgttctgctg

SEQ ID NO: 267 - DNA, 20 bp, Zea mays
  source 1..20: mol_type = unassigned DNA
gatgtattcgtactgaggcg

SEQ ID NO: 268 - DNA, 20 bp, Zea mays
  source 1..20: mol_type = unassigned DNA
attgcctcaatgtcaacagg

SEQ ID NO: 269 - DNA, 20 bp, Zea mays
  source 1..20: mol_type = unassigned DNA
cacatatcttggctccctgc

SEQ ID NO: 270 - DNA, 20 bp, Zea mays
  source 1..20: mol_type = unassigned DNA
gtgatgggctggataagatt

SEQ ID NO: 271 - DNA, 20 bp, Zea mays
  source 1..20: mol_type = unassigned DNA
tggagggcgaaaagaaagtg

SEQ ID NO: 272 - DNA, 20 bp, Zea mays
  source 1..20: mol_type = unassigned DNA
ccttctgtatattcaggctt

SEQ ID NO: 273 - DNA, 20 bp, Zea mays
  source 1..20: mol_type = unassigned DNA
tccattcctgttgacattga

SEQ ID NO: 274 - DNA, 20 bp, Zea mays
  source 1..20: mol_type = unassigned DNA
ttctcttgcagatgttgtta

SEQ ID NO: 275 - DNA, 20 bp, Zea mays
  source 1..20: mol_type = unassigned DNA
cagtgttccagcgtttggcc

SEQ ID NO: 276 - DNA, 20 bp, Zea mays
  source 1..20: mol_type = unassigned DNA
acagaacttgtcattggcca

SEQ ID NO: 277 - RNA, 20 nt, Zea mays
  source 1..20: mol_type = unassigned RNA
tggtggtgttggtggtggag

SEQ ID NO: 278 - RNA, 20 nt, Zea mays
  source 1..20: mol_type = unassigned RNA
tcaggagggtggcctcgccg

SEQ ID NO: 279 - RNA, 20 nt, Zea mays
  source 1..20: mol_type = unassigned RNA
ggcgttgagggtcacaccga

SEQ ID NO: 280 - RNA, 20 nt, Zea mays
  source 1..20: mol_type = unassigned RNA
tcggtgtgaccctcaacgcc

SEQ ID NO: 281 - RNA, 20 nt, Zea mays
  source 1..20: mol_type = unassigned RNA
caatggccgtaatatcacgg

SEQ ID NO: 282 - RNA, 20 nt, Zea mays
  source 1..20: mol_type = unassigned RNA
atcaggagggtggcctcgcc

SEQ ID NO: 283 - RNA, 20 nt, Zea mays
  source 1..20: mol_type = unassigned RNA
gtcggtgtgaccctcaacgc

SEQ ID NO: 284 - RNA, 20 nt, Zea mays
  source 1..20: mol_type = unassigned RNA
ctgcagagccccgagatgcc

SEQ ID NO: 285 - RNA, 20 nt, Zea mays
  source 1..20: mol_type = unassigned RNA
ctggatggtaccccaggacg

SEQ ID NO: 286 - RNA, 20 nt, Zea mays
  source 1..20: mol_type = unassigned RNA
gcaatggccgtaatatcacg

SEQ ID NO: 287 - RNA, 20 nt, Zea mays
  source 1..20: mol_type = unassigned RNA
gaggatggcaagctgaagac

SEQ ID NO: 288 - RNA, 20 nt, Zea mays
  source 1..20: mol_type = unassigned RNA
acgattgatgtgatgtcacg

SEQ ID NO: 289 - RNA, 20 nt, Zea mays
  source 1..20: mol_type = unassigned RNA
cctgactggtacccgaggac

SEQ ID NO: 290 - RNA, 20 nt, Zea mays
  source 1..20: mol_type = unassigned RNA
aagatgcttgaagtgaggac

SEQ ID NO: 291 - RNA, 20 nt, Zea mays
  source 1..20: mol_type = unassigned RNA
acagcaccattcaccagcac

SEQ ID NO: 292 - RNA, 20 nt, Zea mays
  source 1..20: mol_type = unassigned RNA
ccatcctccaaacaggttgc

SEQ ID NO: 293 - RNA, 20 nt, Zea mays
  source 1..20: mol_type = unassigned RNA
agcttgccatcctccaaaca

SEQ ID NO: 294 - RNA, 20 nt, Zea mays
  source 1..20: mol_type = unassigned RNA
ccgatctgtggatccaataa

SEQ ID NO: 295 - RNA, 20 nt, Zea mays
  source 1..20: mol_type = unassigned RNA
tcctcctctcaattgcctgc

SEQ ID NO: 296 - RNA, 20 nt, Zea mays
  source 1..20: mol_type = unassigned RNA
cggttgatcttcgagtggag

SEQ ID NO: 297 - RNA, 20 nt, Zea mays
  source 1..20: mol_type = unassigned RNA
atgatgtcgagcttgtcgag

SEQ ID NO: 298 - RNA, 20 nt, Zea mays
  source 1..20: mol_type = unassigned RNA
gaacccctcggtgcccatgg

SEQ ID NO: 299 - RNA, 20 nt, Zea mays
  source 1..20: mol_type = unassigned RNA
tcgagccgtttgcggttgat

SEQ ID NO: 300 - RNA, 20 nt, Zea mays
  source 1..20: mol_type = unassigned RNA
ctctcgtacacgatcaccac

SEQ ID NO: 301 - RNA, 20 nt, Zea mays
  source 1..20: mol_type = unassigned RNA
accttcctctcgtacacgat

SEQ ID NO: 302 - RNA, 20 nt, Zea mays
  source 1..20: mol_type = unassigned RNA
ctgtagagaagcttcacctt

SEQ ID NO: 303 - RNA, 20 nt, Zea mays
  source 1..20: mol_type = unassigned RNA
gagtggagcgtcgccgccat

SEQ ID NO: 304 - RNA, 20 nt, Zea mays
  source 1..20: mol_type = unassigned RNA
tcgcgcggaaggcgccgctc

SEQ ID NO: 305 - RNA, 20 nt, Zea mays
  source 1..20: mol_type = unassigned RNA
atcctgcggaggaggagcgc

SEQ ID NO: 306 - RNA, 20 nt, Zea mays
  source 1..20: mol_type = unassigned RNA
ttctgctgcaggagctgctg

SEQ ID NO: 307 - RNA, 20 nt, Zea mays
  source 1..20: mol_type = unassigned RNA
ctccagcctccgccgcaact

SEQ ID NO: 308 - RNA, 20 nt, Zea mays
  source 1..20: mol_type = unassigned RNA
gatcctgcggaggaggagcg

SEQ ID NO: 309 - RNA, 20 nt, Zea mays
  source 1..20: mol_type = unassigned RNA
gctccctgcagaagtagcag

SEQ ID NO: 310 - RNA, 20 nt, Zea mays
  source 1..20: mol_type = unassigned RNA
aagtgcagccggcacaatct

SEQ ID NO: 311 - RNA, 20 nt, Zea mays
  source 1..20: mol_type = unassigned RNA
gagcggcgccgctcgtcgcc

SEQ ID NO: 312 - RNA, 20 nt, Zea mays
  source 1..20: mol_type = unassigned RNA
aagagtatgcagatatcgtt

SEQ ID NO: 313 - RNA, 20 nt, Zea mays
  source 1..20: mol_type = unassigned RNA
gagcagtgcttgttctgctg

SEQ ID NO: 314 - RNA, 20 nt, Zea mays
  source 1..20: mol_type = unassigned RNA
gatgtattcgtactgaggcg

SEQ ID NO: 315 - RNA, 20 nt, Zea mays
  source 1..20: mol_type = unassigned RNA
attgcctcaatgtcaacagg

SEQ ID NO: 316 - RNA, 20 nt, Zea mays
  source 1..20: mol_type = unassigned RNA
cacatatcttggctccctgc

SEQ ID NO: 317 - RNA, 20 nt, Zea mays
  source 1..20: mol_type = unassigned RNA
gtgatgggctggataagatt

SEQ ID NO: 318 - RNA, 20 nt, Zea mays
  source 1..20: mol_type = unassigned RNA
tggagggcgaaaagaaagtg

SEQ ID NO: 319 - RNA, 20 nt, Zea mays
  source 1..20: mol_type = unassigned RNA
ccttctgtatattcaggctt

SEQ ID NO: 320 - RNA, 20 nt, Zea mays
  source 1..20: mol_type = unassigned RNA
tccattcctgttgacattga

SEQ ID NO: 321 - RNA, 20 nt, Zea mays
  source 1..20: mol_type = unassigned RNA
ttctcttgcagatgttgtta

SEQ ID NO: 322 - RNA, 20 nt, Zea mays
  source 1..20: mol_type = unassigned RNA
cagtgttccagcgtttggcc

SEQ ID NO: 323 - RNA, 20 nt, Zea mays
  source 1..20: mol_type = unassigned RNA
acagaacttgtcattggcca

SEQ ID NO: 324 - RNA, 20 nt, Arabidopsis thaliana
  source 1..20: mol_type = unassigned RNA
tttcagcgacgttatcaaaa

SEQ ID NO: 325 - RNA, 20 nt, Arabidopsis thaliana
  source 1..20: mol_type = unassigned RNA
agcgacgttatcaaaatgcc

SEQ ID NO: 326 - RNA, 20 nt, Arabidopsis thaliana
  source 1..20: mol_type = unassigned RNA
ccaaccgacggagacatggt

SEQ ID NO: 327 - RNA, 20 nt, Arabidopsis thaliana
  source 1..20: mol_type = unassigned RNA
acttgacgagccagattcac

SEQ ID NO: 328 - RNA, 20 nt, Arabidopsis thaliana
  source 1..20: mol_type = unassigned RNA
cttgacgagccagattcacg

SEQ ID NO: 329 - RNA, 20 nt, Arabidopsis thaliana
  source 1..20: mol_type = unassigned RNA
attgaatctccggtgaatcc

SEQ ID NO: 330 - RNA, 20 nt, Arabidopsis thaliana
  source 1..20: mol_type = unassigned RNA
actacatggaggcctggtaa

SEQ ID NO: 331 - RNA, 20 nt, Arabidopsis thaliana
  source 1..20: mol_type = unassigned RNA
gattgccacagggaagagag

SEQ ID NO: 332 - RNA, 20 nt, Arabidopsis thaliana
  source 1..20: mol_type = unassigned RNA
ttgccttcttggtatccaag

SEQ ID NO: 333 - RNA, 20 nt, Arabidopsis thaliana
  source 1..20: mol_type = unassigned RNA
tgttctcctgacagtggtac

SEQ ID NO: 334 - RNA, 20 nt, Arabidopsis thaliana
  source 1..20: mol_type = unassigned RNA
gcagttgatggtgggcaact

SEQ ID NO: 335 - RNA, 20 nt, Arabidopsis thaliana
  source 1..20: mol_type = unassigned RNA
aactttagcagttgatggtg

SEQ ID NO: 336 - RNA, 20 nt, Arabidopsis thaliana
  source 1..20: mol_type = unassigned RNA
ttaatattcatcttcatcac

SEQ ID NO: 337 - RNA, 20 nt, Arabidopsis thaliana
  source 1..20: mol_type = unassigned RNA
tgtctttcctcctaatttaa

SEQ ID NO: 338 - RNA, 20 nt, Arabidopsis thaliana
  source 1..20: mol_type = unassigned RNA
gaattagaaggagatcagca

SEQ ID NO: 339 - RNA, 20 nt, Arabidopsis thaliana
  source 1..20: mol_type = unassigned RNA
ctcagatgactcagagttct

SEQ ID NO: 340 - RNA, 20 nt, Arabidopsis thaliana
  source 1..20: mol_type = unassigned RNA
cctttttgcacagagattta

SEQ ID NO: 341 - RNA, 20 nt, Arabidopsis thaliana
  source 1..20: mol_type = unassigned RNA
tgcacagagatttagttctc

SEQ ID NO: 342 - RNA, 20 nt, Arabidopsis thaliana
  source 1..20: mol_type = unassigned RNA
ctgagaactaaatctctgtg

SEQ ID NO: 343 - RNA, 20 nt, Arabidopsis thaliana
  source 1..20: mol_type = unassigned RNA
gttctcaggaaagagatctc

SEQ ID NO: 344 - RNA, 20 nt, Arabidopsis thaliana
  source 1..20: mol_type = unassigned RNA
aagaaggttagtagttgctt

SEQ ID NO: 345 - RNA, 20 nt, Arabidopsis thaliana
  source 1..20: mol_type = unassigned RNA
tgattgattggaggtaagca

SEQ ID NO: 346 - RNA, 20 nt, Arabidopsis thaliana
  source 1..20: mol_type = unassigned RNA
cttcatttgtcacctagtag

SEQ ID NO: 347 - RNA, 20 nt, Arabidopsis thaliana
  source 1..20: mol_type = unassigned RNA
ggatgatggagacttctttg

SEQ ID NO: 348 - RNA, 20 nt, Arabidopsis thaliana
  source 1..20: mol_type = unassigned RNA
agcaggtttggaatgatgtt

SEQ ID NO: 349 - RNA, 20 nt, Arabidopsis thaliana
  source 1..20: mol_type = unassigned RNA
gttcctcgattggcttgcat

SEQ ID NO: 350 - RNA, 20 nt, Arabidopsis thaliana
  source 1..20: mol_type = unassigned RNA
aagaaatacttctttagttc

SEQ ID NO: 351 - RNA, 20 nt, Arabidopsis thaliana
  source 1..20: mol_type = unassigned RNA
ttctgtttactgcagctgaa

SEQ ID NO: 352 - RNA, 20 nt, Arabidopsis thaliana
  source 1..20: mol_type = unassigned RNA
gaatcggcttgttcctttct

SEQ ID NO: 353 - RNA, 20 nt, Arabidopsis thaliana
  source 1..20: mol_type = unassigned RNA
ttctcaccagcttctagctc

SEQ ID NO: 354 - RNA, 20 nt, Arabidopsis thaliana
  source 1..20: mol_type = unassigned RNA
actcaccatatctgacccaa

SEQ ID NO: 355 - RNA, 20 nt, Arabidopsis thaliana
  source 1..20: mol_type = unassigned RNA
actgatagaatttgactcac

SEQ ID NO: 356 - RNA, 20 nt, Arabidopsis thaliana
  source 1..20: mol_type = unassigned RNA
ttccggttgatcttcgcgtg

SEQ ID NO: 357 - RNA, 20 nt, Arabidopsis thaliana
  source 1..20: mol_type = unassigned RNA
aatgatatcgagcttatcta

SEQ ID NO: 358 - RNA, 20 nt, Arabidopsis thaliana
  source 1..20: mol_type = unassigned RNA
ctcttacagcgaagagattt

SEQ ID NO: 359 - RNA, 20 nt, Arabidopsis thaliana
  source 1..20: mol_type = unassigned RNA
gaatccgtcggttccgatgg

SEQ ID NO: 360 - RNA, 20 nt, Arabidopsis thaliana
  source 1..20: mol_type = unassigned RNA
gtttattcgataggtggtgt

SEQ ID NO: 361 - RNA, 20 nt, Arabidopsis thaliana
  source 1..20: mol_type = unassigned RNA
ttcgataggtggtgttgtga

SEQ ID NO: 362 - RNA, 20 nt, Arabidopsis thaliana
  source 1..20: mol_type = unassigned RNA
ctctcataaacaatcacaac

SEQ ID NO: 363 - RNA, 20 nt, Arabidopsis thaliana
  source 1..20: mol_type = unassigned RNA
tgagaggaaagtgaagctcc

SEQ ID NO: 364 - RNA, 20 nt, Arabidopsis thaliana
  source 1..20: mol_type = unassigned RNA
aatcagaaaattaccgaata

SEQ ID NO: 365 - RNA, 20 nt, Arabidopsis thaliana
  source 1..20: mol_type = unassigned RNA
cgtcaatatttcagatgatg

SEQ ID NO: 366 - RNA, 20 nt, Arabidopsis thaliana
  source 1..20: mol_type = unassigned RNA
agatgatgttaatcgctttc

SEQ ID NO: 367 - RNA, 20 nt, Arabidopsis thaliana
  source 1..20: mol_type = unassigned RNA
attaccagaaagcgattaac

SEQ ID NO: 368 - RNA, 20 nt, Arabidopsis thaliana
  source 1..20: mol_type = unassigned RNA
tctcaggttgaaattaatgg

SEQ ID NO: 369 - RNA, 20 nt, Arabidopsis thaliana
  source 1..20: mol_type = unassigned RNA
tctcaagatactcaccttga

SEQ ID NO: 370 - RNA, 20 nt, Arabidopsis thaliana
  source 1..20: mol_type = unassigned RNA
tgtaacattctcagctgatg

SEQ ID NO: 371 - RNA, 20 nt, Arabidopsis thaliana
  source 1..20: mol_type = unassigned RNA
atcatctccttcgatgtcaa

SEQ ID NO: 372 - RNA, 20 nt, Arabidopsis thaliana
  source 1..20: mol_type = unassigned RNA
agaatcgactaatttgcttc

SEQ ID NO: 373 - RNA, 20 nt, Arabidopsis thaliana
  source 1..20: mol_type = unassigned RNA
aagcaaattagtcgattctg

SEQ ID NO: 374 - RNA, 20 nt, Arabidopsis thaliana
  source 1..20: mol_type = unassigned RNA
atcctttgaagcaaattagt

SEQ ID NO: 375 - RNA, 20 nt, Arabidopsis thaliana
  source 1..20: mol_type = unassigned RNA
gagcagattttactcaatcg

SEQ ID NO: 376 - RNA, 20 nt, Arabidopsis thaliana
  source 1..20: mol_type = unassigned RNA
ctcaatcggtcgttgtagat

SEQ ID NO: 377 - RNA, 20 nt, Arabidopsis thaliana
  source 1..20: mol_type = unassigned RNA
gagatcttccttcagccaga

SEQ ID NO: 378 - RNA, 20 nt, Arabidopsis thaliana
  source 1..20: mol_type = unassigned RNA
catcaatcaattcaggaact

SEQ ID NO: 379 - RNA, 20 nt, Arabidopsis thaliana
  source 1..20: mol_type = unassigned RNA
attctgctgtctatagctta

SEQ ID NO: 380 - RNA, 20 nt, Arabidopsis thaliana
  source 1..20: mol_type = unassigned RNA
gtttatcagagctgcataag

SEQ ID NO: 381 - RNA, 20 nt, Arabidopsis thaliana
  source 1..20: mol_type = unassigned RNA
ctgaccctttggtttatcag

SEQ ID NO: 382 - RNA, 20 nt, Arabidopsis thaliana
  source 1..20: mol_type = unassigned RNA
cactcttaagaaggaaccac

SEQ ID NO: 383 - RNA, 20 nt, Arabidopsis thaliana
  source 1..20: mol_type = unassigned RNA
tggttccttcttaagagtga

SEQ ID NO: 384 - RNA, 20 nt, Arabidopsis thaliana
  source 1..20: mol_type = unassigned RNA
cgaagcatgtctattttctt

SEQ ID NO: 385 - RNA, 20 nt, Arabidopsis thaliana
  source 1..20: mol_type = unassigned RNA
accaacccatcaccaaccta

SEQ ID NO: 386 - DNA, 20 bp, Arabidopsis thaliana
  source 1..20: mol_type = unassigned DNA
tttcagcgacgttatcaaaa

SEQ ID NO: 387 - DNA, 20 bp, Arabidopsis thaliana
  source 1..20: mol_type = unassigned DNA
agcgacgttatcaaaatgcc

SEQ ID NO: 388 - DNA, 20 bp, Arabidopsis thaliana
  source 1..20: mol_type = unassigned DNA
ccaaccgacggagacatggt

SEQ ID NO: 389 - DNA, 20 bp, Arabidopsis thaliana
  source 1..20: mol_type = unassigned DNA
acttgacgagccagattcac

SEQ ID NO: 390 - DNA, 20 bp, Arabidopsis thaliana
  source 1..20: mol_type = unassigned DNA
cttgacgagccagattcacg

SEQ ID NO: 391 - DNA, 20 bp, Arabidopsis thaliana
  source 1..20: mol_type = unassigned DNA
attgaatctccggtgaatcc

SEQ ID NO: 392 - DNA, 20 bp, Arabidopsis thaliana
  source 1..20: mol_type = unassigned DNA
actacatggaggcctggtaa

SEQ ID NO: 393 - DNA, 20 bp, Arabidopsis thaliana
  source 1..20: mol_type = unassigned DNA
gattgccacagggaagagag

SEQ ID NO: 394 - DNA, 20 bp, Arabidopsis thaliana
  source 1..20: mol_type = unassigned DNA
ttgccttcttggtatccaag

SEQ ID NO: 395 - DNA, 20 bp, Arabidopsis thaliana
  source 1..20: mol_type = unassigned DNA
tgttctcctgacagtggtac

SEQ ID NO: 396 - DNA, 20 bp, Arabidopsis thaliana
  source 1..20: mol_type = unassigned DNA
gcagttgatggtgggcaact

SEQ ID NO: 397 - DNA, 20 bp, Arabidopsis thaliana
  source 1..20: mol_type = unassigned DNA
aactttagcagttgatggtg

SEQ ID NO: 398 - DNA, 20 bp, Arabidopsis thaliana
  source 1..20: mol_type = unassigned DNA
ttaatattcatcttcatcac

SEQ ID NO: 399 - DNA, 20 bp, Arabidopsis thaliana
  source 1..20: mol_type = unassigned DNA
tgtctttcctcctaatttaa

SEQ ID NO: 400 - DNA, 20 bp, Arabidopsis thaliana
  source 1..20: mol_type = unassigned DNA
gaattagaaggagatcagca

SEQ ID NO: 401 - DNA, 20 bp, Arabidopsis thaliana
  source 1..20: mol_type = unassigned DNA
ctcagatgactcagagttct

SEQ ID NO: 402 - DNA, 20 bp, Arabidopsis thaliana
  source 1..20: mol_type = unassigned DNA
cctttttgcacagagattta

SEQ ID NO: 403 - DNA, 20 bp, Arabidopsis thaliana
  source 1..20: mol_type = unassigned DNA
tgcacagagatttagttctc

SEQ ID NO: 404 - DNA, 20 bp, Arabidopsis thaliana
  source 1..20: mol_type = unassigned DNA
ctgagaactaaatctctgtg

SEQ ID NO: 405 - DNA, 20 bp, Arabidopsis thaliana
  source 1..20: mol_type = unassigned DNA
gttctcaggaaagagatctc

SEQ ID NO: 406 - DNA, 20 bp, Arabidopsis thaliana
  source 1..20: mol_type = unassigned DNA
aagaaggttagtagttgctt

SEQ ID NO: 407 - DNA, 20 bp, Arabidopsis thaliana
  source 1..20: mol_type = unassigned DNA
tgattgattggaggtaagca

SEQ ID NO: 408 - DNA, 20 bp, Arabidopsis thaliana
  source 1..20: mol_type = unassigned DNA
cttcatttgtcacctagtag

SEQ ID NO: 409 - DNA, 20 bp, Arabidopsis thaliana
  source 1..20: mol_type = unassigned DNA
ggatgatggagacttctttg

SEQ ID NO: 410 - DNA, 20 bp, Arabidopsis thaliana
  source 1..20: mol_type = unassigned DNA
agcaggtttggaatgatgtt

SEQ ID NO: 411 - DNA, 20 bp, Arabidopsis thaliana
  source 1..20: mol_type = unassigned DNA
gttcctcgattggcttgcat

SEQ ID NO: 412 - DNA, 20 bp, Arabidopsis thaliana
  source 1..20: mol_type = unassigned DNA
aagaaatacttctttagttc

SEQ ID NO: 413 - DNA, 20 bp, Arabidopsis thaliana
  source 1..20: mol_type = unassigned DNA
ttctgtttactgcagctgaa

SEQ ID NO: 414 - DNA, 20 bp, Arabidopsis thaliana
  source 1..20: mol_type = unassigned DNA
gaatcggcttgttcctttct

SEQ ID NO: 415 - DNA, 20 bp, Arabidopsis thaliana
  source 1..20: mol_type = unassigned DNA
ttctcaccagcttctagctc

SEQ ID NO: 416 - DNA, 20 bp, Arabidopsis thaliana
  source 1..20: mol_type = unassigned DNA
actcaccatatctgacccaa

SEQ ID NO: 417 - DNA, 20 bp, Arabidopsis thaliana
  source 1..20: mol_type = unassigned DNA
actgatagaatttgactcac

SEQ ID NO: 418 - DNA, 20 bp, Arabidopsis thaliana
  source 1..20: mol_type = unassigned DNA
ttccggttgatcttcgcgtg

SEQ ID NO: 419 - DNA, 20 bp, Arabidopsis thaliana
  source 1..20: mol_type = unassigned DNA
aatgatatcgagcttatcta

SEQ ID NO: 420 - DNA, 20 bp, Arabidopsis thaliana
  source 1..20: mol_type = unassigned DNA
ctcttacagcgaagagattt

SEQ ID NO: 421 - DNA, 20 bp, Arabidopsis thaliana
  source 1..20: mol_type = unassigned DNA
gaatccgtcggttccgatgg

SEQ ID NO: 422 - DNA, 20 bp, Arabidopsis thaliana
  source 1..20: mol_type = unassigned DNA
gtttattcgataggtggtgt

SEQ ID NO: 423 - DNA, 20 bp, Arabidopsis thaliana
  source 1..20: mol_type = unassigned DNA
ttcgataggtggtgttgtga

SEQ ID NO: 424 - DNA, 20 bp, Arabidopsis thaliana
  source 1..20: mol_type = unassigned DNA
ctctcataaacaatcacaac

SEQ ID NO: 425 - DNA, 20 bp, Arabidopsis thaliana
  source 1..20: mol_type = unassigned DNA
tgagaggaaagtgaagctcc

SEQ ID NO: 426 - DNA, 20 bp, Arabidopsis thaliana
  source 1..20: mol_type = unassigned DNA
aatcagaaaattaccgaata

SEQ ID NO: 427 - DNA, 20 bp, Arabidopsis thaliana
  source 1..20: mol_type = unassigned DNA
cgtcaatatttcagatgatg

SEQ ID NO: 428 - DNA, 20 bp, Arabidopsis thaliana
  source 1..20: mol_type = unassigned DNA
agatgatgttaatcgctttc

SEQ ID NO: 429 - DNA, 20 bp, Arabidopsis thaliana
  source 1..20: mol_type = unassigned DNA
attaccagaaagcgattaac

SEQ ID NO: 430 - DNA, 20 bp, Arabidopsis thaliana
  source 1..20: mol_type = unassigned DNA
tctcaggttgaaattaatgg

SEQ ID NO: 431 - DNA, 20 bp, Arabidopsis thaliana
  source 1..20: mol_type = unassigned DNA
tctcaagatactcaccttga

SEQ ID NO: 432 - DNA, 20 bp, Arabidopsis thaliana
  source 1..20: mol_type = unassigned DNA
tgtaacattctcagctgatg

SEQ ID NO: 433 - DNA, 20 bp, Arabidopsis thaliana
  source 1..20: mol_type = unassigned DNA
atcatctccttcgatgtcaa

SEQ ID NO: 434 - DNA, 20 bp, Arabidopsis thaliana
  source 1..20: mol_type = unassigned DNA
agaatcgactaatttgcttc

SEQ ID NO: 435 - DNA, 20 bp, Arabidopsis thaliana
  source 1..20: mol_type = unassigned DNA
aagcaaattagtcgattctg

SEQ ID NO: 436 - DNA, 20 bp, Arabidopsis thaliana
  source 1..20: mol_type = unassigned DNA
atcctttgaagcaaattagt

SEQ ID NO: 437 - DNA, 20 bp, Arabidopsis thaliana
  source 1..20: mol_type = unassigned DNA
gagcagattttactcaatcg

SEQ ID NO: 438 - DNA, 20 bp, Arabidopsis thaliana
  source 1..20: mol_type = unassigned DNA
ctcaatcggtcgttgtagat

SEQ ID NO: 439 - DNA, 20 bp, Arabidopsis thaliana
  source 1..20: mol_type = unassigned DNA
gagatcttccttcagccaga

SEQ ID NO: 440 - DNA, 20 bp, Arabidopsis thaliana
  source 1..20: mol_type = unassigned DNA
catcaatcaattcaggaact

SEQ ID NO: 441 - DNA, 20 bp, Arabidopsis thaliana
  source 1..20: mol_type = unassigned DNA
attctgctgtctatagctta

SEQ ID NO: 442 - DNA, 20 bp, Arabidopsis thaliana
  source 1..20: mol_type = unassigned DNA
gtttatcagagctgcataag

SEQ ID NO: 443 - DNA, 20 bp, Arabidopsis thaliana
  source 1..20: mol_type = unassigned DNA
ctgaccctttggtttatcag

SEQ ID NO: 444 - DNA, 20 bp, Arabidopsis thaliana
  source 1..20: mol_type = unassigned DNA
cactcttaagaaggaaccac

SEQ ID NO: 445 - DNA, 20 bp, Arabidopsis thaliana
  source 1..20: mol_type = unassigned DNA
tggttccttcttaagagtga

SEQ ID NO: 446 - DNA, 20 bp, Arabidopsis thaliana
  source 1..20: mol_type = unassigned DNA
cgaagcatgtctattttctt

SEQ ID NO: 447 - DNA, 20 bp, Arabidopsis thaliana
  source 1..20: mol_type = unassigned DNA
accaacccatcaccaaccta

SEQ ID NO: 448 - DNA, 412 bp, Zea mays
  source 1..412: mol_type = unassigned DNA
tactaactgctagctgcaggcaattgagaggaggagaagtcgtctgcagaatgctgcagctcagcagcagatccagtggacagaagacccttcccgatctgtggatccaataactccagtacaggcagagcagggtggtgtgccaacaactgtggatggtcaaggtgttggaagccctgcaacctgtttggaggatggcagtcctatccatcatctgactgctccttgcaggccactccatccaaaccaaacgatccagctctcgcagatctcgtggagaagaagctgtccagctcgatagagcagatcgagaagatggtgcggcgaaacctgaagagaacttcgaaggccgctcagccttccaagaggaccatccagaggcgcgtcctgatgtccatgcgatgagctgaga

SEQ ID NO: 449 - DNA, 444 bp, Zea mays
  source 1..444: mol_type = unassigned DNA
ctggaaatttgctgatgtactaactgctagttgcaggcaattgagaggaggagaagtcgtctgcagaatgctgcagctcagcagcagatcctgtggacagaagacccttcccgatctgtggatccaataactccagtacaggcagagcatggtggtgtgccaacaactgaggatggtcaaggtgttggaagccctgcaacctgtttggaggatggcaagctgaaggtctgactgctccttgcaggccactccatccaaaccaaacgatccagctcgcgcagatctcgtggagaagaagttgtccagctcgatagagcagatcgagaagatggtgcggcgaaacctgaagagaacttcgaaggccgctcagccttccaagaggaccatccagaggcgcgtcctgatgtccatgcgatgagctgagaaagctatctgctctgccat

SEQ ID NO: 450 - DNA, 400 bp, Zea mays
  source 1..400: mol_type = unassigned DNA
tggcggcgacgctccactcgaagatcaaccgcaaacggctcgacaagctcgacatcatcaaaatctggtggggaaactggtcccagggattcgagttccgcacggttttgactgctatttttgtgttttttttatttgtttttgctgacttttgtttgtgtgtttcacttttagtgaggaaattttgaacccctcggtgcggctctctggaatcctcatgggtgagttcgattttgcttgcgccacccaaaactgttcctcccattttttggatagtttttttttacgttccaatttccatggtcgaaaattcgaaatgcgtgagcaggtggcgtggtgatcgtgtacgagaggaaggtgaagcttctctacagtaagtttcttcctccctacatcctac

SEQ ID NO: 451 - DNA, 400 bp, Zea mays
  source 1..400: mol_type = unassigned DNA
aacattgcacctactgagcacagacctacgaagatggaggggtacatgtagtagatgtccctcttggagcagtgcttgttctgctgcaggagctgctgcaccacgaacagcaccctcaggagcacatctacgcatgaacaacgcatcagaaagtaattagtgtgatcacataaatgcaaagatttagatgtattcgtactgaggtgtggtagtccttgtggagcagagagaggacgtccgtgccgacggggatgtcgtagctgcaggcactgcaatcacgcacgagcgcgcgcccagggtctacggatgtcacacatttgggtgaaaattgttcgagggatgtgacggtgaattggtcgggaaggagatgatcccgggagcgggcgcggaagctaaacca

SEQ ID NO: 452 - DNA, 203 bp, Arabidopsis thaliana
  source 1..203: mol_type = unassigned DNA
gacactcttgaactacaggctattgagagaagaagaggagctgggactggaggagacgatggccgagttattgagatcccaactcatcgacaagttggtgttcctgtcaggagaacacaaatgctcgatggtcactcctggaccatctgtgggattcaagcgtagttgcccaccatcaactgctaaagttcaaaagatgttgc

SEQ ID NO: 453 - DNA, 216 bp, Arabidopsis thaliana
  source 1..216: mol_type = unassigned DNA
cactcttgaactacaggctattgagagaagaagaggagctgggactggaggagacgatggccgagttattgagatcccaactcatcgacaagttggtgttcttgaatctcgatggtcactcctggaccatctgtgggattcaagcgtagttgcccaccatcaactgctaaagttcaaaagatgttgcttgacatcactaaagagatagctgaggaa

SEQ ID NO: 454 - DNA, 220 bp, Arabidopsis thaliana
  source 1..220: mol_type = unassigned DNA
ttttgctcatcggattttttttttcctcagttcttcacattctgggttcttcaatgttttgtttattcgataggtggtgttgtgattgtttatgagaggaaagttcggtaattttctgattcaaataatttttgaattttgggatttaagtttacactctcctctttttctgatgagactctgctctttttctgggttttattttccgtcaatatttcag

SEQ ID NO: 455 - DNA, 246 bp, Arabidopsis thaliana
  source 1..246: mol_type = unassigned DNA
tcttatgggtatgcttttgctcatcggattttttttttcctcagttcttcacattctgggttcttcaatgttttgtttattcgataggtggtgttgtgattgtttatgagaggaaagtattcggtaattttctgattcaaataatttttgaattttgggatttaagtttacactctcctctttttctgatgagactctgctctttttctgggttttattttccgtcaatatttcagatgatgttaa

SEQ ID NO: 456 - DNA, 242 bp, Arabidopsis thaliana
  source 1..242: mol_type = unassigned DNA
acatatctgaaagtaaaaacaccgcaatgtgatgctatattaagataacactaatgtacttgtagaattatctgaaacctgactcaccgattctgtaggtttgtggttccttcttaagacctgaccctttggtttatcagagccgcataagctatagacagcagaataaaatggtaaattcatgaggtcttagttttcaatactcgttgacgttgtgcttaaatccttcaaaaatgtctcga

SEQ ID NO: 457 - DNA, 227 bp, Arabidopsis thaliana
  source 1..227: mol_type = unassigned DNA
cttagcaacatatctgaaagtaaaaacaccgcaatgtgatgctatattaagataacactaatgtacttgtagaattatctgaaacctgactcaccgattctgtaggtttgtggttccttcttattcctgaccctttggtttatcagagccgcataagctatagacagcagaataaaatggtaaattcatgaggtcttagttttcaatactcgttgacgttgtgctta

SEQ ID NO: 458 - DNA, 263 bp, Arabidopsis thaliana
  source 1..263: mol_type = unassigned DNA
ttgaatctccggtgaatccagatattgggtctattggtggaacgggcgggcttgtgagaggcaatttcactacatggaggcctggtaatggcagaggtggtcacactccatttagattgccacagggaatatgcccatagtgaccgctaggcgtggaagaggtggtggtttgttgccttcttggtatccaagaacacctctacgcgacattactcatattgtgcgggtaaattgacaagctcttttgtgttttcctgttgatt

SEQ ID NO: 459 - DNA, 217 bp, Arabidopsis thaliana
  source 1..217: mol_type = unassigned DNA
aagatctagaagcgaccaactgactatcttctcgtttatgattgattggaggtaagcaactactaaccttcttcaaaatgcccatagtgaccgctaggcgtggaagaggtggtggtttgttgccttcttggtatccaagaacacctctacgcgacattactcatattgtgcgggtaaattgacaagctcttttgtgttttcctgttgattcttcttt

SEQ ID NO: 460 - DNA, 352 bp, Arabidopsis thaliana
  source 1..352: mol_type = unassigned DNA
ttttgttggttactttactcttacagcgaagagattttgaatccgtcggttccgatggctcttagactctccgggattcttatgggtatgcttttgctcatcggattttttttttcctcagttcttcacattctgggttcttcaatgttttgtttattcgataggtggtgttgtgattgtttatgagaggaaagtcggtaattttctgattcaaatcatttttgaattttgggatttaagtttacactctcctctttttctgatgagactctgctctttttctgggttttattttccgtcaatatttcagatgatgttaatcgctttctggtaatcaaatcttctccgatcc

SEQ ID NO: 461 - DNA, 251 bp, Arabidopsis thaliana
  source 1..251: mol_type = unassigned DNA
aactcctcattaacgatcaggtttgctacttagctatagatcgtctcttcttctgattatcgatttcatatttctgtgcttgcttttcctttttgcacagagatttagttctcaggaaagtcggtaattttctgattcaaatcatttttgaattttgggatttaagtttacactctcctctttttctgatgagactctgctctttttctgggttttattttccgtcaatatttcagatgatgttaatcgct

SEQ ID NO: 462 - DNA, 390 bp, Arabidopsis thaliana
  source 1..390: mol_type = unassigned DNA
ccttctctcagggaccttcttcgtctcagcgtggttgtggtggtttttctcagatgactcagagttctatcgacgaactcctcattaacgatcaggtttgctacttagctatagatcgtctcttcttctgattatcgatttcatatttctgtgcttgcttttcctttttgcacagagatttagttctcaggaaattgaagaaggttagtagttgcttacctccaatcaatcataaacgagaagatagtcagttggtcgcttctagatcttcaagtggtctttcgagaagatggagttctgcttcaattggagagtctaagtgtaagtatgctctttctaaattcacaaattagcgtggggatatgttcctaatttgattcaacttttctt

SEQ ID NO: 463 - DNA, 252 bp, Arabidopsis thaliana
  source 1..252: mol_type = unassigned DNA
caggaaaacacaaaagagcttgtcaatttacccgcacaatatgagtaatgtcgcgtagaggtgttcttggataccaagaaggcaacaaaccaccacctcttccacgcctagcggtcactatgggcattttgaagaaggttagtagttgcttacctccaatcaatcataaacgagaagatagtcagttggtcgcttctagatcttcaagtggtctttcgagaagatggagttctgcttcaattggagagtcta

SEQ ID NO: 464 - DNA, 263 bp, Solanum tuberosum
  source 1..263: mol_type = unassigned DNA
tcagggaggatagcaacttttaattatcattcctacccttttcactatcatatcatttcttgtccaactatgtgatgaagatcagcagatacaatatacaagtattgtttactctccctagactcacctacgtgcatggcattccctcttcagggatatagcttccttgccagtagacaaaccagagctgcaagagctacataaattaaacaaagaccattaatactaccacgacatttggatggtctttattagccattcaa

SEQ ID NO: 465 - DNA, 266 bp, Solanum tuberosum
  source 1..266: mol_type = unassigned DNA
cagttcaacgtagtagtggaagaaaacgcatcaccagatgttaattatattgagtttactaagctttttcctattgagtttcgagtgtagagtggccggtgagtctagggagagtaaataatacttgtatattttatctgctgatcttcatcacatagttggacaagaaatgacatgatagtgaagagggtaggaatgataattaaatgttgctatcctccctgaaaagtgacaaaattaccagggtaactatttccccgttaagt

SEQ ID NO: 466 - DNA, 219 bp, Solanum tuberosum
  source 1..219: mol_type = unassigned DNA
tgttctgtattcgttgaagttccttcataggattttttgtatacgttaagttttcacatttcaatttggctacgaagaatgtttaaattgatgttgtttatatctctctctttgctgtgaatacgtgcatggcattccctcttcagggatatagcttccttgccagtcgacaaaccagagctgcaagagctacataaattaaacaaagaccattaatac

SEQ ID NO: 467 - skipped

SEQ ID NO: 468 - DNA, 224 bp, Solanum tuberosum
  source 1..224: mol_type = unassigned DNA
gtatctaatgtaatgtattcgttgaagttccttcataggattttcggtatacgttaagttttcacatttcaatttgactacgaagtatgtttaaattgatgttgtttatatctcggccactctacactcgaaactcaataggaaaaagcttagtaaactcaatattattaacatctggtgatgcgttttcttccactactacgttgaactgcctttattccttg

SEQ ID NO: 469 - DNA, 202 bp, Solanum tuberosum
  source 1..202: mol_type = unassigned DNA
gttgaagttccttcataggattttcggtatacgttaagttttcacatttcaatttgactacgaagtatgtttaaattgatgttgtttatatctctctttgctgggaaaaagcttagtaaactcaatattattaacatctggtgatgcgttttcttccactactacgttgaactgcctttattccttgttgattttgagtaat

SEQ ID NO: 470 - DNA, 238 bp, Solanum tuberosum
  source 1..238: mol_type = unassigned DNA
catttttcagggaggatagcaacttttaattatcattcctacccccttcactatcatatcatatcttgtccaactatgtgatgaagatcagtagataaaatatacaagtattgtttactctccctagactcacctacgtgcatggcattccctcttcagggatatagcttccttgccggtcgacaaaccagagctgcaagagctacataaattaaacaaagaccattaatactaccac

SEQ ID NO: 471 - DNA, 245 bp, Solanum tuberosum
  source 1..245: mol_type = unassigned DNA
cttttcagggaggatagcaacatttaattatcattcctaccctcttcactatcatatcatttcttgtccaactatgtgatgaagatcagcagataaaatatacaagtattatttactctccctagactctacgtgcttggcattccctcttcagggatatagcttccttgccagtcgacaaaccagagctgcaagagctacataaattaaacaaagaccattaatactaccacgacatttggatg

SEQ ID NO: 472-473 - skipped (2 entries)

SEQ ID NO: 474 - DNA, 242 bp, Solanum tuberosum
  source 1..242: mol_type = unassigned DNA
atctaatgttctgtattcgttgaagttccttcataggattttttgtatacgttaagttttcacatttcaatttggctacgaagaatgtttaaattgatgttgtttatatctctctctttgctgtgaggccactctacactcgaaactcaataggaaaaagcttagtaaactcaatataattaacatctggtgatgcgttttcttccactactacgttgaactgcctttattccttgttgatt

SEQ ID NO: 475 - DNA, 292 bp, Solanum tuberosum
  source 1..292: mol_type = unassigned DNA
gtcttttgcttttctgtatctaatgtaatgtattcgttgaagttccttcataggattttcggtatacgttaagttttcacatttcaatttgactacgaagaatgtttaaattgatgctgtttatatctctctctttgctgtgaaggccactctacactcgaaactcaataggaaaaagcttagtaaactcaatattattaacatctggtgaggcgttgtcttccactactacgttgaactgcctttattccttgttgattttgagtaatcatcttctgcaatttcttttgtc

SEQ ID NO: 476 - DNA, 234 bp, Solanum tuberosum
  source 1..234: mol_type = unassigned DNA
ttaattatcattcctaccctcttcactatcatgtcatttcttgtccaactatgtgatgaagatcagcagataaaatatacaagtattatttactctccctagactcaacgtgcatggcattccctcttcagggatatagcttccttgccagtcgacaaaccagagctgcaagagctacataaattaaacaaagaccattaatactaccacgacatttggatggtctttattagc

SEQ ID NO: 477 - DNA, 520 bp, Solanum tuberosum
  source 1..520: mol_type = unassigned DNA
ccccaccccactccaccttccccccatggtgcatcctgtgtaacttcaaggaaagggaggctcatataggaaaacagaagaacagaaaagtcaacttaacggggaaatagttctcctggtaattttgtcatttttcagggaggatagcaacttttaattatcattcctacccttttcactatcatatcatttcttgtccaactatgtgatgaagatcagcagatacaatatacaagtattgtttactctccctagcgtgcatggcattccctcttcagggatatagcttccttgccagtagacaaaccagagctgcaagagctacataaattaaacaaagaccattaatactaccacgacatttggatggtctttattagccattcaaactccaacttcagtaaatttcattgtttttataactccattcaacataacaattaccactagtacttattagtgtctcagtataggcaatggcttctatgaacaaatctctcagtatagactgtgctcattc

SEQ ID NO: 478 - DNA, 259 bp, Solanum tuberosum
  source 1..259: mol_type = unassigned DNA
gggaggatagcaacatttaattatcattcctaccctcttcactatcatgtcatttcttgtccaactatgtgatgaagatcagcagataaaatatacaagtattatttactctccctagactcactacgtgcatggcattccctcttcagggatatagcttccttgccagtcgacaaaccagagctgcaagagctacataaattaaacaaagaccattaatactaccacgacatttggatggtctttattagccattcaa

SEQ ID NO: 479 - DNA, 491 bp, Solanum tuberosum
  source 1..491: mol_type = unassigned DNA
ccaccttccccccatggtgcatcctgtgtaacttcaaggaaagggaggctcatataggaaaacagaagaacagaaaagtcaacttaacggggaaatagttctcctggtaattttgtcatttttcagggaggatagcaacttttaattatcattcctacccttttcactatcatatcatttcttgtccaactatgtgatgaagatcagcagatacaatatacaagtattgtttactctccctagaccaacgtgcatggcattccctcttcagggatatagcttccttgccagtagacaaaccagagctgcaagagctacataaattaaacaaagaccattaatactaccacgacatttggatggtctttattagccattcaaactccaacttcagtaaatttcattgtttttataactccattcaacataacaattaccactagtacttattagtgtctcagtataggcaatggcttctatgaacaaatctc

SEQ ID NO: 480 - DNA, 488 bp, Solanum tuberosum
  source 1..488: mol_type = unassigned DNA
gctacacgccttcttctcgatggaatcaactgcagccgattcaaggtcatctatgtattcgacctccggacttctcactgtgtcagaagttgacatagattcatccatggagactaatgatccatctgagtgagatggagaaatgtccatgctgcaggtggcaggaagaggaatggcagtgatcttaggcacaactgcatcacttctttgaatagaaacaggtttgctgcttggtctgaggaaatgttcttcttaatgcttacaatcttggacgtacatggtgccttccaaagtcaaaaatgattccaagttagtcacaacttcagatacaagaagcaatcttttttcaatttacacacattgcaaaacagtatattaaataaaaggttaatcccaactgtgaaaactaaaaaatatcaaaagttgcttaacttcttctgctcatttgtgtaatgataatcaaattaagaacagtaatataatcattt

SEQ ID NO: 481 - DNA, 515 bp, Solanum tuberosum
  source 1..515: mol_type = unassigned DNA
gctacacgccttcttctcgatggaatcaactgcagccgattcaaggtcatctatgtattcgacctccggacttctcactgtgtcagaagttgacatagattcatccatggagactaatgatccatctgagtgagatggagaaatgtccatgctgcaggtggcaggaagaggaatggcagtgatcttaggcacaactgcatcacttctttgaatagaaacaggtttgctgcttggtctgacagaggaggatgcacagcagtccctgaggaaatgttcttcttaatgcttacaatcttggacgtacatggtgccttccaaagtcaaaaatgattccaagttagtcacaacttcagatacaagaagcaatcttttttcaatttacacacattgcaaaacagtatattaaataaaaggttaatcccaactgtgaaaactaaaaaatatcaaaagttgcttaacttcttctgctcatttgtgtaatgataatcaaattaagaacagtaatataatcattt

SEQ ID NO: 482 - DNA, 253 bp, Solanum tuberosum
  source 1..253: mol_type = unassigned DNA
tgtattcgttgaagttccttcataggattttcggtatacgttaagttttcacatttcaatttgactacgaagtatgtttaaattgatgttgtttatatctctctttgctgtgaggccactctacactcgaaactcaataggaaaaagcttagtaaactcaatattattaacatctggtgatgcgttttcttccactactacgttgaactgcctttattccttgttgattttgagtaatcatcttctgcaattt

SEQ ID NO: 483 - DNA, 235 bp, Solanum tuberosum
  source 1..235: mol_type = unassigned DNA
tctaatgtaatgtattcgttgaagttccttcataggattttcggtatacgttaagttttcacatttcaatttgactacgaagtatgtttaaattgatgttgtttatatctctctttgctgtgaaggccactctacactcgaaactcaataggaaaaagcttagtaaactcaatattattaacatctggtgatgcgttttcttccactactacgttgaactgcctttattccttgt

SEQ ID NO: 484 - DNA, 196 bp, Solanum tuberosum
  source 1..196: mol_type = unassigned DNA
ccctcttcactatcatatcatttcttgtccaactatgtgatgaagatcagcagataaaatatacaagtattatttactctccctagactcacctacgtgcttggcattccctcttcagggatatagcttccttgccagtcgacaaaccagagctgcaagagctacataaattaaacaaagaccattaatactacca

SEQ ID NO: 485 - DNA, 234 bp, Solanum tuberosum
  source 1..234: mol_type = unassigned DNA
cattcctacccccttcactatcatatcatatcttgtccaactatgtgatgaagatcagtagataaaatatacaagtattgtttactctccctagactcacctacgtgcatggcattccctcttcagggatatagcttccttgccggtcgacaaaccagagctgcaagagctacataaattaaacaaagaccattaatactaccacaacatttggatggtctttattagccattc

SEQ ID NO: 486 - DNA, 288 bp, Solanum tuberosum
  source 1..288: mol_type = unassigned DNA
tggagactaatgatccatctgagtgagatggagaaatgtccatgctgcaggtggcaggaagaggaatggcagtgatcttaggcacaactgcatcacttctttgaatagaaacaggtttgctgcttggtctgacagaggaggatgcgcagtccctgaggaaatgttcttcttaatgcttacaatcttggacgtacatggtgccttccaaagtcaaaaatgattccaagttagtcacaacttcagatacaagaagcaatcttttttcaatttacacacattgcaaaacag

SEQ ID NO: 487 - DNA, 294 bp, Solanum tuberosum
  source 1..294: mol_type = unassigned DNA
tggagactaatgatccatctgagtgagatggagaaatgtccatgctgcaggtggcaggaagaggaatggcagtgatcttaggcacaactgcatcacttctttgaatagaaacaggtttgctgcttggtctgacagaggaggatgctgaacagcagtccctgaggaaatgttcttcttaatgcttacaatcttggacgtacatggtgccttccaaagtcaaaaatgattccaagttagtcacaacttcagatacaagaagcaatcttttttcaatttacacacattgcaaaacag

SEQ ID NO: 488 - DNA, 239 bp, Solanum tuberosum
  source 1..239: mol_type = unassigned DNA
taatgttctgtattcgttgaagttccttcataggattttttgtatacgttaagttttcacatttcaatttggctacgaagaatgtttaaattgatgttgtttatatctctctctttgctgtgaagcggccactctacactcgaaactcaataggaaaaagcttagtaaactcaatataattaacatctggtgatgcgttttcttccactactacgttgaactgcctttattccttgttg

SEQ ID NO: 489 - DNA, 415 bp, Solanum tuberosum
  source 1..415: mol_type = unassigned DNA
gttttgcgttttaaactttcatatatcgcaattcctgtagtggatcatcttttggtcttttgcttttctgtatctaatgtaatgtattcgttgaagttccttcataggattttcggtatacgttaagttttcacatttcaatttgactacgaagaatgtttaaattgatgctgtttatatctctctctttgctgtgaacactctacactcgaaactcaataggaaaaagcttagtaaactcaatattattaacatctggtgaggcgttgtcttccactactacgttgaactgcctttattccttgttgattttgagtaatcatcttctgcaatttcttttgtcacagtgaacaaatcttaaatccatcagtcccgatggctctgaggctctctggaattctcatgggtatgcttc

SEQ ID NO: 490 - DNA, 278 bp, Solanum tuberosum
  source 1..278: mol_type = unassigned DNA
taattttgtcacttttcagggaggatagcaacatttaattatcattcctaccctcttcactatcatgtcatttcttgtcccactctgtgatgaagatcagcagataaaatatacaagtattatttactctccctagactcacctacgtgcatggcattccctcttcagggatatagcttccttgccagtcgacaaaccagagctgcaagagctacataaattaaacaaagaccattaatactaccacgacatttggatggtctttattagccattcaa

SEQ ID NO: 491 - DNA, 282 bp, Solanum tuberosum
  source 1..282: mol_type = unassigned DNA
tttttcagggaggatagcaacttttaattatcattcctacccttttcactatcatatcatttcttgtccaactatgtgatgaagatcagcagatacaatatacaagtattgtttactctccctagactcacctacgtgcatggcattccctcttcagggatatagcttccttgccagtagacaaaccagagctgcaagagctacataaattaaacaaagaccattaatactaccacgacatttggatggtctttattagccattcaaactccaacttcagta

SEQ ID NO: 492 - skipped

SEQ ID NO: 493 - DNA, 240 bp, Solanum tuberosum
  source 1..240: mol_type = unassigned DNA
ttttgctttctgtatctaatgtaatgtattcgttgaagttccttcataggattttcggtatacgttaagttttcacatttcaatttgactacgaagtatgtttaaattgatgttgtttatatctcggccactctacactcgaaactcaataggaaaaagcttagtaaactcaatattattaacatctggtgatgcgttttcttccactactacgttgaactgcctttattccttgttgat

SEQ ID NO: 494 - DNA, 220 bp, Solanum tuberosum
  source 1..220: mol_type = unassigned DNA
atgtaatgtattcgttgaagttccttcataggattttcggtatacgttaagttttcacatttcaatttgactacgaagtatgtttaaattgatgttgtttatatctctctttgctgggaaaaagcttagtaaactcaatattattaacatctggtgatgcgttttcttccactactacgttgaactgcctttattccttgttgattttgagtaatcatct

SEQ ID NO: 495 - DNA, 240 bp, Solanum tuberosum
  source 1..240: mol_type = unassigned DNA
ttatcattcctacccccttcactatcatatcatatcttgtccaactatgtgatgaagatcagtagataaaatatacaagtattgtttactctccctagactcacctacgtgcatggcattccctcttcagggatatagcttccttgccggtcgacaaaccagagctgcaagagctacataaattaaacaaagaccattaatactaccacaacatttggatggtctttattagccattcaa

SEQ ID NO: 496 - DNA, 208 bp, Solanum tuberosum
  source 1..208: mol_type = unassigned DNA
attcctaccctcttcactatcatatcatttcttgtccaactatgtgatgaagatcagcagataaaatatacaagtattatttactctccctagactctacgtgcttggcattccctcttcagggatatagcttccttgccagtcgacaaaccagagctgcaagagctacataaattaaacaaagaccattaatactaccacgacattt